(19) United States
(12) Patent Application Publication
(10) Pub. No.: US 2008/0194617 A1
(43) Pub. Date: Aug. 14, 2008

(54) Fused ring compound

(76) Inventors: Taisuke Tawaraishi, Osaka (JP); Hiroshi Imoto, Osaka (JP); Nobuo Cho, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 12/068,442

(22) Filed: Feb. 6, 2008

(30) Foreign Application Priority Data
Feb. 9, 2007 (JP) 2007-031221

(51) Int. Cl. A61K 31/437; C07D 403/02; A61K 31/4155; A61P 3/08; C07D 471/04

(52) U.S. Cl. 514/300; 548/364.7; 514/406; 546/113

(57) ABSTRACT

The present invention provides an agent for the prophylaxis or treatment of diabetes, which has a superior hypoglycemic action, and is associated with a fewer side effects such as body weight gain and the like.

The present invention relates an agent for the prophylaxis or treatment of diabetes, which comprises a compound represented by

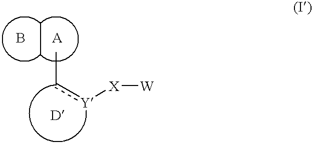

wherein each symbol is as defined in the description, or a salt thereof or a prodrug thereof.

### TECHNICAL FIELD

The present invention relates to a fused ring compound as an agent for the prophylaxis or treatment of diabetes.

### BACKGROUND OF THE INVENTION

As a fused ring compound, the compounds described in the following literatures are known.

(1) As a compound having endothelin-converting enzyme inhibitory action, WO2006/075955 discloses a compound represented by the formula:

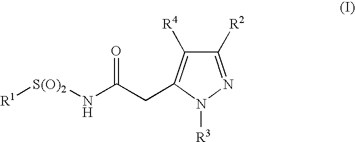

wherein
R¹ is an optionally substituted aryl group or an optionally substituted heteroaryl group;
R² and R⁴ are independently a hydrogen atom, a halogen atom, a C₁₋₆ alkyl group and the like; and
R³ is an optionally substituted aryl group, an optionally substituted heteroaryl group or a C₃₋₇ cycloalkyl group.
(2) As a compound having 15-lipoxygenase inhibitory action, US2005/0070589 discloses a compound represented by the formula:

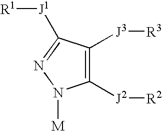

wherein
J¹ is a bond, —C(O)—, —OC(O)—, —C(O)O—, —NR⁴—, —NR⁴—CO— or —CONR⁴—;
J² is a bond, —CO—, —OC(O)—, —C(O)O—, —NR^{4a}—, —NR^{4a}—C(O)— or —C(O)NR^{4a}—;
J³ is an alkylene group, an alkenylene group, an alkynylene group and the like, each of which is optionally substituted by an alkyl group and the like;
R¹ and R² are independently a hydrogen atom, a cycloalkyl group, a heterocyclic group, an aryl group, a heteroaryl group and the like, each of which is optionally substituted by an alkyl group and the like;
R³ is —NR^{3a}SO₂Z, —NR^{3a}C(O)OZ, —NR^{3a}C(O)Z, —NR^{3a}C(O)NR^{3b}Z and the like;
R^{3a}, R^{3b}, R⁴ and R^{4a} are independently a hydrogen atom, an alkyl group and the like;
Z is —NR⁵R⁶, —C(O)R⁷, —C(O)OR⁷ and the like;
R⁵ and R⁶ are independently a hydrogen atom, an alkyl group and the like;
R⁷ is a hydrogen atom, an alkyl group and the like; and
M is a hydrogen atom, an alkyl group and the like,
provided that a compound wherein R¹-J¹- and R²-J²- are both hydrogen atoms are excluded.
(3) As a compound having 15-lipoxygenase inhibitory action, US2005/0070588 discloses a compound represented by the formula:

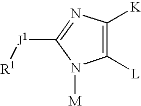

wherein
one of K and L is -J²-R² and the other is -J³-R³;
J¹ is a bond, —C(O)—, —OC(O)—, —C(O)O—, —NR⁴—, —NR⁴—CO— or —CONR⁴—;
J² is a bond, —CO—, —OC(O)—, —C(O)O—, —NR^{4a}—, —NR^{4a}—C(O)— or —C(O)NR^{4a}—;
J³ is an alkylene group, an alkenylene group, an alkynylene group and the like, each of which is optionally substituted by an alkyl group and the like;
R¹ and R² are independently a hydrogen atom, a cycloalkyl group, a heterocyclic group, an aryl group, a heteroaryl group and the like, each of which is optionally substituted by an alkyl group and the like;
R³ is —NR^{3a}SO₂Z, —NR^{3a}C(O)OZ, —NR^{3a}C(O)Z, —NR^{3a}C(O)NR^{3b}Z and the like;
R^{3a}, R^{3b}, R⁴ and R^{4a} are independently a hydrogen atom, an alkyl group and the like;
Z is —NR⁵R⁶, —C(O)R⁷, —C(O)OR⁷ and the like;
R⁵ and R⁶ are independently a hydrogen atom, an alkyl group and the like;
R⁷ is a hydrogen atom, an alkyl group and the like; and
M is a hydrogen atom, an alkyl group and the like,
provided that a compound wherein R¹-J¹- and R²-J²- are both hydrogen atoms are excluded.
(4) As a therapeutic agent for neuritis, WO99/42092 discloses a compound represented by the formula:

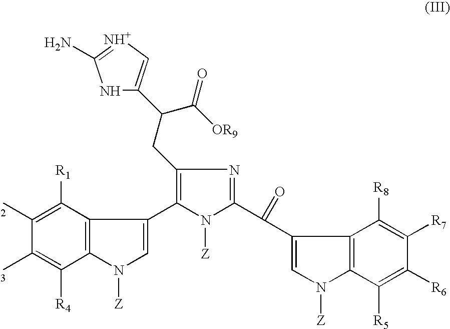

wherein
R¹ to R⁸ are independently a hydrogen atom, a hydroxy group, a halogen atom, —R, —OR, —OCOR, —OA or NZZ;
R⁹ is a C₁₋₈ alkyl group or an aryl group;
Z is a hydrogen atom, —R, a hydroxy group or —COR;
R is a C₁₋₃ alkyl group, a C₁₋₈ alkoxy group, a mesyl group or a tosyl group; and

### A is —R-phenyl.

Peroxisome proliferator-activated receptor gamma (PPARγ), which is one member of the nuclear hormone receptor superfamily represented by steroid hormone receptors and thyroid gland hormone receptors, shows an induced expression at the beginning of differentiation of adipocytes and plays an important role as a master regulator in the differentiation of adipocytes. PPARγ binds to a ligand to form a dimer with retinoid X receptor (RXR), and the dimer binds to a responsive element of a target gene in the nucleus to directly control (activate) the transcription efficiency.

(5) As a Tie2 receptor tyrosine kinase inhibitor, WO2004/013141 discloses a compound represented by the formula:

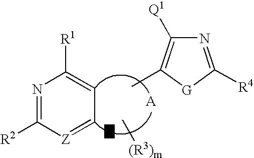

wherein
A is a 5-membered aromatic heterocycle;

### G is O, S or NR⁵;

### Z is N or CR⁶;

Q¹ is an aryl group or a heteroaryl group, each of which is optionally substituted;
R² is H, an amino and the like;
R³, R⁴, R⁵ and R⁶ are independently H, OH, a halogen atom, Q⁴-X⁵— and the like;
Q⁴ is an aryl group, an aryl-C₁₋₆ alkyl group, a heteroaryl group, a heteroaryl-C₁₋₆ alkyl group, a heterocyclyl group or a heterocyclyl-C₁₋₆ alkyl group; and
m is 0, 1 or 2.
(6) As an ERK/MAP inhibitor, WO2002/072576 discloses a compound represented by the formula:

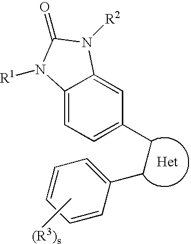

wherein

### Het is any of

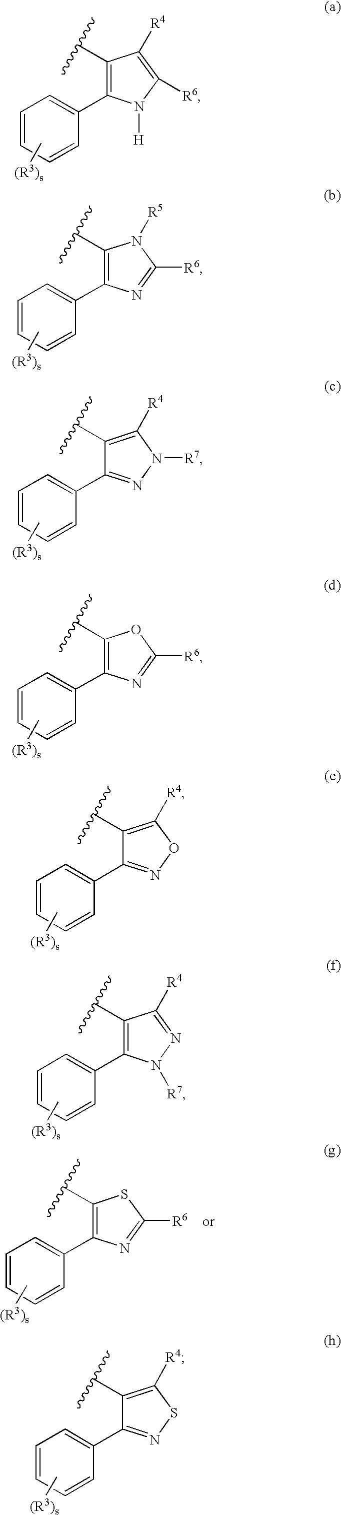

R¹ and R² are independently H, a C₁₋₆ alkyl and the like;
R³ is a halogen atom, a C₁₋₆ alkyl and the like;
R⁴ and R⁶ are independently H, a halogen atom or —(CH₂)ₙ—B—R⁹;
B is a bond, —O—, —S—, —CO— and the like;
R⁵ and R⁷ are independently H, an optionally substituted phenyl, an optionally substituted C₁₋₁₀ heteroaryl, an optionally substituted C₁₋₁₀ heterocyclyl and the like;
R⁹ is H, an optionally substituted phenyl, an optionally substituted C₁₋₁₀ heteroaryl, an optionally substituted C₁₋₁₀ heterocyclyl and the like; and
s is an integer of 0 to 5.
(7) As a catalyst, Journal of Organic Chemistry, 200.6, 71(24), pp. 9244-9247 discloses the following compound:

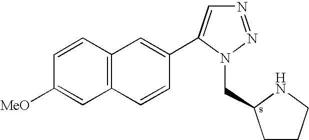

### DISCLOSURE OF THE INVENTION

There is a demand on the development of an agent for the prophylaxis or treatment of diabetes, which has a superior hypoglycemic action, and is associated with a fewer side effects such as body weight gain and the like.

The present inventors have found that a compound represented by the following formulas (I′) and a compound represented by the following formulas (I) have a superior hypoglycemic action, and are useful for the prophylaxis or treatment of diabetes, which resulted in the completion of the present invention.

Accordingly, the present invention relates to
[1] a compound represented by the formula (I′):

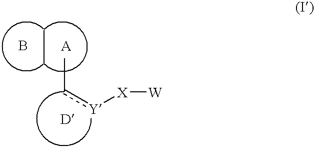

wherein
ring A and ring B are the same or different and each is an optionally substituted 5- to 7-membered monocycle;
ring D′ is an optionally substituted 5-membered monocyclic aromatic heterocycle wherein Y′ is N or C;
X is a spacer having 1 to 4 atoms in the main chain; and
W is a group represented by

### —CONR^{1a}S(O)ₘR²,

### —CONR^{1a}S(O)ₘOR²,

### —CONR^{1a}CONR^{1c}R²,

### —CONR^{1a}S(O)ₘNR^{1c}R²,

### —NR^{1b}CONR^{1a}S(O)ₘR²,

### —NR^{1b}S(O)ₘNR^{1a}COₙR²,

### —S(O)ₘNR^{1a}COₙR²,

### —S(O)ₘNR^{1a}CONR^{1c}R²,

### —OCONR^{1a}S(O)ₘR²,

### —OCONR^{1a}S(O)ₘNR^{1c}R²,

### —ONR^{1a}COₙR²,

### —OCONR^{1c}R², or

### —ONR^{1a}CONR^{1c}R²

wherein
R^{1a} and R^{1b} are the same or different and each is a hydrogen atom or a C₁₋₆ alkyl group;
R^{1c} is a hydrogen atom, a C₁₋₆ alkyl group or a C₁₋₆ alkoxy group;
R² is a hydrogen atom, an optionally substituted hydrocarbon group or an optionally substituted heterocyclic group; and
m and n are the same or different and each is an integer of 1 or 2, or
   a 5- or 6-membered heterocyclic group containing NH, which is optionally substituted,
   provided that
   1) when ring D′ is a substituted imidazole, then W should not be 2-amino-1H-imidazol-5-yl, 1H-imidazol-2-yl, 3,5-dimethyl-1H-pyrazol-4-yl and piperazin-1-yl;
   2) when ring D′ is a substituted pyrazole, and X is —CH═, then W should not be 4-oxo-2-thioxo-1,3-thiazolidin-5-ylidene, 5-oxo-2-thioxoimidazolidin-4-ylidene optionally substituted by phenyl group(s), 3-methyl-5-oxo-1,5-dihydro-4H-pyrazol-4-ylidene, 2,4,6-trioxotetrahydropyrimidin-5(2H)-ylidene and 4,6-dioxo-2-thioxotetrahydropyrimidin-5(2H)-ylidene; and
   3) 5-(6-methoxy-2-naphthyl)-1-(pyrrolidin-2-ylmethyl)-1H-1,2,3-triazole is excluded,
   or a salt thereof (hereinafter to be abbreviated as compound (1′));
[2] a compound represented by the formula (I):

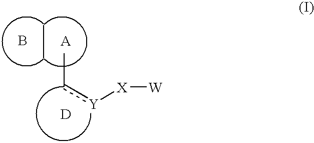

wherein
ring A and ring B are the same or different and each is an optionally substituted 5- to 7-membered monocycle;
ring D is an optionally substituted 5-membered monocycle wherein Y is N, C or CH;
X is a spacer having 1 to 4 atoms in the main chain; and
W is a group represented by

### —CONR^{1a}S(O)ₘR²,

### —CONR^{1a}CONR^{1c}R²

### —CONR^{1a}S(O)ₘNR^{1c}R²,

### —NR^{1b}CONR^{1a}S(O)ₘR²

### —S(O)ₘNR^{1a}COₙR²,

### —OCONR^{1a}S(O)ₘR²,

### —OCONR^{1a}S(O)ₘNR^{1c}R²,

### —ONR^{1a}COₙR²,

### —OCONR^{1c}R², or

### —ONR^{1a}CONR^{1c}R²

wherein
R^{1a} and R^{1b} are the same or different and each is a hydrogen atom or a C₁₋₆ alkyl group;
R^{1c} is a hydrogen atom, a C₁₋₆ alkyl group or a C₁₋₆ alkoxy group;
R² is a hydrogen atom, an optionally substituted hydrocarbon group or an optionally substituted heterocyclic group; and
m and n are the same or different and each is an integer of 1 or 2, or
   a 5- or 6-membered heterocyclic group containing NH, which is optionally substituted,
   provided that
   1) when ring D is a substituted imidazole, then W should not be an aminoimidazole; and
   2) when ring D is a substituted pyrazole, and X is —CH═, then W should not be an oxothioxothiazolidinyl and an oxothioxoimidazolidinyl,
   or a salt thereof (hereinafter to be abbreviated as compound (I));
[3] the compound of the above-mentioned [1], wherein ring D′ is an optionally substituted pyrazole;
[4] the compound of the above-mentioned [2], wherein ring D is an optionally substituted pyrazole;
[5] the compound of the above-mentioned [1] or [2], wherein X is a C₁₋₄ alkylene group or a C₂₋₄ alkenylene group;
[6] the compound of the above-mentioned [1] or [2], wherein W is a group represented by —CONR^{1a}S(O)ₘR² wherein each symbol is as defined in the above-mentioned [1];
[7] (2E)-3-[1,3-dimethyl-5-(1H-pyrrolo[2,3-b]pyridin-1-yl)-1H-pyrazol-4-yl]-N-(pentylsulfonyl)acrylamide (Example 9),
(2E)-3-[5-(5-chloro-1H-indol-1-yl)-1,3-dimethyl-1H-pyrazol-4-yl]-N-(pentylsulfonyl)acrylamide (Example 27),
(2E)-3-[1,3-dimethyl-5-(1H-pyrrolo[2,3-b]pyridin-1-yl)-1H-pyrazol-4-yl]-N-[(4-methylphenyl)sulfonyl]acrylamide (Example 33),
(2E)-3-[5-(5-chloro-1H-indol-1-yl)-1,3-dimethyl-1H-pyrazol-4-yl]-N-[(pentylamino)sulfonyl]acrylamide (Example 62), cyclopropylmethyl ({2-[5-(5-chloro-1H-indol-1-yl)-1,3-dimethyl-1H-pyrazol-4-yl]ethyl}sulfonyl)carbamate (Example 189),
butyl ({2-[5-(5-chloro-1H-pyrrolo[2,3-b]pyridin-1-yl)-1-methyl-3-(trifluoromethyl)-1H-pyrazol-4-yl]ethyl}sulfonyl)carbamate (Example 197),
(2E)-3-[1,3-dimethyl-5-(5-methyl-1H-pyrrolo[2,3-b]pyridin-1-yl)-1H-pyrazol-4-yl]-N-(pentylsulfonyl)acrylamide (Example 232),
(2E)-3-[5-(5-chloro-1H-pyrrolo[2,3-b]pyridin-1-yl)-1,3-dimethyl-1H-pyrazol-4-yl]-N-{[(cyclopropylmethyl)amino]sulfonyl}acrylamide (Example 264),
N-[(butylamino)carbonyl]-2-[5-(5-chloro-1H-pyrrolo[2,3-b]pyridin-1-yl)-3-cyclopropyl-1-methyl-1H-pyrazol-4-yl]ethanesulfonamide (Example 279),
(2E)-N-(butylsulfonyl)-3-[5-(5-chloro-1H-pyrrolo[2,3-b]pyridin-1-yl)-1,3-dimethyl-1H-pyrazol-4-yl]acrylamide (Example 283),
N-[(butylamino)carbonyl]-2-{1,3-dimethyl-5-[5-(trifluoromethyl)-1H-pyrrolo[2,3-b]pyridin-1-yl]-1H-pyrazol-4-yl}ethanesulfonamide (Example 294), or
butyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate (Example 295),
   or a salt thereof;
[8] a prodrug of compound (I′);
[9] a pharmaceutical agent comprising compound (I′) or a prodrug thereof;
[10] the pharmaceutical agent of the above-mentioned [9], which is an insulin sensitizer;
[11] the pharmaceutical agent of the above-mentioned [9], which is an agent for the prophylaxis or treatment of diabetes;
[12] a method of improving insulin resistance in a mammal, which comprises administering compound (I′) or a prodrug thereof to the mammal;
[13] a method for the prophylaxis or treatment of diabetes in a mammal, which comprises administering compound (1′) or a prodrug thereof to the mammal;
[14] use of compound (I′) or a prodrug thereof for the production of an insulin sensitizer;
[15] use of compound (I′) or a prodrug thereof for the production of an agent for the prophylaxis or treatment of diabetes;
   and the like.

### EFFECT OF THE INVENTION

According to the present invention, an agent for the prophylaxis or treatment of diabetes, which has a superior hypoglycemic action, and is associated with a fewer side effects such as body weight gain and the like, can be provided.

### DETAILED DESCRIPTION OF THE INVENTION

The present invention is explained in detail in the following.

Unless otherwise specified, the “halogen atom” in the present specification means fluorine atom, chlorine atom, bromine atom or iodine atom.

Unless otherwise specified, the “C₁₋₃ alkylenedioxy group” in the present specification means methylenedioxy, ethylenedioxy, trimethylenedioxy or the like.

Unless otherwise specified, the “C₁₋₆ alkyl group” in the present specification means methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl or the like.

Unless otherwise specified, the “C₁₋₆ alkoxy group” in the present specification means methoxy, ethoxy, propoxy, isopropoxy, butoxy, isobutoxy, sec-butoxy, tert-butoxy or the like.

Unless otherwise specified, the “C₁₋₆ alkoxy-carbonyl group” in the present specification means methoxycarbonyl, ethoxycarbonyl, propoxycarbonyl, tert-butoxycarbonyl or the like.

Unless otherwise specified, the “C₁₋₆ alkyl-carbonyl group” in the present specification means acetyl, propanoyl, butanoyl, isobutanoyl, pentanoyl, isopentanoyl, hexanoyl or the like.

The definition of each symbol in the formulas (I′) and (I) is described in detail in the following.

W is a group represented by

### —CONR^{1a}S(O)ₘR²,

### —CONR^{1a}S(O)ₘOR²,

### —CONR^{1a}CONR^{1c}R²,

### —CONR^{1a}S(O)ₘNR^{1c}R²,

### —NR^{1b}CONR^{1a}S(O)ₘR²,

### —NR^{1b}S(O)ₘNR^{1a}COₙR²,

### —S(O)ₘNR^{1a}COₙR²,

### —S(O)ₘNR^{1a}CONR^{1c}R²,

### —OCONR^{1a}S(O)ₘR²,

### —OCONR^{1a}S(O)ₘNR^{1c}R²,

### —ONR^{1a}COₙR²,

### —OCONR^{1c}R², or

### —ONR^{1a}CONR^{1c}R²

wherein
R^{1a} and R^{1b} are the same or different and each is a hydrogen atom or a C₁₋₆ alkyl group;
R^{1c} is a hydrogen atom, a C₁₋₆ alkyl group or a C₁₋₆ alkoxy group;
R² is a hydrogen atom, an optionally substituted hydrocarbon group or an optionally substituted heterocyclic group; and
m and n are the same or different and each is an integer of 1 or 2, or
   a 5- or 6-membered heterocyclic group containing NH, which is optionally substituted.

As the “hydrocarbon group” of the “optionally substituted hydrocarbon group” for R², for example, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₃ aralkyl group, a C₈₋₁₃ arylalkenyl group and the like can be mentioned.

As the C₁₋₁₀ alkyl group, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned. Of these, a C₁₋₆ alkyl group is preferable.

As the C₂₋₁₀ alkenyl group, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned. Of these, a C₂₋₆ alkenyl group is preferable.

As the C₂₋₁₀ alkynyl group, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned. Of these, a C₂₋₆ alkynyl group is preferable.

As the C₃₋₁₀ cycloalkyl group, for example, cyclopropyl, cyclobutyl, cyclopentyl, cyclohexyl, cycloheptyl, cyclooctyl and the like can be mentioned. Of these, a C₃₋₆ cycloalkyl group is preferable.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned. Of these, a C₃₋₆ cycloalkenyl group is preferable.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned. Of these, a C₄₋₆ cycloalkadienyl group is preferable.

The above-mentioned C₃₋₁₀ cycloalkyl group, C₃₋₁₀ cycloalkenyl group and C₄₋₁₀ cycloalkadienyl group are each optionally condensed with a benzene ring to form a fused cyclic group, and as the fused cyclic group, for example, indanyl, dihydronaphthyl, tetrahydronaphthyl, fluorenyl and the like can be mentioned.

In addition, the above-mentioned C₃₋₁₀ cycloalkyl group, C₃₋₁₀ cycloalkenyl group and C₄₋₁₀ cycloalkadienyl group may be each a C₇₋₁₀ cross-linked hydrocarbon group. As the C₇₋₁₀ cross-linked hydrocarbon group, bicyclo[2.2.1]heptyl(norbornyl), bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

Moreover, the above-mentioned C₃₋₁₀ cycloalkyl group, C₃₋₁₀ cycloalkenyl group and C₄₋₁₀ cycloalkadienyl group each optionally form, together with a C₃₋₁₀ cycloalkane, a C₃₋₁₀ cycloalkene or a C₄₋₁₀ cycloalkadiene, a spiro ring group. As the C₃₋₁₀ cycloalkane, C₃₋₁₀ cycloalkene and C₄₋₁₀ cycloalkadiene, rings corresponding to the above-mentioned C₃₋₁₀ cycloalkyl group, C₃₋₁₀ cycloalkenyl group and C₄₋₁₀ cycloalkadienyl group can be mentioned. As the spiro ring groups, spiro[4.5]decan-8-yl and the like can be mentioned.

As the C₆₋₁₄ aryl group, for example, phenyl, naphthyl, anthryl, phenanthryl, acenaphthylenyl, biphenylyl and the like can be mentioned. Of these, a C₆₋₁₂ aryl group is preferable.

As the C₇₋₁₃ aralkyl group, for example, benzyl, phenethyl, naphthylmethyl, biphenylylmethyl and the like can be mentioned.

As the C₈₋₁₃ arylalkenyl group, for example, styryl and the like can be mentioned.

The C₁₋₁₀ alkyl group, C₂₋₁₀ alkenyl group and C₂₋₁₀ alkynyl group exemplified as the aforementioned “hydrocarbon group” optionally has 1 to 3 substituents at substitutable positions.

As such substituents, for example,

(1) a C₃₋₁₀ cycloalkyl group (e.g., cyclopropyl, cyclohexyl);
(2) a C₆₋₁₄ aryl group (e.g., phenyl, naphthyl) optionally substituted by 1 to 3 substituents selected from
(a) a C₁₋₆ alkyl group optionally substituted by 1 to 3 halogen atoms,
(b) a hydroxy group,
(c) a C₁₋₆ alkoxy group optionally substituted by 1 to 3 halogen atoms, and
(d) a halogen atom;
   (3) an aromatic heterocyclic group (e.g., thienyl, furyl, pyridyl, pyrazolyl, imidazolyl, tetrazolyl, oxazolyl, thiazolyl, oxadiazolyl, thiadiazolyl) optionally substituted by 1 to 3 substituents selected from
(a) a C₁₋₆ alkyl group optionally substituted by 1 to 3 halogen atoms,
(b) a hydroxy group,
(c) a C₁₋₆ alkoxy group optionally substituted by 1 to 3 halogen atoms, and
(d) a halogen atom;
   (4) a non-aromatic heterocyclic group (e.g., tetrahydrofuryl, morpholinyl, thiomorpholinyl, piperidinyl, pyrrolidinyl, piperazinyl) optionally substituted by 1 to 3 substituents selected from
(a) a C₁₋₆ alkyl group optionally substituted by 1 to 3 halogen atoms,
(b) a hydroxy group,
(c) a C₁₋₆ alkoxy group optionally substituted by 1 to 3 halogen atoms, and
(d) a halogen atom;
   (5) an amino group optionally mono- or di-substituted by substituent(s) selected from
(a) a C₁₋₆ alkyl group optionally substituted by 1 to 3 substituents selected from a halogen atom and a C₃₋₁₀ cycloalkyl group (e.g., cyclopropyl),
(b) a C₁₋₆ alkyl-carbonyl group optionally substituted by 1 to 3 halogen atoms, and
(c) a C₁₋₆ alkoxy-carbonyl group optionally substituted by 1 to 3 halogen atoms;
   (6) a C₁₋₆ alkyl-carbonyl group optionally substituted by 1 to 3 halogen atoms;
   (7) a C₁₋₆ alkoxy-carbonyl group optionally substituted by 1 to 3 substituents selected from
(a) a halogen atom, and
(b) a C₁₋₆ alkoxy group;
   (8) a C₁₋₆ alkylsulfonyl group (e.g., methylsulfonyl, ethylsulfonyl, isopropylsulfonyl) optionally substituted by 1 to 3 halogen atoms;
   (9) a carbamoyl group optionally mono- or di-substituted by C₁₋₆ alkyl group(s) optionally substituted by 1 to 3 halogen atoms;
   (10) a thiocarbamoyl group optionally mono- or di-substituted by C₁₋₆ alkyl group(s) optionally substituted by 1 to 3 halogen atoms;
   (11) a sulfamoyl group optionally mono- or di-substituted by C₁₋₆ alkyl group(s) optionally substituted by 1 to 3 halogen atoms;
   (12) a carboxy group;
   (13) a hydroxy group;
   (14) a C₁₋₆ alkoxy group optionally substituted by 1 to 3 substituents selected from
(a) a halogen atom,
(b) a carboxy group,
(c) a C₁₋₆ alkoxy group,
(d) a C₁₋₆ alkyl-carbonyl group,
(e) a C₁₋₆ alkoxy-carbonyl group,
(f) an amino group optionally mono- or di-substituted by substituent(s) selected from a C₁₋₆ alkyl group and a C₁₋₆ alkoxy-carbonyl group,
(g) a C₆₋₁₄ aryl group (e.g., phenyl), and
(h) a C₃₋₁₀ cycloalkyl group (e.g., cyclopropyl);
   (15) a C₂₋₆ alkenyloxy group (e.g., ethenyloxy) optionally substituted by 1 to 3 halogen atoms;
   (16) a C₆₋₁₄ aryloxy group (e.g., phenyloxy, naphthyloxy);
   (17) a C₁₋₆ alkyl-carbonyloxy group (e.g., acetyloxy, tert-butylcarbonyloxy);
   (18) a C₆₋₁₄ aryl-carbonyl group (e.g., benzoyl) optionally substituted by 1 to 3 substituents selected from
(a) a halogen atom, and
(b) a C₁₋₆ alkyl group optionally substituted by 1 to 3 halogen atoms;
   (19) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from a C₁₋₆ alkyl group optionally substituted by 1 to 3 halogen atoms;
   (20) a mercapto group;
   (21) a C₁₋₆ alkylthio group (e.g., methylthio, ethylthio) optionally substituted by 1 to 3 halogen atoms;
   (22) a C₇₋₁₃ aralkylthio group (e.g., benzylthio);
   (23) a C₆₋₁₄ arylthio group (e.g., phenylthio, naphthylthio);
   (24) a cyano group;
   (25) a nitro group;
   (26) a halogen atom;
   (27) a C₁₋₃ alkylenedioxy group;
   (28) an aromatic heterocyclylcarbonyl group (e.g., pyrazolylcarbonyl, pyrazinylcarbonyl, isoxazolylcarbonyl, pyridylcarbonyl, thiazolylcarbonyl) optionally substituted by 1 to 3 C₁₋₆ alkyl groups optionally substituted by 1 to 3 halogen atoms;
   (29) a hydroxyimino group optionally substituted by a C₁₋₆ alkyl group (e.g., methyl) optionally substituted by 1 to 3 C₆₋₁₄ aryl groups (e.g., phenyl);
   (30) a C₁₋₆ alkylsulfonyloxy group (e.g., methylsulfonyloxy) and the like can be mentioned. When two or more substituents are used, the substituents may be the same or different.

The C₃₋₁₀ cycloalkyl group, C₃=₁₀ cycloalkenyl group, C₄₋₁₀ cycloalkadienyl group, C₆₋₁₄ aryl group, C₇₋₁₃ aralkyl group and C₈₋₁₃ arylalkenyl group exemplified as the aforementioned “hydrocarbon group” optionally have 1 to 3 substituents at substitutable positions.

As such substituent, for example,

(1) the groups exemplified as the substituents for the aforementioned C₁₋₁₀ alkyl group and the like;
(2) a C₁₋₆ alkyl group optionally substituted by 1 to 3 substituents selected from
(a) a halogen atom,
(b) a carboxy group,
(c) a hydroxy group,
(d) a C₁₋₆ alkoxy-carbonyl group,
(e) a C₁₋₆ alkoxy group optionally substituted by silyl group(s) optionally substituted by 1 to 3 C₁₋₆ alkyl groups (e.g., trimethylsilyl),
(f) an amino group optionally mono- or di-substituted by C₁₋₆ alkyl group(s), and
(g) a C₆₋₁₄ aryl group (e.g., phenyl) optionally substituted by 1 to 3 C₁₋₆ alkoxy groups;
   (3) a C₂₋₆ alkenyl group (e.g., ethenyl, 1-propenyl) optionally substituted by 1 to 3 substituents selected from
(a) a halogen atom,
(b) a carboxy group,
(c) a hydroxy group,
(d) a C₁₋₆ alkoxy-carbonyl group,
(e) a C₁₋₆ alkoxy group, and
(f) an amino group optionally mono- or di-substituted by C₁₋₆ alkyl group(s);
   (4) a C₇₋₁₃ aralkyl group (e.g., benzyl) optionally substituted by 1 to 3 substituents selected from
(a) a C₁₋₆ alkyl group optionally substituted by 1 to 3 halogen atoms,
(b) a hydroxy group,
(c) a C₁₋₆ alkoxy group, and
(d) a halogen atom;
   and the like can be mentioned. When two or more substituents are used, the substituents may be the same or different.

As the “heterocyclic group” of the “optionally substituted heterocyclic group” for R², an aromatic heterocyclic group and a non-aromatic heterocyclic group can be mentioned.

As the aromatic heterocyclic group, for example, a 5- to 7-membered monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group can be mentioned. As the fused aromatic heterocyclic group, for example, a group derived from a fused ring wherein a ring corresponding to the 5- to 7-membered monocyclic aromatic heterocyclic group and 1 or 2 rings selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring are fused, and the like can be mentioned.

As preferable examples of the aromatic heterocyclic group,

monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 4-isothiazolyl), oxazolyl (e.g., 2-oxazolyl, 4-oxazolyl, 5-oxazolyl), isoxazolyl, oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazin-1-yl, 1,2,4-triazin-3-yl, 1,3,5-triazin-1-yl) and the like;
fused aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuranyl (e.g., 2-benzofuranyl, 3-benzofuranyl), benzothiophenyl (e.g., 2-benzothiophenyl, 3-benzothiophenyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), pyrrolopyridyl (e.g., 1H-pyrrolo[2,3-b]pyridin-1-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like;
and the like can be mentioned.

As the non-aromatic heterocyclic group, for example, a 5- to 7-membered monocyclic non-aromatic heterocyclic group containing, as a ring-constituting atom besides carbon atoms, 1 to 4 heteroatoms selected from an oxygen atom, a sulfur atom (the sulfur atom is optionally oxidized) and a nitrogen atom, and a fused non-aromatic heterocyclic group can be mentioned. As the fused non-aromatic heterocyclic group, for example, a group derived from a fused ring wherein a ring corresponding to the 5- to 7-membered monocyclic non-aromatic heterocyclic group and 1 or 2 rings selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring are fused, a group wherein the above-mentioned group is partially saturated, and the like can be mentioned. In addition, as the non-aromatic heterocyclic group, a group wherein any of ring-constituting carbon atoms on the ring of the above-mentioned non-aromatic heterocyclic group is substituted by 1 to 3 oxo groups and/or thioxo groups, can be mentioned.

As preferable examples of the non-aromatic heterocyclic group,

monocyclic non-aromatic heterocyclic groups such as tetrahydrofuryl (e.g., 2-tetrahydrofuryl), dihydropyrrolyl (e.g., 2,3-dihydro-1H-pyrrol-1-yl), pyrrolidinyl (e.g., 1-pyrrolidinyl), 1,1-dioxidotetrahydrothienyl (e.g., 1,1-dioxidotetrahydro-3-thienyl), piperidinyl (e.g., piperidino), morpholinyl (e.g., morpholino), thiomorpholinyl (e.g., thiomorpholino), 1,1-dioxidothiomorpholinyl (e.g., 1,1-dioxidothiomorpholino), piperazinyl (e.g., 1-piperazinyl), hexamethyleniminyl (e.g., hexamethylenimin-1-yl), oxazolinyl (e.g., 2,5-dihydrooxazol-3-yl, 3,4-dihydrooxazol-3-yl), thiazolinyl (e.g., 2,5-dihydrothiazol-3-yl, 3,4-dihydrothiazol-3-yl), imidazolinyl (e.g., 2-imidazolin-3-yl), oxazolidinyl (e.g., oxazolidin-3-yl), thiazolidinyl (e.g., thiazolidin-3-yl), imidazolidinyl (e.g., imidazolidin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), thioxooxazolidinyl (e.g., 2-thioxo-1,3-oxazolidin-5-yl), tetrahydropyranyl (e.g., 4-tetrahydropyranyl), tetrahydrothiopyranyl (e.g., 4-tetrahydrothiopyranyl), 1,1-dioxidotetrahydrothiopyranyl (e.g., 1,1-dioxidotetrahydrothiopyran-4-yl), pyrazolinyl (e.g., pyrazolin-3-yl), pyrazolidinyl (e.g., pyrazolidin-1-yl), oxotetrahydropyridazinyl (e.g., 3-oxo-2,3,4,5-tetrahydropyridazin-4-yl) and the like;
fused non-aromatic heterocyclic groups such as dihydroisoindolyl (e.g., 1,3-dihydro-2H-isoindol-2-yl), dihydrobenzofuranyl (e.g., 2,3-dihydro-1-benzofuran-5-yl), dihydrobenzodioxinyl (e.g., 2,3-dihydro-1,4-benzodioxin-2-yl), dihydrobenzodioxepinyl (e.g., 3,4-dihydro-2H-1,5-benzodioxepin-2-yl), tetrahydrobenzofuranyl (e.g., 4,5,6,7-tetrahydro-1-benzofuran-3-yl), tetrahydrobenzothiazolyl (e.g., 4,5,6,7-tetrahydro-1-benzothiazol-2-yl), tetrahydrobenzoxazolyl (e.g., 4,5,6,7-tetrahydro-1-benzoxazol-2-yl), chromenyl (e.g., 4H-chromen-2-yl, 2H-chromen-3-yl), dihydroquinolinyl (e.g., 1,2-dihydroquinolin-2-yl), tetrahydroquinolinyl (e.g., 1,2,3,4-tetrahydroquinolin-2-yl), dihydroisoquinolinyl (e.g., 1,2-dihydroisoquinolin-2-yl), tetrahydroisoquinolinyl (e.g., 1,2,3,4-tetrahydroisoquinolin-4-yl, 1,2,3,4-tetrahydroisoquinolin-2-yl), dihydrophthalazinyl (e.g., 1,4-dihydrophthalazin-4-yl), tetrahydroindazolyl (e.g., 4,5,6,7-tetrahydro-2H-indazol-2-yl), tetrahydroquinazolinyl (e.g., 5,6,7,8-tetrahydroquinazolin-6-yl), tetrahydrothiazolopyridinyl (e.g., 4,5,6,7-tetrahydrothiazolo[5.4-c]pyridin-6-yl), tetrahydroimidazopyridinyl (e.g., 1,2,3,4-tetrahydroimidazo[4.5-c]pyridin-2-yl), tetrahydropyrazolopyridinyl (e.g., 1,2,3,4-tetrahydropyrazolo[3.4-c]pyridin-2-yl), tetrahydrotriazolopyrazinyl (e.g., 1,2,3,4-tetrahydrotriazolo[4.3-a]pyrazin-2-yl), tetrahydroimidazopyrazinyl (e.g., 1,2,3,4-tetrahydroimidazo[1.2-a]pyrazin-2-yl, 1,2,3,4-tetrahydroimidazo[3.4-a]pyrazin-2-yl), tetrahydropyridopyrimidinyl (e.g., 5,6,7,8-tetrahydropyrido[5.4-c]pyrimidin-6-yl) and the like;
can be mentioned.

The “heterocyclic group” of the “optionally substituted heterocyclic group” for R² optionally has 1 to 3 substituents at substitutable positions. As such substituents, those similar to the substituents which the C₃₋₁₀ cycloalkyl group and the like exemplified as the “hydrocarbon group” of the “optionally substituted hydrocarbon group” for R² optionally has, can be mentioned. When two or more substituents are used, the substituents may be the same or different.

R^{1a} is preferably a hydrogen atom.

R^{1b} is preferably a hydrogen atom.

R^{1c} is preferably a hydrogen atom or a C₁₋₆ alkyl group (preferably methyl), more preferably a hydrogen atom.

R² is preferably

(1) a hydrogen atom,
(2) a C₁₋₁₀ alkyl group (preferably methyl, ethyl, propyl, butyl, isobutyl, pentyl, isopentyl, neopentyl, 1-ethylpropyl, 1-propylbutyl, 4-methylpentyl) optionally substituted by 1 to 3 substituents selected from
(a) a C₆₋₁₄ aryl group (preferably phenyl) optionally substituted by 1 to 3 C₁₋₆ alkoxy groups (preferably methoxy),
(b) a C₁₋₆ alkoxy group (preferably isopropoxy),
(c) a C₁₋₆ alkoxy-carbonyl group (preferably ethoxycarbonyl),
(d) a C₃₋₁₀ cycloalkyl group (preferably cyclopropyl, cyclohexyl),
(e) a hydroxy group, and
(f) a halogen atom (preferably fluorine atom);
   (3) a C₆₋₁₄ aryl group (preferably phenyl) optionally substituted by 1 to 3 substituents selected from
(a) a halogen atom (preferably chlorine atom),
(b) a C₁₋₆ alkyl group (preferably methyl, butyl) optionally substituted by 1 to 3 halogen atoms (preferably fluorine atom),
(c) a C₁₋₆ alkoxy group (preferably methoxy), and
(d) a hydroxy group;
   (4) a C₃₋₁₀ cycloalkyl group (preferably cyclopropyl, cyclohexyl);
   (5) an aromatic heterocyclic group (preferably furyl, thienyl, imidazolyl) optionally substituted by 1 to 3 C₁₋₆ alkyl groups (preferably methyl); or
   (6) a non-aromatic heterocyclic group (preferably dihydrobenzofuranyl, morpholinyl, piperidinyl) optionally substituted by 1 to 3 substituents selected from
(a) an oxo group,
(b) a hydroxy group,
(c) a C₁₋₆ alkyl group (preferably methyl), and
(d) a C₁₋₃ alkylenedioxy group (preferably ethylenedioxy).

m is preferably 2.

The “5- or 6-membered heterocyclic group containing NH” of the “5- or 6-membered heterocyclic group containing NH, which is optionally substituted” for W is a 5- or 6-membered heterocyclic group containing, as a ring-constituting member, at least one non-substituted NH (i.e., —NH—), and further containing, as a ring-constituting atom, 4 or 5 atoms selected from a carbon atom (the carbon atom is optionally substituted by an oxo group or a thioxo group), an oxygen atom, a sulfur atom (the sulfur atom is optionally oxidized) and a nitrogen atom. For example, a 5- or 6-membered aromatic heterocyclic group and a 5- or 6-membered non-aromatic heterocyclic group, each of which contains NH, can be mentioned.

As specific preferable examples of the “5- or 6-membered aromatic heterocyclic group containing NH”, pyrrolyl, imidazolyl, pyrazolyl, triazolyl, tetrazolyl and the like can be mentioned.

As specific preferable examples of the “5- or 6-membered non-aromatic heterocyclic group containing NH”, pyrrolinyl, 2,5-dioxopyrrolinyl, pyrrolidinyl, 2-oxopyrrolidinyl, 2,5-dioxopyrrolidinyl, pyrazolinyl, pyrazolidinyl, imidazolinyl, imidazolidinyl, 2-oxoimidazolidinyl, 2,4-dioxoimidazolidinyl, triazolinyl, triazolidinyl, tetrazolinyl, tetrazolidinyl, piperidinyl, 2,6-dioxopiperidinyl, morpholinyl, thiomorpholinyl, piperazinyl, 2-oxopiperazinyl, hexamethyleniminyl, oxazolinyl, oxooxazolinyl, oxazolidinyl, 2,4-dioxooxazolidinyl, thiazolinyl, thiazolidinyl, 2,4-dioxothiazolidinyl, isoxazolinyl, isoxazolidinyl, isothiazolinyl, isothiazolidinyl, 1,1-dioxidoisothiazolidinyl, 1,1-dioxido-3-oxoisothiazolidinyl, oxadiazolinyl, oxadiazolidinyl, oxooxadiazolinyl, oxooxadiazolidinyl, thiadiazolinyl, thiadiazolidinyl, 1,1-dioxido-3-oxothiadiazolidinyl, dihydropyridyl, tetrahydropyridyl, dihydropyrimidinyl, tetrahydropyrimidinyl, 2,6-dioxohexahydropyrimidinyl, dihydropyridazinyl, tetrahydropyridazinyl, dihydropyrazinyl, tetrahydropyrazinyl, 1,1-dioxido-1,2-thiazinanyl, 1,1-dioxido-3-oxo-1,2-thiazinanyl and the like can be mentioned.

The “5- or 6-membered heterocyclic group containing NH” of the “5- or 6-membered heterocyclic group containing NH, which is optionally substituted” for W optionally has 1 to 3 substituents at substitutable positions. As such substituents, those similar to the substituents which the C₃₋₁₀ cycloalkyl group and the like exemplified as the “hydrocarbon group” of the “optionally substituted hydrocarbon group” for R² optionally has, can be mentioned. When two or more substituents are used, the substituents may be the same or different.

As preferable substituents for “5- or 6-membered heterocyclic group containing NH”, a C₁₋₆ alkyl group (preferably propyl, isopropyl) and the like can be mentioned.

The “5- or 6-membered heterocyclic group containing NH” of the “5- or 6-membered heterocyclic group containing NH, which is optionally substituted” for W is preferably a 5- or 6-membered non-aromatic heterocyclic group containing NH, more preferably oxooxadiazolinyl (preferably 5(4H)-oxo-1,2,4-oxadiazol-3-yl), 2,4-dioxothiazolidinyl (preferably 2,4-dioxothiazolidin-5-yl), 2,4-dioxoimidazolidinyl (preferably 2,4-dioxoimidazolidin-3-yl), 2-oxopiperazinyl (preferably 2-oxopiperazin-1-yl) or 1,1-dioxido-3-oxothiadiazolidinyl (preferably 1,1-dioxido-3-oxo-1,2,5-thiadiazolidin-5-yl).

W is preferably a group represented by

### —CONR^{1a}S(O)ₘR²,

### —CONR^{1a}S(O)ₘOR²,

### —CONR^{1a}S(O)ₘNR^{1c}R²,

### —NR^{1b}CONR^{1a}S(O)ₘR²,

### —NR^{1b}S(O)ₘNR^{1a}COₙR²,

### —S(O)ₘNR^{1a}COₙR²,

### —S(O)ₘNR^{1a}CONR^{1c}R²,

### —OCONR^{1a}S(O)ₘR²,

### —OCONR^{1a}S(O)ₘNR^{1c}R²,

### —ONR^{1a}COₙR²,

### —OCONR^{1c}R², or

### —ONR^{1a}CONR^{1c}R²

wherein each symbol is as defined above, or
   a 5- or 6-membered non-aromatic heterocyclic group containing NH, which is optionally substituted. Of these, it is preferably a group represented by

### —CONR^{1a}S(O)ₘR²,

### —CONR^{1a}S(O)ₘNR^{1c}R²,

### —S(O)ₘNR^{1a}COₙR², or

### —S(O)ₘNR^{1a}CONR^{1c}R²

wherein each symbol is as defined above,
   particularly preferably a group represented by —CONR^{1a}S(O)ₘR² wherein each symbol is as defined above.

As specific preferable examples of W,

(A) a group represented by

### —CONR^{1a}S(O)ₘR²,

### —CONR^{1a}S(O)ₘOR²,

### —CONR^{1a}S(O)ₘNR^{1c}R²,

### —NR^{1b}CONR^{1a}S(O)ₘR²,

### —NR^{1b}S(O)ₘNR^{1a}COₙR²,

### —S(O)ₘNR^{1a}COₙR²,

### —S(O)ₘNR^{1a}CONR^{1c}R²,

### —OCONR^{1a}S(O)ₘR²,

### —OCONR^{1a}S(O)ₘNR^{1c}R²,

### —ONR^{1a}COₙR²,

### —OCONR^{1c}R², or

### —ONR^{1a}CONR^{1c}R²

wherein
R^{1a} is a hydrogen atom;
R^{1b} is a hydrogen atom;
R^{1c} is a hydrogen atom, a C₁₋₆ alkyl group (preferably methyl) or a C₁₋₆ alkoxy group (preferably propoxy);
R² is
(1) a hydrogen atom,
(2) a C₁₋₁₀ alkyl group (preferably methyl, ethyl, propyl, butyl, isobutyl, pentyl, isopentyl, neopentyl, 1-ethylpropyl, 1-propylbutyl, 4-methylpentyl) optionally substituted by 1 to 3 substituents selected from
   (a) a C₆₋₁₄ aryl group (preferably phenyl) optionally substituted by 1 to 3 C₁₋₆ alkoxy groups (preferably methoxy),
   (b) a C₁₋₆ alkoxy group (preferably isopropoxy),
   (c) a C₁₋₆ alkoxy-carbonyl group (preferably ethoxycarbonyl),
   (d) a C₃₋₁₀ cycloalkyl group (preferably cyclopropyl, cyclohexyl),
   (e) a hydroxy group, and
   (f) a halogen atom (preferably fluorine atom);
(3) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(4) a C₃₋₁₀ cycloalkyl group (preferably cyclopropyl, cyclohexyl);
(5) an aromatic heterocyclic group (preferably furyl, thienyl, imidazolyl) optionally substituted by 1 to 3 C₁₋₆ alkyl groups (preferably methyl); or
(6) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy);
m is 2; and
n is 1 or 2, and
   (B) a 5- or 6-membered non-aromatic heterocyclic group containing NH [preferably oxooxadiazolinyl (preferably 5(4H)-oxo-1,2,4-oxadiazol-3-yl), 2,4-dioxothiazolidinyl (preferably 2,4-dioxothiazolidin-5-yl), 2,4-dioxoimidazolidinyl (preferably 2,4-dioxoimidazolidin-3-yl), 2-oxopiperazinyl (preferably 2-oxopiperazin-1-yl), 1,1-dioxido-3-oxothiadiazolidinyl (preferably 1,1-dioxido-3-oxo-1,2,5-thiadiazolidin-5-yl)] optionally substituted by 1 to 3 C₁₋₆ alkyl groups (preferably propyl, isopropyl),
   can be mentioned.

As specific more preferable examples of W, a group represented by

### —CONR^{1a}S(O)ₘR²,

### —CONR^{1a}S(O)ₘNR^{1c}R²,

### —S(O)ₘNR^{1a}COₙR², or

### —S(O)ₘNR^{1a}CONR^{1c}R²

wherein
R^{1a} is a hydrogen atom;
R^{1c} is a hydrogen atom, a C₁₋₆ alkyl group (preferably methyl) or a C₁₋₆ alkoxy group (preferably propoxy);
R² is
(1) a hydrogen atom,
(2) a C₁₋₁₀ alkyl group (preferably methyl, ethyl, propyl, butyl, isobutyl, pentyl, isopentyl, neopentyl, 1-propylbutyl, 4-methylpentyl) optionally substituted by 1 to 3 substituents selected from
   (a) a C₆₋₁₄ aryl group (preferably phenyl),
   (b) a C₁₋₆ alkoxy group (preferably isopropoxy),
   (c) a C₁₋₆ alkoxy-carbonyl group (preferably ethoxycarbonyl),
   (d) a C₃₋₁₀ cycloalkyl group (preferably cyclopropyl, cyclohexyl),
   (e) a hydroxy group, and
   (f) a halogen atom (preferably fluorine atom);
(3) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(4) a C₃₋₁₀ cycloalkyl group (preferably cyclopropyl, cyclohexyl);
(5) an aromatic heterocyclic group (preferably furyl, thienyl, imidazolyl) optionally substituted by 1 to 3 C₁₋₆ alkyl groups (preferably methyl); or
(6) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy);
m is 2; and
n is 1 or 2,
   can be mentioned.

As specific particularly preferable examples of W, a group represented by

### —CONR^{1a}S(O)ₘR²

wherein
R^{1a} is a hydrogen atom;
R² is
(1) a C₁₋₁₀ alkyl group (preferably methyl, propyl, butyl, pentyl, 4-methylpentyl) optionally substituted by 1 to 3 substituents selected from
   (a) a C₆₋₁₄ aryl group (preferably phenyl), and
   (b) a C₃₋₁₀ cycloalkyl group (preferably cyclopropyl);
(2) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(3) a C₃₋₁₀ cycloalkyl group (preferably cyclopropyl);
(4) an aromatic heterocyclic group (preferably furyl, thienyl, imidazolyl) optionally substituted by 1 to 3 C₁₋₆ alkyl groups (preferably methyl); or
(5) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy); and
m is 2,
   can be mentioned.

Ring A and ring B are the same or different and each is an optionally substituted 5- to 7-membered monocycle. As the “5- to 7-membered monocycle” of the “optionally substituted 5- to 7-membered monocycle” for ring A or ring B, a “5- to 7-membered monocyclic aromatic ring” and a “5- to 7-membered monocyclic non-aromatic ring” can be mentioned.

As the “5- to 7-membered monocyclic aromatic ring”, benzene, a 5- to 7-membered ring (e.g., pyrrole, pyrazole, imidazole, thiophene, pyridine), from among the monocyclic aromatic heterocycle corresponding to the monocyclic aromatic heterocyclic group exemplified as the “heterocyclic group” of the “optionally substituted heterocyclic group” for R², can be mentioned.

As the “5- to 7-membered monocyclic non-aromatic ring”, a 5- to 7-membered ring (i.e., a C₅₋₇ cycloalkane, a C₅₋₇ cycloalkene and a C₅₋₇ cycloalkadiene), from among a C₃₋₁₀ cycloalkane, a C₃₋₁₀ cycloalkene and a C₄₋₁₀ cycloalkadiene corresponding to the C₃₋₁₀ cycloalkyl group, C₃₋₁₀ cycloalkenyl group and C₄₋₁₀ cycloalkadienyl group exemplified as the “hydrocarbon group” of the “optionally substituted hydrocarbon group” for R², and a 5- to 7-membered ring (e.g., pyrroline), from among a monocyclic non-aromatic heterocycle corresponding to the monocyclic non-aromatic heterocyclic group exemplified as the “heterocyclic group” of the “optionally substituted heterocyclic group” for R², can be mentioned.

In the present specification, the moiety represented by

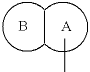

of the formula (I′) and the formula (I) is a group derived from a bicycle wherein formed by ring A and ring B having one common bond (that is, they are condensed). The bond multiplicity for ring A and that for ring B, involved in the bicycle formation, are the same. For example, when the moiety represented by

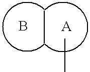

of the formula (I′) and the formula (I) is a group represented by (A)

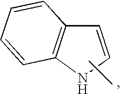

then ring A should be “pyrrole”, and ring B should be “benzene”. When the moiety is

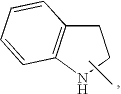

then ring A should be “pyrroline”, and ring B should be “benzene”.

As specific preferable examples of the “5- to 7-membered monocycle” of the “optionally substituted 5- to 7-membered monocycle” for ring A, benzene, a 5- to 7-membered monocyclic aromatic heterocycle (preferably pyrrole, pyrazole, imidazole, thiophene), a 5- to 7-membered monocyclic non-aromatic heterocycle (preferably pyrroline) and the like can be mentioned.

As specific preferable examples of the “5- to 7-membered monocycle” of the “optionally substituted 5- to 7-membered monocycle” for ring B, benzene, a 5- to 7-membered monocyclic aromatic heterocycle (preferably pyridine) and the like can be mentioned.

As specific preferable examples of the moiety represented by

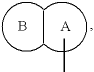

1H-indol-1-yl, 1H-indol-2-yl, 1H-indol-3-yl, 1H-indazol-1-yl, 1H-indazol-3-yl, 2H-indazol-2-yl, 1H-benzimidazol-1-yl, 1H-benzimidazol-2-yl, 1-benzothiophen-2-yl, 1-benzothiophen-3-yl, 2-benzothiophen-1-yl, 1-benzofuran-2-yl, 1-benzofuran-3-yl, 2-benzofuran-1-yl, 1H-pyrrolo[2,3-b]pyridin-1-yl, 1H-pyrrolo[2,3-b]pyridin-2-yl, 1H-pyrrolo[2,3-b]pyridin-3-yl, 2,3-dihydro-1H-pyrrolo[2,3-b]pyridin-1-yl, 2,3-dihydro-1H-pyrrolo[2,3-b]pyridin-2-yl, 2,3-dihydro-1H-pyrrolo[2,3-b]pyridin-3-yl, 1H-pyrrolo[3,2-c]pyridin-1-yl, 1H-pyrrolo[3,2-c]pyridin-2-yl, 1H-pyrrolo[3,2-c]pyridin-3-yl, 1H-pyrrolo[2,3-c]pyridin-1-yl, 1H-pyrrolo[2,3-c]pyridin-2-yl, 1H-pyrrolo[2,3-c]pyridin-3-yl, 1-naphthyl, 2-naphthyl, quinolin-5-yl, quinolin-6-yl, quinolin-7-yl, quinolin-8-yl, isoquinolin-5-yl, isoquinolin-6-yl, isoquinolin-7-yl, isoquinolin-8-yl and the like can be mentioned.

The “5- to 7-membered monocycle” of the “optionally substituted 5- to 7-membered monocycle” for ring A or ring B optionally has 1 to 3 substituents at substitutable positions. As such substituents, those similar to the substituents which the C₃₋₁₀ cycloalkyl group and the like exemplified as the “hydrocarbon group” of the “optionally substituted hydrocarbon group” for R² optionally has, can be mentioned. When two or more substituents are used, the substituents may be the same or different.

As preferable substituents for ring A and ring B,

(1) a halogen atom (preferably chlorine atom, fluorine atom, bromine atom),
(2) a hydroxy group,
(3) a cyano group,
(4) a C₁₋₆ alkyl group (preferably methyl) optionally substituted by 1 to 3 halogen atoms (preferably fluorine atom),
(5) a C₁₋₆ alkoxy group (preferably methoxy, ethoxy, isopropoxy) optionally substituted by 1 to 3 substituents selected from
(a) a C₆₋₁₄ aryl group (preferably phenyl),
(b) a C₁₋₆ alkoxy group (preferably methoxy),
(c) a C₃₋₁₀ cycloalkyl group (preferably cyclopropyl), and
(d) a C₁₋₆ alkyl-carbonyl group (preferably acetyl),
   (6) a C₃₋₁₀ cycloalkyl group (preferably cyclopropyl),
   (7) a C₁₋₆ alkylsulfonyloxy group (preferably methylsulfonyloxy),
   (8) a C₆₋₁₄ aryl group (preferably phenyl),
   (9) an aromatic heterocyclic group (preferably furyl, thienyl),
   (10) a non-aromatic heterocyclic group (preferably pyrrolidinyl),
   (11) an amino group optionally mono- or di-substituted by C₁₋₆ alkyl group(s) (preferably methyl, ethyl) optionally substituted by 1 to 3 C₃₋₁₀ cycloalkyl groups (preferably cyclopropyl)
   and the like can be mentioned.

Ring A is preferably optionally substituted benzene, an optionally substituted 5- to 7-membered monocyclic aromatic heterocycle (preferably pyrrole, pyrazole, imidazole, thiophene) or an optionally substituted 5- to 7-membered monocyclic non-aromatic heterocycle (preferably pyrroline).

As specific preferable examples of ring A, benzene, a 5- to 7-membered monocyclic aromatic heterocycle (preferably pyrrole, pyrazole, imidazole, thiophene) and a 5- to 7-membered monocyclic non-aromatic heterocycle (preferably pyrroline), each of which is optionally substituted by 1 to 3 substituents selected from a halogen atom (preferably chlorine atom) and a C₁₋₆ alkyl group (preferably methyl), can be mentioned.

Ring B is preferably an optionally substituted benzene or an optionally substituted 5- to 7-membered monocyclic aromatic heterocycle (preferably pyridine).

As specific preferable examples of ring B, benzene and a 5- to 7-membered monocyclic aromatic heterocycle (preferably pyridine), each of which is optionally substituted by 1 to 3 substituents selected from

(1) a halogen atom (preferably chlorine atom, fluorine atom, bromine atom),
(2) a hydroxy group,
(3) a cyano group,
(4) a C₁₋₆ alkyl group (preferably methyl) optionally substituted by 1 to 3 halogen atoms (preferably fluorine atom),
(5) a C₁₋₆ alkoxy group (preferably methoxy, ethoxy, isopropoxy) optionally substituted by 1 to 3 substituents selected from
(a) a C₆₋₁₄ aryl group (preferably phenyl),
(b) a C₁₋₆ alkoxy group (preferably methoxy),
(c) a C₃₋₁₀ cycloalkyl group (preferably cyclopropyl), and
(d) a C₁₋₆ alkyl-carbonyl group (preferably acetyl),
   (6) a C₃₋₁₀ cycloalkyl group (preferably cyclopropyl),
   (7) a C₁₋₆ alkylsulfonyloxy group (preferably methylsulfonyloxy),
   (8) a C₆₋₁₄ aryl group (preferably phenyl),
   (9) an aromatic heterocyclic group (preferably furyl, thienyl),
   (10) a non-aromatic heterocyclic group (preferably pyrrolidinyl), and
   (11) an amino group optionally mono- or di-substituted by C₁₋₆ alkyl group(s) (preferably methyl, ethyl) optionally substituted by 1 to 3 C₃₋₁₀ cycloalkyl groups (preferably cyclopropyl)
   can be mentioned.

Ring D is an optionally substituted 5-membered monocycle wherein Y is N, C or CH, which is a ring D-constituting atom in the formula (I). As the “5-membered monocycle” of the “optionally substituted 5-membered monocycle” for ring D, a “5-membered monocyclic aromatic ring” and a “5-membered monocyclic non-aromatic ring” can be mentioned.

As the “5-membered monocyclic aromatic ring”, a 5-membered ring (e.g., pyrazole), from among a monocyclic aromatic heterocycle corresponding to the monocyclic aromatic heterocyclic group exemplified as the “heterocyclic group” of the “optionally substituted heterocyclic group” for R², can be mentioned.

As the “5-membered monocyclic non-aromatic ring”, cyclopentane, cyclopentene, cyclopentadiene, and a 5-membered ring (e.g., pyrazolidine, pyrazoline, imidazoline, imidazolidine), from among a monocyclic non-aromatic heterocycle corresponding to the monocyclic non-aromatic heterocyclic group exemplified as the “heterocyclic group” of the “optionally substituted heterocyclic group” for R², can be mentioned.

In ring D, Y (a ring D-constituting atom) and the carbon atom on the ring D (bonded to ring A) are adjacent each other via a single bond or a double bond.

The “5-membered monocycle” of the “optionally substituted 5-membered monocycle” for ring D is preferably a 5-membered monocyclic aromatic heterocycle (preferably pyrazole) and the like.

The “5-membered monocycle” of the “optionally substituted 5-membered monocycle” for ring D optionally has 1 to 3 substituents at substitutable positions. As such substituents, those similar to the substituents which the C₃₋₁₀ cycloalkyl group and the like exemplified as the “hydrocarbon group” of the “optionally substituted hydrocarbon group” for R² optionally has, can be mentioned. When two or more substituents are used, the substituents may be the same or different.

As preferable substituents for ring D,

(1) a C₁₋₆ alkyl group (preferably methyl) optionally substituted by 1 to 3 substituents selected from a halogen atom (preferably fluorine atom) and a C₁₋₆ alkoxy group (preferably methoxy)
and the like can be mentioned.

Ring D is preferably an optionally substituted 5-membered monocyclic aromatic heterocycle, more preferably an optionally substituted pyrazole.

As specific preferable examples of ring D, pyrazole optionally substituted by 1 to 3 substituents selected from

(1) a C₁₋₆ alkyl group (preferably methyl) optionally substituted by 1 to 3 substituents selected from a halogen atom (preferably fluorine atom) and a C₁₋₆ alkoxy group (preferably methoxy)
can be mentioned.

Ring D′ is an optionally substituted 5-membered monocyclic aromatic heterocycle wherein Y′ is N or C, which is a ring D′-constituting atom in the formula (I′). As the “5-membered monocyclic aromatic heterocycle” of the “optionally substituted 5-membered monocyclic aromatic heterocycle” for ring D′, a 5-membered ring (e.g., pyrazole, imidazole, pyrrole, triazole, tetrazole, thiophene, furan, oxazole, thiazole, isoxazole, isothiazole, oxadiazole, thiadiazole), from among a monocyclic aromatic heterocycle corresponding to the monocyclic aromatic heterocyclic group exemplified as the “heterocyclic group” of the “optionally substituted heterocyclic group” for R², can be mentioned. Of these, it is preferably pyrazole, thiophene, imidazole or pyrrole, particularly preferable pyrazole (it is (i) bonded to ring A at the 5-position and bonded to X at the 4-position, (ii) bonded to ring A at the 3-position and bonded to X at the 4-position, or (iii) bonded to ring A at the 5-position and bonded to X at the 1-position, preferably (i) bonded to ring A at the 5-position and bonded to X at the 4-position).

In ring D′, Y′ (a ring D′-constituting atom) and the carbon atom on the ring D′ (bonded to ring A) are adjacent each other via a single bond or a double bond.

The “5-membered monocyclic aromatic heterocycle” of the “optionally substituted 5-membered monocyclic aromatic heterocycle” for ring D′ has 1 to 3 substituents at substitutable positions. As such substituents, those similar to the substituents which the C₃₋₁₀ cycloalkyl group and the like exemplified as the “hydrocarbon group” of the “optionally substituted hydrocarbon group” for R² optionally has, can be mentioned. When two or more substituents are used, the substituents may be the same or different.

As preferable substituents for ring D′,

(1) a C₁₋₆ alkyl group (preferably methyl, ethyl, butyl) optionally substituted by 1 to 3 substituents selected from
(a) a halogen atom (preferably fluorine atom),
(b) a C₁₋₆ alkoxy group (preferably methoxy), and
(c) a C₆₋₁₄ aryl group (preferably phenyl) optionally substituted by 1 to 3 C₁₋₆ alkoxy groups (preferably methoxy),
   (2) a C₁₋₆ alkoxy-carbonyl group (preferably t-butoxycarbonyl),
   (3) a C₃₋₁₀ cycloalkyl group (preferably cyclopropyl)
   and the like can be mentioned.

Ring D′ is preferably pyrazole, thiophene, imidazole or pyrrole, each of which is optionally substituted, more preferably an optionally substituted pyrazole (it is (i) bonded to ring A at the 5-position and bonded to X at the 4-position, (ii) bonded to ring A at the 3-position and bonded to X at the 4-position, or (iii) bonded to ring A at the 5-position and bonded to X at the 1-position, preferably (i) bonded to ring A at the 5-position and bonded to X at the 4-position).

As specific preferable examples of ring D′, a 5-membered monocyclic aromatic heterocycle (preferably pyrazole, thiophene, imidazole, pyrrole, more preferably pyrazole (it is (i) bonded to ring A at the 5-position and bonded to X at the 4-position, (ii) bonded to ring A at the 3-position and bonded to X at the 4-position, or (iii) bonded to ring A at the 5-position and bonded to X at the 1-position, preferably (i) bonded to ring A at the 5-position and bonded to X at the 4-position)) optionally substituted by 1 to 3 substituents selected from

(1) a C₁₋₆ alkyl group (preferably methyl, ethyl, butyl) optionally substituted by 1 to 3 substituents selected from
(a) a halogen atom (preferably fluorine atom),
(b) a C₁₋₆ alkoxy group (preferably methoxy), and
(c) a C₆₋₁₄ aryl group (preferably phenyl) optionally substituted by 1 to 3 C₁₋₆ alkoxy groups (preferably methoxy),
   (2) a C₁₋₆ alkoxy-carbonyl group (preferably t-butoxycarbonyl), and
   (3) a C₃₋₁₀ cycloalkyl group (preferably cyclopropyl)
   can be mentioned.

X is a spacer having 1 to 4 atoms in the main chain.

The “main chain” of the “spacer having 1 to 4 atoms in the main chain” for X is a straight chain connecting Y′ (a ring D′-constituting atom) or Y (a ring D-constituting atom) and group W, and the atom number of the main chain is counted such that the number of atoms in the main chain will be minimum. The total atom number in the spacer is not particularly limited as long as the main chain consists of 1 to 4 atoms, and the spacer optionally has 4 or more atoms. The “main chain” consists of 1 to 4 atoms selected from a carbon atom (the carbon atom is optionally substituted by oxo group(s)) and a hetero atom (e.g., O, S, N), and may be saturated or unsaturated. In addition, when group W is a “5- or 6-membered non-aromatic heterocyclic group containing NH, which is optionally substituted”, and the non-aromatic heterocyclic group is bonded to X at the ring-constituting saturated carbon atom, the group W-side terminal of the “spacer having 1 to 4 atoms in the main chain” for X may be double bond (e.g., —CH═).

As the “spacer having 1 to 4 atoms in the main chain”, for example, a C₁₋₄ alkylene group, a C₂₋₄ alkenylene group, a C₂₋₄ alkynylene group, a C₃₋₆ cycloalkylene group, —X^{1a}-Z-X^{2a}— wherein Z is NH, O or S, X^{1a} and X^{2a} are the same or different and each is a straight chain C₁₋₃ alkylene group, and the total carbon number of X^{1a} and X^{2a} is 3 or less, —X^{3a}—CH═ wherein X^{3a} is a bond or a straight chain C₁₋₃ alkylene group, and the like can be mentioned.

As specific examples of the “spacer having 1 to 4 atoms in the main chain”,

(1) a C₁₋₄ alkylene group (e.g., —CH₂—, —(CH₂)₂—, —(CH₂)₃—, —(CH₂)₄—, —CH(CH₃)—, —CH(C₂H₅)—, —CH(C₃H₇)—, —CH(i-C₃H₇)—, —CH(CH₃)—CH₂—, —CH₂CH(CH₃)—, —CH(CH₃)(CH₂)₂—, —(CH₂)₂CH(CH₃)—, —CH₂—CH(CH₃)—CH₂—, —C(CH₃)₂—, —(CH(CH₃))₂—, —CH(CH₃)—CH(CH₃)—, —CH₂—C(CH₃)₂—);
(2) a C₂₋₄ alkenylene group (e.g., —CH═CH—, —CH═CH—CH₂—, —CH₂—CH═CH—, —C(CH₃)₂—CH═CH—, —CH₂—CH═CH—CH₂—, —CH₂—CH₂—CH═CH—, —CH═CH—CH═CH—, —C(CH₃)═CH—, —CH═C(CH₃)—, —CH═C(C₂H₅)—);
(3) a C₂₋₄ alkynylene group (e.g., —C≡C—, —CH₂—C≡C—, —CH₂—C≡C—CH₂—);
(4) a C₃₋₆ cycloalkylene group (e.g., 1,2-cyclopropylene, 1,2-cyclobutylene, 1,3-cyclobutylene, 1,2-cyclopentylene, 1,3-cyclopentylene, 1,2-cyclohexylene, 1,3-cyclohexylene, 1,4-cyclohexylene);
(5) —X^{1a}-Z-X^{2a}— wherein Z is NH, O or S, X^{1a} and X^{2a} are the same or different and each is a straight chain C₁₋₃ alkylene group, and the total carbon number of X^{1a} and X^{2a} is 3 or less (e.g., —CH₂—NH—CH₂—, —CH₂—O—CH₂—, —CH₂—S—CH₂—);
(6) —X^{3a}—CH═ wherein X^{3a} is a bond or a straight chain C₁₋₃ alkylene group (e.g., —CH═, —CH₂—CH₂—CH═, —CH₂—CH₂—CH₂—CH═);
and the like can be mentioned.

X is preferably a C₁₋₄ alkylene group, a C₂₋₄ alkenylene group, a C₃₋₆ cycloalkylene group, —X^{1a}-Z-X^{2a}— or —X^{3a—CH═ wherein each symbol is as defined above, more preferably a C}₁₋₄ alkylene group or a C₂₋₄ alkenylene group.

As specific preferable examples of X,

(1) a C₁₋₄ alkylene group (preferably —CH₂—, —CH₂CH₂—, —CH₂CH₂CH₂;
(2) a C₂₋₄ alkenylene group (preferably —CH═CH—, —CH═C(CH₃)—, —CH₂—CH═CH—);
(3) a C₃₋₆ cycloalkylene group (preferably 1,2-cyclopropylene);
(4) —X^{1a}-Z-X^{2a}— wherein each symbol is as defined above (preferably —CH₂—O—CH₂—); and
(5) —X^{3a}—CH═ wherein each symbol is as defined above (preferably —CH═, —CH₂—CH₂—CH═);
more preferably
(1) a C₁₋₄ alkylene group (preferably —CH₂—, —CH₂CH₂—, —CH₂CH₂CH₂—; and
(2) a C₂₋₄ alkenylene group (preferably —CH═CH—, —CH═C(CH₃)—, —CH₂—CH═CH—),
can be mentioned.

In compound (I),

1) when ring D is a substituted imidazole, then W should not be an aminoimidazole; and
2) when ring D is a substituted pyrazole, and X is —CH═, then W should not be an oxothioxothiazolidinyl and an oxothioxoimidazolidinyl.

In compound (1′),

1) when ring D′ is a substituted imidazole, then W should not be 2-amino-1H-imidazol-5-yl, 1H-imidazol-2-yl, 3,5-dimethyl-1H-pyrazol-4-yl and piperazin-1-yl;
2) when ring D′ is a substituted pyrazole, and X is —CH═, then W should not be 4-oxo-2-thioxo-1,3-thiazolidin-5-ylidene, 5-oxo-2-thioxoimidazolidin-4-ylidene optionally substituted by phenyl group(s), 3-methyl-5-oxo-1,5-dihydro-4H-pyrazol-4-ylidene, 2,4,6-trioxotetrahydropyrimidin-5(2H)-ylidene and 4,6-dioxo-2-thioxotetrahydropyrimidin-5(2H)-ylidene; and
3) 5-(6-methoxy-2-naphthyl)-1-(pyrrolidin-2-ylmethyl)-1H-1,2,3-triazole is excluded.

As preferable examples of compound (I), the following compounds can be mentioned.

### [Compound A]

Compound (1) wherein

ring D is an optionally substituted pyrazole;

X is a C₁₋₄ alkylene group or a C₂₋₄ alkenylene group; and

W is a group represented by

### —CONR^{1a}S(O)ₘR²

wherein each symbol is as defined above.

### [Compound B]

Compound (1) wherein

ring A is benzene, a 5- to 7-membered monocyclic aromatic heterocycle (preferably pyrrole, pyrazole, imidazole, thiophene) or a 5- to 7-membered monocyclic non-aromatic heterocycle (preferably pyrroline), each of which is optionally substituted by 1 to 3 halogen atoms (preferably chlorine atom);

ring B is benzene or a 5- to 7-membered monocyclic aromatic heterocycle (preferably pyridine), each of which is optionally substituted by 1 to 3 substituents selected from

(1) a halogen atom (preferably chlorine atom, fluorine atom),
(2) a hydroxy group,
(3) a cyano group,
(4) a C₁₋₆ alkyl group (preferably methyl) optionally substituted by 1 to 3 halogen atoms (preferably fluorine atom), and
(5) a C₁₋₆ alkoxy group (preferably methoxy) optionally substituted by 1 to 3 C₆₋₁₄ aryl groups (preferably phenyl);

ring D is pyrazole optionally substituted by 1 to 3 substituents selected from

(1) a C₁₋₆ alkyl group (preferably methyl) optionally substituted by 1 to 3 substituents selected from a halogen atom (preferably fluorine atom) and a C₁₋₆ alkoxy group (preferably methoxy);

X is

(1) a C₁₋₄ alkylene group (preferably —CH₂—, —CH₂CH₂—, —CH₂CH₂CH₂—);
(2) a C₂₋₄ alkenylene group (preferably —CH═CH—, —CH═C(CH₃)—, —CH₂—CH═CH—);
(3) —X^{1a}-Z-X^{2a}— wherein each symbol is as defined above (preferably —CH₂—O—CH₂—); or
(4) —X^{3a}—CH═ wherein each symbol is as defined above (preferably —CH═, —CH₂—CH₂—CH═); and

W is

(A) a group represented by

### —CONR^{1a}S(O)ₘR²,

### —CONR^{1a}S(O)ₘNR^{1c}R²,

### —NR^{1b}CONR^{1a}S(O)ₘR²,

### —OCONR^{1a}S(O)ₘR², or

### —OCONR^{1a}S(O)ₘNR^{1c}R²

wherein
R^{1a} is a hydrogen atom;
R^{1b} is a hydrogen atom;
R^{1c} is a hydrogen atom or a C₁₋₆ alkyl group (preferably methyl);
R² is
(1) a C₁₋₁₀ alkyl group (preferably methyl, ethyl, butyl, pentyl, 1-ethylpropyl, 1-propylbutyl, 4-methylpentyl) optionally substituted by 1 to 3 substituents selected from
   (a) a C₆₋₁₄ aryl group (preferably phenyl) optionally substituted by 1 to 3 C₁₋₆ alkoxy groups (preferably methoxy),
   (b) a C₁₋₆ alkoxy group (preferably isopropoxy),
   (c) a C₁₋₆ alkoxy-carbonyl group (preferably ethoxycarbonyl), and
   (d) a C₃₋₁₀ cycloalkyl group (preferably cyclopropyl);
(2) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom), and
   (c) a C₁₋₆ alkoxy group (preferably methoxy);
(3) a C₃₋₁₀ cycloalkyl group (preferably cyclopropyl, cyclohexyl);
(4) an aromatic heterocyclic group (preferably furyl, thienyl); or
(5) a non-aromatic heterocyclic group (preferably dihydrobenzofuranyl, morpholinyl); and
m is 2, or
   (B) oxooxadiazolinyl (preferably 5(4H)-oxo-1,2,4-oxadiazol-3-yl) or 2,4-dioxothiazolidinyl (preferably 2,4-dioxothiazolidin-5-yl).

### [Compound C]

Compound B wherein

X is

(1) a C₁₋₄ alkylene group (preferably —CH₂—, —CH₂CH₂—, —CH₂CH₂CH₂—); or
(2) a C₂₋₄ alkenylene group (preferably —CH═CH—, —CH═C(CH₃)—, —CH₂—CH═CH—); and

W is a group represented by —CONR^{1a}S(O)ₘR²
wherein
R^{1a} is a hydrogen atom;
R² is
(1) a C₁₋₁₀ alkyl group (preferably methyl, pentyl, 4-methylpentyl) optionally substituted by 1 to 3 substituents selected from
   (a) a C₆₋₁₄ aryl group (preferably phenyl), and
   (b) a C₃₋₁₀ cycloalkyl group (preferably cyclopropyl);
(2) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom), and
   (c) a C₁₋₆ alkoxy group (preferably methoxy);
(3) a C₃₋₁₀ cycloalkyl group (preferably cyclopropyl);
(4) an aromatic heterocyclic group (preferably furyl, thienyl); or
(5) a non-aromatic heterocyclic group (preferably dihydrobenzofuranyl, morpholinyl); and
m is 2.

As preferable examples of compound (I′), the following compounds can be mentioned.

### [Compound BB-1]

Compound (1′) wherein

ring A is benzene, a 5- to 7-membered monocyclic aromatic heterocycle (preferably pyrrole, pyrazole, imidazole, thiophene) or a 5- to 7-membered monocyclic non-aromatic heterocycle (preferably pyrroline), each of which is optionally substituted by 1 to 3 substituents selected from a halogen atom (preferably chlorine atom) and a C₁₋₆ alkyl group (preferably methyl);

ring B is benzene or a 5- to 7-membered monocyclic aromatic heterocycle (preferably pyridine), each of which is optionally substituted by 1 to 3 substituents selected from

(1) a halogen atom (preferably chlorine atom, fluorine atom, bromine atom),
(2) a hydroxy group,
(3) a cyano group,
(4) a C₁₋₆ alkyl group (preferably methyl) optionally substituted by 1 to 3 halogen atoms (preferably fluorine atom),
(5) a C₁₋₆ alkoxy group (preferably methoxy, ethoxy, isopropoxy) optionally substituted by 1 to 3 substituents selected from
(a) a C₆₋₁₄ aryl group (preferably phenyl),
(b) a C₁₋₆ alkoxy group (preferably methoxy),
(c) a C₃₋₁₀ cycloalkyl group (preferably cyclopropyl), and
(d) a C₁₋₆ alkyl-carbonyl group (preferably acetyl),
   (6) a C₃₋₁₀ cycloalkyl group (preferably cyclopropyl),
   (7) a C₁₋₆ alkylsulfonyloxy group (preferably methylsulfonyloxy),
   (8) a C₆₋₁₄ aryl group (preferably phenyl),
   (9) an aromatic heterocyclic group (preferably furyl, thienyl),
   (10) a non-aromatic heterocyclic group (preferably pyrrolidinyl), and
   (11) an amino group optionally mono- or di-substituted by C₁₋₆ alkyl group(s) (preferably methyl, ethyl) optionally substituted by 1 to 3 C₃₋₁₀ cycloalkyl groups (preferably cyclopropyl);

ring D′ is a 5-membered monocyclic aromatic heterocycle (preferably pyrazole, thiophene, imidazole, pyrrole, more preferably pyrazole (it is (i) bonded to ring A at the 5-position and bonded to X at the 4-position, (ii) bonded to ring A at the 3-position and bonded to X at the 4-position, or (iii) bonded to ring A at the 5-position and bonded to X at the 1-position, preferably (i) bonded to ring A at the 5-position and bonded to X at the 4-position)) optionally substituted by 1 to 3 substituents selected from

(1) a C₁₋₆ alkyl group (preferably methyl, ethyl, butyl) optionally substituted by 1 to 3 substituents selected from
(a) a halogen atom (preferably fluorine atom),
(b) a C₁₋₆ alkoxy group (preferably methoxy), and
(c) a C₆₋₁₄ aryl group (preferably phenyl) optionally substituted by 1 to 3 C₁₋₆ alkoxy groups (preferably methoxy),
   (2) a C₁₋₆ alkoxy-carbonyl group (preferably t-butoxycarbonyl), and
   (3) a C₃₋₁₀ cycloalkyl group (preferably cyclopropyl);

X is

(1) a C₁₋₄ alkylene group (preferably —CH₂—, —CH₂CH₂—, —CH₂CH₂CH₂—;
(2) a C₂₋₄ alkenylene group (preferably —CH═CH—, —CH═C(CH₃)—, —CH₂—CH═CH—);
(3) a C₃₋₆ cycloalkylene group (preferably 1,2-cyclopropylene);
(4) —X^{1a}-Z-X^{2a}— wherein each symbol is as defined above (preferably —CH₂—O—CH₂—); or
(5) —X^{3a}—CH═ wherein each symbol is as defined above (preferably —CH═, —CH₂—CH₂—CH═); and

W is

(A) a group represented by

### —CONR^{1a}S(O)ₘR²,

### —CONR^{1a}S(O)ₘOR²,

### —CONR^{1a}S(O)ₘNR^{1c}R²,

### —NR^{1b}CONR^{1a}S(O)ₘR²,

### —NR^{1b}S(O)ₘNR^{1a}COₙR²,

### —S(O)ₘNR^{1a}COₙR²,

### —S(O)ₘNR^{1a}CONR^{1c}R²,

### —OCONR^{1a}S(O)ₘR²,

### —OCONR^{1a}S(O)ₘNR^{1c}R²,

### —ONR^{1a}COₙR²

### —OCONR^{1c}R², or

### —ONR^{1a}CONR^{1c}R²

wherein
R^{1a} is a hydrogen atom;
R^{1b} is a hydrogen atom;
R^{1c} is a hydrogen atom, a C₁₋₆ alkyl group (preferably methyl) or a C₁₋₆ alkoxy group (preferably propoxy);
R² is
(1) a hydrogen atom,
(2) a C₁₋₁₀ alkyl group (preferably methyl, ethyl, propyl, butyl, isobutyl, pentyl, isopentyl, neopentyl, 1-ethylpropyl, 1-propylbutyl, 4-methylpentyl) optionally substituted by 1 to 3 substituents selected from
   (a) a C₆₋₁₄ aryl group (preferably phenyl) optionally substituted by 1 to 3 C₁₋₆ alkoxy groups (preferably methoxy),
   (b) a C₁₋₆ alkoxy group (preferably isopropoxy),
   (c) a C₁₋₆ alkoxy-carbonyl group (preferably ethoxycarbonyl),
   (d) a C₃₋₁₀ cycloalkyl group (preferably cyclopropyl, cyclohexyl),
   (e) a hydroxy group, and
   (f) a halogen atom (preferably fluorine atom);
(3) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(4) a C₃₋₁₀ cycloalkyl group (preferably cyclopropyl, cyclohexyl);
(5) an aromatic heterocyclic group (preferably furyl, thienyl, imidazolyl) optionally substituted by 1 to 3 C₁₋₆ alkyl groups (preferably methyl); or
(6) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy);
m is 2; and
n is 1 or 2, or
   (B) a 5- or 6-membered non-aromatic heterocyclic group containing NH [preferably oxooxadiazolinyl (preferably 5(4H)-oxo-1,2,4-oxadiazol-3-yl), 2,4-dioxothiazolidinyl (preferably 2,4-dioxothiazolidin-5-yl), 2,4-dioxoimidazolidinyl (preferably 2,4-dioxoimidazolidin-3-yl), 2-oxopiperazinyl (preferably 2-oxopiperazin-1-yl), 1,1-dioxido-3-oxothiadiazolidinyl (preferably 1,1-dioxido-3-oxo-1,2,5-thiadiazolidin-5-yl)] optionally substituted by 1 to 3 C₁₋₆ alkyl groups (preferably propyl, isopropyl).

### [Compound BB-2]

Compound BB-1 wherein

W is a group represented by

### —CONR^{1a}S(O)ₘR²,

### —CONR^{1a}S(O)ₘNR^{1c}R²,

### —S(O)ₘNR^{1a}COₙR², or

### —S(O)ₘNR^{1a}CONR^{1c}R²

wherein
R^{1a} is a hydrogen atom;
R^{1c} is a hydrogen atom, a C₁₋₆ alkyl group (preferably methyl) or a C₁₋₆ alkoxy group (preferably propoxy);
R² is
(1) a hydrogen atom,
(2) a C₁₋₁₀ alkyl group (preferably methyl, ethyl, propyl, butyl, isobutyl, pentyl, isopentyl, neopentyl, 1-propylbutyl, 4-methylpentyl) optionally substituted by 1 to 3 substituents selected from
   (a) a C₆₋₁₄ aryl group (preferably phenyl),
   (b) a C₁₋₆ alkoxy group (preferably isopropoxy),
   (c) a C₁₋₆ alkoxy-carbonyl group (preferably ethoxycarbonyl),
   (d) a C₃₋₁₀ cycloalkyl group (preferably cyclopropyl, cyclohexyl),
   (e) a hydroxy group, and
   (f) a halogen atom (preferably fluorine atom);
(3) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(4) a C₃₋₁₀ cycloalkyl group (preferably cyclopropyl, cyclohexyl);
(5) an aromatic heterocyclic group (preferably furyl, thienyl, imidazolyl) optionally substituted by 1 to 3 C₁₋₆ alkyl groups (preferably methyl); or
(6) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy);
m is 2; and
n is 1 or 2.

### [Compound BB-3]

Compound BB-1 wherein

X is

(1) a C₁₋₄ alkylene group (preferably —CH₂—, —CH₂CH₂—, —CH₂CH₂CH₂—; or
(2) a C₂₋₄ alkenylene group (preferably —CH═CH—, —CH═C(CH₃)—, —CH₂—CH═CH—); and

W is a group represented by

### —CONR^{1a}S(O)ₘR²,

### —CONR^{1a}S(O)ₘNR^{1c}R²,

### —S(O)ₘNR^{1a}COₙR², or

### —S(O)ₘNR^{1a}CONR^{1c}R²

wherein
R^{1a} is a hydrogen atom;
R^{1c} is a hydrogen atom, a C₁₋₆ alkyl group (preferably methyl) or a C₁₋₆ alkoxy group (preferably propoxy);
R² is
(1) a hydrogen atom,
(2) a C₁₋₁₀ alkyl group (preferably methyl, ethyl, propyl, butyl, isobutyl, pentyl, isopentyl, neopentyl, 1-propylbutyl, 4-methylpentyl) optionally substituted by 1 to 3 substituents selected from
   (a) a C₆₋₁₄ aryl group (preferably phenyl),
   (b) a C₁₋₆ alkoxy group (preferably isopropoxy),
   (c) a C₁₋₆ alkoxy-carbonyl group (preferably ethoxycarbonyl),
   (d) a C₃₋₁₀ cycloalkyl group (preferably cyclopropyl, cyclohexyl),
   (e) a hydroxy group, and
   (f) a halogen atom (preferably fluorine atom);
(3) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(4) a C₃₋₁₀ cycloalkyl group (preferably cyclopropyl, cyclohexyl);
(5) an aromatic heterocyclic group (preferably furyl, thienyl, imidazolyl) optionally substituted by 1 to 3 C₁₋₆ alkyl groups (preferably methyl); or
(6) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy);
m is 2; and
n is 1 or 2.

### [Compound CC]

Compound BB-1 wherein

X is

(1) a C₁₋₄ alkylene group (preferably —CH₂—, —CH₂CH₂—, —CH₂CH₂CH₂—; or
(2) a C₂₋₄ alkenylene group (preferably —CH═CH—, —CH═C(CH₃)—, —CH₂—CH═CH—); and

W is a group represented by

### —CONR^{1a}S(O)ₘR²

wherein
R^{1a} is a hydrogen atom;
R² is
(1) a C₁₋₁₀ alkyl group (preferably methyl, propyl, butyl, pentyl, 4-methylpentyl) optionally substituted by 1 to 3 substituents selected from
   (a) a C₆₋₁₄ aryl group (preferably phenyl), and
   (b) a C₃₋₁₀ cycloalkyl group (preferably cyclopropyl);
(2) a C₆₋₁₄ aryl group (preferably phenyl) optionally substituted by 1 to 3 substituents selected from
   (a) a halogen atom (preferably chlorine atom),
   (b) a C₁₋₆ alkyl group (preferably methyl, butyl) optionally substituted by 1 to 3 halogen atoms (preferably fluorine atom),
   (c) a C₁₋₆ alkoxy group (preferably methoxy), and
   (d) a hydroxy group;
(3) a C₃₋₁₀ cycloalkyl group (preferably cyclopropyl);
(4) an aromatic heterocyclic group (preferably furyl, thienyl, imidazolyl) optionally substituted by 1 to 3 C₁₋₆ alkyl groups (preferably methyl); or
(5) a non-aromatic heterocyclic group (preferably dihydrobenzofuranyl, morpholinyl, piperidinyl) optionally substituted by 1 to 3 substituents selected from
   (a) an oxo group,
   (b) a hydroxy group,
   (c) a C₁₋₆ alkyl group (preferably methyl), and
   (d) a C₁₋₃ alkylenedioxy group (preferably ethylenedioxy); and
m is 2.

### [Compound D]

(2E)-3-[1,3-dimethyl-5-(1H-pyrrolo[2,3-b]pyridin-1-yl)-1H-pyrazol-4-yl]-N-(pentylsulfonyl)acrylamide (Example 9),
(2E)-3-[5-(5-chloro-1H-indol-1-yl)-1,3-dimethyl-1H-pyrazol-4-yl]-N-(pentylsulfonyl)acrylamide (Example 27),
(2E)-3-[1,3-dimethyl-5-(1H-pyrrolo[2,3-b]pyridin-1-yl)-1H-pyrazol-4-yl]-N-[(4-methylphenyl)sulfonyl]acrylamide (Example 33),
(2E)-3-[5-(5-chloro-1H-indol-1-yl)-1,3-dimethyl-1H-pyrazol-4-yl]-N-[(pentylamino)sulfonyl]acrylamide (Example 62),
cyclopropylmethyl ({2-[5-(5-chloro-1H-indol-1-yl)-1,3-dimethyl-1H-pyrazol-4-yl]ethyl}sulfonyl)carbamate (Example 189),
butyl ({2-[5-(5-chloro-1H-pyrrolo[2,3-b]pyridin-1-yl)-1-methyl-3-(trifluoromethyl)-1H-pyrazol-4-yl]ethyl}sulfonyl)carbamate (Example 197),
(2E)-3-[1,3-dimethyl-5-(5-methyl-1H-pyrrolo[2,3-b]pyridin-1-yl)-1H-pyrazol-4-yl]-N-(pentylsulfonyl)acrylamide (Example 232),
(2E)-3-[5-(5-chloro-1H-pyrrolo[2,3-b]pyridin-1-yl)-1,3-dimethyl-1H-pyrazol-4-yl]-N-{[(cyclopropylmethyl)amino]sulfonyl}acrylamide (Example 264),
N-[(butylamino)carbonyl]-2-[5-(5-chloro-1H-pyrrolo[2,3-b]pyridin-1-yl)-3-cyclopropyl-1-methyl-1H-pyrazol-4-yl]ethanesulfonamide (Example 279),
(2E)-N-(butylsulfonyl)-3-[5-(5-chloro-1H-pyrrolo[2,3-b]pyridin-1-yl)-1,3-dimethyl-1H-pyrazol-4-yl]acrylamide (Example 283),
N-[(butylamino)carbonyl]-2-{1,3-dimethyl-5-[5-(trifluoromethyl)-1H-pyrrolo[2,3-b]pyridin-1-yl]-1H-pyrazol-4-yl}ethanesulfonamide (Example 294), or
butyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate (Example 295),
   or a salt thereof.

The salts of a compound represented by the formula (I′) and a compound represented by the formula (I) are preferably pharmacologically acceptable salts and, for example, salts with inorganic bases, salts with organic bases, salts with inorganic acids, salts with organic acids, salts with basic or acidic amino acids and the like can be mentioned.

Preferable examples of the salts with inorganic base include alkali metal salts such as sodium salt, potassium salt and the like; alkaline earth metal salts such as calcium salt, magnesium salt and the like; aluminum salt, ammonium salt and the like.

Preferable examples of the salt with organic base include a salt with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N′-dibenzylethylenediamine and the like.

Preferable examples of the salt with inorganic acid include a salt with hydrochloric acid, hydrobromic acid, nitric acid, sulfuric acid, phosphoric acid and the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include a salt with arginine, lysine, ornithine and the like.

Preferable examples of the salt with acidic amino acid include a salt with aspartic acid, glutamic acid and the like.

The prodrug of the compounds (I′) and (I) (hereinafter, to be referred to as compound (I)) is a compound which is converted to the compound (I) with a reaction due to an enzyme, gastric acid, etc. under the physiological condition in the living body, that is, a compound which is converted to the compound (I) by enzymatic oxidation, reduction, hydrolysis, etc.; a compound which is converted to the compound (I) by hydrolysis etc. due to gastric acid, and the like. A prodrug of the compound (I) may be a compound obtained by subjecting an amino group in the compound (I) to an acylation, alkylation or phosphorylation (e.g., a compound obtained by subjecting an amino group in the compound (I) to an eicosanoylation, alanylation, pentylaminocarbonylation, (5-methyl-2-oxo-1,3-dioxolen-4-yl)methoxycarbonylation, tetrahydrofuranylation, tetrahydropyranylation, pyrrolidylmethylation, pivaloyloxymethylation or tert-butylation); a compound obtained by subjecting a hydroxy group in the compound (I) to an acylation, alkylation, phosphorylation or boration (e.g., a compound obtained by subjecting an hydroxy group in the compound (I) to an acetylation, palmitoylation, propanoylation, pivaloylation, succinylation, fumarylation, alanylation, dimethylaminomethylcarbonylation, or tetrahydropyranylation); a compound obtained by subjecting a carboxyl group in the compound (I) to an esterification or amidation (e.g., a compound obtained by subjecting a carboxyl group in the compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any of these compounds can be produced from the compound (I) by a method known per se.

A prodrug of the compound (I) may be a compound that converts to the compound (I) under physiological conditions as described in Development of Pharmaceutical Products, vol. 7, Molecule Design, 163-198, Hirokawa Shoten (1990).

The compound (I) may be in the form of a crystal, and the crystal form of the crystal may be single or plural. The crystal can be produced by a crystallization method known per se. In the present specification, the melting point means that measured using, for example, a micromelting point apparatus (Yanaco, MP-500D or Buchi, B-545) or a DSC (differential scanning calorimetry) device (SEIKO, EXSTAR6000) [heating rate: 5° C./min] and the like.

In general, the melting points vary depending on the measurement apparatuses, the measurement conditions and the like. The crystal in the present specification may show different values from the melting point described in the present specification, as long as they are within a general error range.

The crystal of the compound (I) is superior in physicochemical properties (melting point, solubility, stability etc.) and biological properties (pharmacokinetics (absorption, distribution, metabolism, excretion), efficacy expression, etc.), and thus it is extremely useful as a medicament.

The compound (I) may be a solvate (e.g., hydrate) or a non-solvate, both of which are encompassed in the compound (I).

The compound (I) may be labeled with an isotope (e.g., ³H, ¹⁴C, ³⁵S, ¹²⁵I etc.) and the like. It is also encompassed in the compound (I).

Deuterium-converted compound wherein ¹H has been converted to ²H(D) are also encompassed in the compound (I).

The compound (I) or a prodrug thereof (hereinafter sometimes to be simply abbreviated as the compound of the present invention) shows low toxicity (e.g., acute toxicity, chronic toxicity, genetic toxicity, reproductive toxicity, cardiotoxicity, drug interaction, carcinogenicity), and can be used as it is or as a pharmaceutical composition in admixture with a commonly known pharmaceutically acceptable carrier etc., as an agent for the prophylaxis or treatment of the below-mentioned various disease, an insulin sensitizer and the like, in mammals (e.g., humans, mice, rats, rabbits, dogs, cats, bovines, horses, pigs, monkeys).

Here, as the pharmacologically acceptable carrier, various organic or inorganic carrier substances conventionally used as a preparation material can be used. They are incorporated as excipient, lubricant, binder and disintegrant for solid preparations; solvent, dissolution aids, suspending agent, isotonicity agent, buffer and soothing agent for liquid preparations and the like. Where necessary, preparation additives such as preservatives, antioxidants, coloring agents, sweetening agents and the like can be used.

As preferable examples of the excipient, lactose, sucrose, D-mannitol, D-sorbitol, starch, α-starch, dextrin, crystalline cellulose, low-substituted hydroxypropylcellulose, sodium carboxymethylcellulose, gum arabic, pullulan, light anhydrous silicic acid, synthetic aluminum silicate, magnesium alumino metasilicate and the like can be mentioned.

As preferable examples of the lubricant, magnesium stearate, calcium stearate, talc, colloidal silica and the like can be mentioned.

As preferable examples of the binder, α-starch, saccharose, gelatin, gum arabic, methylcellulose, carboxymethylcellulose, carboxymethylcellulose sodium, crystalline cellulose, sucrose, D-mannitol, trehalose, dextrin, pullulan, hydroxypropylcellulose, hydroxypropylmethylcellulose, polyvinylpyrrolidone and the like can be mentioned.

As preferable examples of the disintegrant, lactose, sucrose, starch, carboxymethylcellulose, carboxymethylcellulose calcium, croscarmellose sodium, carboxymethylstarch sodium, light anhydrous silicic acid, low-substituted hydroxypropylcellulose and the like can be mentioned.

As preferable examples of the solvent, water for injection, physiological brine, Ringer solution, alcohol, propylene glycol, polyethylene glycol, sesame oil, corn oil, olive oil, cottonseed oil and the like can be mentioned.

As preferable examples of the dissolution aids, polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like can be mentioned.

As preferable examples of the suspending agent, surfactants such as stearyltriethanolamine, sodium lauryl sulfate, laurylaminopropionic acid, lecithin, benzalkonium chloride, benzethonium chloride, glycerol monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; polysorbates, polyoxyethylene hydrogenated castor oil, and the like can be mentioned.

As preferable examples of the isotonicity agent, sodium chloride, glycerin, D-mannitol, D-sorbitol, glucose and the like can be mentioned.

As preferable examples of the buffer, buffers such as phosphate, acetate, carbonate, citrate and the like, and the like can be mentioned.

As preferable examples of the soothing agent, benzyl alcohol and the like can be mentioned.

As preferable examples of the preservative, p-oxybenzoates, chlorobutanol, benzyl alcohol, phenethyl alcohol, dehydroacetic acid, sorbic acid and the like can be mentioned.

As preferable examples of the antioxidant, sulfite, ascorbate and the like can be mentioned.

As preferable examples of the coloring agent, water-soluble food tar colors (e.g., food colors such as Food Red Nos. 2 and 3, Food Yellow Nos. 4 and 5, Food Blue Nos. 1 and 2 and the like), water insoluble lake dye (e.g., aluminum salts of the aforementioned water-soluble food tar colors), natural dyes (e.g., β-carotene, chlorophyll, red iron oxide) and the like can be mentioned.

As preferable examples of the sweetening agent, saccharin sodium, dipotassium glycyrrhizinate, aspartame, stevia and the like can be mentioned.

The dosage form of the aforementioned pharmaceutical composition is, for example, an oral agent such as tablets (inclusive of sugar-coated tablets, film-coated tablets, sublingual tablets and orally disintegrable tablets), capsules (inclusive of soft capsules and microcapsules), granules, powders, troches, syrups, emulsions, suspensions, films (e.g., orally disintegrable film) and the like; a parenteral agent such as injections (e.g., subcutaneous injections, intravenous injections, intramuscular injections, intraperitoneal injections, drip infusions), external agents (e.g., transdermal preparations, ointments), suppositories (e.g., rectal suppositories, vaginal suppositories), pellets, nasal preparations, pulmonary preparations (inhalations), ophthalmic preparations and the like, and the like. These may be administered safely via an oral or parenteral (e.g., topical, rectal, intravenous administrations etc.) route.

These preparations may be controlled-release preparations (e.g., sustained-release microcapsule) such as immediate-release preparation, sustained-release preparation and the like.

The pharmaceutical composition can be produced by a method conventionally used in the preparation technical field, such as a method described in the Japanese Pharmacopoeia and the like.

While the content of the compound of the present invention in the pharmaceutical composition varies depending on the dosage form, the dose of the compound of the present invention and the like, it is, for example, about 0.1 to 100 wt %.

The compound of the present invention has a hypoglycemic action, a hypolipidemic action, an insulin sensitizing action, an insulin sensitivity enhancing action and a peroxisome growth responsive receptor (PPAR)γ (GenBank Accession No. L40904) agonist (activation) action. Here, PPARγ may form a heterodimer receptor with any of retinoid X receptor (RXR)α (GenBank Accession No. X52773), RXRβ (GenBank Accession No. M84820) and RXRγ (GenBank Accession No. U38480).

The compound of the present invention particularly has a selective partial agonist (partial agonist) action on PPARγ.

A selective partial agonist for PPARγ has been reported to be unaccompanied by side effects such as body weight gain, adipocyte accumulation, cardiac hypertrophy and the like, as compared to a full agonist for PPARγ (e.g., thiazolidinedione compound) (Molecular Endocrinology, vol. 17, NO. 4, page 662, 2003). Therefore, the compound of the present invention is useful as a hypoglycemic agent unaccompanied by side effects such as body weight gain, adipocyte accumulation, cardiac hypertrophy and the like, as compared to a full agonist for PPARγ.

The compound of the present invention can be used, for example, as an agent for the prophylaxis or treatment of diabetes (e.g., type-1 diabetes, type-2 diabetes, gestational diabetes, obesity diabetes); an agent for the prophylaxis or treatment of hyperlipidemia (e.g., hypertriglyceridemia, hypercholesterolemia, hypo-HDL-emia, postprandial hyperlipidemia); insulin sensitizer; an agent for enhancing insulin sensitivity; an agent for the prophylaxis or treatment of impaired glucose tolerance [IGT (Impaired Glucose Tolerance)]; and an agent for preventing progress of impaired glucose tolerance into diabetes.

For diagnostic criteria of diabetes, Japan Diabetes Society reported new diagnostic criteria.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) and WHO reported new diagnostic criteria of diabetes.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports of ADA and WHO, impaired glucose tolerance is a condition showing a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 100 mg/dl and less than 126 mg/dl is called IFG (Impaired Fasting Glucose). On the other hand, WHO defines the IFG (Impaired Fasting Glucose) to be a condition showing a fasting blood glucose level (glucose concentration of intravenous plasma) of not less than 110 mg/dl and less than 126 mg/dl, and calls it IFG (Impaired Fasting Glycaemia).

The compound of the present invention can be also used as an agent for the prophylaxis or treatment of diabetes, borderline type, impaired glucose tolerance, IFG (Impaired Fasting Glucose) and IFG (Impaired Fasting Glycaemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycaemia) into diabetes.

The compound of the present invention can also be used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], obesity, osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, hypertension, polycystic ovary syndrome, kidney disease (e.g., diabetic nephropathy, glomerular nephritis, glomerulosclerosis, nephrotic syndrome, hypertensive nephrosclerosis, end stage kidney disease), muscular dystrophy, myocardial infarction, angina pectoris, cerebrovascular accident (e.g., cerebral infarction, cerebral apoplexy), insulin resistance syndrome, Syndrome X, metabolic syndrome (pathology having three or more selected from hypertriglyceridemia (TG), hypoHDL cholesterolemia (HDL-C), hypertension, abdomen overweight and impaired glucose tolerance), hyperinsulinemia, hyperinsulinemia-induced sensory disorder, tumor (e.g., leukemia, breast cancer, prostate cancer, skin cancer), irritable bowel syndrome, acute or chronic diarrhea, inflammatory diseases (e.g., arteriosclerosis (e.g., atherosclerosis), chronic rheumatoid arthritis, spondylitis deformans, osteoarthritis, lumbago, gout, postoperative or traumatic inflammation, swelling, neuralgia, pharyngolaryngitis, cystitis, hepatitis (inclusive of nonalcoholic steatohepatitis), pneumonia, pancreatitis, inflammatory bowel disease, ulcerative colitis, chronic obstructive pulmonary disease (COPD)), visceral obesity syndrome, leg ulcer, sepsis, psoriasis and the like.

In addition, the compound of the present invention can also be used for ameliorating the conditions such as abdominal pain, nausea, vomiting, discomfort in the upper abdomen and the like, which are associated with peptic ulcer, acute or chronic gastritis, biliary dyskinesia, cholecystitis and the like, and the like.

The compound of the present invention can also be used as an agent for the prophylaxis or treatment of inflammatory disease involving TNF-αc. Here, the inflammatory disease involving TNF-α is an inflammatory disease developed by the presence of TNF-α, which can be treated via a TNF-α inhibitory effect. As such inflammatory disease, for example, diabetic complications (e.g., retinopathy, nephropathy, neuropathy, macroangiopathy), chronic rheumatoid arthritis, spondylitis deformans, osteoarthritis, lumbago, gout, postoperative or traumatic inflammation, swelling, neuralgia, pharyngolaryngitis, cystitis, hepatitis, pneumonia, stomach mucous membrane injury (including stomach mucous membrane injury caused by aspirin) and the like can be mentioned.

The compound of the present invention has an apoptosis inhibitory action and can also be used as an agent for the prophylaxis or treatment of diseases involving promotion of apoptosis. As the disease involving promotion of apoptosis, for example, viral diseases (e.g., AIDS, fulminant hepatitis), neurodegenerative diseases (e.g., Alzheimer's disease, Parkinson's syndrome, amyotrophic lateral sclerosis, pigmentosa, cerebellar degeneration), myelodysplasia (e.g., aplastic anemia), ischemic diseases (e.g., cardiac infarction, cerebral apoplexy), hepatic diseases (e.g., alcoholic hepatitis, hepatitis B, hepatitis C), joint-diseases (e.g., osteoarthritis), atherosclerosis and the like can be mentioned.

The compound of the present invention can also be used for reduction of visceral fat, inhibition of visceral fat accumulation, glycometabolism improvement, lipometabolism improvement, insulin resistance improvement, oxidized LDL production inhibition, lipoprotein metabolism improvement, coronary metabolism improvement, prophylaxis or treatment of cardiovascular complications, prophylaxis or treatment of heart failure complications, decrease of blood remnant, prophylaxis or treatment of anovulation, prophylaxis or treatment of hirsutism, prophylaxis or treatment of hyperandrogenemia and the like.

The compound of the present invention can also be used as secondary prevention and suppression of progression of the above-mentioned various diseases (e.g., cardiovascular event such as cardiac infarction and the like).

While the dose of the compound of the present invention varies depending on the administration subject, administration route, target disease, condition and the like, for example, it is generally about 0.005 to 50 mg/kg body weight, preferably 0.01 to 2 mg/kg body weight, more preferably 0.025 to 0.5 mg/kg body weight, for oral administration to adult diabetic patients, which is desirably administered in one to three portions a day.

The compound of the present invention can be used in combination with pharmaceutical agents (hereinafter to be abbreviated as combination drug) such as therapeutic agents for diabetes, therapeutic agents for diabetic complications, therapeutic agents for hyperlipidemia, antihypertensive agents, antiobesity agents, diuretics, chemotherapeutic agents, immunotherapeutic agents, antithrombotic agents, therapeutic agents for osteoporosis, antidementia agents, erectile dysfunction ameliorating agents, therapeutic agents for urinary incontinence or pollakiuria, therapeutic agents for dysuria and the like. These combination drugs may be low-molecular-weight compounds, high-molecular-weight proteins, polypeptides, antibodies or nucleic acids (including antisense nucleic acid, siRNA, shRNA), vaccines and the like.

The administration time of the compound of the present invention and the combination drug is not restricted, and these can be administered to an administration subject simultaneously, or may be administered at staggered times.

As the administration mode of the compound of the present invention and the combination drug, the following methods can be mentioned: (1) The compound of the present invention and the combination drug are simultaneously formulated to give a single preparation which is administered. (2) The compound of the present invention and the combination drug are separately formulated to give two kinds of preparations which are administered simultaneously by the same administration route. (3) The compound of the present invention and the combination drug are separately formulated to give two kinds of preparations which are administered by the same administration route at staggered times. (4) The compound of the present invention and the combination drug are separately formulated to give two kinds of preparations which are administered simultaneously by the different administration routes. (5) The compound of the present invention and the combination drug are separately formulated to give two kinds of preparations which are administered by the different administration routes at staggered times (for example, the compound of the present invention and the combination drug are administered in this order, or in the reverse order), and the like.

The dose of the combination drug can be appropriately determined based on the dose employed clinically. The mixing ratio of the compound of the present invention and a combination drug can be appropriately determined depending on the administration subject, administration route, target disease, symptom, combination and the like. When the administration subject is human, for example, a combination drug can be used in 0.01 to 100 parts by weight relative to 1 part by weight of the compound of the present invention.

Examples of the therapeutic agents for diabetes include insulin preparations (e.g., animal insulin preparations extracted from pancreas of bovine or swine; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (preferably hydrochloride), rosiglitazone or a salt thereof (preferably maleate), Tesaglitazar, Ragaglitazar, Muraglitazar, Edaglitazone, Metaglidasen, Naveglitazar, AMG-131, THR-0921), α-glucosidase inhibitors (e.g., voglibose, acarbose, miglitol, emiglitate), biguanides (e.g., metformin, buformin or a salt thereof (e.g., hydrochloride, fumarate, succinate)), insulin secretagogues [sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or a calcium salt hydrate thereof], dipeptidyl peptidase IV inhibitors (e.g., Alogliptin or a salt thereof (preferably benzoate), Vildagliptin, Sitagliptin, Saxagliptin, T-6666, TS-021), β3 agonists (e.g., AJ-9677), GPR40 agonists, GLP-1 receptor agonists [e.g., GLP-1, GLP-1MR agent, N,N-2211, AC-2993 (exendin-4), BIM-51077, Aib(8,35)hGLP-1(7,37)NH₂, CJC-1131], amylin agonists (e.g., pramlintide), phosphotyrosine phosphatase inhibitors (e.g., sodium vanadate), gluconeogenesis inhibitors (e.g., glycogen phosphorylase inhibitors, glucose-6-phosphatase inhibitors, glucagon antagonists), SGLUT (sodium-glucose cotransporter) inhibitors (e.g., T-1095), 11β-hydroxysteroid dehydrogenase inhibitors (e.g., BVT-3498), adiponectin or agonist thereof, IKK inhibitors (e.g., AS-2868), leptin resistance improving drugs, somatostatin receptor agonists, glucokinase activators (e.g., Ro-28-1675), GIP (Glucose-dependent insulinotropic peptide) and the like.

Examples of the therapeutic agents for diabetic complications include aldose reductase inhibitors (e.g., Tolrestat, Epalrestat, Zenarestat, Zopolrestat, Minalrestat, Fidarestat, CT-112, ranirestat (AS-3201)), neurotrophic factors and increasing drugs thereof (e.g., NGF, NT-3, BDNF, neurotrophin production-secretion promoters (e.g., 4-(4-chlorophenyl)-2-(2-methyl-1-imidazolyl)-5-oxazolepropanol, 4-(4-chlorophenyl)-2-(2-methyl-1-imidazolyl)-5-oxazolebutanol, 4-(4-chlorophenyl)-5-[3-(1-imidazolyl)propyl]-2-(2-methyl-1-imidazolyl)oxazole, 4-(4-chlorophenyl)-2-(2-methyl-1-imidazolyl)-5-oxazolepentanol, 4-(4-chlorophenyl)-5-[4-(1-imidazolyl)butyl]-2-(2-methyl-1-imidazolyl)oxazole, 3-[3-[4-(4-chlorophenyl)-2-(2-methyl-1-imidazolyl)-5-oxazolyl]propyl]-1-methyl-2,4-imidazolidinedione, 4-(4-chlorophenyl)-5-[3-(2-methoxyphenoxy)propyl]-2-(2-methyl-1-imidazolyl)oxazole, 4-(4-chlorophenyl)-5-[3-(3-methoxyphenoxy)propyl]-2-(2-methyl-1-imidazolyl)oxazole, 4-(4-chlorophenyl)-5-[3-(4-methoxyphenoxy)propyl]-2-(2-methyl-1-imidazolyl)oxazole, 4-(4-chlorophenyl)-2-(2-methyl-1-imidazolyl)-5-[3-(2-methylphenoxy)propyl]oxazole, diethyl [4-({(2E)-3-[5-(4-fluorophenyl)-1-methyl-1H-pyrazol-4-yl]prop-2-enoyl}amino)benzyl]phosphonate, (2E)-N-{4-[(2,4-dioxo-1,3-thiazolidin-5-yl)methyl]phenyl}-3-[5-(4-fluorophenyl)-1-methyl-1H-pyrazol-4-yl]acrylamide, (2E)-3-[5-(4-fluorophenyl)-1-methyl-1H-pyrazol-4-yl]-N-[4-(1H-imidazol-1-ylmethyl)phenyl]acrylamide, (2E)-3-[5-(4-fluorophenyl)-1-methyl-1H-pyrazol-4-yl]-N-[4-(1H-pyrazol-1-ylmethyl)phenyl]acrylamide, diethyl [4-({(2E)-3-[1-methyl-5-(2-thienyl)-1H-pyrazol-4-yl]prop-2-enoyl}amino)benzyl]phosphonate, (2E)-3-[5-(4-fluorophenyl)-1-methyl-1H-pyrazol-4-yl]-N-{4-[(3-methyl-2,4-dioxo-1,3-thiazolidin-5-yl)methyl]phenyl}acrylamide, (2E)-N-[4-(1H-benzimidazol-1-ylmethyl)phenyl]-3-[5-(4-fluorophenyl)-1-methyl-1H-pyrazol-4-yl]acrylamide, (2E)-3-[5-(4-fluorophenyl)-1-methyl-1H-pyrazol-4-yl]-N-{4-[(methylsulfonyl)methyl]phenyl}acrylamide, (2E)-3-[5-(4-fluorophenyl)-1-methyl-1H-pyrazol-4-yl]-N-{4-[hydroxy(2-pyridinyl)methyl]phenyl}acrylamide, (2E)-3-[5-(4-fluorophenyl)-1-methyl-1H-pyrazol-4-yl]-N-[4-(4-morpholinylmethyl)phenyl]acrylamide, (2E)-N-{4-[(ethylsulfonyl)methyl]phenyl}-3-[5-(4-fluorophenyl)-1-methyl-1H-pyrazol-4-yl]acrylamide), PKC inhibitors (e.g., ruboxistaurin mesylate)), AGE inhibitors (e.g., ALT946, pimagedine, N-phenacylthiazolium bromide (ALT-766), EXO-226, Pyridorin, Pyridoxamine), active oxygen scavengers (e.g., thioctic acid), cerebral vasodilators (e.g., tiapuride, mexiletine), somatostatin receptor agonist (e.g., BIM23190), apoptosis signal regulating kinase-1 (ASK-1) inhibitors and the like.

Examples of the hyperlipidemia therapeutic agents include HMG-CoA reductase inhibitors (e.g., cerivastatin, pravastatin, simvastatin, lovastatin, atorvastatin, fluvastatin, itavastatin, rosuvastatin, pitavastatin or a salt thereof (e.g., sodium salt, calcium salt)), squalene synthase inhibitors (e.g., lapaquistat or a salt thereof (preferably acetate)), fibrate compounds (e.g., bezafibrate, clofibrate, simfibrate, clinofibrate), ACAT inhibitors (e.g., Avasimibe, Eflucimibe), anion exchange resins (e.g., colestyramine), probucol, nicotinic acid drugs (e.g., nicomol, niceritrol), ethyl icosapentate, phytosterols (e.g., soysterol, γ-oryzanol) and the like.

Examples of the antihypertensive agents include angiotensin converting enzyme inhibitors (e.g., captopril, enalapril, delapril), angiotensin II antagonists (e.g., candesartan cilexetil, losartan, eprosartan, valsartan, telmisartan, irbesartan, olmesartan medoxomil, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid), calcium channel blockers (e.g., manidipine, nifedipine, nicardipine, amlodipine, efonidipine), potassium channel openers (e.g., levcromakalim, L-27152, AL0671, NIP-121), clonidine and the like.

Examples of the antiobesity agents include antiobesity agents acting on the central nervous system (e.g., dexfenfluramine, fenfluramine, phentermine, sibutramine, amfepramone, dexamphetamine, mazindol, phenylpropanolamine, clobenzorex; MCH receptor antagonists (e.g., SB-568849; SNAP-7941; compounds described in WO01/82925 and WO01/87834); neuropeptide Y antagonists (e.g., CP-422935); cannabinoid receptor antagonists (e.g., SR-141716, SR-147778); ghrelin antagonists; 11β-hydroxysteroid dehydrogenase inhibitors (e.g., BVT-3498)), pancreatic lipase inhibitors (e.g., orlistat, cetilistat (ATL-962)), β3 agonists (e.g., AJ-9677), peptide anorexiants (e.g., leptin, CNTF (Ciliary Neurotropic Factor)), cholecystokinin agonists (e.g., lintitript, FPL-15849), feeding deterrents (e.g., P-57) and the like.

Examples of the diuretics include xanthine derivatives (e.g., sodium salicylate and theobromine, calcium salicylate and theobromine), thiazide preparations (e.g., ethiazide, cyclopenthiazide, trichloromethiazide, hydrochlorothiazide, hydroflumethiazide, bentylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlortalidone, mefruside, indapamide), azosemide, isosorbide, etacrynic acid, piretanide, bumetanide, furosemide and the like.

Examples of the chemotherapeutic agents include alkylating agents (e.g., cyclophosphamide, ifosfamide), metabolic antagonists (e.g., methotrexate, 5-fluorouracil and a derivative thereof), antitumor antibiotics (e.g., mitomycin, adriamycin), plant-derived antitumor agent (e.g., vincristine, vindesine, Taxol), cisplatin, carboplatin, etoposide and the like. Of these, Furtulon or NeoFurtulon, which are 5-fluorouracil derivatives, and the like are preferable.

Examples of the immunotherapeutic agents include microorganism or bacterial components (e.g., muramyl dipeptide derivative, Picibanil), polysaccharides having immunity potentiating activity (e.g., lentinan, schizophyllan, krestin), cytokines obtained by genetic engineering techniques (e.g., interferon, interleukin (IL)), colony stimulating factors (e.g., granulocyte colony stimulating factor, erythropoietin) and the like, with preference given to interleukins such as IL-1, IL-2, IL-12 and the like.

Examples of the antithrombotic agents include heparin (e.g., heparin sodium, heparin calcium, dalteparin sodium), warfarin (e.g., warfarin potassium), anti-thrombin drugs (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like.

Examples of the therapeutic agents for osteoporosis include alfacalcidol, calcitriol, elcatonin, calcitonin salmon, estriol, ipriflavone, risedronate disodium, pamidronate disodium, alendronate sodium hydrate, incadronate disodium and the like.

Examples of the antidementia agents include tacrine, donepezil, rivastigmine, galanthamine and the like.

Examples of the erectile dysfunction ameliorating agents include apomorphine, sildenafil citrate and the like.

Examples of the therapeutic agents for urinary incontinence or pollakiuria include flavoxate hydrochloride, oxybutynin hydrochloride, propiverine hydrochloride and the like.

Examples of the therapeutic agents for dysuria include acetylcholine esterase inhibitors (e.g., distigmine) and the like.

Examples of the combination drugs include drugs having a cachexia-ameliorating action established in animal models and clinical situations, such as cyclooxygenase inhibitors (e.g., indomethacin), progesterone derivatives (e.g., megestrol acetate), glucosteroids (e.g., dexamethasone), metoclopramide agents, tetrahydrocannabinol agents, fat metabolism improving agents (e.g., eicosapentanoic acid), growth hormones, IGF-1, or antibodies to a cachexia-inducing factor such as TNF-α, LIF, IL-6, oncostatin M and the like.

As the combination drugs, nerve regeneration promoting drugs (e.g., Y-128, VX853, prosaptide), antidepressants (e.g., desipramine, amitriptyline, imipramine), antiepileptics (e.g., lamotrigine), antiarrhythmic agents (e.g., mexiletine), acetylcholine receptor ligands (e.g., ABT-594), endothelin receptor antagonists (e.g., ABT-627), monoamine uptake inhibitors (e.g., tramadol), narcotic analgesics (e.g., morphine), GABA receptor agonists (e.g., gabapentin), α2 receptor agonists (e.g., clonidine), local analgesics (e.g., capsaicin), antianxiety drugs (e.g., benzothiazepines), dopamine receptor agonists (e.g., apomorphine), midazolam, ketoconazole and the like can also be mentioned.

The combination drug is preferably an insulin preparation, an insulin sensitizer, an α-glucosidase inhibitor, biguanide, insulin secretagogue (preferably sulfonylurea) and the like.

The above-mentioned combination drugs may be used in a mixture of two or more kinds thereof at an appropriate ratio.

When the compound of the present invention is used in combination with a combination drug, the dose of each agent can be reduced within a safe range in consideration of the side effects thereof. Particularly, the doses of insulin sensitizers, insulin secretagogues and biguanides can be reduced from generally dose levels. Therefore, the side effects possibly caused by these agents can be safely prevented. In addition, the doses of the therapeutic agents for diabetic complications, the therapeutic agents for hyperlipidemia and the antihypertensive agents can be reduced, and as a result, the side effects possibly caused by these agents can be effectively prevented.

The production method of the compound of the present invention is explained in the following.

Compound (I) can be produced according to a method known per se, for example, according to the following Method A1, Method A2, Method B to Method G, Method H1, Method H2, Method I to Method N, Method O1, Method O2, Method P to Method R, Method S1, Method S2, Method AA to Method AL, Method AU and Method AW or a method analogous thereto.

In each production method, starting material compounds may be used in the form of a salt. As such salts, those similar to the salts of a compound represented by the formula (I) can be used.

Compound (I-1), which is compound (I) wherein W is —CONR^{1a}S(O)ₘR² wherein each symbol is as defined, is produced, for example, according to the following Method A1.

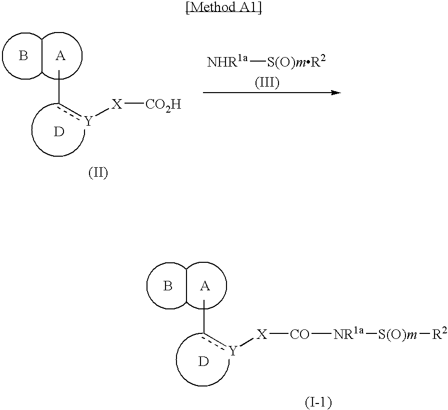

wherein each symbol is as defined above.

In this method, compound (I-1) can be produced by subjecting compound (II) to a condensation reaction. This reaction is carried out according a method known per se, for example, method of directly condensing compound (II) with compound (III), or method of reacting a reactive derivative of compound (II) with compound (III), and the like. As the reactive derivative of compound (II), for example, acid halides (e.g., acid chlorides, acid bromides), imidazolide, mixed acid anhydrides (e.g., anhydrides with methyl carbonate, ethyl carbonate or isobutyl carbonate, etc.) and the like can be mentioned.

The method of directly condensing compound (II) with compound (III) is carried out in the presence of a condensing agent, in a solvent that does not adversely influence the reaction.

As the condensing agent, a condensing agent known in the field, for example, carbodiimide condensing reagents such as dicyclohexylcarbodiimide, diisopropylcarbodiimide, N-[3-(dimethylamino)propyl]-N′-ethylcarbodiimide and a hydrochloride thereof and the like; phosphoric acid condensing reagents such as diethyl cyanophosphate, diphenyl azidophosphate and the like; 2-methyl-6-nitrobenzoic anhydride, N,N′-carbonyldiimidazole, 2-chloro-1,3-dimethylimidazolium tetrafluoroborate and the like can be mentioned.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, propionitrile, ethyl acetate, pyridine, water and the like can be mentioned. These solvents may be used in a mixture at an appropriate ratio. These solvents may be used in a mixture at an appropriate ratio.

The amount of compound (III) to be used is generally 0.1 to 10 mol, preferably 0.3 to 3 mol, per 1 mol of compound (II).

The amount of the condensing agent to be used is generally 0.1 to 10 mol, preferably 0.3 to 5 mol, per 1 mol of compound (II).

When a carbodiimide condensing reagent or 2-methyl-6-nitrobenzoic anhydride is used as a condensing agent, if necessary, the reaction efficiency can be improved by using a suitable condensation promoter (e.g., 1-hydroxy-7-azabenzotriazole, 1-hydroxybenzotriazole, N-hydroxysuccinimide, N-hydroxyphthalimide, 4-dimethylaminopyridine etc.). When a phosphoric acid condensing reagent or 2-methyl-6-nitrobenzoic anhydride is used as a condensing agent, generally, the reaction efficiency can be improved by adding an organic amine base such as triethylamine, diisopropylethylamine and the like.

The amount of the condensation promoter and organic amine base to be used is generally 0.1 to 10 mol, preferably 0.3 to 5 mol, per 1 mol of compound (II), respectively.

The reaction temperature is generally −30° C. to 100° C.

The reaction time is generally 0.1 to 100 hr.

When an acid halide is used as a reactive derivative of compound (II), the reaction is carried out by reacting compound (II) with a halogenating agent in a solvent that does not adversely influence the reaction, and reacting the resulting compound with compound (III) in the presence of a base.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like; amides such as N,N-dimethylformamide, N,N-dimethylacetamide and the like; acetonitrile, ethyl acetate, water and the like can be mentioned. These solvents may be used in a mixture at an appropriate ratio.

As the halogenating agent, for example, thionyl chloride, oxalyl chloride, phosphoryl chloride and the like can be mentioned.

As the base, for example, amines such as triethylamine, N,N-diisopropylethylamine, N-methylmorpholine, N,N-dimethylaniline, 1,8-diazabicyclo[5.4.0]undec-7-ene, pyridine and the like; alkali metal salts such as sodium hydrogencarbonate, sodium carbonate, potassium carbonate and the like, and the like can be mentioned.

The amount of compound (III) to be used is generally 0.1 to 10 mol, preferably 0.3 to 3 mol, per 1 mol of compound (II).

The amount of the halogenating agent to be used is generally 1 to 50 mol, preferably 1 to 10 mol, per 1 mol of compound (II).

The amount of the base to be used is generally 1 to 20 mol, preferably 1 to 5 mol, per 1 mol of compound (II).

The reaction temperature is generally −30° C. to 100° C.

The reaction time is generally 0.1 to 30 hr.

When a mixed acid anhydride is used as a reactive derivative of compound (II), the reaction is carried out by reacting compound (II) with a chlorocarbonate in the presence of a base, and reacting the resulting compound with compound (III).

As the chlorocarbonate, for example, methyl chlorocarbonate, ethyl chlorocarbonate, isobutyl chlorocarbonate and the like can be mentioned.

As the base, for example, amines such as triethylamine, N,N-diisopropylethylamine, N-methylmorpholine, N,N-dimethylaniline and the like; alkali metal salts such as sodium hydrogencarbonate, sodium carbonate, potassium carbonate and the like, and the like can be mentioned.

The amount of compound (III) to be used is generally 0.1 to 10 mol, preferably 0.3 to 3 mol, per 1 mol of compound (II).

The amount of the chlorocarbonate to be used is generally 1 to 10 mol, preferably 1 to 5 mol, per 1 mol of compound (II).

The amount of the base to be used is generally 1 to 20 mol, preferably 1 to 5 mol, per 1 mol of compound (II).

The reaction temperature is generally −30° C. to 100° C.

The reaction time is generally 0.1 to 30 hr.

When an imidazolide is used as a reactive derivative of compound (II), the reaction is carried out by reacting compound (II) with N,N′-carbonyldiimidazole, and reacting the resulting compound with compound (III) in the presence of a base.

As the base, those similar to the base used for the aforementioned reaction using an acid halide can be mentioned.

The amount of the compound (III) to be used is generally 0.1 to 10 mol, preferably 0.3 to 3 mol, per 1 mol of compound (II).

The amount of the N,N′-carbonyldiimidazole to be used is generally 1 to 10 mol, preferably 1 to 5 mol, per 1 mol of compound (II).

The amount of the base to be used is generally 1 to 20 mol, preferably 1 to 5 mol, per 1 mol of compound (II).

The reaction temperature is generally −30° C. to 100° C.

The reaction time is generally 0.1 to 30 hr.

Compound (II) can be produced, for example, according to the below-mentioned Method T1 to Method T5, Step 1 of Method N or a method analogous thereto. Compound (III) can be produced according to a method known per se.

The alkali metal salt (I-1b) of compound (I-1a), which is compound (I) wherein W is —CONR^{1a}S(O)ₘR² wherein R^{1a} is a hydrogen atom and the other symbols are as defined above, is produced, for example, according to the following Method A2.

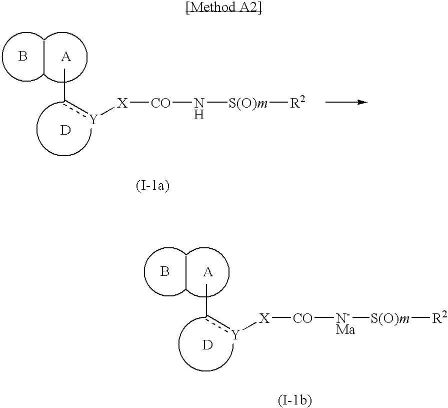

wherein Ma is an alkali metal, and the other symbols are as defined above.

As the alkali metal for Ma, sodium, potassium and the like can be mentioned.

In this method, compound (I-1b) can be produced by reacting compound (I-1a) with a base. This reaction is carried out in the presence of a base, in a water-containing solvent, according to a method known per se.

As the base, for example, alkali metal carbonates such as potassium hydrogencarbonate, sodium hydrogencarbonate and the like, and the like can be mentioned.

The amount of the base to be used is generally 1 to 2 mol, per 1 mol of compound (I-1a).

As the water-containing solvent, for example, a mixed solvent of water and 1 or more solvents selected from alcohols such as methanol, ethanol and the like; ethers such as tetrahydrofuran, dioxane, diethyl ether and the like; dimethyl sulfoxide, acetone and the like, and the like can be mentioned.

The reaction temperature is generally −30 to 150° C., preferably −10 to 50° C.

The reaction time is generally 0.1 to 20 hr.

Compound (I-1a) can be produced, for example, according to the above-mentioned Method A1, the below-mentioned Method AI, Method AJ, Method AL or a method analogous thereto.

Compound (I-2a), which is compound (I) wherein W is —CONR^{1a}S(O)ₘNR^{1c}R² wherein m is 2 and the other symbols are as defined above, is produced, for example, according to the following Method B.

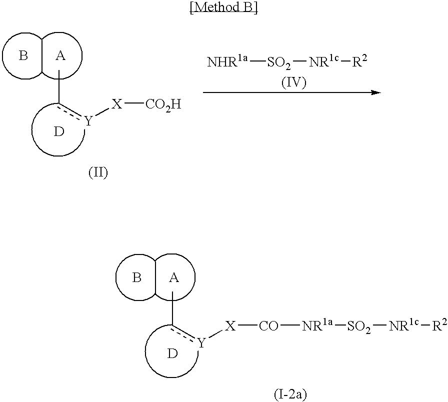

wherein each symbol is as defined above.

In this method, compound (I-2a) can be produced by reacting compound (II) with compound (IV). This reaction is carried out in the same manner as in the condensation reaction described in the aforementioned Method A1.

Compound (IV) can be produced, for example, according to the below-mentioned Method AT or a method analogous thereto.

Compound (I-3), which is compound (I) wherein W is —OCONR^{1a}S(O)ₘR² wherein each symbol is as defined above, is produced, for example, according to the following Method C or Method D.

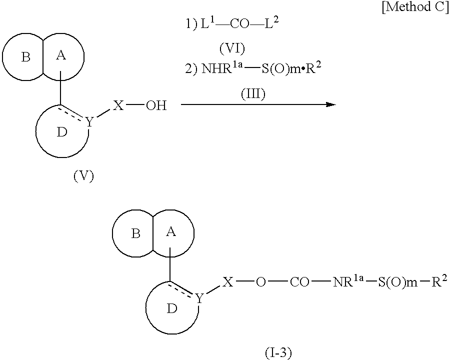

wherein L¹ and L² are independently a leaving group, and the other symbols are as defined above.

As the leaving group L¹ or L², for example, a hydroxy group, a halogen atom, a imidazolyl group, a succinimidooxy group, —OSO₂R³ wherein R³ is a C₁₋₄ alkyl group (preferably methyl), a C₆₋₁₀ aryl group optionally substituted by C₁₋₄ alkyl group(s) (preferably tolyl), and the like can be mentioned.

As compound (VI), for example, N,N′-carbonyldiimidazole, diphosgene, triphosgene and the like can be mentioned.

In this method, compound (I-3) can be produced from compound (V). This reaction is carried out according to a method known per se, for example, by reacting compound (V) with compound (VI) in a solvent that does not adversely influence the reaction, at −10° C. to 100° C. for 0.5 to 10 hr, and reacting the obtained compound with compound (III) in a solvent that does not adversely influence the reaction, at −10° C. to 100° C. for 0.5 to 50 hr.

This reaction may be carried out in the presence of 1 to 5 mol of a base, per 1 mol of compound (V).

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned. These bases may be used in a mixture at an appropriate ratio.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, pyridine, water and the like can be mentioned. These solvents may be used in a mixture at an appropriate ratio.

The amount of compound (VI) to be used is generally 1 to 10 mol, preferably 1 to 5 mol, per 1 mol of compound (V).

The amount of compound (III) to be used is generally 1 to 10 mol, preferably 1 to 5 mol, per 1 mol of compound (V).

Compound (V) can be produced, for example, according to the below-mentioned Method U1 or Method U2 or a method analogous thereto. Compound (VI) can be produced according to a method known per se.

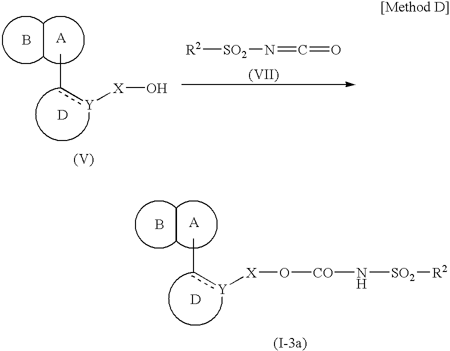

wherein each symbol is as defined above.

In this method, compound (I-3a), which is compound (I-3) wherein R^{1a} is a hydrogen atom and m is 2, can be produced by reacting compound (V) with compound (VII). This reaction is carried out in a solvent that does not adversely influence the reaction.

This reaction may be carried out in the presence of 1 to 5 mol of a base, per 1 mol of compound (V).

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned. These bases may be used in a mixture at an appropriate ratio.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, pyridine, water and the like can be mentioned. These solvents may be used in a mixture at an appropriate ratio.

The amount of compound (VII) to be used is generally 1 to 10 mol, preferably 1 to 5 mol, per 1 mol of compound (V).

The reaction temperature is generally −30° C. to 100° C.

The reaction time is generally 0.5 to 30 hr.

Compound (VII) can be produced according to a method known per se.

Compound (I-4), which is compound (I) wherein W is —OCONR^{1c}R² wherein each symbol is as defined above, is produced, for example, according to the following Method E or Method F.

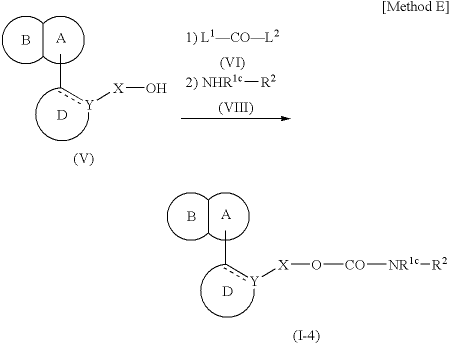

wherein each symbol is as defined above.

In this method, compound (I-4) can be produced from compound (V). This reaction is carried out according to a method known per se, for example, by reacting compound (V) with compound (VI) in a solvent that does not adversely influence the reaction at −10° C. to 100° C. for 0.5 to 10 hr, and reacting the obtained compound with compound (VIII) in a solvent that does not adversely influence the reaction, at −10° C. to 100° C. for 0.5 to 30 hr.

This reaction may be carried out in the presence of 1 to mol of a base, per 1 mol of compound (V).

As the base and solvent that does not adversely influence the reaction, those exemplified in the aforementioned Method C can be mentioned.

The amount of compound (VI) to be used is generally 1 to mol, preferably 1 to 5 mol, per 1 mol of compound (V).

The amount of compound (VIII) to be used is generally 1 to 10 mol, preferably 1 to 5 mol, per 1 mol of compound (V).

Compound (VIII) can be produced according to a method known per se.

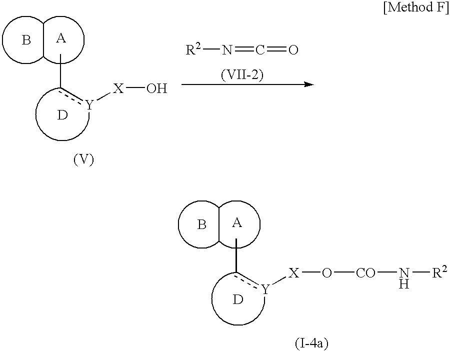

wherein each symbol is as defined above.

In this method, compound (I-4a), which is compound (I-4) wherein R^{1c} is a hydrogen atom, can be produced by reacting compound (V) with compound (VII-2). This reaction is carried out in the same manner as in the reaction described in the aforementioned Method D.

Compound (VII-2) can be produced according to a method known per se.

Compound (I-5), which is compound (I) wherein W is —NR^{1b}CONR^{1a}S(O)ₘR² wherein each symbol is as defined above, is produced, for example, according to the following Method G, Method H1 or Method H2.

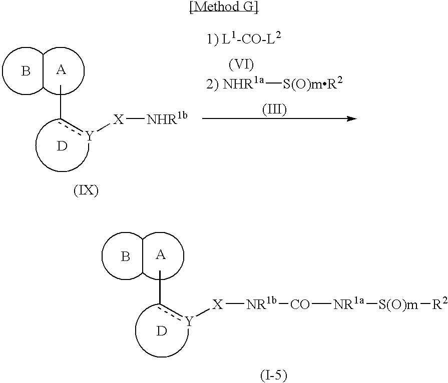

wherein each symbol is as defined above.

In this method, compound (I-5) can be produced by reacting compound (IX) with compound (VI) and (III) successively. This reaction is carried out in the same manner as in the reaction described in the aforementioned Method C.

Compound (IX) can be produced, for example, according to the below-mentioned Method V1 or Method V2 or a method analogous thereto.

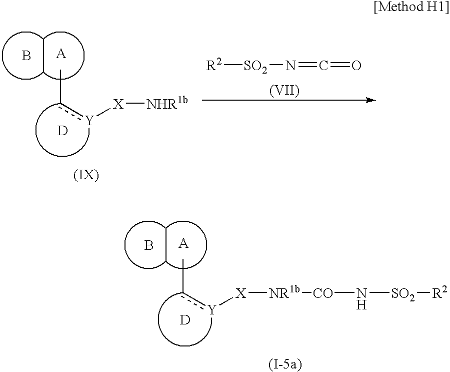

wherein each symbol is as defined above.

In this method, compound (I-5a), which is compound (I-5) wherein R^{1a} is a hydrogen atom and m is 2, can be produced by reacting compound (IX) with compound (VII). This reaction is carried out in the same manner as in the reaction described in the aforementioned Method D.

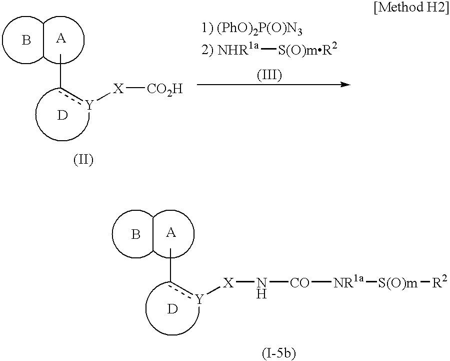

wherein each symbol is as defined above.

In this method, compound (I-5b), which is compound (I-5) wherein R^{1b} is a hydrogen atom, can be produced from compound (II). This reaction is carried out by reacting compound (II) with diphenyl azidophosphate in the presence of a base, in a solvent that does not adversely influence the reaction, at −10° C. to 40° C. for 0.5 to 10 hr, and reacting an isocyanate generated by thermal decomposition of the obtained acylazide with compound (III) in the presence of a base, in a solvent that does not adversely influence the reaction, at 60° C. to 150° C. for 0.5 to 30 hr.

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, pyridine, water and the like can be mentioned. These solvents may be used in a mixture at an appropriate ratio.

The amount of the diphenyl azidophosphate to be used is generally 1 to 10 mol, preferably 1 to 5 mol, per 1 mol of compound (II).

The amount of the base to be used is generally 1 to 10 mol, per 1 mol of compound (II).

The amount of compound (III) to be used is generally 1 to 10 mol, preferably 1 to 5 mol, per 1 mol of compound (II).

Compound (I-6a), which is compound (I) wherein W is —OCONR^{1a}S(O)ₘNR^{1c}R² wherein R^{1a} is a hydrogen atom, m is 2 and the other symbols are as defined above, and compound (I-6b), which is compound (I) wherein W is —OCONR^{1a}S(O)ₘNR^{1c}R² wherein R^{1a} is a C₁₋₆ alkyl group, m is 2 and the other symbols are as defined above, are produced, for example, according to the following Method I.

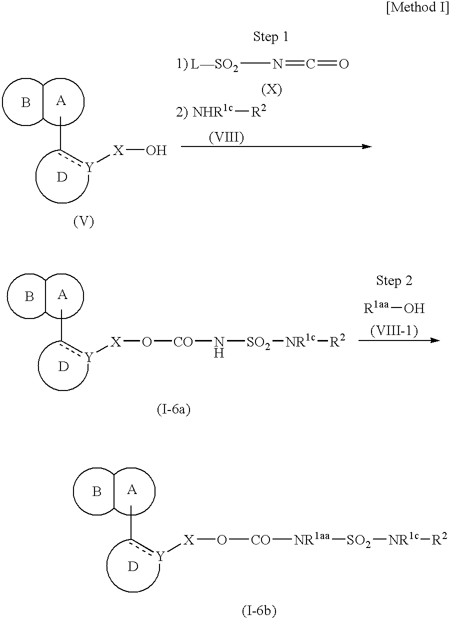

wherein L³ is a leaving group, R^{1aa} is a C₁₋₆ alkyl group, and the other symbols are as defined above.

As the leaving group for L³, those exemplified for the aforementioned L¹ or L² can be mentioned. Of these, it is preferably a halogen atom, particularly preferably a chlorine atom.

### [Step 1]

In this step, compound (I-6a) can be produced from compound (V). This reaction is carried out according to a method known per se, for example, by reacting compound (V) with compound (X) in a solvent that does not adversely influence the reaction, at −10° C. to 100° C. for 0.1 to 10 hr, and reacting the obtained compound with compound (VIII) in a solvent that does not adversely influence the reaction, at −10° C. to 100° C. for 0.5 to 50 hr.

This reaction may be carried out in the presence of 1 to 10 mol of a base, per 1 mol of compound (V).

As specific examples of compound (X), chlorosulfonyl isocyanate and the like can be mentioned.

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, propionitrile, ethyl acetate, pyridine, water and the like can be mentioned. These solvents may be used in a mixture at an appropriate ratio.

The amount of compound (X) to be used is generally 1 to 10 mol, preferably 1 to 5 mol, per 1 mol of compound (V).

The amount of compound (VIII) to be used is generally 1 to 30 mol, preferably 1 to 10 mol, per 1 mol of compound (V).

### [Step 2]

In this step, compound (I-6b) can be produced by reacting compound (I-6a) with compound (VIII-1). This reaction is carried out according to a method known per se, for example, the method described in Synthesis, page 1, (1981) or a method analogous thereto. That is, this reaction is generally carried out in the presence of an organic phosphorus compound and an electrophilic agent, in a solvent that does not adversely influence the reaction.

As the organic phosphorus compound, for example, triphenylphosphine, tributylphosphine and the like can be mentioned.

As the electrophilic agent, for example, diethyl azodicarboxylate, diisopropyl azodicarboxylate, azodicarbonyldipiperizine and the like can be mentioned.

The amount of the organic phosphorus compound and electrophilic agent to be used is generally 1 to 20 mol, per 1 mol of compound (I-6a), respectively.

The amount of compound (VIII-1) to be used is generally 1 to 10 mol, per 1 mol of compound (I-6a).

As the solvent that does not adversely influence the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethylsulfoxide and the like, and the like can be mentioned. These solvents may be used in a mixture at an appropriate ratio.

The reaction temperature is generally −80 to 150° C., preferably −10 to 100° C.

The reaction time is generally 0.5 to 50 hr.

Compound (X) and compound (VIII-1) can be produced according to a method known per se.

Compound (I-7a), which is compound (I) wherein W is —S(O)ₘNR^{1a}COₙR² wherein n is 1 and the other symbols are as defined above, is produced, for example, according to the following Method J.

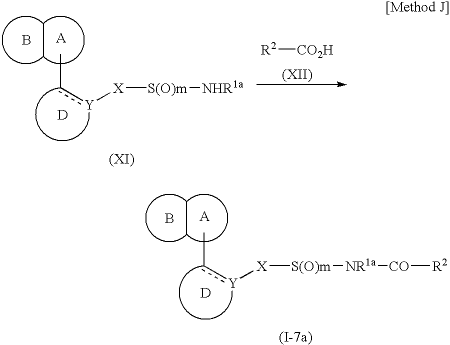

wherein each symbol is as defined above.

In this method, compound (I-7a) can be produced by reacting compound (XI) with compound (XII). This reaction is carried out in the same manner as the condensation reaction in described in the aforementioned Method A1.

Compound (XI) can be produced, for example, according to the below-mentioned Method W or a method analogous thereto. Compound (XII) can be produced according to a method known per se.

Compound (I-7b), which is compound (I) wherein W is —S(O)ₘNR^{1a}COₙR² wherein n is 2 and the other symbols are as defined above, is produced, for example, according to the following Method K or the below-mentioned Method AU.

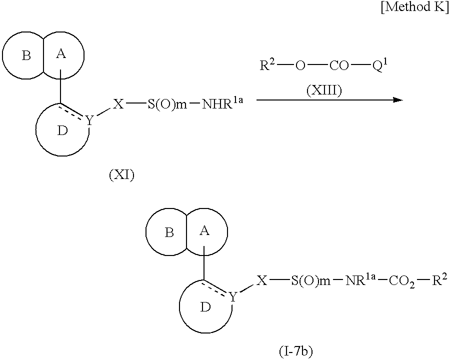

wherein Q¹ is a halogen atom, and the other symbols are as defined above.

The halogen atom for Q¹ is preferably a chlorine atom.

In this method, compound (I-7b) can be produced by reacting compound (XI) with compound (XIII).

The amount of compound (XIII) to be used is generally 0.5 to 200 mol, per 1 mol of compound (XI).

This reaction is carried out in the same manner as in the condensation reaction in described in the aforementioned Method A1.

Compound (XIII) can be produced according to a method known per se.

Compound (I-8), which is compound (I) wherein W is

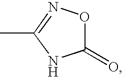

is produced, for example, according to the following Method L.

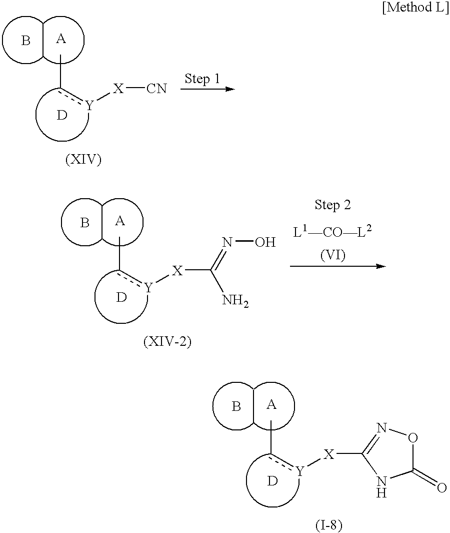

wherein each symbol is as defined above.

### [Step 1]

In this step, compound (XIV-2) can be produced by reacting compound (XIV) with hydroxylamine (or hydroxylammonium chloride). This reaction is carried out in the presence of a base, in a solvent that does not adversely influence the reaction.

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned.

As the solvent that does not adversely influence the reaction, for example, ethers such as diethyl ether, tetrahydrofuran, dioxane and the like; halogenated hydrocarbons such as chloroform, dichloromethane, 1,2-dichloroethane and the like; aromatic hydrocarbons such as benzene, toluene, nitrobenzene and the like; amides such as N,N-dimethylformamide and the like; sulfoxides such as dimethylsulfoxide and the like; ketones such as acetone and the like; ethyl acetate, water and the like can be mentioned. These solvents may be used in a mixture at an appropriate ratio.

The amount of the hydroxylamine to be used is generally 1 to 10 mol, per 1 mol of compound (XIV).

The amount of the base to be used is generally 1 to 10 mol, per 1 mol of compound (XIV).

The reaction temperature is generally −30 to 180° C., preferably −10 to 120° C.

The reaction time is generally 0.5 to 30 hr.

Compound (XIV) can be produced, for example, according to the below-mentioned Method X or a method analogous thereto.

### [Step 2]

In this step, compound (I-8) can be produced by reacting compound (XIV-2) with compound (VI). This reaction is carried out in the presence of a base, in a solvent that does not adversely influence the reaction.

As compound (VI), for example, N,N′-carbonyldiimidazole, diphosgene, triphosgene and the like can be mentioned.

The amount of compound (VI) to be used is generally 1 to 50 mol, preferably 1 to 5 mol, per 1 mol of compound (XIV-2).

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like, and the like can be mentioned.

The amount of the base to be used is generally 1 to 50 mol, preferably 1 to 10 mol, per 1 mol of compound (XIV-2).

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethylsulfoxide and the like; ketones such as acetone and the like; acetonitrile and the like can be mentioned. These solvents may be used in a mixture at an appropriate ratio.

The reaction temperature is generally −80 to 150° C., preferably −10 to 100° C.

The reaction time is generally 0.5 to 30 hr.

Compound (I-9a), which is compound (I) wherein W is a group represented by the formula:

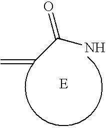

wherein ring E is a 5- or 6-membered heterocycle containing C—CO—NH, which is optionally substituted, and X is —X^{3a}—CH═ wherein X^{3a} is as defined above, and compound (I-9b), which is compound (I) wherein W is a group represented by the formula:

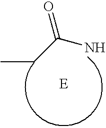

wherein ring E is a 5- or 6-membered heterocycle containing C—CO—NH, which is optionally substituted, and X is —X^{3a}—CH₂— wherein X^{3a} is as defined above, are produced, for example, according to the following Method M.

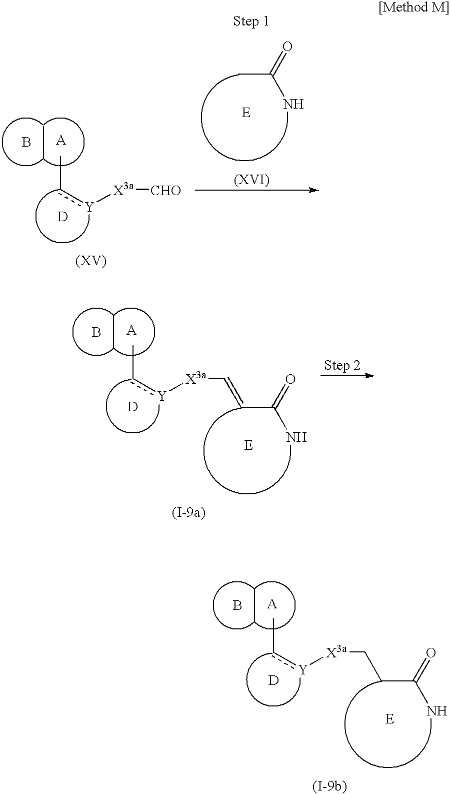

wherein each symbol is as defined above.

As the “5- or 6-membered heterocycle containing C—CO—NH” of the “5- or 6-membered heterocycle containing C—CO—NH, which is optionally substituted” for ring E, rings containing C—CO—NH as a ring-constituting member (e.g., 2,5-dioxopyrroline, 2-oxopyrrolidine, 2,5-dioxopyrrolidine, 2,4-dioxoimidazolidine, 2,6-dioxopiperidine, 2,4-dioxothiazolidine, 1,1-dioxido-3-oxoisothiazolidine, 2,6-dioxohexahydropyrimidine, 1,1-dioxido-3-oxo-1,2-thiazinane), from among rings corresponding to the “5- or 6-membered heterocyclic group containing NH” of the aforementioned “5- or 6-membered heterocyclic group containing NH, which is optionally substituted” for W, can be mentioned. As the substituents of the “5- or 6-membered heterocycle containing C—CO—NH, which is optionally substituted” for ring E, those similar to the substituents of the “5- or 6-membered heterocyclic group containing NH, which is optionally substituted” for W can be mentioned.

### [Step 1]

In this step, compound (I-9a) can be produced by reacting compound (XV) with compound (XVI). This reaction is carried out in the presence of a base, in a solvent that does not adversely influence the reaction.

The amount of compound (XVI) to be used is generally 1 to 10 mol, per 1 mol of compound (XV).

As the base, for example, amines such as piperidine, pyrrolidine, morpholine, pyridine, diethylamine and the like; alkali metal carbonates such as potassium carbonate, sodium carbonate and the like; alkali metal C₁₋₆ alkoxides such as sodium methoxide and the like; alkali metal hydroxides such as potassium hydroxide, sodium hydroxide, lithium hydroxide and the like, and the like can be mentioned.

The amount of the base to be used is generally 0.01 to 10 mol, preferably 0.05 to 5 mol, per 1 mol of compound (XV).

As the solvent that does not adversely influence the reaction, for example, alcohols such as methanol, ethanol, propanol, 2-propanol, 2-methoxyethanol, butanol, isobutanol, tert-butyl alcohol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like; acetic acid and the like can be mentioned. These solvents may be used in a mixture at an appropriate ratio.

The reaction temperature is generally 0 to 150° C., preferably 20 to 120° C.

The reaction time is generally 0.5 to 50 hr.

Compound (XV) can be produced, for example, according to the below-mentioned Method Z1-Method Z3, Step 2 of Method T4, Method AO, Method AQ, Method AV or a method analogous thereto. Compound (XVI) can be produced according to a method known per se.

### [Step 2]

In this step, compound (I-9b) can be produced by subjecting compound (I-9a) to a hydrogenation reaction. This reaction can be carried out, for example, in the presence of a metal catalyst such as palladium-carbon, palladium black, palladium chloride, platinum oxide, palladium black, platinum-palladium, Raney-nickel, Raney-cobalt and the like and a hydrogen source, in a solvent that does not adversely influence the reaction.

The amount of the metal catalyst to be used is generally 0.001 to 1000 mol, preferably 0.01 to 100 mol, per 1 mol of compound (I-9a).

As the hydrogen source, for example, hydrogen gas, formic acid, an amine salt of formic acid, phosphinate, hydrazine and the like can be mentioned.

As the solvent that does not adversely influence the reaction, for example, alcohols such as methanol, ethanol, propanol, 2-propanol, 2-methoxyethanol, butanol, isobutanol, tert-butyl alcohol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as dichloromethane, chloroform, 1,2-dichloroethane, 1,1,2,2-tetrachloroethane and the like; amides such as N,N-dimethylformamide, N,N-dimethylacetamide, N-methylpyrrolidone and the like; ethyl acetate, acetic acid and the like can be mentioned. These solvents may be used in a mixture at an appropriate ratio.

The reaction temperature is generally 0 to 120° C., preferably 10 to 80° C.

The reaction time is generally 0.5 to 200 hr.

Compound (I-9c), which is compound (I) wherein W is

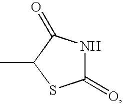

and X is —CH₂CH₂—, is produced, for example, according to the following Method N.

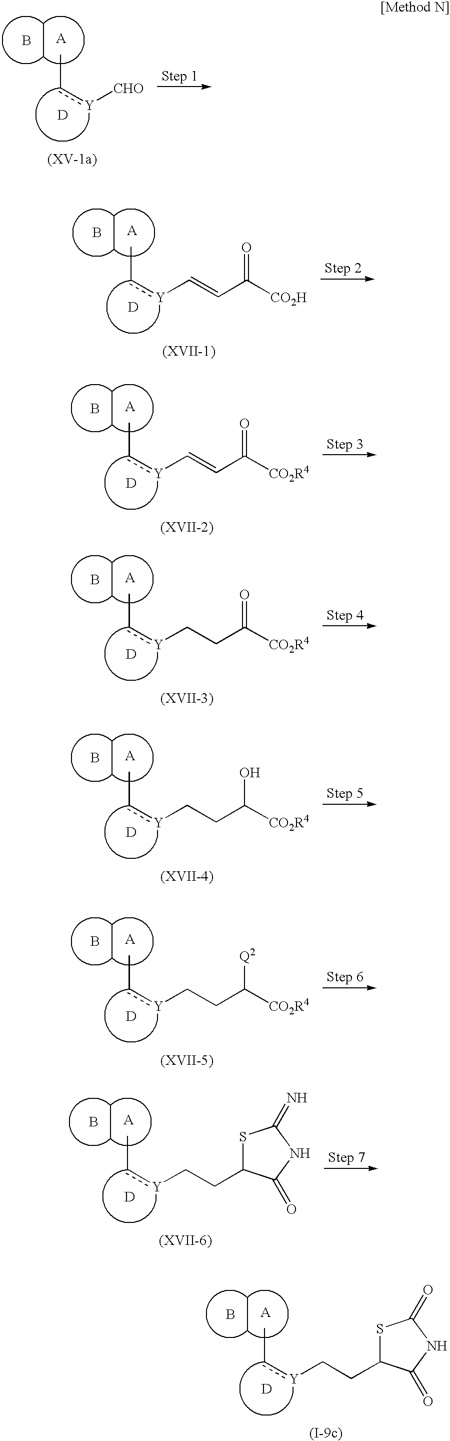

wherein R⁴ is a C₁₋₆ alkyl group, Q² is a halogen atom, and the other symbols are as defined above.

The “C₁₋₆ alkyl group” for R⁴ is preferably methyl, ethyl, tert-butyl or the like.”

The “halogen atom” for Q² is preferably a chlorine atom or a bromine atom.

### [Step 1]

In this step, compound (XVII-1) can be produced by reacting compound (XV-1a) with pyruvic acid. This reaction is carried out in the presence of a base, in a water-containing solvent.

The amount of the pyruvic acid to be used is generally 1 to 10 mol, per 1 mol of compound (XV-1a).

As the base, for example, amines such as piperidine, pyrrolidine, morpholine, pyridine, diethylamine and the like; alkali metal carbonates such as potassium carbonate, sodium carbonate and the like; alkali metal C₁₋₆ alkoxides such as sodium methoxide and the like; alkali metal hydroxides such as potassium hydroxide, sodium hydroxide, lithium hydroxide and the like, and the like can be mentioned.

The amount of the base to be used is generally 0.01 to 10 mol, preferably 0.05 to 5 mol, per 1 mol of compound (XV-1a).

As the water-containing solvent, for example, a mixed solvent of 1 or more solvents selected from alcohols (e.g., methanol, ethanol and the like) and the like and water, and the like can be mentioned.

The reaction temperature is generally 0 to 150° C., preferably 20 to 120° C.

The reaction time is generally 0.5 to 50 hr.

Compound (XV-1a) can be produced, for example, according to the below-mentioned Method Z1, Method Z2, Method AO, Method AQ, Method AV or a method analogous thereto.

### [Step 2]

In this step, compound (XVII-2) can be produced by subjecting compound (XVII-1) to an esterification reaction. This reaction is carried out according to a method known per se, for example, by reacting compound (XVII-1) or a reactive derivative of compound (XVII-1) with an alcohol. As the reactive derivative of compound (XVII-1), for example, acid halides (e.g., acid chlorides, acid bromides), imidazolide, mixed acid anhydrides (e.g., anhydrides with methyl carbonate, ethyl carbonate or isobutyl carbonate, etc.) and the like can be mentioned.

The reaction of compound (XVII-1) with an alcohol is carried out in the presence of an acid.

As the alcohol, methanol, ethanol and the like can be mentioned.

The large excess amount of the alcohol is used as a reaction solvent.

As the acid, mineral acids such as hydrochloric acid, sulfuric acid and the like, and the like can be mentioned.

The amount of the acid to be used is generally 0.05 to 1000 mol, per 1 mol of compound (XVII-1).

The reaction temperature is generally 0 to 200° C., preferably 20 to 120° C.

The reaction time is generally 0.1 to 200 hr.

The method using a reactive derivative of compound (XVII-1) is carried out in the same manner as in the method using a reactive derivative of compound (II) in the aforementioned Method A1 or a method analogous thereto.

### [Step 3]

In this step, compound (XVII-3) can be produced by subjecting compound (XVII-2) to a hydrogenation reaction. This reaction is carried out in the same manner as in the reaction described in Step 2 of the aforementioned Method M.

### [Step 4]

In this method, compound (XVII-4) can be produced by subjecting compound (XVII-3) to a reduction reaction. This reaction is generally carried out in the presence of a reducing agent, in a solvent that does not adversely influence the reaction.

As the reducing agent, for example, metal hydrogen compounds such as sodium bis(2-methoxyethoxy)aluminum hydride, diisobutylaluminum hydride and the like; metal hydrogen complex compounds such as sodium borohydride, sodium cyanoborohydride, lithium aluminum hydride, sodium aluminum hydride and the like, and the like can be mentioned.

The amount of the reducing agent to be used is generally 0.5 to 20 mol, per 1 mol of compound (XVII-3).

As the solvent that does not adversely influence the reaction, for example, alcohols such as methanol, ethanol, propanol, 2-propanol, 2-methoxyethanol, butanol, isobutanol, tert-butyl alcohol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; halogenated hydrocarbons such as dichloromethane, chloroform, 1,2-dichloroethane, 1,1,2,2-tetrachloroethane and the like; water and the like can be mentioned. These solvents may be used in a mixture at an appropriate ratio.

The reaction temperature is generally −30 to 150° C., preferably −10 to 100° C.

The reaction time is generally 0.1 to 100 hr.

### [Step 5]

In this step, compound (XVII-5) can be produced by subjecting compound (XVII-4) to halogenation. This reaction is carried out in the presence of a halogenating agent, in a solvent that does not adversely influence the reaction.

As the halogenating agent, for example, thionyl chloride, oxalyl chloride, phosphoryl chloride, phosphorus trichloride, phosphorus tribromide and the like can be mentioned.

The amount of the halogenating agent to be used is generally 1 to 20 mol, per 1 mol of compound (XVII-4).

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as dichloromethane, chloroform, 1,2-dichloroethane, 1,1,2,2-tetrachloroethane and the like; amides such as N,N-dimethylformamide, N,N-dimethylacetamide and the like, and the like can be mentioned. These solvents may be used in a mixture at an appropriate ratio.

The reaction temperature is generally −30 to 150° C., preferably −10 to 100° C.

The reaction time is generally 0.1 to 50 hr.

### [Step 6]

In this step, compound (XVII-6) can be produced by reacting compound (XVII-5) with thiourea. This reaction is carried out in the presence of sodium acetate or potassium acetate, in a solvent that does not adversely influence the reaction. In addition, the reaction efficiency can be improved by adding 1 to 1.5 mol of sodium iodide or potassium iodide, per 1 mol of compound (XVII-5).

The amount of the thiourea to be used is generally 1 to 10 mol, per 1 mol of compound (XVII-5).

The amount of the sodium acetate or potassium acetate to be used is generally 1 to 10 mol, per 1 mol of compound (XVII-5).

As the solvent that does not adversely influence the reaction, for example, alcohols such as methanol, ethanol, propanol, 2-propanol, 2-methoxyethanol, butanol, isobutanol, tert-butyl alcohol and the like; amides such as N,N-dimethylformamide, N,N-dimethylacetamide, N-methylpyrrolidone and the like; sulfoxides such as dimethyl sulfoxide, sulforan and the like, and the like can be mentioned. These solvents may be used in a mixture at an appropriate ratio.

The reaction temperature is generally 0 to 180° C., preferably 50 to 150° C.

The reaction time is generally 0.5 to 100 hr.

### [Step 7]

In this step, compound (I-9c) can be produced by subjecting compound (XVII-6) to hydrolysis. This reaction is carried out in the presence of an acid, in a solvent that does not adversely influence the reaction.

As the acid, mineral acids such as hydrochloric acid, sulfuric acid and the like, and the like can be mentioned.

The amount of the acid to be used is generally 0.01 to 1000 mol, per 1 mol of compound (XVII-6).

As the solvent that does not adversely influence the reaction, for example, alcohols such as methanol, ethanol, propanol, 2-propanol, 2-methoxyethanol, butanol, isobutanol, tert-butyl alcohol and the like; amides such as N,N-dimethylformamide, N,N-dimethylacetamide, N-methylpyrrolidone and the like; sulfoxides such as dimethyl sulfoxide, sulforan and the like, and the like can be mentioned. These solvents may be used in a mixture at an appropriate ratio.

The reaction temperature is generally 20 to 150° C., preferably 50 to 120° C.

The reaction time is generally 0.5 to 50 hr.

Compound (I-10), which is compound (I) wherein W is —ONR^{1a}CONR^{1c}R² wherein each symbol is as defined above, is produced, for example, according to the following Method O1 or Method O2.

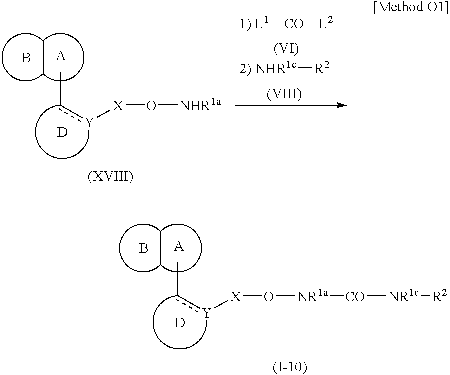

wherein each symbol is as defined above.

In this method, compound (I-10) can be produced by reacting compound (XVIII) with compound (VI) and (VIII) successively. This reaction is carried out in the same manner as in the reaction described in the aforementioned Method E.

Compound (XVIII) can be produced, for example, according to the below-mentioned Method Y or a method analogous thereto.

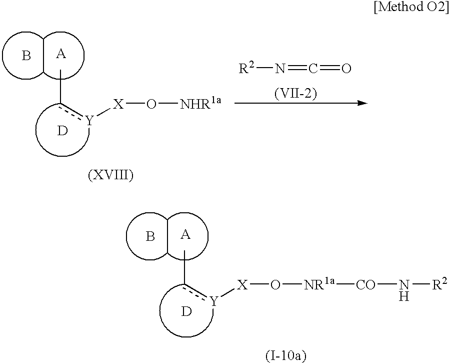

wherein each symbol is as defined above.

In this method, compound (I-10a), which is compound (I-10) wherein R^{1c} is a hydrogen atom, can be produced by reacting compound (XVIII) with compound (VII-2). This reaction is carried out in the same manner as in the reaction described in the aforementioned Method D.

Compound (I-11a), which is compound (I) wherein W is —ONR^{1a}COₙR² wherein n is 1 and the other symbols are as defined above, is produced, for example, according to the following Method P.

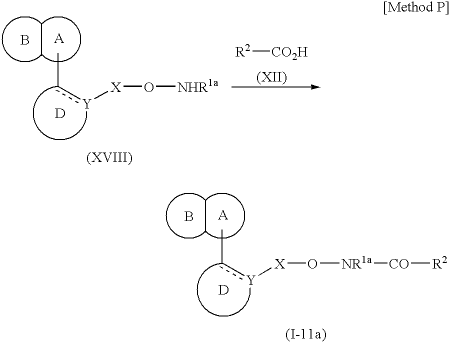

wherein each symbol is as defined above.

In this method, compound (I-11a) can be produced by reacting compound (XVIII) with compound (XII). This reaction is carried out in the same manner as in the aforementioned Method A1.

Compound (I-11b), which is compound (I) wherein W is —ONR^{1a}COₙR² wherein n is 2 and the other symbols are as defined above, is produced, for example, according to the following Method Q.

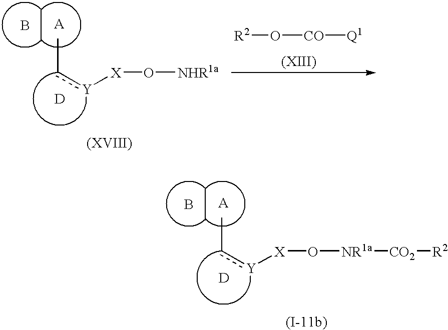

wherein each symbol is as defined above.

In this method, compound (I-11b) can be produced by reacting compound (XVIII) with compound (XIII). This reaction is carried out in the same manner as in the aforementioned Method A1.

Compound (I-12), which is compound (I) wherein W is —CONR^{1a}CONR^{1c}R² wherein each symbol is as defined above, is produced, for example, according to the following Method R.

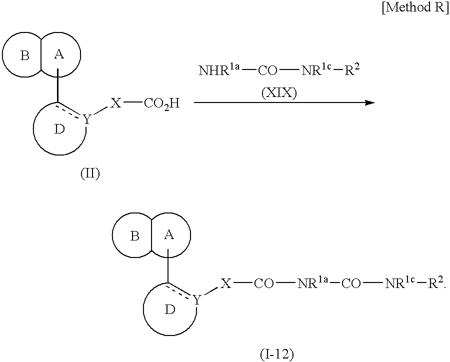

wherein each symbol is as defined above.

In this method, compound (I-12) can be produced by reacting compound (II) with compound (XIX). This reaction is carried out in the same manner as in the aforementioned Method A1.

Compound (XIX) can be produced according to a method known per se.

Compound (I-13), which is compound (I) wherein W is a group represented by

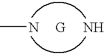

wherein ring G is a 5- or 6-membered heterocycle containing NH and further containing, besides the NH, at least one nitrogen atom, which is optionally substituted, is produced, for example, according to the following Method S1.

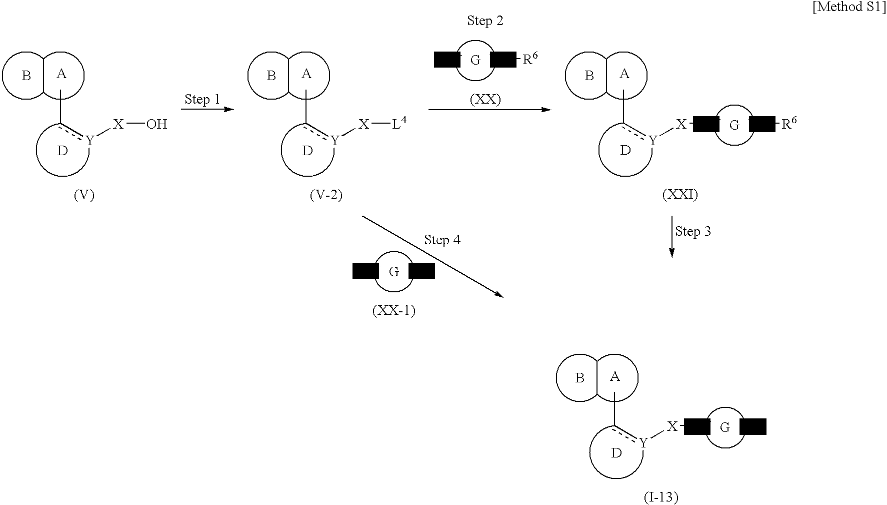

wherein L⁴ is a leaving group, R⁶ is a nitrogen atom-protecting group, and the other symbols are as defined above.

As the “5- or 6-membered heterocycle containing NH and further containing, besides the NH, at least one nitrogen atom” of the “5- or 6-membered heterocycle containing NH and further containing, besides the NH, at least one nitrogen atom, which is optionally substituted” for ring G, rings further containing, as a ring-constituting member besides “NH”, at least one nitrogen atom (e.g., imidazolidine, 2-oxoimidazolidine, 2,4-dioxoimidazolidine, tetrahydropyrimidine, 2,6-dioxohexahydropyrimidine, 1,1-dioxido-3-oxothiadiazolidine, 2-oxopiperazine), from among rings corresponding to the “5- or 6-membered heterocyclic group containing NH” of the “5- or 6-membered heterocyclic group containing NH, which is optionally substituted” for W, can be mentioned. As the substituents for the ring G, those similar to the substituents of the “5- or 6-membered heterocyclic group containing NH, which is optionally substituted” for W, can be mentioned.

As the “leaving group” for L⁴, a halogen atom, —OSO₂R³ wherein R³ is as defined above, and the like can be mentioned.

As the “nitrogen atom-protecting group” for R⁶, a C₁₋₆ alkoxy-carbonyl (e.g., tert-butoxycarbonyl), a C₇₋₁₃ aralkyloxy-carbonyl (e.g., benzyloxycarbonyl), tert-butyl, benzyl, a substituted benzyl (e.g., 4-methoxybenzyl, 2,4-dimethoxybenzyl) and the like can be mentioned.

### [Step 1]

In this step, compound (V-2) can be produced by subjecting compound (V) to sulfonylation or halogenation.

The sulfonylation of compound (V) is carried out using a sulfonyl halide in the presence of a base, in a solvent that does not adversely influence the reaction.

The sulfonyl halide is preferably methanesulfonyl chloride, p-toluenesulfonyl chloride or the like.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethylsulfoxide and the like; acetonitrile and the like can be mentioned. These solvents may be used in a mixture at an appropriate ratio.

The amount of the sulfonyl halide to be used is generally 1 to 10 mol, preferably 1 to 5 mol, per 1 mol of compound (V).

The amount of the base to be used is generally 1 to 10 mol, preferably 1 to 5 mol, per 1 mol of compound (V).

The reaction temperature is generally −30 to 150° C., preferably −10 to 100° C.

The reaction time is generally 0.1 to 50 hr.

The halogenation of compound (V) is carried out in the same manner as in the reaction described in the aforementioned Step 5 of Method N.

### [Step 2]

In this step, compound (XXI) can be produced by reacting compound (V-2) with compound (XX). This reaction is generally carried out in the presence of a base, in a solvent that does not adversely influence the reaction.

As the base, for example, alkali metal salts such as potassium hydroxide, sodium hydroxide, sodium hydrogencarbonate, sodium carbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like, and the like can be mentioned. These solvents may be used in a mixture at an appropriate ratio.

The amount of compound (XX) to be used is generally 1 to 20 mol, preferably 1 to 10 mol, per 1 mol of compound (V-2).

The amount of the base to be used is generally 1 to 20 mol, preferably 1 to 10 mol, per 1 mol of compound (V-2).

The reaction temperature is generally −30 to 180° C., preferably −10 to 120° C.

The reaction time is generally 0.5 to 100 hr.

Compound (XX) can be produced according to a method known per se.

### [Step 3]

In this step, compound (I-13) can be produced by subjecting compound (XXI) to deprotection.

When R⁶ is tert-butoxycarbonyl, tert-butyl, 4-methoxybenzyl or 2,4-dimethoxybenzyl, the reaction is carried out in the presence of an acid, in a solvent that does not adversely influence.

As the acid, for example, mineral acids such as hydrochloric acid, sulfuric acid and the like; organic acids such as trifluoroacetic acid, p-toluenesulfonic acid and the like; solutions prepared by dissolving hydrogen chloride in methanol, ethyl acetate and the like, such as hydrogen chloride-methanol solution, hydrogen chloride-ethyl acetate solution and the like can be mentioned.

As the solvent that does not adversely influence the reaction, ethers such as diethyl ether, diisopropyl ether, tert-butyl methyl ether, tetrahydrofuran, dioxane, 1,2-dimethoxyethane and the like; alcohols such as methanol, ethanol, isopropanol, tert-butyl alcohol and the like; ethyl acetate, water and the like can be mentioned. These solvents may be used in a mixture at an appropriate ratio.

The amount of the acid to be used is generally 0.01 to 1000 mol, preferably 0.1 to 100 mol, per 1 mol of compound (XXI).

The reaction temperature is generally −80 to 150° C., preferably −10 to 100° C.

The reaction time is generally 0.1 to 30 hr.

When R⁶ is benzyloxycarbonyl or benzyl, for example, the reaction can be carried out in the presence of a metal catalyst such as palladium-carbon, palladium black, palladium chloride, platinum oxide, palladium black, platinum-palladium, Raney-nickel, Raney-cobalt and the like and a hydrogen source, in a solvent that does not adversely influence.

The amount of the metal catalyst to be used is generally 0.001 to 1000 mol, preferably 0.01 to 100 mol, per 1 mol of compound (XXI).

As the hydrogen source, for example, hydrogen gas, formic acid, an amine salt of formic acid, phosphinate, hydrazine and the like can be mentioned.

As the solvent that does not adversely influence the reaction, for example, alcohols such as methanol, ethanol, propanol, 2-propanol, 2-methoxyethanol, butanol, isobutanol, tert-butyl alcohol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as dichloromethane, chloroform, 1,2-dichloroethane, 1,1,2,2-tetrachloroethane and the like; amides such as N,N-dimethylformamide, N,N-dimethylacetamide, N-methylpyrrolidone and the like; ethyl acetate, acetic acid and the like can be mentioned. These solvents may be used in a mixture at an appropriate ratio.

The reaction temperature is generally 0 to 120° C., preferably 10 to 80° C.

The reaction time is generally 0.5 to 100 hr.

### [Step 4]

In this step, compound (I-13) can be produced by reacting compound (V-2) with compound (XX-1). This reaction is carried out in the same manner as in the reaction described in the aforementioned Step 2 of this method.

Compound (XX-1) can be produced according to a method known per se.

Compound (I-14b), which is compound (I) wherein X is —X²—CH₂CH₂— wherein X² is a bond or a straight chain C₁₋₂ alkylene, is produced, for example, according to the following Method S2.

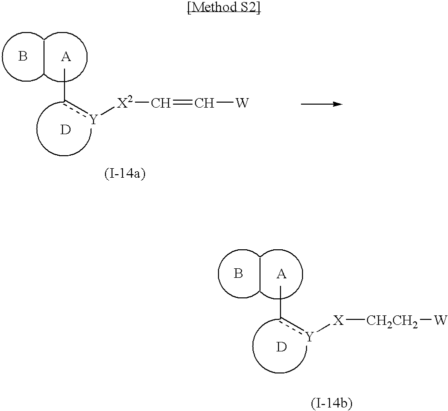

wherein each symbol is as defined above.

In this method, compound (I-14b) can be produced by subjecting compound (I-14a) to a hydrogenation reaction. This reaction is carried out in the same manner as in the reaction described in Step 2 of the aforementioned Method M.

Compound (I-14a) can be produced, for example, according to the aforementioned Method A1, Method B, Method J, Method K, Method L, Method R, the below-mentioned Method AA to Method AC, Method AF to Method AL or Method AU, or a method analogous thereto.

Compound (II) used in the aforementioned Method A1, Method B, Method H2 and Method R as a starting material compound is produced, for example, according to the following Method T1-Method T5.

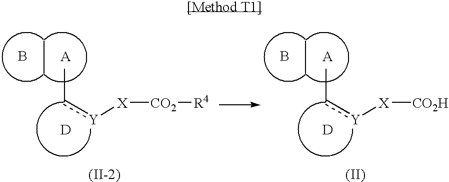

wherein each symbol is as defined above.

In this method, compound (II) can be produced by subjecting compound (II-2) to hydrolysis. This reaction is carried out in the presence of an acid or a base, in a water-containing solvent, according to a method known per se.

As the acid, for example, mineral acids such as hydrochloric acid, sulfuric acid, hydrobromic acid and the like; solutions prepared by dissolving hydrogen chloride in methanol, ethyl acetate and the like, such as hydrogen chloride-methanol solution, hydrogen chloride-ethyl acetate solution and the like; organic acids such as trifluoroacetic acid, p-toluenesulfonic acid, acetic acid and the like, and the like can be mentioned.

As the base, for example, alkali metal carbonates such as potassium carbonate, sodium carbonate and the like; alkali metal C₁₋₆ alkoxides such as sodium methoxide and the like; alkali metal hydroxides such as potassium hydroxide, sodium hydroxide, lithium hydroxide and the like, and the like can be mentioned.

The amount of the acid or base to be used is generally an excess amount, per 1 mol of compound (II-2). The amount of the acid to be used is preferably 2 to 100 mol, per 1 mol of compound (II-2). The amount of the base to be used is 1 to 10 mol, per 1 mol of compound (II-2).

As the water-containing solvent, for example, a mixed solvent 1 or more solvents selected from alcohols such as methanol, ethanol and the like; ethers such as tetrahydrofuran, dioxane, diethyl ether and the like; dimethyl sulfoxide, acetone and the like, and water, and the like can be mentioned.

The reaction temperature is generally −30 to 150° C., preferably −10 to 100° C.

The reaction time is generally 0.1 to 50 hr.

Compound (II-2) can be produced, for example, according to Step 3 to Step 5 of the aforementioned Method N, Step 1 or Step 2 of the below-mentioned Method T2, Method AM, Method AN, Method AP or a method analogous thereto.

Compound (II-1a), which is compound (II) wherein X is —X²—CH═CR⁵— wherein R⁵ is as defined below, and X² is as defined above, and compound (II-1b), which is compound (II) wherein X is —X²—CH₂CHR⁵— wherein R⁵ is as defined below, and X² is as defined above, are produced, for example, according to the following Method T2.

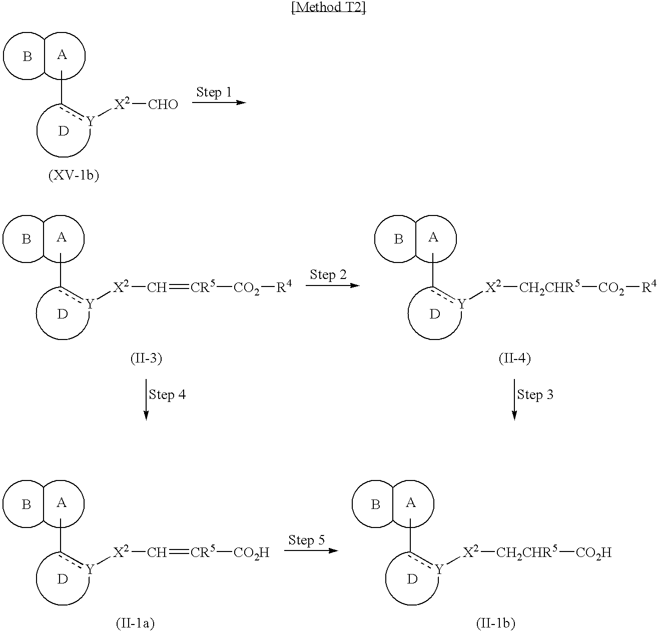

wherein R⁵ is a C₁₋₃ alkyl group, and the other symbols are as defined above.

### [Step 1]

In this step, compound (II-3) can be produced by subjecting compound (XV-1b) to a carbon addition reaction. This reaction is generally carried out using an organic phosphorus reagent, in the presence of a base, in a solvent that does not adversely influence the reaction.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the organic phosphorus reagent, for example, ethyl (diethoxyphosphoryl)acetate, ethyl 2-(diethoxyphosphoryl)propanoate, tert-butyl (diethoxyphosphoryl)acetate and the like can be mentioned.

As the solvent that does not adversely influence the reaction, for example, aromatic hydrocarbons such as benzene, toluene, xylene and the like; aliphatic hydrocarbons such as hexane, heptane and the like; alcohols such as methanol, ethanol and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethylsulfoxide and the like, and the like can be mentioned. These solvents may be used in a mixture at an appropriate ratio.

The amount of the base to be used is generally 1 to 20 mol, preferably 1 to 5 mol, per 1 mol of compound (XV-1b).

The amount of the organic phosphorus reagent to be used is generally 1 to 20 mol, preferably 1 to 5 mol, per 1 mol of compound (XV-1b).

The reaction temperature is generally −80 to 150° C., preferably −10 to 100° C.

The reaction time is generally 0.1 to 30 hr.

Compound (XV-1b) can be produced, for example, according to the below-mentioned Step 2 of Method T4, Method Z1 to Method Z3, Method AO, Method AQ, Method AV or a method analogous thereto.

### [Step 2]

In this step, compound (II-4) can be produced by subjecting compound (II-3) to a hydrogenation reaction. This reaction is carried out in the same manner as in the reaction described in Step 2 of the aforementioned Method M.

### [Step 3]

In this step, compound (II-1b) can be produced by subjecting compound (II-4) to hydrolysis. This reaction is carried out in the same manner as in the reaction described in the aforementioned Method T1.

### [Step 4]

In this step, compound (II-1a) can be produced by subjecting compound (II-3) to hydrolysis. This reaction is carried out in the same manner as in the reaction described in the aforementioned Method T1.

### [Step 5]

In this step, compound (II-1b) can be produced by subjecting compound (II-1a) to a hydrogenation reaction. This reaction is carried out in the same manner as in the reaction described in Step 2 of the aforementioned Method M.

Compound (II-1c), which is compound (II) wherein X is —CH═CR^{5a}— wherein R^{5a} is a hydrogen atom or a C₁₋₃ alkyl group, is produced, for example, according to the following Method T3.

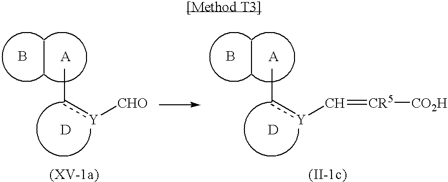

wherein each symbol is as defined above.

In this method, compound (II-1c) can be produced by subjecting compound (XV-1a) to carbon addition reaction. This reaction is generally carried out using malonic acid or a substituted malonic acid, in the presence of a base, in a solvent that does not adversely influence the reaction.

As the substituted malonic acid, methyl malonate, ethyl malonate, propyl malonate and the like can be mentioned.

The amount of the malonic acid or substituted malonic acid to be used is generally 1 to 50 mol, preferably 1 to 20 mol, per 1 mol of compound (XV-1a).

As the base, for example, amines such as piperidine, pyrrolidine, morpholine, pyridine, diethylamine and the like; alkali metal carbonates such as potassium carbonate, sodium carbonate and the like; alkali metal C₁₋₆ alkoxides such as sodium methoxide and the like; alkali metal hydroxides such as potassium hydroxide, sodium hydroxide, lithium hydroxide and the like, and the like can be mentioned.

The amount of the base to be used is generally 0.1 to 50 mol, preferably 1 to 20 mol, per 1 mol of compound (XV-1a).

As the solvent that does not adversely influence the reaction, for example, alcohols such as methanol, ethanol, propanol, 2-propanol, 2-methoxyethanol, butanol, isobutanol, tert-butyl alcohol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like; acetic acid, pyridine and the like can be mentioned. These solvents may be used in a mixture at an appropriate ratio.

The reaction temperature is generally 0 to 200° C., preferably 20 to 150° C.

The reaction time is generally 0.5 to 100 hr.

Compound (II-1d), which is compound (II) wherein X is methylene, is produced, for example, according to the following Method T4.

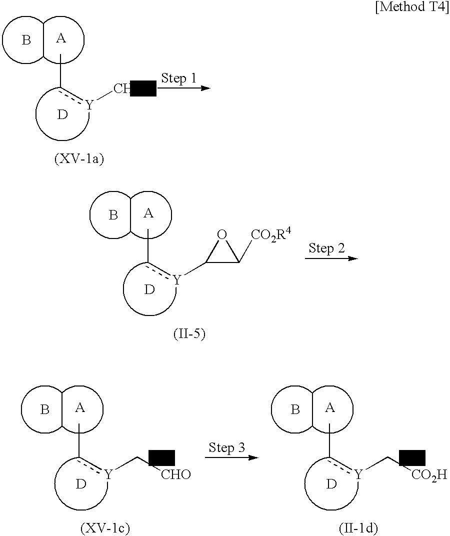

wherein each symbol is as defined above.

### [Step 1]

In this step, compound (II-5) can be produced by reacting compound (XV-1a) with a haloacetate. This reaction is generally carried out in the presence of a base, in a solvent that does not adversely influence.

As the haloacetate, ethyl bromoacetate, ethyl chloroacetate and the like can be mentioned.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like; alcohols such as methanol, ethanol, isopropanol, tert-butyl alcohol and the like, and the like can be mentioned. These solvents may be used in a mixture at an appropriate ratio.

The amount of the haloacetate to be used is generally 1 to 50 mol, preferably 1 to 10 mol, per 1 mol of compound (XV-1a).

The amount of the base to be used is generally 1 to 30 mol, preferably 1 to 10 mol, per 1 mol of compound (XV-1a).

The reaction temperature is generally −80 to 150° C., preferably −20 to 100° C.

The reaction time is generally 0.5 to 20 hr.

### [Step 2]

In this step, compound (XV-1c) can be produced by subjecting compound (II-5) to hydrolysis, and subjecting the obtained carboxylic acid to a decarboxylation reaction in the presence of an acid.

The hydrolysis of compound (II-5) is carried out in the same manner as in the reaction described in the aforementioned Method T1.

The decarboxylation reaction of the carboxylic acid obtained by the hydrolysis of compound (II-5) is carried out in the presence of an acid, in a solvent that does not adversely influence.

As the solvent that does not adversely influence the reaction, those similar to the water-containing solvent used for the hydrolysis of the aforementioned Method T1, can be mentioned.

As the acid, mineral acids such as hydrochloric acid, sulfuric acid and the like; organic acids such as acetic acid and the like, and the like can be mentioned.

The amount of the acid to be used is generally 0.01 to 1000 mol, per 1 mol of compound (II-5).

The reaction temperature is generally −30 to 150° C., preferably −10 to 100° C.

The reaction time is generally 0.5 to 30 hr.

### [Step 3]

In this step, compound (II-1d) can be produced by subjecting compound (XV-1c) to an oxidization reaction. This reaction is carried out according to a method known per se, for example, using sodium dihydrogenphosphate, sodium chlorite and 2-methyl-2-butene, in a solvent that does not adversely influence the reaction.

As the solvent that does not adversely influence the reaction, for example, a mixed solvent of tert-butyl alcohol and water; a mixed solvent of tert-butyl alcohol, tetrahydrofuran and water, and the like can be mentioned.

The amount of the sodium dihydrogenphosphate, sodium chlorite and 2-methyl-2-butene to be used is generally 1 to 50 mol, preferably 1 to 20 mol, per 1 mol of compound (XV-1c), respectively.

The reaction temperature is generally −30 to 150° C., preferably −10 to 80° C.

The reaction time is generally 0.5 to 30 hr.

Compound (II-1e), which is compound (II) wherein X is —X^{1a}—O—CH₂— wherein X^{1a} is as defined above, is produced, for example, according to the following Method T5.

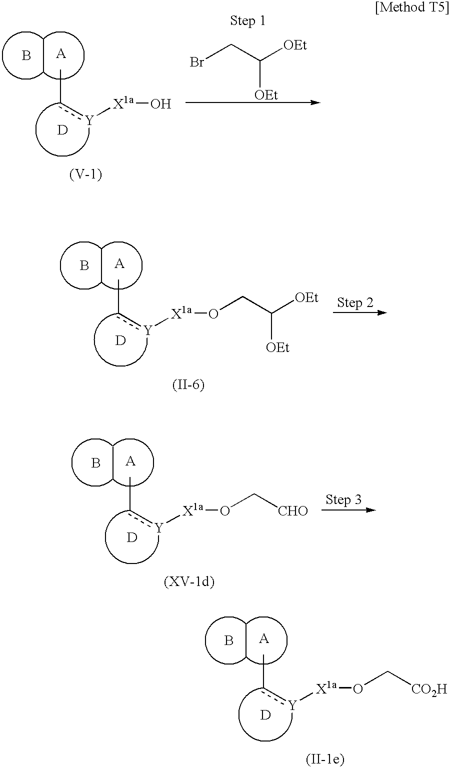

wherein each symbol is as defined above.

### [Step 1]

In this step, compound (II-6) can be produced by reacting compound (V-1) with 2-bromo-1,1-diethoxyethane. This reaction is generally carried out in the presence of a base, in a solvent that does not adversely influence.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like, and the like can be mentioned. These solvents may be used in a mixture at an appropriate ratio.

The amount of the 2-bromo-1,1-diethoxyethane to be used is generally 1 to 20 mol, preferably 1 to 10 mol, per 1 mol of compound (V-1).

The amount of the base to be used is generally 1 to 20 mol, preferably 1 to 10 mol, per 1 mol of compound (V-1).

The reaction temperature is generally −30 to 150° C., preferably −10 to 100° C.

The reaction time is generally 0.5 to 100 hr.

Compound (V-1) can be produced, for example, according to the below-mentioned Method U1 or Method U2 or a method analogous thereto.

### [Step 2]

In this step, compound (XV-1d) can be produced by subjecting compound (II-6) to a deacetalation reaction. This reaction is carried out in the presence of an acid, in a solvent that does not adversely influence, according to a method known per se.

As the acid, for example, mineral acids such as hydrochloric acid, sulfuric acid and the like; organic acids such as trifluoroacetic acid, p-toluenesulfonic acid and the like; solutions prepared by dissolving hydrogen chloride in methanol, ethyl acetate and the like, such as hydrogen chloride-methanol solution, hydrogen chloride-ethyl acetate solution and the like can be mentioned.

As the solvent that does not adversely influence the reaction, ethers such as diethyl ether, diisopropyl ether, tert-butyl methyl ether, tetrahydrofuran, dioxane, 1,2-dimethoxyethane and the like; alcohols such as methanol, ethanol, isopropanol, tert-butyl alcohol and the like; ethyl acetate, water and the like can be mentioned. These solvents may be used in a mixture at an appropriate ratio.

The amount of the acid to be used is generally 0.01 to 1000 mol, per 1 mol of compound (II-6).

The reaction temperature is generally −30 to 150° C., preferably −10 to 100° C.

The reaction time is generally 0.1 to 20 hr.

### [Step 3]

In this step, compound (II-1e) can be produced by subjecting compound (XV-1d) to an oxidization reaction. This reaction is carried out in the same manner as in the reaction described in Step 3 of the aforementioned Method T4.

Compound (V) used as a starting material compound in the aforementioned Method C, Method D, Method E, Method F, Method I and Method S1, and the below-mentioned Method Y, compound (V-1) used as a starting material compound in the aforementioned Method T5, and compound (V-1a) used as a starting material compound in the below-mentioned Method Z3, are produced, for example, according to the following Method U1 or Method U2.

Compound (V-1a), which is compound (V) wherein X is —X^{3a}—CH₂— wherein X^{3a} is as defined above, is produced, for example, according to the following Method U1 or Method U2.

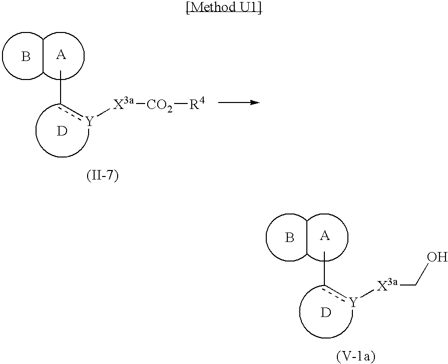

wherein each symbol is as defined above.

In this method, compound (V-1a) can be produced by subjecting compound (II-7) to a reduction reaction. This reaction is carried out in the same manner as in the reaction described in Step 4 of the aforementioned Method N.

Compound (II-7) can be produced, for example, according to Step 1 or Step 2 of the aforementioned Method T2, the below-mentioned Method AM, Method AN, Method AP or a method analogous thereto.

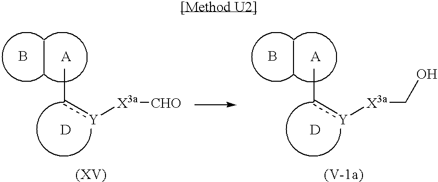

wherein each symbol is as defined above.

In this method, compound (V-1a) can be produced by subjecting compound (XV) to a reduction reaction. This reaction is carried out in the same manner as in the reaction described in Step 4 of the aforementioned Method N.

Compound (IX) used as a starting material compound in the aforementioned Method G and Method H1 is produced, for example, according to the following Method V1 or Method V2.

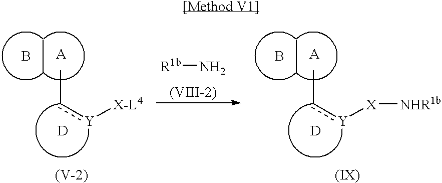

wherein each symbol is as defined above.

In this method, compound (IX) can be produced by reacting compound (V-2) with compound (VIII-2). This reaction is carried out in the same manner as in the reaction described in Step 2 of the aforementioned Method S1.

Compound (VIII-2) can be produced according to a method known per se.

Compound (IX-1a), which is compound (IX) wherein R^{1b} is a hydrogen atom, is produced, for example, according to the following Method V2.

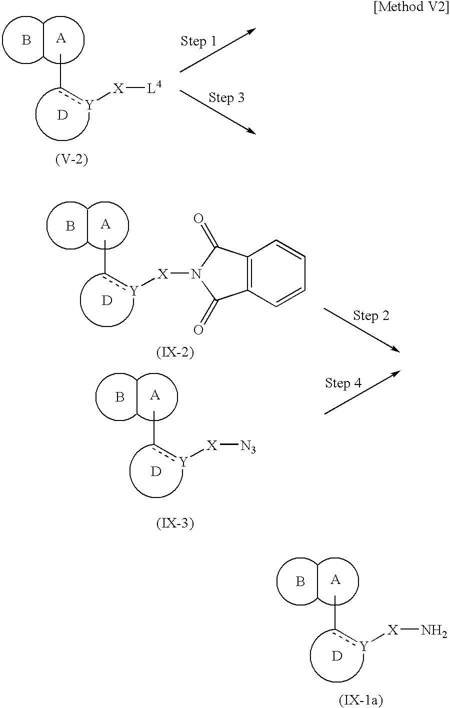

wherein each symbol is as defined above.

### [Step 1]

In this step, compound (IX-2) can be produced by reacting compound (V-2) with potassium phthalimide. This reaction is carried out in a solvent that does not adversely influence the reaction.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethylsulfoxide and the like, and the like can be mentioned. These solvents may be used in a mixture at an appropriate ratio.

The amount of the potassium phthalimide to be used is generally 1 to 10 mol, preferably 1 to 5 mol, per 1 mol of compound (V-2).

The reaction temperature is generally −30 to 150° C., preferably −10 to 100° C.

The reaction time is generally 0.5 to 50 hr.

### [Step 2]

In this step, compound (IX-1a) can be produced by subjecting compound (IX-2) to hydrolysis using an acid or a base. This reaction is carried out in a solvent that does not adversely influence the reaction.

As the acid, for example, mineral acids such as sulfuric acid and the like can be mentioned. As the base, for example, hydrazine hydrate can be mentioned. Of these, hydrazine hydrate is preferable.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; alcohols such as methanol, ethanol, isopropanol, tert-butyl alcohol and the like; water and the like can be mentioned. These solvents may be used in a mixture at an appropriate ratio.

The amount of the acid or base to be used is generally 1 to 100 mol, per 1 mol of compound (IX-2).

The reaction temperature is generally −10 to 150° C., preferably 10 to 100° C.

The reaction time is generally 0.5 to 50 hr.

### [Step 3]

In this step, compound (IX-3) can be produced by reacting compound (V-2) with an azide compound. This reaction is carried out in a solvent that does not adversely influence the reaction.

As the azide compound, sodium azide and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethylsulfoxide and the like, and the like can be mentioned. These solvents may be used in a mixture at an appropriate ratio.

The amount of the azide compound to be used is generally 1 to 10 mol, preferably 1 to 5 mol, per 1 mol of compound (V-2).

The reaction temperature is generally −10 to 150° C., preferably 0 to 100° C.

The reaction time is generally 0.1 to 30 hr.

### [Step 4]

In this step, compound (IX-1a) can be produced by subjecting compound (IX-3) to a reduction reaction. This reaction is carried out in the same manner as in the reaction described in Step 2 of the aforementioned Method M or Step 4 of the aforementioned Method N.

Compound (XI-1a), which is compound (XI) (used as a starting material compound in the aforementioned Method J and Method K) wherein R^{1a} is a hydrogen atom, m is 2, and X is —CH═CH—, and compound (XI-1b), which is compound (XI) wherein R^{1a} is a hydrogen atom, m is 2, and X is —CH₂CH₂—, are produced, for example, according to the following Method W.

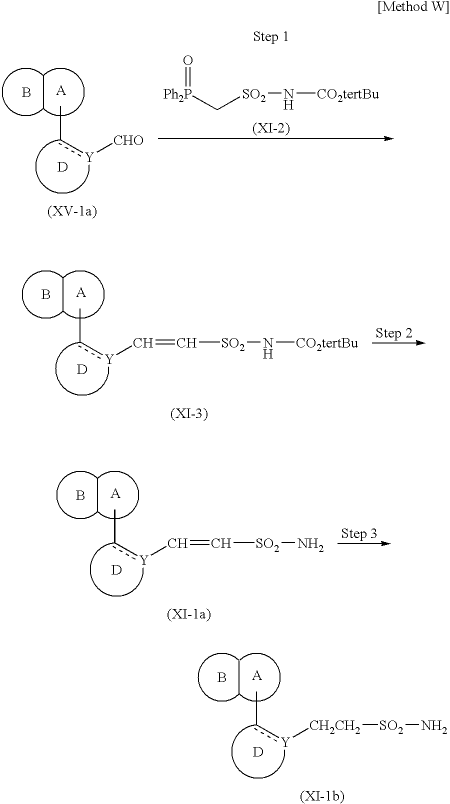

wherein each symbol is as defined above.

### [Step 1]

In this step, compound (XI-3) can be produced by reacting compound (XV-1a) with compound (XI-2). This reaction is carried out according to a method known per se (e.g., the method described in Synthesis, page 2321 (2003), Step 1 of the aforementioned Method T2 or a method analogous thereto etc.).

Compound (XI-2) can be produced according to a method known per se.

### [Step 2]

In this step, compound (XI-1a) can be produced by subjecting compound (XI-3) to deprotection. This reaction is carried out in the presence of an acid, in a solvent that does not adversely influence, according to a method known per se.

As the acid, for example, mineral acids such as hydrochloric acid, sulfuric acid and the like; organic acids such as trifluoroacetic acid, p-toluenesulfonic acid and the like; solutions prepared by dissolving hydrogen chloride in methanol, ethyl acetate and the like, such as hydrogen chloride-methanol solution, hydrogen chloride-ethyl acetate solution and the like can be mentioned.

As the solvent that does not adversely influence the reaction, ethers such as diethyl ether, diisopropyl ether, tert-butyl methyl ether, tetrahydrofuran, dioxane, 1,2-dimethoxyethane and the like; alcohols such as methanol, ethanol, isopropanol, tert-butyl alcohol and the like; ethyl acetate, water and the like can be mentioned. These solvents may be used in a mixture at an appropriate ratio.

The amount of the acid to be used is generally 0.01 to 1000 mol, per 1 mol of compound (XI-3).

The reaction temperature is generally −80 to 150° C., preferably −10 to 100° C.

The reaction time is generally 0.1 to 30 hr.

### [Step 3]

In this step, compound (XI-1b) can be produced by subjecting compound (XI-1a) to a hydrogenation reaction. This reaction is carried out in the same manner as in the reaction described in Step 2 of the aforementioned Method M.

Compound (XIV-1a), which is compound (XIV) (used as a starting material compound in the aforementioned Method L) wherein X is —X²—CH═CH— wherein X² is as defined above, and compound (XIV-1b), which is compound (XIV) wherein X is —X²—CH₂CH₂— wherein X² is as defined above, are produced, for example, according to the following Method X.

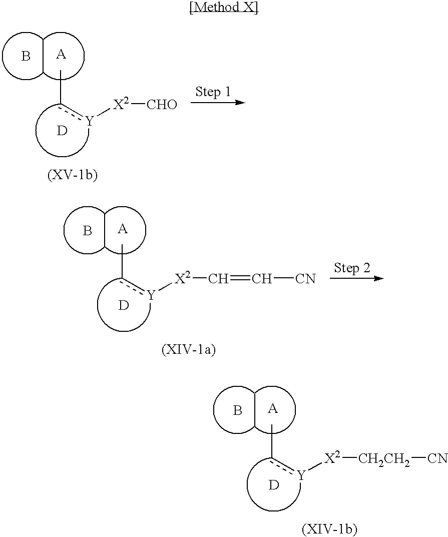

wherein each symbol is as defined above.

### [Step 1]

In this step, compound (XIV-1a) can be produced by reacting compound (XV-1b) with diethyl (cyanomethyl)phosphonate. This reaction is carried out in the same manner as in the reaction described in Step 1 of the aforementioned Method T2.

### [Step 2]

In this step, compound (XIV-1b) can be produced by subjecting compound (XIV-1a) to a hydrogenation reaction. This reaction is carried out in the same manner as in the reaction described in Step 2 of the aforementioned Method M.

Compound (XVIII-1a), which is compound (XVIII) (used as a starting material compound in the aforementioned Method O1, Method O2, Method P and Method Q) wherein R^{1a} is a hydrogen atom, is produced, for example, according to the following Method Y.

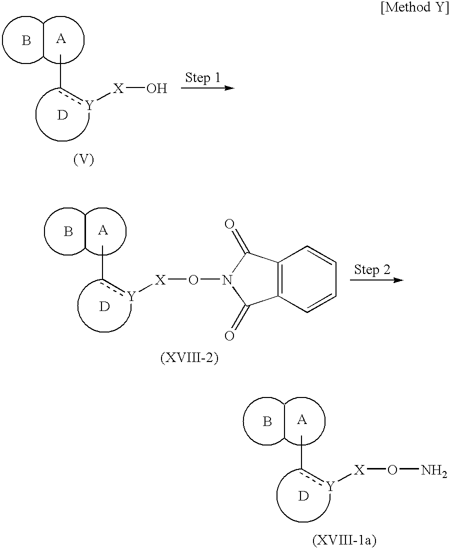

wherein each symbol is as defined above.

### [Step 1]

In this step, compound (XVIII-2) can be produced by reacting compound (V) with N-hydroxyphthalimide. This reaction is carried out in the same manner as in the reaction described in Step 2 of the aforementioned Method I.

### [Step 2]

In this step, compound (XVIII-1a) can be produced by subjecting compound (XVIII-2) to hydrolysis. This reaction is carried out in the same manner as in the reaction described in Step 2 of the aforementioned Method V2.

Compound (XV-1a) used as a starting material compound in the aforementioned Method N, Method T3, Method T4 and Method W, compound (XV) used as a starting material compound in the aforementioned Method M and Method U2, compound (XV-1b) used as a starting material compound in the aforementioned Method T2 and Method X, compound (XV-1f) used as a starting material compound in the below-mentioned Method AL, compound (XV-1h) used as a starting material compound in the below-mentioned Method AO, compound (XV-1k) used as a starting material compound in the below-mentioned Method AQ, and compound (XV-1m) used as a starting material compound in the below-mentioned Method AV, are produced, for example, according to the following Method Z1 to Method Z3.

Compound (XV-1e), which is compound (XV-1a) wherein ring D is bonded to the nitrogen atom on ring A, is produced, for example, according to the following Method Z1.

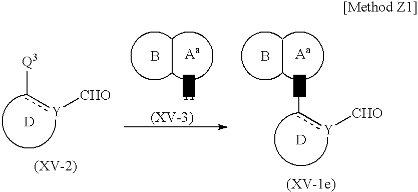

wherein Q³ is a halogen atom or trifluoromethylsulfonyloxy, ring A^{a} is a 5- to 7-membered monocycle containing NH, which is optionally substituted, and the other symbols are as defined above.

As the “5- to 7-membered monocycle containing NH, which is optionally substituted” for ring A^{a}, rings containing, as a ring-constituting member, at least one unsubstituted NH (—NH—) (e.g., pyrrole, pyrazole, imidazole), from among the aforementioned “5- to 7-membered monocycle containing NH, which is optionally substituted” for ring A, can be mentioned.

In this method, compound (XV-1e) can be produced by reacting compound (XV-2) with compound (XV-3). This reaction is carried out in the presence of a base, in a solvent that does not adversely influence the reaction. This reaction may be carried out, in the presence of an organic metal catalyst and a phosphine ligand, as necessary.

As the base, for example, alkali metal salts such as potassium hydroxide, sodium hydroxide, sodium hydrogencarbonate, sodium carbonate, potassium carbonate, cesium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like, and the like can be mentioned. These solvents may be used in a mixture at an appropriate ratio.

As the organic metal catalyst, palladium(II) acetate, tetrakis(triphenylphosphine)palladium(0), dichlorobis(triphenylphosphine)palladium(II) and the like can be mentioned.

As the phosphine ligand, 2,2′-bis(diphenylphosphino)-1,1′-binaphthyl (BINAP), tris(2-methylphenyl)phosphine, 1,1′-bis(diphenylphosphino)ferrocene and the like can be mentioned.

The amount of compound (XV-3) to be used is generally 1 to 20 mol, preferably 1 to 5 mol, per 1 mol of compound (XV-2).

The amount of the base to be used is generally 1 to 20 mol, preferably 1 to 10 mol, per 1 mol of compound (XV-2).

The amount of the organic metal catalyst to be used is generally 0.001 to 1 mol, preferably 0.01 to 0.5 mol, per 1 mol of compound (XV-2).

The amount of the phosphine ligand to be used is generally 0.001 to 1 mol, preferably 0.01 to 0.5 mol, per 1 mol of compound (XV-2).

The reaction temperature is generally −10 to 250° C., preferably 20 to 150° C.

The reaction time is generally 0.5 to 100 hr.

Compound (XV-2) can be produced, for example, according to the below-mentioned Method AR or a method analogous thereto. Compound (XV-3) can be produced according to a method known per se.

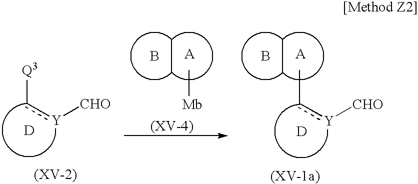

wherein Mb is a substituted boron atom when compound (XV-4) is an organic boronic acid or an organic boronate, or a substituted tin atom when compound (XV-4) is an organic tin reagent, and the other symbols are as defined above.

As the substituted boron atom for Mb, dihydroxyboryl group, 4,4,5,5-tetramethyl-1,3,2-dioxaborolan-2-yl group and the like can be mentioned.

As the substituted tin atom for Mb, trimethylstannyl group, tributylstannyl group and the like can be mentioned.

In this method, compound (XV-1a) can be produced by subjecting compound (XV-2) and compound (XV-4) to a coupling reaction using an organic metal catalyst. This reaction is carried out in the presence of a base, in a solvent that does not adversely influence the reaction, as necessary. This reaction may be carried out, in the presence of a phosphine ligand, as necessary.

As the organic metal catalyst, palladium(II) acetate, tetrakis(triphenylphosphine)palladium(0), tris(dibenzylideneacetone)dipalladium(0), dichlorobis(triphenylphosphine)palladium(II) and the like can be mentioned.

As the base, for example, alkali metal salts such as potassium hydroxide, sodium hydroxide, sodium hydrogencarbonate, sodium carbonate, potassium carbonate, cesium carbonate and the like; metal hydrides such as potassium hydride, sodium hydride and the like, and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; amides such as N,N-dimethylformamide, N,N-dimethylacetamide, N-methylpyrrolidone and the like; sulfoxides such as dimethylsulfoxide and the like; alcohols such as methanol, ethanol, isopropanol, tert-butyl alcohol and the like; water and the like can be mentioned. These solvents may be used in a mixture at an appropriate ratio.

The amount of compound (XV-4) to be used is generally 1 to 10 mol, preferably 1 to 5 mol, per 1 mol of compound (XV-2).

The amount of the organic metal catalyst to be used is generally 0.001 to 1 mol, preferably 0.01 to 0.5 mol, per 1 mol of compound (XV-2).

The amount of the base to be used is generally 1 to 20 mol, preferably 1 to 10 mol, per 1 mol of compound (XV-2).

As the phosphine ligand, 2,2′-bis(diphenylphosphino)-1,1′-binaphthyl (BINAP), tris(2-methylphenyl)phosphine, 1,1′-bis(diphenylphosphino)ferrocene, 2-dicyclohexylphosphino-2′,6′-dimethoxybiphenyl and the like can be mentioned.

The amount of the phosphine ligand to be used is generally 0.001 to 1 mol, preferably 0.01 to 0.5 mol, per 1 mol of compound (XV-2).

The reaction temperature is generally 0 to 200° C., preferably 50 to 150° C.

The reaction time is generally 0.5 to 50 hr.

Compound (XV-4) can be produced according to a method known per se.

Compound (XV) used as a starting material compound in the aforementioned Method M, and compound (XV-1b) used as a starting material compound in the aforementioned Method T2 and Method X, are produced, for example, according to the following Method Z3.

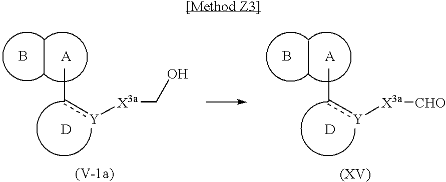

wherein each symbol is as defined above.

In this reaction, compound (XV) can be produced by subjecting compound (V-1a) to an oxidization reaction. This reaction is generally carried out in the presence of an oxidant, in a solvent that does not adversely influence the reaction.

As the oxidant, for example, metal oxidants such as manganese dioxide, pyridinium chlorochromate, pyridinium dichromate, ruthenium oxide and the like can be mentioned.

As the solvent that does not adversely influence the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like, and the like can be mentioned. These solvents may be used in a mixture at an appropriate ratio.

The amount of the oxidant to be used is generally 1 to 50 mol, preferably 1 to 10 mol, per 1 mol of compound (V-1a).

The reaction temperature is generally −50 to 150° C., preferably −10 to 100° C.

The reaction time is generally 0.5 to 50 hr.

Compound (V-1a) can be produced, for example, according to the aforementioned Method U1 or a method analogous thereto.

Compound (I-15), which is compound (I) wherein W is —CONR^{1a}S(O)ₘOR² wherein m is 2 and the other symbols are as defined above, is produced, for example, according to the following Method AA.

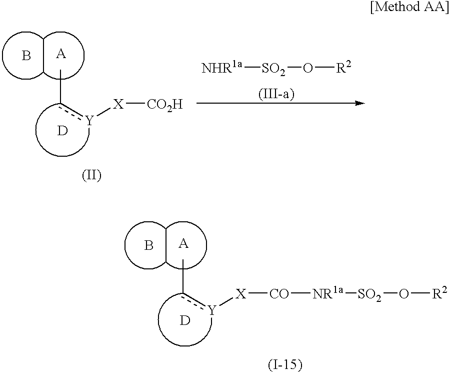

wherein each symbol is as defined above.

In this method, compound (I-15) can be produced by reacting compound (II) with compound (III-a). This reaction is carried out in the same manner as in the condensation reaction described in the aforementioned Method A1.

Compound (III-a) can be produced according to a method known per se.

Compound (I-16), which is compound (I) wherein W is —S(O)ₘNR^{1a}CONR^{1c}R² wherein each symbol is as defined above, is produced, for example, according to the following Method AB.

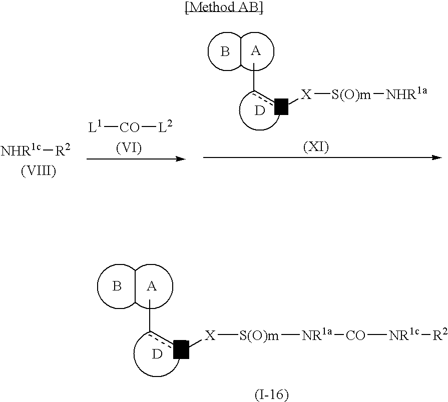

wherein each symbol is as defined above.

In this method, compound (I-16) can be produced from compound (XI). This reaction is carried out according to a method known per se, for example, by reacting compound (VIII) with compound (VI) in a solvent that does not adversely influence the reaction, at −10° C. to 120° C. for 0.5 to 10 hr, and reacting the obtained compound with compound (XI) in a solvent that does not adversely influence the reaction, at −10° C. to 120° C. for 0.5 to 50 hr. This reaction may be carried out in the presence of 1 to 20 mol of a base, per 1 mol of compound (XI), where necessary.

As compound (VI), for example, N,N′-carbonyldiimidazole, diphosgene, triphosgene and the like can be mentioned.

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned. These bases may be used in a mixture at an appropriate ratio.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, pyridine, water and the like can be mentioned. These solvents may be used in a mixture at an appropriate ratio.

The amount of compound (VI) to be used is generally 1 to 10 mol, preferably 1 to 5 mol, per 1 mol of compound (XI).

The amount of compound (VIII) to be used is generally 1 to 10 mol, preferably 1 to 5 mol, per 1 mol of compound (XI).

Compound (I-7d), which is compound (I-7a) (compound (I) wherein W is —S(O)ₘNR^{1a}COₙR² wherein n is 1 and the other symbols are as defined above) wherein R² is a non-aromatic heterocyclic group containing NH, is produced, for example, according to the following Method AC.

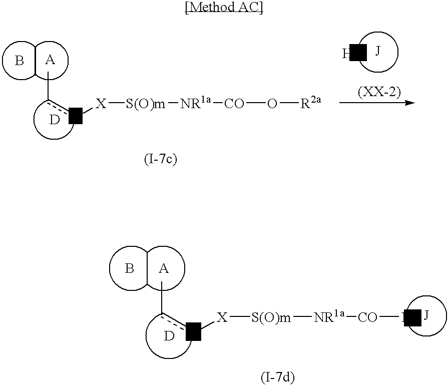

wherein R^{2a} is a C₁₋₆ alkyl group, ring J is a non-aromatic heterocycle containing NH, and the other symbols are as defined above.

The “C₁₋₆ alkyl group” for R^{2a} is preferably ethyl, propyl or butyl.

As the “non-aromatic heterocycle containing NH” for ring J, pyrrolidine, morpholine, piperizine and the like can be mentioned.

In this method, compound (I-7d) can be produced by reacting compound (I-7c) with compound (XX-2). This reaction is carried out in a solvent that does not adversely influence the reaction.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, pyridine, water and the like can be mentioned. These solvents may be used in a mixture at an appropriate ratio.

This reaction may be carried out in the presence of 1 to 5 mol of a base, per 1 mol of compound (I-7c), as necessary.

As the base that does not adversely influence the reaction, those exemplified in the aforementioned Method AB can be mentioned.

The amount of compound (XX-2) to be used is generally 1 to 10 mol, preferably 1 to 5 mol, per 1 mol of compound (I-7c).

The reaction temperature is generally −30° C. to 150° C.

The reaction time is generally 0.5 to 30 hr.

Compound (I-7c) can be produced, for example, according to the below-mentioned Method AU or a method analogous thereto. Compound (XX-2) can be produced according to a method known per se.

Compound (I-17a), which is compound (I) wherein W is —NR^{1b}S(O)ₘNR^{1a}COₙR² wherein m is 2, n is 1 and the other symbols are as defined above, is produced, for example, according to the following Method AD.

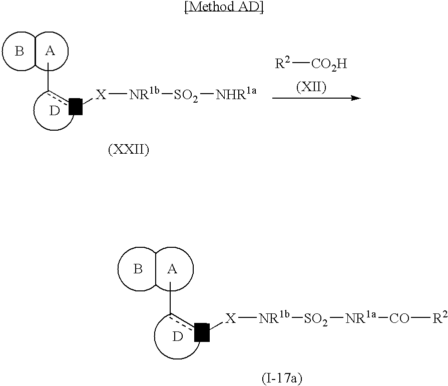

wherein the each symbol is as defined above.

In this method, compound (I-17a) can be produced by reacting compound (XXII) with compound (XII). This reaction is carried out in the same manner as in the condensation reaction described in the aforementioned Method A1.

Compound (XXII) can be produced, for example, according to the below-mentioned Method AS or a method analogous thereto.

Compound (I-17b), which is compound (I) wherein W is —NR^{1b}S(O)ₘNR^{1a}COₙR² wherein m is 2, n is 2 and the other symbols are as defined above, is produced, for example, according to the following Method AE.

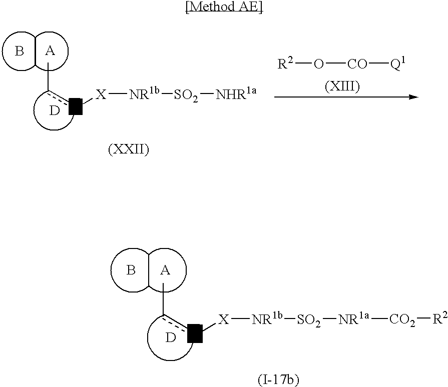

wherein the each symbol is as defined above.

In this method, compound (I-17b) can be produced by reacting compound (XXII) with compound (XIII). This reaction is carried out in the same manner as in the condensation reaction described in the aforementioned Method A1.

Compound (I-18b), which is compound (I) wherein ring A is pyrrole bonded to ring D at the 1-position and having a halogen atom at the 3-position, is produced, for example, according to the following Method AF.

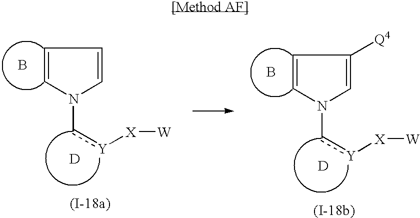

wherein Q⁴ is a halogen atom, and the other symbols are as defined above.

The “halogen atom” for Q⁴ is preferably a chlorine atom or a bromine atom.

In this method, compound (I-18b) can be produced by reacting compound (I-18a) with a halogenating agent. This reaction is carried out in a solvent that does not adversely influence the reaction.

As the halogenating agent, N-chlorosuccinimide, N-bromosuccinimide and the like can be mentioned.

The amount of the halogenating agent to be used is generally 1 to 10 mol, per 1 mol of compound (I-18a).

The reaction temperature is generally −10° C. to 150° C., preferably 0 to 80° C.

The reaction time is 0.5 to 50 hr.

As the solvent that does not adversely influence the reaction, for example, aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethyl sulfoxide and the like; acetonitrile and the like can be mentioned. These solvents may be used in a mixture at an appropriate ratio.

Compound (I-18a) can be produced, for example, according to the aforementioned Method A1, Method B to Method G, Method H1, Method H2, Method I to Method N, Method O1, Method O2, Method P to Method R, Method S1, Method S2, Method AA to Method AE, the below-mentioned Method AG to Method AL, Method AU or a method analogous thereto.

Compound (I-19b), which is compound (I) having a hydroxyl group on ring B, compound (I-19c), which is compound (I) having an optionally substituted C₁₋₆ alkoxy group on ring B, and compound (I-19d), which is compound (I) having an optionally substituted C₁₋₆ alkylsulfonyloxy group on ring B, are produced, for example, according to the following Method AG.

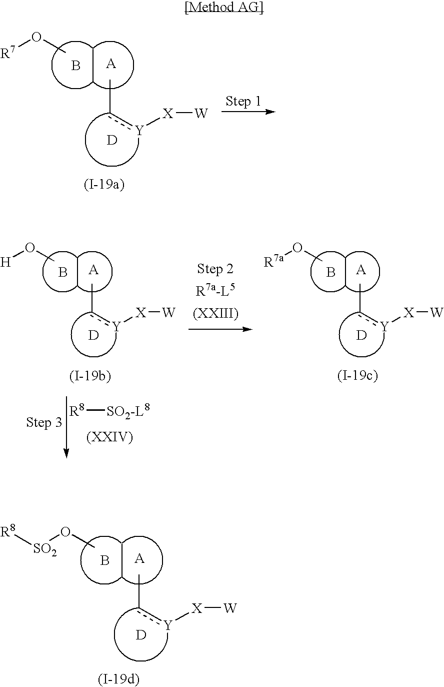

wherein R⁷ is methyl or benzyl, R^{7a} is an optionally substituted C₁₋₆ alkyl group, R⁸ is a C₁₋₆ alkyl group, L⁵ and L⁶ are the same or different and each is a leaving group, and the other symbols are as defined above.

As the “leaving group” for L⁵ or L⁶, those exemplified for the aforementioned L¹ or L² can be mentioned.

As the substituent of the “optionally substituted C₁₋₆ alkyl group” for R^{7a}, 1 to 3 substituents selected from (a) a C₆₋₁₄ aryl group, (b) a C₁₋₆ alkoxy group, (c) a C₃₋₁₀ cycloalkyl group and (d) a C₁₋₆ alkyl-carbonyl group can be mentioned.

### [Step 1]

In this step, compound (I-19b) can be produced from compound (I-19a).

When R⁷ is methyl or benzyl, the reaction is carried out in the presence of boron tribromide, in a solvent that does not adversely influence the reaction.

As the solvent that does not adversely influence the reaction, halogenated hydrocarbons such as dichloromethane and the like, and the like can be mentioned.

The amount of the boron tribromide to be used is generally 1 to 20 mol, per 1 mol of compound (I-19a).

The reaction temperature is generally −100 to 150° C., preferably −80 to 100° C.

The reaction time is generally 0.1 to 30 hr.

When R⁷ is benzyl, the reaction can be carried out in the presence of a metal catalyst such as palladium-carbon, palladium black, palladium chloride, platinum oxide, palladium black, platinum-palladium, Raney-nickel, Raney-cobalt and the like and a hydrogen source, or in the presence of an acid, in a solvent that does not adversely influence the reaction.

The amount of the metal catalyst to be used is generally 0.001 to 1000 mol, preferably 0.01 to 100 mol, per 1 mol of compound (I-19a).

As the hydrogen source, for example, hydrogen gas, formic acid, an amine salt of formic acid, phosphinate, hydrazine and the like can be mentioned.

As the acid, for example, organic acids such as trifluoroacetic acid and the like can be mentioned.

The amount of the acid to be used is generally 0.01 to 1000 mol, preferably 0.1 to 100 mol, per 1 mol of compound (I-19a).

As the solvent that does not adversely influence the reaction, for example, alcohols such as methanol, ethanol, propanol, 2-propanol, 2-methoxyethanol, butanol, isobutanol, tert-butanol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as dichloromethane, chloroform, 1,2-dichloroethane, 1,1,2,2-tetrachloroethane and the like; amides such as N,N-dimethylformamide, N,N-dimethylacetamide, N-methylpyrrolidone and the like; ethyl acetate; acetic acid and the like can be mentioned. These solvents may be used in a mixture at an appropriate ratio.

The reaction temperature is generally 0 to 150° C., preferably 10 to 80° C.

The reaction time is generally 0.5 to 100 hr.

Compound (I-19a) can be produced, for example, according to the aforementioned Method A1, Method B, Method J, Method K, Method L, Method R, Method AA, Method AB or the below-mentioned Method AU, or a method analogous thereto.

### [Step 2]

In this step, compound (I-19c) can be produced by reacting compound (I-19b) with compound (XXIII). This reaction is generally carried out in the presence of a base, in a solvent that does not adversely influence. The reaction efficiency can be improved by using sodium iodide, as necessary.

The amount of compound (XXIII) to be used is generally 1 to 20 mol, per 1 mol of compound (I-19b).

As the base, for example, alkali metal salts such as potassium hydroxide, sodium hydroxide, sodium hydrogencarbonate, sodium carbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

The amount of the base to be used is generally 1 to 20 mol, per 1 mol of compound (I-19b).

The amount of the sodium iodide to be used is generally 1 to 20 mol, preferably 1 to 10 mol, per 1 mol of compound (I-19b).

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethyl sulfoxide and the like, acetone, acetnitrile and the like can be mentioned. These solvents may be used in a mixture at an appropriate ratio.

The reaction temperature is generally −30 to 150° C., preferably −10 to 100° C.

The reaction time is generally 0.5 to 100 hr.

Compound (XXIII) can be produced according to a method known per se.

### [Step 3]

In this step, compound (I-19d) can be produced by reacting compound (I-19b) with compound (XXIV). This reaction is generally carried out in the presence of a base, in a solvent that does not adversely influence.

The amount of compound (XXIV) to be used is generally 1 to 20 mol, preferably 1 to 10 mol, per 1 mol of compound (I-19b).

As the base, for example, alkali metal salts such as potassium hydroxide, sodium hydroxide, sodium hydrogencarbonate, sodium carbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

The amount of the base to be used is generally 1 to 20 mol, preferably 1 to 5 mol, per 1 mol of compound (I-19b).

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethyl sulfoxide and the like, and the like can be mentioned. These solvents may be used in a mixture at an appropriate ratio.

The reaction temperature is generally −80 to 150° C., preferably −10 to 100° C.

The reaction time is generally 0.5 to 100 hr.

Compound (XXIV) can be produced according to a method known per se.

Compound (I-20b), which is compound (I) having a C₆₋₁₄ aryl group, an aromatic heterocyclic group or a C₃₋₁₀ cycloalkyl group on ring B, is produced, for example, according to the following Method AH.

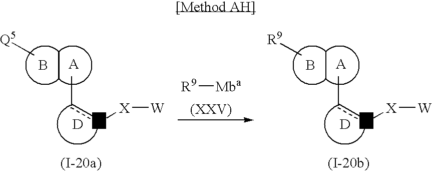

wherein Q⁵ is a halogen atom, Mb^{a} is a substituted boron atom when compound (XXV) is an organic boronic acid or an organic boronate, or a substituted tin atom when compound (XXV) is an organic tin reagent, R⁹ is a C₆₋₁₄ aryl group, an aromatic heterocyclic group or a C₃₋₁₀ cycloalkyl group, and the other symbols are as defined above.”

The “halogen atom” for Q⁵ is preferably a bromine atom or an iodine atom.

As the “substituted boron atom” or “substituted tin atom” for Mb^{a}, those exemplified for the aforementioned Mb can be mentioned.

In this method, compound (I-20b) can be produced by subjecting compound (I-20a) and compound (XXV) to a coupling reaction using an organic metal catalyst. This reaction is carried out in the same manner as in the reaction described in the aforementioned Method Z2.

Compound (I-20a) can be produced, for example, according to the aforementioned Method A1, Method B to Method G, Method H1, Method H2, Method I to Method N, Method O1, Method O2, Method P to Method R, Method S1, Method S2, Method AA to Method AG, Method AI to Method AL, Method AU or a method analogous thereto. Compound (XXV) can be produced according to a method known per se.

Compound (I-Id), which is compound (I-1) (compound (I) wherein W is —CONR^{1a}S(O)ₘR² wherein each symbol is as defined above) wherein R² is 4-oxopiperidin-1-yl group), compound (1-1e), which is compound (I-1) wherein R² is 4-hydroxypiperidin-1-yl group, and compound (I-1f), which is compound (I-1) wherein R² is 4-hydroxy-4-methylpiperidin-1-yl group, are produced, for example, according to the following Method AI.

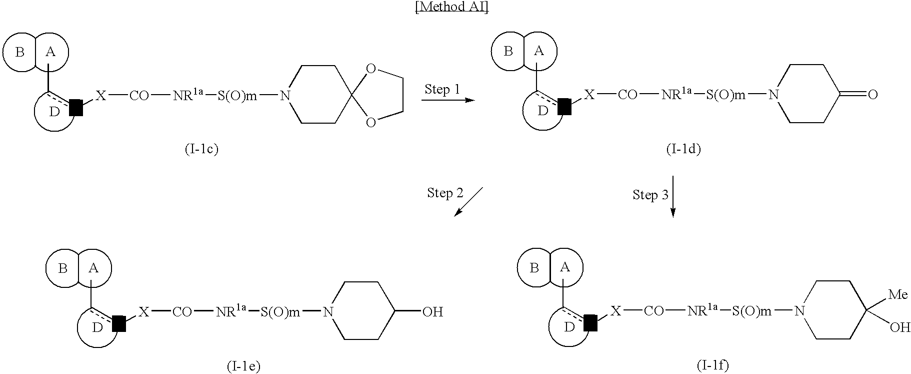

wherein the each symbol is as defined above.

### [Step 1]

In this step, compound (I-1d) can be produced by subjecting compound (I-1c) to a deketalation reaction. This reaction is carried out in the same manner as in the reaction described in Step 2 of the aforementioned Method T5.

Compound (I-1c) can be produced, for example, according to the aforementioned Method A1 or a method analogous thereto.

### [Step 2]

In this step, compound (I-1e) can be produced by subjecting compound (I-1d) to a reduction reaction. This reaction is carried out in the same manner as in the reaction described in Step 4 of the aforementioned Method N.

### [Step 3]

In this step, compound (I-1f) can be produced by reacting compound (I-1d) with a methylating agent. This reaction is carried out in a solvent that does not adversely influence the reaction.

As the methylating agent, methylmagnesium chloride, methyl magnesium bromide, methyllithium and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like, and the like can be mentioned. These solvents may be used in a mixture at an appropriate ratio.

The reaction temperature is generally −80 to 150° C., preferably −10 to 80° C.

The reaction time is generally 0.1 to 30 hr.

Compound (I-1h), which is compound (I-1) (compound (I) wherein W is —CONR^{1a}S(O)ₘR² wherein each symbol is as defined above) wherein R² is an optionally substituted hydroxyphenyl group, is produced, for example, according to the following Method AJ.

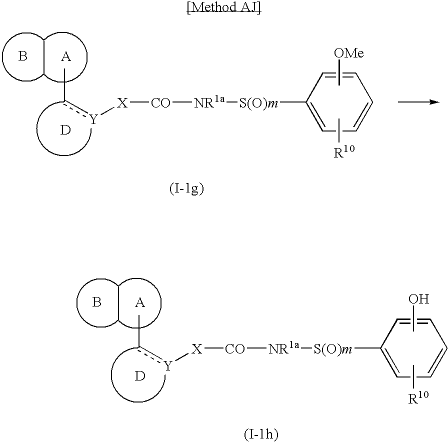

wherein R¹⁰ is an optionally substituted C₁₋₆ alkyl group, and the other symbols are as defined above.

As the substituents of the “optionally substituted C₁₋₆ alkyl group” for R¹⁰, 1 to 3 halogen atoms (preferably a fluorine atom) can be mentioned.

In this method, compound (I-1 h) can be produced by S reacting compound (I-1g) with boron tribromide. This reaction is carried out in a solvent that does not adversely influence the reaction.

As the solvent that does not adversely influence the reaction, halogenated hydrocarbons such as dichloromethane and the like, and the like can be mentioned.

The amount of the boron tribromide to be used is generally 1 to 20 mol, per 1 mol of compound (I-1g).

The reaction temperature is generally −100 to 150° C., preferably −80 to 100° C.

The reaction time is generally 0.1 to 50 hr.

Compound (I-1g) can be produced, for example, according to the aforementioned Method A1 or a method analogous thereto.

Compound (I-2d), which is compound (I-2a) (compound (I) wherein W is —CONR^{1a}S(O)ₘNR^{1c}R² wherein m is 2 and the other symbols are as defined above) wherein NR^{1c}R² is (3-hydroxy-3-methylbutyl)amino group, is produced, for example, according to the following Method AK.

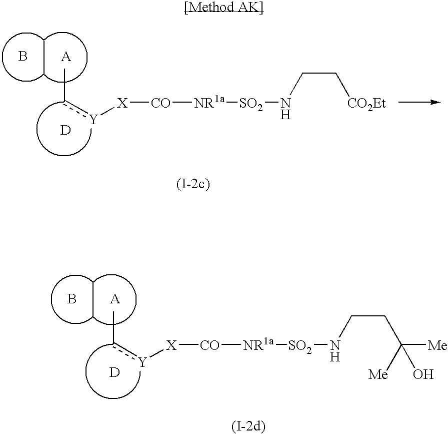

wherein the each symbol is as defined above.

In this method, compound (I-2d) can be produced by subjecting compound (I-2c) to a dimethylation reaction. This reaction is carried out in a solvent that does not adversely influence the reaction.

As the methylating agent, methylmagnesium chloride, methylmagnesium bromide, methyllithium and the like can be mentioned.

The amount of the methylating agent to be used is generally 2-20 mol, preferably 2 to 10 mol, per 1 mol of compound (I-2c).

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like, and the like can be mentioned. These solvents may be used in a mixture at an appropriate ratio.

The reaction temperature is generally −80 to 100° C., preferably −10 to 60° C.

The reaction time is generally 0.1 to 30 hr.

Compound (I-2c) can be produced, for example, according to the aforementioned Method B or a method analogous thereto.

Compound (I-1i), which is compound (I-1) (compound (I) wherein W is —CONR^{1a}S(O)ₘR² wherein each symbol is as defined above) wherein X is —CH═CH— and ring D is pyrazole (bonded to ring A at the 3-position and bonded to X at the 4-position) having tert-butoxycarbonyl group at the 1-position, and compound (I-1j), which is compound (I-1) wherein X is —CH═CH— and ring D is pyrazole (bonded to ring A at the 3-position and bonded to X at the 4-position) having no substituents at the 1- and 2-positions, are produced, for example, according to the following Method AL.

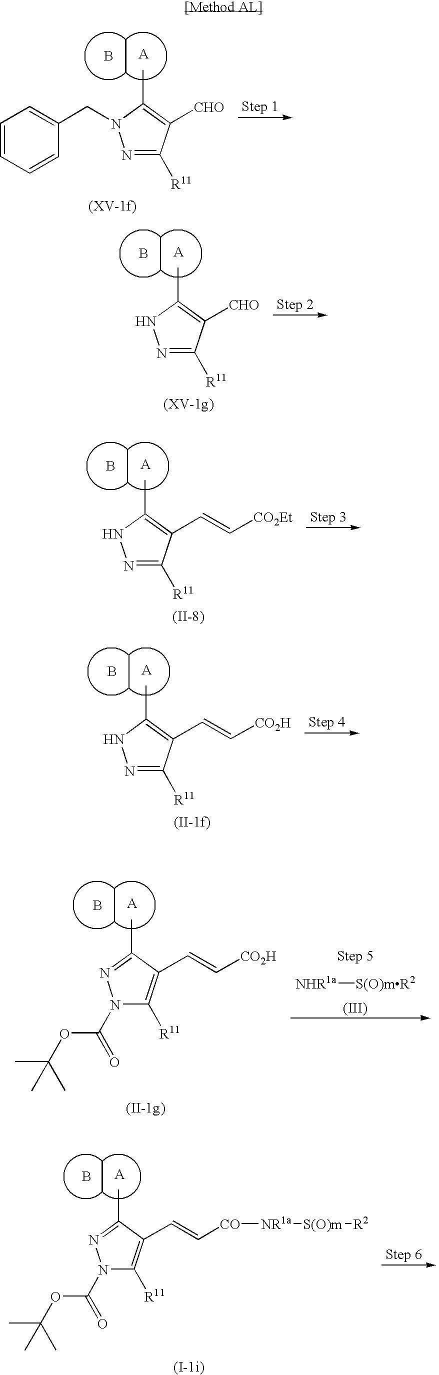

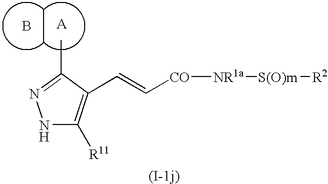

wherein R¹¹ is an optionally substituted C₁₋₆ alkyl group or a C₁₋₆ cycloalkyl group, and the other symbols are as defined above.”

As the substituents of the “optionally substituted C₁₋₆ alkyl group” for R¹¹, 1 to 3 halogen atoms (preferably a fluorine atom) and a C₁₋₆ alkoxy group can be mentioned.

### [Step 1]

In this step, compound (XV-1g) can be produced by subjecting compound (XV-1f) to a debenzylation reaction. This reaction is carried out in trifluoroacetic acid at 0° C. to 80° C. for 1 to 200 hr.

The amount of the trifluoroacetic acid to be used is generally 5 to 1000 mol, per 1 mol of compound (XV-1f).

Compound (XV-1f) can be produced, for example, according to the aforementioned Method Z1, Method Z2, the below mentioned Method AO, Method AQ or a method analogous thereto.

### [Step 2]

In this step, compound (II-8) can be produced by reacting compound (XV-1g) with ethyl (triphenylphosphoranylidene)acetate. This reaction is carried out in a solvent that does not adversely influence the reaction.

The amount of the ethyl (triphenylphosphoranylidene)acetate to be used is generally 1 to 20 mol, preferably 1 to 5 mol, per 1 mol of compound (XV-1g).

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like, acetonitrile and the like can be mentioned. These solvents may be used in a mixture at an appropriate ratio.

The reaction temperature is generally −10 to 150° C., preferably 10 to 120° C.

The reaction time is generally 0.5 to 50 hr.

### [Step 3]

In this step, compound (II-1f) can be produced by subjecting compound (II-8) to hydrolysis. This reaction is carried out in the same manner as in the reaction described in the aforementioned Method T1.

### [Step 4]

In this step, compound (II-1g) can be produced by reacting compound (TI-1f) with di-tert-butyl dicarbonate. This reaction is carried out in the presence of a base, in a solvent that does not adversely influence the reaction.

The amount of the di-tert-butyl dicarbonate to be used is generally 1 to 20 mol, per 1 mol of compound (II-1f).

As the base, for example, alkali metal salts such as potassium hydroxide, sodium hydroxide, sodium hydrogencarbonate, sodium carbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

The amount of the base to be used is generally 1 to 10 mol, per 1 mol of compound (II-1f).

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; acetonitrile, ethyl acetate, water and the like can be mentioned. These solvents may be used in a mixture at an appropriate ratio.

The reaction temperature is generally 0 to 150° C., preferably 10 to 80° C.

The reaction time is generally 0.5 to 100 hr.

### [Step 5]

In this step, compound (I-1i) can be produced by reacting compound (II-1g) with compound (III). This reaction is carried out in the same manner as in the reaction described in the aforementioned Method A1.

### [Step 6]

In this step, compound (I-1j) can be produced by subjecting compound (I-1i) to deprotection. This reaction is carried out in the same manner as in the reaction described in Step 3 of the aforementioned Method S1.

Compound (II-9b), which is compound (II-2) wherein ring A is pyrrole bonded to ring D at the 1-position and having a halogen atom at the 3-position, is produced, for example, according to the following Method AM.

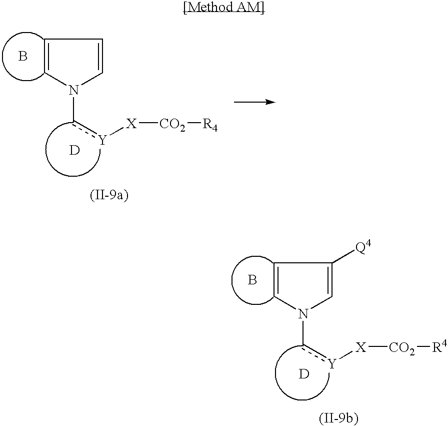

wherein the each symbol is as defined above.

In this method, compound (II-9b) can be produced by reacting compound (II-9a) with a halogenating agent. This reaction is carried out in the same manner as in the reaction described in the aforementioned Method AF.

Compound (II-9a) can be produced, for example, according to Step 1 or Step 2 of the aforementioned Method T2, the below-mentioned Method AN, Method AP or a method analogous thereto.

Compound (II-10b), which is compound (II-2) wherein X is cyclopropane ring, is produced, for example, according to the following Method AN.

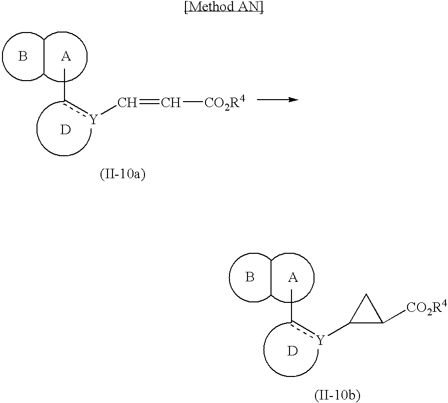

wherein the each symbol is as defined above.

In this method, compound (II-10b) can be produced by subjecting compound (II-10a) to a cyclopropanation reaction using a base or an organic metal catalyst.

The cyclopropanation reaction using a base is carried out using a cyclopropanating agent, in the presence of a base, in a solvent that does not adversely influence the reaction.

As the cyclopropanating agent, trimethylsulfoxonium iodide, methyltriphenylphosphonium bromide, nitromethane and the like can be mentioned.

As the base, for example, alkali metal salts such as potassium hydroxide, sodium hydroxide, sodium carbonate, potassium carbonate and the like; amines such as pyridine, triethylamine, tributylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like; organic metals such as methyllithium, butyllithium and the like; alkali metal fluorides such as cesium fluoride, potassium fluoride and the like, and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, 1,2-dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethylsulfoxide and the like; nitriles such as acetonitrile, propionitrile and the like, and the like can be mentioned. These solvents may be used in a mixture at an appropriate ratio.

The reaction temperature is generally −70 to 150° C., preferably −20 to 80° C.

The reaction time is generally 1 to 100 hr, preferably 1 to 60 hr.

The amount of the cyclopropanating agent to be used is generally 1 to 50 mol, preferably 1 to 5 mol, per 1 mol of compound (II-10a).

The amount of the base to be used is generally 1 to 50 mol, preferably 1 to 5 mol, per 1 mol of compound (II-10a).

The cyclopropanation reaction using an organic metal catalyst is carried out using a diazoalkane in a solvent that does not adversely influence the reaction, in the presence of a ligand, as necessary.

As the organic metal catalyst, for example, palladium(II) acetate, coppertriflate(I), rhodium(II), acetate dimer and the like can be mentioned.

As the diazoalkane, diazomethane and the like can be mentioned.

As the ligand, 2,2′-diisopropylidenebis[(4S)-4-tert-butyl-2-oxazoline] and the like can be mentioned.

As the solvent that does not adversely influence the reaction, for example, aliphatic hydrocarbons such as hexane, heptane and the like; ethers such as diethyl ether, diisopropyl ether, tert-butyl methyl ether, tetrahydrofuran, dioxane, dimethoxyethane and the like; halogenated hydrocarbons such as dichloromethane, chloroform, 1,2-dichloroethane, 1,1,2,2-tetrachloroethane and the like, and the like can be mentioned. These solvents may be used in a mixture at an appropriate ratio.

The reaction temperature is generally −70 to 150° C., preferably −20 to 80° C.

The reaction time is generally 0.1 to 100 hr, preferably 0.1 to 40 hr.

The amount of the organic metal catalyst to be used is generally 0.01 to 2 mol, preferably 0.01 to 0.5 mol, per 1 mol of compound (II-10a).

The amount of the diazoalkane to be used is generally 1 to 50 mol, preferably 1 to 5 mol, per 1 mol of compound (II-10a).

The amount of the ligand to be used is generally 0.01 to 2 mol, preferably 0.01 to 0.5 mol, per 1 mol of compound (II-10a).

Compound (II-10a) can be produced, for example, according to Step 1 of the aforementioned Method T2 or a method analogous thereto.

Compound (XV-1j), which is compound (XV) wherein ring A is a N-substituted pyrrole bonded to ring D at the 3-position, is produced for example, according to the following Method AO.

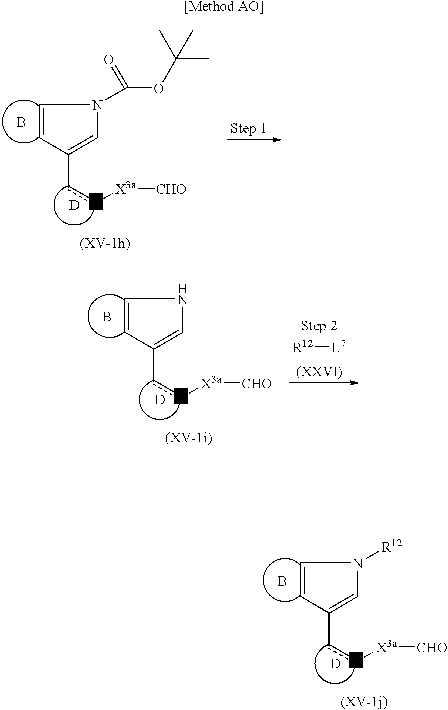

wherein R¹² is a C₁₋₆ alkyl group, L⁷ is a leaving group, and the other symbols are as defined above.

As the leaving group for L⁷, those exemplified for the aforementioned L¹ or L² can be mentioned.

### [Step 1]

In this step, compound (XV-1i) can be produced by subjecting compound (XV-1 h) to deprotection. This reaction is carried out in the same manner as in the reaction described in Step 3 of the aforementioned Method S1.

Compound (XV-1h) can be produced, for example, according to the aforementioned Method Z2 or a method analogous thereto.

### [Step 2]

In this step, compound (XV-1j) can be produced by reacting compound (XV-1i) with compound (XXVI). This reaction is carried out in the same manner as in the reaction described in Step 2 of the aforementioned Method AG.

Compound (XXVI) can be produced according to a method known per se.

Compound (II-11c), which is compound (II-2) having an optionally substituted C₁₋₆ alkoxy group on ring B, and compound (II-11g), which is compound (II-2) having 2-methoxy-1-methylethoxy group on ring B, are produced, for example, according to the following Method AP.

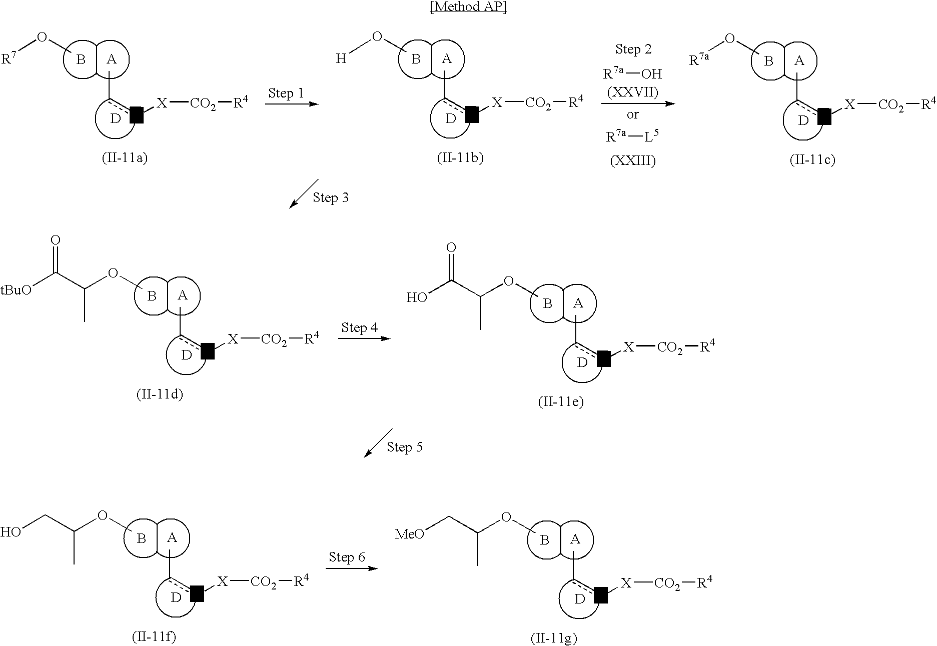

wherein the each symbol is as defined above.

### [Step 1]

In this step, compound (II-11b) can be produced from compound (II-11a). This reaction is carried out in the same manner as in the reaction described in Step 1 of the aforementioned Method AG.

Compound (II-11a) can be produced, for example, according to Step 1 or Step 2 of the aforementioned Method T2, Method AM, Method AN or a method analogous thereto.

### [Step 2]

In this step, compound (II-11c) can be produced by reacting compound (II-11b) with compound (XXVII) or compound (XXIII).

The reaction of compound (II-11b) with compound (XXVII) is carried out in the same manner as in the reaction described in Step 2 of the aforementioned Method I.

The reaction of compound (II-11b) with compound (XXIII) is carried out in the same manner as in the reaction described in Step 2 of the aforementioned Method AG.

Compound (XXVII) can be produced according to a method known per se.

### [Step 3]

In this step, compound (II-11d) can be produced by reacting compound (II-11b) with tert-butyl 2-bromopropionate. This reaction is carried out in the same manner as in the reaction described in Step 2 of the aforementioned Method AG.

### [Step 4]

In this step, compound (II-11e) can be produced by subjecting compound (II-11d) to hydrolysis. This reaction is carried out in the same manner as in the reaction described in the aforementioned Method T1.

### [Step 5]

In this step, compound (II-11f) can be produced from compound (II-11e). This reaction is carried out for example, by subjecting compound (II-11e) to halogenation at −10° C. to 100° C. for 0.5 to 30 hr, and subjecting the obtained compound to a reduction reaction at −10° C. to 100° C. for 0.1 to 50 hr.

The halogenation is carried out in the same manner as in the reaction described in the aforementioned Step 5 of Method N or the halogenation in the aforementioned Method A1.

The reduction reaction of the compound obtained by the halogenation is carried out in the same manner as in the reaction described in Step 4 of the aforementioned Method N.

### [Step 6]

In this step, compound (II-11g) can be produced by reacting compound (II-11f) with methyl iodide. This reaction is carried out in the same manner as in the reaction described in Step 2 of the aforementioned Method AG.

Compound (XV-1L), which is compound (XV) wherein ring A is 2,3-dihydropyrrole bonded to ring D at the 1-position, is produced, for example, according to the following Method AQ.

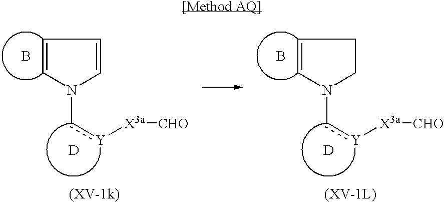

wherein the each symbol is as defined above.

In this method, compound (XV-1L) can be produced by subjecting compound (XV-1k) to a hydrogenation reaction. This reaction is carried out in the same manner as in the reaction described in Step 2 of the aforementioned Method M.

Compound (XV-1k) can be produced, for example, according to Step 2 of the aforementioned Method T4, the aforementioned Method Z1, Method Z3 or a method analogous thereto.

Of compound (XV-2), compound (XV-2a) represented by the formula:

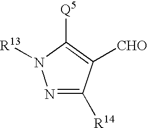

wherein R¹³ is C₁₋₆ alkyl group optionally substituted by C₆₋₁₄ aryl group(s), R¹⁴ is a C₁₋₆ alkyl group optionally substituted by 1 to 3 substituents selected from (a) a halogen atom and (b) a C₁₋₆ alkoxy group, or a C₃₋₁₀ cycloalkyl group, Q⁵ is a chlorine atom, a bromine atom or an iodine atom, and the other symbols are as defined above, can be produced, for example, according to the following Method AR.

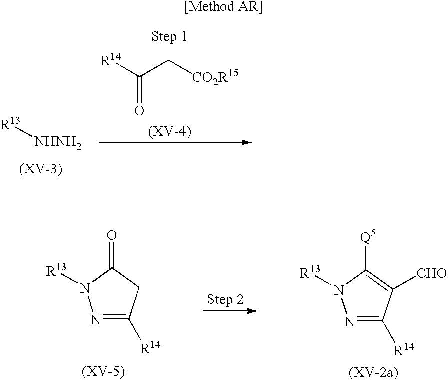

wherein R¹⁵ is a C₁₋₁₀ alkyl group, a benzyl group optionally substituted by C₁₋₆ alkyl group(s), or a C₆₋₁₄ aryl group optionally substituted by C₁₋₆ alkyl group(s), and the other symbols are as defined above.

R¹⁵ is preferably methyl, ethyl, tert-butyl, benzyl, phenyl or the like.

### [Step 1]

In this step, compound (XV-5) can be produced by reacting compound (XV-3) with compound (XV-4). This reaction is carried out in a solvent that does not adversely influence the reaction.

The amount of the compound (XV-3) to be used is generally 0.1 to 10 mol, preferably 0.5 to 5 mol, per 1 mol of compound (XV-4).

As the solvent that does not adversely influence the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethylsulfoxide and the like; ketones such as acetone, 2-butanone and the like; water and the like can be mentioned. These solvents may be used in a mixture at an appropriate ratio.

The reaction temperature is generally −80 to 200° C., preferably 0 to 150° C.

The reaction time is generally 0.5 to 100 hr.

Compound (XV-3) and compound (XV-4) can be produced according to a method known per se.

### [Step 2]

In this step, compound (XV-2a) can be produced by reacting compound (XV-5) with N,N-dimethylformamide and a phosphorus oxyhalide compound. This reaction is carried out without a solvent or in a solvent that does not adversely influence the reaction.

The amount of the N,N-dimethylformamide to be used is generally 1 to 20 mol, per 1 mol of compound (XV-5).

As the phosphorus oxyhalide compound, for example, phosphorus oxychloride, phosphorus oxybromide and the like can be mentioned.

The amount of the phosphorus oxyhalide compound to be used is generally 1 to 20 mol, per 1 mol of compound (XV-5).

As the solvent that does not adversely influence the reaction, for example, halogenated hydrocarbons such as chloroform, dichloromethane and the like; aromatic hydrocarbons such as nitrobenzene and the like; amides such as N,N-dimethylformamide and the like, and the like can be mentioned. These solvents may be used in a mixture at an appropriate ratio.

The reaction temperature is generally −80 to 200° C., preferably 0 to 150° C.

The reaction time is generally 0.5 to 30 hr.

Compound (XXII-1), which is compound (XXII) wherein X is —X^{3a}—CH₂— wherein X^{3a} is as defined above, and R^{1b} is a hydrogen atom, is produced, for example, according to the following Method AS.

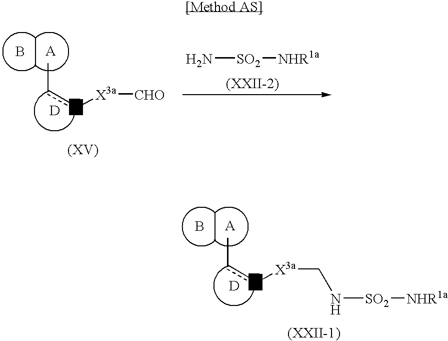

wherein the each symbol is as defined above.

In this method, compound (XXII-1) can be produced by subjecting compound (XV) to a reductive amination reaction with compound (XXII-2). This reaction is carried out by subjecting compound (XV) to an imination reaction with compound (XXII-2) in a solvent that does not adversely influence the reaction, at −100° C. to 100° C. for 0.1 to 30 hr, and subjecting the obtained compound to a reduction reaction at −100° C. to 100° C. for 0.1 to 50 hr.

The imination reaction may be carried out in the presence of an acid or a base.

The amount of compound (XXII-2) to be used is generally 1 to 10 mol, per 1 mol of compound (XV).

As the acid, for example, mineral acids such as hydrochloric acid, sulfuric acid and the like; Lewis acids such as boron trichloride, boron tribromide and the like; organic acids such as acetic acid, trifluoroacetic acid, p-toluenesulfonic acid and the like, and the like can be mentioned.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene, piperidine and the like; metal hydrides such as potassium hydride, sodium hydride and the like; alkali metal C₁₋₆ alkoxides such as sodium methoxide, sodium ethoxide, potassium tert-butoxide and the like, and the like can be mentioned.

The amount of the acid or base to be used is generally 0.1 to 50 mol, preferably 0.5 to 20 mol, per 1 mol of compound (XV), respectively.

As the solvent that does not adversely influence the reaction, for example, alcohols such as methanol, ethanol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, dimethoxyethane and the like; halogenated hydrocarbons such as chloroform, dichloromethane and the like, and the like can be mentioned. These solvents may be used in a mixture at an appropriate ratio.

The reduction reaction of the compound obtained by the imination reaction is carried out in the same manner as in the reaction described in Step 4 of the aforementioned Method N.

Compound (XXII-2) can be produced according to a method known per se.

Compound (1V-1), which is compound (IV) wherein R^{1a} is a hydrogen atom, is produced, for example, according to the following Method AT.

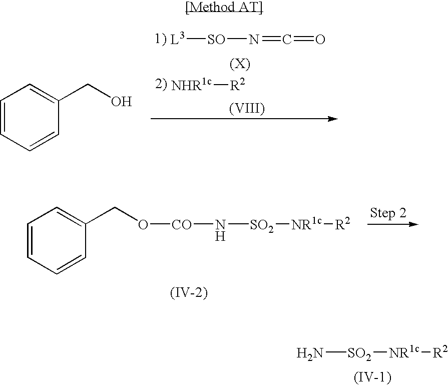

wherein the each symbol is as defined above.

### [Step 1]

In this step, compound (IV-2) can be produced by reacting benzyl alcohol with compound (X) and compound (VIII) successively. This reaction is carried out in the same manner as in the reaction described in Step 1 of the aforementioned Method I.

### [Step 2]

In this step, compound (IV-1) can be produced by subjecting compound (IV-2) to a hydrogenation reaction. This reaction is carried out in the same manner as in the reaction described in Step 2 of the aforementioned Method M.

Compound (I-7b), which is compound (I) wherein W is —S(O)ₘNR^{1a}COₙR² wherein n is 2 and the other symbols are as defined above, is produced, for example, according to the following Method AU.

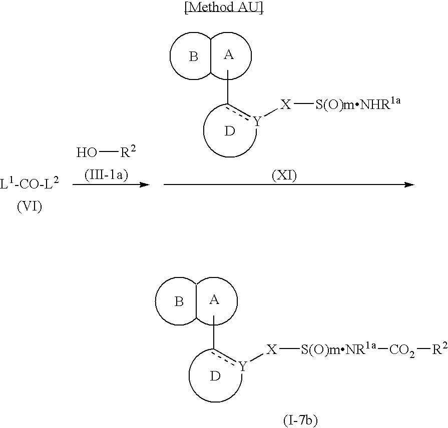

wherein the each symbol is as defined above.

In this method, compound (I-7b) can be produced from compound (XI). This reaction is carried out according to a method known per se, for example, by reacting compound (VI) with compound (III-1a) in a solvent that does not adversely influence the reaction, at −10° C. to 100° C. for 0.5 to 10 hr, and reacting the obtained compound with compound (XI) in a solvent that does not adversely influence the reaction, at −10° C. to 100° C. for 0.5 to 50 hr. This reaction may be carried out in the presence of 1 to 20 mol of a base, per 1 mol of compound (XI), where necessary.

As the base, for example, amines such as triethylamine, N,N-diisopropylethylamine, N-methylmorpholine, N,N-dimethylaniline, 1,8-diazabicyclo[5.4.0]undec-7-ene, pyridine, 4-dimethylaminopyridine and the like; alkali metal salts such as sodium hydrogencarbonate, sodium carbonate, potassium carbonate and the like, and the like can be mentioned. These bases may be used in a mixture at an appropriate ratio.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, pyridine, water and the like can be mentioned. These solvents may be used in a mixture at an appropriate ratio.

The amount of compound (VI) to be used is generally 1 to 10 mol, preferably 1 to 5 mol, per 1 mol of compound (XI).

The amount of compound (III-1a) to be used is generally 1 to 10 mol, preferably 1 to 5 mol, per 1 mol of compound (XI).

Compound (III-1a) can be produced according to a method known per se.

Compound (XV-1n), which is compound (XV) having a C₆₋₁₄ aryl group, an aromatic heterocyclic group or a C₃₋₁₀ cycloalkyl group on ring B, is produced, for example, according to the following Method AV.

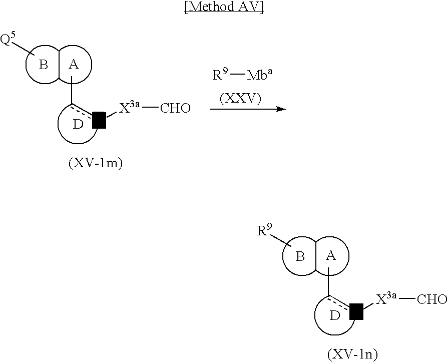

wherein the each symbol is as defined above.

In this method, compound (XV-1n) can be produced by subjecting compound (XV-1m) and compound (XXV) to a coupling reaction using an organic metal catalyst. This reaction is carried out in the same manner as in the reaction described in the aforementioned Method Z2.

Compound (XV-1m) can be produced, for example, according to the aforementioned Method Z1 to Method Z3 or a method analogous thereto.

The alkali metal salt (I-6c) of compound (I-6a), which is compound (I) wherein W is —OCONR^{1a}S(O)ₘNR^{1c}R² wherein R^{1a} is a hydrogen atom, m is 2 and the other symbols are as defined above, is produced, for example, according to the following Method AW.

### [Method AW]

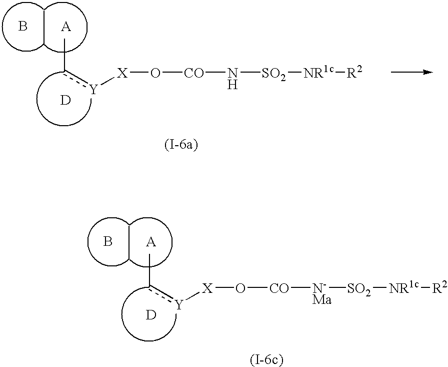

wherein the each symbol is as defined above.

As the alkali metal for Ma, sodium, potassium and the like can be mentioned.

In this method, compound (I-6c) can be produced by reacting compound (I-6a) with a base. This reaction is carried out in the same manner as in the reaction described in the aforementioned Method A2.

Compound (I-6a) can be produced, for example, according to the above-mentioned Method I or a method analogous thereto.

In each of the aforementioned reactions, when the starting material compound has an amino group, a carboxyl group, a hydroxy group or a carbonyl group as a substituent, a protecting group generally used in the peptide chemistry and the like may be introduced into these groups, and the object compound can be obtained by eliminating the protecting group as necessary after the reaction.

Examples of the amino-protecting group include a formyl group; a C₁₋₆ alkyl-carbonyl group, a C₁₋₆ alkoxy-carbonyl group, a benzoyl group, a C₇₋₁₃ aralkyl-carbonyl group (e.g., benzylcarbonyl), a C₇₋₁₃ aralkyloxy-carbonyl group (e.g., benzyloxycarbonyl, 9-fluorenylmethoxycarbonyl), a trityl group, a phthaloyl group, an N,N-dimethylaminomethylene group, a tri-substituted silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like. These groups are optionally substituted by 1 to 3 substituents selected from a halogen atom, a C₁₋₆ alkoxy group, a nitro group and the like.

Examples of the carboxyl-protecting group include a C₁₋₆ alkyl group, a C₇₋₂₀ aralkyl group (e.g., benzyl), a phenyl group, a trityl, a tri-substituted silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like. These groups are optionally substituted by 1 to 3 substituents selected from a halogen atom, a C₁₋₆ alkoxy group, a nitro group and the like.

Examples of the hydroxy-protecting group include a C₁₋₆ alkyl group, a phenyl group, a trityl group, a C₇₋₁₃ aralkyl group (e.g., benzyl), a formyl group, a C₁₋₆ alkyl-carbonyl group, a benzoyl group, a C₇₋₁₃ aralkyl-carbonyl group (e.g., benzylcarbonyl), a 2-tetrahydropyranyl group, a 2-tetrahydrofuranyl group, a tri-substituted silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like. These groups are optionally substituted by 1 to 3 substituents selected from a halogen atom, a C₁₋₆ alkyl group, a C₁₋₆ alkoxy group, a nitro group and the like.

Examples of the carbonyl-protecting group include a cyclic acetal (e.g., 1,3-dioxane), a non-cyclic acetal (e.g., a di-C₁₋₆ alkylacetal) and the like.

For elimination of the above-mentioned protecting group, a method known per se, for example, a method described in Protective Groups in Organic Synthesis, John Wiley and Sons (1980) and the like can be mentioned. For example, employed is a method using acid, base, UV light, hydrazine, phenyl hydrazine, sodium N-methyldithiocarbamate, tetrabutylammonium fluoride, palladium acetate, trialkylsilyl halide (e.g., trimethylsilyl iodide, trimethylsilyl bromide and the like) and the like, reduction and the like.

The compound of the present invention obtained by each production method mentioned above can be isolated and purified by a known means such as concentration, concentration under reduced pressure, solvent extraction, crystallization, recrystallization, phase transfer, chromatography and the like. Each starting material compound used in each of the above-mentioned production methods can be isolated and purified by a known means similar to those mentioned above. It is also possible to use such starting material compound as it is in a reaction mixture without isolation, as a starting material for the next step.

When compound (I) contains an optical isomer, a stereoisomer, a positional isomer or a rotational isomer, they are also encompassed in compound (I) and can be obtained as single products by synthesis techniques and separation techniques known per se. For example, when compound (I) contains an optical isomer, an optical isomer separated from the compound is also encompassed in compound (I).

The present invention is explained in detail in the following by referring to Experimental Example, Reference Examples, Examples and Formulation Examples, which are not to be construed as limitative.

### EXAMPLES

In the following Reference Examples and Examples, “%” means wt % unless otherwise specified, and “room temperature” means a temperature of 1° C. to 30° C. unless otherwise specified.

### Reference Example 1

### 5-(1H-indol-1-yl)-1,3-dimethyl-1H-pyrazole-4-carbaldehyde

To a solution of 1H-indole (719 mg) in N,N-dimethylformamide (10 mL), which was cooled at 0° C. in an ice bath, was added 60% sodium hydride (in oil, 275 mg) with stirring, and the mixture was stirred at 0° C. for 30 min. 5-Chloro-1,3-dimethyl-1H-pyrazole-4-carbaldehyde (900 mg) was added to this reaction mixture at 0° C., and the reaction mixture was stirred at 60° C. for 5 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 80:20, v/v) to give the title compound (1.10 g, yield 81%) as a colorless oil.

¹H-NMR (300 MHz, CDCl₃) δ:2.56 (s, 3H), 3.58 (s, 3H), 6.81 (d, J=3.0 Hz, 1H), 7.10 (d, J=7.6 Hz, 1H), 7.19 (d, J=3.4 Hz, 1H), 7.20-7.31 (m, 2H), 7.70-7.73 (m, 1H), 9.52 (s, 1H).

### Reference Example 2

### (2E)-3-[5-(1H-indol-1-yl)-1,3-dimethyl-1H-pyrazol-4-yl]acrylic acid

To a solution of 5-(1H-indol-1-yl)-1,3-dimethyl-1H-pyrazole-4-carbaldehyde obtained in Reference Example 1 (1.09 g) in acetic acid (10 mL) were added malonic acid (573 mg) and pyrrolidine (495 mg), and the mixture was stirred with heating at 100° C. for 5 hr. Malonic acid (239 mg) and pyrrolidine (648 mg) were added again to the reaction mixture, and the mixture was stirred with heating at 100° C. for 15 hr. After the reaction mixture was allowed to cool to room temperature, 1N hydrochloric acid (1 mL) and water (20 mL) were added, and the mixture was stirred at room temperature for 30 min. The resulting crystals were collected by filtration, and dissolved in ethyl acetate. The obtained solution was dried over anhydrous magnesium sulfate, and filtrated. The filtrate was concentrated, and the residue was crystallized from hexane-ethyl acetate to give the title compound (1.11 g, yield 87%) as colorless crystals.

¹H-NMR (300 MHz, DMSO-d₆) δ:2.38 (s, 3H), 3.47 (s, 3H), 5.40 (d, J=16.2 Hz, 1H), 6.86 (dd, J=3.3, 0.8 Hz, 1H), 6.97-7.11 (m, 2H), 7.15-7.27 (m, 2H), 7.57 (d, J=3.3 Hz, 1H), 7.71-7.77 (m, 1H), 12.13 (s, 1H).

By a method similar to that in Reference Example 1,5-(1H-indazol-1-yl)-1,3-dimethyl-1H-pyrazole-4-carbaldehyde of Reference Example 3, which is less polar compound, and 5-(2H-indazol-2-yl)-1,3-dimethyl-1H-pyrazole-4-carbaldehyde of Reference Example 4, which is more polar compound were obtained from 1H-indazole and 5-chloro-1,3-dimethyl-1H-pyrazole-4-carbaldehyde.

### Reference Example 3

### 5-(1H-indazol-1-yl)-1,3-dimethyl-1H-pyrazole-4-carbaldehyde

¹H-NMR (300 MHz, CDCl₃) δ:2.58 (s, 3H), 3.70 (s, 3H), 7.28-7.38 (m, 2H), 7.47-7.55 (m, 1H), 7.84-7.87 (m, 1H), 8.35 (d, J=0.9 Hz, 1H), 9.58 (s, 1H).

### Reference Example 4

### 5-(2H-indazol-2-yl)-1,3-dimethyl-1H-pyrazole-4-carbaldehyde

¹H-NMR (300 MHz, CDCl₃) δ:2.56 (s, 3H), 3.86 (s, 3H), 7.17-7.22 (m, 1H), 7.39-7.44 (m, 1H), 7.73-7.79 (m, 2H), 8.35 (d, J=0.9 Hz, 1H), 9.74 (s, 1H).

### Reference Example 5

### (2E)-3-[5-(1H-indazol-1-yl)-1,3-dimethyl-1H-pyrazol-4-yl]acrylic acid

By a method similar to that in Reference Example 2, the title compound was obtained from 5-(1H-indazol-1-yl)-1,3-dimethyl-1H-pyrazole-4-carbaldehyde obtained in Reference Example 3 and malonic acid.

¹H-NMR (300 MHz, DMSO-d₆) δ:2.40 (s, 3H), 3.51 (s, 3H), 5.46 (d, J=16.2 Hz, 1H), 7.08 (d, J=16.2 Hz, 1H), 7.27-7.37 (m, 2H), 7.49-7.55 (m, 1H), 7.96-7.99 (m, 1H), 8.60 (d, J=1.1 Hz, 1H), 12.16 (s, 1H).

### Reference Example 6

### (2E)-3-[5-(2H-indazol-2-yl)-1,3-dimethyl-1H-pyrazol-4-yl]acrylic acid

By a method similar to that in Reference Example 2, the title compound was obtained from 5-(2H-indazol-2-yl)-1,3-dimethyl-1H-pyrazole-4-carbaldehyde obtained in Reference Example 4 and malonic acid.

¹H-NMR (300 MHz, DMSO-d₆) δ:2.39 (s, 3H), 3.60 (s, 3H), 5.67 (d, J=16.2 Hz, 1H), 7.16 (d, J=16.2 Hz, 1H), 7.18-7.24 (m, 1H), 7.37-7.44 (m, 1H), 7.77 (dd, J=8.9, 0.9 Hz, 1H), 7.84-7.86 (m, 1H), 8.85 (d, J=0.9 Hz, 1H), 12.26 (s, 1H).

### Reference Example 7

### 5-(1H-benzimidazol-1-yl)-1,3-dimethyl-1H-pyrazole-4-carbaldehyde

By a method similar to that in Reference Example 1, the title compound was obtained from 1H-benzimidazole and 5-chloro-1,3-dimethyl-1H-pyrazole-4-carbaldehyde.

¹H-NMR (300 MHz, CDCl₃) δ:2.58 (s, 3H), 3.64 (s, 3H), 7.19-7.21 (m, 1H), 7.33-7.45 (m, 2H), 7.92-7.96 (m, 1H), 8.03 (s, 1H), 9.60 (s, 1H).

### Reference Example 8

### (2E)-3-[5-(1H-benzimidazol-1-yl)-1,3-dimethyl-1H-pyrazol-4-yl]acrylic acid

By a method similar to that in Reference Example 2, the title compound was obtained from 5-(1H-benzimidazol-1-yl)-1,3-dimethyl-1H-pyrazole-4-carbaldehyde obtained in Reference Example 7 and malonic acid.

¹H-NMR (300 MHz, DMSO-d₆) δ:2.40 (s, 3H), 3.54 (s, 3H), 5.49 (d, J=16.3 Hz, 1H), 7.04 (d, J=16.3 Hz, 1H), 7.22-7.28 (m, 1H), 7.32-7.45 (m, 2H), 7.84-7.88 (m, 1H), 8.55 (s, 1H), 12.20 (s, 1H).

### Reference Example 9

### 5-(1-benzothien-3-yl)-1,3-dimethyl-1H-pyrazole-4-carbaldehyde

To a mixture of 1-benzothien-3-ylboronic acid (1.61 g), 5-chloro-1,3-dimethyl-1H-pyrazole-4-carbaldehyde (1.20 g), a 2.0M aqueous sodium carbonate solution (8.0 mL) and 1,2-dimethoxyethane (25 mL) was added tetrakis(triphenylphosphine)palladium(0) (0.44 g), and the reaction mixture was heated under reflux for 6 hr under nitrogen atmosphere. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30-50:50, v/v) to give the title compound (0.83 g, yield 42%) as a brown oil.

¹H-NMR (300 MHz, CDCl₃) δ:2.58 (s, 3H), 3.68 (s, 3H), 7.38-7.51 (m, 3H), 7.62 (s, 1H), 7.97 (dd, J=6.4, 3.0 Hz, 1H), 9.57 (s, 1H).

### Reference Example 10

### (2E)-3-[5-(1-benzothien-3-yl)-1,3-dimethyl-1H-pyrazol-4-yl]acrylic acid

By a method similar to that in Reference Example 2, the title compound was obtained from 5-(1-benzothien-3-yl)-1,3-dimethyl-1H-pyrazole-4-carbaldehyde obtained in Reference Example 9 and malonic acid.

¹H-NMR (300 MHz, DMSO-d₆) δ:2.39 (s, 3H), 3.58 (s, 3H), 5.75 (d, J=16.3 Hz, 1H), 7.17 (d, J=16.3 Hz, 1H), 7.36-7.51 (m, 3H), 8.11 (s, 1H), 8.16 (d, J=7.2 Hz, 1H), 12.00 (s, 1H).

### Reference Example 11

### 1,3-dimethyl-5-(1H-pyrrolo[2,3-b]pyridin-1-yl)-1H-pyrazole-4-carbaldehyde

To a solution of 1H-pyrrolo[2,3-b]pyridine (5.60 g) in N,N-dimethylformamide (100 mL), which was cooled at 0° C. in an ice bath, was added 60% sodium hydride (in oil, 2.00 g) with stirring, and the mixture was stirred at 0° C. for 1 hr. 5-Chloro-1,3-dimethyl-1H-pyrazole-4-carbaldehyde (5.00 g) was added to this reaction mixture at 0° C., and the reaction mixture was stirred at 60° C. for 7 hr. After the reaction mixture was allowed to cool to room temperature, water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 60:40, v/v) to give the title compound (4.02 g, yield 53%) as colorless crystals.

¹H-NMR (300 MHz, CDCl₃) δ:2.55 (s, 3H), 3.68 (s, 3H), 6.78 (d, J=3.6 Hz, 1H), 7.23 (dd, J=7.9, 4.7 Hz, 1H), 7.32 (d, J=3.6 Hz, 1H), 8.03 (dd, J=7.9, 1.6 Hz, 1H), 8.36 (dd, J=4.7, 1.6 Hz, 1H), 9.58 (s, 1H).

### Reference Example 12

### ethyl (2E)-3-[1,3-dimethyl-5-(1H-pyrrolo[2,3-b]pyridin-1-yl)-1H-pyrazol-4-yl]acrylate

To a solution of ethyl (diethoxyphosphoryl)acetate (845 mg) in tetrahydrofuran (15 mL), which was cooled at 0° C. in an ice bath, was added 60% sodium hydride (in oil, 163 mg) with stirring, and the mixture was stirred at 0° C. for 15 min. A solution of 1,3-dimethyl-5-(1H-pyrrolo[2,3-b]pyridin-1-yl)-1H-pyrazole-4-carbaldehyde obtained in Reference Example 11 (780 mg) in tetrahydrofuran (8 mL) was added to this reaction mixture at 0° C., and the reaction mixture was stirred at 0° C. for 4 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 65:35, v/v) to give the title compound (929 mg, yield 92%) as colorless crystals.

¹H-NMR (300 MHz, CDCl₃) δ:1.22 (t, J=7.1 Hz, 3H), 2.45 (s, 3H), 3.58 (s, 3H), 4.13 (q, J=7.1 Hz, 2H), 5.70 (d, J=16.3 Hz, 1H), 6.77 (d, J=3.6 Hz, 1H), 7.18-7.23 (m, 2H), 7.30 (d, J=16.3 Hz, 1H), 8.03 (dd, J=7.9, 1.5 Hz, 1H), 8.35 (dd, J=4.9, 1.5 Hz, 1H).

### Reference Example 13

### (2E)-3-[1,3-dimethyl-5-(1H-pyrrolo[2,3-b]pyridin-1-yl)-1H-pyrazol-4-yl]acrylic acid

To a solution of ethyl (2E)-3-[1,3-dimethyl-5-(1H-pyrrolo[2,3-b]pyridin-1-yl)-1H-pyrazol-4-yl]acrylate obtained in Reference Example 12 (925 mg) in a mixed solvent of tetrahydrofuran (6 mL) and ethanol (6 mL) was added a 1N aqueous sodium hydroxide solution (6 mL), and the mixture was stirred with heating at 60° C. for 3 hr. The reaction mixture was allowed to cool to room temperature, neutralized with an aqueous solution (30 mL) of potassium hydrogensulfate (820 mg), and extracted with ethyl acetate. The organic layer was washed with saturated brine, dried over anhydrous magnesium sulfate, and filtrated. The filtrate was concentrated, and the residue was crystallized from hexane-ethanol to give the title compound (763 mg, yield 90%) as colorless crystals.

¹H-NMR (300 MHz, DMSO-d₆) δ:2.36 (s, 3H), 3.49 (s, 3H), 5.48 (d, J=16.2 Hz, 1H), 6.88 (d, J=3.6 Hz, 1H), 7.05 (d, J=16.2 Hz, 1H), 7.27 (dd, J=8.0, 4.9 Hz, 1H), 7.70 (d, J=3.6 Hz, 1H), 8.16 (dd, J=8.0, 1.5 Hz, 1H), 8.27 (dd, J=4.9, 1.5 Hz, 1H), 12.15 (s, 1H).

### Reference Example 14

### 1,3-dimethyl-5-(1-naphthyl)-1H-pyrazole-4-carbaldehyde

By a method similar to that in Reference Example 9, the title compound was obtained from 1-naphthylboronic acid and 5-chloro-1,3-dimethyl-1H-pyrazole-4-carbaldehyde.

¹H-NMR (300 MHz, CDCl₃) δ:2.61 (s, 3H), 3.55 (s, 3H), 7.42-7.63 (m, 5H), 7.97 (d, J=7.2 Hz, 1H), 8.04 (d, J=8.3 Hz, 1H), 9.43 (s, 1H).

### Reference Example 15

### (2E)-3-[1,3-dimethyl-5-(1-naphthyl)-1H-pyrazol-4-yl]acrylic acid

By a method similar to that in Reference Example 2, the title compound was obtained from 1,3-dimethyl-5-(1-naphthyl)-1H-pyrazole-4-carbaldehyde obtained in Reference Example 14 and malonic acid.

¹H-NMR (300 MHz, DMSO-d₆) δ:2.41 (s, 3H), 3.45 (s, 3H), 5.61 (d, J=16.2 Hz, 1H), 7.07 (d, J=16.2 Hz, 1H), 7.31 (d, J=8.3 Hz, 1H), 7.53-7.73 (m, 4H), 8.07-8.10 (m, 1H), 8.17 (d, J=8.3 Hz, 1H), 11.95 (s, 1H).

### Reference Example 16

### 1,3-dimethyl-5-(4-methyl-1H-indol-1-yl)-1H-pyrazole-4-carbaldehyde

By a method similar to that in Reference Example 1, the title compound was obtained from 4-methyl-1H-indole and 5-chloro-1,3-dimethyl-1H-pyrazole-4-carbaldehyde.

¹H-NMR (300 MHz, CDCl₃) δ:2.56 (s, 3H), 2.61 (s, 3H), 3.58 (s, 3H), 6.83 (dd, J=3.4, 0.9 Hz, 1H), 6.94 (d, J=8.3 Hz, 1H), 7.05-7.07 (m, 1H), 7.16-7.19 (m, 2H), 9.51 (s, 1H).

### Reference Example 17

### (2E)-3-[1,3-dimethyl-5-(4-methyl-1H-indol-1-yl)-1H-pyrazol-4-yl]acrylic acid

By a method similar to that in Reference Example 2, the title compound was obtained from 1,3-dimethyl-5-(4-methyl-1H-indol-1-yl)-1H-pyrazole-4-carbaldehyde obtained in Reference Example 16 and malonic acid.

¹H-NMR (300 MHz, DMSO-d₆) δ:2.37 (s, 3H), 2.55 (s, 3H), 3.46 (s, 3H), 5.45 (d, J=16.2 Hz, 1H), 6.82 (d, J=8.1 Hz, 1H), 6.89 (dd, J=3.4, 0.9 Hz, 1H), 6.98-7.01 (m, 1H), 7.06 (d, J=16.2 Hz, 1H), 7.11-7.14 (m, 1H), 7.54 (d, J=3.4 Hz, 1H), 12.13 (s, 1H).

### Reference Example 18

### 5-(4-chloro-1H-indol-1-yl)-1,3-dimethyl-1H-pyrazole-4-carbaldehyde

By a method similar to that in Reference Example 1, the title compound was obtained from 4-chloro-1H-indole and 5-chloro-1,3-dimethyl-1H-pyrazole-4-carbaldehyde.

¹H-NMR (300 MHz, CDCl₃) δ:2.56 (s, 3H), 3.58 (s, 3H), 6.94 (dd, J=3.4, 0.9 Hz, 1H), 6.97-7.04 (m, 1H), 7.15-7.30 (m, 3H), 9.53 (s, 1H).

### Reference Example 19

### (2E)-3-[5-(4-chloro-1H-indol-1-yl)-1,3-dimethyl-1H-pyrazol-4-yl]acrylic acid

By a method similar to that in Reference Example 2, the title compound was obtained from 5-(4-chloro-1H-indol-1-yl)-1,3-dimethyl-1H-pyrazole-4-carbaldehyde obtained in Reference Example 18 and malonic acid.

¹H-NMR (300 MHz, DMSO-d₆) δ:2.38 (s, 3H), 3.48 (s, 3H), 5.39 (d, J=15.9 Hz, 1H), 6.89 (d, J=3.2 Hz, 1H), 6.99-7.09 (m, 2 H), 7.20-7.25 (m, 1H), 7.27-7.31 (m, 1H), 7.72 (d, J=3.2 Hz, 1H), 12.15 (s, 1H).

### Reference Example 20

### 5-(5-fluoro-1H-indol-1-yl)-1,3-dimethyl-1H-pyrazole-4-carbaldehyde

By a method similar to that in Reference Example 1, the title compound was obtained from 5-fluoro-1H-indole and 5-chloro-1,3-dimethyl-1H-pyrazole-4-carbaldehyde.

¹H-NMR (300 MHz, CDCl₃) δ:2.55 (s, 3H), 3.58 (s, 3H), 6.77 (d, J=3.3 Hz, 1H), 7.00-7.03 (m, 2H), 7.22 (d, J=3.3 Hz, 1H), 7.33-7.37 (m, 1H), 9.51 (s, 1H).

### Reference Example 21

### (2E)-3-[5-(5-fluoro-1H-indol-1-yl)-1,3-dimethyl-1H-pyrazol-4-yl]acrylic acid

By a method similar to that in Reference Example 2, the title compound was obtained from 5-(5-fluoro-1H-indol-1-yl)-1,3-dimethyl-1H-pyrazole-4-carbaldehyde obtained in Reference Example 20 and malonic acid.

¹H-NMR (300 MHz, DMSO-d₆) δ:2.37 (s, 3H), 3.48 (s, 3H), 5.38 (d, J=16.2 Hz, 1H), 6.85 (d, J=3.4 Hz, 1H), 6.99-7.10 (m, 3H), 7.49-7.53 (m, 1H), 7.66 (d, J=3.4 Hz, 1H), 12.15 (s, 1H).

### Reference Example 22

### 5-(5-methoxy-1H-indol-1-yl)-1,3-dimethyl-1H-pyrazole-4-carbaldehyde

By a method similar to that in Reference Example 1, the title compound was obtained from 5-methoxy-1H-indole and 5-chloro-1,3-dimethyl-1H-pyrazole-4-carbaldehyde.

¹H-NMR (300 MHz, CDCl₃) δ:2.55 (s, 3H), 3.58 (s, 3H), 3.87 (s, 3H), 6.73 (dd, J=3.3, 0.9 Hz, 1H), 6.90 (dd, J=8.5, 2.4 Hz, 1H), 6.99 (dd, J=8.5, 1.5 Hz, 1H), 7.14-7.15 (m, 2H), 9.51 (s, 1H).

### Reference Example 23

### (2E)-3-[5-(5-methoxy-1H-indol-1-yl)-1,3-dimethyl-1H-pyrazol-4-yl]acrylic acid

By a method similar to that in Reference Example 2, the title compound was obtained from 5-(5-methoxy-1H-indol-1-yl)-1,3-dimethyl-1H-pyrazole-4-carbaldehyde obtained in Reference Example 22 and malonic acid.

¹H-NMR (300 MHz, DMSO-d₆) δ:2.37 (s, 3H), 3.47 (s, 3H), 3.79 (s, 3H), 5.40 (d, J=16.2 Hz, 1H), 6.76 (dd, J=3.4, 0.8 Hz, 1H), 6.81-6.85 (m, 1H), 6.89-6.93 (m, 1H), 7.07 (d, J=16.2 Hz, 1H), 7.22 (d, J=2.1 Hz, 1H), 7.51 (d, J=3.4 Hz, 1H), 12.13 (s, 1H).

### Reference Example 24

### 5-(6-chloro-1H-indol-1-yl)-1,3-dimethyl-1H-pyrazole-4-carbaldehyde

By a method similar to that in Reference Example 1, the title compound was obtained from 6-chloro-1H-indole and 5-chloro-1,3-dimethyl-1H-pyrazole-4-carbaldehyde.

¹H-NMR (300 MHz, CDCl₃) δ:2.56 (s, 3H), 3.58 (s, 3H), 6.79 (dd, J=3.4, 0.8 Hz, 1H), 7.08-7.10 (m, 1H), 7.18 (d, J=3.4 Hz, 1H), 7.22 (dd, J=8.5, 1.9 Hz, 1H), 7.62 (d, J=8.5 Hz, 1H), 9.53 (s, 1H).

### Reference Example 25

### (2E)-3-[5-(6-chloro-1H-indol-1-yl)-1,3-dimethyl-1H-pyrazol-4-yl]acrylic acid

By a method similar to that in Reference Example 2, the title compound was obtained from 5-(6-chloro-1H-indol-1-yl)-1,3-dimethyl-1H-pyrazole-4-carbaldehyde obtained in Reference Example 24 and malonic acid.

¹H-NMR (300 MHz, DMSO-d₆) δ:2.38 (s, 3H), 3.48 (s, 3H), 5.35 (d, J=16.3 Hz, 1H), 6.89 (d, J=3.4 Hz, 1H), 7.06 (d, J=16.3 Hz, 1H), 7.08-7.09 (m, 1H), 7.23 (dd, J=8.3, 1.9 Hz, 1H), 7.62 (d, J=3.4 Hz, 1H), 7.75 (d, J=8.3 Hz, 1H), 12.15 (s, 1H).

### Reference Example 26

### 5-[6-(benzyloxy)-1H-indol-1-yl]-1,3-dimethyl-1H-pyrazole-4-carbaldehyde

By a method similar to that in Reference Example 1, the title compound was obtained from 6-(benzyloxy)-1H-indole and 5-chloro-1,3-dimethyl-1H-pyrazole-4-carbaldehyde.

¹H-NMR (300 MHz, CDCl₃) δ:2.56 (s, 3H), 3.49 (s, 3H), 4.99-5.08 (m, 2H), 6.59 (d, J=2.1 Hz, 1H), 6.73 (d, J=3.4 Hz, 1H), 6.98 (dd, J=8.7, 2.1 Hz, 1H), 7.07 (d, J=3.4 Hz, 1H), 7.29-7.48 (m, 5H), 7.58 (d, J=8.7 Hz, 1H), 9.52 (s, 1H).

### Reference Example 27

### ethyl (2E)-3-{5-[6-(benzyloxy)-1H-indol-1-yl]-1,3-dimethyl-1H-pyrazol-4-yl}acrylate

By a method similar to that in Reference Example 12, the title compound was obtained from 5-[6-(benzyloxy)-1H-indol-1-yl]-1,3-dimethyl-1H-pyrazole-4-carbaldehyde obtained in Reference Example 26 and ethyl (diethoxyphosphoryl)acetate.

¹H-NMR (300 MHz, CDCl₃) δ:1.22 (t, J=7.2 Hz, 3H), 2.46 (s, 3H), 3.43 (s, 3H), 4.12 (q, J=7.2 Hz, 2H), 5.00 (s, 2H), 5.64 (d, J=16.4 Hz, 1H), 6.50 (d, J=2.1 Hz, 1H), 6.72 (dd, J=3.2, 0.8 Hz, 1H), 6.93-6.99 (m, 2H), 7.27-7.45 (m, 6H), 7.58 (d, J=8.7 Hz, 1H).

### Reference Example 28

### (2E)-3-{5-[6-(benzyloxy)-1H-indol-1-yl]-1,3-dimethyl-1H-pyrazol-4-yl}acrylic acid

By a method similar to that in Reference Example 13, the title compound was obtained from ethyl (2E)-3-{5-[6-(benzyloxy)-1H-indol-1-yl]-1,3-dimethyl-1H-pyrazol-4-yl}acrylate obtained in Reference Example 27.

¹H-NMR (300 MHz, DMSO-d₆) δ:2.38 (s, 3H), 3.44 (s, 3H), 4.99 (d, J=11.7 Hz, 1H), 5.07 (d, J=11.7 Hz, 1H), 5.43 (d, J=16.1 Hz, 1H), 6.60 (d, J=1.9 Hz, 1H), 6.76 (d, J=3.4 Hz, 1H), 6.92 (dd, J=8.5, 1.9 Hz, 1H), 7.08 (d, J=16.1 Hz, 1H), 7.27-7.44 (m, 6H), 7.60 (d, J=8.5 Hz, 1H), 12.13 (s, 1H).

### Reference Example 29

### 1,3-dimethyl-5-(2-naphthyl)-1H-pyrazole-4-carbaldehyde

By a method similar to that in Reference Example 9, the title compound was obtained from 2-naphthylboronic acid and 5-chloro-1,3-dimethyl-1H-pyrazole-4-carbaldehyde.

¹H-NMR (300 MHz, CDCl₃) δ:2.57 (s, 3H), 3.78 (s, 3H), 7.47 (dd, J=8.5, 1.7 Hz, 1H), 7.58-7.64 (m, 2H), 7.88-7.96 (m, 3H), 8.00 (d, J=8.5 Hz, 1H), 9.67 (s, 1H).

### Reference Example 30

### (2E)-3-[1,3-dimethyl-5-(2-naphthyl)-1H-pyrazol-4-yl]acrylic acid

By a method similar to that in Reference Example 2, the title compound was obtained from 1,3-dimethyl-5-(2-naphthyl)-1H-pyrazole-4-carbaldehyde obtained in Reference Example 29 and malonic acid.

¹H-NMR (300 MHz, DMSO-d₆) δ:2.37 (s, 3H), 3.68 (s, 3H), 5.89 (d, J=16.2 Hz, 1H), 7.29 (d, J=16.2 Hz, 1H), 7.53 (dd, J=8.5, 1.7 Hz, 1H), 7.60-7.70 (m, 2H), 8.00-8.06 (m, 3H), 8.12 (d, J=8.5 Hz, 1H), 12.04 (s, 1H).

### Reference Example 31

### 1,3-dimethyl-5-(quinolin-8-yl)-1H-pyrazole-4-carbaldehyde

By a method similar to that in Reference Example 9, the title compound was obtained from (quinolin-8-yl)boronic acid and 5-chloro-1,3-dimethyl-1H-pyrazole-4-carbaldehyde.

¹H-NMR (300 MHz, CDCl₃) δ:2.60 (s, 3H), 3.60 (s, 3H), 7.46-7.76 (m, 3H), 8.04 (dd, J=8.0, 1.9 Hz, 1H), 8.28 (dd, J=8.3, 1.9 Hz, 1H), 8.96 (dd, J=4.2, 1.5 Hz, 1H), 9.51 (s, 1H).

### Reference Example 32

### ethyl (2E)-3-[1,3-dimethyl-5-(quinolin-8-yl)-1H-pyrazol-4-yl]acrylate

By a method similar to that in Reference Example 12, the title compound was obtained from 1,3-dimethyl-5-(quinolin-8-yl)-1H-pyrazole-4-carbaldehyde obtained in Reference Example 31 and ethyl (diethoxyphosphoryl)acetate.

¹H-NMR (300 MHz, CDCl₃) δ:1.22 (t, J=7.2 Hz, 3H), 2.51 (s, 3H), 3.55 (s, 3H), 4.11 (q, J=7.2 Hz, 2H), 5.91 (d, J=16.4 Hz, 1H), 7.37 (d, J=16.4 Hz, 1H), 7.48 (dd, J=8.3, 4.3 Hz, 1H), 7.62-7.74 (m, 2H), 7.96-8.05 (m, 1H), 8.26 (d, J=8.3 Hz, 1H), 8.94 (d, J=4.1 Hz, 1H).

### Reference Example 33

### (2E)-3-[1,3-dimethyl-5-(quinolin-8-yl)-1H-pyrazol-4-yl]acrylic acid

By a method similar to that in Reference Example 13, the title compound was obtained from ethyl (2E)-3-[1,3-dimethyl-5-(quinolin-8-yl)-1H-pyrazol-4-yl]acrylate obtained in Reference Example 32.

¹H-NMR (300 MHz, DMSO-d₆) δ:2.39 (s, 3H), 3.43 (s, 3H), 5.69 (d, J=16.3 Hz, 1H), 7.10 (d, J=16.3 Hz, 1H), 7.64 (dd, J=8.3, 4.2 Hz, 1H), 7.73-7.88 (m, 2H), 8.23 (dd, J=6.8, 2.7 Hz, 1H), 8.53 (dd, J=8.3, 1.7 Hz, 1H), 8.91 (dd, J=4.2, 1.7 Hz, 1H), 11.90 (s, 1H).

### Reference Example 34

### 5-(5,6-difluoro-1H-indol-1-yl)-1,3-dimethyl-1H-pyrazole-4-carbaldehyde

By a method similar to that in Reference Example 1, the title compound was obtained from 5,6-difluoro-1H-indole and 5-chloro-1,3-dimethyl-1H-pyrazole-4-carbaldehyde.

¹H-NMR (300 MHz, CDCl₃) δ:2.56 (s, 3H), 3.58 (s, 3H), 6.76 (d, J=2.3 Hz, 1H), 6.88 (dd, J=9.8, 6.8 Hz, 1H), 7.20 (d, J=3.4 Hz, 1H), 7.46 (dd, J=10.2, 7.6 Hz, 1H), 9.54 (s, 1H).

### Reference Example 35

### ethyl (2E)-3-[5-(5,6-difluoro-1H-indol-1-yl)-1,3-dimethyl-1H-pyrazol-4-yl]acrylate

By a method similar to that in Reference Example 12, the title compound was obtained from 5-(5,6-difluoro-1H-indol-1-yl)-1,3-dimethyl-1H-pyrazole-4-carbaldehyde obtained in Reference Example 34 and ethyl (diethoxyphosphoryl)acetate.

¹H-NMR (300 MHz, CDCl₃) δ:1.22 (t, J=7.2 Hz, 3H), 2.45 (s, 3H), 3.50 (s, 3H), 4.13 (q, J=7.2 Hz, 2H), 5.59 (d, J=16.3 Hz, 1H), 6.72-6.83 (m, 2H), 7.10 (d, J=3.0 Hz, 1H), 7.27 (d, J=16.3 Hz, 1H), 7.45 (dd, J=10.2, 7.6 Hz, 1H).

### Reference Example 36

### (2E)-3-[5-(5,6-difluoro-1H-indol-1-yl)-1,3-dimethyl-1H-pyrazol-4-yl]acrylic acid

By a method similar to that in Reference Example 13, the title compound was obtained from ethyl (2E)-3-[5-(5,6-difluoro-1H-indol-1-yl)-1,3-dimethyl-1H-pyrazol-4-yl]acrylate obtained in Reference Example 35.

¹H-NMR (300 MHz, DMSO-d₆) δ:2.37 (s, 3H), 3.49 (s, 3H), 5.34 (d, J=16.2 Hz, 1H), 6.87 (dd, J=3.4, 0.8 Hz, 1H), 7.06 (d, J=16.2 Hz, 1H), 7.19 (dd, J=10.7, 6.8 Hz, 1H), 7.65 (d, J=3.4 Hz, 1H), 7.76 (dd, J=10.9, 7.9 Hz, 1H), 12.18 (s, 1H).

### Reference Example 37

### 5-(5-chloro-1H-indol-1-yl)-1,3-dimethyl-1H-pyrazole-4-carbaldehyde

To a solution of 5-chloro-1H-indole (2.00 g) in N,N-dimethylformamide (10 mL) was added 60% sodium hydride (in oil, 550 mg) with stirring, and the mixture was stirred at room temperature for 1 hr. 5-Chloro-1,3-dimethyl-1H-pyrazole-4-carbaldehyde (2.00 g) was added to this reaction mixture at room temperature, and the mixture was stirred with heating at 70° C. for 2 hr. After the reaction mixture was allowed to cool to room temperature, water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 85:15-65:35, v/v), and crystallized from hexane-diisopropyl ether to give the title compound (1.67 g, yield 49%) as colorless crystals.

¹H-NMR (300 MHz, CDCl₃) δ:2.56 (s, 3H), 3.58 (s, 3H), 6.76 (dd, J=3.4, 0.8 Hz, 1H), 7.02 (d, J=8.7 Hz, 1H), 7.19-7.27 (m, 2H), 7.69 (d, J=1.5 Hz, 1H), 9.53 (s, 1H).

### Reference Example 38

### (2E)-3-[5-(5-chloro-1H-indol-1-yl)-1,3-dimethyl-1H-pyrazol-4-yl]acrylic acid

To a solution of 5-(5-chloro-1H-indol-1-yl)-1,3-dimethyl-1H-pyrazole-4-carbaldehyde obtained in Reference Example 37 (1.91 g) in pyridine (10 mL) were added malonic acid (2.90 g) and piperidine (1.7 mL), and the mixture was stirred with heating at 110° C. for 2.5 hr. After the reaction mixture was allowed to cool to room temperature, the reaction mixture was concentrated under reduced pressure, and the residue was extracted with ethyl acetate. The organic layer was washed successively with saturated aqueous ammonium chloride solution and saturated brine, dried over anhydrous magnesium sulfate, and filtrated. The filtrate was concentrated, and the residue was subjected to silica gel column chromatography (hexane-ethyl acetate 65:35-25:75, v/v) to give the title compound (1.74 g, yield 79%) as a colorless amorphous solid.

¹H-NMR (300 MHz, CDCl₃) δ:2.45 (s, 3H), 3.51 (s, 3H), 5.54 (d, J=15.9 Hz, 1H), 6.74 (d, J=3.4 Hz, 1H), 6.90 (d, J=8.7 Hz, 1H), 7.10 (d, J=3.4 Hz, 1H), 7.19 (dd, J=8.7, 2.3 Hz, 1H), 7.32 (d, J=15.9 Hz, 1H), 7.68 (d, J=1.9 Hz, 1H).

### Reference Example 39

### 5-(3-chloro-1H-indazol-1-yl)-1,3-dimethyl-1H-pyrazole-4-carbaldehyde

A mixture of 3-chloro-1H-indazole (1.53 g), 5-chloro-1,3-dimethyl-1H-pyrazole-4-carbaldehyde (1.00 g) and potassium carbonate (1.38 g) in N,N-dimethylformamide (30 mL) was stirred with heating at 120° C. for 12 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 95:5-75:25, v/v) to give the title compound (1.65 g, yield 60%) as colorless crystals.

¹H-NMR (300 MHz, CDCl₃) δ:2.56 (s, 3H), 3.73 (s, 3H), 7.28 (dd, J=7.5, 0.9 Hz, 1H), 7.30-7.41 (m, 1H), 7.54-7.58 (m, 1H), 7.81 (dd, J=7.5, 0.9 Hz, 1H), 9.60 (s, 1H).

### Reference Example 40

### (2E)-3-[5-(3-chloro-1H-indazol-1-yl)-1,3-dimethyl-1H-pyrazol-4-yl]acrylic acid

A solution of 5-(3-chloro-1H-indazol-1-yl)-1,3-dimethyl-1H-pyrazole-4-carbaldehyde obtained in Reference Example 39 (1.65 g) and ethyl (diethoxyphosphoryl)acetate (1.41 g) in tetrahydrofuran (30 mL) was cooled at 0° C. in an ice bath, 60% sodium hydride (in oil, 252 mg) was added with stirring, and the mixture was stirred at room temperature for 2 hr. Water was added to the reaction mixture, and the mixture was extracted with ethyl acetate. The organic layer was washed with saturated brine, dried over anhydrous magnesium sulfate, and filtrated. The filtrate was concentrated, and the residue was dissolved in a mixed solvent of tetrahydrofuran (30 mL) and methanol (30 mL). A 4N aqueous sodium hydroxide solution (4 mL) was added, and the mixture was stirred at room temperature for 3 hr. 1N Hydrochloric acid (20 mL) was added to the reaction mixture, and the mixture was extracted with ethyl acetate. The organic layer was washed with saturated brine, dried over anhydrous magnesium sulfate, and filtrated. The filtrate was concentrated to give the title compound (1.59 g, yield 84%) as colorless crystals.

¹H-NMR (300 MHz, DMSO-d₆) δ:2.40 (s, 3H), 3.56 (s, 3H), 5.50 (d, J=16.2 Hz, 1H), 7.06 (d, J=16.2 Hz, 1H), 7.35 (d, J=8.4 Hz, 1H), 7.46 (t, J=7.5 Hz, 1H), 7.61-7.66 (m, 1H), 7.89 (d, J=7.8 Hz, 1H), 12.21 (br s, 1H).

### Reference Example 41

### 1,3-dimethyl-5-[6-(trifluoromethyl)-1H-indol-1-yl]-1H-pyrazole-4-carbaldehyde

By a method similar to that in Reference Example 1, the title compound was obtained from 6-(trifluoromethyl)-1H-indole and 5-chloro-1,3-dimethyl-1H-pyrazole-4-carbaldehyde.

¹H-NMR (300 MHz, CDCl₃) δ:2.59 (s, 3H), 3.59 (s, 3H), 6.71-6.99 (m, 1H), 7.29-7.38 (m, 2H), 7.43-7.58 (m, 1H), 7.82 (d, J=8.3 Hz, 1H), 9.55 (s, 1H).

### Reference Example 42

### (2E)-3-{1,3-dimethyl-5-[6-(trifluoromethyl)-1H-indol-1-yl]-1H-pyrazol-4-yl}acrylic acid

By a method similar to that in Reference Example 40, the title compound was obtained from 1,3-dimethyl-5-[6-(trifluoromethyl)-1H-indol-1-yl]-1H-pyrazole-4-carbaldehyde obtained in Reference Example 41.

¹H-NMR (300 MHz, DMSO-d₆) δ:2.40 (s, 3H), 3.50 (s, 3H), 5.35 (d, J=16.3 Hz, 1H), 6.87-7.19 (m, 2H), 7.21-7.40 (m, 1H), 7.44-7.65 (m, 1H), 7.85 (d, J=3.4 Hz, 1H), 7.96 (d, J=8.3 Hz, 1H), 11.99 (s, 1H).

### Reference Example 43

### ethyl 3-[1,3-dimethyl-5-(1H-pyrrolo[2,3-b]pyridin-1-yl)-1H-pyrazol-4-yl]propanoate

Ethyl (2E)-3-[1,3-dimethyl-5-(1H-pyrrolo[2,3-b]pyridin-1-yl)-1H-pyrazol-4-yl]acrylate obtained in Reference Example 12 (7.49 g) was dissolved in ethanol (200 mL), 10% palladium carbon (800 mg) was added, and the mixture was stirred under 1 atom of hydrogen atmosphere at room temperature for 5 hr. The catalyst was removed by filtration, and the filtrate was concentrated. The residue was subjected to silica gel column chromatography (hexane-ethyl acetate 50:50-0:100, v/v) to give the title compound (6.64 g, yield 88%) as a colorless oil.

¹H-NMR (300 MHz, CDCl₃) δ:1.15 (t, J=7.2 Hz, 3H), 2.25-2.35 (m, 5H), 2.55-2.64 (m, 2H), 3.51 (s, 3H), 4.00 (q, J=7.2 Hz, 2H), 6.70 (d, J=3.6 Hz, 1H), 7.17 (dd, J=7.8, 4.8 Hz, 1H), 7.21 (d, J=3.6 Hz, 1H), 8.00 (dd, J=7.8, 1.6 Hz, 1H), 8.34 (dd, J=4.8, 1.6 Hz, 1H).

### Reference Example 44

### ethyl 3-[5-(2,3-dihydro-1H-pyrrolo[2,3-b]pyridin-1-yl)-1,3-dimethyl-1H-pyrazol-4-yl]propanoate

By a method similar to that in Reference Example 43, the title compound (more polar compound, 0.48 g, yield 6%) was obtained as a colorless oil, together with the compound of Reference Example 43, from ethyl (2E)-3-[1,3-dimethyl-5-(1H-pyrrolo[2,3-b]pyridin-1-yl)-1H-pyrazol-4-yl]acrylate obtained in Reference Example 12.

¹H-NMR (300 MHz, CDCl₃) δ:1.20 (t, J=7.1 Hz, 3H), 2.21 (s, 3H), 2.36-2.45 (m, 2H), 2.55-2.65 (m, 2H), 3.17-3.27 (m, 2H), 3.63 (s, 3H), 3.85 (t, J=8.6 Hz, 2H), 4.05 (q, J=7.1 Hz, 2H), 6.59 (dd, J=7.0, 5.3 Hz, 1H), 7.34 (dd, J=7.0, 1.0 Hz, 1H), 7.88 (dd, J=5.3, 1.0 Hz, 1H).

### Reference Example 45

### 3-[1,3-dimethyl-5-(1H-pyrrolo[2,3-b]pyridin-1-yl)-1H-pyrazol-4-yl]propanoic acid

By a method similar to that in Reference Example 13, the title compound was obtained from ethyl 3-[1,3-dimethyl-5-(1H-pyrrolo[2,3-b]pyridin-1-yl)-1H-pyrazol-4-yl]propanoate obtained in Reference Example 43.

¹H-NMR (300 MHz, DMSO-d₆) δ:2.08-2.17 (m, 2H), 2.19 (s, 3H), 2.31-2.45 (m, 2H), 3.40 (s, 3H), 6.79 (d, J=3.6 Hz, 1H), 7.23 (dd, J=7.8, 4.7 Hz, 1H), 7.65 (d, J=3.6 Hz, 1H), 8.11 (dd, J=7.8, 1.6 Hz, 1H), 8.25 (dd, J=4.7, 1.6 Hz, 1H), 12.02 (s, 1H).

### Reference Example 46

### 3-[5-(2,3-dihydro-1H-pyrrolo[2,3-b]pyridin-1-yl)-1,3-dimethyl-1H-pyrazol-4-yl]propanoic acid

By a method similar to that in Reference Example 13, the title compound was obtained from ethyl 3-[5-(2,3-dihydro-1H-pyrrolo[2,3-b]pyridin-1-yl)-1,3-dimethyl-1H-pyrazol-4-yl]propanoate obtained in Reference Example 44.

¹H-NMR (300 MHz, DMSO-d₆) δ:2.08 (s, 3H), 2.21-2.29 (m, 2H), 2.37-2.44 (m, 2H), 3.15-3.22 (m, 2H), 3.49 (s, 3H), 3.70-3.88 (m, 2H), 6.61 (dd, J=7.1, 5.2 Hz, 1H), 7.43 (dd, J=7.1, 1.5 Hz, 1H), 7.73 (dd, J=5.2, 1.5 Hz, 1H), 12.02 (s, 1H).

### Reference Example 47

### ethyl (2E)-3-[1,3-dimethyl-5-(1H-pyrrolo[2,3-b]pyridin-1-yl)-1H-pyrazol-4-yl]-2-methylacrylate

By a method similar to that in Reference Example 12, the title compound was obtained from 1,3-dimethyl-5-(1H-pyrrolo[2,3-b]pyridin-1-yl)-1H-pyrazole-4-carbaldehyde obtained in Reference Example 11 and ethyl 2-(diethoxyphosphoryl)propanoate.

¹H-NMR (300 MHz, CDCl₃) δ:1.21 (s, 3H), 1.25 (t, J=7.1 Hz, 3H), 2.29 (s, 3H), 3.66 (s, 3H), 4.15 (q, J=7.1 Hz, 2H), 6.65 (d, J=3.4 Hz, 1H), 7.10 (d, J=3.4 Hz, 1H), 7.18 (dd, J=8.0, 4.7 Hz, 1H), 7.32 (s, 1H), 7.97 (dd, J=8.0, 1.5 Hz, 1H), 8.34-8.40 (m, 1H).

### Reference Example 48

### (2E)-3-[1,3-dimethyl-5-(1H-pyrrolo[2,3-b]pyridin-1-yl)-1H-pyrazol-4-yl]-2-methylacrylic acid

By a method similar to that in Reference Example 13, the title compound was obtained from ethyl (2E)-3-[1,3-dimethyl-5-(1H-pyrrolo[2,3-b]pyridin-1-yl)-1H-pyrazol-4-yl]-2-methylacrylate obtained in Reference Example 47.

¹H-NMR (300 MHz, DMSO-d₆) δ:1.06 (d, J=1.3 Hz, 3H), 2.20 (s, 3H), 3.54 (s, 3H), 6.78 (d, J=3.8 Hz, 1H), 7.20 (d, J=1.3 Hz, 1H), 7.25 (dd, J=7.9, 4.7 Hz, 1H), 7.56 (d, J=3.8 Hz, 1H), 8.11 (dd, J=7.9, 1.5 Hz, 1H), 8.30 (dd, J=4.7, 1.5 Hz, 1H), 12.32 (s, 1H).

### Reference Example 49

### 5-chloro-N-methoxy-N,1-dimethyl-1H-pyrazole-4-carboxamide

To a mixture of N,O-dimethylhydroxyamine hydrochloride (6.78 g) and N,N-dimethylformamide (50 mL) was added triethylamine (9.68 mL), and the mixture was stirred at room temperature for 10 min. 5-Chloro-1-methyl-1H-pyrazole-4-carboxylic acid (9.70 g), N-[3-(dimethylamino)propyl]-N′-ethylcarbodiimide hydrochloride (13.32 g) and 1-hydroxybenzotriazole monohydrate (10.64 g) were added to this reaction mixture, and the mixture was stirred at room temperature for 15 hr. Water was added to the reaction mixture, and the mixture was extracted with ethyl acetate. The organic layer was washed successively with aqueous potassium carbonate solution and saturated brine, dried over anhydrous magnesium sulfate, and filtrated. The filtrate was concentrated, and the residue was subjected to silica gel column chromatography (hexane-ethyl acetate 67:33-25:75, v/v) to give the title compound (9.92 g, yield 81%) as a colorless oil.

¹H-NMR (300 MHz, CDCl₃) δ:3.33 (s, 3H), 3.66 (s, 3H), 3.88 (s, 3H), 7.92 (s, 1H).

### Reference Example 50

### 5-chloro-1-methyl-1H-pyrazole-4-carbaldehyde

To a solution of 5-chloro-N-methoxy-N,1-dimethyl-1H-pyrazole-4-carboxamide obtained in Reference Example 49 (9.47 g) in tetrahydrofuran (60 mL) was added dropwise diisobutylaluminum hydride (1.5M toluene solution, 37.2 mL) with stirring at 0° C., and the reaction mixture was stirred at 0° C. for 1 hr. Magnesium sulfate 10 hydrate (19.0 g) was gradually added, and the mixture was stirred at room temperature for 5 hr. The precipitate was removed by filtration, and the filtrate was concentrated. The residue was subjected to silica gel column chromatography (hexane-ethyl acetate 75:25-33:67, v/v), and crystallized from hexane-diethyl ether to give the title compound (2.88 g, yield 43%) as colorless crystals.

¹H-NMR (306 MHz, CDCl₃) δ:3.90 (s, 3H), 7.96 (s, 1H), 9.83 (s, 1H).

### Reference Example 51

### 1-methyl-5-(1H-pyrrolo[2,3-b]pyridin-1-yl)-1H-pyrazole-4-carbaldehyde

By a method similar to that in Reference Example 1, the title compound was obtained from 5-chloro-1-methyl-1H-pyrazole-4-carbaldehyde obtained in Reference Example 50 and 1H-pyrrolo[2,3-b]pyridine.

¹H-NMR (300 MHz, CDCl₃) δ:3.78 (s, 3H), 6.79 (d, J=3.8 Hz, 1H), 7.23 (dd, J=7.9, 4.7 Hz, 1H), 7.34 (d, J=3.8 Hz, 1H), 8.03 (dd, J=7.9, 1.6 Hz, 1H), 8.10 (s, 1H), 8.36 (dd, J=4.7, 1.6 Hz, 1H), 9.62 (s, 1H).

### Reference Example 52

### ethyl (2E)-2-methyl-3-[1-methyl-5-(1H-pyrrolo[2,3-b]pyridin-1-yl)-1H-pyrazol-4-yl]acrylate

By a method similar to that in Reference Example 12, the title compound was obtained from 1-methyl-5-(1H-pyrrolo[2,3-b]pyridin-1-yl)-1H-pyrazole-4-carbaldehyde obtained in Reference Example 51 and ethyl 2-(diethoxyphosphoryl)propanoate.

¹H-NMR (300 MHz, CDCl₃) δ:1.21 (t, J=7.1 Hz, 3H), 2.10 (d, J=1.1 Hz, 3H), 3.70 (s, 3H), 4.13 (q, J=7.1 Hz, 2H), 6.74 (d, J=3.6 Hz, 1H), 7.08 (d, J=1.1 Hz, 1H), 7.17-7.24 (m, 2H), 7.91 (s, 1H), 8.02 (dd, J=7.8, 1.6 Hz, 1H), 8.36 (dd, J=4.8, 1.6 Hz, 1H).

### Reference Example 53

### (2E)-2-methyl-3-[1-methyl-5-(1H-pyrrolo[2,3-b]pyridin-1-yl)-1H-pyrazol-4-yl]acrylic acid

By a method similar to that in Reference Example 13, the title compound was obtained from ethyl (2E)-2-methyl-3-[1-methyl-5-(1H-pyrrolo[2,3-b]pyridin-1-yl)-1H-pyrazol-4-yl]acrylate obtained in Reference Example 52.

¹H-NMR (300 MHz, DMSO-d₆) δ:2.00 (s, 3H), 3.59 (s, 3H), 6.84 (s, 1H), 6.86 (d, J=3.6 Hz, 1H), 7.28 (dd, J=8.0, 4.5 Hz, 1H), 7.73 (d, J=3.6 Hz, 1H), 8.02 (s, 1H), 8.16 (dd, J=8.0, 1.6 Hz, 1H), 8.28 (dd, J=4.5, 1.6 Hz, 1H), 12.19 (s, 1H).

### Reference Example 54

### 5-(6-hydroxy-2,3-dihydro-1H-indol-1-yl)-1,3-dimethyl-1H-pyrazole-4-carbaldehyde

5-[6-(Benzyloxy)-1H-indol-1-yl]-1,3-dimethyl-1H-pyrazole-4-carbaldehyde obtained in Reference Example 26 (1.59 g) was dissolved in methanol (120 mL), 10% palladium carbon (330 mg) was added, and the mixture was stirred under 1 atom of hydrogen atmosphere at room temperature for 24 hr. The catalyst was removed by filtration, and the filtrate was concentrated. The residue was subjected to silica gel column chromatography (hexane-ethyl acetate 60:40, v/v) to give the title compound (574 mg, yield 48%) as colorless crystals.

¹H-NMR (300 MHz, CDCl₃) δ:2.45 (s, 3H), 3.14-3.22 (m, 2H), 3.67 (s, 3H), 3.83-3.95 (m, 1H), 3.98-4.07 (m, 1H), 5.54 (br s, 1H), 5.80 (d, J=2.1 Hz, 1H), 6.28 (dd, J=8.0, 2.1 Hz, 1H), 7.01 (d, J=8.0 Hz, 1H), 9.77 (s, 1H).

### Reference Example 55

### 5-(6-methoxy-2,3-dihydro-1H-indol-1-yl)-1,3-dimethyl-1H-pyrazole-4-carbaldehyde

A mixture of 5-(6-hydroxy-2,3-dihydro-1H-indol-1-yl)-1,3-dimethyl-1H-pyrazole-4-carbaldehyde obtained in Reference Example 54 (267 mg), methyl iodide (2.61 g) and potassium carbonate (430 mg) in acetone (8 mL) was heated under reflux for 24 hr. The reaction mixture was concentrated under reduced pressure, water was added to the residue, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 75:25, v/v) to give the title compound (250 mg, yield 89%) as a colorless oil.

¹H-NMR (300 MHz, CDCl₃) δ:2.48 (s, 3H), 3.14-3.26 (m, 2H), 3.68 (s, 3H), 3.69 (s, 3H), 3.83-3.93 (m, 1H), 3.97-4.07 (m, 1H), 5.88 (d, J=2.3 Hz, 1H), 6.34 (dd, J=8.0, 2.3 Hz, 1H), 7.07 (d, J=8.0 Hz, 1H), 9.77 (s, 1H).

### Reference Example 56

### ethyl (2E)-3-[5-(6-methoxy-2,3-dihydro-1H-indol-1-yl)-1,3-dimethyl-1H-pyrazol-4-yl]acrylate

By a method similar to that in Reference Example 12, the title compound was obtained from 5-(6-methoxy-2,3-dihydro-1H-indol-1-yl)-1,3-dimethyl-1H-pyrazole-4-carbaldehyde obtained in Reference Example 55 and ethyl (diethoxyphosphoryl)acetate.

¹H-NMR (300 MHz, CDCl₃) δ:1.26 (t, J=7.2 Hz, 3H), 2.38 (s, 3H), 3.14-3.25 (m, 2H), 3.63 (s, 3H), 3.67 (s, 3H), 3.75-3.95 (m, 2H), 4.16 (q, J=7.2 Hz, 2H), 5.76 (d, J=2.3 Hz, 1H), 5.94 (d, J=16.1 Hz, 1H), 6.31 (dd, J=8.0, 2.3 Hz, 1H), 7.06 (d, J=8.0 Hz, 1H), 7.45 (d, J=16.1 Hz, 1H).

### Reference Example 57

### (2E)-3-[5-(6-methoxy-2,3-dihydro-1H-indol-1-yl)-1,3-dimethyl-1H-pyrazol-4-yl]acrylic acid

By a method similar to that in Reference Example 13, the title compound was obtained from ethyl (2E)-3-[5-(6-methoxy-2,3-dihydro-1H-indol-1-yl)-1,3-dimethyl-1H-pyrazol-4-yl]acrylate obtained in Reference Example 56.

¹H-NMR (300 MHz, DMSO-d₆) δ:2.28 (s, 3H), 3.10-3.18 (m, 2H), 3.59 (s, 3H), 3.60 (s, 3H), 3.65-3.77 (m, 1H), 3.87-3.97 (m, 1H), 5.65 (d, J=2.3 Hz, 1H), 5.77 (d, J=16.2 Hz, 1H), 6.29 (dd, J=8.1, 2.3 Hz, 1H), 7.08 (d, J=8.1 Hz, 1H), 7.25 (d, J=16.2 Hz, 1H), 12.06 (s, 1H).

### Reference Example 58

### 5-(6-methoxy-1H-indol-1-yl)-1,3-dimethyl-1H-pyrazole-4-carbaldehyde

By a method similar to that in Reference Example 1, the title compound was obtained from 6-methoxy-1H-indole and 5-chloro-1,3-dimethyl-1H-pyrazole-4-carbaldehyde.

¹H-NMR (300 MHz, CDCl₃) δ:2.57 (s, 3H), 3.59 (s, 3H), 3.78 (s, 3H), 6.53 (d, J=2.3 Hz, 1H), 6.73 (dd, J=3.4, 0.8 Hz, 1H), 6.90 (dd, J=8.6, 2.3 Hz, 1H), 7.07 (d, J=3.4 Hz, 1H), 7.57 (d, J=8.6 Hz, 1H), 9.54 (s, 1H).

### Reference Example 59

### ethyl (2E)-3-[5-(6-methoxy-1H-indol-1-yl)-1,3-dimethyl-1H-pyrazol-4-yl]acrylate

By a method similar to that in Reference Example 12, the title compound was obtained from 5-(6-methoxy-1H-indol-1-yl)-1,3-dimethyl-1H-pyrazole-4-carbaldehyde obtained in Reference Example 58 and ethyl (diethoxyphosphoryl)acetate.

¹H-NMR (300 MHz, CDCl₃) δ:1.22 (t, J=7.2 Hz, 3H), 2.47 (s, 3H), 3.51 (s, 3H), 3.76 (s, 3H), 4.12 (q, J=7.2 Hz, 2H), 5.65 (d, J=16.3 Hz, 1H), 6.42 (d, J=2.1 Hz, 1H), 6.72 (d, J=3.2 Hz, 1H), 6.88 (dd, J=8.7, 2.1 Hz, 1H), 6.96 (d, J=3.2 Hz, 1H), 7.32 (d, J=16.3 Hz, 1H), 7.57 (d, J=8.7 Hz, 1H).

### Reference Example 60

### (2E)-3-[5-(6-methoxy-1H-indol-1-yl)-1,3-dimethyl-1H-pyrazol-4-yl]acrylic acid

By a method similar to that in Reference Example 13, the title compound was obtained from ethyl (2E)-3-[5-(6-methoxy-1H-indol-1-yl)-1,3-dimethyl-1H-pyrazol-4-yl]acrylate obtained in Reference Example 59.

¹H-NMR (300 MHz, DMSO-d₆) δ:2.38 (s, 3H), 3.49 (s, 3H), 3.69 (s, 3H), 5.43 (d, J=16.2 Hz, 1H), 6.48 (d, J=2.1 Hz, 1H), 6.76 (dd, J=3.4, 0.8 Hz, 1H), 6.84 (dd, J=8.7, 2.1 Hz, 1H), 7.10 (d, J=16.2 Hz, 1H), 7.38 (d, J=3.4 Hz, 1H), 7.59 (d, J=8.7 Hz, 1H), 12.14 (s, 1H).

### Reference Example 61

### [5-(5-chloro-1H-indol-1-yl)-1,3-dimethyl-1H-pyrazol-4-yl]methanol

5-(5-Chloro-1H-indol-1-yl)-1,3-dimethyl-1H-pyrazole-4-carbaldehyde obtained in Reference Example 37 (4.08 g) was dissolved in a mixed solvent of tetrahydrofuran (24 mL) and methanol (6 mL), sodium borohydride (845 mg) was added, and the mixture was stirred at 0° C. for 3 hr. Water was added to this reaction mixture, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (3.99 g, yield 97%) as colorless crystals.

¹H-NMR (300 MHz, CDCl₃) δ:1.28 (t, J=5.2 Hz, 1H), 2.38 (s, 3H), 3.50 (s, 3H), 4.22-4.39 (m, 2H), 6.68 (dd, J=3.3, 0.8 Hz, 1H), 6.99 (d, J=8.7 Hz, 1H), 7.15-7.22 (m, 2H), 7.66 (d, J=1.5 Hz, 1H).

### Reference Example 62

### [5-(5-chloro-1H-indol-1-yl)-1,3-dimethyl-1H-pyrazol-4-yl]acetaldehyde

5-(5-Chloro-1H-indol-1-yl)-1,3-dimethyl-1H-pyrazole-4-carbaldehyde obtained in Reference Example 37 (5.26 g) was dissolved in a mixed solvent of tetrahydrofuran (30 mL) and tert-butyl alcohol (30 mL), potassium tert-butoxide (2.59 g) and ethyl bromoacetate (3.53 g) were added, and the mixture was stirred at room temperature for 5 hr. This reaction mixture was filtrated through Celite®, and the filtrate was concentrated. The residue was dissolved in a mixed solvent of tetrahydrofuran (10 mL) and ethanol (10 mL), a 8N aqueous sodium hydroxide solution (5 ml) was added, and the mixture was stirred at room temperature for 3 hr. Acetic acid (30 ml) was added, and the mixture was stirred at 60° C. for 3 hr. Water was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 70:30, v/v) to give the title compound (2.89 g, yield 52%) as a pale-yellow oil.

¹H-NMR (300 MHz, CDCl₃) δ:2.24 (s, 3H), 3.20-3.40 (m, 2H), 3.50 (s, 3H), 6.67 (dd, J=3.3, 0.8 Hz, 1H), 6.94 (d, J=8.7 Hz, 1H), 7.09 (d, J=3.4 Hz, 1H), 7.19 (dd, J=8.7, 1.9 Hz, 1H), 7.65 (d, J=1.5 Hz, 1H), 9.51 (t, J=1.5 Hz, 1H).

### Reference Example 63

### 2-[5-(5-chloro-1H-indol-1-yl)-1,3-dimethyl-1H-pyrazol-4-yl]ethanol

By a method similar to that in Reference Example 61, the title compound was obtained from [5-(5-chloro-1H-indol-1-yl)-1,3-dimethyl-1H-pyrazol-4-yl]acetaldehyde obtained in Reference Example 62.

¹H-NMR (300 MHz, CDCl₃) δ:2.31 (s, 3H), 2.37-2.57 (m, 2H), 3.44-3.55 (m, 5H), 6.66 (dd, J=3.2, 0.8 Hz, 1H), 6.94 (d, J=8.7 Hz, 1H), 7.13 (d, J=3.2 Hz, 1H), 7.18 (dd, J=8.7, 2.1 Hz, 1H), 7.66 (d, J=1.9 Hz, 1H).

### Reference Example 64

### ethyl (2E)-3-[5-(5-chloro-1H-indol-1-yl)-1,3-dimethyl-1H-pyrazol-4-yl]acrylate

By a method similar to that in Reference Example 12, the title compound was obtained from 5-(5-chloro-1H-indol-1-yl)-1,3-dimethyl-1H-pyrazole-4-carbaldehyde obtained in Reference Example 37 and ethyl (diethoxyphosphoryl)acetate.

¹H-NMR (300 MHz, CDCl₃) δ:1.22 (t, J=7.2 Hz, 3H), 2.46 (s, 3H), 3.49 (s, 3H), 4.12 (q, J=6.9 Hz, 2H), 5.60 (d, J=16.3 Hz, 1H), 6.75 (d, J=3.4 Hz, 1H), 6.92 (d, J=8.7 Hz, 1H), 7.11 (d, J=3.4 Hz, 1H), 7.20 (dd, J=8.7, 1.9 Hz, 1H), 7.28 (d, J=16.3 Hz, 1H), 7.69 (d, J=1.9 Hz, 1H)

### Reference Example 65

### ethyl 3-[5-(5-chloro-1H-indol-1-yl)-1,3-dimethyl-1H-pyrazol-4-yl]propanoate

Ethyl (2E)-3-[5-(5-chloro-1H-indol-1-yl)-1,3-dimethyl-1H-pyrazol-4-yl]acrylate obtained in Reference Example 64 (5.09 g) was dissolved in a mixed solvent of tetrahydrofuran (25 mL) and ethanol (25 mL), platinum oxide (500 mg) was added, and the mixture was stirred under 1 atom of hydrogen atmosphere at room temperature for 16 hr. The catalyst was removed by filtration, and the filtrate was concentrated. The residue was subjected to silica gel column chromatography (hexane-ethyl acetate 75:25, v/v) to give the title compound (4.91 g, yield 96%) as a colorless oil.

¹H-NMR (300 MHz, CDCl₃) δ:1.12-1.19 (m, 3H), 2.18-2.25 (m, 2H), 2.30 (s, 3H), 2.47-2.66 (m, 2H), 3.41-3.45 (m, 3H), 3.94-4.03 (m, 2H), 6.66-6.70 (m, 1H), 6.92 (d, J=8.7 Hz, 1H), 7.11 (d, J=3.4 Hz, 1H), 7.19 (dd, J=8.7, 2.1 Hz, 1H), 7.66 (d, J=1.9 Hz, 1H).

### Reference Example 66

### 3-[5-(5-chloro-1H-indol-1-yl)-1,3-dimethyl-1H-pyrazol-4-yl]propan-1-ol

To a solution of ethyl 3-[5-(5-chloro-1H-indol-1-yl)-1,3-dimethyl-1H-pyrazol-4-yl]propanoate obtained in Reference Example 65 (4.11 g) in tetrahydrofuran (24 mL), which was cooled at 0° C. in an ice bath, was added diisobutylaluminum hydride (1.5M toluene solution, 20 mL) by small portions with stirring. The reaction mixture was stirred at room temperature for 1 hr, and was cooled again at 0° C. in an ice bath. Methanol and water were added to the reaction mixture with stirring, the mixture was filtrated through Celite®, and the filtrate was concentrated. The residue was subjected to silica gel column chromatography (hexane-ethyl acetate 50:50, v/v) to give the title compound (3.47 g, yield 96%) as a colorless oil.

¹H-NMR (300 MHz, CDCl₃) δ:1.44-1.56 (m, 2H), 2.20-2.40 (m, 5H), 3.39-3.47 (m, 5H), 6.66 (dd, J=3.4, 0.8 Hz, 1H), 6.94 (d, J=8.7 Hz, 1H), 7.10 (d, J=3.4 Hz, 1H), 7.17 (dd, J=8.7, 2.1 Hz, 1H), 7.65 (d, J=1.7 Hz, 1H).

### Reference Example 67

### [5-(5-chloro-1H-indol-1-yl)-1,3-dimethyl-1H-pyrazol-4-yl]acetic acid

[5-(5-Chloro-1H-indol-1-yl)-1,3-dimethyl-1H-pyrazol-4-yl]acetaldehyde obtained in Reference Example 62 (501 mg) was dissolved in a mixed solvent of tert-butyl alcohol (5.8 mL) and water (1.2 mL). Sodium dihydrogenphosphate (627 mg), sodium chlorite (236 mg) and 2-methyl-2-butene (611 mg) were added, and the mixture was stirred at room temperature for 14 hr. Water was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (ethyl acetate), and crystallized from hexane-ethyl acetate to give the title compound (412 mg, yield 78%) as pale-yellow crystals.

¹H-NMR (300 MHz, DMSO-d₆) δ:2.17 (s, 3H), 3.01-3.16 (m, 2H), 3.40 (s, 3H), 6.75 (d, J=3.0 Hz, 1H), 7.04 (d, J=8.7 Hz, 1H), 7.18 (dd, J=8.5, 2.1 Hz, 1H), 7.51 (d, J=3.4 Hz, 1H), 7.74 (d, J=1.9 Hz, 1H), 12.25 (br s, 1H).

### Reference Example 68

### 3-[5-(5-chloro-1H-indol-1-yl)-1,3-dimethyl-1H-pyrazol-4-yl]propanoic acid

By a method similar to that in Reference Example 65, the title compound was obtained from (2E)-3-[5-(5-chloro-1H-indol-1-yl)-1,3-dimethyl-1H-pyrazol-4-yl]acrylic acid obtained in Reference Example 38.

¹H-NMR (300 MHz, DMSO-d₆) δ:2.09 (t, J=7.8 Hz, 2H), 2.20 (s, 3 H), 2.49-2.53 (m, 2H), 3.37 (s, 3H), 6.76 (d, J=2.7 Hz, 1H), 7.02 (d, J=8.7 Hz, 1H), 7.20 (dd, J=8.7, 1.9 Hz, 1H), 7.56 (d, J=3.0 Hz, 1H), 7.75 (d, J=1.9 Hz, 1H), 12.07 (br s, 1H).

### Reference Example 69

### ethyl (2E)-4-[5-(5-chloro-1H-indol-1-yl)-1,3-dimethyl-1H-pyrazol-4-yl]but-2-enoate

By a method similar to that in Reference Example 12, the title compound was obtained from [5-(5-chloro-1H-indol-1-yl)-1,3-dimethyl-1H-pyrazol-4-yl]acetaldehyde obtained in Reference Example 62 and ethyl (diethoxyphosphoryl)acetate.

¹H-NMR (300 MHz, CDCl₃) δ:1.18-1.29 (m, 3H), 2.25 (s, 3H), 2.96-3.17 (m, 2H), 3.49 (s, 3H), 4.07-4.17 (m, 2H), 5.49-5.60 (m, 1H), 6.65 (dd, J=3.3, 0.8 Hz, 1H), 6.71-6.82 (m, 1H), 6.92 (d, J=8.7 Hz, 1H), 7.05 (d, J=3.4 Hz, 1H), 7.18 (dd, J=8.7, 1.9 Hz, 1H), 7.64 (d, J=1.7 Hz, 1H).

### Reference Example 70

### (2E)-4-[5-(5-chloro-1H-indol-1-yl)-1,3-dimethyl-1H-pyrazol-4-yl]but-2-enoic acid

By a method similar to that in Reference Example 13, the title compound was obtained from ethyl (2E)-4-[5-(5-chloro-1H-indol-1-yl)-1,3-dimethyl-1H-pyrazol-4-yl]but-2-enoate obtained in Reference Example 69.

¹H-NMR (300 MHz, DMSO-d₆) δ: 2.30 (s, 3H), 3.37-3.52 (m, 5H), 5.32-5.49 (m, 1H), 5.88-5.98 (m, 1H), 6.72-6.83 (m, 1H), 6.96-7.07 (m, 1H), 7.14-7.23 (m, 1H), 7.51-7.65 (m, 1H), 7.70-7.80 (m, 1H), 12.11 (br s, 1H).

### Reference Example 71

### 4-[5-(5-chloro-1H-indol-1-yl)-1,3-dimethyl-1H-pyrazol-4-yl]butanoic acid

By a method similar to that in Reference Example 65, the title compound was obtained from (2E)-4-[5-(5-chloro-1H-indol-1-yl)-1,3-dimethyl-1H-pyrazol-4-yl]but-2-enoic acid obtained in Reference Example 70.

¹H-NMR (300 MHz, DMSO-d₆) δ: 1.34-1.47 (m, 2H), 1.95-2.32 (m, 7H), 3.37 (s, 3H), 6.75 (d, J=2.7 Hz, 1H), 7.01 (d, J=8.7 Hz, 1H), 7.19 (dd, J=8.7, 1.9 Hz, 1H), 7.55 (d, J=3.4 Hz, 1H), 7.74 (d, J=1.9 Hz, 1H).

### Reference Example 72

### 2-{[5-(5-chloro-1H-indol-1-yl)-1,3-dimethyl-1H-pyrazol-4-yl]methyl}-1H-isoindole-1,3(2H)-dione

To a solution of [5-(5-chloro-1H-indol-1-yl)-1,3-dimethyl-1H-pyrazol-4-yl]methanol obtained in Reference Example 61 (3.52 g) in tetrahydrofuran (25 mL) were added methanesulfonyl chloride (2.05 g) and triethylamine (1.94 g) with stirring at 0° C., and the mixture was stirred at room temperature for 16 hr. Water was added to this reaction mixture, and the mixture was extracted with ethyl acetate. The organic layer was washed with saturated brine, dried over anhydrous magnesium sulfate, and filtrated. The filtrate was concentrated, and the residue was dissolved in N,N-dimethylformamide (15 mL). Potassium phthalimide (2.07 g) was added, and the mixture was stirred at 50° C. for 16 hr. Water was added to this reaction mixture, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (3.23 g, yield 62%) as colorless crystals.

¹H-NMR (300 MHz, CDCl₃) δ: 2.37 (s, 3H), 3.41 (s, 3H), 4.38 (d, J=15.3 Hz, 1H), 4.73 (d, J=15.3 Hz, 1H), 6.59 (dd, J=3.4, 0.8 Hz, 1H), 6.73 (d, J=8.7 Hz, 1H), 6.89 (dd, J=8.7, 1.9 Hz, 1H), 7.23 (d, J=3.4 Hz, 1H), 7.46 (d, J=1.7 Hz, 1H), 7.55-7.65 (m, 4H).

### Reference Example 73

### 2-{2-[5-(5-chloro-1H-indol-1-yl)-1,3-dimethyl-1H-pyrazol-4-yl]ethyl}-1H-isoindole-1,3(2H)-dione

By a method similar to that in Reference Example 72, the title compound was obtained from 2-[5-(5-chloro-1H-indol-1-yl)-1,3-dimethyl-1H-pyrazol-4-yl]ethanol obtained in Reference Example 63.

¹H-NMR (300 MHz, CDCl₃) δ: 2.23 (s, 3H), 2.50-2.84 (m, 2H), 3.42 (s, 3H), 3.50-3.75 (m, 2H), 6.65 (dd, J=3.3, 0.8 Hz, 1H), 6.80-6.96 (m, 2H), 7.19 (d, J=3.2 Hz, 1H), 7.57 (d, J=1.7 Hz, 1H), 7.68 (s, 4H).

### Reference Example 74

### 2-{3-[5-(5-chloro-1H-indol-1-yl)-1,3-dimethyl-1H-pyrazol-4-yl]propyl}-1H-isoindole-1,3(2H)-dione

By a method similar to that in Reference Example 72, the title compound was obtained from 3-[5-(5-chloro-1H-indol-1-yl)-1,3-dimethyl-1H-pyrazol-4-yl]propan-1-ol obtained in Reference Example 66.

¹H-NMR (300 MHz, CDCl₃) δ: 1.55-1.73 (m, 2H), 2.15-2.54 (m, 5H), 3.42-3.56 (m, 5H), 6.58 (d, J=3.4 Hz, 1H), 6.88-6.97 (m, 1H), 7.06-7.14 (m, 2H), 7.52 (d, J=1.7 Hz, 1H), 7.65-7.97 (m, 4H).

### Reference Example 75

### 1-[5-(5-chloro-1H-indol-1-yl)-1,3-dimethyl-1H-pyrazol-4-yl]methanamine

To a solution of 2-{[5-(5-chloro-1H-indol-1-yl)-1,3-dimethyl-1H-pyrazol-4-yl]methyl}-1H-isoindole-1,3(2H)-dione obtained in Reference Example 72 (543 mg) in tetrahydrofuran (13 mL) was added a 35% aqueous hydrazine solution (1.23 g) with stirring, and the mixture was heated under reflux for 16 hr. After the reaction mixture was allowed to cool to room temperature, and concentrated, water was added to the residue, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (305 mg, yield 83%) as colorless crystals.

¹H-NMR (300 MHz, CDCl₃) δ: 1.25 (br s, 2H), 2.35 (s, 3H), 3.40-3.55 (m, 5H), 6.68 (d, J=2.3 Hz, 1H), 6.97 (d, J=8.7 Hz, 1H), 7.14 (d, J=3.4 Hz, 1H), 7.19 (dd, J=8.7, 1.9 Hz, 1H), 7.66 (d, J=1.9 Hz, 1H).

### Reference Example 76

### 2-[5-(5-chloro-1H-indol-1-yl)-1,3-dimethyl-1H-pyrazol-4-yl]ethanamine

By a method similar to that in Reference Example 75, the title compound was obtained from 2-{2-[5-(5-chloro-1H-indol-1-yl)-1,3-dimethyl-1H-pyrazol-4-yl]ethyl}-1H-isoindole-1,3(2H)-dione obtained in Reference Example 73.

¹H-NMR (300 MHz, CDCl₃) δ: 1.12-1.32 (m, 2H), 2.24-2.44 (m, 5H), 2.60 (br s, 2H), 3.46 (s, 3H), 6.67 (dd, J=3.3, 0.8 Hz, 1H), 6.94 (d, J=8.7 Hz, 1H), 7.12 (d, J=3.2 Hz, 1H), 7.15-7.20 (m, 1H), 7.65 (d, J=1.7 Hz, 1H).

### Reference Example 77

### 3-[5-(5-chloro-1H-indol-1-yl)-1,3-dimethyl-1H-pyrazol-4-yl]propan-1-amine

By a method similar to that in Reference Example 75, the title compound was obtained from 2-{3-[5-(5-chloro-1H-indol-1-yl)-1,3-dimethyl-1H-pyrazol-4-yl]propyl}-1H-isoindole-1,3(2H)-dione obtained in Reference Example 74.

¹H-NMR (300 MHz, CDCl₃) δ: 1.07-1.23 (m, 2H), 1.34-1.46 (m, 2H), 2.15-2.36 (m, 5H), 2.50 (br s, 2H), 3.45 (s, 3H), 6.67 (dd, J=3.3, 0.8 Hz, 1H), 6.94 (d, J=8.7 Hz, 1H), 7.10 (d, J=3.2 Hz, 1H), 7.15-7.21 (m, 1H), 7.65 (d, J=1.9 Hz, 1H).

### Reference Example 78

### 3-[5-(5-chloro-1H-indol-1-yl)-1,3-dimethyl-1H-pyrazol-4-yl]propanal

To a mixture of Celite® (4.00 g) in dichloromethane (35 mL) was added pyridinium dichromate (4.00 g) with stirring, and the mixture was stirred at room temperature for 10 min. 3-[5-(5-Chloro-1H-indol-1-yl)-1,3-dimethyl-1H-pyrazol-4-yl]propan-1-ol obtained in Reference Example 66 (2.70 g) was added to this reaction mixture, and the mixture was stirred at room temperature for 18 hr. The reaction mixture was filtrated through Celite®, and the filtrate was concentrated. The residue was subjected to silica gel column chromatography (hexane-ethyl acetate 50:50, v/v) to give the title compound (1.49 g, yield 56%) as a pale-yellow oil.

¹H-NMR (300 MHz, CDCl₃) δ: 2.31 (s, 3H), 2.35 (dd, J=5.7, 1.9 Hz, 2H), 2.52-2.61 (m, 2H), 3.44 (s, 3H), 6.69 (d, J=3.4 Hz, 1H), 6.92 (d, J=8.7 Hz, 1H), 7.10 (d, J=3.0 Hz, 1H), 7.17-7.22 (m, 1H), 7.66 (d, J=1.9 Hz, 1H), 9.53 (s, 1H).

### Reference Example 79

### 1-(4-formyl-1,3-dimethyl-1H-pyrazol-5-yl)-1H-indole-5-carbonitrile

By a method similar to that in Reference Example 1, the title compound was obtained from 1H-indole-5-carbonitrile and 5-chloro-1,3-dimethyl-1H-pyrazole-4-carbaldehyde.

¹H-NMR (300 MHz, CDCl₃) δ:2.56 (s, 3H), 3.58 (s, 3H), 6.90 (d, J=3.3 Hz, 1H), 7.15 (d, J=8.7 Hz, 1H), 7.31 (d, J=3.3 Hz, 1H), 7.53 (dd, J=8.7, 1.6 Hz, 1H), 8.07 (d, J=1.6 Hz, 1H), 9.54 (s, 1H).

### Reference Example 80

### ethyl (2E)-3-[5-(5-cyano-1H-indol-1-yl)-1,3-dimethyl-1H-pyrazol-4-yl]acrylate

By a method similar to that in Reference Example 12, the title compound was obtained from 1-(4-formyl-1,3-dimethyl-1H-pyrazol-5-yl)-1H-indole-5-carbonitrile obtained in Reference Example 79 and ethyl (diethoxyphosphoryl)acetate.

¹H-NMR (300 MHz, CDCl₃) δ:1.19-1.25 (m, 3H), 2.47 (s, 3H), 3.51 (s, 3H), 4.08-4.17 (m, 2H), 5.60 (d, J=16.2 Hz, 1H), 6.90 (dd, J=3.4, 0.8 Hz, 1H), 7.08 (d, J=8.5 Hz, 1H), 7.21-7.28 (m, 2H), 7.50 (dd, J=8.5, 1.5 Hz, 1H), 8.09 (d, J=0.9 Hz, 1H).

### Reference Example 81

### (2E)-3-[5-(5-cyano-1H-indol-1-yl)-1,3-dimethyl-1H-pyrazol-4-yl]acrylic acid

By a method similar to that in Reference Example 13, the title compound was obtained from ethyl (2E)-3-[5-(5-cyano-1H-indol-1-yl)-1,3-dimethyl-1H-pyrazol-4-yl]acrylate obtained in Reference Example 80.

¹H-NMR (300 MHz, DMSO-d₆) δ: 2.37 (s, 3H), 3.48 (s, 3H), 5.38 (d, J=16.2 Hz, 1H), 6.92-7.02 (m, 2H), 7.21 (d, J=8.5 Hz, 1H), 7.58 (dd, J=8.7, 1.3 Hz, 1H), 7.81 (d, J=3.2 Hz, 1H), 8.29 (s, 1H).

### Reference Example 82

### 5-(6-fluoro-1H-indol-1-yl)-1,3-dimethyl-1H-pyrazole-4-carbaldehyde

By a method similar to that in Reference Example 1, the title compound was obtained from 6-fluoro-1H-indole and 5-chloro-1,3-dimethyl-1H-pyrazole-4-carbaldehyde.

¹H-NMR (300 MHz, CDCl₃) δ:2.56 (s, 3H), 3.59 (s, 3H), 6.76-6.82 (m, 2H), 6.97-7.05 (m, 1H), 7.17 (d, J=3.4 Hz, 1H), 7.63 (dd, J=8.7, 5.3 Hz, 1H), 9.54 (s, 1H).

### Reference Example 83

### (2E)-3-[5-(6-fluoro-1H-indol-1-yl)-1,3-dimethyl-1H-pyrazol-4-yl]acrylic acid

By a method similar to that in Reference Example 2, the title compound was obtained from 5-(6-fluoro-1H-indol-1-yl)-1,3-dimethyl-1H-pyrazole-4-carbaldehyde obtained in Reference Example 82 and malonic acid.

¹H-NMR (300 MHz, DMSO-d₆) δ: 2.38 (s, 3H), 3.49 (s, 3H), 5.33-5.42 (m, 1H), 6.84-6.91 (m, 2H), 7.01-7.11 (m, 2H), 7.57 (d, J=3.4 Hz, 1H), 7.73 (dd, J=8.5, 5.5 Hz, 1H), 12.17 (br s, 1H).

### Reference Example 84

### 5-(methoxymethyl)-2-methyl-2,4-dihydro-3H-pyrazol-3-one

To a solution of methyl 4-methoxyacetacetate (3.48 g) in toluene (110 mL) was added dropwise a solution of methylhydrazine (1.10 g) in toluene (35 mL) over 20 min at 0° C., and the mixture was stirred at 100° C. for 1.5 hr. The reaction mixture was allowed to cool to room temperature, and concentrated under reduced pressure. The residue was crystallized from diisopropyl ether-hexane to give the title compound (3.20 g, yield 95%) as brown crystals.

¹H-NMR (300 MHz, CDCl₃) δ:3.29 (s, 2H), 3.31 (s, 3H), 3.39 (s, 3H), 4.17 (s, 2H).

### Reference Example 85

### 5-chloro-3-(methoxymethyl)-1-methyl-1H-pyrazole-4-carbaldehyde

Phosphoryl chloride (201 g) was added dropwise over 30 min to N,N-dimethylformamide (31.9 g) cooled at 0° C. 5-(Methoxymethyl)-2-methyl-2,4-dihydro-3H-pyrazol-3-one obtained in Reference Example 84 (31.0 g) was added to this reaction mixture, and the mixture was stirred with heating at 80° C. for 3 hr. The reaction mixture was allowed to cool to room temperature, and poured into ice water, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 90:10-50:50, v/v) to give the title compound (23.5 g, yield 57%) as a colorless oil.

¹H-NMR (300 MHz, CDCl₃) δ:3.48 (s, 3H), 3.88 (s, 3H), 4.69 (s, 2H), 9.90 (s, 1H).

### Reference Example 86

### 5-(5-fluoro-1H-indol-1-yl)-3-(methoxymethyl)-1-methyl-1H-pyrazole-4-carbaldehyde

By a method similar to that in Reference Example 1, the title compound was obtained from 5-fluoro-1H-indole and 5-chloro-3-(methoxymethyl)-1-methyl-1H-pyrazole-4-carbaldehyde obtained in Reference Example 85.

¹H-NMR (300 MHz, CDCl₃) δ:3.55 (s, 3H), 3.64 (s, 3H), 4.78 (s, 2H), 6.79 (d, J=3.4 Hz, 1H), 6.97-7.05 (m, 2H), 7.23 (d, J=3.4 Hz, 1H), 7.33-7.40 (m, 1H), 9.59 (s, 1H).

### Reference Example 87

### ethyl (2E)-3-[5-(5-fluoro-1H-indol-1-yl)-3-(methoxymethyl)-1-methyl-1H-pyrazol-4-yl]acrylate

By a method similar to that in Reference Example 12, the title compound was obtained from 5-(5-fluoro-1H-indol-1-yl)-3-(methoxymethyl)-1-methyl-1H-pyrazole-4-carbaldehyde obtained in Reference Example 86 and ethyl (diethoxyphosphoryl)acetate.

¹H-NMR (300 MHz, CDCl₃) δ:1.18-1.26 (m, 3H), 3.50 (s, 3H), 3.55 (s, 3H), 4.13 (q, J=7.2 Hz, 2H), 4.54-4.64 (m, 2H), 5.76 (d, J=16.2 Hz, 1H), 6.77 (d, J=3.2 Hz, 1H), 6.88-7.04 (m, 2H), 7.13 (d, J=3.2 Hz, 1H), 7.31 (d, J=16.2 Hz, 1H), 7.36 (dd, J=9.1, 2.4 Hz, 1H).

### Reference Example 88

### (2E)-3-[5-(5-fluoro-1H-indol-1-yl)-3-(methoxymethyl)-1-methyl-1H-pyrazol-4-yl]acrylic acid

By a method similar to that in Reference Example 13, the title compound was obtained from ethyl (2E)-3-[5-(5-fluoro-1H-indol-1-yl)-3-(methoxymethyl)-1-methyl-1H-pyrazol-4-yl]acrylate obtained in Reference Example 87.

¹H-NMR (300 MHz, DMSO-d₆) δ: 3.34 (s, 3H), 3.55 (s, 3H), 4.47-4.59 (m, 2H), 5.47 (d, J=16.2 Hz, 1H), 6.87 (d, J=3.4 Hz, 1H), 6.98-7.15 (m, 3H), 7.52 (dd, J=9.5, 2.2 Hz, 1H), 7.69 (d, J=3.2 Hz, 1H), 12.22 (br s, 1H).

### Reference Example 89

### (2E)-3-[5-(5-chloro-1H-indol-1-yl)-1,3-dimethyl-1H-pyrazol-4-yl]acrylonitrile

To a solution of diethyl (cyanomethyl)phosphonate (1.07 g) in tetrahydrofuran (22 mL), which was cooled at 0° C. in an ice bath, was added 60% sodium hydride (in oil, 263 mg) with stirring, and the mixture was stirred at 0° C. for 30 min. A solution of 5-(5-chloro-1H-indol-1-yl)-1,3-dimethyl-1H-pyrazole-4-carbaldehyde obtained in Reference Example 37 (1.50 g) in tetrahydrofuran (8 mL) was added to this reaction mixture at 0° C., and the reaction mixture was stirred at room temperature for 4 hr. Water was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30, v/v) to give the title compound (1.49 g, yield 92%) as a colorless amorphous solid.

¹H-NMR (300 MHz, CDCl₃) δ:2.41 (s, 3H), 3.54 (s, 3H), 4.83 (d, J=17.0 Hz, 1H), 6.77 (d, J=3.2 Hz, 1H), 6.88-6.98 (m, 2H), 7.10 (d, J=3.4 Hz, 1H), 7.23 (dd, J=8.8, 2.0 Hz, 1H), 7.70 (d, J=1.9 Hz, 1H).

### Reference Example 90

### (1Z,2E)-3-[5-(5-chloro-1H-indol-1-yl)-1,3-dimethyl-1H-pyrazol-4-yl]-N′-hydroxyprop-2-enimidamide

To a solution of (2E)-3-[5-(5-chloro-1H-indol-1-yl)-1,3-dimethyl-1H-pyrazol-4-yl]acrylonitrile obtained in Reference Example 89 (321 mg) in dimethylsulfoxide (11 mL) were added hydroxylammonium chloride (377 mg) and triethylamine (549 mg) with stirring, and the mixture was stirred at 75° C. for 3 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 35:65, v/v) to give the title compound (159 mg, yield 44%) as colorless crystals.

¹H-NMR (300 MHz, CDCl₃) δ:2.44 (s, 3H), 3.49 (s, 3H), 4.42 (br s, 2H), 5.73 (d, J=17.0 Hz, 1H), 6.34 (d, J=17.0 Hz, 1H), 6.73 (d, J=2.7 Hz, 1H), 6.94 (d, J=8.7 Hz, 1H), 7.12 (d, J=2.7 Hz, 1H), 7.20 (d, J=9.1 Hz, 1H), 7.67 (s, 1H).

### Reference Example 91

### 5-chloro-1-{4-[(2,2-diethoxyethoxy)methyl]-1,3-dimethyl-1H-pyrazol-5-yl}-1H-indole

To a solution of [5-(5-chloro-1H-indol-1-yl)-1,3-dimethyl-1H-pyrazol-4-yl]methanol obtained in Reference Example 61 (1.12 g) in N,N-dimethylformamide (8 mL) was added 60% sodium hydride (in oil, 195 mg) with stirring, and the mixture was stirred at 0° C. for 30 min. 2-Bromo-1,1-diethoxyethane (1.20 g) was added to this reaction mixture, and the mixture was stirred at 80° C. for 72 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 75:25, v/v) to give the title compound (1.05 g, yield 66%) as a colorless oil.

¹H-NMR (300 MHz, CDCl₃) δ:1.14 (t, J=7.0 Hz, 6H), 2.35 (s, 3H), 3.31 (d, J=4.9 Hz, 2H), 3.36-3.65 (m, 7H), 4.08-4.26 (m, 2H), 4.46 (t, J=5.3 Hz, 1H), 6.66 (d, J=3.0 Hz, 1H), 6.99 (d, J=8.7 Hz, 1H), 7.14-7.21 (m, 2H), 7.65 (d, J=1.9 Hz, 1H).

### Reference Example 92

### {[5-(5-chloro-1H-indol-1-yl)-1,3-dimethyl-1H-pyrazol-4-yl]methoxy}acetaldehyde

To a solution of 5-chloro-1-{4-[(2,2-diethoxyethoxy)methyl]-1,3-dimethyl-1H-pyrazol-5-yl}-1H-indole obtained in Reference Example 91 (1.03 g) in tetrahydrofuran (5.3 mL) was added 1N hydrochloric acid (5.3 mL), and the mixture was stirred at 50° C. for 2.5 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v) to give the title compound (643 mg, yield 77%) as a pale-yellow amorphous solid.

¹H-NMR (300 MHz, CDCl₃) δ:2.38 (s, 3H), 3.46-3.53 (m, 5H), 3.89 (dd, J=1.8, 0.8 Hz, 2H), 6.69 (dd, J=3.3, 0.8 Hz, 1H), 6.98 (d, J=8.7 Hz, 1H), 7.16-7.22 (m, 2H), 7.66 (d, J=1.5 Hz, 1H), 9.53 (t, J=0.9 Hz, 1H).

### Reference Example 93

### {[5-(5-chloro-1H-indol-1-yl)-1,3-dimethyl-1H-pyrazol-4-yl]methoxy}acetic acid

By a method similar to that in Reference Example 67, the title compound was obtained from {[5-(5-chloro-1H-indol-1-yl)-1,3-dimethyl-1H-pyrazol-4-yl]methoxy}acetaldehyde obtained in Reference Example 92.

¹H-NMR (300 MHz, DMSO-d₆) δ: 2.26 (s, 3H), 3.39-3.45 (m, 5H), 3.81 (s, 2H), 6.76 (dd, J=3.2, 0.8 Hz, 1H), 7.07-7.13 (m, 1H), 7.16-7.22 (m, 1H), 7.56-7.61 (m, 1H), 7.74 (d, J=1.7 Hz, 1H).

### Reference Example 94

### (3E)-4-[5-(5-chloro-1H-indol-1-yl)-1,3-dimethyl-1H-pyrazol-4-yl]-2-oxobut-3-enoic acid

5-(5-Chloro-1H-indol-1-yl)-1,3-dimethyl-1H-pyrazole-4-carbaldehyde obtained in Reference Example 37 (1.03 g) was dissolved in a mixed solvent of methanol (18 mL) and water (18 mL), pyruvic acid (1.32 g) and sodium carbonate (1.59 g) were added, and the mixture was heated under reflux for 8 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was crystallized from hexane-ethanol to give the title compound (750 mg, yield 58%) as pale-yellow crystals.

¹H-NMR (300 MHz, DMSO-d₆) δ: 2.43 (s, 3H), 3.52 (s, 3H), 6.22 (d, J=16.7 Hz, 1H), 6.88 (d, J=3.0 Hz, 1H), 7.08 (d, J=8.7 Hz, 1H), 7.15-7.26 (m, 2H), 7.70 (d, J=3.4 Hz, 1H), 7.81 (d, J=1.9 Hz, 1H), 13.92 (br s, 1H).

### Reference Example 95

### ethyl (3E)-4-[5-(5-chloro-1H-indol-1-yl)-1,3-dimethyl-1H-pyrazol-4-yl]-2-oxobut-3-enoate

To a solution of (3E)-4-[5-(5-chloro-1H-indol-1-yl)-1,3-dimethyl-1H-pyrazol-4-yl]-2-oxobut-3-enoic acid obtained in Reference Example 94 (545 mg) in ethanol (10 mL) was added hydrochloric acid (0.5 mL), and the mixture was heated under reflux for 18 hr. The reaction mixture was allowed to cool to room temperature, and concentrated under reduced pressure. The residue was subjected to silica gel column chromatography (hexane-ethyl acetate 50:50, v/v) to give the title compound (403 mg, yield 68%) as a pale-yellow oil.

¹H-NMR (300 MHz, CDCl₃) δ:1.22-1.29 (m, 3H), 2.50 (s, 3H), 3.56 (s, 3H), 4.18-4.28 (m, 2H), 6.43 (d, J=16.4 Hz, 1H), 6.79 (dd, J=3.3, 0.8 Hz, 1H), 6.92 (d, J=8.9 Hz, 1H), 7.13 (d, J=3.4 Hz, 1H), 7.19-7.24 (m, 1H), 7.41-7.50 (m, 1H), 7.70 (d, J=1.7 Hz, 1H).

### Reference Example 96

### ethyl 4-[5-(5-chloro-1H-indol-1-yl)-1,3-dimethyl-1H-pyrazol-4-yl]-2-hydroxybutanoate

Ethyl (3E)-4-[5-(5-chloro-1H-indol-1-yl)-1,3-dimethyl-1H-pyrazol-4-yl]-2-oxobut-3-enoate obtained in Reference Example 95 (403 mg) was dissolved in a mixed solvent of tetrahydrofuran (15 mL) and ethanol (15 mL), 10% palladium carbon (42 mg) was added, and the mixture was stirred under 1 atom of hydrogen atmosphere at room temperature for 16 hr. The catalyst was removed by filtration, and the filtrate was concentrated. The residue was dissolved in a mixed solvent of tetrahydrofuran (15 mL) and ethanol (15 mL), sodium borohydride (41 mg) was added, and the mixture was stirred at 0° C. for 3 hr. Water was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v) to give the title compound (251 mg, yield 62%) as a pale-yellow oil.

¹H-NMR (300 MHz, CDCl₃) δ:1.08-1.18 (m, 3H), 1.48-1.78 (m, 2H), 2.19-2.51 (m, 5H), 2.65 (dd, J=10.6, 5.2 Hz, 1H), 3.41-3.47 (m, 3H), 3.95-4.16 (m, 2H), 6.65-6.69 (m, 1H), 6.94 (dd, J=8.8, 2.5 Hz, 1H), 7.12 (dd, J=5.0, 3.3 Hz, 1H), 7.15-7.20 (m, 1H), 7.65 (d, J=1.7 Hz, 1H).

### Reference Example 97

### ethyl 2-chloro-4-[5-(5-chloro-1H-indol-1-yl)-1,3-dimethyl-1H-pyrazol-4-yl]butanoate

To a solution of ethyl 4-[5-(5-chloro-1H-indol-1-yl)-1,3-dimethyl-1H-pyrazol-4-yl]-2-hydroxybutanoate obtained in Reference Example 96 (241 mg) in tetrahydrofuran (7 mL) was added thionyl chloride (229 mg), and the mixture was heated under reflux for 22 hr. The reaction mixture was allowed to cool to room temperature, and concentrated. The residue was purified by preparative HPLC (tool: Gilson, Inc., high through-put purification system, column: YMC Combiprep ODS-A, S-5 μm, 50×20 mm, solvent: SOLUTION A; 0.1% trifluoroacetic acid-containing water, SOLUTION B; 0.1% trifluoroacetic acid-containing acetonitrile, gradient cycle: 0.00 min (SOLUTION A/SOLUTION B=90/10), 1.00 min (SOLUTION A/SOLUTION B=90/10), 4.20 min (SOLUTION A/SOLUTION B=10/90), 5.40 min (SOLUTION A/SOLUTION B=10/90), 5.50 min (SOLUTION A/SOLUTION B=90/10), 5.60 min (SOLUTION A/SOLUTION B=90/10), flow rate: 25 mL/min, detection method: UV 220 nm) to give the title compound (93.7 mg, yield 37%) as a colorless oil.

¹H-NMR (300 MHz, CDCl₃) δ:1.13-1.23 (m, 3H), 1.81-1.94 (m, 2H), 2.30 (s, 3H), 2.33-2.52 (m, 2H), 3.46 (s, 3H), 4.00-4.17 (m, 3H), 6.68 (d, J=3.4 Hz, 1H), 6.92 (dd, J=8.7, 1.9 Hz, 1H), 7.11 (d, J=3.0 Hz, 1H), 7.19 (d, J=8.7 Hz, 1H), 7.66 (d, J=1.9 Hz, 1H).

### Reference Example 98

### sodium cyclopropylmethanesulfonate

(Bromomethyl)cyclopropane (3.00 g) was added to an saturated aqueous sodium sulfite solution (27 mL), and the mixture was heated under reflux for 24 hr. The reaction mixture was allowed to cool to room temperature, and concentrated under reduced pressure. Ethanol was added to the residue, and the mixture was stirred at 50° C. for 30 min. The mixture was filtrated, and the filtrate was concentrated. Toluene was added to the residue, and the mixture was concentrated again under reduced pressure. The residue was dried to give the title compound (2.54 g, yield 72%) as colorless crystals.

¹H-NMR (300 MHz, DMSO-d₆) δ: 0.12-0.19 (m, 2H), 0.36-0.45 (m, 2H), 0.86-0.99 (m, 1H), 2.32 (d, J=6.8 Hz, 2H).

### Reference Example 99

### sodium 4-methylpentane-1-sulfonate

By a method similar to that in Reference Example 98, the title compound was obtained from 1-bromo-4-methylpentane.

¹H-NMR (300 MHz, DMSO-d₆) δ: 0.85 (d, J=6.6 Hz, 6H), 1.11-1.26 (m, 2H), 1.43-1.62 (m, 3H), 2.31-2.41 (m, 2H).

### Reference Example 100

### 1-cyclopropylmethanesulfonamide

Sodium cyclopropylmethanesulfonate obtained in Reference Example 98 (961 mg) was dissolved in a mixed solvent of N,N-dimethylformamide (0.5 mL) and tetrahydrofuran (12 mL), thionyl chloride (1.45 g) was added, and the mixture was heated under reflux for 3 hr. The reaction mixture was allowed to cool to room temperature, and filtrated, and the filtrate was concentrated. The residue was dissolved in tetrahydrofuran (12 mL), and the solution was added to 35% aqueous ammonia (6 mL) at 0° C. The reaction mixture was extracted with ethyl acetate. The organic layer was washed with saturated brine, dried over anhydrous magnesium sulfate, and filtrated. The filtrate was concentrated to give the title compound (579 mg, yield 70%) as a pale-yellow oil.

¹H-NMR (300 MHz, DMSO-d₆) δ: 0.28-0.37 (m, 2H), 0.51-0.64 (m, 2H), 0.92-1.08 (m, 1H), 3.14-3.29 (m, 2H).

### Reference Example 101

### 4-methylpentane-1-sulfonamide

By a method similar to that in Reference Example 100, the title compound was obtained from sodium 4-methylpentane-1-sulfonate obtained in Reference Example 99.

¹H-NMR (300 MHz, DMSO-d₆) δ: 0.87 (d, J=6.8 Hz, 6H), 1.13-1.31 (m, 2H), 1.46-1.75 (m, 3H), 2.87-3.00 (m, 2H).

### Reference Example 102

### benzyl (morpholin-4-ylsulfonyl)carbamate

To a solution of benzyl alcohol (3.00 g) in acetonitrile (200 mL) was added chlorosulfonyl isocyanate (4.70 g) with stirring at 0° C., and the mixture was stirred at 0° C. for 30 min. Pyridine (6.58 g) was added to this reaction mixture, and the mixture was stirred at 0° C. for 1 hr. Morpholine (9.67 g) was added to the reaction mixture, and the mixture was stirred at room temperature for 5 hr. Water was added to this reaction mixture, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (9.23 g, yield 99%) as colorless crystals.

¹H-NMR (300 MHz, DMSO-d₆) δ:3.15-3.21 (m, 4H), 3.57-3.63 (m, 4H), 5.15 (s, 2H), 7.31-7.45 (m, 5H), 11.50 (br s, 1H).

### Reference Example 103

### benzyl {[methyl(pentyl)amino]sulfonyl}carbamate

By a method similar to that in Reference Example 102, the title compound was obtained from benzyl alcohol, chlorosulfonyl isocyanate and N-methylpentan-1-amine.

¹H-NMR (300 MHz, CDCl₃) δ:0.89 (t, J=6.8 Hz, 3H), 1.16-1.38 (m, 4H), 1.48-1.61 (m, 2H), 2.90 (s, 3H), 3.17-3.26 (m, 2H), 5.17 (s, 2H), 7.32-7.39 (m, 5H).

### Reference Example 104

### benzyl [(butylamino)sulfonyl]carbamate

By a method similar to that in Reference Example 102, the title compound was obtained from benzyl alcohol, chlorosulfonyl isocyanate and butan-1-amine.

¹H-NMR (300 MHz, CDCl₃) δ:0.89 (t, J=7.4 Hz, 3H), 1.25-1.40 (m, 2H), 1.42-1.57 (m, 2H), 2.98-3.09 (m, 2H), 5.12-5.17 (m, 1H), 5.18 (s, 2H), 7.29-7.43 (m, 5H), 7.50 (br s, 1H).

### Reference Example 105

### benzyl {[(1-propylbutyl)amino]sulfonyl}carbamate

By a method similar to that in Reference Example 102, the title compound was obtained from benzyl alcohol, chlorosulfonyl isocyanate and heptan-4-amine.

¹H-NMR (300 MHz, CDCl₃) δ:0.86 (t, J=7.2 Hz, 6H), 1.19-1.51 (m, 8H), 3.27-3.42 (m, 1H), 4.73-4.91 (m, 1H), 5.19 (s, 2H), 7.33-7.46 (m, 5H).

### Reference Example 106

### benzyl {[(1-ethylpropyl)amino]sulfonyl}carbamate

By a method similar to that in Reference Example 102, the title compound was obtained from benzyl alcohol, chlorosulfonyl isocyanate and pentan-3-amine.

¹H-NMR (300 MHz, CDCl₃) δ:0.86 (t, J=7.4 Hz, 6H), 1.35-1.59 (m, 4H), 3.16-3.36 (m, 1H), 4.87 (d, J=7.3 Hz, 1H), 5.19 (s, 2H), 7.30 (br s, 1H), 7.32-7.43 (m, 5H).

### Reference Example 107

### benzyl [(cyclohexylamino)sulfonyl]carbamate

By a method similar to that in Reference Example 102, the title compound was obtained from benzyl alcohol, chlorosulfonyl isocyanate and cyclohexylamine.

¹H-NMR (300 MHz, CDCl₃) δ:0.95-1.37 (m, 6H), 1.48-1.74 (m, 2H), 1.77-1.94 (m, 2H), 2.97-3.33 (m, 1H), 4.97 (d, J=6.8 Hz, 1H), 5.20 (s, 2H), 7.29 (br s, 1H), 7.37 (s, 5H).

### Reference Example 108

### benzyl {[(cyclopropylmethyl)amino]sulfonyl}carbamate

To a solution of benzyl alcohol (12.08 g) in acetonitrile (250 mL) was added chlorosulfonyl isocyanate (9.75 mL) with stirring at 0° C., and the mixture was stirred at 0° C. for 30 min. Pyridine (17.9 mL) was added to this reaction mixture, and the mixture was stirred at 0° C. for 1 hr. Cyclopropylmethylamine (11.92 g) was added to the reaction mixture, and the mixture was stirred at room temperature for 5 hr. 1N Hydrochloric acid was added to this reaction mixture, and the mixture was extracted with ethyl acetate. The organic layer was washed with 1N hydrochloric acid and saturated brine, dried over anhydrous magnesium sulfate, and filtrated. The filtrate was concentrated, and the residue was crystallized from hexane-ethyl acetate to give the title compound (21.22 g, yield 67%) as colorless crystals.

¹H-NMR (300 MHz, CDCl₃) δ:0.10-0.18 (m, 2H), 0.45-0.54 (m, 2H), 0.82-1.04 (m, 1H), 2.90 (dd, J=7.2, 5.8 Hz, 2H), 5.19 (s, 2H), 5.22 (br s, 1H), 7.30 (br s, 1H), 7.33-7.44 (m, 5H).

### Reference Example 109

### morpholine-4-sulfonamide

Benzyl (morpholin-4-ylsulfonyl)carbamate obtained in Reference Example 102 (9.23 g) was dissolved in a mixed solvent of tetrahydrofuran (100 mL) and ethanol (100 mL), 10% palladium carbon (923 mg) was added, and the mixture was stirred under 1 atom of hydrogen atmosphere at room temperature for 4 hr. The catalyst was removed by filtration, and the filtrate was concentrated. The residue was crystallized from hexane-ethanol to give the title compound (4.93 g, yield 97%) as colorless crystals.

¹H-NMR (300 MHz, CDCl₃) δ:3.13-3.20 (m, 4H), 3.77-3.82 (m, 4H), 4.43 (br s, 2H).

### Reference Example 110

### N-methyl-N-pentylsulfamide

By a method similar to that in Reference Example 109, the title compound was obtained from benzyl {[methyl(pentyl)amino]sulfonyl}carbamate obtained in Reference Example 103.

¹H-NMR (300 MHz, CDCl₃) δ:0.87-0.94 (m, 3H), 1.23-1.40 (m, 4H), 1.53-1.65 (m, 2H), 2.80 (s, 3H), 3.06-3.14 (m, 2H), 4.59 (br s, 2H).

### Reference Example 111

### N-butylsulfamide

By a method similar to that in Reference Example 109, the title compound was obtained from benzyl [(butylamino)sulfonyl]carbamate obtained in Reference Example 104.

¹H-NMR (300 MHz, CDCl₃) δ:0.94 (t, J=7.3 Hz, 3H), 1.31-1.47 (m, 2H), 1.50-1.63 (m, 2H), 3.07-3.19 (m, 2H), 4.41 (br s, 1H), 4.63 (br s, 2H).

### Reference Example 112

### N-(1-propylbutyl)sulfamide

By a method similar to that in Reference Example 109, the title compound was obtained from benzyl {[(1-propylbutyl)amino]sulfonyl}carbamate obtained in Reference Example 105.

¹H-NMR (300 MHz, CDCl₃) δ:0.93 (t, J=7.2 Hz, 6H), 1.27-1.66 (m, 8H), 3.29-3.50 (m, 1H), 4.15 (br s, 1H), 4.50 (br s, 2H).

### Reference Example 113

### N-(1-ethylpropyl)sulfamide

By a method similar to that in Reference Example 109, the title compound was obtained from benzyl {[(1-ethylpropyl)amino]sulfonyl}carbamate obtained in Reference Example 106.

¹H-NMR (300 MHz, CDCl₃) δ:0.95 (t, J=7.4 Hz, 6H), 1.26-1.78 (m, 4H), 3.05-3.46 (m, 1H), 4.23 (d, J=8.1 Hz, 1H), 4.55 (br s, 2H).

### Reference Example 114

### N-cyclohexylsulfamide

By a method similar to that in Reference Example 109, the title compound was obtained from benzyl [(cyclohexylamino)sulfonyl]carbamate obtained in Reference Example 107.

¹H-NMR (300 MHz, CDCl₃) δ:1.06-1.49 (m, 6H), 1.49-1.83 (m, 2H), 1.93-2.14 (m, 2H), 3.09-3.42 (m, 1H), 4.51 (br s, 1H), 4.74 (br s, 2H).

### Reference Example 115

### N-(cyclopropylmethyl)sulfamide

Benzyl {[(cyclopropylmethyl)amino]sulfonyl}carbamate obtained in Reference Example 108 (20.30 g) was dissolved in a mixed solvent of tetrahydrofuran (150 mL) and ethanol (150 mL), 10% palladium carbon (30.39 g) was added, and the mixture was stirred under 1 atom of hydrogen atmosphere at room temperature for 6 hr. The catalyst was removed by filtration, and the filtrate was concentrated. The residue was crystallized from hexane-ethyl acetate to give the title compound (9.37 g, yield 87%) as colorless crystals.

¹H-NMR (300 MHz, CDCl₃) δ:0.17-0.31 (m, 2H), 0.49-0.64 (m, 2H), 0.94-1.18 (m, 1H), 3.00 (dd, J=7.2, 6.0 Hz, 2H), 4.40 (br s, 1H), 4.51 (br s, 2H).

### Reference Example 116

### ethyl N-({[(benzyloxy)carbonyl]amino}sulfonyl)glycinate

To a solution of benzyl alcohol (2.01 g) in acetonitrile (40 ml) was added chlorosulfonyl isocyanate (1.70 mL) with stirring at 0° C., and the mixture was stirred for 30 min. Pyridine (3.0 mL) was added to this reaction mixture, and the mixture was stirred at 0° C. for 1 hr. Glycine ethyl ester hydrochloride (3.90 g) and N,N-diisopropylethylamine (6.4 mL) were added, and the mixture was stirred at room temperature for 4 hr. 1N Hydrochloric acid was added to this reaction mixture, and the mixture was extracted with ethyl acetate. The organic layer was washed with 1N hydrochloric acid and saturated brine, dried over anhydrous magnesium sulfate, and filtrated. The filtrate was concentrated, and the residue was crystallized from hexane-ethyl acetate to give the title compound (5.70 g, yield 96%) as tetartohydrate colorless crystals.

¹H-NMR (300 MHz, CDCl₃) δ:1.28 (t, J=7.2 Hz, 3H), 3.96 (d, J=5.7 Hz, 2H), 4.21 (q, J=6.9 Hz, 2H), 5.21 (s, 2H), 5.61 (t, J=5.7 Hz, 1H), 7.31-7.49 (m, 5H).

### Reference Example 117

### ethyl N-(aminosulfonyl)glycinate

By a method similar to that in Reference Example 109, the title compound was obtained from ethyl N-({[(benzyloxy)carbonyl]amino}sulfonyl)glycinate obtained in Reference Example 116.

¹H-NMR (300 MHz, CDCl₃) δ:1.31 (t, J=7.2 Hz, 3H), 3.94 (d, J=5.7 Hz, 2H), 4.25 (q, J=7.1 Hz, 2H), 4.80 (br s, 2H), 5.09 (br s, 1H).

### Reference Example 118

### benzyl (1,4-dioxa-8-azaspiro[4.5]dec-8-ylsulfonyl)carbamate

By a method similar to that in Reference Example 102, the title compound was obtained from benzyl alcohol, chlorosulfonyl isocyanate and 1,4-dioxa-8-azaspiro[4.5]decane.

¹H-NMR (300 MHz, CDCl₃) δ:1.66-1.86 (m, 4H), 3.50 (t, J=5.8 Hz, 4H), 3.97 (s, 4H), 5.18 (s, 2H), 7.17 (br s, 1H), 7.30-7.44 (m, 5H).

### Reference Example 119

### benzyl {[(3-isopropoxypropyl)amino]sulfonyl}carbamate

By a method similar to that in Reference Example 102, the title compound was obtained from benzyl alcohol, chlorosulfonyl isocyanate and 3-isopropoxypropan-1-amine.

¹H-NMR (300 MHz, CDCl₃) δ:1.15 (d, J=6.0 Hz, 6H), 1.70-1.91 (m, 2H), 3.04-3.34 (m, 2H), 3.48 (t, J=5.6 Hz, 2H), 3.52-3.59 (m, 1H), 5.20 (s, 2H), 5.85 (t, J=5.6 Hz, 1H), 7.31 (br s, 1H), 7.34-7.42 (m, 5H).

### Reference Example 120

### 1,4-dioxa-8-azaspiro[4.5]decane-8-sulfonamide

By a method similar to that in Reference Example 109, the title compound was obtained from benzyl (1,4-dioxa-8-azaspiro[4.5]dec-8-ylsulfonyl)carbamate obtained in Reference Example 118.

¹H-NMR (300 MHz, CDCl₃) δ:1.83 (t, J=5.9 Hz, 4H), 3.32 (t, J=5.8 Hz, 4H), 3.98 (s, 4H), 4.38 (br s, 2H).

### Reference Example 121

### N-(3-isopropoxypropyl)sulfamide

By a method similar to that in Reference Example 109, the title compound was obtained from benzyl {[(3-isopropoxypropyl)amino]sulfonyl}carbamate obtained in Reference Example 119.

¹H-NMR (300 MHz, CDCl₃) δ:1.16 (d, J=6.0 Hz, 6H), 1.57-1.98 (m, 2H), 3.27 (t, J=6.1 Hz, 2H), 3.33-3.69 (m, 3H), 4.54 (br s, 2H), 5.15 (br s, 1H).

### Reference Example 122

### benzyl {[(cyclohexylmethyl)amino]sulfonyl}carbamate

By a method similar to that in Reference Example 102, the title compound was obtained from benzyl alcohol, chlorosulfonyl isocyanate and 1-cyclohexylmethanamine.

¹H-NMR (300 MHz, CDCl₃) δ:0.78-0.98 (m, 2H), 1.04-1.32 (m, 3H), 1.32-1.53 (m, 1H), 1.62-1.82 (m, 5H), 2.70-2.94 (m, 2H), 5.06 (t, J=6.1 Hz, 1H), 5.20 (s, 2H), 7.23 (br s, 1H), 7.31-7.43 (m, 5H).

### Reference Example 123

### N-(cyclohexylmethyl)sulfamide

By a method similar to that in Reference Example 109, the title compound was obtained from benzyl {[(cyclohexylmethyl)amino]sulfonyl}carbamate obtained in Reference Example 122.

¹H-NMR (300 MHz, CDCl₃) δ:0.83-1.05 (m, 2H), 1.11-1.35 (m, 3H), 1.42-1.55 (m, 1H), 1.62-1.84 (m, 5H), 2.90-3.03 (m, 2H), 4.30 (br s, 1H), 4.48 (br s, 2H).

### Reference Example 124

### benzyl {[(3-methylbutyl)amino]sulfonyl}carbamate

By a method similar to that in Reference Example 102, the title compound was obtained from benzyl alcohol, chlorosulfonyl isocyanate and 3-methylbutan-1-amine.

¹H-NMR (300 MHz, CDCl₃) δ:0.88 (d, J=6.6 Hz, 6H), 1.30-1.47 (m, 2H), 1.50-1.73 (m, 1H), 2.95-3.13 (m, 2H), 5.11 (br s, 1H), 5.19 (s, 2H), 7.38-7.56 (m, 6H).

### Reference Example 125

### N-(3-methylbutyl)sulfamide

By a method similar to that in Reference Example 109, the title compound was obtained from benzyl {[(3-methylbutyl)amino]sulfonyl}carbamate obtained in Reference Example 124.

¹H-NMR (300 MHz, CDCl₃) δ:0.92 (d, J=6.6 Hz, 6H), 1.36-1.54 (m, 2H), 1.59-1.75 (m, 1H), 3.05-3.18 (m, 2H), 4.32 (br s, 3H).

### Reference Example 126

### benzyl [(propylamino)sulfonyl]carbamate

By a method similar to that in Reference Example 102, the title compound was obtained from benzyl alcohol, chlorosulfonyl isocyanate and propan-1-amine.

¹H-NMR (300 MHz, DMSO-d₆) δ:0.81 (t, J=7.3 Hz, 3H), 1.12-1.56 (m, 2H), 2.75-2.86 (m, 2H), 5.14 (s, 2H), 7.14-7.41 (m, 5H), 7.76 (t, J=5.9 Hz, 1H), 11.20 (s, 1H).

### Reference Example 127

### N-propylsulfamide

By a method similar to that in Reference Example 109, the title compound was obtained from benzyl [(propylamino)sulfonyl]carbamate obtained in Reference Example 126.

¹H-NMR (300 MHz, CDCl₃) δ:0.97 (t, J=7.4 Hz, 3H), 1.55-1.67 (m, 2H), 3.09 (t, J=7.2 Hz, 2H), 4.47 (br s, 3H).

### Reference Example 128

### ethyl (2E)-3-[5-(1H-indol-1-yl)-1,3-dimethyl-1H-pyrazol-4-yl]acrylate

By a method similar to that in Reference Example 12, the title compound was obtained from 5-(1H-indol-1-yl)-1,3-dimethyl-1H-pyrazole-4-carbaldehyde obtained in Reference Example 1 and ethyl (diethoxyphosphoryl)acetate.

¹H-NMR (300 MHz, CDCl₃) δ:1.05-1.33 (m, 3H), 2.47 (s, 3H), 3.50 (s, 3H), 3.98-4.22 (m, 2H), 5.54-5.73 (m, 1H), 6.80 (d, J=2.7 Hz, 1H), 6.95-7.04 (m, 1H), 7.05-7.15 (m, 1H), 7.16-7.30 (m, 3H), 7.65-7.78 (m, 1H).

### Reference Example 129

### ethyl (2E)-3-[5-(3-chloro-1H-indol-1-yl)-1,3-dimethyl-1H-pyrazol-4-yl]acrylate

To a solution of ethyl (2E)-3-[5-(1H-indol-1-yl)-1,3-dimethyl-1H-pyrazol-4-yl]acrylate obtained in Reference Example 128 (5.00 g) in acetonitrile (75 mL) was added N-chlorosuccinimide (2.16 g) at room temperature, and the mixture was stirred at room temperature for 2 hr, and then at 50° C. for 2 hr. The reaction mixture was concentrated under reduced pressure, and the obtained residue was subjected to silica gel column chromatography (hexane-ethyl acetate 60:40-40:60, v/v), and crystallized from hexane-ethyl acetate to give the title compound (4.50 g, yield 81%) as colorless crystals.

¹H-NMR (300 MHz, CDCl₃) δ:1.23 (t, J=7.1 Hz, 3H), 2.47 (s, 3H), 3.50 (s, 3H), 4.13 (q, J=7.2 Hz, 2H), 5.71 (d, J=16.2 Hz, 1H), 6.96-7.01 (m, 1H), 7.10 (s, 1H), 7.27 (d, J=16.2 Hz, 1H), 7.29-7.33 (m, 2H), 7.65-7.77 (m, 1H).

### Reference Example 130

### (2E)-3-[5-(3-chloro-1H-indol-1-yl)-1,3-dimethyl-1H-pyrazol-4-yl]acrylic acid

By a method similar to that in Reference Example 13, the title compound was obtained from ethyl (2E)-3-[5-(3-chloro-1H-indol-1-yl)-1,3-dimethyl-1H-pyrazol-4-yl]acrylate obtained in Reference Example 129.

¹H-NMR (300 MHz, DMSO-d₆) δ:2.38 (s, 3H), 3.50 (s, 3H), 5.44 (d, J=16.2 Hz, 1H), 7.04 (d, J=16.2 Hz, 1H), 7.08-7.13 (m, 1H), 7.29-7.39 (m, 2H), 7.61-7.77 (m, 1H), 7.91 (s, 1H), 12.20 (br s, 1H).

### Reference Example 131

### 5-(3-chloro-1H-pyrrolo[2,3-b]pyridin-1-yl)-1,3-dimethyl-1H-pyrazole-4-carbaldehyde

By a method similar to that in Reference Example 1, the title compound was obtained from 3-chloro-1H-pyrrolo[2,3-b]pyridine and 5-chloro-1,3-dimethyl-1H-pyrazole-4-carbaldehyde.

¹H-NMR (300 MHz, CDCl₃) δ:2.55 (s, 3H), 3.69 (s, 3H), 7.28-7.34 (m, 2H), 8.06 (dd, J=8.0, 1.6 Hz, 1H), 8.41 (dd, J=4.9, 1.5 Hz, 1H), 9.62 (s, 1H).

### Reference Example 132

### ethyl (2E)-3-[5-(3-chloro-1H-pyrrolo[2,3-b]pyridin-1-yl)-1,3-dimethyl-1H-pyrazol-4-yl]acrylate

By a method similar to that in Reference Example 12, the title compound was obtained from 5-(3-chloro-1H-pyrrolo[2,3-b]pyridin-1-yl)-1,3-dimethyl-1H-pyrazole-4-carbaldehyde obtained in Reference Example 131 and ethyl (diethoxyphosphoryl)acetate.

¹H-NMR (300 MHz, DMSO-d₆) δ:1.14 (t, J=7.1 Hz, 3H), 2.39 (s, 3H), 3.52 (s, 3H), 4.05 (q, J=7.1 Hz, 2H), 5.60 (d, J=16.2 Hz, 1H), 7.09 (d, J=16.2 Hz, 1H), 7.41 (dd, J=7.9, 4.7 Hz, 1H), 8.09 (s, 1H), 8.18 (dd, J=8.0, 1.6 Hz, 1H), 8.39 (dd, J=4.7, 1.5 Hz, 1H).

### Reference Example 133

### (2E)-3-[5-(3-chloro-1H-pyrrolo[2,3-b]pyridin-1-yl)-1,3-dimethyl-1H-pyrazol-4-yl]acrylic acid

By a method similar to that in Reference Example 13, the title compound was obtained from ethyl (2E)-3-[5-(3-chloro-1H-pyrrolo[2,3-b]pyridin-1-yl)-1,3-dimethyl-1H-pyrazol-4-yl]acrylate obtained in Reference Example 132.

¹H-NMR (300 MHz, DMSO-d₆) δ:2.38 (s, 3H), 3.52 (s, 3H), 5.52 (d, J=16.2 Hz, 1H), 7.04 (d, J=16.2 Hz, 1H), 7.41 (dd, J=8.1, 4.7 Hz, 1H), 8.08 (s, 1H), 8.17 (dd, J=7.9, 1.5 Hz, 1H), 8.39 (dd, J=4.7, 1.5 Hz, 1H), 12.22 (br s, 1H).

### Reference Example 134

### 1,3-dimethyl-5-(3-methyl-1H-pyrrolo[2,3-b]pyridin-1-yl)-1H-pyrazole-4-carbaldehyde

By a method similar to that in Reference Example 1, the title compound was obtained from 3-methyl-1H-pyrrolo[2,3-b]pyridine and 5-chloro-1,3-dimethyl-1H-pyrazole-4-carbaldehyde.

¹H-NMR (300 MHz, CDCl₃) δ:2.39 (d, J=1.1 Hz, 3H), 2.54 (s, 3H), 3.68 (s, 3H), 6.98-7.11 (m, 1H), 7.21 (dd, J=7.9, 4.7 Hz, 1H), 7.97 (dd, J=7.8, 1.6 Hz, 1H), 8.34 (d, J=1.5 Hz, 1H), 9.58 (s, 1H).

### Reference Example 135

### ethyl (2E)-3-[1,3-dimethyl-5-(3-methyl-1H-pyrrolo[2,3-b]pyridin-1-yl)-1H-pyrazol-4-yl]acrylate

By a method similar to that in Reference Example 12, the title compound was obtained from 1,3-dimethyl-5-(3-methyl-1H-pyrrolo[2,3-b]pyridin-1-yl)-1H-pyrazole-4-carbaldehyde obtained in Reference Example 134 and ethyl (diethoxyphosphoryl)acetate.

¹H-NMR (300 MHz, CDCl₃) δ:1.24 (t, J=7.2 Hz, 3H), 2.39 (d, J=1.1 Hz, 3H), 2.45 (s, 3H), 3.57 (s, 3H), 4.14 (q, J=7.2 Hz, 2H), 5.76 (d, J=16.4 Hz, 1H), 6.96 (d, J=1.1 Hz, 1H), 7.19 (dd, J=7.8, 4.8 Hz, 1H), 7.30 (d, J=16.2 Hz, 1H), 7.96 (dd, J=7.8, 1.6 Hz, 1H), 8.33 (dd, J=4.7, 1.5 Hz, 1H).

### Reference Example 136

### (2E)-3-[1,3-dimethyl-5-(3-methyl-1H-pyrrolo[2,3-b]pyridin-1-yl)-1H-pyrazol-4-yl]acrylic acid

By a method similar to that in Reference Example 13, the title compound was obtained from ethyl (2E)-3-[1,3-dimethyl-5-(3-methyl-1H-pyrrolo[2,3-b]pyridin-1-yl)-1H-pyrazol-4-yl]acrylate obtained in Reference Example 135.

¹H-NMR (300 MHz, DMSO-d₆) δ:2.36 (s, 6H), 3.47 (s, 3H), 5.54 (d, J=16.2 Hz, 1H), 7.01 (d, J=16.4 Hz, 1H), 7.26 (dd, J=7.9, 4.7 Hz, 1H), 7.46 (s, 1H), 8.13 (dd, J=7.9, 1.5 Hz, 1H), 8.26 (dd, J=4.7, 1.5 Hz, 1H), 12.19 (br s, 1H).

### Reference Example 137

### 5-(1H-indol-3-yl)-1,3-dimethyl-1H-pyrazole-4-carbaldehyde

To a mixture of [1-(tert-butoxycarbonyl)-1H-indol-3-yl]boronic acid (2.01 g), 5-chloro-1,3-dimethyl-1H-pyrazole-4-carbaldehyde (2.44 g), a 2.0M aqueous sodium carbonate solution (8.0 mL) and 1,2-dimethoxyethane (16 mL) was added tetrakis(triphenylphosphine)palladium(0) (0.45 g), and the reaction mixture was heated under reflux under nitrogen atmosphere for 12 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 95:5-80:20, v/v) to give a mixture (2.86 g), as a brown solid, of tert-butyl 3-(4-formyl-1,3-dimethyl-1H-pyrazol-5-yl)-1H-indole-1-carboxylate and 5-chloro-1,3-dimethyl-1H-pyrazole-4-carbaldehyde, and the title compound (0.44 g, yield 24%) as a pale-brown solid.

The obtained mixture (2.86 g) of tert-butyl 3-(4-formyl-1,3-dimethyl-1H-pyrazol-5-yl)-1H-indole-1-carboxylate and 5-chloro-1,3-dimethyl-1H-pyrazole-4-carbaldehyde was dissolved in a 4M hydrogen chloride-ethyl acetate solution (45 mL), and the mixture was stirred at room temperature for 3.5 hr. The reaction mixture was diluted with ethyl acetate, washed with water and saturated brine, dried over anhydrous magnesium sulfate, and filtrated. The filtrate was concentrated, and the residue was subjected to silica gel column chromatography (hexane-ethyl acetate 50:50-0:100, v/v) to give the title compound (1.01 g, yield 54%) as a pale-brown solid.

¹H-NMR (300 MHz, CDCl₃) δ:2.57 (s, 3H), 3.76 (s, 3H), 7.03-7.73 (m, 5H), 8.65 (br s, 1H), 9.66 (s, 1H).

### Reference Example 138

### 1,3-dimethyl-5-(1-methyl-1H-indol-3-yl)-1H-pyrazole-4-carbaldehyde

To a solution of 5-(1H-indol-3-yl)-1,3-dimethyl-1H-pyrazole-4-carbaldehyde obtained in Reference Example 137 (1.42 g) in N,N-dimethylformamide (30 mL) was added 60% sodium hydride (in oil, 285 mg) with stirring, and the mixture was stirred at 0° C. for 30 min. Methyl iodide (0.58 mL) was added to this reaction mixture, and the mixture was stirred at room temperature for 5 hr. The reaction mixture was neutralized with 1N hydrochloric acid, and extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 90:10-65:35, v/v), and crystallized from hexane-ethyl acetate to give the title compound (1.15 g, yield 77%) as pale-brown crystals.

¹H-NMR (300 MHz, CDCl₃) δ:2.56 (s, 3H), 3.76 (s, 3H), 3.92 (s, 3H), 7.07-7.57 (m, 5H), 9.65 (s, 1H).

### Reference Example 139

### ethyl (2E)-3-[1,3-dimethyl-5-(1-methyl-1H-indol-3-yl)-1H-pyrazol-4-yl]acrylate

By a method similar to that in Reference Example 12, the title compound was obtained from 1,3-dimethyl-5-(1-methyl-1H-indol-3-yl)-1H-pyrazole-4-carbaldehyde obtained in Reference Example 138 and ethyl (diethoxyphosphoryl)acetate.

¹H-NMR (300 MHz, CDCl₃) δ:1.25 (t, J=7.2 Hz, 3H), 2.49 (s, 3H), 3.70 (s, 3H), 3.90 (s, 3H), 4.15 (q, J=7.0 Hz, 2H), 6.04 (d, J=16.2 Hz, 1H), 7.07-7.66 (m, 6H).

### Reference Example 140

### (2E)-3-[1,3-dimethyl-5-(1-methyl-1H-indol-3-yl)-1H-pyrazol-4-yl]acrylic acid

By a method similar to that in Reference Example 13, the title compound was obtained from ethyl (2E)-3-[1,3-dimethyl-5-(1-methyl-1H-indol-3-yl)-1H-pyrazol-4-yl]acrylate obtained in Reference Example 139.

¹H-NMR (300 MHz, DMSO-d₆) δ:2.36 (s, 3H), 3.63 (s, 3H), 3.92 (s, 3H), 5.88 (d, J=16.2 Hz, 1H), 7.05-7.16 (m, 1H), 7.20-7.36 (m, 3H), 7.59 (d, J=8.9 Hz, 1H), 7.67 (s, 1H), 11.91 (br s, 1H).

### Reference Example 141

### 1,3-dimethyl-5-(3-methyl-1H-indazol-1-yl)-1H-pyrazole-4-carbaldehyde

By a method similar to that in Reference Example 39, the title compound was obtained from 3-methyl-1H-indazole and 5-chloro-1,3-dimethyl-1H-pyrazole-4-carbaldehyde.

¹H-NMR (300 MHz, CDCl₃) δ:2.56 (s, 3H), 2.67 (s, 3H), 3.71 (s, 3H), 6.87-7.88 (m, 4H), 9.58 (s, 1H).

### Reference Example 142

### ethyl (2E)-3-[1,3-dimethyl-5-(3-methyl-1H-indazol-1-yl)-1H-pyrazol-4-yl]acrylate

By a method similar to that in Reference Example 12, the title compound was obtained from 1,3-dimethyl-5-(3-methyl-1H-indazol-1-yl)-1H-pyrazole-4-carbaldehyde obtained in Reference Example 141 and ethyl (diethoxyphosphoryl)acetate.

¹H-NMR (300 MHz, CDCl₃) δ:1.21 (t, J=7.2 Hz, 3H), 2.47 (s, 3H), 2.67 (s, 3H), 3.57 (s, 3H), 4.12 (q, J=7.2 Hz, 2H), 5.72 (d, J=16.4 Hz, 1H), 7.10 (d, J=8.3 Hz, 1H), 7.21-7.35 (m, 2H), 7.37-7.48 (m, 1H), 7.77 (d, J=7.9 Hz, 1H).

### Reference Example 143

### (2E)-3-[1,3-dimethyl-5-(3-methyl-1H-indazol-1-yl)-1H-pyrazol-4-yl]acrylic acid

By a method similar to that in Reference Example 13, the title compound was obtained from ethyl (2E)-3-[1,3-dimethyl-5-(3-methyl-1H-indazol-1-yl)-1H-pyrazol-4-yl]acrylate obtained in Reference Example 142.

¹H-NMR (300 MHz, DMSO-d₆) δ:2.39 (s, 3H), 2.62 (s, 3H), 3.51 (s, 3H), 5.52 (d, J=16.2 Hz, 1H), 7.06 (d, J=16.2 Hz, 1H), 7.20 (d, J=8.5 Hz, 1H), 7.27-7.37 (m, 1H), 7.44-7.57 (m, 1H), 7.93 (d, J=8.1 Hz, 1H), 12.17 (br s, 1H).

### Reference Example 144

### ethyl (2E)-3-[5-(6-methoxy-1H-pyrrolo[2,3-b]pyridin-1-yl)-1,3-dimethyl-1H-pyrazol-4-yl]acrylate

To a solution of 1H-pyrrolo[2,3-b]pyridin-6-ol (5.27 g) in acetone (300 mL) were added dimethylsulfuric acid (4.10 mL) and potassium carbonate (5.50 g), and the mixture was stirred at room temperature for 13 hr. The reaction mixture was filtrated through Celite®, and the filtrate was concentrated. The obtained residue was dissolved in N,N-dimethylformamide (80 mL), 5-chloro-1,3-dimethyl-1H-pyrazole-4-carbaldehyde (7.47 g) and 60% sodium hydride (in oil, 2.36 g) were added with stirring, and the mixture was stirred at 80° C. for 15 hr. After the reaction mixture was allowed to cool to room temperature, water was added, and the mixture was extracted with ethyl acetate. The organic layer was washed with water and saturated brine, dried over anhydrous magnesium sulfate, and filtrated. The filtrate was concentrated, and the residue was subjected to silica gel column chromatography (hexane-ethyl acetate 90:10-75:25, v/v) to give a brown solid (7.53 g).

To a solution of the obtained brown solid (7.53 g) in ethanol (60 mL) were added ethyl (diethoxyphosphoryl)acetate (9.37 g) and sodium ethoxide (3.79 g), and the mixture was stirred at room temperature for 3 hr. Water was added to the reaction mixture, and the mixture was extracted with ethyl acetate. The organic layer was washed with water and saturated brine, dried over anhydrous magnesium sulfate, and filtrated. The filtrate was concentrated, and the residue was subjected to silica gel column chromatography (hexane-ethyl acetate 90:10-65:35, v/v), and crystallized from hexane-ethyl acetate to give the title compound (1.36 g, yield 14%) as colorless crystals.

¹H-NMR (300 MHz, CDCl₃) δ:1.24 (t, J=7.2 Hz, 3H), 2.47 (s, 3H), 3.64 (s, 3H), 3.83 (s, 3H), 4.15 (q, J=7.1 Hz, 2H), 5.77 (d, J=16.2 Hz, 1H), 6.54-6.76 (m, 2H), 6.98 (d, J=3.6 Hz, 1H), 7.35 (d, J=16.2 Hz, 1H), 7.86 (d, J=8.5 Hz, 1H).

### Reference Example 145

### (2E)-3-[5-(6-methoxy-1H-pyrrolo[2,3-b]pyridin-1-yl)-1,3-dimethyl-1H-pyrazol-4-yl]acrylic acid

By a method similar to that in Reference Example 13, the title compound was obtained from ethyl (2E)-3-[5-(6-methoxy-1H-pyrrolo[2,3-b]pyridin-1-yl)-1,3-dimethyl-1H-pyrazol-4-yl]acrylate obtained in Reference Example 144.

¹H-NMR (300 MHz, DMSO-d₆) δ:2.37 (s, 3H), 3.56 (s, 3H), 3.74 (s, 3H), 5.60 (d, J=16.2 Hz, 1H), 6.50-6.84 (m, 2H), 7.12 (d, J=16.2 Hz, 1H), 7.43 (d, J=3.6 Hz, 1H), 8.04 (d, J=8.5 Hz, 1H), 12.15 (br s, 1H).

### Reference Example 146

### tert-butyl (2E)-3-[5-(5-chloro-1H-indol-1-yl)-1,3-dimethyl-1H-pyrazol-4-yl]acrylate

By a method similar to that in Reference Example 12, the title compound was obtained from 5-(5-chloro-1H-indol-1-yl)-1,3-dimethyl-1H-pyrazole-4-carbaldehyde obtained in Reference Example 37 and tert-butyl (diethoxyphosphoryl)acetate.

¹H-NMR (300 MHz, CDCl₃) δ:1.42 (s, 9H), 2.45 (s, 3H), 3.49 (s, 3H), 5.61 (d, J=16.4 Hz, 1H), 6.73 (dd, J=3.4, 0.8 Hz, 1H), 6.92 (d, J=8.9 Hz, 1H), 7.11 (d, J=3.4 Hz, 1H), 7.17 (d, J=16.2 Hz, 1H), 7.20 (dd, J=8.8, 2.0 Hz, 1H), 7.68 (d, J=1.7 Hz, 1H).

### Reference Example 147

### tert-butyl trans-2-[5-(5-chloro-1H-indol-1-yl)-1,3-dimethyl-1H-pyrazol-4-yl]cyclopropanecarboxylate

To a solution of trimethylsulfoxonium iodide (4.71 g) in dimethylsulfoxide (20 mL) was added 60% sodium hydride (in oil, 856 mg) with stirring at room temperature, and the mixture was stirred at room temperature for 1.5 hr. A solution of tert-butyl (2E)-3-[5-(5-chloro-1H-indol-1-yl)-1,3-dimethyl-1H-pyrazol-4-yl]acrylate obtained in Reference Example 146 (3.97 g) in dimethylsulfoxide (60 mL) was added to this reaction mixture with stirring, and the mixture was stirred at room temperature for 48 hr. A saturated aqueous ammonium chloride solution and water were added to the reaction mixture, and the mixture was extracted with ethyl acetate. The organic layer was washed with water and saturated brine, dried over anhydrous magnesium sulfate, and filtrated, and the filtrate was concentrated. The obtained residue was subjected to silica gel column chromatography (hexane-ethyl acetate 100:0-60:40, v/v) to give the title compound (2.21 g, yield 54%) as a colorless oil.

¹H-NMR (300 MHz, CDCl₃) δ:0.36-0.65 (m, 1H), 0.99-1.22 (m, 2H), 1.26 (s, 5H), 1.33 (s, 4H), 1.86-2.00 (m, 1H), 2.32 (s, 3H), 3.47 (s, 1.5H), 3.49 (s, 1.5H), 6.53-6.72 (m, 1H), 6.92 (d, J=8.7 Hz, 1H), 7.07 (dd, J=11.0, 3.4 Hz, 1H), 7.14-7.22 (m, 1H), 7.66 (d, J=1.9 Hz, 1H).

### Reference Example 148

### trans-2-[5-(5-chloro-1H-indol-1-yl)-1,3-dimethyl-1H-pyrazol-4-yl]cyclopropanecarboxylic acid

tert-Butyl trans-2-[5-(5-chloro-1H-indol-1-yl)-1,3-dimethyl-1H-pyrazol-4-yl]cyclopropanecarboxylate obtained in Reference Example 147 (2.15 g) was dissolved in a 4M hydrogen chloride-ethyl acetate solution (10 mL), and the mixture was stirred at room temperature for 3 hr, and then at 50° C. for 12 hr. The reaction mixture was allowed to cool to room temperature, and diluted with ethyl acetate. The organic layer was washed with saturated brine, dried over anhydrous magnesium sulfate, and filtrated, and the filtrate was concentrated. The obtained residue was crystallized from hexane-ethyl acetate to give the title compound (0.52 g, yield 43%) as colorless crystals (a solvate with 0.05 mol ethyl acetate/mol).

¹H-NMR (300 MHz, DMSO-d₆) δ:0.40-0.60 (m, 1H), 0.71-0.87 (m, 0.5H), 0.88-1.03 (m, 0.5H), 1.11-1.24 (m, 0.5H), 1.25-1.38 (m, 0.5H), 1.80-1.97 (m, 1H), 2.22 (s, 3H), 3.38 (s, 1.5H), 3.38 (s, 1.5H), 6.76 (dd, J=2.3, 1.1 Hz, 1H), 7.04 (t, J=9.3 Hz, 1H), 7.13-7.25 (m, 1H), 7.58 (d, J=3.4 Hz, 1H), 7.74 (t, J=1.9 Hz, 1H), 12.09 (br s, 1H).

### Reference Example 149

### 5-cyclopropyl-2-methyl-2,4-dihydro-3H-pyrazol-3-one

To a solution of methyl 3-cyclopropyl-3-oxopropionoate (39.8 g) in toluene (150 mL) was added methylhydrazine (13.0 g), and the mixture was heated under reflux for 4 hr. The reaction mixture was allowed to cool to room temperature, and concentrated under reduced pressure. The residue was crystallized from diethyl ether to give the title compound (37.9 g, yield 98%) as colorless crystals.

¹H-NMR (300 MHz, CDCl₃) δ:0.74-0.82 (m, 2H), 0.92-1.01 (m, 2H), 1.71-1.83 (m, 1H), 3.05 (s, 2H), 3.26 (s, 3H).

### Reference Example 150

### 5-chloro-3-cyclopropyl-1-methyl-1H-pyrazole-4-carbaldehyde

Phosphoryl chloride (165 g) was added dropwise over 25 min to N,N-dimethylformamide (19.0 g) cooled at 0° C. 5-Cyclopropyl-2-methyl-2,4-dihydro-3H-pyrazol-3-one obtained in Reference Example 149 (29.7 g) was added to this reaction mixture, and the mixture was stirred with heating at 100° C. for 2 hr. The reaction mixture was allowed to cool to room temperature, and poured into ice water, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 85:15, v/v) to give the title compound (39.1 g, yield 98%) as colorless crystals.

¹H-NMR (300 MHz, CDCl₃) δ:0.89-1.03 (m, 4H), 2.39-2.51 (m, 1H), 3.77 (s, 3H), 9.91 (s, 1H).

### Reference Example 151

### benzyl [(ethylamino)sulfonyl]carbamate

By a method similar to that in Reference Example 102, the title compound was obtained from benzyl alcohol, chlorosulfonyl isocyanate and ethanamine.

¹H-NMR (300 MHz, CDCl₃) δ:1.13-1.20 (m, 3H), 3.05-3.16 (m, 2H), 5.17-5.21 (m, 2H), 7.33-7.39 (m, 5H), 7.64 (br s, 1H).

### Reference Example 152

### N-ethylsulfamide

By a method similar to that in Reference Example 109, the title compound was obtained from benzyl [(ethylamino)sulfonyl]carbamate obtained in Reference Example 151.

¹H-NMR (300 MHz, DMSO-d₆) δ:1.03-1.10 (m, 3H), 2.84-2.95 (m, 2H), 6.37-6.49 (m, 3H).

### Reference Example 153

### 3-cyclopropyl-5-(5-fluoro-1H-indol-1-yl)-1-methyl-1H-pyrazole-4-carbaldehyde

By a method similar to that in Reference Example 1, the title compound was obtained from 5-fluoro-1H-indole and 5-chloro-3-cyclopropyl-1-methyl-1H-pyrazole-4-carbaldehyde obtained in Reference Example 150.

¹H-NMR (300 MHz, CDCl₃) δ:0.99-1.11 (m, 4H), 2.50-2.61 (m, 1H), 3.53 (s, 3H), 6.77 (d, J=3.4 Hz, 1H), 7.00-7.04 (m, 2H), 7.22 (d, J=3.2 Hz, 1H), 7.33-7.38 (m, 1H), 9.57 (s, 1H).

### Reference Example 154

### ethyl (2E)-3-[3-cyclopropyl-5-(5-fluoro-1H-indol-1-yl)-1-methyl-1H-pyrazol-4-yl]acrylate

By a method similar to that in Reference Example 12, the title compound was obtained from 3-cyclopropyl-5-(5-fluoro-1H-indol-1-yl)-1-methyl-1H-pyrazole-4-carbaldehyde obtained in Reference Example 153 and ethyl (diethoxyphosphoryl)acetate.

¹H-NMR (300 MHz, CDCl₃) δ:0.92-1.07 (m, 4H), 1.22 (t, J=7.2 Hz, 3H), 1.94-2.06 (m, 1H), 3.47 (s, 3H), 4.13 (q, J=7.1 Hz, 2H), 5.79 (d, J=16.3 Hz, 1H), 6.75 (d, J=3.4 Hz, 1H), 6.88-7.03 (m, 2H), 7.12 (d, J=3.4 Hz, 1H), 7.33-7.43 (m, 2H).

### Reference Example 155

### (2E)-3-[3-cyclopropyl-5-(5-fluoro-1H-indol-1-yl)-1-methyl-1H-pyrazol-4-yl]acrylic acid

By a method similar to that in Reference Example 13, the title compound was obtained from ethyl (2E)-3-[3-cyclopropyl-5-(5-fluoro-1H-indol-1-yl)-1-methyl-1H-pyrazol-4-yl]acrylate obtained in Reference Example 154.

¹H-NMR (300 MHz, DMSO-d₆) δ:0.77-1.02 (m, 4H), 1.99-2.12 (m, 1H), 3.46 (s, 3H), 5.49 (d, J=16.3 Hz, 1H), 6.85 (d, J=3.4 Hz, 1H), 6.99-7.11 (m, 2H), 7.15-7.22 (m, 1H), 7.52 (dd, J=9.5, 1.9 Hz, 1H), 7.67 (d, J=3.0 Hz, 1H), 12.15 (br s, 1H).

### Reference Example 156

### ethyl (2E)-3-[5-(6-hydroxy-1H-indol-1-yl)-1,3-dimethyl-1H-pyrazol-4-yl]acrylate

To a solution of ethyl (2E)-3-{5-[6-(benzyloxy)-1H-indol-1-yl]-1,3-dimethyl-1H-pyrazol-4-yl}acrylate obtained in Reference Example 27 (31.9 g) in dichloromethane (150 mL) was added dropwise boron tribromide (1M dichloromethane solution, 154 mL) with stirring at −78° C., and the mixture was stirred at −78° C. for 3 hr. The reaction mixture was quenched with ethanol (100 mL), and concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v) to give the title compound (18.9 g, yield 76%) as a colorless amorphous solid.

¹H-NMR (300 MHz, CDCl₃) δ:1.23 (t, J=7.2 Hz, 3H), 2.41 (s, 3H), 3.48 (s, 3H), 4.13 (q, J=7.2 Hz, 2H), 5.64 (d, J=16.2 Hz, 1H), 6.32 (d, J=2.1 Hz, 1H), 6.69-6.73 (m, 1H), 6.83 (dd, J=8.6, 2.2 Hz, 1H), 6.94 (d, J=3.2 Hz, 1H), 7.19 (s, 1H), 7.30 (d, J=16.4 Hz, 1H), 7.54 (d, J=8.5 Hz, 1H).

### Reference Example 157

### ethyl (2E)-3-{1,3-dimethyl-5-[6-(2-oxopropoxy)-1H-indol-1-yl]-1H-pyrazol-4-yl}acrylate

To a solution of ethyl (2E)-3-[5-(6-hydroxy-1H-indol-1-yl)-1,3-dimethyl-1H-pyrazol-4-yl]acrylate obtained in Reference Example 156 (2.02 g) in acetone (10 mL) were added chloroacetone (689 mg), potassium carbonate (1.28 g) and sodium iodide (1.28 g), and the mixture was stirred at 50° C. for 16 hr. The reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (2.26 g, yield 95%) as colorless crystals.

¹H-NMR (300 MHz, CDCl₃) δ:1.18-1.25 (m, 3H), 2.27 (s, 3H), 2.47 (s, 3H), 3.48-3.52 (m, 3H), 4.12 (q, J=7.0 Hz, 2H), 4.49 (s, 2H), 5.55-5.68 (m, 1H), 6.42 (d, J=1.7 Hz, 1H), 6.73 (d, J=3.2 Hz, 1H), 6.90 (dd, J=8.7, 2.3 Hz, 1H), 7.00 (d, J=3.4 Hz, 1H), 7.25-7.34 (m, 1H), 7.58-7.64 (m, 1H).

### Reference Example 158

### ethyl (2E)-3-{5-[6-(2-methoxyethoxy)-1H-indol-1-yl]-1,3-dimethyl-1H-pyrazol-4-yl}acrylate

To a solution of ethyl (2E)-3-[5-(6-hydroxy-1H-indol-1-yl)-1,3-dimethyl-1H-pyrazol-4-yl]acrylate obtained in Reference Example 156 (1.08 g) in N,N-dimethylformamide (5 mL) were added bromoethyl methyl ether (553 mg), potassium carbonate (688 mg) and sodium iodide (995 mg), and the mixture was stirred at 80° C. for 16 hr. The reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v), and crystallized from hexane-ethyl acetate to give the title compound (1.26 g, yield 99%) as colorless crystals.

¹H-NMR (300 MHz, CDCl₃) δ:1.16-1.25 (m, 3H), 2.46 (s, 3H), 3.43 (s, 3H), 3.48-3.52 (m, 3H), 3.70-3.77 (m, 2H), 4.03-4.17 (m, 4H), 5.55-5.68 (m, 1H), 6.41-6.48 (m, 1H), 6.71 (d, J=3.4 Hz, 1H), 6.89-6.98 (m, 2H), 7.28-7.35 (m, 1H), 7.57 (d, J=8.7 Hz, 1H).

### Reference Example 159

### (2E)-3-{5-[6-(2-methoxyethoxy)-1H-indol-1-yl]-1,3-dimethyl-1H-pyrazol-4-yl}acrylic acid

By a method similar to that in Reference Example 13, the title compound was obtained from ethyl (2E)-3-{5-[6-(2-methoxyethoxy)-1H-indol-1-yl]-1,3-dimethyl-1H-pyrazol-4-yl}acrylate obtained in Reference Example 158.

¹H-NMR (300 MHz DMSO-d₆) δ:2.38 (s, 3H), 3.27 (s, 3H), 3.49 (s, 3H), 3.61 (t, J=4.5 Hz, 2H), 3.92-4.11 (m, 2H), 5.38-5.48 (m, 1H), 6.50 (d, J=1.7 Hz, 1H), 6.76 (d, J=3.2 Hz, 1H), 6.84 (dd, J=8.7, 2.1 Hz, 1H), 7.09 (d, J=16.2 Hz, 1H), 7.39 (d, J=3.4 Hz, 1H), 7.58 (d, J=8.7 Hz, 1H), 12.15 (br s, 1H).

### Reference Example 160

### ethyl (2E)-3-{5-[6-(cyclopropylmethoxy)-1H-indol-1-yl]-1,3-dimethyl-1H-pyrazol-4-yl}acrylate

By a method similar to that in Reference Example 157, the title compound was obtained from ethyl (2E)-3-[5-(6-hydroxy-1H-indol-1-yl)-1,3-dimethyl-1H-pyrazol-4-yl]acrylate obtained in Reference Example 156 and bromomethylcyclopropane.

¹H-NMR (300 MHz, CDCl₃) δ:0.29-0.36 (m, 2H), 0.58-0.66 (m, 2H), 1.17-1.29 (m, 4H), 2.47 (s, 3H), 3.49 (s, 3H), 3.73 (d, J=6.8 Hz, 2H), 4.12 (q, J=7.1 Hz, 2H), 5.64 (d, J=16.2 Hz, 1H), 6.41 (d, J=2.1 Hz, 1H), 6.71 (dd, J=3.4, 0.8 Hz, 1H), 6.90 (dd, J=8.7, 2.3 Hz, 1H), 6.96 (d, J=3.4 Hz, 1H), 7.31 (d, J=16.2 Hz, 1H), 7.56 (d, J=8.7 Hz, 1H).

### Reference Example 161

### (2E)-3-{5-[6-(cyclopropylmethoxy)-1H-indol-1-yl]-1,3-dimethyl-1H-pyrazol-4-yl}acrylic acid

By a method similar to that in Reference Example 13, the title compound was obtained from ethyl (2E)-3-{5-[6-(cyclopropylmethoxy)-1H-indol-1-yl]-1,3-dimethyl-1H-pyrazol-4-yl}acrylate obtained in Reference Example 160.

¹H-NMR (300 MHz, DMSO-d₆) δ:0.24-0.32 (m, 2H), 0.47-0.56 (m, 2H), 1.12-1.21 (m, 1H), 2.38 (s, 3H), 3.48 (s, 3H), 3.66-3.80 (m, 2H), 5.42 (d, J=16.2 Hz, 1H), 6.45 (d, J=1.7 Hz, 1H), 6.75 (d, J=3.0 Hz, 1H), 6.84 (dd, J=8.7, 2.3 Hz, 1H), 7.08 (d, J=16.2 Hz, 1H), 7.37 (d, J=3.4 Hz, 1H), 7.57 (d, J=8.7 Hz, 1H), 12.13 (br s, 1H).

### Reference Example 162

### ethyl (2E)-3-[5-(6-isopropoxy-1H-indol-1-yl)-1,3-dimethyl-1H-pyrazol-4-yl]acrylate

To a solution of ethyl (2E)-3-[5-(6-hydroxy-1H-indol-1-yl)-1,3-dimethyl-1H-pyrazol-4-yl]acrylate obtained in Reference Example 156 (1.54 g) in tetrahydrofuran (20 mL) were added isopropanol (426 mg) and tributylphosphine (1.91 g), 1,1′-azodicarbonyldipiperidine (2.38 g) was added with stirring, and the mixture was stirred at room temperature for 1 hr. The reaction mixture was concentrated under reduced pressure, diisopropyl ether was added to the residue, and the insoluble material was filtered off. The filtrate was concentrated, and the residue was subjected to silica gel column chromatography (hexane-ethyl acetate 70:30, v/v) to give the title compound (1.66 g, yield 96%) as a colorless oil.

¹H-NMR (300 MHz, CDCl₃) δ:1.19-1.24 (m, 3H), 1.29 (dd, J=6.0, 3.6 Hz, 6H), 2.46 (s, 3H), 3.51 (s, 3H), 4.12 (q, J=7.2 Hz, 2H), 4.42-4.52 (m, 1H), 5.66 (d, J=16.2 Hz, 1H), 6.46 (d, J=2.1 Hz, 1H), 6.68-6.71 (m, 1H), 6.86 (dd, J=8.6, 2.2 Hz, 1H), 6.96 (d, J=3.4 Hz, 1H), 7.32 (d, J=16.2 Hz, 1H), 7.56 (d, J=8.5 Hz, 1H).

### Reference Example 163

### (2E)-3-[5-(6-isopropoxy-1H-indol-1-yl)-1,3-dimethyl-1H-pyrazol-4-yl]acrylic acid

By a method similar to that in Reference Example 13, the title compound was obtained from ethyl (2E)-3-[5-(6-isopropoxy-1H-indol-1-yl)-1,3-dimethyl-1H-pyrazol-4-yl]acrylate obtained in Reference Example 162.

¹H-NMR (300 MHz, DMSO-d₆) δ:1.20 (dd, J=9.5, 5.9 Hz, 6H), 2.38 (s, 3H), 3.49 (s, 3H), 4.46-4.56 (m, 1H), 5.41-5.49 (m, 1H), 6.47 (d, J=1.7 Hz, 1H), 6.74 (d, J=2.8 Hz, 1H), 6.81 (dd, J=8.7, 2.1 Hz, 1H), 7.08 (d, J=16.2 Hz, 1H), 7.40 (d, J=3.2 Hz, 1H), 7.57 (d, J=8.5 Hz, 1H), 12.19 (br s, 1H).

### Reference Example 164

### benzyl {[(allyloxy)amino]sulfonyl}carbamate

By a method similar to that in Reference Example 102, the title compound was obtained from benzyl alcohol, chlorosulfonyl isocyanate and O-allylhydroxylamine hydrochloride.

¹H-NMR (300 MHz, CDCl₃) δ:4.47 (d, J=6.4 Hz, 2H), 5.22 (s, 2H), 5.27-5.39 (m, 2H), 5.85-6.00 (m, 1H), 7.31-7.42 (m, 5H), 7.50 (br s, 1H), 7.83 (s, 1H).

### Reference Example 165

### N-propoxysulfamide

By a method similar to that in Reference Example 109, the title compound was obtained from benzyl {[(allyloxy)amino]sulfonyl}carbamate obtained in Reference Example 164.

¹H-NMR (300 MHz, CDCl₃) δ:0.94 (t, J=7.4 Hz, 3H), 1.60-1.74 (m, 2H), 3.96 (t, J=6.8 Hz, 2H), 5.17 (br s, 2H).

### Reference Example 166

### ethyl (2E)-3-[5-(6-ethoxy-1H-indol-1-yl)-1,3-dimethyl-1H-pyrazol-4-yl]acrylate

By a method similar to that in Reference Example 162, the title compound was obtained from ethyl (2E)-3-[5-(6-hydroxy-1H-indol-1-yl)-1,3-dimethyl-1H-pyrazol-4-yl]acrylate obtained in Reference Example 156 and ethanol.

¹H-NMR (300 MHz, CDCl₃) δ:1.22 (t, J=7.1 Hz, 3H), 1.39 (t, J=7.0 Hz, 3H), 2.46 (s, 3H), 3.50 (s, 3H), 3.97 (q, J=7.0 Hz, 2H), 4.12 (q, J=7.1 Hz, 2H), 5.64 (d, J=16.4 Hz, 1H), 6.43 (d, J=1.7 Hz, 1H), 6.71 (d, J=3.2 Hz, 1H), 6.87 (dd, J=8.7, 2.3 Hz, 1H), 6.96 (d, J=3.4 Hz, 1H), 7.32 (d, J=16.2 Hz, 1H), 7.56 (d, J=8.7 Hz, 1H).

### Reference Example 167

### (2E)-3-[5-(6-ethoxy-1H-indol-1-yl)-1,3-dimethyl-1H-pyrazol-4-yl]acrylic acid

By a method similar to that in Reference Example 13, the title compound was obtained from ethyl (2E)-3-[5-(6-ethoxy-1H-indol-1-yl)-1,3-dimethyl-1H-pyrazol-4-yl]acrylate obtained in Reference Example 166.

¹H-NMR (300 MHz, DMSO-d₆) δ:1.28 (t, J=7.0 Hz, 3H), 2.38 (s, 3H), 3.49 (s, 3H), 3.84-4.02 (m, 2H), 5.38-5.47 (m, 1H), 6.46 (s, 1H), 6.75 (d, J=3.2 Hz, 1H), 6.82 (dd, J=8.7, 2.1 Hz, 1H), 7.09 (d, J=16.2 Hz, 1H), 7.38 (d, J=3.2 Hz, 1H), 7.58 (d, J=8.7 Hz, 1H), 12.14 (br s, 1H).

### Reference Example 168

### ethyl (2E)-3-{5-[6-(2-tert-butoxy-1-methyl-2-oxoethoxy)-1H-indol-1-yl]-1,3-dimethyl-1H-pyrazol-4-yl}acrylate

By a method similar to that in Reference Example 158, the title compound was obtained from ethyl (2E)-3-[5-(6-hydroxy-1H-indol-1-yl)-1,3-dimethyl-1H-pyrazol-4-yl]acrylate obtained in Reference Example 156 and tert-butyl 2-bromopropionoate.

¹H-NMR (300 MHz, CDCl₃) δ:1.22 (t, J=6.6 Hz, 3H), 1.35 (d, J=3.8 Hz, 9H), 1.53-1.60 (m, 3H), 2.45 (d, J=3.4 Hz, 3H), 3.44-3.50 (m, 3H), 4.07-4.17 (m, 2H), 4.49-4.59 (m, 1H), 5.61 (dd, J=16.3, 8.0 Hz, 1H), 6.40 (dd, J=8.5, 2.1 Hz, 1H), 6.71 (d, J=2.7 Hz, 1H), 6.84-6.92 (m, 1H), 6.97 (d, J=3.4 Hz, 1H), 7.24-7.35 (m, 1H), 7.57 (d, J=8.3 Hz, 1H).

### Reference Example 169

### 2-[(1-{4-[(1E)-3-ethoxy-3-oxoprop-1-en-1-yl]-1,3-dimethyl-1H-pyrazol-5-yl}-1H-indol-6-yl)oxy]propanoic acid

A solution of ethyl (2E)-3-{5-[6-(2-tert-butoxy-1-methyl-2-oxoethoxy)-1H-indol-1-yl]-1,3-dimethyl-1H-pyrazol-4-yl}acrylate obtained in Reference Example 168 (2.51 g) in trifluoroacetic acid (20 mL) was stirred at room temperature for 1 hr. The reaction mixture was concentrated under reduced pressure, water was added, and the mixture was extracted with ethyl acetate. The organic layer was washed with saturated brine, dried over anhydrous magnesium sulfate, and filtrated. The filtrate was concentrated, and the residue was crystallized from hexane-ethanol to give the title compound (1.81 g, yield 83%) as colorless crystals.

¹H-NMR (300 MHz, DMSO-d₆) δ:1.13 (t, J=7.1 Hz, 3H), 1.44 (dd, J=6.8, 1.5 Hz, 3H), 2.39 (s, 3H), 3.46 (d, J=7.9 Hz, 3H), 3.99-4.09 (m, 2H), 4.71-4.86 (m, 1H), 5.49 (dd, J=20.5, 16.2 Hz, 1H), 6.45 (dd, J=8.9, 1.8 Hz, 1H), 6.77 (d, J=3.4 Hz, 1H), 6.83 (dd, J=8.6, 2.2 Hz, 1H), 7.15 (dd, J=16.3, 6.7 Hz, 1H), 7.40 (d, J=3.4 Hz, 1H), 7.60 (d, J=8.7 Hz, 1H), 12.95 (br s, 1H).

### Reference Example 170

### ethyl (2E)-3-{5-[6-(2-hydroxy-1-methylethoxy)-1H-indol-1-yl]-1,3-dimethyl-1H-pyrazol-4-yl}acrylate

To a solution of 2-[(1-{4-[(1E)-3-ethoxy-3-oxoprop-1-en-1-yl]-1,3-dimethyl-1H-pyrazol-5-yl}-1H-indol-6-yl)oxy]propanoic acid obtained in Reference Example 169 (201 mg) and N,N-dimethylformamide (0.1 mL) in tetrahydrofuran (5 mL) was added dropwise oxalyl chloride (96.3 mg), and the mixture was stirred at room temperature for 1 hr. The reaction mixture was concentrated under reduced pressure, and tetrahydrofuran (5 mL) and water (0.5 mL) were added to the residue. Sodium borohydride (28.7 mg) was added, and the mixture was stirred at room temperature for 18 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 35:65, v/v) to give the title compound (97.3 mg, yield 50%) as a colorless amorphous solid.

¹H-NMR (300 MHz, CDCl₃) δ:1.18-1.26 (m, 6H), 2.47 (s, 3H), 3.52 (s, 3H), 3.63-3.76 (m, 2H), 4.12 (q, J=7.1 Hz, 2H), 4.38-4.47 (m, 1H), 5.66 (dd, J=16.3, 1.5 Hz, 1H), 6.52 (d, J=1.9 Hz, 1H), 6.72 (d, J=3.0 Hz, 1H), 6.87-6.93 (m, 1H), 6.99 (d, J=3.4 Hz, 1H), 7.31 (dd, J=16.3, 2.7 Hz, 1H), 7.58 (d, J=8.7 Hz, 1H).

### Reference Example 171

### ethyl (2E)-3-{5-[6-(2-methoxy-1-methylethoxy)-1H-indol-1-yl]-1,3-dimethyl-1H-pyrazol-4-yl}acrylate

To a solution of ethyl (2E)-3-{5-[6-(2-hydroxy-1-methylethoxy)-1H-indol-1-yl]-1,3-dimethyl-1H-pyrazol-4-yl}acrylate obtained in Reference Example 170 (656 mg) in N,N-dimethylformamide (2 mL) was added 60% sodium hydride (in oil, 102 mg) with stirring, and the mixture was stirred at 0° C. for 30 min. Methyl iodide (0.16 mL) was added to this reaction mixture, and the mixture was stirred at room temperature for 48 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 65:35, v/v) to give the title compound (496 mg, yield 73%) as colorless crystals.

¹H-NMR (300 MHz, CDCl₃) δ:1.17-1.30 (m, 6H), 2.46 (s, 3H), 3.38 (d, J=1.5 Hz, 3H), 3.42-3.59 (m, 5H), 4.12 (q, J=7.2 Hz, 2H), 4.41-4.52 (m, 1H), 5.57-5.69 (m, 1H), 6.50-6.54 (m, 1H), 6.71 (d, J=3.4 Hz, 1H), 6.88-6.94 (m, 1H), 6.95-6.99 (m, 1H), 7.28-7.36 (m, 1H), 7.53-7.60 (m, 1H).

### Reference Example 172

### (2E)-3-{5-[6-(2-methoxy-1-methylethoxy)-1H-indol-1-yl]-1,3-dimethyl-1H-pyrazol-4-yl}acrylic acid

By a method similar to that in Reference Example 13, the title compound was obtained from ethyl (2E)-3-{5-[6-(2-methoxy-1-methylethoxy)-1H-indol-1-yl]-1,3-dimethyl-1H-pyrazol-4-yl}acrylate obtained in Reference Example 171.

¹H-NMR (300 MHz, DMSO-d₆) δ:1.15 (dd, J=11.1, 6.2 Hz, 3H), 2.38 (s, 3H), 3.24 (d, J=7.0 Hz, 3H), 3.37-3.46 (m, 2H), 3.49 (s, 3H), 4.47-4.57 (m, 1H), 5.44 (dd, J=16.2, 4.3 Hz, 1H), 6.53 (s, 1H), 6.75 (d, J=3.2 Hz, 1H), 6.83 (dd, J=8.7, 2.1 Hz, 1H), 7.05-7.13 (m, 1H), 7.41 (dd, J=3.4, 2.1 Hz, 1H), 7.55-7.60 (m, 1H), 12.17 (br s, 1H).

### Reference Example 173

### 1,3-dimethyl-5-(5-methyl-1H-indol-1-yl)-1H-pyrazole-4-carbaldehyde

By a method similar to that in Reference Example 1, the title compound was obtained from 5-methyl-1H-indole and 5-chloro-1,3-dimethyl-1H-pyrazole-4-carbaldehyde.

¹H-NMR (300 MHz, CDCl₃) δ:2.47 (s, 3H), 2.55 (s, 3H), 3.57 (s, 3H), 6.72 (d, J=3.4 Hz, 1H), 6.98-7.02 (m, 1H), 7.08-7.13 (m, 1H), 7.15 (d, J=3.0 Hz, 1H), 7.49 (s, 1H), 9.51 (s, 1H).

### Reference Example 174

### ethyl (2E)-3-[1,3-dimethyl-5-(5-methyl-1H-indol-1-yl)-1H-pyrazol-4-yl]acrylate

By a method similar to that in Reference Example 12, the title compound was obtained from 1,3-dimethyl-5-(5-methyl-1H-indol-1-yl)-1H-pyrazole-4-carbaldehyde obtained in Reference Example 173 and ethyl (diethoxyphosphoryl)acetate.

¹H-NMR (300 MHz, CDCl₃) δ:1.22 (t, J=7.2 Hz, 3H), 2.44-2.49 (m, 6H), 3.49 (s, 3H), 4.12 (q, J=7.2 Hz, 2H), 5.64 (d, J=16.2 Hz, 1H), 6.71 (dd, J=3.4, 0.8 Hz, 1H), 6.89 (d, J=8.3 Hz, 1H), 7.03-7.09 (m, 2H), 7.30 (d, J=16.4 Hz, 1H), 7.50 (s, 1H).

### Reference Example 175

### (2E)-3-[1,3-dimethyl-5-(5-methyl-1H-indol-1-yl)-1H-pyrazol-4-yl]acrylic acid

By a method similar to that in Reference Example 13, the title compound was obtained from ethyl (2E)-3-[1,3-dimethyl-5-(5-methyl-1H-indol-1-yl)-1H-pyrazol-4-yl]acrylate obtained in Reference Example 174.

¹H-NMR (300 MHz, DMSO-d₆) δ:2.35-2.42 (m, 6H), 3.47 (s, 3H), 5.41 (d, J=16.4 Hz, 1H), 6.76 (d, J=3.2 Hz, 1H), 6.90 (1H, d, J=8.3 Hz), 7.01-7.10 (m, 2H), 7.47-7.52 (m, 2H), 12.15 (br s, 1H).

### Reference Example 176

### benzyl (piperidin-1-ylsulfonyl)carbamate

By a method similar to that in Reference Example 102, the title compound was obtained from benzyl alcohol, chlorosulfonyl isocyanate and piperidine.

¹H-NMR (300 MHz, CDCl₃) δ:1.46-1.69 (m, 6H), 3.27-3.35 (m, 4H), 5.17 (s, 2H), 7.37 (s, 5H).

### Reference Example 177

### piperidine-1-sulfonamide

By a method similar to that in Reference Example 109, the title compound was obtained from benzyl (piperidin-1-ylsulfonyl)carbamate obtained in Reference Example 176.

¹H-NMR (300 MHz, CDCl₃) δ:1.47-1.58 (m, 2H), 1.63-1.73 (m, 4H), 3.11-3.16 (m, 4H), 4.79 (br s, 2H).

### Reference Example 178

### (E)-2-[5-(5-chloro-1H-indol-1-yl)-1,3-dimethyl-1H-pyrazol-4-yl]ethylenesulfonamide

To a solution of tert-butyl {[(diphenylphosphoryl)methyl]sulfonyl}carbamate (3.46 g) in N,N-dimethylformamide (73 mL) was added 60% sodium hydride (in oil, 876 mg) with stirring at 0° C., and the mixture was stirred at 0° C. for 1 hr. 5-(5-Chloro-1H-indol-1-yl)-1,3-dimethyl-1H-pyrazole-4-carbaldehyde obtained in Reference Example 37 (2.0 g) was added to the reaction mixture, and the mixture was stirred at room temperature for 1 hr. A saturated aqueous ammonium chloride solution (30 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 55:45, v/v) to give a colorless oil. Trifluoroacetic acid (15 mL) was added to this colorless oil, and the mixture was stirred at room temperature for 3 hr. The reaction mixture was concentrated under reduced pressure, water was added, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (2.52 g, yield 95%) as colorless crystals.

¹H-NMR (300 MHz, CDCl₃) δ:2.42 (s, 3H), 3.53 (s, 3H), 4.48 (s, 2H), 5.88 (d, J=15.5 Hz, 1H), 6.77 (d, J=3.4 Hz, 1H), 6.91 (d, J=8.7 Hz, 1H), 7.09-7.17 (m, 2H), 7.22 (dd, J=8.7, 1.9 Hz, 1H), 7.69 (d, J=1.9 Hz, 1H).

### Reference Example 179

### 2-[5-(5-chloro-1H-indol-1-yl)-1,3-dimethyl-1H-pyrazol-4-yl]ethanesulfonamide

To a solution of (E)-2-[5-(5-chloro-1H-indol-1-yl)-1,3-dimethyl-1H-pyrazol-4-yl]ethylenesulfonamide obtained in Reference Example 178 (2.18 g) in a mixed solvent of tetrahydrofuran (31 mL) and ethanol (31 mL) was added 10% palladium carbon (218 mg), and the mixture was stirred under 1 atom of hydrogen atmosphere at room temperature for 5 hr. The catalyst was removed by filtration, and the filtrate was concentrated. The residue was subjected to silica gel column chromatography (hexane-ethyl acetate 40:60, v/v), and crystallized from hexane-ethyl acetate to give the title compound (1.65 g, yield 75%) as colorless crystals.

¹H-NMR (300 MHz, CDCl₃) δ:2.31 (s, 3H), 2.71-2.82 (m, 2H), 2.87-2.97 (m, 2H), 3.47 (s, 3H), 4.43 (br s, 2H), 6.70 (d, J=2.8 Hz, 1H), 6.92 (d, J=8.7 Hz, 1H), 7.11 (d, J=3.2 Hz, 1H), 7.20 (dd, J=8.7, 1.9 Hz, 1H), 7.67 (d, J=1.9 Hz, 1H).

### Reference Example 180

### (E)-2-[5-(6-chloro-1H-indol-1-yl)-1,3-dimethyl-1H-pyrazol-4-yl]ethylenesulfonamide

By a method similar to that in Reference Example 178, the title compound was obtained from 5-(6-chloro-1H-indol-1-yl)-1,3-dimethyl-1H-pyrazole-4-carbaldehyde obtained in Reference Example 24 and tert-butyl {[(diphenylphosphoryl)methyl]sulfonyl}carbamate.

¹H-NMR (300 MHz, CDCl₃) δ:2.44 (s, 3H), 3.55 (s, 3H), 4.46 (br s, 2H), 5.87 (d, J=15.6 Hz, 1H), 6.80 (dd, J=3.3, 0.8 Hz, 1H), 6.97-6.99 (m, 1H), 7.08 (d, J=3.4 Hz, 1H), 7.15 (d, J=15.6 Hz, 1H), 7.22 (dd, J=8.5, 1.7 Hz, 1H), 7.63 (d, J=8.5 Hz, 1H).

### Reference Example 181

### 2-[5-(6-chloro-1H-indol-1-yl)-1,3-dimethyl-1H-pyrazol-4-yl]ethanesulfonamide

By a method similar to that in Reference Example 179, the title compound was obtained from (E)-2-[5-(6-chloro-1H-indol-1-yl)-1,3-dimethyl-1H-pyrazol-4-yl]ethylenesulfonamide obtained in Reference Example 180.

¹H-NMR (300 MHz, DMSO-d₆) δ:2.22 (s, 3H), 2.44-2.56 (m, 1H), 2.60-2.72 (m, 1H), 2.75-2.92 (m, 2H), 3.37 (s, 3H), 6.77 (s, 2H), 6.80-6.83 (m, 1H), 7.04-7.07 (m, 1H), 7.20 (dd, J=8.5, 1.9 Hz, 1H), 7.57 (d, J=3.4 Hz, 1H), 7.71 (d, J=8.5 Hz, 1H).

### Reference Example 182

### 5-(5-chloro-1H-pyrrolo[2,3-b]pyridin-1-yl)-1,3-dimethyl-1H-pyrazole-4-carbaldehyde

To a solution of 5-chloro-1H-pyrrolo[2,3-b]pyridine (2.91 g) in N,N-dimethylformamide (25 mL), which was cooled at 0° C. in an ice bath, was added 60% sodium hydride (in oil, 970 mg) with stirring, and the mixture was stirred at 0° C. for 30 min. 5-Chloro-1,3-dimethyl-1H-pyrazole-4-carbaldehyde (2.93 g) was added to this reaction mixture at 0° C., and the reaction mixture was stirred at 80° C. for 30 min. After the reaction mixture was allowed to cool to room temperature, water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 60:40, v/v) to give the title compound (1.55 g, yield 30%) as colorless crystals.

¹H-NMR (300 MHz, CDCl₃) δ:2.55 (s, 3H), 3.67 (s, 3H), 6.74 (d, J=3.6 Hz, 1H), 7.36 (d, J=3.6 Hz, 1H), 8.00 (d, J=2.3 Hz, 1H), 8.29 (d, J=2.3 Hz, 1H), 9.60 (s, 1H).

### Reference Example 183

### (E)-2-[5-(5-chloro-1H-pyrrolo[2,3-b]pyridin-1-yl)-1,3-dimethyl-1H-pyrazol-4-yl]ethylenesulfonamide

By a method similar to that in Reference Example 178, the title compound was obtained from 5-(5-chloro-1H-pyrrolo[2,3-b]pyridin-1-yl)-1,3-dimethyl-1H-pyrazole-4-carbaldehyde obtained in Reference Example 182 and tert-butyl {[(diphenylphosphoryl)methyl]sulfonyl}carbamate.

¹H-NMR (300 MHz, DMSO-d₆) δ:2.36 (s, 3H), 3.49 (s, 3H), 6.09 (d, J=15.9 Hz, 1H), 6.78 (d, J=15.9 Hz, 1H), 6.85-6.91 (m, 3H), 7.81 (d, J=3.4 Hz, 1H), 8.28-8.33 (m, 2H).

### Reference Example 184

### 2-[5-(5-chloro-1H-pyrrolo[2,3-b]pyridin-1-yl)-1,3-dimethyl-1H-pyrazol-4-yl]ethanesulfonamide

By a method similar to that in Reference Example 179, the title compound was obtained from (E)-2-[5-(5-chloro-1H-pyrrolo[2,3-b]pyridin-1-yl)-1,3-dimethyl-1H-pyrazol-4-yl]ethylenesulfonamide obtained in Reference Example 183.

¹H-NMR (300 MHz, DMSO-d₆) δ:2.21 (s, 3H), 2.53-2.60 (m, 2H), 2.86-2.94 (m, 2H), 3.41 (s, 3H), 6.74 (s, 2H), 6.80 (d, J=3.8 Hz, 1H), 7.80 (d, J=3.8 Hz, 1H), 8.27 (q, J=2.3 Hz, 2H).

### Reference Example 185

### (E)-2-[5-(5-fluoro-1H-indol-1-yl)-1,3-dimethyl-1H-pyrazol-4-yl]ethylenesulfonamide

By a method similar to that in Reference Example 178, the title compound was obtained from 5-(5-fluoro-1H-indol-1-yl)-1,3-dimethyl-1H-pyrazole-4-carbaldehyde obtained in Reference Example 20 and tert-butyl {[(diphenylphosphoryl)methyl]sulfonyl}carbamate.

¹H-NMR (300 MHz, CDCl₃) δ:2.43 (s, 3H), 3.54 (s, 3H), 4.49 (s, 2H), 5.87 (d, J=15.6 Hz, 1H), 6.78 (d, J=3.2 Hz, 1H), 6.88-6.94 (m, 1H), 6.96-7.04 (m, 1H), 7.10-7.18 (m, 2H), 7.36 (dd, J=9.1, 2.4 Hz, 1H).

### Reference Example 186

### 2-[5-(5-fluoro-1H-indol-1-yl)-1,3-dimethyl-1H-pyrazol-4-yl]ethanesulfonamide

By a method similar to that in Reference Example 179, the title compound was obtained from (E)-2-[5-(5-fluoro-1H-indol-1-yl)-1,3-dimethyl-1H-pyrazol-4-yl]ethylenesulfonamide obtained in Reference Example 185.

¹H-NMR (300 MHz, DMSO-d₆) δ:2.22 (s, 3H), 2.55-2.71 (m, 2H), 2.75-2.94 (m, 2H), 3.37 (s, 3H), 6.74-6.79 (m, 3H), 7.01-7.07 (m, 2H), 7.45-7.51 (m, 1H), 7.60 (d, J=3.4 Hz, 1H).

### Reference Example 187

### (E)-2-[1,3-dimethyl-5-(1H-pyrrolo[2,3-b]pyridin-1-yl)-1H-pyrazol-4-yl]ethylenesulfonamide

By a method similar to that in Reference Example 178, the title compound was obtained from 1,3-dimethyl-5-(1H-pyrrolo[2,3-b]pyridin-1-yl)-1H-pyrazole-4-carbaldehyde obtained in Reference Example 11 and tert-butyl {[(diphenylphosphoryl)methyl]sulfonyl}carbamate.

¹H-NMR (300 MHz, CDCl₃) δ:2.41 (s, 3H), 3.58 (s, 3H), 4.71 (s, 2 H), 6.07 (d, J=15.6 Hz, 1H), 6.78 (d, J=3.8 Hz, 1H), 7.12 (d, J=15.6 Hz, 1H), 7.17-7.23 (m, 2H), 8.03 (dd, J=7.9, 1.5 Hz, 1H), 8.33 (dd, J=4.7, 1.5 Hz, 1H).

### Reference Example 188

### 2-[1,3-dimethyl-5-(1H-pyrrolo[2,3-b]pyridin-1-yl)-1H-pyrazol-4-yl]ethanesulfonamide

By a method similar to that in Reference Example 179, the title compound was obtained from (E)-2-[1,3-dimethyl-5-(1H-pyrrolo[2,3-b]pyridin-1-yl)-1H-pyrazol-4-yl]ethylenesulfonamide obtained in Reference Example 187.

¹H-NMR (300 MHz, CDCl₃) δ:2.35 (s, 3H), 2.73-3.24 (m, 4H), 3.46 (s, 3H), 4.99 (s, 2H), 6.75 (d, J=3.6 Hz, 1H), 7.18 (d, J=3.8 Hz, 1H), 7.22 (dd, J=7.9, 4.9 Hz, 1H), 8.04 (dd, J=7.8, 1.6 Hz, 1H), 8.31 (dd, J=4.7, 1.5 Hz, 1H).

### Reference Example 189

### (E)-2-[5-(6-methoxy-1H-indol-1-yl)-1,3-dimethyl-1H-pyrazol-4-yl]ethylenesulfonamide

By a method similar to that in Reference Example 178, the title compound was obtained from 5-(6-methoxy-1H-indol-1-yl)-1,3-dimethyl-1H-pyrazole-4-carbaldehyde obtained in Reference Example 58 and tert-butyl {[(diphenylphosphoryl)methyl]sulfonyl}carbamate.

¹H-NMR (300 MHz, DMSO-d₆) δ:2.37 (s, 3H), 3.48 (s, 3H), 3.71 (s, 3H), 6.08 (d, J=15.9 Hz, 1H), 6.49 (d, J=2.3 Hz, 1H), 6.76 (d, J=3.4 Hz, 1H), 6.81-6.89 (m, 4H), 7.36 (d, J=3.4 Hz, 1H), 7.59 (d, J=8.7 Hz, 1H).

### Reference Example 190

### 2-[5-(6-methoxy-1H-indol-1-yl)-1,3-dimethyl-1H-pyrazol-4-yl]ethanesulfonamide

By a method similar to that in Reference Example 179, the title compound was obtained from (E)-2-[5-(6-methoxy-1H-indol-1-yl)-1,3-dimethyl-1H-pyrazol-4-yl]ethylenesulfonamide obtained in Reference Example 189.

¹H-NMR (300 MHz, CDCl₃) δ:2.30 (s, 3H), 2.71-2.84 (m, 2H), 2.86-2.97 (m, 2H), 3.49 (s, 3H), 3.77 (s, 3H), 4.52 (s, 2H), 6.43 (d, J=2.1 Hz, 1H), 6.64-6.67 (m, 1H), 6.85 (dd, J=8.7, 2.3 Hz, 1H), 6.95 (d, J=3.2 Hz, 1H), 7.54 (d, J=8.7 Hz, 1H).

### Reference Example 191

### 5-(5-chloro-1H-pyrrolo[2,3-b]pyridin-1-yl)-1-methyl-3-(trifluoromethyl)-1H-pyrazole-4-carbaldehyde

To a solution of 5-chloro-1H-pyrrolo[2,3-b]pyridine (1.22 g) in N,N-dimethylformamide (25 mL), which was cooled at 0° C. in an ice bath, was added 60% sodium hydride (in oil, 340 mg) with stirring, and the mixture was stirred at 0° C. for 20 min. 5-Chloro-1-methyl-3-(trifluoromethyl)-1H-pyrazole-4-carbaldehyde (1.51 g) was added to this reaction mixture at 0° C., and the reaction mixture was stirred at 100° C. for 3.5 hr. After the reaction mixture was allowed to cool to room temperature, water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 80:20, v/v) to give the title compound (1.71 g, yield 73%) as colorless crystals.

¹H-NMR (300 MHz, CDCl₃) δ:3.81 (s, 3H), 6.76 (d, J=3.6 Hz, 1H), 7.36 (d, J=3.6 Hz, 1H), 8.00 (d, J=2.3 Hz, 1H), 8.27 (d, J=2.3 Hz, 1H), 9.87 (s, 1H).

### Reference Example 192

### (E)-2-[5-(5-chloro-1H-pyrrolo[2,3-b]pyridin-1-yl)-1-methyl-3-(trifluoromethyl)-1H-pyrazol-4-yl]ethylenesulfonamide

To a solution of tert-butyl {[(diphenylphosphoryl)methyl]sulfonyl}carbamate (2.46 g) in N,N-dimethylformamide (50 mL) was added 60% sodium hydride (in oil, 645 mg) with stirring at 0° C., and the mixture was stirred at 0° C. for 1 hr. 5-(5-Chloro-1H-pyrrolo[2,3-b]pyridin-1-yl)-1-methyl-3-(trifluoromethyl)-1H-pyrazole-4-carbaldehyde obtained in Reference Example 191 (1.71 g) was added to the reaction mixture, and the mixture was stirred at room temperature for 1 hr. A saturated aqueous ammonium chloride solution (50 mL) was added to the reaction mixture, and the mixture was extracted with ethyl acetate. The organic layer was washed with saturated brine, dried over anhydrous magnesium sulfate, and filtrated. The filtrate was concentrated, trifluoroacetic acid (52 mL) was added to the residue, and the mixture was stirred at room temperature for 2 hr. The reaction mixture was concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 65:35, v/v), and crystallized from hexane-ethyl acetate to give the title compound (1.51 g, yield 72%) as colorless crystals.

¹H-NMR (300 MHz, DMSO-d₆) δ:3.66 (s, 3H), 5.86 (d, J=15.8 Hz, 1H), 6.89-7.08 (m, 4H), 7.91 (d, J=3.8 Hz, 1H), 8.32-8.38 (m, 2H).

### Reference Example 193

### 2-[5-(5-chloro-1H-pyrrolo[2,3-b]pyridin-1-yl)-1-methyl-3-(trifluoromethyl)-1H-pyrazol-4-yl]ethanesulfonamide

To a solution of (E)-2-[5-(5-chloro-1H-pyrrolo[2,3-b]pyridin-1-yl)-1-methyl-3-(trifluoromethyl)-1H-pyrazol-4-yl]ethylenesulfonamide obtained in Reference Example 192 (1.36 g) in a mixed solvent of tetrahydrofuran (30 mL) and ethanol (30 mL) was added 10% palladium carbon (136 mg), and the mixture was stirred under 1 atom of hydrogen atmosphere at room temperature for 8 hr. The catalyst was removed by filtration, and the filtrate was concentrated. To a solution of this residue in a mixed solvent of tetrahydrofuran (30 mL) and ethanol (30 mL) was added 10% palladium carbon (136 mg), and the mixture was stirred under 1 atom of hydrogen atmosphere at room temperature for 24 hr. The catalyst was removed by filtration, and the filtrate was concentrated. The residue was crystallized from hexane-ethyl acetate to give the title compound (1.22 g, yield 90%) as colorless crystals.

¹H-NMR (300 MHz, DMSO-d₆) δ:2.67-2.75 (m, 2H), 2.84-2.96 (m, 2H), 3.61 (s, 3H), 6.82-6.89 (m, 3H), 7.92 (d, J=3.8 Hz, 1H), 8.32 (s, 2H).

### Reference Example 194

### (E)-2-{1,3-dimethyl-5-[6-(trifluoromethyl)-1H-indol-1-yl]-1H-pyrazol-4-yl}ethylenesulfonamide

By a method similar to that in Reference Example 178, the title compound was obtained from 1,3-dimethyl-5-[6-(trifluoromethyl)-1H-indol-1-yl]-1H-pyrazole-4-carbaldehyde obtained in Reference Example 41 and tert-butyl {[(diphenylphosphoryl)methyl]sulfonyl}carbamate.

¹H-NMR (300 MHz, CDCl₃) δ:2.46 (s, 3H), 3.55 (s, 3H), 4.43 (s, 2H), 5.88 (d, J=15.6 Hz, 1H), 6.90 (d, J=3.4 Hz, 1H), 7.14 (d, J=15.6 Hz, 1H), 7.23-7.27 (m, 2H), 7.50 (d, J=8.5 Hz, 1H), 7.83 (d, J=8.3 Hz, 1H).

### Reference Example 195

### 2-{1,3-dimethyl-5-[6-(trifluoromethyl)-1H-indol-1-yl]-1H-pyrazol-4-yl}ethanesulfonamide

By a method similar to that in Reference Example 179, the title compound was obtained from (E)-2-{1,3-dimethyl-5-[6-(trifluoromethyl)-1H-indol-1-yl]-1H-pyrazol-4-yl}ethylenesulfonamide obtained in Reference Example 194.

¹H-NMR (300 MHz, DMSO-d₆) δ:2.24 (s, 3H), 2.42-2.54 (m, 1H), 2.60-2.73 (m, 1H), 2.77-2.95 (m, 2H), 3.38 (s, 3H), 6.76 (s, 2H), 6.94 (d, J=3.0 Hz, 1H), 7.32 (s, 1H), 7.49 (d, J=8.3 Hz, 1H), 7.80 (d, J=3.4 Hz, 1H), 7.93 (d, J=8.3 Hz, 1H).

### Reference Example 196

### butyl sulfamate

Formic acid (930 mg) was added to chlorosulfonyl isocyanate (2.86 g) cooled at 0° C. in an ice bath, and the mixture was vigorously stirred for 5 min. Acetonitrile (10 mL) was added to the reaction mixture, and the mixture was stirred at 0° C. for 1 hr, and then at room temperature for 7 hr. A solution of butanol (1.00 g) and pyridine (1.60 g) in acetonitrile (9 mL) was added to the reaction mixture, and the mixture was stirred at room temperature for 24 hr. Water was added to the reaction mixture, and the mixture was extracted with ethyl acetate. The organic layer was washed with saturated brine, dried over anhydrous magnesium sulfate, and filtrated. The filtrate was concentrated to give the title compound (2.09 g, yield 99%) as a colorless oil.

¹H-NMR (300 MHz, CDCl₃) δ:0.91-0.99 (m, 3H), 1.38-1.52 (m, 2H), 1.67-1.79 (m, 2H), 4.22 (t, J=6.5 Hz, 2H), 4.98 (br s, 2H).

### Reference Example 197

### N-{[5-(5-chloro-1H-indol-1-yl)-1,3-dimethyl-1H-pyrazol-4-yl]methyl}sulfamide

To a solution of 5-(5-chloro-1H-indol-1-yl)-1,3-dimethyl-1H-pyrazole-4-carbaldehyde obtained in Reference Example 37 (321 mg) in ethanol (5.8 mL) was added sulfamide (946 mg), and the mixture was heated under reflux for 24 hr. Sodium borohydride (48.8 mg) was added to the reaction mixture, and the mixture was stirred at room temperature for 2 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 35:65, v/v), and crystallized from hexane-ethyl acetate to give the title compound (100 mg, yield 24%) as colorless crystals.

¹H-NMR (300 MHz, CDCl₃) δ:2.36 (s, 3H), 3.51 (s, 3H), 3.85-4.00 (m, 2H), 4.04-4.15 (m, 3H), 6.71 (d, J=3.4 Hz, 1H), 6.95 (d, J=8.7 Hz, 1H), 7.15 (d, J=3.2 Hz, 1H), 7.22 (dd, J=8.8, 2.0 Hz, 1H), 7.67 (d, J=1.7 Hz, 1H).

### Reference Example 198

### 5-(5-chloro-6-methoxy-1H-indol-1-yl)-1,3-dimethyl-1H-pyrazole-4-carbaldehyde

By a method similar to that in Reference Example 1, the title compound was obtained from 5-chloro-6-methoxy-1H-indole and 5-chloro-1,3-dimethyl-1H-pyrazole-4-carbaldehyde.

¹H-NMR (300 MHz, CDCl₃) δ:2.58 (s, 3H), 3.59 (s, 3H), 3.85 (s, 3H), 6.55 (s, 1H), 6.70 (d, J=3.2 Hz, 1H), 7.09 (d, J=3.2 Hz, 1H), 7.70 (s, 1H), 9.55 (s, 1H).

### Reference Example 199

### (E)-2-[5-(5-chloro-6-methoxy-1H-indol-1-yl)-1,3-dimethyl-1H-pyrazol-4-yl]ethylenesulfonamide

By a method similar to that in Reference Example 178, the title compound was obtained from 5-(5-chloro-6-methoxy-1H-indol-1-yl)-1,3-dimethyl-1H-pyrazole-4-carbaldehyde obtained in Reference Example 198 and tert-butyl {[(diphenylphosphoryl)methyl]sulfonyl}carbamate.

¹H-NMR (300 MHz, CDCl₃) δ:2.45 (s, 3H), 3.55 (s, 3H), 3.83 (s, 3H), 4.49 (s, 2H), 5.92 (d, J=15.6 Hz, 1H), 6.44 (s, 1H), 6.70 (dd, J=3.4, 0.8 Hz, 1H), 6.98 (d, J=3.2 Hz, 1H), 7.17 (d, J=15.6 Hz, 1H), 7.70 (s, 1H).

### Reference Example 200

### 2-[5-(5-chloro-6-methoxy-1H-indol-1-yl)-1,3-dimethyl-1H-pyrazol-4-yl]ethanesulfonamide

By a method similar to that in Reference Example 179, the title compound was obtained from (E)-2-[5-(5-chloro-6-methoxy-1H-indol-1-yl)-1,3-dimethyl-1H-pyrazol-4-yl]ethylenesulfonamide obtained in Reference Example 199.

¹H-NMR (300 MHz, DMSO-d₆) δ:2.23 (s, 3H), 2.54-2.70 (m, 2H), 2.78-2.93 (m, 2H), 3.39 (s, 3H), 3.80 (s, 3H), 6.64 (s, 1H), 6.68 (d, J=3.4 Hz, 1H), 6.77 (s, 2H), 7.41 (d, J=3.4 Hz, 1H), 7.75 (s, 1H).

### Reference Example 201

### 1,3-dimethyl-5-[5-(trifluoromethyl)-1H-pyrrolo[2,3-b]pyridin-1-yl]-1H-pyrazole-4-carbaldehyde

To a solution of 5-(trifluoromethyl)-1H-pyrrolo[2,3-b]pyridine (8.08 g) in N,N-dimethylformamide (80 mL), which was cooled at 0° C. in an ice bath, was added 60% sodium hydride (in oil, 1.89 g) with stirring, and the mixture was stirred at 0° C. for 30 min. 5-Chloro-1,3-dimethyl-1H-pyrazole-4-carbaldehyde (6.25 g) was added to this reaction mixture at 0° C., and the reaction mixture was stirred at 80° C. for 8 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30, v/v), and crystallized from hexane-ethyl acetate to give the title compound (8.40 g, yield 69%) as colorless crystals.

¹H-NMR (300 MHz, CDCl₃) δ:2.57 (s, 3H), 3.68 (s, 3H), 6.89 (d, J=3.8 Hz, 1H), 7.46 (d, J=3.6 Hz, 1H), 8.30 (d, J=1.5 Hz, 1H), 8.62 (d, J=1.3 Hz, 1H), 9.63 (s, 1H).

### Reference Example 202

### ethyl (2E)-3-{1,3-dimethyl-5-[5-(trifluoromethyl)-1H-pyrrolo[2,3-b]pyridin-1-yl]-1H-pyrazol-4-yl}acrylate

By a method similar to that in Reference Example 12, the title compound was obtained from 1,3-dimethyl-5-[5-(trifluoromethyl)-1H-pyrrolo[2,3-b]pyridin-1-yl]-1H-pyrazole-4-carbaldehyde obtained in Reference Example 201 and ethyl (diethoxyphosphoryl)acetate.

¹H-NMR (300 MHz, CDCl₃) δ:1.23 (t, J=7.2 Hz, 3H), 2.47 (s, 3H), 3.58 (s, 3H), 4.14 (q, J=7.2 Hz, 2H), 5.71 (d, J=16.3 Hz, 1H), 6.88 (d, J=3.8 Hz, 1H), 7.23-7.30 (m, 1H), 7.35 (d, J=3.8 Hz, 1H), 8.31 (d, J=1.9 Hz, 1H), 8.62 (d, J=1.9 Hz, 1H).

### Reference Example 203

### (2E)-3-{1,3-dimethyl-5-[5-(trifluoromethyl)-1H-pyrrolo[2,3-b]pyridin-1-yl]-1H-pyrazol-4-yl}acrylic acid

By a method similar to that in Reference Example 13, the title compound was obtained from ethyl (2E)-3-{1,3-dimethyl-5-[5-(trifluoromethyl)-1H-pyrrolo[2,3-b]pyridin-1-yl]-1H-pyrazol-4-yl}acrylate obtained in Reference Example 202.

¹H-NMR (300 MHz, DMSO-d₆) δ:2.39 (s, 3H), 3.51 (s, 3H), 5.48 (d, J=16.3 Hz, 1H), 7.04 (d, J=3.4 Hz, 1H), 7.05 (d, J=16.3 Hz, 1H), 7.97 (d, J=3.4 Hz, 1H), 8.65 (d, J=4.5 Hz, 2H), 12.21 (br s, 1H).

### Reference Example 204

### 1,3-dimethyl-5-(5-methyl-1H-pyrrolo[2,3-b]pyridin-1-yl)-1H-pyrazole-4-carbaldehyde

To a solution of 5-methyl-1H-pyrrolo[2,3-b]pyridine (1.70 g) in N,N-dimethylformamide (30 mL), which was cooled at 0° C. in an ice bath, was added 60% sodium hydride (in oil, 561 mg) with stirring, and the mixture was stirred at 0° C. for 30 min. 5-Chloro-1,3-dimethyl-1H-pyrazole-4-carbaldehyde (1.85 g) was added to this reaction mixture at 0° C., and the reaction mixture was stirred at 80° C. for 6 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30, v/v), and crystallized from hexane-ethyl acetate to give the title compound (1.42 g, yield 48%) as colorless crystals.

¹H-NMR (300 MHz, CDCl₃) δ:2.46 (s, 3H), 2.54 (s, 3H), 3.68 (s, 3H), 6.69 (d, J=3.6 Hz, 1H), 7.25-7.29 (m, 1H), 7.80-7.83 (m, 1H), 8.19 (d, J=2.1 Hz, 1H), 9.57 (s, 1H).

### Reference Example 205

### ethyl (2E)-3-[1,3-dimethyl-5-(5-methyl-1H-pyrrolo[2,3-b]pyridin-1-yl)-1H-pyrazol-4-yl]acrylate

To a solution of ethyl (diethoxyphosphoryl)acetate (1.38 g) in tetrahydrofuran (46 mL), which was cooled at 0° C. in an ice bath, was added 60% sodium hydride (in oil, 269 mg) with stirring, and the mixture was stirred at 0° C. for 30 min. A solution of 1,3-dimethyl-5-(5-methyl-1H-pyrrolo[2,3-b]pyridin-1-yl)-1H-pyrazole-4-carbaldehyde obtained in Reference Example 204 (1.42 g) in tetrahydrofuran (10 mL) was added to this reaction mixture at 0° C., and the reaction mixture was stirred at 0° C. for 4 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v) to give the title compound (1.61 g, yield 89%) as pale-yellow crystals.

¹H-NMR (300 MHz, CDCl₃) δ:1.23 (t, J=7.2 Hz, 3H), 2.44-2.47 (m, 6H), 3.58 (s, 3H), 4.13 (q, J=7.1 Hz, 2H), 5.69 (d, J=16.3 Hz, 1H), 6.68 (d, J=3.4 Hz, 1H), 7.15 (d, J=3.8 Hz, 1H), 7.30 (d, J=16.3 Hz, 1H), 7.81 (d, J=1.5 Hz, 1H), 8.18 (d, J=1.5 Hz, 1H).

### Reference Example 206

### (2E)-3-[1,3-dimethyl-5-(5-methyl-1H-pyrrolo[2,3-b]pyridin-1-yl)-1H-pyrazol-4-yl]acrylic acid

To a solution of ethyl (2E)-3-[1,3-dimethyl-5-(5-methyl-1H-pyrrolo[2,3-b]pyridin-1-yl)-1H-pyrazol-4-yl]acrylate obtained in Reference Example 205 (1.59 g) in a mixed solvent of tetrahydrofuran (25 mL) and ethanol (25 mL) was added a 1N aqueous sodium hydroxide solution (9.8 mL), and the mixture was stirred with heating at 60° C. for 3 hr. The reaction mixture was allowed to cool to room temperature, and concentrated. An aqueous solution (20 mL) of the residue was neutralized with an aqueous solution (15 mL) of potassium hydrogensulfate (1.33 g), and the precipitated crystals were collected by filtration. The crystals were dissolved in ethyl acetate, and the solution was dried over anhydrous magnesium sulfate, and filtrated. The filtrate was concentrated, and the residue was crystallized from hexane-ethanol to give the title compound (1.14 g, yield 79%) as colorless crystals.

¹H-NMR (300 MHz, DMSO-d₆) δ:2.37 (s, 3H), 2.41 (s, 3H), 3.48 (s, 3H), 5.47 (d, J=16.3 Hz, 1H), 6.79 (d, J=3.8 Hz, 1H), 7.06 (d, J=16.3 Hz, 1H), 7.65 (d, J=3.4 Hz, 1H), 7.95 (s, 1H), 8.12 (d, J=1.9 Hz, 1H), 12.14 (br s, 1H).

### Reference Example 207

### (E)-2-{1,3-dimethyl-5-[5-(trifluoromethyl)-1H-pyrrolo[2,3-b]pyridin-1-yl]-1H-pyrazol-4-yl}ethylenesulfonamide

A solution of tert-butyl {[(diphenylphosphoryl)methyl]sulfonyl}carbamate (8.92 g) in N,N-dimethylformamide (180 mL) was added 60% sodium hydride (in oil, 2.33 g) with stirring at 0° C., and the mixture was stirred at 0° C. for 1 hr. 1,3-Dimethyl-5-[5-(trifluoromethyl)-1H-pyrrolo[2,3-b]pyridin-1-yl]-1H-pyrazole-4-carbaldehyde obtained in Reference Example 201 (5.79 g) was added to the reaction mixture, and the mixture was stirred at room temperature for 1 hr. A saturated aqueous ammonium chloride solution (100 mL) was added to the reaction mixture, and the mixture was extracted with ethyl acetate. The organic layer was washed with saturated brine, dried over anhydrous magnesium sulfate, and filtrated. The filtrate was concentrated, trifluoroacetic acid (50 mL) was added to the residue, and the mixture was stirred at room temperature for 2 hr. The reaction mixture was concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v), and crystallized from hexane-ethyl acetate to give the title compound (6.36 g, yield 88%) as colorless crystals.

¹H-NMR (300 MHz, DMSO-d₆) δ:2.38 (s, 3H), 3.50 (s, 3H), 6.12 (d, J=15.9 Hz, 1H), 6.78 (d, J=15.9 Hz, 1H), 6.86 (s, 2H), 7.06 (d, J=3.8 Hz, 1H), 7.95 (d, J=3.8 Hz, 1H), 8.60 (s, 1H), 8.65 (s, 1H).

### Reference Example 208

### 2-{1,3-dimethyl-5-[5-(trifluoromethyl)-1H-pyrrolo[2,3-b]pyridin-1-yl]-1H-pyrazol-4-yl}ethanesulfonamide

To a solution of (E)-2-{1,3-dimethyl-5-[5-(trifluoromethyl)-1H-pyrrolo[2,3-b]pyridin-1-yl]-1H-pyrazol-4-yl}ethylenesulfonamide obtained in Reference Example 207 (6.31 g) in a mixed solvent of tetrahydrofuran (80 mL) and ethanol (80 mL) was added 10% palladium carbon (631 mg), and the mixture was stirred under 1 atom of hydrogen atmosphere at room temperature for 8 hr. The catalyst was removed by filtration, and the filtrate was concentrated. To a solution of this residue in a mixed solvent of tetrahydrofuran (80 mL) and ethanol (80 mL) was added 10% palladium carbon (631 mg), and the mixture was stirred under 1 atom of hydrogen atmosphere at room temperature for 5 hr. The catalyst was removed by filtration, and the filtrate was concentrated. The residue was crystallized from hexane-ethanol to give the title compound (6.11 g, yield 96%) as colorless crystals.

¹H-NMR (300 MHz, DMSO-d₆) δ:2.22 (s, 3H), 2.53-2.63 (m, 2H), 2.92 (t, J=8.3 Hz, 2H), 3.43 (s, 3H), 6.74 (s, 2H), 6.98 (d, J=3.4 Hz, 1H), 7.93 (d, J=3.4 Hz, 1H), 8.60 (s, 1H), 8.65 (s, 1H).

### Reference Example 209

### (E)-2-[1,3-dimethyl-5-(5-methyl-1H-pyrrolo[2,3-b]pyridin-1-yl)-1H-pyrazol-4-yl]ethylenesulfonamide

By a method similar to that in Reference Example 178, the title compound was obtained from 1,3-dimethyl-5-(5-methyl-1H-pyrrolo[2,3-b]pyridin-1-yl)-1H-pyrazole-4-carbaldehyde obtained in Reference Example 204 and tert-butyl {[(diphenylphosphoryl)methyl]sulfonyl}carbamate.

¹H-NMR (300 MHz, DMSO-d₆) δ:2.36 (s, 3H), 2.41 (s, 3H), 3.47 (s, 3H), 6.12 (d, J=15.6 Hz, 1H), 6.76-6.89 (m, 4H), 7.63 (d, J=3.6 Hz, 1H), 7.95 (d, J=1.1 Hz, 1H), 8.13 (d, J=1.5 Hz, 1H).

### Reference Example 210

### 2-[1,3-dimethyl-5-(5-methyl-1H-pyrrolo[2,3-b]pyridin-1-yl)-1H-pyrazol-4-yl]ethanesulfonamide

By a method similar to that in Reference Example 179, the title compound was obtained from (E)-2-[1,3-dimethyl-5-(5-methyl-1H-pyrrolo[2,3-b]pyridin-1-yl)-1H-pyrazol-4-yl]ethylenesulfonamide obtained in Reference Example 209.

¹H-NMR (300 MHz, DMSO-d₆) δ:2.20 (s, 3H), 2.40 (s, 3H), 2.53-2.62 (m, 2H), 2.85-2.93 (m, 2H), 3.39 (s, 3H), 6.71 (d, J=3.6 Hz, 1H), 6.75 (s, 2H), 7.63 (d, J=3.8 Hz, 1H), 7.89-7.93 (m, 1H), 8.11 (d, J=2.1 Hz, 1H).

### Reference Example 211

### ethyl 3-{1,3-dimethyl-5-[5-(trifluoromethyl)-1H-pyrrolo[2,3-b]pyridin-1-yl]-1H-pyrazol-4-yl}propanoate

By a method similar to that in Reference Example 65, the title compound was obtained from ethyl (2E)-3-{1,3-dimethyl-5-[5-(trifluoromethyl)-1H-pyrrolo[2,3-b]pyridin-1-yl]-1H-pyrazol-4-yl}acrylate obtained in Reference Example 202.

¹H-NMR (300 MHz, CDCl₃) δ:1.16 (t, J=7.1 Hz, 3H), 2.26-2.35 (m, 5H), 2.54-2.63 (m, 2H), 3.51 (s, 3H), 3.99 (q, J=7.0 Hz, 2H), 6.82 (d, J=3.6 Hz, 1H), 7.37 (d, J=3.6 Hz, 1H), 8.27 (d, J=1.5 Hz, 1H), 8.60 (d, J=1.9 Hz, 1H).

### Reference Example 212

### 3-{1,3-dimethyl-5-[5-(trifluoromethyl)-1H-pyrrolo[2,3-b]pyridin-1-yl]-1H-pyrazol-4-yl}propan-1-ol

By a method similar to that in Reference Example 66, the title compound was obtained from ethyl 3-{1,3-dimethyl-5-[5-(trifluoromethyl)-1H-pyrrolo[2,3-b]pyridin-1-yl]-1H-pyrazol-4-yl}propanoate obtained in Reference Example 211.

¹H-NMR (300 MHz, CDCl₃) δ:1.53-1.65 (m, 2H), 2.29-2.40 (m, 5H), 3.43-3.52 (m, 5H), 6.81 (d, J=3.6 Hz, 1H), 7.34 (d, J=3.8 Hz, 1H), 8.27 (d, J=1.7 Hz, 1H), 8.60 (d, J=1.7 Hz, 1H).

### Reference Example 213

### 5-(2,3-dihydro-1H-pyrrolo[2,3-b]pyridin-1-yl)-1,3-dimethyl-1H-pyrazole-4-carbaldehyde

1,3-Dimethyl-5-(1H-pyrrolo[2,3-b]pyridin-1-yl)-1H-pyrazole-4-carbaldehyde obtained in Reference Example 11 (1.38 g) was dissolved in methanol (100 mL), 10% palladium carbon (530 mg) was added, and the mixture was stirred under 1 atom of hydrogen atmosphere at room temperature for 78 hr. The catalyst was removed by filtration, and the filtrate was concentrated. The residue was subjected to silica gel column chromatography (ethyl acetate) to give the title compound (0.85 g, yield 61%) as colorless crystals.

¹H-NMR (300 MHz, CDCl₃) δ:2.47 (s, 3H), 3.28 (t, J=8.6 Hz, 2H), 3.73 (s, 3H), 4.04 (t, J=8.6 Hz, 2H), 6.67 (dd, J=6.9, 5.5 Hz, 1H), 7.40 (d, J=6.9 Hz, 1H), 7.89 (d, J=5.5 Hz, 1H), 9.78 (s, 1H).

### Reference Example 214

### ethyl (2E)-3-[5-(2,3-dihydro-1H-pyrrolo[2,3-b]pyridin-1-yl)-1,3-dimethyl-1H-pyrazol-4-yl]acrylate

By a method similar to that in Reference Example 12, the title compound was obtained from 5-(2,3-dihydro-1H-pyrrolo[2,3-b]pyridin-1-yl)-1,3-dimethyl-1H-pyrazole-4-carbaldehyde obtained in Reference Example 213 and ethyl (diethoxyphosphoryl)acetate.

¹H-NMR (300 MHz, CDCl₃) δ:1.23-1.32 (m, 3H), 2.38 (s, 3H), 3.23-3.35 (m, 2H), 3.66 (s, 3H), 3.75-3.95 (m, 2H), 4.15-4.21 (m, 2H), 5.96 (d, J=16.2 Hz, 1H), 6.65 (dd, J=7.2, 5.3 Hz, 1H), 7.39 (dd, J=7.2, 1.5 Hz, 1H), 7.45 (d, J=16.2 Hz, 1H), 7.90 (dd, J=5.3, 1.5 Hz, 1H).

### Reference Example 215

### (2E)-3-[5-(2,3-dihydro-1H-pyrrolo[2,3-b]pyridin-1-yl)-1,3-dimethyl-1H-pyrazol-4-yl]acrylic acid

By a method similar to that in Reference Example 13, the title compound was obtained from ethyl (2E)-3-[5-(2,3-dihydro-1H-pyrrolo[2,3-b]pyridin-1-yl)-1,3-dimethyl-1H-pyrazol-4-yl]acrylate obtained in Reference Example 214.

¹H-NMR (300 MHz, DMSO-d₆) δ:2.27 (s, 3H), 3.26 (t, J=8.5 Hz, 2H), 3.56 (s, 3H), 3.79-3.95 (m, 2H), 5.80 (d, J=16.1 Hz, 1H), 6.68 (dd, J=7.2, 4.5 Hz, 1H), 7.24 (d, J=16.1 Hz, 1H), 7.50 (dd, J=7.2, 1.5 Hz, 1H), 7.76 (d, J=4.5 Hz, 1H), 12.08 (s, 1H).

### Reference Example 216

### 3-cyclopropyl-1-methyl-5-(1H-pyrrolo[2,3-b]pyridin-1-yl)-1H-pyrazole-4-carbaldehyde

By a method similar to that in Reference Example 1, the title compound was obtained from 1H-pyrrolo[2,3-b]pyridine and 5-chloro-3-cyclopropyl-1-methyl-1H-pyrazole-4-carbaldehyde obtained in Reference Example 150.

¹H-NMR (300 MHz, CDCl₃) δ:0.99-1.08 (m, 4H), 2.45-2.55 (m, 1H), 3.63 (s, 3H), 6.76 (d, J=3.8 Hz, 1H), 7.22 (dd, J=7.8, 4.8 Hz, 1H), 7.31 (d, J=3.8 Hz, 1H), 8.02 (dd, J=7.8, 1.5 Hz, 1H), 8.36 (dd, J=4.8, 1.5 Hz, 1H), 9.66 (s, 1H).

### Reference Example 217

### ethyl (2E)-3-[3-cyclopropyl-1-methyl-5-(1H-pyrrolo[2,3-b]pyridin-1-yl)-1H-pyrazol-4-yl]acrylate

By a method similar to that in Reference Example 12, the title compound was obtained from 3-cyclopropyl-1-methyl-5-(1H-pyrrolo[2,3-b]pyridin-1-yl)-1H-pyrazole-4-carbaldehyde obtained in Reference Example 216 and ethyl (diethoxyphosphoryl)acetate.

¹H-NMR (300 MHz, CDCl₃) δ:1.00 (d, J=7.2 Hz, 4H), 1.23 (t, J=7.1 Hz, 3H), 1.91-2.05 (m, 1H), 3.55 (s, 3H), 4.13 (q, J=7.1 Hz, 2H), 5.92 (d, J=16.1 Hz, 1H), 6.76 (d, J=3.8 Hz, 1H), 7.15-7.24 (m, 2H), 7.40 (d, J=16.1 Hz, 1H), 8.02 (dd, J=7.8, 1.7 Hz, 1H), 8.35 (dd, J=4.5, 1.7 Hz, 1H).

### Reference Example 218

### (2E)-3-[3-cyclopropyl-1-methyl-5-(1H-pyrrolo[2,3-b]pyridin-1-yl)-1H-pyrazol-4-yl]acrylic acid

By a method similar to that in Reference Example 13, the title compound was obtained from ethyl (2E)-3-[3-cyclopropyl-1-methyl-5-(1H-pyrrolo[2,3-b]pyridin-1-yl)-1H-pyrazol-4-yl]acrylate obtained in Reference Example 217.

¹H-NMR (300 MHz, DMSO-d₆) δ:0.85-0.99 (m, 4H), 2.00-2.10 (m, 1H), 3.47 (s, 3H), 5.59 (d, J=16.2 Hz, 1H), 6.88 (d, J=3.6 Hz, 1H), 7.18 (d, J=16.2 Hz, 1H), 7.28 (dd, J=7.9, 4.7 Hz, 1H), 7.72 (d, J=3.6 Hz, 1H), 8.17 (dd, J=7.9, 1.6 Hz, 1H), 8.28 (dd, J=4.7, 1.6 Hz, 1H), 12.14 (s, 1H).

### Reference Example 219

### 3-cyclopropyl-5-(2,3-dihydro-1H-pyrrolo[2,3-b]pyridin-1-yl)-1-methyl-1H-pyrazole-4-carbaldehyde

By a method similar to that in Reference Example 213, the title compound was obtained from 3-cyclopropyl-1-methyl-5-(1H-pyrrolo[2,3-b]pyridin-1-yl)-1H-pyrazole-4-carbaldehyde obtained in Reference Example 216.

¹H-NMR (300 MHz, CDCl₃) δ:0.93-1.05 (m, 4H), 2.03-2.31 (m, 1H), 3.27 (t, J=8.5 Hz, 2H), 3.68 (s, 3H), 3.94-4.10 (m, 2 H), 6.66 (dd, J=7.2, 5.1 Hz, 1H), 7.35-7.42 (m, 1H), 7.88 (d, J=5.1 Hz, 1H), 9.91 (s, 1H).

### Reference Example 220

### ethyl (2E)-3-[3-cyclopropyl-5-(2,3-dihydro-1H-pyrrolo[2,3-b]pyridin-1-yl)-1-methyl-1H-pyrazol-4-yl]acrylate

By a method similar to that in Reference Example 12, the title compound was obtained from 3-cyclopropyl-5-(2,3-dihydro-1H-pyrrolo[2,3-b]pyridin-1-yl)-1-methyl-1H-pyrazole-4-carbaldehyde obtained in Reference Example 219 and ethyl (diethoxyphosphoryl)acetate.

¹H-NMR (300 MHz, CDCl₃) δ:0.92-0.96 (m, 4H), 1.27 (t, J=7.1 Hz, 3H), 1.87-1.96 (m, 1H), 3.23-3.32 (m, 2H), 3.62 (s, 3H), 3.78-3.95 (m, 2H), 4.18 (q, J=7.1 Hz, 2H), 6.18 (d, J=16.1 Hz, 1H), 6.64 (dd, J=7.2, 5.3 Hz, 1H), 7.38 (dd, J=7.2, 1.5 Hz, 1H), 7.54 (d, J=16.1 Hz, 1H), 7.90 (d, J=5.3 Hz, 1H).

### Reference Example 221

### (2E)-3-[3-cyclopropyl-5-(2,3-dihydro-1H-pyrrolo[2,3-b]pyridin-1-yl)-1-methyl-1H-pyrazol-4-yl]acrylic acid

By a method similar to that in Reference Example 13, the title compound was obtained from ethyl (2E)-3-[3-cyclopropyl-5-(2,3-dihydro-1H-pyrrolo[2,3-b]pyridin-1-yl)-1-methyl-1H-pyrazol-4-yl]acrylate obtained in Reference Example 220.

¹H-NMR (300 MHz, DMSO-d₆) δ:0.75-0.83 (m, 2H), 0.85-0.94 (m, 2H), 1.90-1.99 (m, 1H), 3.25 (t, J=8.3 Hz, 2H), 3.54 (s, 3H), 3.78-3.93 (m, 2H), 5.98 (d, J=16.3 Hz, 1H), 6.68 (dd, J=7.2, 5.3 Hz, 1H), 7.34 (d, J=16.3 Hz, 1H), 7.45-7.53 (m, 1H), 7.74-7.77 (m, 1H), 12.08 (s, 1H).

### Reference Example 222

### 1,3-dimethyl-5-(1H-pyrrolo[3,2-c]pyridin-1-yl)-1H-pyrazole-4-carbaldehyde

By a method similar to that in Reference Example 1, the title compound was obtained from 1H-pyrrolo[3,2-c]pyridine and 5-chloro-1,3-dimethyl-1H-pyrazole-4-carbaldehyde.

¹H-NMR (300 MHz, CDCl₃) δ:2.57 (s, 3H), 3.59 (s, 3H), 6.93 (dd, J=3.4, 0.8 Hz, 1H), 7.05 (d, J=5.7 Hz, 1H), 7.24 (d, J=3.4 Hz, 1H), 8.44 (d, J=5.7 Hz, 1H), 9.06 (d, J=0.8 Hz, 1H), 9.56 (s, 1H).

### Reference Example 223

### ethyl (2E)-3-[1,3-dimethyl-5-(1H-pyrrolo[3,2-c]pyridin-1-yl)-1H-pyrazol-4-yl]acrylate

By a method similar to that in Reference Example 12, the title compound was obtained from 1,3-dimethyl-5-(1H-pyrrolo[3,2-c]pyridin-1-yl)-1H-pyrazole-4-carbaldehyde obtained in Reference Example 222 and ethyl (diethoxyphosphoryl)acetate.

¹H-NMR (300 MHz, CDCl₃) δ:1.21 (t, J=7.2 Hz, 3H), 2.47 (s, 3H), 3.51 (s, 3H), 4.12 (q, J=7.2 Hz, 2H), 5.60 (d, J=16.2 Hz, 1H), 6.92 (dd, J=3.4, 0.9 Hz, 1H), 6.97 (d, J=5.8 Hz, 1H), 7.14 (d, J=3.4 Hz, 1H), 7.26 (d, J=16.2 Hz, 1H), 8.40 (d, J=5.8 Hz, 1H), 9.05 (d, J=0.9 Hz, 1H).

### Reference Example 224

### (2E)-3-[1,3-dimethyl-5-(1H-pyrrolo[3,2-c]pyridin-1-yl)-1H-pyrazol-4-yl]acrylic acid

By a method similar to that in Reference Example 13, the title compound was obtained from ethyl (2E)-3-[1,3-dimethyl-5-(1H-pyrrolo[3,2-c]pyridin-1-yl)-1H-pyrazol-4-yl]acrylate obtained in Reference Example 223.

¹H-NMR (300 MHz, DMSO-d₆) δ:2.38 (s, 3H), 3.50 (s, 3H), 5.37 (d, J=16.2 Hz, 1H), 7.02-7.08 (m, 2H), 7.11 (d, J=5.8 Hz, 1H), 7.71 (d, J=3.4 Hz, 1H), 8.30 (d, J=5.8 Hz, 1H), 9.01 (s, 1H), 12.19 (s, 1H).

### Reference Example 225

### 1,3-dimethyl-5-(1H-pyrrolo[2,3-c]pyridin-1-yl)-1H-pyrazole-4-carbaldehyde

By a method similar to that in Reference Example 1, the title compound was obtained from 1H-pyrrolo[2,3-c]pyridine and 5-chloro-1,3-dimethyl-1H-pyrazole-4-carbaldehyde.

¹H-NMR (300 MHz, CDCl₃) δ:2.58 (s, 3H), 3.62 (s, 3H), 6.84 (d, J=3.0 Hz, 1H), 7.34 (d, J=3.0 Hz, 1H), 7.64 (d, J=5.5 Hz, 1H), 8.43 (d, J=5.5 Hz, 1H), 8.53 (s, 1H), 9.59 (s, 1H).

### Reference Example 226

### ethyl (2E)-3-[1,3-dimethyl-5-(1H-pyrrolo[2,3-c]pyridin-1-yl)-1H-pyrazol-4-yl]acrylate

By a method similar to that in Reference Example 12, the title compound was obtained from 1,3-dimethyl-5-(1H-pyrrolo[2,3-c]pyridin-1-yl)-1H-pyrazole-4-carbaldehyde obtained in Reference Example 225 and ethyl (diethoxyphosphoryl)acetate.

¹H-NMR (300 MHz, CDCl₃) δ:1.22 (t, J=7.2 Hz, 3H), 2.48 (s, 3H), 3.54 (s, 3H), 4.12 (q, J=7.2 Hz, 2H), 5.63 (d, J=16.4 Hz, 1H), 6.84 (dd, J=3.3, 0.9 Hz, 1H), 7.24 (s, 1H), 7.27 (d, J=16.4 Hz, 1H), 7.64 (dd, J=5.5, 0.9 Hz, 1H), 8.41 (d, J=5.5 Hz, 1H), 8.45 (s, 1H).

### Reference Example 227

### (2E)-3-[1,3-dimethyl-5-(1H-pyrrolo[2,3-c]pyridin-1-yl)-1H-pyrazol-4-yl]acrylic acid

By a method similar to that in Reference Example 13, the title compound was obtained from ethyl (2E)-3-[1,3-dimethyl-5-(1H-pyrrolo[2,3-c]pyridin-1-yl)-1H-pyrazol-4-yl]acrylate obtained in Reference Example 226.

¹H-NMR (300 MHz, DMSO-d₆) δ:2.39 (s, 3H), 3.53 (s, 3H), 5.36 (d, J=16.2 Hz, 1H), 6.95 (dd, J=3.3, 0.8 Hz, 1H), 7.06 (d, J=16.2 Hz, 1H), 7.74 (dd, J=5.4, 0.8 Hz, 1H), 7.85 (d, J=3.3 Hz, 1H), 8.31 (d, J=5.4 Hz, 1H), 8.41 (s, 1H), 12.19 (s, 1H).

### Reference Example 228

### 1-methyl-5-(1H-pyrrolo[2,3-b]pyridin-1-yl)-3-(trifluoromethyl)-1H-pyrazole-4-carbaldehyde

By a method similar to that in Reference Example 1, the title compound was obtained from 1H-pyrrolo[2,3-b]pyridine and 5-chloro-1-methyl-3-(trifluoromethyl)-1H-pyrazole-4-carbaldehyde.

¹H-NMR (300 MHz, CDCl₃) δ:3.82 (s, 3H), 6.80 (d, J=3.9 Hz, 1H), 7.22-7.26 (m, 1H), 7.32 (d, J=3.9 Hz, 1H), 8.01-8.05 (m, 1H), 8.32-8.35 (m, 1H), 9.83 (s, 1H).

### Reference Example 229

### ethyl (2E)-3-[1-methyl-5-(1H-pyrrolo[2,3-b]pyridin-1-yl)-3-(trifluoromethyl)-1H-pyrazol-4-yl]acrylate

By a method similar to that in Reference Example 12, the title compound was obtained from 1-methyl-5-(1H-pyrrolo[2,3-b]pyridin-1-yl)-3-(trifluoromethyl)-1H-pyrazole-4-carbaldehyde obtained in Reference Example 228 and ethyl (diethoxyphosphoryl)acetate.

¹H-NMR (300 MHz, CDCl₃) δ:1.22 (d, J=7.2 Hz, 3H), 3.70 (s, 3H), 4.12 (q, J=7.2 Hz, 2H), 5.59 (d, J=16.2 Hz, 1H), 6.83 (d, J=3.8 Hz, 1H), 7.19 (d, J=3.8 Hz, 1H), 7.25 (dd, J=8.1, 4.5 Hz, 1H), 7.39 (d, J=16.2 Hz, 1H), 8.05 (d, J=8.1 Hz, 1H), 8.36 (d, J=4.5 Hz, 1H).

### Reference Example 230

### (2E)-3-[1-methyl-5-(1H-pyrrolo[2,3-b]pyridin-1-yl)-3-(trifluoromethyl)-1H-pyrazol-4-yl]acrylic acid

By a method similar to that in Reference Example 13, the title compound was obtained from ethyl (2E)-3-[1-methyl-5-(1H-pyrrolo[2,3-b]pyridin-1-yl)-3-(trifluoromethyl)-1H-pyrazol-4-yl]acrylate obtained in Reference Example 229.

¹H-NMR (300 MHz, DMSO-d₆) δ:3.66 (s, 3H), 5.23 (d, J=16.5 Hz, 1H), 6.95 (d, J=3.8 Hz, 1H), 7.18 (d, J=16.5 Hz, 1H), 7.32 (dd, J=8.0, 4.5 Hz, 1H), 7.80 (d, J=3.8 Hz, 1H), 8.18-8.23 (m, 1H), 8.28-8.32 (m, 1H), 12.56 (s, 1H).

### Reference Example 231

### 3-(1-naphthyl)thiophene-2-carbaldehyde

To a mixture of 1-naphthylboronic acid (1.67 g), 3-bromothiophene-2-carbaldehyde (1.81 g), a 2.0M aqueous sodium carbonate solution (10.0 mL) and 1,2-dimethoxyethane (30 mL) was added tetrakis(triphenylphosphine)palladium(0) (0.43 g), and the reaction mixture was heated under reflux under nitrogen atmosphere for 5 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 95:5, v/v) to give the title compound (2.28 g, yield 98%) as a pale-yellow oil.

¹H-NMR (300 MHz, CDCl₃) δ:7.29 (d, J=4.9 Hz, 1H), 7.45-7.49 (m, 4H), 7.74 (d, J=8.0 Hz, 1H), 7.83 (dd, J=4.9, 1.5 Hz, 1H), 7.91-7.98 (m, 2H), 9.60 (s, 1H).

### Reference Example 232

### ethyl (2E)-3-[3-(1-naphthyl)-2-thienyl]acrylate

By a method similar to that in Reference Example 12, the title compound was obtained from 3-(1-naphthyl)thiophene-2-carbaldehyde obtained in Reference Example 231 and ethyl (diethoxyphosphoryl)acetate.

¹H-NMR (300 MHz, CDCl₃) δ:1.23 (d, J=7.1 Hz, 3H), 4.13 (q, J=7.1 Hz, 2H), 6.26 (d, J=15.6 Hz, 1H), 7.14 (d, J=5.1 Hz, 1H), 7.35 (dd, J=7.0, 1.1 Hz, 1H), 7.40-7.58 (m, 5H), 7.66 (d, J=8.3 Hz, 1H), 7.86-7.96 (m, 2H).

### Reference Example 233

### (2E)-3-[3-(1-naphthyl)-2-thienyl]acrylic acid

By a method similar to that in Reference Example 13, the title compound was obtained from ethyl (2E)-3-[3-(1-naphthyl)-2-thienyl]acrylate obtained in Reference Example 232.

¹H-NMR (300 MHz, DMSO-d₆) δ:6.18 (d, J=15.5 Hz, 1H), 7.18-7.27 (m, 2H), 7.41 (d, J=6.1 Hz, 1H), 7.47-7.68 (m, 4H), 7.89 (d, J=5.3 Hz, 1H), 8.00-8.08 (m, 2H), 12.33 (s, 1H).

### Reference Example 234

### ethyl (2E)-3-[5-(5-chloro-1H-pyrrolo[2,3-b]pyridin-1-yl)-1,3-dimethyl-1H-pyrazol-4-yl]acrylate

To a solution of ethyl (diethoxyphosphoryl)acetate (2.54 g) in tetrahydrofuran (20 mL), which was cooled at 0° C. in an ice bath, was added 60% sodium hydride (in oil, 459 mg) with stirring, and the mixture was stirred at 0° C. for 30 min. A solution of 5-(5-chloro-1H-pyrrolo[2,3-b]pyridin-1-yl)-1,3-dimethyl-1H-pyrazole-4-carbaldehyde obtained in Reference Example 182 (2.03 g) in tetrahydrofuran (25 mL) was added to this reaction mixture at 0° C., and the reaction mixture was stirred at 0° C. for 3 hr. The reaction mixture was concentrated under reduced pressure, water was added to the residue, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v) to give the title compound (2.54 g, yield 99%) as colorless crystals.

¹H-NMR (300 MHz, CDCl₃) δ:1.23 (t, J=7.2 Hz, 3H), 2.45 (s, 3H), 3.57 (s, 3H), 4.14 (q, J=7.2 Hz, 2H), 5.69 (d, J=16.3 Hz, 1H), 6.73 (d, J=3.8 Hz, 1H), 7.23-7.30 (m, 2H), 8.00 (d, J=2.3 Hz, 1H), 8.29 (d, J=2.3 Hz, 1H).

### Reference Example 235

### (2E)-3-[5-(5-chloro-1H-pyrrolo[2,3-b]pyridin-1-yl)-1,3-dimethyl-1H-pyrazol-4-yl]acrylic acid

To a solution of ethyl (2E)-3-[5-(5-chloro-1H-pyrrolo[2,3-b]pyridin-1-yl)-1,3-dimethyl-1H-pyrazol-4-yl]acrylate obtained in Reference Example 234 (2.50 g) in a mixed solvent of tetrahydrofuran (15 mL) and ethanol (15 mL) was added a 1N aqueous sodium hydroxide solution (15 mL), and the mixture was stirred with heating at 60° C. for 2 hr. The reaction mixture was allowed to cool to room temperature, neutralized with an aqueous solution (80 mL) of potassium hydrogensulfate (2.1 g), and extracted with ethyl acetate. The organic layer was washed with saturated brine, dried over anhydrous magnesium sulfate, and filtrated. The filtrate was concentrated, and the residue was crystallized from hexane-ethanol to give the title compound (2.18 g, yield 95%) as colorless crystals.

¹H-NMR (300 MHz, DMSO-d₆) δ:2.37 (s, 3H), 3.49 (s, 3H), 5.46 (d, J=16.3 Hz, 1H), 6.88 (d, J=3.6 Hz, 1H), 7.05 (d, J=16.3 Hz, 1H), 7.83 (d, J=3.6 Hz, 1H), 8.29-8.33 (m, 2H), 12.17 (s, 1H).

### Reference Example 236

### 5-(5-bromo-1H-pyrrolo[2,3-b]pyridin-1-yl)-1,3-dimethyl-1H-pyrazole-4-carbaldehyde

By a method similar to that in Reference Example 1, the title compound was obtained from 5-bromo-1H-pyrrolo[2,3-b]pyridine and 5-chloro-1,3-dimethyl-1H-pyrazole-4-carbaldehyde.

¹H-NMR (300 MHz, CDCl₃) δ:2.55 (s, 3H), 3.67 (s, 3H), 6.72 (d, J=3.6 Hz, 1H), 7.33 (d, J=3.6 Hz, 1H), 8.15 (d, J=2.1 Hz, 1H), 8.38 (d, J=2.1 Hz, 1H), 9.60 (s, 1H).

### Reference Example 237

### ethyl (2E)-3-[5-(5-bromo-1H-pyrrolo[2,3-b]pyridin-1-yl)-1,3-dimethyl-1H-pyrazol-4-yl]acrylate

By a method similar to that in Reference Example 12, the title compound was obtained from 5-(5-bromo-1H-pyrrolo[2,3-b]pyridin-1-yl)-1,3-dimethyl-1H-pyrazole-4-carbaldehyde obtained in Reference Example 236 and ethyl (diethoxyphosphoryl)acetate.

¹H-NMR (300 MHz, CDCl₃) δ:1.24 (t, J=7.2 Hz, 3H), 2.45 (s, 3H), 3.57 (s, 3H), 4.14 (q, J=7.2 Hz, 2H), 5.69 (d, J=16.5 Hz, 1H), 6.72 (d, J=3.6 Hz, 1H), 7.22 (d, J=3.6 Hz, 1H), 7.28 (d, J=16.5 Hz, 1H), 8.16 (d, J=2.3 Hz, 1H), 8.37 (d, J=2.3 Hz, 1H).

### Reference Example 238

### (2E)-3-[5-(5-bromo-1H-pyrrolo[2,3-b]pyridin-1-yl)-1,3-dimethyl-1H-pyrazol-4-yl]acrylic acid

By a method similar to that in Reference Example 13, the title compound was obtained from ethyl (2E)-3-[5-(5-bromo-1H-pyrrolo[2,3-b]pyridin-1-yl)-1,3-dimethyl-1H-pyrazol-4-yl]acrylate obtained in Reference Example 237.

¹H-NMR (300 MHz, DMSO-d₆) δ:2.36 (s, 3H), 3.49 (s, 3H), 5.46 (d, J=16.3 Hz, 1H), 6.87 (d, J=3.4 Hz, 1H), 7.05 (d, J=16.3 Hz, 1H), 7.81 (d, J=3.4 Hz, 1H), 8.36 (d, J=2.3 Hz, 1H), 8.44 (d, J=2.3 Hz, 1H), 12.17 (s, 1H).

### Reference Example 239

### 2-[5-(5-chloro-1H-indol-1-yl)-1,3-dimethyl-1H-pyrazol-4-yl]ethyl methanesulfonate

To a solution of 2-[5-(5-chloro-1H-indol-1-yl)-1,3-dimethyl-1H-pyrazol-4-yl]ethanol obtained in Reference Example 63 (630 mg) in tetrahydrofuran (10 mL) were added triethylamine (442 mg) and methanesulfonyl chloride (393 mg), and the mixture was stirred at room temperature for 4 hr. Water was added to the reaction mixture, and the mixture was extracted with ethyl acetate. The organic layer was washed with saturated brine, dried over anhydrous magnesium sulfate, and filtrated. The filtrate was concentrated to give the title compound (797 mg, yield 99%) as a colorless oil.

¹H-NMR (300 MHz, CDCl₃) δ:2.31 (s, 3H), 2.58-2.75 (m, 2H), 2.77 (s, 3H), 3.46 (s, 3H), 4.04 (t, J=6.6 Hz, 2H), 6.69 (d, J=3.2 Hz, 1H), 6.95 (d, J=8.7 Hz, 1H), 7.14 (d, J=3.2 Hz, 1H), 7.20 (dd, J=8.7, 1.9 Hz, 1H), 7.67 (d, J=1.9 Hz, 1H).

### Reference Example 240

### tert-butyl 4-{2-[5-(5-chloro-1H-indol-1-yl)-1,3-dimethyl-1H-pyrazol-4-yl]ethyl}-3-oxopiperazine-1-carboxylate

To a solution of tert-butyl 3-oxopiperazine-1-carboxylate (466 mg) in N,N-dimethylformamide (5 mL), which was cooled at 0° C. in an ice bath, was added 60% sodium hydride (in oil, 120 mg) with stirring, and the mixture was stirred at room temperature for 15 min. A solution of 2-[5-(5-chloro-1H-indol-1-yl)-1,3-dimethyl-1H-pyrazol-4-yl]ethyl methanesulfonate obtained in Reference Example 239 (642 mg) in N,N-dimethylformamide (5 mL) was added to this reaction mixture, and the reaction mixture was stirred at 60° C. for 12 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 10:90, v/v) to give the title compound (486 mg, yield 59%) as a colorless amorphous solid.

¹H-NMR (300 MHz, CDCl₃) δ:1.44 (s, 9H), 2.32 (s, 3H), 2.40-2.53 (m, 2H), 2.96-3.05 (m, 2H), 3.18-3.28 (m, 2H), 3.40-3.50 (m, 5H), 3.96 (s, 2H), 6.70 (d, J=3.2 Hz, 1H), 6.95 (d, J=8.7 Hz, 1H), 7.14 (d, J=3.2 Hz, 1H), 7.19 (dd, J=8.7, 2.1 Hz, 1H), 7.67 (d, J=2.1 Hz, 1H).

### Reference Example 241

### 2-{2-[5-(5-chloro-1H-indol-1-yl)-1,3-dimethyl-1H-pyrazol-4-yl]ethoxy}-1H-isoindole-1,3(2H)-dione

To a solution of 2-[5-(5-chloro-1H-indol-1-yl)-1,3-dimethyl-1H-pyrazol-4-yl]ethanol obtained in Reference Example 63 (2.06 g) in tetrahydrofuran (50 mL) were added N-hydroxyphthalimide (1.29 g) and triphenylphosphine (2.23 g), and then diethyl azodicarboxylate (40% toluene solution, 5.57 g) was added, and the mixture was stirred at room temperature for 15 hr. The reaction mixture was concentrated under reduced pressure, ethyl acetate was added to the residue, and the insoluble material was filtered off. The filtrate was concentrated, and the residue was subjected to silica gel column chromatography (hexane-ethyl acetate 50:50, v/v) to give the title compound (2.97 g, yield 96%) as a colorless amorphous solid.

¹H-NMR (300 MHz, CDCl₃) δ:2.32 (s, 3H), 2.64-2.85 (m, 2H), 3.47 (s, 3H), 4.02 (t, J=7.6 Hz, 2H), 6.61 (d, J=3.4 Hz, 1H), 6.95 (d, J=8.7 Hz, 1H), 7.09-7.17 (m, 2H), 7.54 (d, J=1.9 Hz, 1H), 7.72-7.80 (m, 4H).

### Reference Example 242

### 1-{4-[2-(aminooxy)ethyl]-1,3-dimethyl-1H-pyrazol-5-yl}-5-chloro-1H-indole

To a solution of 2-{2-[5-(5-chloro-1H-indol-1-yl)-1,3-dimethyl-1H-pyrazol-4-yl]ethoxy}-1H-isoindole-1,3(2H)-dione obtained in Reference Example 241 (1.26 g) in tetrahydrofuran (20 mL) was added a 35% aqueous hydrazine solution (2.91 g), and the mixture was stirred at room temperature for 3 hr. Water was added to the reaction mixture, and the mixture was extracted with ethyl acetate. The organic layer was washed with saturated brine, dried over anhydrous magnesium sulfate, and filtrated. The filtrate was concentrated to give the title compound (919 mg, yield 99%) as a colorless oil.

¹H-NMR (300 MHz, CDCl₃) δ: 2.30 (s, 3H), 2.40-2.60 (m, 2H), 3.44 (s, 3H), 3.51 (t, J=6.6 Hz, 2H), 5.04 (s, 2H), 6.64-6.69 (m, 1H), 6.94-6.99 (m, 1H), 7.12-7.22 (m, 2H), 7.63-7.67 (m, 1H).

### Reference Example 243

### (4R)-5-{2-[5-(5-chloro-1H-indol-1-yl)-1,3-dimethyl-1H-pyrazol-4-yl]ethyl}-4-isopropyl-2-(4-methoxybenzyl)-1,2,5-thiadiazolidin-3-one 1,1-dioxide

To a solution of (4R)-4-isopropyl-2-(4-methoxybenzyl)-1,2,5-thiadiazolidin-3-one 1,1-dioxide obtained in Reference Example 273 (800 mg) in N,N-dimethylformamide (6 mL) was added 60% sodium hydride (in oil, 103 mg) with stirring at 0° C., and the mixture was stirred at room temperature for 10 min. A solution of 2-[5-(5-chloro-1H-indol-1-yl)-1,3-dimethyl-1H-pyrazol-4-yl]ethyl methanesulfonate obtained in Reference Example 239 (783 mg) in N,N-dimethylformamide (6 mL) was added to this reaction mixture, and the reaction mixture was stirred at 100° C. for 5 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 60:40, v/v) to give the title compound (740 mg, yield 59%) as a colorless amorphous solid.

¹H-NMR (300 MHz, CDCl₃) δ:0.58 (dd, J=17.6, 7.0 Hz, 3H), 0.79 (dd, J=7.0, 2.7 Hz, 3H), 1.61-1.82 (m, 1H), 2.30 (s, 3H), 2.50-2.88 (m, 3H), 3.08-3.32 (m, 1H), 3.41 (d, J=3.0 Hz, 1H), 3.51 (d, J=3.0 Hz, 3H), 3.78 (s, 3H), 4.53-4.66 (m, 2H), 6.67-6.70 (m, 1H), 6.83 (d, J=8.3 Hz, 2H), 6.89-6.93 (m, 1H), 7.08 (d, J=3.4 Hz, 1H), 7.14-7.22 (m, 1H), 7.31 (d, J=8.3 Hz, 2H), 7.66 (s, 1H).

### Reference Example 244

### 5-(5-chloro-1H-pyrrolo[2,3-b]pyridin-1-yl)-3-cyclopropyl-1-methyl-1H-pyrazole-4-carbaldehyde

To a solution of 5-chloro-1H-pyrrolo[2,3-b]pyridine (1.30 g) in N,N-dimethylformamide (25 mL), which was cooled at 0° C. in an ice bath, was added 60% sodium hydride (in oil, 380 mg) with stirring, and the mixture was stirred at 0° C. for 20 min. 5-Chloro-3-cyclopropyl-1-methyl-1H-pyrazole-4-carbaldehyde obtained in Reference Example 150 (1.47 g) was added to this reaction mixture at 0° C., and the reaction mixture was stirred at 100° C. for 4 hr. After the reaction mixture was allowed to cool to room temperature, water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30, v/v) to give the title compound (1.69 g, yield 70%) as colorless crystals.

¹H-NMR (300 MHz, CDCl₃) δ:1.00-1.08 (m, 4H), 2.40-2.52 (m, 1H), 3.61 (s, 3H), 6.72 (d, J=3.6 Hz, 1H), 7.34 (d, J=3.6 Hz, 1H), 7.99 (d, J=2.3 Hz, 1H), 8.29 (d, J=2.3 Hz, 1H), 9.68 (s, 1H).

### Reference Example 245

### (E)-2-[5-(5-chloro-1H-pyrrolo[2,3-b]pyridin-1-yl)-3-cyclopropyl-1-methyl-1H-pyrazol-4-yl]ethylenesulfonamide

To a solution of tert-butyl {[(diphenylphosphoryl)methyl]sulfonyl}carbamate (2.64 g) in N,N-dimethylformamide (20 mL) was added 60% sodium hydride (in oil, 676 mg) with stirring at 0° C., and the mixture was stirred at 0° C. for 1 hr. 5-(5-Chloro-1H-pyrrolo[2,3-b]pyridin-1-yl)-3-cyclopropyl-1-methyl-1H-pyrazole-4-carbaldehyde obtained in Reference Example 244 (1.68 g) was added to the reaction mixture at 0° C., and the mixture was stirred at room temperature for 1.5 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v) to give colorless crystals. Trifluoroacetic acid (20 mL) was added to the colorless crystals, and the mixture was stirred at room temperature for 3 hr. The reaction mixture was concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v), and crystallized from hexane-ethyl acetate to give the title compound (1.49 g, yield 74%) as colorless crystals.

¹H-NMR (300 MHz, CDCl₃) δ:1.01 (d, J=7.6 Hz, 4H), 1.88-1.96 (m, 1H), 3.55 (s, 3H), 4.48 (s, 2H), 6.34 (d, J=15.5 Hz, 1H), 6.74 (d, J=3.8 Hz, 1H), 7.20-7.26 (m, 2H), 8.01 (d, J=2.3 Hz, 1H), 8.29 (d, J=2.3 Hz, 1H).

### Reference Example 246

### 2-[5-(5-chloro-1H-pyrrolo[2,3-b]pyridin-1-yl)-3-cyclopropyl-1-methyl-1H-pyrazol-4-yl]ethanesulfonamide

To a solution of (E)-2-[5-(5-chloro-1H-pyrrolo[2,3-b]pyridin-1-yl)-3-cyclopropyl-1-methyl-1H-pyrazol-4-yl]ethylenesulfonamide obtained in Reference Example 245 (935 mg) in a mixed solvent of tetrahydrofuran (25 mL) and ethanol (25 mL) was added 10% palladium carbon (200 mg), and the mixture was stirred under 1 atom of hydrogen atmosphere at room temperature for 3 hr. The catalyst was removed by filtration, and the filtrate was concentrated. The residue was subjected to silica gel column chromatography (hexane-ethyl acetate 30:70, v/v), and crystallized from hexane-ethyl acetate to give the title compound (758 mg, yield 80%) as colorless crystals.

¹H-NMR (300 MHz, CDCl₃) δ:0.92-1.00 (m, 4H), 1.78-1.89 (m, 1H), 2.78-2.90 (m, 1H), 2.95-3.25 (m, 2H), 3.30-3.40 (m, 1H), 3.42 (s, 3H), 4.83 (s, 2H), 6.69 (d, J=3.4 Hz, 1H), 7.22 (d, J=3.4 Hz, 1H), 8.00 (d, J=2.3 Hz, 1H), 8.25 (d, J=2.3 Hz, 1H).

### Reference Example 247

### 5-{2-[5-(5-chloro-1H-indol-1-yl)-1,3-dimethyl-1H-pyrazol-4-yl]ethyl}-2-(4-methoxybenzyl)-1,2,5-thiadiazolidin-3-one 1,1-dioxide

By a method similar to that in Reference Example 243, the title compound was obtained from 2-[5-(5-chloro-1H-indol-1-yl)-1,3-dimethyl-1H-pyrazol-4-yl]ethyl methanesulfonate obtained in Reference Example 239 and 2-(4-methoxybenzyl)-1,2,5-thiadiazolidin-3-one 1,1-dioxide obtained in Reference Example 269.

¹H-NMR (300 MHz, CDCl₃) δ:2.30 (s, 3H), 2.44-2.63 (m, 2H), 3.02 (t, J=6.8 Hz, 2H), 3.19-3.34 (m, 2H), 3.47 (s, 3H), 3.79 (s, 3H), 4.59 (s, 2H), 6.62 (d, J=3.2 Hz, 1H), 6.86 (d, J=8.3 Hz, 2H), 6.93 (d, J=8.7 Hz, 1H), 7.06 (d, J=3.2 Hz, 1H), 7.11 (dd, J=8.7, 1.7 Hz, 1H), 7.33 (d, J=8.3 Hz, 2H), 7.66 (s, 1H).

### Reference Example 248

### 1-benzyl-5-chloro-3-methyl-1H-pyrazole-4-carbaldehyde

By a method similar to that in Reference Example 85, the title compound was obtained from 2-benzyl-5-methyl-2,4-dihydro-3H-pyrazol-3-one.

¹H-NMR (300 MHz, CDCl₃) δ:2.47 (s, 3H), 5.30 (s, 2H), 7.23-7.38 (m, 5H), 9.87 (s, 1H).

### Reference Example 249

### 1-benzyl-3-methyl-5-(1H-pyrrolo[2,3-b]pyridin-1-yl)-1H-pyrazole-4-carbaldehyde

By a method similar to that in Reference Example 1, the title compound was obtained from 1H-pyrrolo[2,3-b]pyridine and 1-benzyl-5-chloro-3-methyl-1H-pyrazole-4-carbaldehyde obtained in Reference Example 248.

¹H-NMR (300 MHz, CDCl₃) δ:2.57 (s, 3H), 5.16 (s, 2H), 6.66 (d, J=3.8 Hz, 1H), 6.85-6.92 (m, 2H), 7.04 (d, J=3.8 Hz, 1H), 7.15-7.27 (m, 4H), 7.98-8.01 (m, 1H), 8.35 (d, J=4.9 Hz, 1H), 9.55 (s, 1H).

### Reference Example 250

### ethyl (2E)-3-[1-benzyl-3-methyl-5-(1H-pyrrolo[2,3-b]pyridin-1-yl)-1H-pyrazol-4-yl]acrylate

By a method similar to that in Reference Example 12, the title compound was obtained from 1-benzyl-3-methyl-5-(1H-pyrrolo[2,3-b]pyridin-1-yl)-1H-pyrazole-4-carbaldehyde obtained in Reference Example 249 and ethyl (diethoxyphosphoryl)acetate.

¹H-NMR (300 MHz, CDCl₃) δ:1.21 (t, J=7.1 Hz, 3H), 2.47 (s, 3H), 4.11 (q, J=7.1 Hz, 2H), 4.95 (d, J=15.0 Hz, 1H), 5.15 (d, J=15.0 Hz, 1H), 5.68 (d, J=16.2 Hz, 1H), 6.64 (d, J=3.6 Hz, 1H), 6.82-6.95 (m, 3H), 7.12-7.36 (m, 5H), 7.97-8.00 (m, 1H), 8.33 (dd, J=4.9, 1.5 Hz, 1H).

### Reference Example 251

### (2E)-3-[1-benzyl-3-methyl-5-(1H-pyrrolo[2,3-b]pyridin-1-yl)-1H-pyrazol-4-yl]acrylic acid

By a method similar to that in Reference Example 13, the title compound was obtained from ethyl (2E)-3-[1-benzyl-3-methyl-5-(1H-pyrrolo[2,3-b]pyridin-1-yl)-1H-pyrazol-4-yl]acrylate obtained in Reference Example 250.

¹H-NMR (300 MHz, DMSO-d₆) δ: 2.38 (s, 3H), 4.83 (d, J=15.6 Hz, 1H), 5.06 (d, J=15.6 Hz, 1H), 5.47 (d, J=16.3 Hz, 1H), 6.83 (d, J=3.4 Hz, 1H), 6.93 (dd, J=5.7, 4.2 Hz, 2.H), 7.05 (d, J=16.3 Hz, 1H), 7.16-7.22 (m, 3H), 7.26 (dd, J=8.0, 4.5 Hz, 1H), 7.58 (d, J=3.4 Hz, 1H), 8.14 (dd, J=8.0, 1.5 Hz, 1H), 8.22-8.31 (m, 1H), 12.15 (s, 1H).

### Reference Example 252

### 3-methyl-5-(1H-pyrrolo[2,3-b]pyridin-1-yl)-1H-pyrazole-4-carbaldehyde

To 1-benzyl-3-methyl-5-(1H-pyrrolo[2,3-b]pyridin-1-yl)-1H-pyrazole-4-carbaldehyde obtained in Reference Example 249 (2.32 g) was added trifluoroacetic acid (35 mL), and the mixture was heated under reflux for 150 hr. The reaction mixture was concentrated under reduced pressure, water was added to the residue, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 55:45, v/v) to give the title compound (880 mg, yield 53%) as colorless crystals.

¹H-NMR (300 MHz, CDCl₃) δ:2.52 (s, 3H), 6.68 (d, J=3.8 Hz, 1H), 7.22 (dd, J=7.6, 4.9 Hz, 1H), 7.98-8.14 (m, 2H), 8.36 (d, J=3.8 Hz, 1H), 9.88 (s, 1H).

### Reference Example 253

### ethyl (2E)-3-[3-methyl-5-(1H-pyrrolo[2,3-b]pyridin-1-yl)-1H-pyrazol-4-yl]acrylate

To a solution of 3-methyl-5-(1H-pyrrolo[2,3-b]pyridin-1-yl)-1H-pyrazole-4-carbaldehyde obtained in Reference Example 252 (473 mg) in toluene (20 mL) was added ethyl (triphenylphosphoranylidene)acetate (1.16 g), and the mixture was heated under reflux for 15 hr. The reaction mixture was concentrated under reduced pressure, and the residue was subjected to silica gel column chromatography (hexane-ethyl acetate 10:90, v/v) to give the title compound (589 mg, yield 95%) as colorless crystals.

¹H-NMR (300 MHz, CDCl₃) δ: 1.23 (t, J=7.2 Hz, 3H), 2.32 (s, 3H), 4.14 (q, J=7.2 Hz, 2H), 5.56-5.62 (m, 1H), 6.70 (d, J=3.4 Hz, 1H), 7.18 (dd, J=7.8, 4.6 Hz, 1H), 7.37 (d, J=3.4 Hz, 1H), 7.41-7.49 (m, 1H), 8.03 (dd, J=7.8, 1.5 Hz, 1H), 8.35 (dd, J=4.6, 1.5 Hz, 1H), 12.00 (s, 1H).

### Reference Example 254

### (2E)-3-[3-methyl-5-(1H-pyrrolo[2,3-b]pyridin-1-yl)-1H-pyrazol-4-yl]acrylic acid

To a solution of ethyl (2E)-3-[3-methyl-5-(1H-pyrrolo[2,3-b]pyridin-1-yl)-1H-pyrazol-4-yl]acrylate obtained in Reference Example 253 (1.75 g) in a mixed solvent of tetrahydrofuran (12 mL) and ethanol (12 mL) was added a 1N aqueous sodium hydroxide solution (18 mL), and the mixture was stirred with heating at 80° C. for 4 hr. The reaction mixture was allowed to cool to room temperature, neutralized with an aqueous solution (50 mL) of potassium hydrogensulfate (2.5 g), and extracted with ethyl acetate. The organic layer was washed with saturated brine, dried over anhydrous magnesium sulfate, and filtrated. The filtrate was concentrated, and the residue was crystallized from hexane-ethanol to give the title compound (1.10 g, yield 69%) as colorless crystals.

¹H-NMR (300 MHz, DMSO-d₆) δ:2.46 (s, 3H), 5.34 (d, J=16.2 Hz, 1H), 6.73 (d, J=3.6 Hz, 1H), 7.16-7.28 (m, 2H), 7.60 (d, J=3.6 Hz, 1H), 8.09 (dd, J=7.8, 1.6 Hz, 1H), 8.22 (dd, J=4.7, 1.5 Hz, 1H), 12.00 (s, 1H), 13.23 (s, 1H).

### Reference Example 255

### (2E)-3-[1-(tert-butoxycarbonyl)-5-methyl-3-(1H-pyrrolo[2,3-b]pyridin-1-yl)-1H-pyrazol-4-yl]acrylic acid

To a solution of (2E)-3-[3-methyl-5-(1H-pyrrolo[2,3-b]pyridin-1-yl)-1H-pyrazol-4-yl]acrylic acid obtained in Reference Example 254 (715 mg) in a mixed solvent of tetrahydrofuran (10 mL) and water (5 mL) were added sodium carbonate (284 mg) and di-tert-butyl dicarbonate (6.24 g), and the mixture was stirred at room temperature for 72 hr. The reaction mixture was neutralized with potassium hydrogensulfate (0.73 g), and extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v) to give the title compound (549 mg, yield 56%) as colorless crystals.

¹H-NMR (300 MHz, DMSO-d₆) δ:1.60 (s, 9H), 2.69 (s, 3H), 5.11 (d, J=16.2 Hz, 1H), 6.80 (d, J=3.6 Hz, 1H), 7.23 (dd, J=7.8, 4.6 Hz, 1H), 7.33 (d, J=16.2 Hz, 1H), 7.68 (d, J=3.6 Hz, 1H), 8.13 (dd, J=7.8, 1.5 Hz, 1H), 8.24 (dd, J=4.6, 1.5 Hz, 1H), 12.28 (s, 1H).

### Reference Example 256

### 1-benzyl-2-butyl-4-(1-naphthyl)-1H-imidazole-5-carbaldehyde

By a method similar to that in Reference Example 9, the title compound was obtained from 1-benzyl-2-butyl-4-chloro-1H-imidazole-5-carbaldehyde and 1-naphthylboronic acid.

¹H-NMR (300 MHz, CDCl₃) δ:0.91 (t, J=7.3 Hz, 3H), 1.35-1.48 (m, 2H), 1.70-1.84 (m, 2H), 2.75-2.81 (m, 2H), 5.72 (s, 2H), 7.15 (d, J=7.0 Hz, 2H), 7.29-7.40 (m, 3H), 7.49-7.60 (m, 4H), 7.87-7.95 (m, 2H), 8.09-8.15 (m, 1H), 9.50 (s, 1H).

### Reference Example 257

### ethyl (2E)-3-[1-benzyl-2-butyl-4-(1-naphthyl)-1H-imidazol-5-yl]acrylate

By a method similar to that in Reference Example 12, the title compound was obtained from 1-benzyl-2-butyl-4-(1-naphthyl)-1H-imidazole-5-carbaldehyde obtained in Reference Example 256 and ethyl (diethoxyphosphoryl)acetate.

¹H-NMR (300 MHz, CDCl₃) δ:0.89 (t, J=7.4 Hz, 3H), 1.11 (t, J=7.0 Hz, 3H), 1.34-1.46 (m, 2H), 1.70-1.82 (m, 2H), 2.73-2.79 (m, 2H), 4.01 (q, J=7.0 Hz, 2H), 5.34 (s, 2H), 5.56 (d, J=16.3 Hz, 1H), 7.11 (d, J=6.8 Hz, 2H), 7.30-7.56 (m, 8H), 7.85-7.93 (m, 3H).

### Reference Example 258

### (2E)-3-[1-benzyl-2-butyl-4-(1-naphthyl)-1H-imidazol-5-yl]acrylic acid

By a method similar to that in Reference Example 13, the title compound was obtained from ethyl (2E)-3-[1-benzyl-2-butyl-4-(1-naphthyl)-1H-imidazol-5-yl]acrylate obtained in Reference Example 257.

¹H-NMR (300 MHz, DMSO-d₆) δ: 0.83 (t, J=7.4 Hz, 3H), 1.28-1.42 (m, 2H), 1.58-1.69 (m, 2H), 2.77 (t, J=7.6 Hz, 2H), 5.39 (d, J=16.3 Hz, 1H), 5.51 (s, 2H), 7.11 (d, J=7.2 Hz, 2H), 7.28 (d, J=16.3 Hz, 1H), 7.34 (d, J=7.6 Hz, 1H), 7.39-7.64 (m, 6H), 7.81 (d, J=8.3 Hz, 1H), 8.02 (d, J=8.3 Hz, 2H), 12.02 (s, 1H).

### Reference Example 259

### ethyl N-({[(benzyloxy)carbonyl]amino}sulfonyl)-β-alaninate

To a solution of benzyl alcohol (2.25 g) in acetonitrile (40 ml) was added chlorosulfonyl isocyanate (1.90 mL) with stirring at 0° C., and the mixture was stirred for 30 min. Pyridine (3.35 mL) was added to this reaction mixture, and the mixture was stirred at 0° C. for 1 hr. β-Alanine ethyl ester hydrochloride (4.79 g) and N,N-diisopropylethylamine (7.13 mL) were added, and the mixture was stirred at room temperature for 3 hr. 1N Hydrochloric acid was added to this reaction mixture, and the mixture was extracted with ethyl acetate. The organic layer was washed with 1N hydrochloric acid and saturated brine, dried over anhydrous magnesium sulfate, and filtrated. The filtrate was concentrated, and the residue was crystallized from hexane-ethyl acetate to give the title compound (6.55 g, yield 95%) as colorless crystals.

¹H-NMR (300 MHz, CDCl₃) δ:1.27 (t, J=7.2 Hz, 3H), 2.59 (t, J=6.1 Hz, 2H), 3.24-3.45 (m, 2H), 4.16 (q, J=7.2 Hz, 2H), 5.20 (s, 2H), 5.76 (t, J=6.2 Hz, 1H), 7.30-7.38 (m, 5H), 7.40 (br s, 1H).

### Reference Example 260

### ethyl N-(aminosulfonyl)-β-alaninate

By a method similar to that in Reference Example 109, the title compound was obtained from ethyl N-({[(benzyloxy)carbonyl]amino}sulfonyl)-β-alaninate obtained in Reference Example 259.

¹H-NMR (300 MHz, CDCl₃) δ:1.28 (t, J=7.2 Hz, 3H), 2.65 (t, J=5.9 Hz, 2H), 3.28-3.54 (m, 2H), 4.17 (q, J=7.2 Hz, 2H), 4.61 (br s, 2H), 5.05 (t, J=5.7 Hz, 1H).

### Reference Example 261

### 5-(difluoromethyl)-2-methyl-2,4-dihydro-3H-pyrazol-3-one

By a method similar to that in Reference Example 84, the title compound was obtained from ethyl 4,4-difluoro-3-oxobutanoate and methylhydrazine.

¹H-NMR (300 MHz, DMSO-d₆) δ:3.54 (s, 3H), 5.55 (s, 1H), 6.70 (t, J=54.8 Hz, 1H), 11.35 (s, 1H).

### Reference Example 262

### 5-chloro-3-(difluoromethyl)-1-methyl-1H-pyrazole-4-carbaldehyde

By a method similar to that in Reference Example 85, the title compound was obtained from 5-(difluoromethyl)-2-methyl-2,4-dihydro-3H-pyrazol-3-one obtained in Reference Example 261.

¹H-NMR (300 MHz, CDCl₃) δ:3.93 (s, 3H), 6.90 (t, J=53.6 Hz, 1H), 9.96 (s, 1H).

### Reference Example 263

### 3-(difluoromethyl)-1-methyl-5-(1H-pyrrolo[2,3-b]pyridin-1-yl)-1H-pyrazole-4-carbaldehyde

By a method similar to that in Reference Example 1, the title compound was obtained from 1H-pyrrolo[2,3-b]pyridine and 5-chloro-3-(difluoromethyl)-1-methyl-1H-pyrazole-4-carbaldehyde obtained in Reference Example 262.

¹H-NMR (300 MHz, CDCl₃) δ:3.81 (s, 3H), 6.77-7.16 (m, 2H), 7.24 (dd, J=8.0, 4.7 Hz, 1H), 7.34 (d, J=3.8 Hz, 1H), 8.03 (dd, J=8.0, 1.5 Hz, 1H), 8.35 (dd, J=4.7, 1.5 Hz, 1H), 9.79 (s, 1H).

### Reference Example 264

### ethyl (2E)-3-[3-(difluoromethyl)-1-methyl-5-(1H-pyrrolo[2,3-b]pyridin-1-yl)-1H-pyrazol-4-yl]acrylate

By a method similar to that in Reference Example 12, the title compound was obtained from 3-(difluoromethyl)-1-methyl-5-(1H-pyrrolo[2,3-b]pyridin-1-yl)-1H-pyrazole-4-carbaldehyde obtained in Reference Example 263 and ethyl (diethoxyphosphoryl)acetate.

¹H-NMR (300 MHz, CDCl₃) δ:1.24 (d, J=7.2 Hz, 3H), 3.67 (s, 3H), 4.13 (q, J=7.2 Hz, 2H), 5.86 (d, J=16.3 Hz, 1H), 6.58-6.93 (m, 2H), 7.20-7.29 (m, 2H), 7.37 (d, J=16.3 Hz, 1H), 8.05 (dd, J=8.0, 1.5 Hz, 1H), 8.36 (dd, J=4.9, 1.5 Hz, 1H).

### Reference Example 265

### (2E)-3-[3-(difluoromethyl)-1-methyl-5-(1H-pyrrolo[2,3-b]pyridin-1-yl)-1H-pyrazol-4-yl]acrylic acid

By a method similar to that in Reference Example 13, the title compound was obtained from ethyl (2E)-3-[3-(difluoromethyl)-1-methyl-5-(1H-pyrrolo[2,3-b]pyridin-1-yl)-1H-pyrazol-4-yl]acrylate obtained in Reference Example 264.

¹H-NMR (300 MHz, DMSO-d₆) δ:3.62 (s, 3H), 5.42 (d, J=16.3 Hz, 1H), 6.94 (d, J=3.6 Hz, 1H), 7.06-7.42 (m, 3H), 7.81 (d, J=3.6 Hz, 1H), 8.20 (dd, J=8.0, 1.5 Hz, 1H), 8.30 (dd, J=4.5, 1.5 Hz, 1H), 12.41 (s, 1H).

### Reference Example 266

### ethyl N-{[(tert-butoxycarbonyl)amino]sulfonyl}glycinate

To a solution of tert-butyl alcohol (10 g) in acetonitrile (200 mL), which was cooled at 0° C. in an ice bath, was added dropwise chlorosulfonyl isocyanate (22.9 g), and the mixture was stirred at 0° C. for 1 hr. Pyridine (33 mL) was added to the reaction mixture at 0° C., and the reaction mixture was further stirred at 0° C. for 45 min to give a solution of tert-butyl N-chlorosulfonyl carbamate in acetonitrile. To a suspension of glycine ethyl ester hydrochloride (56.5 g) in acetonitrile (200 mL), which was cooled at 0° C. in an ice bath, was added triethylamine (57 mL), and the mixture was stirred at 0° C. for 20 min. The white precipitate was removed by filtration, and washed with a small amount of acetonitrile. The obtained filtrate was added to the aforementioned solution of tert-butyl N-chlorosulfonyl carbamate in acetonitrile, which was cooled at 0° C. in an ice bath, and the mixture was stirred at room temperature for 14 hr. The reaction mixture was concentrated under reduced pressure, 1M hydrochloric acid (260 mL) was added to the residue, and the mixture was extracted with ethyl acetate. The organic layer was washed with saturated brine, dried over anhydrous magnesium sulfate, and filtrated. The filtrate was concentrated, and the residue was suspended in dichloromethane (100 mL) heated in advance, and the insoluble material was collected by filtration, and dried to give the title compound (30.3 g, yield 80%) as colorless crystals.

¹H-NMR (300 MHz, CDCl₃) δ:1.30 (t, J=7.1 Hz, 3H), 1.50 (s, 9H), 3.95 (s, 2H), 4.23 (q, J=7.2 Hz, 2H), 5.63 (br s, 1H).

### Reference Example 267

### ethyl N-{[(tert-butoxycarbonyl)(4-methoxybenzyl)amino]sulfonyl}glycinate

To a solution of ethyl N-{[(tert-butoxycarbonyl)amino]sulfonyl}glycinate obtained in Reference Example 266 (20.0 g), triphenylphosphine (18.6 g) and 4-methoxybenzyl alcohol (9.79 g) in tetrahydrofuran (100 mL) were added diethyl azodicarboxylate (31.6 g) and tetrahydrofuran (20 mL) under nitrogen atmosphere at 0° C., and the mixture was stirred at room temperature for 24 hr. The reaction mixture was concentrated under reduced pressure, a saturated aqueous sodium hydrogencarbonate solution was added to the residue, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 75:25, v/v), and crystallized from hexane-isopropyl ether to give the title compound (17.7 g, yield 62%) as colorless crystals.

¹H-NMR (300 MHz, CDCl₃) δ:1.25 (t, J=7.1 Hz, 3H), 1.54 (s, 9H), 3.55 (d, J=5.4 Hz, 2H), 3.80 (s, 3H), 4.14 (q, J=7.1 Hz, 2H), 4.76 (s, 2H), 5.70 (t, J=5.4 Hz, 1H), 6.84 (d, J=9.0 Hz, 2H), 7.32 (d, J=8.7 Hz, 2H).

### Reference Example 268

### ethyl N-{[(4-methoxybenzyl)amino]sulfonyl}glycinate

To ethyl N-{[(tert-butoxycarbonyl)(4-methoxybenzyl)amino]sulfonyl}glycinate obtained in Reference Example 267 (10.0 g) was added a 4M hydrogen chloride-ethyl acetate solution (100 mL) at 0° C., and the mixture was stirred at 0° C. for 1 hr, and then at room temperature for 3.5 hr. The reaction mixture was concentrated under reduced pressure, the residue was subjected to silica gel column chromatography (hexane-ethyl acetate 80:20-60:40, v/v) to give the title compound (6.48 g, yield 86%) as a white solid.

¹H-NMR (300 MHz, CDCl₃) δ:1.28 (t, J=7.2 Hz, 3H), 3.79 (d, J=5.1 Hz, 2H), 3.80 (s, 3H), 4.17-4.25 (m, 4H), 4.52 (t, J=5.9 Hz, 1H), 4.82 (t, J=5.6 Hz, 1H), 6.86 (d, J=8.7 Hz, 2H), 7.24 (d, J=8.7 Hz, 2H).

### Reference Example 269

### 2-(4-methoxybenzyl)-1,2,5-thiadiazolidin-3-one 1,1-dioxide

To a solution of ethyl N-{[(4-methoxybenzyl)amino]sulfonyl}glycinate obtained in Reference Example 268 (6.21 g) in methanol (60 mL) were added sodium methoxide (about 28% methanol solution: 11.9 g) and methanol (40 mL), and the mixture was stirred at room temperature for 6 hr. 1M Hydrochloric acid (70 mL) was added to the reaction mixture at 0° C., and the reaction mixture was concentrated under reduced pressure. Water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30-60:40, v/v), and crystallized from hexane-ethyl acetate to give the title compound (3.76 g, yield 71%) as colorless crystals.

¹H-NMR (300 MHz, CDCl₃) δ:3.79 (s, 3H), 4.02 (d, J=7.2 Hz, 2H), 4.68 (s, 2H), 4.83 (br s, 1H), 6.86 (d, J=8.4 Hz, 2H), 7.35 (d, J=8.4 Hz, 2H).

### Reference Example 270

### methyl N-{[(tert-butoxycarbonyl)amino]sulfonyl}-D-valinate

By a method similar to that in Reference Example 266, the title compound was obtained from D-valine methyl ester hydrochloride, tert-butyl alcohol and chlorosulfonyl isocyanate.

¹H-NMR (300 MHz, CDCl₃) δ:0.91 (d, J=6.6 Hz, 3H), 1.01 (d, J=6.6 Hz, 3H), 1.49 (s, 9H), 2.09-2.19 (m, 1H), 3.75 (s, 3H), 4.03 (dd, J=9.3, 4.8 Hz, 1H), 5.63 (d, J=9.3 Hz, 1H).

### Reference Example 271

### methyl N-{[(tert-butoxycarbonyl)(4-methoxybenzyl)amino]sulfonyl}-D-valinate

By a method similar to that in Reference Example 267, the title compound was obtained from methyl N-{[(tert-butoxycarbonyl)amino]sulfonyl}-D-valinate obtained in Reference Example 270 and 4-methoxybenzyl alcohol.

¹H-NMR (300 MHz, CDCl₃) δ:0.82 (d, J=6.9 Hz, 3H), 0.93 (d, J=6.6 Hz, 3H), 1.53 (s, 9H), 1.97-2.04 (m, 1H), 3.58 (dd, J=8.7, 4.8 Hz, 1H), 3.62 (s, 3H), 3.80 (s, 3H), 4.64 (d, J=15.3 Hz, 1H), 4.81 (d, J=15.3 Hz, 1H), 5.78 (d, J=8.7 Hz, 1H), 6.84 (d, J=8.7 Hz, 2H), 7.30 (d, J=9.0 Hz, 2H).

### Reference Example 272

### methyl N-{[(4-methoxybenzyl)amino]sulfonyl}-D-valinate

By a method similar to that in Reference Example 268, the title compound was obtained from methyl N-{[(tert-butoxycarbonyl)(4-methoxybenzyl)amino]sulfonyl}-D-valinate obtained in Reference Example 271.

¹H-NMR (300 MHz, CDCl₃) δ:0.91 (d, J=6.9 Hz, 3H), 1.02 (d, J=6.6 Hz, 3H), 2.06-2.16 (m, 1H), 3.74 (s, 3H), 3.80 (s, 3H), 3.84 (dd, J=9.9, 4.8 Hz, 1H), 4.08-4.15 (m, 2H), 4.32 (t, J=6.0 Hz, 1H), 4.97 (d, J=9.9 Hz, 1H), 6.86 (d, J=8.4 Hz, 2H), 7.22 (d, J=8.4 Hz, 2H).

### Reference Example 273

### (4R)-4-isopropyl-2-(4-methoxybenzyl)-1,2,5-thiadiazolidin-3-one 1,1-dioxide

By a method similar to that in Reference Example 269, the title compound was obtained from methyl N-{[(4-methoxybenzyl)amino]sulfonyl}-D-valinate obtained in Reference Example 272.

¹H-NMR (300 MHz, CDCl₃) δ:0.92 (d, J=6.9 Hz, 3H), 1.04 (d, J=6.9 Hz, 3H), 2.30-2.40 (m, 1H), 3.79 (s, 3H), 4.07 (dd, J=6.9, 3.6 Hz, 1H), 4.64 (d, J=15.0 Hz, 1H), 4.69 (d, J=15.0 Hz, 1H), 4.75 (d, J=6.9 Hz, 1H), 6.85 (d, J=8.7 Hz, 2H), 7.35 (d, J=8.7 Hz, 2H).

### Reference Example 274

### methyl 2,5-dimethyl-4-(1-naphthyl)-1-{[2-(trimethylsilyl)ethoxy]methyl}-1H-pyrrole-3-carboxylate

To a solution of methyl 4-bromo-2,5-dimethyl-1-{[2-(trimethylsilyl)ethoxy]methyl}-1H-pyrrole-3-carboxylate (827 mg) in toluene (22 mL) were added 1-naphthaleneboronic acid (785 mg) and potassium carbonate (1.89 g) under argon atmosphere, and the mixture was stirred at room temperature for 30 min. Tris(dibenzylideneacetone)dipalladium(0) (52 mg) and 2-dicyclohexylphosphino-2′,6′-dimethoxybiphenyl (93 mg) were added to this reaction mixture, and the mixture was stirred at 100° C. for 18 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 90:10, v/v) to give the title compound (902 mg, yield 96%) as a colorless oil.

¹H-NMR (300 MHz, CDCl₃) δ:0.00 (s, 9H), 0.90-0.97 (m, 2H), 1.98 (s, 3H), 2.67 (s, 3H), 3.23 (s, 3H), 3.53-3.60 (m, 2H), 5.09-5.38 (m, 2H), 7.22-7.48 (m, 5H), 7.74-7.85 (m, 2H).

### Reference Example 275

### 2,5-dimethyl-4-(1-naphthyl)-1-{[2-(trimethylsilyl)ethoxy]methyl}-1H-pyrrole-3-carbaldehyde

To a solution of methyl 2,5-dimethyl-4-(1-naphthyl)-1-{[2-(trimethylsilyl)ethoxy]methyl}-1H-pyrrole-3-carboxylate obtained in Reference Example 274 (1.22 g) in diethyl ether (30 mL) was added diisobutylaluminum hydride (1.5M toluene solution, 4.9 mL) with stirred at 0° C., and the mixture was stirred at room temperature for 2 hr. Methanol and water were added to this reaction mixture, the mixture was filtrated through Celite®, and the filtrate was concentrated. The residue was dissolved in dichloromethane (10 mL), the solution was added to a mixture of pyridinium dichromate (1.34 g) and Celite® (1.34 g) in dichloromethane (30 mL) with stirring, and the mixture was stirred at room temperature for 7 hr. The reaction mixture was filtrated through Celite®, and the filtrate was concentrated. The residue was subjected to silica gel column chromatography (hexane-ethyl acetate 85:15, v/v) to give the title compound (214 mg, yield 19%) as a pale-yellow oil.

¹H-NMR (300 MHz, CDCl₃) δ:0.00 (s, 9H), 0.91-0.98 (m, 2H), 2.03 (s, 3H), 2.70 (s, 3H), 3.55-3.61 (m, 2H), 5.22-5.31 (m, 2H), 7.32-7.51 (m, 4H), 7.65 (d, J=8.3 Hz, 1H), 7.80-7.88 (m, 2H), 9.40 (s, 1H).

### Reference Example 276

### ethyl (2E)-3-(2,5-dimethyl-4-(1-naphthyl)-1-{[2-(trimethylsilyl)ethoxy]methyl}-1H-pyrrol-3-yl)acrylate

By a method similar to that in Reference Example 12, the title compound was obtained from 2,5-dimethyl-4-(1-naphthyl)-1-{[2-(trimethylsilyl)ethoxy]methyl}-1H-pyrrole-3-carbaldehyde obtained in Reference Example 275 and ethyl (diethoxyphosphoryl)acetate.

¹H-NMR (300 MHz, CDCl₃) δ:0.00 (s, 9H), 0.90-0.97 (m, 2H), 1.09 (t, J=7.0 Hz, 3H), 1.94 (s, 3H), 2.47 (s, 3H), 3.52-3.59 (m, 2H), 3.93-4.02 (m, 2H), 5.08 (d, J=15.9 Hz, 1H), 5.18-5.30 (m, 2H), 7.33 (t, J=7.4 Hz, 2H), 7.39-7.61 (m, 4H), 7.80-7.88 (m, 2H).

### Reference Example 277

### (2E)-3-(2,5-dimethyl-4-(1-naphthyl)-1-{[2-(trimethylsilyl)ethoxy]methyl}-1H-pyrrol-3-yl)acrylic acid

By a method similar to that in Reference Example 13, the title compound was obtained from ethyl (2E)-3-(2,5-dimethyl-4-(1-naphthyl)-1-{[2-(trimethylsilyl)ethoxy]methyl}-1H-pyrrol-3-yl)acrylate obtained in Reference Example 276.

¹H-NMR (300 MHz, DMSO-d₆) δ:0.00 (s, 9H), 0.85-0.93 (m, 2H), 1.92 (s, 3H), 2.43 (s, 3H), 3.58 (t, J=7.8 Hz, 2H), 4.86 (d, J=15.9 Hz, 1H), 5.27-5.38 (m, 2H), 7.28-7.61 (m, 6H), 7.91-8.00 (m, 2H).

### Reference Example 278

### benzyl {[(2-isopropoxyethyl)amino]sulfonyl}carbamate

By a method similar to that in Reference Example 102, the title compound was obtained from benzyl alcohol, chlorosulfonyl isocyanate and 2-isopropoxyethanamine.

¹H-NMR (300 MHz, CDCl₃) δ:1.13 (d, J=6.4 Hz, 6H), 3.24 (q, J=4.9 Hz, 2H), 3.47-3.60 (m, 3H), 5.19 (s, 2H), 5.49 (br s, 1H), 7.32-7.41 (m, 5H).

### Reference Example 279

### N-(2-isopropoxyethyl)sulfamide

By a method similar to that in Reference Example 109, the title compound was obtained from benzyl {[(2-isopropoxyethyl)amino]sulfonyl}carbamate obtained in Reference Example 278.

¹H-NMR (300 MHz, CDCl₃) δ:1.17 (d, J=6.2 Hz, 6H), 3.31 (q, J=5.3 Hz, 2H), 3.53-3.67 (m, 3H), 4.80 (br s, 3H).

### Reference Example 280

### 5-(5-cyclopropyl-1H-pyrrolo[2,3-b]pyridin-1-yl)-1,3-dimethyl-1H-pyrazole-4-carbaldehyde

To a mixture of 5-(5-bromo-1H-pyrrolo[2,3-b]pyridin-1-yl)-1,3-dimethyl-1H-pyrazole-4-carbaldehyde obtained in Reference Example 236 (1.52 g), cyclopropylboronic acid (818 mg), potassium carbonate (3.94 g) and toluene (50 mL) was stirred at room temperature for 30 min under argon atmosphere. Tris(dibenzylideneacetone)dipalladium(0) (109 mg) and 2-dicyclohexylphosphino-2′,6′-dimethoxybiphenyl (195 mg) was added to this reaction mixture at room temperature, and the reaction mixture was stirred at 100° C. for 18 hr under Argon. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 70:30, v/v) to give the title compound (1.31 g, yield 99%) as a colorless oil.

¹H-NMR (300 MHz, CDCl₃) δ:0.71-0.78 (m, 2H), 1.00-1.08 (m, 2H), 1.99-2.09 (m, 1H), 2.54 (s, 3H), 3.68 (s, 3H), 6.68 (d, J=3.8 Hz, 1H), 7.25-7.28 (m, 1H), 7.65 (d, J=2.3 Hz, 1H), 8.21 (d, J=1.9 Hz, 1H), 9.57 (s, 1H).

### Reference Example 281

### (E)-2-[5-(5-cyclopropyl-1H-pyrrolo[2,3-b]pyridin-1-yl)-1,3-dimethyl-1H-pyrazol-4-yl]ethylenesulfonamide

By a method similar to that in Reference Example 178, the title compound was obtained from 5-(5-cyclopropyl-1H-pyrrolo[2,3-b]pyridin-1-yl)-1,3-dimethyl-1H-pyrazole-4-carbaldehyde obtained in Reference Example 280 and tert-butyl {[(diphenylphosphoryl)methyl]sulfonyl}carbamate.

¹H-NMR (300 MHz, CDCl₃) δ:0.72-0.79 (m, 2H), 0.95-1.03 (m, 2H), 2.02-2.13 (m, 1H), 2.36 (s, 3H), 3.47 (s, 3H), 6.12 (d, J=15.6 Hz, 1H), 6.76-6.83 (m, 2H), 6.87 (s, 2H), 7.62 (d, J=3.8 Hz, 1H), 7.77 (d, J=2.1 Hz, 1H), 8.15 (d, J=2.1 Hz, 1H).

### Reference Example 282

### 2-[5-(5-cyclopropyl-1H-pyrrolo[2,3-b]pyridin-1-yl)-1,3-dimethyl-1H-pyrazol-4-yl]ethanesulfonamide

By a method similar to that in Reference Example 179, the title compound was obtained from (E)-2-[5-(5-cyclopropyl-1H-pyrrolo[2,3-b]pyridin-1-yl)-1,3-dimethyl-1H-pyrazol-4-yl]ethylenesulfonamide obtained in Reference Example 281.

¹H-NMR (300 MHz, CDCl₃) δ:0.71-0.78 (m, 2H), 0.95-1.02 (m, 2H), 2.01-2.12 (m, 1H), 2.20 (s, 3H), 2.56 (dd, J=7.5, 3.2 Hz, 2H), 2.85-2.94 (m, 2H), 3.39 (s, 3H), 6.69 (d, J=3.6 Hz, 1H), 6.76 (s, 2H), 7.62 (d, J=3.6 Hz, 1H), 7.73 (d, J=2.1 Hz, 1H), 8.13 (d, J=2.1 Hz, 1H).

### Reference Example 283

### 5-(5-fluoro-1H-pyrrolo[2,3-b]pyridin-1-yl)-1,3-dimethyl-1H-pyrazole-4-carbaldehyde

By a method similar to that in Reference Example 1, the title compound was obtained from 5-fluoro-1H-pyrrolo[2,3-b]pyridine and 5-chloro-1,3-dimethyl-1H-pyrazole-4-carbaldehyde.

¹H-NMR (300 MHz, CDCl₃) δ:2.55 (s, 3H), 3.68 (s, 3H), 6.76 (d, J=3.8 Hz, 1H), 7.38 (d, J=3.8 Hz, 1H), 7.72 (dd, J=8.5, 2.6 Hz, 1H), 8.23 (dd, J=2.4, 1.7 Hz, 1H), 9.60 (s, 1H).

### Reference Example 284

### ethyl (2E)-3-[5-(5-fluoro-1H-pyrrolo[2,3-b]pyridin-1-yl)-1,3-dimethyl-1H-pyrazol-4-yl]acrylate

By a method similar to that in Reference Example 12, the title compound was obtained from 5-(5-fluoro-1H-pyrrolo[2,3-b]pyridin-1-yl)-1,3-dimethyl-1H-pyrazole-4-carbaldehyde obtained in Reference Example 283 and ethyl (diethoxyphosphoryl)acetate.

¹H-NMR (300 MHz, CDCl₃) δ:1.24 (t, J=7.2 Hz, 3H), 2.46 (s, 3H), 3.58 (s, 3H), 4.14 (q, J=7.0 Hz, 2H), 5.70 (d, J=16.2 Hz, 1H), 6.75 (d, J=3.8 Hz, 1H), 7.25-7.31 (m, 2H), 7.72 (dd, J=8.5, 2.6 Hz, 1H), 8.22 (dd, J=2.5, 1.6 Hz, 1H).

### Reference Example 285

### (2E)-3-[5-(5-fluoro-1H-pyrrolo[2,3-b]pyridin-1-yl)-1,3-dimethyl-1H-pyrazol-4-yl]acrylic acid

By a method similar to that in Reference Example 13, the title compound was obtained from ethyl (2E)-3-[5-(5-fluoro-1H-pyrrolo[2,3-b]pyridin-1-yl)-1,3-dimethyl-1H-pyrazol-4-yl]acrylate obtained in Reference Example 284.

¹H-NMR (300 MHz, DMSO-d₆) δ:2.37 (s, 3H), 3.50 (s, 3H), 5.47 (d, J=16.2 Hz, 1H), 6.90 (d, J=3.6 Hz, 1H), 7.05 (d, J=16.2 Hz, 1H), 7.84 (d, J=3.6 Hz, 1H), 8.09 (dd, J=9.1, 2.7 Hz, 1H), 8.29 (dd, J=2.5, 1.6 Hz, 1H), 12.20 (br s, 1H).

### Reference Example 286

### benzyl [(pentylamino)sulfonyl]carbamate

To a solution of benzyl alcohol (3.06 g) in dichloromethane (150 mL) was added chlorosulfonyl isocyanate (2.55 mL) with stirring at 0° C., and the mixture was stirred at 0° C. for 30 min. Pyridine (8.0 mL) was added to this reaction mixture, and the mixture was stirred at 0° C. for 1 hr. 1-Pentylamine (16.0 mL) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried over anhydrous magnesium sulfate, and filtrated. The filtrate was concentrated, and the residue was crystallized from hexane-ethyl acetate to give the title compound (8.18 g, yield 96%) as colorless crystals.

¹H-NMR (300 MHz, CDCl₃) δ:0.85-0.92 (m, 3H), 1.25-1.34 (m, 4H), 1.46-1.63 (m, 2H), 2.98-3.07 (m, 2H), 5.07 (s, 1H), 5.19 (s, 2H), 7.28-7.42 (m, 5H).

### Reference Example 287

### N-pentylsulfamide

Benzyl [(pentylamino)sulfonyl]carbamate obtained in Reference Example 286 (5.83 g) was dissolved in a mixed solvent of tetrahydrofuran (50 mL) and ethanol (50 mL), 10% palladium carbon (3.11 g) was added, and the mixture was stirred under 1 atom of hydrogen atmosphere at room temperature for 4 hr. The catalyst was removed by filtration, and the filtrate was concentrated. The residue was crystallized from diisopropyl ether-ethyl acetate to give the title compound (3.15 g, yield 98%) as colorless crystals.

¹H-NMR (300 MHz, CDCl₃) δ:0.87-0.95 (m, 3H), 1.30-1.40 (m, 4H), 1.52-1.63 (m, 2H), 3.10-3.16 (m, 2H), 4.51 (br s, 3H).

### Reference Example 288

### 3-[1,3-dimethyl-5-(5-methyl-1H-pyrrolo[2,3-b]pyridin-1-yl)-1H-pyrazol-4-yl]propanoic acid

By a method similar to that in Reference Example 65, the title compound was obtained from (2E)-3-[1,3-dimethyl-5-(5-methyl-1H-pyrrolo[2,3-b]pyridin-1-yl)-1H-pyrazol-4-yl]acrylic acid obtained in Reference Example 206.

¹H-NMR (300 MHz, CDCl₃) δ:2.28 (s, 3H), 2.29-2.36 (m, 2H), 2.45 (s, 3H), 2.53-2.72 (m, 2H), 3.45 (s, 3H), 6.63 (d, J=3.6 Hz, 1H), 7.15 (d, J=3.6 Hz, 1H), 7.82 (d, J=1.1 Hz, 1H), 8.14 (d, J=2.1 Hz, 1H).

### Reference Example 289

### 3-[1,3-dimethyl-5-(5-methyl-1H-pyrrolo[2,3-b]pyridin-1-yl)-1H-pyrazol-4-yl]propan-1-ol

To a solution of 3-[1,3-dimethyl-5-(5-methyl-1H-pyrrolo[2,3-b]pyridin-1-yl)-1H-pyrazol-4-yl]propanoic acid obtained in Reference Example 288 (410 mg) and N,N-dimethylformamide (0.1 mL) in tetrahydrofuran (13 mL) was added dropwise oxalyl chloride (261.8 mg), and the mixture was stirred at room temperature for 1 hr. The reaction mixture was concentrated under reduced pressure, tetrahydrofuran (13 mL) and methanol (13 mL) were added to the residue. The mixture was stirred at room temperature for 2 hr. Water was added to the reaction mixture, and the mixture was extracted with ethyl acetate. The organic layer was washed with saturated brine, dried over anhydrous magnesium sulfate, and filtrated. The filtrate was concentrated. Tetrahydrofuran (13 mL) was added to the residue, which was cooled at 0° C. in an ice bath, was added diisobutylaluminum hydride (1.5M toluene solution, 13 mL) by small portions with stirring. The reaction mixture was stirred at room temperature for 2 hr, and was cooled again at 0° C. in an ice bath. Methanol and water were added to the reaction mixture with stirring, the mixture was filtrated through Celite®, and the filtrate was concentrated. The residue was subjected to silica gel column chromatography (ethyl acetate-methanol 95:5, v/v) to give the title compound (291 mg, yield 74%) as a colorless oil.

¹H-NMR (300 MHz, CDCl₃) δ:1.54-1.72 (m, 2H), 2.29 (s, 3H), 2.33-2.40 (m, 2H), 2.44 (s, 3H), 3.42-3.53 (m, 5H), 6.61 (d, J=3.6 Hz, 1H), 7.14 (d, J=3.6 Hz, 1H), 7.79 (d, J=1.3 Hz, 1H), 8.16 (d, J=2.1 Hz, 1H).

### Example 1

### (2E)-3-[5-(1H-indol-1-yl)-1,3-dimethyl-1H-pyrazol-4-yl]-N-(pentylsulfonyl)acrylamide

A mixture of (2E)-3-[5-(1H-indol-1-yl)-1,3-dimethyl-1H-pyrazol-4-yl]acrylic acid obtained in Reference Example 2 (400 mg), 2-methyl-6-nitrobenzoic anhydride (586 mg), pentane-1-sulfonamide (240 mg), triethylamine (465 mg), 4-dimethylaminopyridine (175 mg) and acetonitrile (8 mL) was stirred at room temperature for 24 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 40:60, v/v) to give the title compound (579 mg, yield 98%) as a colorless amorphous solid.

¹H-NMR (300 MHz, CDCl₃) δ:0.87 (t, J=7.1 Hz, 3H), 1.25-1.40 (m, 4H), 1.72-1.76 (m, 2H), 2.44 (s, 3H), 3.28-3.34 (m, 2H), 3.54 (s, 3H), 5.23 (d, J=15.6 Hz, 1H), 6.83 (d, J=3.2 Hz, 1H), 6.94-7.01 (m, 1H), 7.07 (d, J=3.2 Hz, 1H), 7.23-7.27 (m, 2H), 7.40 (s, 1H), 7.48 (d, J=15.6 Hz, 1H), 7.73-7.75 (m, 1H).

### Example 2

### 3-[5-(1H-indol-1-yl)-1,3-dimethyl-1H-pyrazol-4-yl]-N-(pentylsulfonyl)propanamide

(2E)-3-[5-(1H-Indol-1-yl)-1,3-dimethyl-1H-pyrazol-4-yl]-N-(pentylsulfonyl)acrylamide obtained in Example 1 (233 mg) was dissolved in a mixed solvent of tetrahydrofuran (10 mL) and methanol (10 mL), 10% palladium carbon (25 mg) was added, and the mixture was stirred under 1 atom of hydrogen atmosphere at room temperature for 6 hr. The catalyst was removed by filtration, and the filtrate was concentrated. The residue was subjected to silica gel column chromatography (hexane-ethyl acetate 55:45, v/v), and crystallized from hexane-ethyl acetate to give the title compound (174 mg, yield 74%) as colorless crystals.

¹H-NMR (300 MHz, CDCl₃) δ:0.86-0.92 (m, 3H), 1.25-1.40 (m, 4 H), 1.65-1.75 (m, 2H), 2.01-2.15 (m, 2H), 2.30 (s, 3H), 2.62 (t, J=7.6 Hz, 2H), 3.23-3.30 (m, 2H), 3.47 (s, 3H), 6.76 (d, J=3.4 Hz, 1H), 6.96-7.02 (m, 1H), 7.09 (d, J=3.4 Hz, 1H), 7.20-7.27 (m, 2H), 7.39 (s, 1H), 7.70-7.73 (m, 1H).

### Example 3

### (2E)-3-[5-(1H-indazol-1-yl)-1,3-dimethyl-1H-pyrazol-4-yl]-N-(pentylsulfonyl)acrylamide

By a method similar to that in Example 1, the title compound was obtained from (2E)-3-[5-(1H-indazol-1-yl)-1,3-dimethyl-1H-pyrazol-4-yl]acrylic acid obtained in Reference Example 5 and pentane-1-sulfonamide.

¹H-NMR (300 MHz, CDCl₃) δ:0.88 (t, J=7.0 Hz, 3H), 1.22-1.43 (m, 4H), 1.66-1.78 (m, 2H), 2.46 (s, 3H), 3.31-3.36 (m, 2H), 3.57 (s, 3H), 5.45 (d, J=15.9 Hz, 1H), 7.14 (d, J=8.3 Hz, 1H), 7.29-7.33 (m, 1H), 7.44-7.51 (m, 2H), 7.64 (s, 1 H), 7.88 (d, J=8.0 Hz, 1H), 8.32 (d, J=1.5 Hz, 1H).

### Example 4

### (2E)-3-[5-(2H-indazol-2-yl)-1,3-dimethyl-1H-pyrazol-4-yl]-N-(pentylsulfonyl)acrylamide

By a method similar to that in Example 1, the title compound was obtained from (2E)-3-[5-(2H-indazol-2-yl)-1,3-dimethyl-1H-pyrazol-4-yl]acrylic acid obtained in Reference Example 6 and pentane-1-sulfonamide.

¹H-NMR (300 MHz, CDCl₃) δ:0.87 (t, J=7.0 Hz, 3H), 1.23-1.42 (m, 4H), 1.68-1.80 (m, 2H), 2.44 (s, 3H), 3.30-3.37 (m, 2H), 3.69 (s, 3H), 5.70 (d, J=15.9 Hz, 1H), 7.18-7.24 (m, 1 H), 7.42-7.45 (m, 1H), 7.49 (d, J=15.9 Hz, 1H), 7.73-7.78 (m, 2H), 7.90 (s, 1H), 8.13 (s, 1H).

### Example 5

### (2E)-3-[5-(1H-benzimidazol-1-yl)-1,3-dimethyl-1H-pyrazol-4-yl]-N-(pentylsulfonyl)acrylamide

By a method similar to that in Example 1, the title compound was obtained from (2E)-3-[5-(1H-benzimidazol-1-yl)-1,3-dimethyl-1H-pyrazol-4-yl]acrylic acid obtained in Reference Example 8 and pentane-1-sulfonamide.

¹H-NMR (300 MHz, CDCl₃) δ:0.81 (t, J=7.1 Hz, 3H), 1.18-1.36 (m, 4H), 1.58-1.67 (m, 2H), 2.49 (s, 3H), 3.30-3.36 (m, 2 H), 3.55 (s, 3H), 5.67 (d, J=16.0 Hz, 1H), 7.12 (d, J=7.5 Hz, 1H), 7.26-7.40 (m, 2H), 7.50 (d, J=8.1 Hz, 1H), 7.65 (d, J=16.0 Hz, 1H), 7.76 (s, 1H), 10.30 (s, 1H).

### Example 6

### (2E)-3-[5-(1-benzothien-3-yl)-1,3-dimethyl-1H-pyrazol-4-yl]-N-(pentylsulfonyl)acrylamide

By a method similar to that in Example 1, the title compound was obtained from (2E)-3-[5-(1-benzothien-3-yl)-1,3-dimethyl-1H-pyrazol-4-yl]acrylic acid obtained in Reference Example 10 and pentane-1-sulfonamide.

¹H-NMR (300 MHz, CDCl₃) δ:0.84-0.90 (m, 3H), 1.23-1.40 (m, 4 H), 1.68-1.79 (m, 2H), 2.47 (s, 3H), 3.31-3.37 (m, 2H), 3.64 (s, 3H), 5.75 (d, J=15.8 Hz, 1H), 7.35-7.50 (m, 3H), 7.50-7.58 (m, 2H), 7.68 (s, 1H), 7.96-7.99 (m, 1H).

### Example 7

### potassium {(2E)-3-[5-(1H-indol-1-yl)-1,3-dimethyl-1H-pyrazol-4-yl]prop-2-enoyl}(pentylsulfonyl)azanide

To a solution of (2E)-3-[5-(1H-indol-1-yl)-1,3-dimethyl-1H-pyrazol-4-yl]-N-(pentylsulfonyl)acrylamide obtained in Example 1 (445 mg) in methanol (4 mL) was added an aqueous solution (1 mL) of potassium hydrogencarbonate (108 mg), and the mixture was stirred at room temperature for 1 hr. The reaction mixture was concentrated under reduced pressure, and the residue was crystallized from diethyl ether-methanol to give the title compound (483 mg, yield 99%) as colorless crystals.

¹H-NMR (300 MHz, DMSO-d₆) δ:0.78-0.82 (m, 3H), 1.15-1.23 (m, 4H), 1.37-1.47 (m, 2H), 2.34 (s, 3H), 2.82-2.87 (m, 2H), 3.41 (s, 3H), 5.57 (d, J=16.1 Hz, 1H), 6.76 (d, J=16.1 Hz, 1H), 6.81 (d, J=3.4 Hz, 1H), 6.95-6.99 (m, 1H), 7.14-7.22 (m, 2H), 7.51 (d, J=3.4 Hz, 1H), 7.68-7.71 (m, 1H).

### Example 8

### potassium {(2E)-3-[5-(1H-indazol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(1H-indazol-1-yl)-1,3-dimethyl-1H-pyrazol-4-yl]-N-(pentylsulfonyl)acrylamide obtained in Example 3.

¹H-NMR (300 MHz, DMSO-d₆) δ:0.76-0.84 (m, 3H), 1.15-1.24 (m, 4H), 1.35-1.47 (m, 2H), 2.36 (s, 3H), 2.81-2.87 (m, 2H), 3.44 (s, 3H), 5.58 (d, J=16.2 Hz, 1H), 6.76 (d, J=16.2 Hz, 1H), 7.22 (dd, J=8.5, 0.9 Hz, 1H), 7.29-7.35 (m, 1H), 7.45-7.50 (m, 1H), 7.95 (d, J=8.1 Hz, 1H), 8.55 (d, J=0.9 Hz, 1H).

### Example 9

### (2E)-3-[1,3-dimethyl-5-(1H-pyrrolo[2,3-b]pyridin-1-yl)-1H-pyrazol-4-yl]-N-(pentylsulfonyl)acrylamide

A mixture of (2E)-3-[1,3-dimethyl-5-(1H-pyrrolo[2,3-b]pyridin-1-yl)-1H-pyrazol-4-yl]acrylic acid obtained in Reference Example 13 (473 mg), 2-methyl-6-nitrobenzoic anhydride (689 mg), pentane-1-sulfonamide (269 mg), triethylamine (525 mg), 4-dimethylaminopyridine (206 mg) and acetonitrile (8 mL) was stirred at room temperature for 72 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and the residue was crystallized from hexane-ethanol to give the title compound (590 mg, yield 85%) as colorless crystals.

¹H-NMR (300 MHz, CDCl₃) δ:0.85-0.92 (m, 3H), 1.23-1.44 (m, 4H), 1.71-1.83 (m, 2H), 2.29 (s, 3H), 3.37-3.43 (m, 2H), 3.56 (s, 3H), 5.57 (d, J=15.7 Hz, 1H), 6.78 (d, J=3.4 Hz, 1H), 7.18 (d, J=3.4 Hz, 1H), 7.23 (dd, J=7.8, 4.7 Hz, 1H), 7.34 (d, J=15.7 Hz, 1H), 8.05 (dd, J=7.8, 1.5 Hz, 1H), 8.32 (dd, J=4.7, 1.5 Hz, 1H), 8.88 (s, 1H).

Recrystallization of the crude crystals obtained under the same conditions as in Example 9 from hexane-diisopropyl ether-ethanol gave colorless crystals. melting point 149-163° C.

Recrystallization of the crude crystals obtained under the same conditions as in Example 9 from H₂O-95% ethanol (ethanol-H₂O 95:5, v/v) gave colorless crystals. melting point 194-197° C.

### Example 10

### (2E)-3-[1,3-dimethyl-5-(1-naphthyl)-1H-pyrazol-4-yl]-N-(pentylsulfonyl)acrylamide

By a method similar to that in Example 1, the title compound was obtained from (2E)-3-[1,3-dimethyl-5-(1-naphthyl)-1H-pyrazol-4-yl]acrylic acid obtained in Reference Example 15 and pentane-1-sulfonamide.

¹H-NMR (300 MHz, CDCl₃) δ:0.86 (t, J=7.2 Hz, 3H), 1.23-1.34 (m, 4H), 1.67-1.75 (m, 2H), 2.49 (s, 3H), 3.26-3.31 (m, 2H), 3.51 (s, 3H), 5.60 (d, J=15.5 Hz, 1H), 7.31-7.65 (m, 7H), 7.97 (d, J=8.0 Hz, 1H), 8.04 (d, J=8.3 Hz, 1H).

### Example 11

### potassium {(2E)-3-[1,3-dimethyl-5-(1-naphthyl)-1H-pyrazol-4-yl]prop-2-enoyl}(pentylsulfonyl)azanide

By a method similar to that in Example 7, the title compound was obtained from (2E)-3-[1,3-dimethyl-5-(1-naphthyl)-1H-pyrazol-4-yl]-N-(pentylsulfonyl)acrylamide obtained in Example 10.

¹H-NMR (300 MHz, DMSO-d₆) δ:0.75-0.82 (m, 3H), 1.12-1.23 (m, 4H), 1.35-1.48 (m, 2H), 2.37 (s, 3H), 2.78-2.85 (m, 2H), 3.40 (s, 3H), 5.70 (d, J=16.1 Hz, 1H), 6.77 (d, J=16.1 Hz, 1H), 7.32 (d, J=8.0 Hz, 1H), 7.48-7.63 (m, 3H), 7.64-7.73 (m, 1H), 8.07 (d, J=8.0 Hz, 1H), 8.12 (d, J=8.0 Hz, 1H).

### Example 12

### (2E)-3-[1,3-dimethyl-5-(4-methyl-1H-indol-1-yl)-1H-pyrazol-4-yl]-N-(pentylsulfonyl)acrylamide

By a method similar to that in Example 1, the title compound was obtained from (2E)-3-[1,3-dimethyl-5-(4-methyl-1H-indol-1-yl)-1H-pyrazol-4-yl]acrylic acid obtained in Reference Example 17 and pentane-1-sulfonamide.

¹H-NMR (300 MHz, DMSO-d₆) δ:0.81 (t, J=7.2 Hz, 3H), 1.15-1.36 (m, 4H), 1.52-1.62 (m, 2H), 2.40 (s, 3H), 2.56 (s, 3H), 3.28-3.33 (m, 2H), 3.46 (s, 3H), 6.12 (d, J=15.9 Hz, 1H), 6.82 (d, J=8.0 Hz, 1H), 6.89 (d, J=3.4 Hz, 1H), 6.97-7.15 (m, 3H), 7.55 (d, J=3.4 Hz, 1H), 11.59 (s, 1H).

### Example 13

### potassium {(2E)-3-[1,3-dimethyl-5-(4-methyl-1H-indol-1-yl)-1H-pyrazol-4-yl]prop-2-enoyl}(pentylsulfonyl)azanide

By a method similar to that in Example 7, the title compound was obtained from (2E)-3-[1,3-dimethyl-5-(4-methyl-1H-indol-1-yl)-1H-pyrazol-4-yl]-N-(pentylsulfonyl)acrylamide obtained in Example 12.

¹H-NMR (300 MHz, DMSO-d₆) δ:0.78-0.82 (m, 3H), 1.15-1.25 (m, 4H), 1.38-1.52 (m, 2H), 2.33 (s, 3H), 2.55 (s, 3H), 2.82-2.87 (m, 2H), 3.39 (s, 3H), 5.58 (d, J=16.2 Hz, 1H), 6.74-6.85 (m, 3H), 6.97 (d, J=7.2 Hz, 1H), 7.06-7.12 (m, 1H), 7.48 (d, J=3.2 Hz, 1H).

### Example 14

### (2E)-3-[5-(4-chloro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(4-chloro-1H-indol-1-yl)-1,3-dimethyl-1H-pyrazol-4-yl]acrylic acid obtained in Reference Example 19 and pentane-1-sulfonamide.

¹H-NMR (300 MHz, CDCl₃) δ:0.84-0.92 (m, 3H), 1.23-1.40 (m, 4H), 1.68-1.80 (m, 2H), 2.44 (s, 3H), 3.29-3.34 (m, 2H), 3.52 (s, 3H), 5.35 (d, J=15.5 Hz, 1H), 6.89 (d, J=8.0 Hz, 1H), 6.95 (d, J=3.4 Hz, 1H), 7.13 (d, J=3.4 Hz, 1H), 7.16-7.21 (m, 1H), 7.25-7.29 (m, 1H), 7.46 (d, J=15.5 Hz, 1H), 7.74 (s, 1H).

### Example 15

### potassium {(2E)-3-[5-(4-chloro-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(4-chloro-1H-indol-1-yl)-1,3-dimethyl-1H-pyrazol-4-yl]-N-(pentylsulfonyl)acrylamide obtained in Example 14.

¹H-NMR (300 MHz, DMSO-d₆) δ:0.76-0.84 (m, 3H), 1.16-1.25 (m, 4H), 1.38-1.47 (m, 2H), 2.34 (s, 3H), 2.82-2.87 (m, 2H), 3.42 (s, 3H), 5.54 (d, J=16.2 Hz, 1H), 6.74 (d, J=16.2 Hz, 1H), 6.85 (dd, J=3.4, 0.9 Hz, 1H), 6.99 (d, J=7.9 Hz, 1H), 7.16-7.24 (m, 1H), 7.24-7.29 (m, 1H), 7.68 (d, J=3.4 Hz, 1H).

### Example 16

### (2E)-3-[5-(5-fluoro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(5-fluoro-1H-indol-1-yl)-1,3-dimethyl-1H-pyrazol-4-yl]acrylic acid obtained in Reference Example 21 and pentane-1-sulfonamide.

¹H-NMR (300 MHz, DMSO-d₆) δ:0.78-0.84 (m, 3H), 1.18-1.35 (m, 4H), 1.51-1.63 (m, 2H), 2.39 (s, 3H), 3.27-3.37 (m, 2H), 3.47 (s, 3H), 6.06 (d, J=16.0 Hz, 1H), 6.86 (d, J=3.4 Hz, 1H), 7.00-7.11 (m, 3H), 7.49-7.54 (m, 1H), 7.66 (d, J=3.4 Hz, 1H), 11.60 (s, 1H).

### Example 17

### potassium {(2E)-3-[5-(5-fluoro-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(5-fluoro-1H-indol-1-yl)-1,3-dimethyl-1H-pyrazol-4-yl]-N-(pentylsulfonyl)acrylamide obtained in Example 16.

¹H-NMR (300 MHz, DMSO-d₆) δ:0.78-0.83 (m, 3H), 1.17-1.25 (m, 4H), 1.37-1.48 (m, 2H), 2.34 (s, 3H), 2.82-2.87 (m, 2H), 3.41 (s, 3H), 5.56 (d, J=16.2 Hz, 1H), 6.76 (d, J=16.2 Hz, 1H), 6.81 (d, J=3.4 Hz, 1H), 6.95-7.07 (m, 2H), 7.48 (dd, J=9.6, 1.9 Hz, 1H), 7.61 (d, J=3.4 Hz, 1H).

### Example 18

### (2E)-3-[5-(5-methoxy-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(5-methoxy-1H-indol-1-yl)-1,3-dimethyl-1H-pyrazol-4-yl]acrylic acid obtained in Reference Example 23 and pentane-1-sulfonamide.

¹H-NMR (300 MHz, CDCl₃) δ:0.85-0.89 (m, 3H), 1.21-1.38 (m, 4H), 1.65-1.78 (m, 2H), 2.42 (s, 3H), 3.30-3.35 (m, 2H), 3.53 (s, 3H), 3.88 (s, 3H), 5.24 (d, J=15.7 Hz, 1H), 6.75 (d, J=3.4 Hz, 1H), 6.84-6.93 (m, 2H), 7.04 (d, J=3.4 Hz, 1H), 7.17 (s, 1H), 7.48 (d, J=15.7 Hz, 1H), 7.68 (s, 1H).

### Example 19

### potassium {(2E)-3-[5-(5-methoxy-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(5-methoxy-1H-indol-1-yl)-1,3-dimethyl-1H-pyrazol-4-yl]-N-(pentylsulfonyl)acrylamide obtained in Example 18.

¹H-NMR (300 MHz, DMSO-d₆) δ:0.78-0.83 (m, 3H), 1.18-1.22 (m, 4H), 1.40-1.48 (m, 2H), 2.33 (s, 3H), 2.84-2.89 (m, 2H), 3.41 (s, 3H), 3.79 (s, 3H), 5.58 (d, J=16.3 Hz, 1H), 6.69-6.89 (m, 4H), 7.20 (d, J=2.3 Hz, 1H), 7.45 (d, J=3.4 Hz, 1H).

### Example 20

### (2E)-3-[5-(6-chloro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(6-chloro-1H-indol-1-yl)-1,3-dimethyl-1H-pyrazol-4-yl]acrylic acid obtained in Reference Example 25 and pentane-1-sulfonamide.

¹H-NMR (300 MHz, CDCl₃) δ:0.85-0.91 (m, 3H), 1.24-1.39 (m, 4H), 1.68-1.78 (m, 2H), 2.44 (s, 3H), 3.29-3.34 (m, 2H), 3.53 (s, 3H), 5.33 (d, J=15.9 Hz, 1H), 6.81 (d, J=3.0 Hz, 1H), 6.97 (d, J=1.7 Hz, 1H), 7.06 (d, J=3.0 Hz, 1H), 7.23 (dd, J=8.4, 1.7 Hz, 1H), 7.45 (d, J=15.9 Hz, 1H), 7.47 (s, 1H), 7.65 (d, J=8.4 Hz, 1H).

### Example 21

### (2E)-3-{5-[6-(benzyloxy)-1H-indol-1-yl]-1,3-dimethyl-1H-pyrazol-4-yl}-N-(pentylsulfonyl)acrylamide

By a method similar to that in Example 1, the title compound was obtained from (2E)-3-{5-[6-(benzyloxy)-1H-indol-1-yl]-1,3-dimethyl-1H-pyrazol-4-yl}acrylic acid obtained in Reference Example 28 and pentane-1-sulfonamide.

¹H-NMR (300 MHz, CDCl₃) δ:0.84-0.89 (m, 3H), 1.27-1.38 (m, 4H), 1.67-1.80 (m, 2H), 2.44 (s, 3H), 3.30-3.35 (m, 2H), 3.47 (s, 3H), 5.01 (s, 2H), 5.25 (d, J=15.6 Hz, 1H), 6.47 (d, J=2.3 Hz, 1H), 6.72-6.75 (m, 1H), 6.94 (d, J=3.2 Hz, 1H), 6.99 (dd, J=8.7, 2.3 Hz, 1H), 7.29-7.51 (m, 7H), 7.60 (d, J=8.7 Hz, 1H).

### Example 22

### (2E)-3-[5-(6-hydroxy-1H-indol-1-yl)-1,3-dimethyl-1H-pyrazol-4-yl]-N-(pentylsulfonyl)acrylamide

To a solution of (2E)-3-{5-[6-(benzyloxy)-1H-indol-1-yl]-1,3-dimethyl-1H-pyrazol-4-yl}-N-(pentylsulfonyl)acrylamide obtained in Example 21 (300 mg) in dichloromethane (4 mL) was added dropwise boron tribromide (1M dichloromethane solution, 1.2 mL) with stirring at −78° C., and the mixture was stirred at −78° C. for 20 min. The reaction mixture was quenched with methanol (2 mL), and concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v) to give the title compound (233 mg, yield 93%) as a colorless amorphous solid.

¹H-NMR (300 MHz, CDCl₃) δ:0.88 (t, J=7.0 Hz, 3H), 1.24-1.43 (m, 4H), 1.70-1.81 (m, 2H), 2.35 (s, 3H), 3.32-3.40 (m, 2H), 3.51 (s, 3H), 5.36-5.43 (m, 1H), 6.15-6.31 (m, 2H), 6.73 (d, J=3.6 Hz, 1H), 6.83 (dd, J=8.5, 2.1 Hz, 1H), 6.92-6.93 (m, 1H), 7.43 (d, J=15.5 Hz, 1H), 7.55 (d, J=8.5 Hz, 1H), 8.02 (s, 1H).

### Example 23

### (2E)-3-[1,3-dimethyl-5-(2-naphthyl)-1H-pyrazol-4-yl]-N-(pentylsulfonyl)acrylamide

By a method similar to that in Example 1, the title compound was obtained from (2E)-3-[1,3-dimethyl-5-(2-naphthyl)-1H-pyrazol-4-yl]acrylic acid obtained in Reference Example 30 and pentane-1-sulfonamide.

¹H-NMR (300 MHz, CDCl₃) δ:0.87 (t, J=7.0 Hz, 3H), 1.25-1.42 (m, 4H), 1.69-1.83 (m, 2H), 2.45 (s, 3H), 3.32-3.41 (m, 2H), 3.72 (s, 3H), 5.87 (d, J=15.5 Hz, 1H), 7.37 (dd, J=8.3, 1.9 Hz, 1H), 7.56-7.67 (m, 4H), 7.79 (s, 1H), 7.87-7.96 (m, 2H), 8.00 (d, J=8.7 Hz, 1H).

### Example 24

### (2E)-3-[1,3-dimethyl-5-(quinolin-8-yl)-1H-pyrazol-4-yl]-N-(pentylsulfonyl)acrylamide

By a method similar to that in Example 1, the title compound was obtained from (2E)-3-[1,3-dimethyl-5-(quinolin-8-yl)-1H-pyrazol-4-yl]acrylic acid obtained in Reference Example 33 and pentane-1-sulfonamide.

¹H-NMR (300 MHz, CDCl₃) δ:0.87 (t, J=7.1 Hz, 3H), 1.23-1.44 (m, 4H), 1.70-1.85 (m, 2H), 2.28 (s, 3H), 3.43-3.49 (m, 2H), 3.53 (s, 3H), 5.72 (d, J=15.6 Hz, 1H), 7.39 (d, J=15.6 Hz, 1H), 7.52 (dd, J=8.3, 4.1 Hz, 1H), 7.62-7.76 (m, 2H), 8.03 (dd, J=7.6, 2.0 Hz, 1H), 8.31 (dd, J=8.3, 1.6 Hz, 1H), 8.78-8.87 (m, 1H), 8.92 (dd, J=4.1, 1.6 Hz, 1H).

### Example 25

### (2E)-3-[5-(5,6-difluoro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(5,6-difluoro-1H-indol-1-yl)-1,3-dimethyl-1H-pyrazol-4-yl]acrylic acid obtained in Reference Example 36 and pentane-1-sulfonamide.

¹H-NMR (300 MHz, CDCl₃) δ:0.85-0.91 (m, 3H), 1.27-1.40 (m, 4H), 1.69-1.79 (m, 2H), 2.44 (s, 3H), 3.32-3.39 (m, 2H), 3.53 (s, 3H), 5.36 (d, J=15.9 Hz, 1H), 6.73-6.81 (m, 2H), 7.10 (d, J=3.4 Hz, 1H), 7.38-7.58 (m, 3H).

### Example 26

### potassium {(2E)-3-[5-(5,6-difluoro-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(5,6-difluoro-1H-indol-1-yl)-1,3-dimethyl-1H-pyrazol-4-yl]-N-(pentylsulfonyl)acrylamide obtained in Example 25.

¹H-NMR (300 MHz, DMSO-d₆) δ:0.77-0.83 (m, 3H), 1.14-1.25 (m, 4H), 1.38-1.50 (m, 2H), 2.33 (s, 3H), 2.82-2.90 (m, 2H), 3.43 (s, 3H), 5.52 (d, J=16.3 Hz, 1H), 6.75 (d, J=16.3 Hz, 1H), 6.82 (d, J=3.0 Hz, 1H), 7.09 (dd, J=10.8, 7.0 Hz, 1H), 7.61 (d, J=3.0 Hz, 1H), 7.72 (dd, J=10.8, 8.0 Hz, 1H).

### Example 27

### (2E)-3-[5-(5-chloro-1H-indol-1-yl)-1,3-dimethyl-1H-pyrazol-4-yl]-N-(pentylsulfonyl)acrylamide

A mixture of 4-dimethylaminopyridine (643 mg), N-[3-(dimethylamino)propyl]-N′-ethylcarbodiimide hydrochloride (875 mg) and acetonitrile (10 mL) was stirred at room temperature for 10 min. Pentane-1-sulfonamide (541 mg) and (2E)-3-[5-(5-chloro-1H-indol-1-yl)-1,3-dimethyl-1H-pyrazol-4-yl]acrylic acid obtained in Reference Example 38 (1.11 g) were successively added to the reaction mixture, and the mixture was stirred at room temperature for 15 hr. 1M Hydrochloric acid was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50-40:60, v/v) to give the title compound (927 mg, yield 59%) as a colorless amorphous solid.

¹H-NMR (300 MHz, CDCl₃) δ:0.83-0.93 (m, 3H), 1.27-1.41 (m, 4H), 1.67-1.79 (m, 2H), 2.44 (s, 3H), 3.27-3.36 (m, 2H), 3.53 (s, 3H), 5.30 (d, J=15.8 Hz, 1H), 6.78 (dd, J=3.3, 0.8 Hz, 1H), 6.91 (d, J=8.7 Hz, 1H), 7.10 (d, J=3.2 Hz, 1H), 7.22 (dd, J=8.7, 2.1 Hz, 1H), 7.39 (br s, 1H), 7.46 (d, J=15.6 Hz, 1H), 7.71 (d, J=1.7 Hz, 1H).

### Example 28

### sodium {(2E)-3-[5-(5-chloro-1H-indol-1-yl)-1,3-dimethyl-1H-pyrazol-4-yl]prop-2-enoyl}(pentylsulfonyl)azanide

To a solution of (2E)-3-[5-(5-chloro-1H-indol-1-yl)-1,3-dimethyl-1H-pyrazol-4-yl]-N-(pentylsulfonyl)acrylamide obtained in Example 27 (173 mg) in methanol (1 mL) was added an aqueous solution (0.5 mL) of sodium hydrogencarbonate (35 mg), and the mixture was stirred at room temperature for 6 hr. The reaction mixture was concentrated under reduced pressure, and the residue was crystallized from diethyl ether-methanol to give the title compound (174 mg, yield 95%) as colorless crystals.

¹H-NMR (300 MHz, DMSO-d₆) δ:0.78-0.83 (m, 3H), 1.15-1.23 (m, 4H), 1.39-1.49 (m, 2H), 2.34 (s, 3H), 2.82-2.87 (m, 2H), 3.41 (s, 3H), 5.56 (d, J=16.1 Hz, 1H), 6.76 (d, J=16.1 Hz, 1H), 6.81 (dd, J=3.2, 0.8 Hz, 1H), 7.01 (d, J=8.7 Hz, 1H), 7.19 (dd, J=8.7, 1.9 Hz, 1H), 7.62 (d, J=3.2 Hz, 1H), 7.77 (d, J=1.9 Hz, 1H).

### Example 29

### (2E)-3-[5-(3-chloro-1H-indazol-1-yl)-1,3-dimethyl-1H-pyrazol-4-yl]-N-(pentylsulfonyl)acrylamide

By a method similar to that in Example 1, the title compound was obtained from (2E)-3-[5-(3-chloro-1H-indazol-1-yl)-1,3-dimethyl-1H-pyrazol-4-yl]acrylic acid obtained in Reference Example 40 and pentane-1-sulfonamide.

¹H-NMR (300 MHz, CDCl₃) δ:0.87 (t, J=7.2 Hz, 3H), 1.27-1.37 (m, 4H), 1.68-1.75 (m, 2H), 2.44 (s, 3H), 3.31-3.35 (m, 2H), 3.58 (s, 3H), 5.64 (d, J=15.6 Hz, 1H), 7.11 (d, J=8.1 Hz, 1H), 7.36-7.45 (m, 2H), 7.50-7.56 (m, 1H), 7.81 (d, J=8.1 Hz, 1H), 8.19 (br s, 1H).

### Example 30

### (2E)-3-{1,3-dimethyl-5-[6-(trifluoromethyl)-1H-indol-1-yl]-1H-pyrazol-4-yl}-N-(pentylsulfonyl)acrylamide

By a method similar to that in Example 1, the title compound was obtained from (2E)-3-{1,3-dimethyl-5-[6-(trifluoromethyl)-1H-indol-1-yl]-1H-pyrazol-4-yl}acrylic acid obtained in Reference Example 42 and pentane-1-sulfonamide.

¹H-NMR (300 MHz, DMSO-d₆) δ:0.66-0.90 (m, 3H), 1.17-1.38 (m, 4H), 1.42-1.68 (m, 2H), 2.41 (s, 3H), 3.25-3.30 (m, 2H), 3.49 (s, 3H), 5.82-6.15 (m, 1H), 6.93-7.14 (m, 2H), 7.29-7.40 (m, 1H), 7.52 (dd, J=8.7, 1.5 Hz, 1H), 7.85 (d, J=3.4 Hz, 1H), 7.96 (d, J=8.3 Hz, 1H), 11.58 (s, 1H).

### Example 31

### potassium ((2E)-3-{1,3-dimethyl-5-[6-(trifluoromethyl)-1H-indol-1-yl]-1H-pyrazol-4-yl}prop-2-enoyl)(pentylsulfonyl)azanide

By a method similar to that in Example 7, the title compound was obtained from (2E)-3-{1,3-dimethyl-5-[6-(trifluoromethyl)-1H-indol-1-yl]-1H-pyrazol-4-yl}-N-(pentylsulfonyl)acrylamide obtained in Example 30.

¹H-NMR (300 MHz, DMSO-d₆) δ:0.61-0.95 (m, 3H), 1.19-1.29 (m, 4H), 1.32-1.51 (m, 2H), 2.36 (s, 3H), 2.69-2.98 (m, 2H), 3.22-3.33 (m, 2H), 3.43 (s, 1H), 5.51 (d, J=15.9 Hz, 1H), 6.75 (d, J=16.3 Hz, 1H), 6.98 (d, J=3.4 Hz, 1H), 7.28 (s, 1H), 7.49 (d, J=8.3 Hz, 1H), 7.81 (d, J=3.0 Hz, 1H), 7.94 (d, J=8.3 Hz, 1H).

### Example 32

### 3-[1,3-dimethyl-5-(1H-pyrrolo[2,3-b]pyridin-1-yl)-1H-pyrazol-4-yl]-N-(pentylsulfonyl)propanamide

By a method similar to that in Example 1, the title compound was obtained from 3-[1,3-dimethyl-5-(1H-pyrrolo[2,3-b]pyridin-1-yl)-1H-pyrazol-4-yl]propanoic acid obtained in Reference Example 45 and pentane-1-sulfonamide.

¹H-NMR (300 MHz, CDCl₃) δ:0.88-0.95 (m, 3H), 1.32-1.45 (m, 4H), 1.50-1.62 (m, 1H), 1.64-1.80 (m, 1H), 1.99-2.13 (m, 1H), 2.26 (s, 3H), 2.31-2.38 (m, 1H), 2.54-2.66 (m, 1H), 2.86-3.00 (m, 1H), 3.03-3.16 (m, 1H), 3.23-3.37 (m, 4H), 6.78 (d, J=3.8 Hz, 1H), 7.20 (d, J=3.8 Hz, 1H), 7.29 (dd, J=7.9, 4.9 Hz, 1H), 8.11 (dd, J=7.9, 1.4 Hz, 1H), 8.43 (dd, J=4.9, 1.4 Hz, 1H), 12.12 (s, 1H).

### Example 33

### (2E)-3-[1,3-dimethyl-5-(1H-pyrrolo[2,3-b]pyridin-1-yl)-1H-pyrazol-4-yl]-N-[(4-methylphenyl)sulfonyl]acrylamide

A mixture of (2E)-3-[1,3-dimethyl-5-(1H-pyrrolo[2,3-b]pyridin-1-yl)-1H-pyrazol-4-yl]acrylic acid obtained in Reference Example 13 (300 mg), 2-methyl-6-nitrobenzoic anhydride (440 mg), 4-methylbenzenesulfonamide (184 mg), triethylamine (329 mg), 4-dimethylaminopyridine (138 mg) and acetonitrile (8 mL) was stirred at room temperature for 16 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and the residue was crystallized from diisopropyl ether-methanol to give the title compound (420 mg, yield 91%) as colorless crystals. melting point 236.9-238.3° C.

¹H-NMR (300 MHz, DMSO-d₆) δ:2.35 (s, 3H), 2.35 (s, 3H), 3.47 (s, 3H), 6.04 (d, J=16.1 Hz, 1H), 6.86 (d, J=3.4 Hz, 1H), 6.93 (d, J=16.1 Hz, 1H), 7.27 (dd, J=8.0, 4.6 Hz, 1H), 7.37 (d, J=8.2 Hz, 2H), 7.67 (d, J=3.4 Hz, 1H), 7.74 (d, J=8.2 Hz, 2H), 8.15 (dd, J=8.0, 1.5 Hz, 1H), 8.25 (dd, J=4.6, 1.5 Hz, 1H), 12.01 (s, 1H).

### Example 34

### (2E)-3-[1,3-dimethyl-5-(1H-pyrrolo[2,3-b]pyridin-1-yl)-1H-pyrazol-4-yl]-2-methyl-N-(pentylsulfonyl)acrylamide

By a method similar to that in Example 1, the title compound was obtained from (2E)-3-[1,3-dimethyl-5-(1H-pyrrolo[2,3-b]pyridin-1-yl)-1H-pyrazol-4-yl]-2-methylacrylic acid obtained in Reference Example 48 and pentane-1-sulfonamide.

¹H-NMR (300 MHz, CDCl₃) δ:0.89 (t, J=7.2 Hz, 3H), 1.23 (s, 3H), 1.28-1.45 (m, 4H), 1.75-1.84 (m, 2H), 2.30 (s, 3H), 3.40-3.50 (m, 2H), 3.67 (s, 3H), 6.69 (d, J=3.8 Hz, 1H), 7.09 (d, J=3.8 Hz, 1H), 7.16-7.23 (m, 2H), 7.77 (s, 1H), 8.00 (dd, J=8.0, 1.5 Hz, 1H), 8.37 (dd, J=4.5, 1.5 Hz, 1H).

### Example 35

### (2E)-2-methyl-3-[1-methyl-5-(1H-pyrrolo[2,3-b]pyridin-1-yl)-1H-pyrazol-4-yl]-N-(pentylsulfonyl)acrylamide

By a method similar to that in Example 1, the title compound was obtained from (2E)-2-methyl-3-[1-methyl-5-(1H-pyrrolo[2,3-b]pyridin-1-yl)-1H-pyrazol-4-yl]acrylic acid obtained in Reference Example 53 and pentane-1-sulfonamide.

¹H-NMR (300 MHz, CDCl₃) δ:0.87 (t, J=7.1 Hz, 3H), 1.25-1.42 (m, 4H), 1.68-1.83 (m, 2H), 2.12 (s, 3H), 3.39-3.46 (m, 2H), 3.66 (s, 3H), 6.74-6.76 (m, 1H), 6.81 (s, 1H), 7.16-7.23 (m, 2H), 7.89 (s, 1H), 8.01 (d, J=7.9 Hz, 1H), 8.05-8.18 (m, 1H), 8.28-8.34 (m, 1H).

### Example 36

### (2E)-3-[5-(6-methoxy-2,3-dihydro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(6-methoxy-2,3-dihydro-1H-indol-1-yl)-1,3-dimethyl-1H-pyrazol-4-yl]acrylic acid obtained in Reference Example 57 and pentane-1-sulfonamide.

¹H-NMR (300 MHz, CDCl₃) δ:0.85-0.92 (m, 3H), 1.28-1.44 (m, 4H), 1.69-1.84 (m, 2H), 2.37 (s, 3H), 3.16-3.28 (m, 2H), 3.29-3.40 (m, 2H), 3.66 (s, 3H), 3.68 (s, 3H), 3.80-3.89 (m, 2H), 5.73 (d, J=2.3 Hz, 1H), 5.84 (d, J=15.5 Hz, 1H), 6.33 (dd, J=8.0, 2.3 Hz, 1H), 7.09 (d, J=8.0 Hz, 1H), 7.57 (d, J=15.5 Hz, 1H), 7.63-7.71 (m, 1H).

### Example 37

### potassium {(2E)-3-[5-(6-methoxy-2,3-dihydro-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(6-methoxy-2,3-dihydro-1H-indol-1-yl)-1,3-dimethyl-1H-pyrazol-4-yl]-N-(pentylsulfonyl)acrylamide obtained in Example 36.

¹H-NMR (300 MHz, DMSO-d₆) δ:0.80-0.87 (m, 3H), 1.16-1.30 (m, 4H), 1.45-1.55 (m, 2H), 2.25 (s, 3H), 2.85-2.95 (m, 2H), 3.10-3.17 (m, 2H), 3.55 (s, 3H), 3.59 (s, 3H), 3.70-3.80 (m, 1H), 3.81-3.92 (m, 1H), 5.58 (d, J=2.3 Hz, 1H), 5.86 (d, J=16.2 Hz, 1H), 6.25 (dd, J=8.0, 2.3 Hz, 1H), 6.94 (d, J=16.2 Hz, 1H), 7.05 (d, J=8.0 Hz, 1H).

### Example 38

### (2E)-3-[5-(6-methoxy-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(6-methoxy-1H-indol-1-yl)-1,3-dimethyl-1H-pyrazol-4-yl]acrylic acid obtained in Reference Example 60 and pentane-1-sulfonamide.

¹H-NMR (300 MHz, CDCl₃) δ:0.84-0.91 (m, 3H), 1.26-1.40 (m, 4H), 1.67-1.82 (m, 2H), 2.44 (s, 3H), 3.28-3.37 (m, 2H), 3.55 (s, 3H), 3.77 (s, 3H), 5.25 (d, J=15.6 Hz, 1H), 6.40 (d, J=2.3 Hz, 1H), 6.75 (dd, J=3.4, 0.8 Hz, 1H), 6.90 (dd, J=8.7, 2.3 Hz, 1H), 6.94 (d, J=3.4 Hz, 1H), 7.33 (s, 1H), 7.50 (d, J=15.6 Hz, 1H), 7.60 (d, J=8.7 Hz, 1H).

### Example 39

### potassium {(2E)-3-[5-(6-methoxy-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(6-methoxy-1H-indol-1-yl)-1,3-dimethyl-1H-pyrazol-4-yl]-N-(pentylsulfonyl)acrylamide obtained in Example 38.

¹H-NMR (300 MHz, DMSO-d₆) δ:0.76-0.86 (m, 3H), 1.15-1.26 (m, 4H), 1.40-1.48 (m, 2H), 2.34 (s, 3H), 2.79-2.91 (m, 2H), 3.42 (s, 3H), 3.69 (s, 3H), 5.60 (d, J=16.2 Hz, 1H), 6.44 (d, J=2.1 Hz, 1H), 6.71 (d, J=3.4 Hz, 1H), 6.74-6.86 (m, 2H), 7.33 (d, J=3.4 Hz, 1H), 7.57 (d, J=8.7 Hz, 1H).

### Example 40

### 3-[5-(2,3-dihydro-1H-pyrrolo[2,3-b]pyridin-1-yl)-1,3-dimethyl-1H-pyrazol-4-yl]-N-(pentylsulfonyl)propanamide

By a method similar to that in Example 1, the title compound was obtained from 3-[5-(2,3-dihydro-1H-pyrrolo[2,3-b]pyridin-1-yl)-1,3-dimethyl-1H-pyrazol-4-yl]propanoic acid obtained in Reference Example 46 and pentane-1-sulfonamide.

¹H-NMR (300 MHz, CDCl₃) δ:0.88-0.94 (m, 3H), 1.32-1.47 (m, 4H), 1.49-1.59 (m, 1H), 1.60-1.75 (m, 1H), 2.17 (s, 3H), 2.17-2.28 (m, 1H), 2.37-2.47 (m, 1H), 2.59-2.70 (m, 1H), 2.82-3.03 (m, 2H), 3.17-3.35 (m, 3H), 3.55 (s, 3H), 3.76-3.98 (m, 2H), 6.69 (dd, J=7.2, 5.5 Hz, 1H), 7.43 (d, J=7.2 Hz, 1H), 7.94 (dd, J=5.5, 1.5 Hz, 1H), 12.27 (s, 1H).

### Example 41

### N-[({2-[1,3-dimethyl-5-(1H-pyrrolo[2,3-b]pyridin-1-yl)-1H-pyrazol-4-yl]ethyl}amino)carbonyl]pentane-1-sulfonamide

3-[1,3-Dimethyl-5-(1H-pyrrolo[2,3-b]pyridin-1-yl)-1H-pyrazol-4-yl]propanoic acid obtained in Reference Example 45 (302 mg), diphenyl azidophosphate (438 mg) and triethylamine (165 mg) were dissolved in N,N-dimethylformamide (5 mL), and the solution was stirred at room temperature for 2 hr. Water was added to the reaction mixture, and the mixture was extracted with ethyl acetate. The organic layer was washed with saturated brine, dried over anhydrous magnesium sulfate, and filtrated. The filtrate was concentrated, and the residue was dissolved in toluene (8 mL). The solution was heated under reflux for 2 hr, 1,8-diazabicyclo[5.4.0]undec-7-ene (330 mg) and pentane-1-sulfonamide (165 mg) were added to the reaction mixture, and the mixture was further heated under reflux for 4 hr. The reaction mixture was concentrated under reduced pressure, and the residue was subjected to silica gel chromatography (ethyl acetate-methanol 95:5, v/v), and crystallized from hexane-ethanol to give the title compound (196 mg, yield 42%) as colorless crystals.

¹H-NMR (300 MHz, CDCl₃) δ:0.90 (t, J=7.1 Hz, 3H), 1.28-1.48 (m, 4H), 1.65-1.80 (m, 2H), 2.31 (s, 3H), 2.32-2.43 (m, 1H), 2.58-2.72 (m, 1H), 3.09-3.20 (m, 1H), 3.23-3.30 (m, 2H), 3.33-3.48 (m, 4H), 5.92 (s, 1H), 6.75 (d, J=3.8 Hz, 1H), 7.19 (d, J=3.8 Hz, 1H), 7.27 (dd, J=7.9, 4.7 Hz, 1H), 7.79 (s, 1H), 8.07 (dd, J=7.9, 1.5 Hz, 1H), 8.40 (dd, J=4.7, 1.5 Hz, 1H).

### Example 42

### N-[({2-[1,3-dimethyl-5-(1H-pyrrolo[2,3-b]pyridin-1-yl)-1H-pyrazol-4-yl]ethyl}amino)carbonyl]butane-1-sulfonamide

By a method similar to that in Example 41, the title compound was obtained from 3-[1,3-dimethyl-5-(1H-pyrrolo[2,3-b]pyridin-1-yl)-1H-pyrazol-4-yl]propanoic acid obtained in Reference Example 45 and butane-1-sulfonamide.

¹H-NMR (300 MHz, CDCl₃) δ:0.94 (t, J=7.3 Hz, 3H), 1.37-1.50 (m, 2H), 1.65-1.80 (m, 2H), 2.31 (s, 3H), 2.32-2.40 (m, 1H), 2.61-2.71 (m, 1H), 3.07-3.22 (m, 1H), 3.22-3.29 (m, 2H), 3.35-3.47 (m, 4H), 5.89 (s, 1H), 6.75 (d, J=3.6 Hz, 1H), 7.19 (d, J=3.6 Hz, 1H), 7.26 (dd, J=7.8, 4.8 Hz, 1H), 7.91 (s, 1H), 8.07 (dd, J=7.8, 1.5 Hz, 1H), 8.40 (dd, J=4.8, 1.5 Hz, 1H).

### Example 43

### (2E)-3-[5-(5-chloro-1H-indol-1-yl)-1,3-dimethyl-1H-pyrazol-4-yl]-N-(phenylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and benzenesulfonamide.

¹H-NMR (300 MHz, CDCl₃) δ:2.37 (s, 3H), 3.47 (s, 3H), 5.39 (d, J=15.9 Hz, 1H), 6.72 (d, J=3.0 Hz, 1H), 6.85 (d, J=8.7 Hz, 1H), 7.05 (d, J=3.4 Hz, 1H), 7.17 (dd, J=8.7, 1.9 Hz, 1H), 7.36 (d, J=15.5 Hz, 1H), 7.44-7.66 (m, 4H), 7.90-7.98 (m, 2H), 8.21 (br s, 1H).

### Example 44

### potassium {(2E)-3-[5-(5-chloro-1H-indol-1-yl)-1,3-dimethyl-1H-pyrazol-4-yl]prop-2-enoyl}(phenylsulfonyl)azanide

By a method similar to that in Example 7, the title compound was obtained from (2E)-3-[5-(5-chloro-1H-indol-1-yl)-1,3-dimethyl-1H-pyrazol-4-yl]-N-(phenylsulfonyl)acrylamide obtained in Example 43.

¹H-NMR (300 MHz, DMSO-d₆) δ:2.32 (s, 3H), 3.39 (s, 3H), 5.52 (d, J=15.9 Hz, 1H), 6.70 (d, J=16.3 Hz, 1H), 6.80 (d, J=3.4 Hz, 1H), 6.98 (d, J=8.7 Hz, 1H), 7.18 (dd, J=8.7, 1.9 Hz, 1H), 7.25-7.34 (m, 3H), 7.59 (d, J=3.4 Hz, 1H), 7.64-7.70 (m, 2H), 7.75 (d, J=1.9 Hz, 1H).

### Example 45

### (2E)-3-[5-(5-chloro-1H-indol-1-yl)-1,3-dimethyl-1H-pyrazol-4-yl]-N-[(4-methylphenyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 4-methylbenzenesulfonamide.

¹H-NMR (300 MHz, CDCl₃) δ:2.36 (s, 3H), 2.41 (s, 3H), 3.47 (s, 3H), 5.40 (d, J=15.6 Hz, 1H), 6.71 (d, J=3.2 Hz, 1H), 6.84 (d, J=8.9 Hz, 1H), 7.05 (d, J=3.4 Hz, 1H), 7.16 (dd, J=8.7, 1.9 Hz, 1H), 7.25-7.30 (m, 2H), 7.35 (d, J=15.6 Hz, 1H), 7.59 (d, J=1.9 Hz, 1H), 7.82 (d, J=8.3 Hz, 2H), 8.34 (br s, 1H).

### Example 46

### potassium {(2E)-3-[5-(5-chloro-1H-indol-1-yl)-1,3-dimethyl-1H-pyrazol-4-yl]prop-2-enoyl}[(4-methylphenyl)sulfonyl]azanide

By a method similar to that in Example 7, the title compound was obtained from (2E)-3-[5-(5-chloro-1H-indol-1-yl)-1,3-dimethyl-1H-pyrazol-4-yl]-N-[(4-methylphenyl)sulfonyl]acrylamide obtained in Example 45.

¹H-NMR (300 MHz, DMSO-d₆) δ:2.27 (s, 3H), 2.31 (s, 3H), 3.40 (s, 3H), 5.51 (d, J=16.3 Hz, 1H), 6.68 (d, J=16.3 Hz, 1H), 6.79 (d, J=3.0 Hz, 1H), 6.98 (d, J=8.7 Hz, 1H), 7.08 (d, J=8.3 Hz, 2H), 7.18 (dd, J=8.7, 1.9 Hz, 1H), 7.50-7.61 (m, 3H), 7.75 (d, J=1.9 Hz, 1H).

### Example 47

### (2E)-3-[5-(5-chloro-1H-indol-1-yl)-1,3-dimethyl-1H-pyrazol-4-yl]-N-[(2-chlorophenyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 2-chlorobenzenesulfonamide.

¹H-NMR (300 MHz, CDCl₃) δ:2.38 (s, 3H), 3.51 (s, 3H), 5.40 (d, J=15.8 Hz, 1H), 6.75 (dd, J=3.2, 0.8 Hz, 1H), 6.86 (d, J=8.9 Hz, 1H), 7.07 (d, J=3.2 Hz, 1H), 7.17-7.22 (m, 1H), 7.30-7.57 (m, 4H), 7.68 (d, J=1.7 Hz, 1H), 8.11 (dd, J=7.9, 1.5 Hz, 1H).

### Example 48

### (2E)-3-[5-(5-chloro-1H-indol-1-yl)-1,3-dimethyl-1H-pyrazol-4-yl]-N-[(3-chlorophenyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 3-chlorobenzenesulfonamide.

¹H-NMR (300 MHz, CDCl₃) δ:2.36 (s, 3H), 3.47 (s, 3H), 5.36 (d, J=15.8 Hz, 1H), 6.73 (d, J=3.2 Hz, 1H), 6.86 (d, J=8.7 Hz, 1H), 7.06 (d, J=3.4 Hz, 1H), 7.17 (dd, J=8.7, 2.1 Hz, 1H), 7.37 (d, J=15.6 Hz, 1H), 7.43 (d, J=7.9 Hz, 1H), 7.53-7.59 (m, 1H), 7.63 (d, J=1.9 Hz, 1H), 7.82-7.88 (m, 1H), 7.91-7.96 (m, 1H).

### Example 49

### (2E)-3-[5-(5-chloro-1H-indol-1-yl)-1,3-dimethyl-1H-pyrazol-4-yl]-N-[(4-chlorophenyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 4-chlorobenzenesulfonamide.

¹H-NMR (300 MHz, CDCl₃) δ:2.37 (s, 3H), 3.48 (s, 3H), 5.32 (d, J=15.8 Hz, 1H), 6.71-6.75 (m, 1H), 6.85 (d, J=8.7 Hz, 1H), 7.05 (d, J=3.2 Hz, 1H), 7.18 (dd, J=8.7, 1.9 Hz, 1H), 7.37 (d, J=15.8 Hz, 1H), 7.43-7.49 (m, 2H), 7.64 (d, J=1.9 Hz, 1H), 7.86-7.93 (m, 2H), 8.19 (br s, 1H).

### Example 50

### (2E)-3-[5-(5-chloro-1H-indol-1-yl)-1,3-dimethyl-1H-pyrazol-4-yl]-N-[(2,4-dichlorophenyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 2,4-dichlorobenzenesulfonamide.

¹H-NMR (300 MHz, CDCl₃) δ:2.37 (s, 3H), 3.48 (s, 3H), 6.71 (s, 1H), 6.87 (d, J=8.9 Hz, 1H), 7.07 (d, J=3.2 Hz, 1H), 7.14-7.45 (m, 5H), 7.64 (d, J=1.1 Hz, 1H), 7.91-8.06 (m, 1H).

### Example 51

### (2E)-3-[5-(5-chloro-1H-indol-1-yl)-1,3-dimethyl-1H-pyrazol-4-yl]-N-{[3-(trifluoromethyl)phenyl]sulfonyl}acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 3-(trifluoromethyl)benzenesulfonamide.

¹H-NMR (300 MHz, CDCl₃) δ:2.38 (s, 3H), 3.48 (s, 3H), 5.31 (d, J=15.6 Hz, 1H), 6.74 (dd, J=3.3, 0.8 Hz, 1H), 6.86 (d, J=8.7 Hz, 1H), 7.06 (d, J=3.4 Hz, 1H), 7.18 (dd, J=8.8, 2.0 Hz, 1H), 7.38 (d, J=15.6 Hz, 1H), 7.62-7.70 (m, 2H), 7.87 (d, J=7.5 Hz, 1H), 8.18-8.24 (m, 2H).

### Example 52

### (2E)-3-[5-(5-chloro-1H-indol-1-yl)-1,3-dimethyl-1H-pyrazol-4-yl]-N-{[4-(trifluoromethyl)phenyl]sulfonyl}acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 4-(trifluoromethyl)benzenesulfonamide.

¹H-NMR (300 MHz, CDCl₃) δ:2.37 (s, 3H), 3.48 (s, 3H), 5.29 (d, J=15.8 Hz, 1H), 6.74 (dd, J=3.2, 0.8 Hz, 1H), 6.85 (d, J=8.7 Hz, 1H), 7.06 (d, J=3.4 Hz, 1H), 7.18 (dd, J=8.7, 2.1 Hz, 1H), 7.39 (d, J=15.6 Hz, 1H), 7.65 (d, J=1.7 Hz, 1H), 7.76 (d, J=8.3 Hz, 2H), 8.09 (d, J=8.3 Hz, 2H).

### Example 53

### (2E)-3-[5-(5-chloro-1H-indol-1-yl)-1,3-dimethyl-1H-pyrazol-4-yl]-N-[(4-methoxyphenyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 4-methoxybenzenesulfonamide.

¹H-NMR (300 MHz, CDCl₃) δ:2.38 (s, 3H), 3.48 (s, 3H), 3.85 (s, 3 H), 5.33 (d, J=15.8 Hz, 1H), 6.75 (dd, J=3.4, 0.8 Hz, 1H), 6.86 (d, J=8.9 Hz, 1H), 6.90-6.96 (m, 2H), 7.06 (d, J=3.4 Hz, 1H), 7.19 (dd, J=8.7, 1.9 Hz, 1H), 7.36 (d, J=15.6 Hz, 1H), 7.67 (d, J=1.7 Hz, 1H), 7.84-7.90 (m, 2H).

### Example 54

### (2E)-N-[(4-butylphenyl)sulfonyl]-3-[5-(5-chloro-1H-indol-1-yl)-1,3-dimethyl-1H-pyrazol-4-yl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 4-butylbenzenesulfonamide.

¹H-NMR (300 MHz, CDCl₃) δ:0.89-0.95 (m, 3H), 1.24-1.43 (m, 2H), 1.53-1.66 (m, 2H), 2.37 (s, 3H), 2.61-2.70 (m, 2H), 3.48 (s, 3H), 5.38 (d, J=15.6 Hz, 1H), 6.73 (dd, J=3.2, 0.8 Hz, 1H), 6.85 (d, J=8.7 Hz, 1H), 7.05 (d, J=3.2 Hz, 1H), 7.17 (dd, J=8.7, 1.9 Hz, 1H), 7.25-7.31 (m, 2H), 7.36 (d, J=15.6 Hz, 1H), 7.64 (d, J=1.5 Hz, 1H), 7.80-7.88 (m, 2H).

### Example 55

### (2E)-3-[5-(5-chloro-1H-indol-1-yl)-1,3-dimethyl-1H-pyrazol-4-yl]-N-(2-furylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and furan-2-sulfonamide.

¹H-NMR (300 MHz, CDCl₃) δ:2.41 (s, 3H), 3.50 (s, 3H), 5.50 (d, J=15.8 Hz, 1H), 6.48 (dd, J=3.5, 1.8 Hz, 1H), 6.75-6.79 (m, 1H), 6.88 (d, J=8.7 Hz, 1H), 7.09 (d, J=3.4 Hz, 1H), 7.13 (d, J=3.4 Hz, 1H), 7.21 (dd, J=8.8, 2.0 Hz, 1H), 7.40 (d, J=15.6 Hz, 1H), 7.51 (d, J=0.8 Hz, 1H), 7.70 (d, J=1.9 Hz, 1H).

### Example 56

### (2E)-3-[5-(5-chloro-1H-indol-1-yl)-1,3-dimethyl-1H-pyrazol-4-yl]-N-(2-thienylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and thiophene-2-sulfonamide.

¹H-NMR (300 MHz, CDCl₃) δ:2.39 (s, 3H), 3.48 (s, 3H), 5.41 (d, J=15.6 Hz, 1H), 6.73-6.76 (m, 1H), 6.87 (d, J=8.7 Hz, 1H), 7.02-7.09 (m, 2H), 7.18 (dd, J=8.8, 2.0 Hz, 1H), 7.41 (d, J=15.8 Hz, 1H), 7.62 (dd, J=5.1, 1.3 Hz, 1H), 7.66 (d, J=1.7 Hz, 1H), 7.75 (dd, J=3.8, 1.3 Hz, 1H).

### Example 57

### (2E)-3-[5-(5-chloro-1H-indol-1-yl)-1,3-dimethyl-1H-pyrazol-4-yl]-N-(2,3-dihydro-1-benzofuran-5-ylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 2,3-dihydro-1-benzofuran-5-sulfonamide.

¹H-NMR (300 MHz, CDCl₃) δ:2.38 (s, 3H), 3.24 (t, J=8.8 Hz, 2H), 3.49 (s, 3H), 4.66 (t, J=8.8 Hz, 2H), 5.32 (d, J=15.8 Hz, 1H), 6.73-6.89 (m, 3H), 7.06 (d, J=3.4 Hz, 1H), 7.19 (dd, J=8.8, 2.0 Hz, 1H), 7.36 (d, J=15.8 Hz, 1H), 7.67 (d, J=1.7 Hz, 1H), 7.72 (dd, J=8.6, 2.2 Hz, 1H), 7.81 (d, J=1.9 Hz, 1H).

### Example 58

### (2E)-N-(benzylsulfonyl)-3-[5-(5-chloro-1H-indol-1-yl)-1,3-dimethyl-1H-pyrazol-4-yl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 1-phenylmethanesulfonamide.

¹H-NMR (300 MHz, CDCl₃) δ:2.38 (s, 3H), 3.47-3.52 (m, 3H), 4.49 (br s, 2H), 5.31 (d, J=15.3 Hz, 1H), 6.71 (d, J=2.1 Hz, 1H), 6.89 (d, J=8.7 Hz, 1H), 7.06 (d, J=3.2 Hz, 1H), 7.15-7.49 (m, 7H), 7.65 (s, 1H).

### Example 59

### (2E)-3-[5-(5-chloro-1H-indol-1-yl)-1,3-dimethyl-1H-pyrazol-4-yl]-N-(cyclopropylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and cyclopropanesulfonamide.

¹H-NMR (300 MHz, CDCl₃) δ:1.01-1.09 (m, 2H), 1.25-1.33 (m, 2H), 2.43 (s, 3H), 2.72-2.84 (m, 1H), 3.52 (s, 3H), 5.42 (d, J=15.8 Hz, 1H), 6.77 (d, J=3.2 Hz, 1H), 6.91 (d, J=8.9 Hz, 1H), 7.11 (d, J=3.2 Hz, 1H), 7.21 (dd, J=8.7, 1.9 Hz, 1H), 7.45 (d, J=15.8 Hz, 1H), 7.70 (d, J=1.9 Hz, 1H).

### Example 60

### (2E)-3-[5-(5-chloro-1H-indol-1-yl)-1,3-dimethyl-1H-pyrazol-4-yl]-N-[(cyclopropylmethyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 1-cyclopropylmethanesulfonamide obtained in Reference Example 100.

¹H-NMR (300 MHz, CDCl₃) δ:0.28 (d, J=4.5 Hz, 2H), 0.55-0.68 (m, 2H), 1.01 (d, J=8.0 Hz, 1H), 2.44 (s, 3H), 3.22 (dd, J=7.2, 1.9 Hz, 2H), 3.53 (s, 3H), 5.32 (d, J=15.5 Hz, 1H), 6.78 (d, J=3.4 Hz, 1H), 6.90 (d, J=8.7 Hz, 1H), 7.11 (d, J=3.4 Hz, 1H), 7.21 (dd, J=8.7, 1.9 Hz, 1H), 7.46 (d, J=15.9 Hz, 1H), 7.71 (s, 1H).

### Example 61

### (2E)-3-[5-(5-chloro-1H-indol-1-yl)-1,3-dimethyl-1H-pyrazol-4-yl]-N-[(4-methylpentyl)sulfonyl]acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 4-methylpentane-1-sulfonamide obtained in Reference Example 101.

¹H-NMR (300 MHz, CDCl₃) δ:0.86 (d, J=6.6 Hz, 6H), 1.18-1.29 (m, 2H), 1.45-1.58 (m, 1H), 1.66-1.80 (m, 2H), 2.44 (s, 3H), 3.30 (dd, J=8.7, 7.2 Hz, 2H), 3.52 (s, 3H), 5.33 (d, J=15.8 Hz, 1H), 6.78 (dd, J=3.3, 0.8 Hz, 1H), 6.88-6.93 (m, 1H), 7.10 (d, J=3.2 Hz, 1H), 7.22 (dd, J=8.7, 2.1 Hz, 1H), 7.45 (d, J=15.8 Hz, 1H), 7.71 (d, J=1.7 Hz, 1H).

### Example 62

### (2E)-3-[5-(5-chloro-1H-indol-1-yl)-1,3-dimethyl-1H-pyrazol-4-yl]-N-[(pentylamino)sulfonyl]acrylamide

A mixture of (2E)-3-[5-(5-chloro-1H-indol-1-yl)-1,3-dimethyl-1H-pyrazol-4-yl]acrylic acid obtained in Reference Example 38 (353 mg), 2-methyl-6-nitrobenzoic anhydride (462 mg), N-pentylsulfamide obtained in Reference Example 287 (195 mg), triethylamine (339 mg), 4-dimethylaminopyridine (137 mg) and acetonitrile (11 mL) was stirred at room temperature for 18 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 40:60, v/v), and crystallized from hexane-ethyl acetate to give the title compound (299 mg, yield 58%) as colorless crystals. melting point 184.3-184.4° C.

¹H-NMR (300 MHz, CDCl₃) δ:0.78-0.94 (m, 3H), 1.19-1.35 (m, 4H), 1.49 (d, J=6.4 Hz, 2H), 2.42 (s, 3H), 2.90 (q, J=6.6 Hz, 2H), 3.52 (s, 3H), 5.11-5.21 (m, 1H), 5.31 (d, J=15.5 Hz, 1H), 6.74-6.96 (m, 2H), 7.06-7.29 (m, 2H), 7.40 (d, J=15.9 Hz, 1H), 7.69 (s, 1H), 8.12 (br s, 1H).

### Example 63

### (2E)-3-[5-(5-chloro-1H-indol-1-yl)-1,3-dimethyl-1H-pyrazol-4-yl]-N-{[(2-isopropoxyethyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(2-isopropoxyethyl)sulfamide obtained in Reference Example 279.

¹H-NMR (300 MHz, CDCl₃) δ:1.10 (d, J=6.1 Hz, 6H), 2.43 (s, 3H), 3.13 (t, J=4.9 Hz, 2H), 3.42-3.57 (m, 6H), 5.26 (d, J=15.9 Hz, 1H), 5.44 (br s, 1H), 6.78 (d, J=3.4 Hz, 1H), 6.90 (d, J=8.7 Hz, 1H), 7.10 (d, J=3.0 Hz, 1H), 7.21 (dd, J=8.7, 1.9 Hz, 1H), 7.42 (d, J=15.9 Hz, 1H), 7.71 (d, J=1.9 Hz, 1H).

### Example 64

### (2E)-3-[5-(5-chloro-1H-indol-1-yl)-1,3-dimethyl-1H-pyrazol-4-yl]-N-{[methyl(pentyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-methyl-N-pentylsulfamide obtained in Reference Example 110.

¹H-NMR (300 MHz, CDCl₃) δ:0.79-0.92 (m, 3H), 1.19-1.40 (m, 4H), 1.47-1.60 (m, 2H), 2.42 (s, 3H), 2.87 (s, 3H), 3.20 (t, J=7.4 Hz, 2H), 3.50 (s, 3H), 5.41 (d, J=15.5 Hz, 1H), 6.76 (d, J=2.7 Hz, 1H), 6.89 (d, J=8.7 Hz, 1H), 7.09 (d, J=3.4 Hz, 1H), 7.20 (dd, J=8.7, 1.9 Hz, 1H), 7.38 (d, J=15.9 Hz, 1H), 7.68 (d, J=1.9 Hz, 1H), 8.13 (br s, 1H).

### Example 65

### [5-(5-chloro-1H-indol-1-yl)-1,3-dimethyl-1H-pyrazol-4-yl]methyl (pentylsulfonyl)carbamate

To a solution of [5-(5-chloro-1H-indol-1-yl)-1,3-dimethyl-1H-pyrazol-4-yl]methanol obtained in Reference Example 61 (358 mg) in N,N-dimethylformamide (13 mL) was added N,N′-carbonyldiimidazole (252 mg), and the mixture was stirred at 50° C. for 1 hr. Pentane-1-sulfonamide (294 mg), 1,8-diazabicyclo[5.4.0]undec-7-ene (336 mg) and 4-dimethylaminopyridine (206 mg) were added to this reaction mixture, and the mixture was stirred at 50° C. for 4 hr. After the reaction mixture was allowed to cool to room temperature, 1N hydrochloric acid was added, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 60:40, v/v), and crystallized from hexane-ethyl acetate to give the title compound (233 mg, yield 38%) as colorless crystals.

¹H-NMR (300 MHz, CDCl₃) δ:0.86-0.94 (m, 3H), 1.25-1.46 (m, 4H), 1.72-1.85 (m, 2H), 2.35 (s, 3H), 3.25-3.33 (m, 2H), 3.50 (s, 3H), 4.78-4.95 (m, 2H), 6.70 (dd, J=3.4, 0.8 Hz, 1H), 6.93 (d, J=8.7 Hz, 1H), 7.16 (d, J=3.4 Hz, 1H), 7.20 (dd, J=8.8, 2.0 Hz, 1H), 7.67 (d, J=1.7 Hz, 1H).

### Example 66

### 2-[5-(5-chloro-1H-indol-1-yl)-1,3-dimethyl-1H-pyrazol-4-yl]ethyl (pentylsulfonyl)carbamate

By a method similar to that in Example 65, the title compound was obtained from 2-[5-(5-chloro-1H-indol-1-yl)-1,3-dimethyl-1H-pyrazol-4-yl]ethanol obtained in Reference Example 63 and pentane-1-sulfonamide.

¹H-NMR (300 MHz, CDCl₃) δ:0.90 (t, J=7.0 Hz, 3H), 1.23-1.47 (m, 4H), 1.70-1.82 (m, 2H), 2.29 (s, 3H), 2.51-2.69 (m, 2H), 3.22-3.31 (m, 2H), 3.49 (s, 3H), 3.92-4.17 (m, 2H), 6.71 (d, J=3.4 Hz, 1H), 6.95 (d, J=8.7 Hz, 1H), 7.13 (d, J=3.4 Hz, 1H), 7.18-7.27 (m, 1H), 7.69 (d, J=2.3 Hz, 1H).

### Example 67

### 3-[5-(5-chloro-1H-indol-1-yl)-1,3-dimethyl-1H-pyrazol-4-yl]propyl (pentylsulfonyl)carbamate

By a method similar to that in Example 65, the title compound was obtained from 3-[5-(5-chloro-1H-indol-1-yl)-1,3-dimethyl-1H-pyrazol-4-yl]propan-1-ol obtained in Reference Example 66 and pentane-1-sulfonamide.

¹H-NMR (300 MHz, CDCl₃) δ:0.86-0.94 (m, 3H), 1.28-1.47 (m, 4H), 1.56-1.70 (m, 2H), 1.73-1.86 (m, 2H), 2.20-2.42 (m, 5H), 3.25-3.33 (m, 2H), 3.47 (s, 3H), 3.93-4.03 (m, 2H), 6.69-6.72 (m, 1H), 6.94 (d, J=8.7 Hz, 1H), 7.11 (d, J=3.2 Hz, 1H), 7.17-7.22 (m, 1H), 7.68 (d, J=1.7 Hz, 1H).

### Example 68

### [5-(5-chloro-1H-indol-1-yl)-1,3-dimethyl-1H-pyrazol-4-yl]methyl (phenylsulfonyl)carbamate

By a method similar to that in Example 65, the title compound was obtained from [5-(5-chloro-1H-indol-1-yl)-1,3-dimethyl-1H-pyrazol-4-yl]methanol obtained in Reference Example 61 and benzenesulfonamide.

¹H-NMR (300 MHz, CDCl₃) δ:2.24 (s, 3H), 3.49 (s, 3H), 4.65-4.83 (m, 2H), 6.66 (d, J=2.8 Hz, 1H), 6.85 (d, J=8.7 Hz, 1H), 7.08 (d, J=3.2 Hz, 1H), 7.15 (dd, J=8.8, 2.0 Hz, 1H), 7.51 (t, J=7.7 Hz, 2H), 7.60-7.68 (m, 2H), 7.91-7.98 (m, 2H).

### Example 69

### [5-(5-chloro-1H-indol-1-yl)-1,3-dimethyl-1H-pyrazol-4-yl]methyl [(4-methylphenyl)sulfonyl]carbamate

By a method similar to that in Example 65, the title compound was obtained from [5-(5-chloro-1H-indol-1-yl)-1,3-dimethyl-1H-pyrazol-4-yl]methanol obtained in Reference Example 61 and 4-methylbenzenesulfonamide.

¹H-NMR (300 MHz, CDCl₃) δ:2.25 (s, 3H), 2.44 (s, 3H), 3.49 (s, 3H), 4.64-4.81 (m, 2H), 6.66 (d, J=2.6 Hz, 1H), 6.86 (d, J=8.7 Hz, 1H), 7.08 (d, J=3.2 Hz, 1H), 7.15 (dd, J=8.7, 1.9 Hz, 1H), 7.29 (d, J=8.3 Hz, 2H), 7.65 (d, J=1.7 Hz, 1H), 7.82 (d, J=8.5 Hz, 2H).

### Example 70

### [5-(5-chloro-1H-indol-1-yl)-1,3-dimethyl-1H-pyrazol-4-yl]methyl [(4-methoxyphenyl)sulfonyl]carbamate

By a method similar to that in Example 65, the title compound was obtained from [5-(5-chloro-1H-indol-1-yl)-1,3-dimethyl-1H-pyrazol-4-yl]methanol obtained in Reference Example 61 and 4-methoxybenzenesulfonamide.

¹H-NMR (300 MHz, CDCl₃) δ:2.26 (s, 3H), 3.49 (s, 3H), 3.87 (s, 3H), 4.65-4.83 (m, 2H), 6.64-6.69 (m, 1H), 6.84-7.02 (m, 3H), 7.09 (d, J=3.4 Hz, 1H), 7.16 (dd, J=8.7, 1.9 Hz, 1H), 7.65 (d, J=1.9 Hz, 1H), 7.83-7.90 (m, 2H).

### Example 71

### [5-(5-chloro-1H-indol-1-yl)-1,3-dimethyl-1H-pyrazol-4-yl]methyl {[(2-isopropoxyethyl)amino]sulfonyl}carbamate

To a solution of [5-(5-chloro-1H-indol-1-yl)-1,3-dimethyl-1H-pyrazol-4-yl]methanol obtained in Reference Example 61 (355 mg) in acetonitrile (13 mL) was added chlorosulfonyl isocyanate (191 mg) with stirring at 0° C., and the mixture was stirred at 0° C. for 30 min. Pyridine (306 mg) was added to this reaction mixture, and the mixture stirred at 0° C. for 1 hr. 2-Aminoethyl isopropyl ether (797 mg) was added, and the mixture was stirred at room temperature for 17 hr. Water was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 40:60, v/v) to give the title compound (141 mg, yield 23%) as a colorless amorphous solid.

¹H-NMR (300 MHz, CDCl₃) δ:1.09 (dd, J=6.0, 0.9 Hz, 6H), 2.36 (s, 3H), 3.17 (br s, 2H), 3.44-3.56 (m, 6H), 4.77-4.92 (m, 2H), 5.35 (br s, 1H), 6.70 (dd, J=3.4, 0.8 Hz, 1H), 6.94 (d, J=8.7 Hz, 1H), 7.15-7.23 (m, 2H), 7.67 (d, J=1.5 Hz, 1H).

### Example 72

### 2-[5-(5-chloro-1H-indol-1-yl)-1,3-dimethyl-1H-pyrazol-4-yl]ethyl {[(2-isopropoxyethyl)amino]sulfonyl}carbamate

By a method similar to that in Example 71, the title compound was obtained from 2-[5-(5-chloro-1H-indol-1-yl)-1,3-dimethyl-1H-pyrazol-4-yl]ethanol obtained in Reference Example 63, chlorosulfonyl isocyanate and 2-aminoethyl isopropyl ether.

¹H-NMR (300 MHz, CDCl₃) δ:1.10 (d, J=6.2 Hz, 6H), 2.31 (s, 3H), 2.50-2.68 (m, 2H), 3.08-3.18 (m, 2H), 3.45-3.58 (m, 6H), 3.93-4.17 (m, 2H), 5.34 (br s, 1H), 6.71 (dd, J=3.2, 0.8 Hz, 1H), 6.96 (d, J=8.7 Hz, 1H), 7.14 (d, J=3.4 Hz, 1H), 7.22 (dd, J=8.9, 2.1 Hz, 1H), 7.69 (d, J=1.5 Hz, 1H).

### Example 73

### 3-[5-(5-chloro-1H-indol-1-yl)-1,3-dimethyl-1H-pyrazol-4-yl]propyl {[(2-isopropoxyethyl)amino]sulfonyl}carbamate

By a method similar to that in Example 71, the title compound was obtained from 3-[5-(5-chloro-1H-indol-1-yl)-1,3-dimethyl-1H-pyrazol-4-yl]propan-1-ol obtained in Reference Example 66, chlorosulfonyl isocyanate and 2-aminoethyl isopropyl ether.

¹H-NMR (300 MHz, CDCl₃) δ:1.12 (dd, J=6.1, 1.0 Hz, 6H), 1.52-1.68 (m, 2H), 2.19-2.41 (m, 5H), 3.17 (br s, 2H), 3.44-3.62 (m, 6H), 3.90-4.04 (m, 2H), 5.34 (br s, 1H), 6.71 (dd, J=3.4, 0.8 Hz, 1H), 6.94 (d, J=8.7 Hz, 1H), 7.11 (d, J=3.2 Hz, 1H), 7.17-7.22 (m, 1H), 7.68 (d, J=1.7 Hz, 1H).

### Example 74

### 3-[5-(5-chloro-1H-indol-1-yl)-1,3-dimethyl-1H-pyrazol-4-yl]propyl {[(4-methoxybenzyl)amino]sulfonyl}carbamate

By a method similar to that in Example 71, the title compound was obtained from 3-[5-(5-chloro-1H-indol-1-yl)-1,3-dimethyl-1H-pyrazol-4-yl]propan-1-ol obtained in Reference Example 66, chlorosulfonyl isocyanate and 4-methoxybenzylamine.

¹H-NMR (300 MHz, CDCl₃) δ:1.50-1.62 (m, 2H), 2.16-2.37 (m, 5H), 3.47 (s, 3H), 3.76 (s, 3H), 3.82-3.95 (m, 2H), 4.12 (q, J=7.2 Hz, 2H), 5.30 (br s, 1H), 6.69 (dd, J=3.2, 0.8 Hz, 1H), 6.79-6.85 (m, 2H), 6.93 (d, J=8.7 Hz, 1H), 7.10 (d, J=3.2 Hz, 1H), 7.14-7.22 (m, 3H), 7.68 (d, J=1.9 Hz, 1H).

### Example 75

### 2-[5-(5-chloro-1H-indol-1-yl)-1,3-dimethyl-1H-pyrazol-4-yl]-N-(pentylsulfonyl)acetamide

By a method similar to that in Example 1, the title compound was obtained from [5-(5-chloro-1H-indol-1-yl)-1,3-dimethyl-1H-pyrazol-4-yl]acetic acid obtained in Reference Example 67 and pentane-1-sulfonamide.

¹H-NMR (300 MHz, CDCl₃) δ:0.86-0.93 (m, 3H), 1.19-1.38 (m, 4H), 1.52-1.70 (m, 2H), 2.29 (s, 3H), 3.09-3.32 (m, 4H), 3.52 (s, 3H), 6.70 (dd, J=3.4, 0.8 Hz, 1H), 6.95 (d, J=8.7 Hz, 1H), 7.13 (d, J=3.2 Hz, 1H), 7.21 (dd, J=8.7, 2.1 Hz, 1H), 7.67 (d, J=1.5 Hz, 1H).

### Example 76

### 3-[5-(5-chloro-1H-indol-1-yl)-1,3-dimethyl-1H-pyrazol-4-yl]-N-(pentylsulfonyl)propanamide

By a method similar to that in Example 1, the title compound was obtained from 3-[5-(5-chloro-1H-indol-1-yl)-1,3-dimethyl-1H-pyrazol-4-yl]propanoic acid obtained in Reference Example 68 and pentane-1-sulfonamide.

¹H-NMR (300 MHz, CDCl₃) δ:0.85-0.93 (m, 3H), 1.21-1.43 (m, 4H), 1.64-1.77 (m, 2H), 2.10-2.19 (m, 2H), 2.29 (s, 3H), 2.53-2.66 (m, 2H), 3.24-3.32 (m, 2H), 3.44 (s, 3H), 6.69 (dd, J=3.3, 0.8 Hz, 1H), 6.89-6.94 (m, 1H), 7.12 (d, J=3.2 Hz, 1H), 7.20 (dd, J=8.7, 2.1 Hz, 1H), 7.67 (d, J=1.7 Hz, 1H).

### Example 77

### (2E)-4-[5-(5-chloro-1H-indol-1-yl)-1,3-dimethyl-1H-pyrazol-4-yl]-N-(pentylsulfonyl)but-2-enamide

By a method similar to that in Example 1, the title compound was obtained from (2E)-4-[5-(5-chloro-1H-indol-1-yl)-1,3-dimethyl-1H-pyrazol-4-yl]but-2-ene acid obtained in Reference Example 70 and pentane-1-sulfonamide.

¹H-NMR (300 MHz, CDCl₃) δ:0.86-0.94 (m, 3H), 1.22-1.45 (m, 4H), 1.67-1.81 (m, 2H), 2.39 (s, 3H), 3.02 (dd, J=7.3, 1.1 Hz, 2H), 3.28-3.37 (m, 2H), 3.51 (s, 3H), 5.15-5.27 (m, 1H), 6.09 (d, J=16.2 Hz, 1H), 6.72-6.76 (m, 1H), 6.90-6.99 (m, 1H), 7.09-7.14 (m, 1H), 7.22 (dd, J=8.7, 2.1 Hz, 1H), 7.68 (d, J=1.9 Hz, 1H).

### Example 78

### 2-[5-(5-chloro-1H-indol-1-yl)-1,3-dimethyl-1H-pyrazol-4-yl]-N-[(pentylamino)sulfonyl]acetamide

By a method similar to that in Example 62, the title compound was obtained from [5-(5-chloro-1H-indol-1-yl)-1,3-dimethyl-1H-pyrazol-4-yl]acetic acid obtained in Reference Example 67 and N-pentylsulfamide obtained in Reference Example 287.

¹H-NMR (300 MHz, CDCl₃) δ:0.88 (t, J=6.9 Hz, 3H), 1.16-1.33 (m, 4H), 1.37-1.49 (m, 2H), 2.28 (s, 3H), 2.71 (d, J=6.2 Hz, 2H), 3.14-3.31 (m, 2H), 3.51 (s, 3H), 5.08 (br s, 1H), 6.67-6.71 (m, 1H), 6.95 (d, J=8.7 Hz, 1H), 7.13 (d, J=3.4 Hz, 1H), 7.20 (dd, J=8.7, 1.9 Hz, 1H), 7.66 (d, J=1.7 Hz, 1H).

### Example 79

### 4-[5-(5-chloro-1H-indol-1-yl)-1,3-dimethyl-1H-pyrazol-4-yl]-N-[(pentylamino)sulfonyl]butanamide

By a method similar to that in Example 62, the title compound was obtained from 4-[5-(5-chloro-1H-indol-1-yl)-1,3-dimethyl-1H-pyrazol-4-yl]butanoic acid obtained in Reference Example 71 and N-pentylsulfamide obtained in Reference Example 287.

¹H-NMR (300 MHz, CDCl₃) δ:0.88 (t, J=6.6 Hz, 3H), 1.24-1.32 (m, 4H), 1.43-1.67 (m, 4H), 2.00-2.09 (m, 2H), 2.16-2.36 (m, 5H), 2.81-2.91 (m, 2H), 3.48 (s, 3H), 5.05 (br s, 1H), 6.70 (d, J=3.0 Hz, 1H), 6.94 (d, J=8.7 Hz, 1H), 7.11 (d, J=3.4 Hz, 1H), 7.20 (dd, J=8.7, 1.9 Hz, 1H), 7.67 (d, J=1.5 Hz, 1H).

### Example 80

### N-[({[5-(5-chloro-1H-indol-1-yl)-1,3-dimethyl-1H-pyrazol-4-yl]methyl}amino)carbonyl]pentane-1-sulfonamide

To a solution of 1-[5-(5-chloro-1H-indol-1-yl)-1,3-dimethyl-1H-pyrazol-4-yl]methanamine obtained in Reference Example 75 (508 mg) in N,N-dimethylformamide (18 mL) was added N,N′-carbonyldiimidazole (449 mg), and the mixture was stirred at 50° C. for 2 hr. Pentane-1-sulfonamide (419 mg), 1,8-diazabicyclo[5.4.0]undec-7-ene (478 mg) and 4-dimethylaminopyridine (384 mg) were added to this reaction mixture, and the mixture was stirred at 50° C. for 4 hr. After the reaction mixture was allowed to cool to room temperature, 1N hydrochloric acid was added, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 40:60, v/v), and crystallized from hexane-ethyl acetate to give the title compound (612 mg, yield 73%) as colorless crystals.

¹H-NMR (300 MHz, CDCl₃) δ:0.86-0.93 (m, 3H), 1.23-1.38 (m, 4H), 1.66-1.78 (m, 2H), 2.33 (s, 3H), 2.92-3.00 (m, 2H), 3.47 (s, 3H), 3.99-4.12 (m, 2H), 6.33-6.42 (m, 1H), 6.69 (d, J=3.0 Hz, 1H), 6.93 (d, J=8.7 Hz, 1H), 7.16 (d, J=3.4 Hz, 1H), 7.20 (dd, J=8.7, 1.9 Hz, 1H), 7.66 (d, J=1.9 Hz, 1H).

### Example 81

### N-[({2-[5-(5-chloro-1H-indol-1-yl)-1,3-dimethyl-1H-pyrazol-4-yl]ethyl}amino)carbonyl]pentane-1-sulfonamide

By a method similar to that in Example 80, the title compound was obtained from 2-[5-(5-chloro-1H-indol-1-yl)-1,3-dimethyl-1H-pyrazol-4-yl]ethanamine obtained in Reference Example 76 and pentane-1-sulfonamide.

¹H-NMR (300 MHz, CDCl₃) δ:0.89 (t, J=7.0 Hz, 3H), 1.24-1.45 (m, 4H), 1.70-1.83 (m, 2H), 2.30 (s, 3H), 2.34-2.57 (m, 2 H), 3.06-3.18 (m, 4H), 3.46 (s, 3H), 6.31 (br s, 1H), 6.68 (d, J=3.2 Hz, 1H), 6.93 (d, J=8.7 Hz, 1H), 7.15-7.22 (m, 2H), 7.65 (d, J=1.9 Hz, 1H)

### Example 82

### N-[({3-[5-(5-chloro-1H-indol-1-yl)-1,3-dimethyl-1H-pyrazol-4-yl]propyl}amino)carbonyl]pentane-1-sulfonamide

By a method similar to that in Example 80, the title compound was obtained from 3-[5-(5-chloro-1H-indol-1-yl)-1,3-dimethyl-1H-pyrazol-4-yl]propan-1-amine obtained in Reference Example 77 and pentane-1-sulfonamide.

¹H-NMR (300 MHz, CDCl₃) δ:0.86-0.93 (m, 3H), 1.29-1.40 (m, 4 H), 1.42-1.54 (m, 2H), 1.70-1.82 (m, 2H), 2.14-2.34 (m, 5H), 3.02-3.12 (m, 4H), 3.45 (s, 3H), 6.24 (t, J=5.3 Hz, 1H), 6.68 (dd, J=3.3, 0.8 Hz, 1H), 6.93 (d, J=8.7 Hz, 1H), 7.11 (d, J=3.2 Hz, 1H), 7.18 (dd, J=8.7, 1.9 Hz, 1H), 7.66 (d, J=1.7 Hz, 1H).

### Example 83

### N-[({[5-(5-chloro-1H-indol-1-yl)-1,3-dimethyl-1H-pyrazol-4-yl]methyl}amino)carbonyl]-4-methylbenzenesulfonamide

By a method similar to that in Example 80, the title compound was obtained from 1-[5-(5-chloro-1H-indol-1-yl)-1,3-dimethyl-1H-pyrazol-4-yl]methanamine obtained in Reference Example 75 and 4-methylbenzenesulfonamide.

¹H-NMR (300 MHz, CDCl₃) δ:2.23 (s, 3H), 2.42 (s, 3H), 3.48 (s, 3H), 3.98 (d, J=4.9 Hz, 2H), 6.56 (br s, 1H), 6.68 (d, J=3.0 Hz, 1H), 6.92 (d, J=8.7 Hz, 1H), 7.11-7.30 (m, 4H), 7.53-7.70 (m, 3H).

### Example 84

### (5Z)-5-{[5-(5-chloro-1H-indol-1-yl)-1,3-dimethyl-1H-pyrazol-4-yl]methylene}-1,3-thiazolidine-2,4-dione

To a solution of 5-(5-chloro-1H-indol-1-yl)-1,3-dimethyl-1H-pyrazole-4-carbaldehyde obtained in Reference Example 37 (560 mg) in ethanol (6.8 mL) were added 1,3-thiazolidine-2,4-dione (719 mg) and piperidine (382 mg), and the mixture was heated under reflux for 20 hr. After the reaction mixture was allowed to cool to room temperature, 1N hydrochloric acid was added, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 70:30, v/v), and crystallized from hexane-ethyl acetate to give the title compound (213 mg, yield 28%) as colorless crystals.

¹H-NMR (300 MHz, CDCl₃) δ:2.43 (s, 3H), 3.45 (s, 3H), 6.76 (d, J=3.4 Hz, 1H), 6.91 (d, J=8.7 Hz, 1H), 7.12 (d, J=3.0 Hz, 1H), 7.22 (dd, J=8.7, 1.9 Hz, 1H), 7.64 (s, 1H), 7.67 (d, J=1.9 Hz, 1H), 8.07 (br s, 1H).

### Example 85

### (5Z)-5-{3-[5-(5-chloro-1H-indol-1-yl)-1,3-dimethyl-1H-pyrazol-4-yl]propylidene}-1,3-thiazolidine-2,4-dione

By a method similar to that in Example 84, the title compound was obtained from 3-[5-(5-chloro-1H-indol-1-yl)-1,3-dimethyl-1H-pyrazol-4-yl]propanal obtained in Reference Example 78 and 1,3-thiazolidine-2,4-dione.

¹H-NMR (300 MHz, CDCl₃) δ:2.14 (q, J=7.3 Hz, 2H), 2.30 (s, 3H), 2.37-2.57 (m, 2H), 3.48 (s, 3H), 6.70 (d, J=3.2 Hz, 1H), 6.76 (t, J=7.6 Hz, 1H), 6.91 (d, J=8.7 Hz, 1H), 7.09 (d, J=3.2 Hz, 1H), 7.19 (dd, J=8.7, 1.9 Hz, 1H), 7.67 (d, J=1.7 Hz, 1H), 8.20 (br s, 1H).

### Example 86

### 5-{[5-(5-chloro-1H-indol-1-yl)-1,3-dimethyl-1H-pyrazol-4-yl]methyl}-1,3-thiazolidine-2,4-dione

(5Z)-5-{[5-(5-Chloro-1H-indol-1-yl)-1,3-dimethyl-1H-pyrazol-4-yl]methylene}-1,3-thiazolidine-2,4-dione obtained in Example 84 (86 mg) was dissolved in a mixed solvent of tetrahydrofuran (2 mL) and ethanol (2 mL), 10% palladium carbon (30 mg) was added, and the mixture was stirred under 1 atom of hydrogen atmosphere at room temperature for 6 days. The catalyst was removed by filtration, and the filtrate was concentrated. The residue was subjected to silica gel column chromatography (hexane-ethyl acetate 50:50, v/v), and crystallized from hexane-ethanol to give the title compound (33 mg, yield 37%) as colorless crystals.

¹H-NMR (300 MHz, DMSO-d₆) δ: 2.50 (br s, 3H), 2.67 (br s, 1H), 3.13 (br s, 1H), 3.38 (s, 3H), 4.29 (br s, 1H), 6.77 (br s, 1H), 6.96-7.10 (m, 1H), 7.20 (d, J=7.2 Hz, 1H), 7.59 (br s, 1H), 7.75 (br s, 1H), 11.94 (d, J=1.5 Hz, 1H).

### Example 87

### 5-{3-[5-(5-chloro-1H-indol-1-yl)-1,3-dimethyl-1H-pyrazol-4-yl]propyl}-1,3-thiazolidine-2,4-dione

By a method similar to that in Example 86, the title compound was obtained from (5Z)-5-{3-[5-(5-chloro-1H-indol-1-yl)-1,3-dimethyl-1H-pyrazol-4-yl]propylidene}-1,3-thiazolidine-2,4-dione obtained in Example 85.

¹H-NMR (300 MHz, CDCl₃) δ:1.39 (d, J=0.9 Hz, 2H), 1.64-1.78 (m, 1H), 1.93 (br s, 1H), 2.20-2.36 (m, 5H), 3.47 (d, J=2.4 Hz, 3H), 4.02-4.10 (m, 1H), 6.68 (d, J=3.2 Hz, 1H), 6.93 (d, J=8.7 Hz, 1H), 7.08 (dd, J=3.2, 1.3 Hz, 1H), 7.16-7.22 (m, 1H), 7.67 (s, 1H), 8.02 (br s, 1H).

### Example 88

### (2E)-3-[5-(5-cyano-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(5-cyano-1H-indol-1-yl)-1,3-dimethyl-1H-pyrazol-4-yl]acrylic acid obtained in Reference Example 81 and pentane-1-sulfonamide.

¹H-NMR (300 MHz, CDCl₃) δ:0.84-0.91 (m, 3H), 1.23-1.46 (m, 4H), 1.68-1.80 (m, 2H), 2.46 (s, 3H), 3.29-3.38 (m, 2H), 3.53 (s, 3H), 5.45 (d, J=15.6 Hz, 1H), 6.93 (d, J=3.2 Hz, 1H), 7.07 (d, J=8.5 Hz, 1H), 7.23 (d, J=3.4 Hz, 1H), 7.40 (d, J=15.8 Hz, 1H), 7.51 (dd, J=8.5, 1.3 Hz, 1H), 8.10 (d, J=0.9 Hz, 1H).

### Example 89

### (2E)-3-[5-(6-fluoro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(6-fluoro-1H-indol-1-yl)-1,3-dimethyl-1H-pyrazol-4-yl]acrylic acid obtained in Reference Example 83 and pentane-1-sulfonamide.

¹H-NMR (300 MHz, CDCl₃) δ:0.83-0.90 (m, 3H), 1.19-1.44 (m, 4H), 1.66-1.79 (m, 2H), 2.44 (s, 3H), 3.29-3.38 (m, 2H), 3.53 (s, 3H), 5.40 (d, J=15.8 Hz, 1H), 6.67 (dd, J=9.1, 2.4 Hz, 1H), 6.80 (dd, J=3.3, 0.8 Hz, 1H), 6.97-7.08 (m, 2H), 7.45 (d, J=15.8 Hz, 1H), 7.65 (dd, J=8.8, 5.2 Hz, 1H).

### Example 90

### potassium {(2E)-3-[5-(6-fluoro-1H-indol-1-yl)-1,3-dimethyl-1H-pyrazol-4-yl]prop-2-enoyl}(pentylsulfonyl)azanide

By a method similar to that in Example 7, the title compound was obtained from (2E)-3-[5-(6-fluoro-1H-indol-1-yl)-1,3-dimethyl-1H-pyrazol-4-yl]-N-(pentylsulfonyl)acrylamide obtained in Example 89.

¹H-NMR (300 MHz, DMSO-d₆) δ: 0.76-0.85 (m, 3H), 1.15-1.26 (m, 4H), 1.37-1.49 (m, 2H), 2.34 (s, 3H), 2.81-2.91 (m, 2H), 3.43 (s, 3H), 5.55 (d, J=15.9 Hz, 1H), 6.71-6.85 (m, 3H), 6.99-7.09 (m, 1H), 7.52 (d, J=3.0 Hz, 1H), 7.71 (dd, J=8.7, 5.3 Hz, 1H).

### Example 91

### (2E)-3-[5-(5-fluoro-1H-indol-1-yl)-3-(methoxymethyl)-1-methyl-1H-pyrazol-4-yl]-N-(pentylsulfonyl)acrylamide

By a method similar to that in Example 1, the title compound was obtained from (2E)-3-[5-(5-fluoro-1H-indol-1-yl)-3-(methoxymethyl)-1-methyl-1H-pyrazol-4-yl]acrylic acid obtained in Reference Example 88 and pentane-1-sulfonamide.

¹H-NMR (300 MHz, CDCl₃) δ:0.83-0.91 (m, 3H), 1.25-1.41 (m, 4H), 1.68-1.81 (m, 2H), 3.30-3.39 (m, 2H), 3.46 (s, 3H), 3.57 (s, 3H), 4.54-4.65 (m, 2H), 5.76 (d, J=15.8 Hz, 1H), 6.79 (d, J=2.8 Hz, 1H), 6.86-6.93 (m, 1H), 6.95-7.04 (m, 1H), 7.13 (d, J=3.4 Hz, 1H), 7.34-7.47 (m, 2H).

### Example 92

### 3-{(E)-2-[5-(5-chloro-1H-indol-1-yl)-1,3-dimethyl-1H-pyrazol-4-yl]vinyl}-1,2,4-oxadiazol-5(4H)-one

To a solution of (1Z,2E)-3-[5-(5-chloro-1H-indol-1-yl)-1,3-dimethyl-1H-pyrazol-4-yl]-N′-hydroxyprop-2-enimidamide obtained in Reference Example 90 (152 mg) in tetrahydrofuran (4.6 mL) were added N,N′-carbonyldiimidazole (112 mg) and 1,8-diazabicyclo[5.4.0]undec-7-ene (281 mg), and the mixture was stirred at room temperature for 4 hr. 1N Hydrochloric acid was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 40:60, v/v), and crystallized from hexane-ethyl acetate to give the title compound (109 mg, yield 67%) as colorless crystals.

¹H-NMR (300 MHz, CDCl₃) δ:2.47 (s, 3H), 3.54 (s, 3H), 5.75 (d, J=17.0 Hz, 1H), 6.73 (d, J=17.0 Hz, 1H), 6.77 (d, J=3.2 Hz, 1H), 6.91 (d, J=8.7 Hz, 1H), 7.12 (d, J=3.4 Hz, 1H), 7.20 (dd, J=8.6, 2.0 Hz, 1H), 7.69 (d, J=1.9 Hz, 1H).

### Example 93

### 2-{[5-(5-chloro-1H-indol-1-yl)-1,3-dimethyl-1H-pyrazol-4-yl]methoxy}-N-(pentylsulfonyl)acetamide

By a method similar to that in Example 1, the title compound was obtained from {[5-(5-chloro-1H-indol-1-yl)-1,3-dimethyl-1H-pyrazol-4-yl]methoxy}acetic acid obtained in Reference Example 93 and pentane-1-sulfonamide.

¹H-NMR (300 MHz, CDCl₃) δ:0.87-0.94 (m, 3H), 1.27-1.45 (m, 4H), 1.73-1.86 (m, 2H), 2.35 (s, 3H), 3.31-3.40 (m, 2H), 3.53 (s, 3H), 3.82 (s, 2H), 4.14-4.32 (m, 2H), 6.73 (d, J=3.4 Hz, 1H), 6.94 (d, J=8.7 Hz, 1H), 7.13 (d, J=3.0 Hz, 1H), 7.22 (dd, J=8.7, 1.9 Hz, 1H), 7.68 (d, J=1.9 Hz, 1H).

### Example 94

### (2E)-3-[5-(5-chloro-1H-indol-1-yl)-1,3-dimethyl-1H-pyrazol-4-yl]-N-{[(1-propylbutyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(1-propylbutyl)sulfamide obtained in Reference Example 112.

¹H-NMR (300 MHz, CDCl₃) δ:0.82 (t, J=7.0 Hz, 6H), 1.18-1.46 (m, 8H), 2.43 (s, 3H), 3.20-3.31 (m, 1H), 3.53 (s, 3H), 4.86 (d, J=8.0 Hz, 1H), 5.27 (d, J=15.5 Hz, 1H), 6.78 (d, J=3.4 Hz, 1H), 6.90 (d, J=8.7 Hz, 1H), 7.10 (d, J=3.4 Hz, 1H), 7.20 (dd, J=8.7, 1.9 Hz, 1H), 7.41 (d, J=15.9 Hz, 1H), 7.71 (d, J=1.9 Hz, 1H), 7.83 (br s, 1H).

### Example 95

### (2E)-3-[5-(5-chloro-1H-indol-1-yl)-1,3-dimethyl-1H-pyrazol-4-yl]-N-[(cyclohexylamino)sulfonyl]acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-cyclohexylsulfamide obtained in Reference Example 114.

¹H-NMR (300 MHz, CDCl₃) δ:1.10-1.30 (m, 6H), 1.62-1.72 (m, 2H), 1.77-1.88 (m, 2H), 2.44 (s, 3H), 3.08-3.22 (m, 1H), 3.53 (s, 3H), 4.94 (d, J=7.2 Hz, 1H), 5.23 (d, J=15.5 Hz, 1H), 6.79 (d, J=3.0 Hz, 1H), 6.88-6.93 (m, 1H), 7.11 (d, J=3.4 Hz, 1H), 7.21 (dd, J=8.7, 1.9 Hz, 1H), 7.43 (d, J=15.9 Hz, 1H), 7.72 (d, J=1.9 Hz, 1H).

### Example 96

### (3E)-4-[5-(5-chloro-1H-indol-1-yl)-1,3-dimethyl-1H-pyrazol-4-yl]-2-oxo-N-(pentylsulfonyl)but-3-enamide

By a method similar to that in Example 1, the title compound was obtained from (3E)-4-[5-(5-chloro-1H-indol-1-yl)-1,3-dimethyl-1H-pyrazol-4-yl]-2-oxobut-3-enoic acid obtained in Reference Example 94 and pentane-1-sulfonamide.

¹H-NMR (300 MHz, CDCl₃) δ:0.89 (t, J=7.1 Hz, 3H), 1.27-1.45 (m, 4H), 1.75-1.88 (m, 2H), 2.53 (s, 3H), 3.35-3.43 (m, 2H), 3.55 (s, 3H), 6.76-6.83 (m, 2H), 6.88-6.94 (m, 1H), 7.13 (d, J=3.2 Hz, 1H), 7.21 (dd, J=8.7, 1.9 Hz, 1H), 7.64 (d, J=16.2 Hz, 1H), 7.72 (d, J=1.9 Hz, 1H), 9.11 (br s, 1 H).

### Example 97

### 5-{2-[5-(5-chloro-1H-indol-1-yl)-1,3-dimethyl-1H-pyrazol-4-yl]ethyl}-1,3-thiazolidine-2,4-dione

To a solution of ethyl 2-chloro-4-[5-(5-chloro-1H-indol-1-yl)-1,3-dimethyl-1H-pyrazol-4-yl]butanoate obtained in Reference Example 97 (99 mg) in ethanol (2.5 mL) were added thiourea (76 mg) and sodium acetate (82 mg), and the mixture was heated under reflux for 36 hr. 6N Hydrochloric acid (10 mL) was added to this reaction mixture, and the mixture was heated under reflux for 8 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 70:30, v/v) to give the title compound (59 mg, yield 61%) as a colorless amorphous solid.

¹H-NMR (300 MHz, CDCl₃) δ:1.75-1.92 (m, 1H), 2.06-2.21 (m, 1H), 2.25-2.54 (m, 5H), 3.48 (s, 3H), 3.95-4.06 (m, 1H), 6.70 (d, J=3.0 Hz, 1H), 6.93 (d, J=8.7 Hz, 1H), 7.08-7.12 (m, 1H), 7.20 (dd, J=8.7, 1.9 Hz, 1H), 7.67 (d, J=1.9 Hz, 1H), 8.16 (br s, 1H).

### Example 98

### (2E)-3-[5-(5-chloro-1H-indol-1-yl)-1,3-dimethyl-1H-pyrazol-4-yl]-N-(morpholin-4-ylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and morpholine-4-sulfonamide obtained in Reference Example 109.

¹H-NMR (300 MHz, CDCl₃) δ:2.43 (s, 3H), 3.25-3.32 (m, 4H), 3.52 (s, 3H), 3.65-3.73 (m, 4H), 5.35 (d, J=15.8 Hz, 1H), 6.78 (d, J=3.0 Hz, 1H), 6.91 (d, J=8.7 Hz, 1H), 7.11 (d, J=3.2 Hz, 1H), 7.22 (dd, J=8.7, 1.9 Hz, 1H), 7.43 (d, J=15.8 Hz, 1H), 7.67-7.75 (m, 2H).

### Example 99

### (2E)-N-[(butylamino)sulfonyl]-3-[5-(5-chloro-1H-indol-1-yl)-1,3-dimethyl-1H-pyrazol-4-yl]acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-butylsulfamide obtained in Reference Example 111.

¹H-NMR (300 MHz, CDCl₃) δ:0.87 (t, J=7.2 Hz, 3H), 1.23-1.39 (m, 2H), 1.40-1.54 (m, 2H), 2.44 (s, 3H), 2.92 (q, J=6.8 Hz, 2H), 3.53 (s, 3H), 5.06 (t, J=6.1 Hz, 1H), 5.24 (d, J=15.8 Hz, 1H), 6.78 (d, J=2.6 Hz, 1H), 6.91 (d, J=8.7 Hz, 1H), 7.10 (d, J=3.4 Hz, 1H), 7.21 (dd, J=8.8, 2.0 Hz, 1H), 7.42 (d, J=15.8 Hz, 1H), 7.71 (d, J=1.9 Hz, 1H), 7.75 (br s, 1H).

### Example 100

### (2E)-N-[(butylamino)sulfonyl]-3-[1,3-dimethyl-5-(1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-butylsulfamide obtained in Reference Example 111.

¹H-NMR (300 MHz, CDCl₃) δ:0.88 (t, J=7.2 Hz, 3H), 1.24-1.40 (m, 2H), 1.41-1.55 (m, 2H), 2.38 (s, 3H), 2.97 (q, J=6.8 Hz, 2H), 3.59 (s, 3H), 5.12 (t, J=6.2 Hz, 1H), 5.45 (d, J=15.6 Hz, 1H), 6.80 (d, J=3.6 Hz, 1H), 7.18 (d, J=3.6 Hz, 1H), 7.21-7.25 (m, 1H), 7.38 (d, J=15.8 Hz, 1H), 8.06 (dd, J=7.9, 1.5 Hz, 1H), 8.16 (br s, 1H), 8.35 (dd, J=4.7, 1.5 Hz, 1H)

### Example 101

### N-[(butylamino)sulfonyl]-3-[1,3-dimethyl-5-(1H-pyrrolo[2,3-b]pyridin-1-yl)-1H-pyrazol-4-yl]propanamide

By a method similar to that in Example 62, the title compound was obtained from 3-[1,3-dimethyl-5-(1H-pyrrolo[2,3-b]pyridin-1-yl)-1H-pyrazol-4-yl]propanoic acid obtained in Reference Example 45 and N-butylsulfamide obtained in Reference Example 111.

¹H-NMR (300 MHz, CDCl₃) δ:0.92 (t, J=7.2 Hz, 3H), 1.31-1.44 (m, 2H), 1.45-1.55 (m, 2H), 2.01-2.16 (m, 1H), 2.26 (s, 3H), 2.29-2.40 (m, 1H), 2.53-2.68 (m, 2H), 2.72-3.00 (m, 2H), 3.30 (s, 3H), 4.96 (s, 1H), 6.77 (d, J=3.6 Hz, 1H), 7.19 (d, J=3.6 Hz, 1H), 7.29 (dd, J=7.9, 4.8 Hz, 1H), 8.11 (dd, J=7.9, 1.5 Hz, 1H), 8.42 (dd, J=4.8, 1.5 Hz, 1H), 11.87 (s, 1H).

### Example 102

### (2E)-3-[5-(5-chloro-1H-indol-1-yl)-1,3-dimethyl-1H-pyrazol-4-yl]-N-{[(cyclopropylmethyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(cyclopropylmethyl)sulfamide obtained in Reference Example 115.

¹H-NMR (300 MHz, CDCl₃) δ:0.10-0.17 (m, 2H), 0.46-0.54 (m, 2H), 0.84-1.00 (m, 1H), 2.43 (s, 3H), 2.78-2.83 (m, 2H), 3.53 (s, 3H), 5.17-5.22 (m, 2H), 6.78 (dd, J=3.4, 0.8 Hz, 1H), 6.90 (d, J=8.9 Hz, 1H), 7.10 (d, J=3.4 Hz, 1H), 7.21 (dd, J=8.9, 2.0 Hz, 1H), 7.42 (d, J=15.8 Hz, 1H), 7.70 (s, 1H), 7.71 (d, J=2.0 Hz, 1H).

### Example 103

### (2E)-3-[5-(5-chloro-1H-indol-1-yl)-1,3-dimethyl-1H-pyrazol-4-yl]-N-{[(1-ethylpropyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(1-ethylpropyl)sulfamide obtained in Reference Example 113.

¹H-NMR (300 MHz, CDCl₃) δ:0.81-0.86 (m, 6H), 1.30-1.55 (m, 4H), 2.43 (s, 3H), 3.08-3.20 (m, 1H), 3.53 (s, 3H), 4.86 (d, J=7.6 Hz, 1H), 5.25 (d, J=15.9 Hz, 1H), 6.78 (d, J=2.3 Hz, 1H), 6.91 (d, J=8.7 Hz, 1H), 7.11 (d, J=3.4 Hz, 1H), 7.20 (dd, J=8.7, 1.9 Hz, 1H), 7.42 (d, J=15.9 Hz, 1H), 7.71 (d, J=1.9 Hz, 1H), 7.77 (s, 1H).

### Example 104

### ethyl N-[({(2E)-3-[5-(5-chloro-1H-indol-1-yl)-1,3-dimethyl-1H-pyrazol-4-yl]prop-2-enoyl}amino)sulfonyl]glycinate

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and ethyl N-(aminosulfonyl)glycinate obtained in Reference Example 117.

¹H-NMR (300 MHz, CDCl₃) δ:1.23 (t, J=7.2 Hz, 3H), 2.42 (s, 3H), 3.52 (s, 3H), 3.87 (s, 2H), 4.15 (q, J=7.2 Hz, 2H), 5.26 (d, J=15.6 Hz, 1H), 5.59 (s, 1H), 6.78 (d, J=3.4 Hz, 1H), 6.88-6.95 (m, 1H), 7.11 (d, J=3.4 Hz, 1H), 7.21 (dd, J=8.8, 2.0 Hz, 1H), 7.43 (d, J=15.6 Hz, 1H), 7.71 (d, J=2.0 Hz, 1H), 7.80 (br s, 1H).

### Example 105

### (2E)-N-[(butylamino)sulfonyl]-3-[5-(1H-indol-1-yl)-1,3-dimethyl-1H-pyrazol-4-yl]acrylamide

By a method similar to that in Example 62, the title compound was obtained from (2E)-3-[5-(1H-indol-1-yl)-1,3-dimethyl-1H-pyrazol-4-yl]acrylic acid obtained in Reference Example 2 and N-butylsulfamide obtained in Reference Example 111.

¹H-NMR (300 MHz, DMSO-d₆) δ:0.79 (t, J=7.2 Hz, 3H), 1.07-1.30 (m, 2H), 1.30-1.41 (m, 2H), 2.40 (s, 3H), 2.71-2.85 (m, 2H), 3.47 (s, 3H), 6.12 (d, J=15.9 Hz, 1H), 6.85 (d, J=3.0 Hz, 1H), 6.92-7.06 (m, 2H), 7.11-7.25 (m, 2H), 7.52 (t, J=5.7 Hz, 1H), 7.58 (d, J=3.4 Hz, 1H), 7.68-7.81 (m, 1H), 11.31 (s, 1H).

### Example 106

### (2E)-3-[5-(1H-indol-1-yl)-1,3-dimethyl-1H-pyrazol-4-yl]-N-[(propylamino)sulfonyl]acrylamide

By a method similar to that in Example 62, the title compound was obtained from (2E)-3-[5-(1H-indol-1-yl)-1,3-dimethyl-1H-pyrazol-4-yl]acrylic acid obtained in Reference Example 2 and N-propylsulfamide obtained in Reference Example 127.

¹H-NMR (300 MHz, DMSO-d₆) δ:0.78 (t, J=7.4 Hz, 3H), 1.25-1.52 (m, 2H), 2.40 (s, 3H), 2.63-2.86 (m, 2H), 3.47 (s, 3H), 6.12 (d, J=16.3 Hz, 1H), 6.85 (d, J=3.4 Hz, 1H), 6.92-7.07 (m, 2H), 7.13-7.25 (m, 2H), 7.52 (br s, 1H), 7.58 (d, J=3.4 Hz, 1H), 7.65-7.78 (m, 1H), 11.31 (s, 1H).

### Example 107

### (2E)-N-{[(cyclopropylmethyl)amino]sulfonyl}-3-[5-(1H-indol-1-yl)-1,3-dimethyl-1H-pyrazol-4-yl]acrylamide

By a method similar to that in Example 62, the title compound was obtained from (2E)-3-[5-(1H-indol-1-yl)-1,3-dimethyl-1H-pyrazol-4-yl]acrylic acid obtained in Reference Example 2 and N-(cyclopropylmethyl)sulfamide obtained in Reference Example 115.

¹H-NMR (300 MHz, DMSO-d₆) δ:0.05-0.15 (m, 2H), 0.24-0.39 (m, 2H), 0.72-0.94 (m, 1H), 2.40 (s, 3H), 2.71 (t, J=6.4 Hz, 2H), 3.47 (s, 3H), 6.11 (d, J=16.0 Hz, 1H), 6.85 (dd, J=3.3, 0.8 Hz, 1H), 6.99 (d, J=16.0 Hz, 1H), 6.98-7.04 (m, 1 H), 7.15-7.25 (m, 2H), 7.52-7.64 (m, 2H), 7.67-7.82 (m, 1H), 11.32 (s, 1H).

### Example 108

### (2E)-3-[5-(3-chloro-1H-indol-1-yl)-1,3-dimethyl-1H-pyrazol-4-yl]-N-(pentylsulfonyl)acrylamide

By a method similar to that in Example 1, the title compound was obtained from (2E)-3-[5-(3-chloro-1H-indol-1-yl)-1,3-dimethyl-1H-pyrazol-4-yl]acrylic acid obtained in Reference Example 130 and pentane-1-sulfonamide.

¹H-NMR (300 MHz, DMSO-d₆) δ:0.81 (t, J=7.1 Hz, 3H), 1.10-1.42 (m, 4H), 1.46-1.65 (m, 2H), 2.40 (s, 3H), 3.24-3.32 (m, 2 H), 3.50 (s, 3H), 6.07 (d, J=16.0 Hz, 1H), 6.91-7.13 (m, 2H), 7.20-7.41 (m, 2H), 7.60-7.73 (m, 1H), 7.92 (s, 1H), 11.62 (s, 1H).

### Example 109

### (2E)-N-[(butylamino)sulfonyl]-3-[5-(3-chloro-1H-indol-1-yl)-1,3-dimethyl-1H-pyrazol-4-yl]acrylamide

By a method similar to that in Example 62, the title compound was obtained from (2E)-3-[5-(3-chloro-1H-indol-1-yl)-1,3-dimethyl-1H-pyrazol-4-yl]acrylic acid obtained in Reference Example 130 and N-butylsulfamide obtained in Reference Example 111.

¹H-NMR (300 MHz, DMSO-d₆) δ:0.79 (t, J=7.3 Hz, 3H), 1.12-1.28 (m, 2H), 1.28-1.42 (m, 2H), 2.40 (s, 3H), 2.67-2.87 (m, 2H), 3.49 (s, 3H), 6.08 (d, J=16.2 Hz, 1H), 7.00 (d, J=16.0 Hz, 1H), 7.04-7.12 (m, 1H), 7.19-7.41 (m, 2H), 7.54 (t, J=5.6 Hz, 1H), 7.57-7.71 (m, 1H), 7.92 (s, 1H), 11.32 (s, 1H).

### Example 110

### (2E)-3-[5-(6-methoxy-1H-indol-1-yl)-1,3-dimethyl-1H-pyrazol-4-yl]-N-[(4-methylphenyl)sulfonyl]acrylamide

By a method similar to that in Example 1, the title compound was obtained from (2E)-3-[5-(6-methoxy-1H-indol-1-yl)-1,3-dimethyl-1H-pyrazol-4-yl]acrylic acid obtained in Reference Example 60 and 4-methylbenzenesulfonamide.

¹H-NMR (300 MHz, DMSO-d₆) δ:2.36 (s, 3H), 2.37 (s, 3H), 3.46 (s, 3H), 3.66 (s, 3H), 6.05 (d, J=16.0 Hz, 1H), 6.44 (d, J=1.9 Hz, 1H), 6.73 (d, J=2.8 Hz, 1H), 6.83 (dd, J=8.5, 2.3 Hz, 1H), 6.95 (d, J=16.0 Hz, 1H), 7.25-7.39 (m, 3H), 7.58 (d, J=8.5 Hz, 1H), 7.74 (d, J=8.3 Hz, 2H), 12.02 (s, 1H).

### Example 111

### potassium {(2E)-3-[5-(6-methoxy-1H-indol-1-yl)-1,3-dimethyl-1H-pyrazol-4-yl]prop-2-enoyl}[(4-methylphenyl)sulfonyl]azanide

By a method similar to that in Example 7, the title compound was obtained from (2E)-3-[5-(6-methoxy-1H-indol-1-yl)-1,3-dimethyl-1H-pyrazol-4-yl]-N-[(4-methylphenyl)sulfonyl]acrylamide obtained in Example 110.

¹H-NMR (300 MHz, DMSO-d₆) δ:2.27 (s, 3H), 2.32 (s, 3H), 3.41 (s, 3H), 3.67 (s, 3H), 5.56 (d, J=16.0 Hz, 1H), 6.41 (d, J=2.3 Hz, 1H), 6.66-6.76 (m, 2H), 6.81 (dd, J=8.5, 2.3 Hz, 1H), 7.09 (d, J=7.9 Hz, 2H), 7.29 (d, J=3.2 Hz, 1H), 7.48-7.60 (m, 3H).

### Example 112

### (2E)-3-[1,3-dimethyl-5-(3-methyl-1H-pyrrolo[2,3-b]pyridin-1-yl)-1H-pyrazol-4-yl]-N-(pentylsulfonyl)acrylamide

By a method similar to that in Example 1, the title compound was obtained from (2E)-3-[1,3-dimethyl-5-(3-methyl-1H-pyrrolo[2,3-b]pyridin-1-yl)-1H-pyrazol-4-yl]acrylic acid obtained in Reference Example 136 and pentane-1-sulfonamide.

¹H-NMR (300 MHz, DMSO-d₆) δ:0.81 (t, J=7.1 Hz, 3H), 0.97-1.41 (m, 4H), 1.49-1.68 (m, 2H), 2.36 (d, J=1.1 Hz, 3H), 2.38 (s, 3H), 3.27-3.35 (m, 2H), 3.48 (s, 3H), 6.12 (d, J=15.9 Hz, 1H), 7.03 (d, J=15.9 Hz, 1H), 7.27 (dd, J=7.8, 4.7 Hz, 1H), 7.47 (d, J=0.8 Hz, 1H), 8.13 (dd, J=8.0, 1.5 Hz, 1H), 8.25 (dd, J=4.5, 1.5 Hz, 1H), 11.59 (s, 1H).

### Example 113

### (2E)-N-[(butylamino)sulfonyl]-3-[1,3-dimethyl-5-(3-methyl-1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(3-methyl-1H-pyrrolo[2,3-b]pyridin-1-yl)-1H-pyrazol-4-yl]acrylic acid obtained in Reference Example 136 and N-butylsulfamide obtained in Reference Example 111.

¹H-NMR (300 MHz, DMSO-d₆) δ:0.79 (t, J=7.3 Hz, 3H), 1.13-1.28 (m, 2H), 1.29-1.43 (m, 2H), 2.36 (d, J=0.9 Hz, 3H), 2.38 (s, 3H), 2.79 (q, J=6.8 Hz, 2H), 3.48 (s, 3H), 6.13 (d, J=16.0 Hz, 1H), 7.00 (d, J=16.0 Hz, 1H), 7.26 (dd, J=7.9, 4.7 Hz, 1H), 7.47 (d, J=1.1 Hz, 1H), 7.48-7.51 (m, 1H), 8.13 (dd, J=7.8, 1.6 Hz, 1H), 8.25 (dd, J=4.7, 1.3 Hz, 1H), 11.29 (s, 1H).

### Example 114

### (2E)-3-[1,3-dimethyl-5-(3-methyl-1H-pyrrolo[2,3-b]pyridin-1-yl)-1H-pyrazol-4-yl]-N-[(4-methylphenyl)sulfonyl]acrylamide

By a method similar to that in Example 1, the title compound was obtained from (2E)-3-[1,3-dimethyl-5-(3-methyl-1H-pyrrolo[2,3-b]pyridin-1-yl)-1H-pyrazol-4-yl]acrylic acid obtained in Reference Example 136 and 4-methylbenzenesulfonamide.

¹H-NMR (300 MHz, DMSO-d₆) δ:2.34 (d, J=1.1 Hz, 3H), 2.35 (s, 3H), 2.36 (s, 3H), 3.46 (s, 3H), 6.07 (d, J=16.0 Hz, 1H), 6.92 (d, J=16.0 Hz, 1H), 7.25 (dd, J=7.9, 4.7 Hz, 1H), 7.37 (d, J=7.9 Hz, 2H), 7.43 (d, J=1.1 Hz, 1H), 7.74 (d, J=8.3 Hz, 2H), 8.12 (dd, J=7.8, 1.6 Hz, 1H), 8.23 (dd, J=4.7, 1.5 Hz, 1H), 12.00 (s, 1H).

### Example 115

### (2E)-N-{[(cyclopropylmethyl)amino]sulfonyl}-3-[1,3-dimethyl-5-(3-methyl-1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(3-methyl-1H-pyrrolo[2,3-b]pyridin-1-yl)-1H-pyrazol-4-yl]acrylic acid obtained in Reference Example 136 and N-(cyclopropylmethyl)sulfamide obtained in Reference Example 115.

¹H-NMR (300 MHz, DMSO-d₆) δ:0.04-0.16 (m, 2H), 0.26-0.46 (m, 2H), 0.70-0.97 (m, 1H), 2.36 (d, J=1.1 Hz, 3H), 2.37 (s, 3H), 2.72 (t, J=6.4 Hz, 2H), 3.48 (s, 3H), 6.12 (d, J=16.0 Hz, 1H), 6.99 (d, J=16.0 Hz, 1H), 7.26 (dd, J=7.8, 4.8 Hz, 1H), 7.47 (d, J=1.1 Hz, 1H), 7.62 (t, J=6.1 Hz, 1H), 8.13 (dd, J=7.9, 1.5 Hz, 1H), 8.25 (dd, J=4.7, 1.5 Hz, 1H), 11.30 (s, 1H).

### Example 116

### (2E)-3-[1,3-dimethyl-5-(1-methyl-1H-indol-3-yl)-1H-pyrazol-4-yl]-N-(pentylsulfonyl)acrylamide

By a method similar to that in Example 1, the title compound was obtained from (2E)-3-[1,3-dimethyl-5-(1-methyl-1H-indol-3-yl)-1H-pyrazol-4-yl]acrylic acid obtained in Reference Example 140 and pentane-1-sulfonamide.

¹H-NMR (300 MHz, DMSO-d₆) δ:0.82 (t, J=7.0 Hz, 3H), 1.17-1.40 (m, 4H), 1.53-1.66 (m, 2H), 2.38 (s, 3H), 3.26-3.40 (m, 2H), 3.65 (s, 3H), 3.92 (s, 3H), 6.29 (d, J=16.0 Hz, 1H), 7.08-7.17 (m, 1H), 7.25-7.31 (m, 2H), 7.35 (d, J=16.0 Hz, 1H), 7.60 (d, J=8.7 Hz, 1H), 7.68 (s, 1H), 11.49 (s, 1H).

### Example 117

### (2E)-N-[(butylamino)sulfonyl]-3-[1,3-dimethyl-5-(1-methyl-1H-indol-3-yl)-1H-pyrazol-4-yl]acrylamide

By a method similar to that in Example 62, the title compound was obtained from (2E)-3-[1,3-dimethyl-5-(1-methyl-1H-indol-3-yl)-1H-pyrazol-4-yl]acrylic acid obtained in Reference Example 140 and N-butylsulfamide obtained in Reference Example 111.

¹H-NMR (300 MHz, DMSO-d₆) δ:0.81 (t, J=7.3 Hz, 3H), 1.16-1.28 (m, 2H), 1.31-1.44 (m, 2H), 2.37 (s, 3H), 2.75-2.85 (m, 2H), 3.65 (s, 3H), 3.92 (s, 3H), 6.27 (d, J=15.8 Hz, 1H), 7.02-7.17 (m, 1H), 7.23-7.36 (m, 3H), 7.39-7.52 (m, 1H), 7.59 (d, J=8.9 Hz, 1H), 7.67 (s, 1H), 11.20 (s, 1H).

### Example 118

### (2E)-3-[1,3-dimethyl-5-(3-methyl-1H-indazol-1-yl)-1H-pyrazol-4-yl]-N-(pentylsulfonyl)acrylamide

By a method similar to that in Example 1, the title compound was obtained from (2E)-3-[1,3-dimethyl-5-(3-methyl-1H-indazol-1-yl)-1H-pyrazol-4-yl]acrylic acid obtained in Reference Example 143 and pentane-1-sulfonamide.

¹H-NMR (300 MHz, DMSO-d₆) δ:0.87 (t, J=7.0 Hz, 3H), 1.29-1.40 (m, 4H), 1.56-1.78 (m, 2H), 2.45 (s, 3H), 2.63 (s, 3H), 3.24-3.38 (m, 2H), 3.56 (s, 3H), 5.45-5.62 (m, 1H), 7.08 (d, J=8.5 Hz, 1H), 7.28-7.36 (m, 1H), 7.40-7.53 (m, 2H), 7.78 (d, J=8.1 Hz, 1H), 7.86 (br s, 1H).

### Example 119

### potassium {(2E)-3-[1,3-dimethyl-5-(3-methyl-1H-indazol-1-yl)-1H-pyrazol-4-yl]prop-2-enoyl}(pentylsulfonyl)azanide

By a method similar to that in Example 7, the title compound was obtained from (2E)-3-[1,3-dimethyl-5-(3-methyl-1H-indazol-1-yl)-1H-pyrazol-4-yl]-N-(pentylsulfonyl)acrylamide obtained in Example 118.

¹H-NMR (300 MHz, DMSO-d₆) δ:0.80 (t, J=6.6 Hz, 3H), 1.14-1.24 (m, 4H), 1.32-1.50 (m, 2H), 2.35 (s, 3H), 2.62 (s, 3H), 2.85 (t, J=7.8 Hz, 2H), 3.44 (s, 3H), 5.64 (d, J=16.2 Hz, 1H), 6.75 (d, J=16.2 Hz, 1H), 7.14 (d, J=8.5 Hz, 1H), 7.20-7.35 (m, 1H), 7.42-7.52 (m, 1H), 7.89 (d, J=8.1 Hz, 1H).

### Example 120

### (2E)-N-[(butylamino)sulfonyl]-3-[1,3-dimethyl-5-(3-methyl-1H-indazol-1-yl)-1H-pyrazol-4-yl]acrylamide

By a method similar to that in Example 62, the title compound was obtained from (2E)-3-[1,3-dimethyl-5-(3-methyl-1H-indazol-1-yl)-1H-pyrazol-4-yl]acrylic acid obtained in Reference Example 143 and N-butylsulfamide obtained in Reference Example 111.

¹H-NMR (300 MHz, DMSO-d₆) δ:0.79 (t, J=7.3 Hz, 3H), 1.11-1.27 (m, 2H), 1.27-1.43 (m, 2H), 2.40 (s, 3H), 2.63 (s, 3H), 2.68-2.88 (m, 2H), 3.52 (s, 3H), 6.09 (d, J=16.4 Hz, 1H), 7.04 (d, J=16.0 Hz, 1H), 7.20 (d, J=8.3 Hz, 1H), 7.25-7.37 (m, 1H), 7.42-7.59 (m, 2H), 7.92 (d, J=7.9 Hz, 1H), 11.28 (s, 1H).

### Example 121

### (2E)-N-{[(cyclopropylmethyl)amino]sulfonyl}-3-[1,3-dimethyl-5-(3-methyl-1H-indazol-1-yl)-1H-pyrazol-4-yl]acrylamide

By a method similar to that in Example 62, the title compound was obtained from (2E)-3-[1,3-dimethyl-5-(3-methyl-1H-indazol-1-yl)-1H-pyrazol-4-yl]acrylic acid obtained in Reference Example 143 and N-(cyclopropylmethyl)sulfamide obtained in Reference Example 115.

¹H-NMR (300 MHz, DMSO-d₆) δ:0.04-0.16 (m, 2H), 0.25-0.44 (m, 2H), 0.71-0.93 (m, 1H), 2.39 (s, 3H), 2.62 (s, 3H), 2.67-2.74 (m, 2H), 3.52 (s, 3H), 6.06 (d, J=16.6 Hz, 1H), 7.02 (d, J=16.2 Hz, 1H), 7.19 (d, J=8.5 Hz, 1H), 7.32 (t, J=7.1 Hz, 1H), 7.44-7.52 (m, 1H), 7.53-7.68 (m, 1H), 7.92 (d, J=7.7 Hz, 1H), 11.30 (s, 1H).

### Example 122

### (2E)-3-[5-(6-methoxy-1H-pyrrolo[2,3-b]pyridin-1-yl)-1,3-dimethyl-1H-pyrazol-4-yl]-N-(pentylsulfonyl)acrylamide

By a method similar to that in Example 1, the title compound was obtained from (2E)-3-[5-(6-methoxy-1H-pyrrolo[2,3-b]pyridin-1-yl)-1,3-dimethyl-1H-pyrazol-4-yl]acrylic acid obtained in Reference Example 145 and pentane-1-sulfonamide.

¹H-NMR (300 MHz, CDCl₃) δ:0.88 (t, J=6.8 Hz, 3H), 1.21-1.47 (m, 4H), 1.69-1.86 (m, 2H), 2.45 (s, 3H), 3.28-3.48 (m, 2H), 3.66 (s, 3H), 3.83 (s, 3H), 5.51 (d, J=15.8 Hz, 1H), 6.65-6.75 (m, 2H), 6.95 (d, J=3.2 Hz, 1H), 7.50 (d, J=15.8 Hz, 1H), 7.89 (d, J=8.7 Hz, 1H).

### Example 123

### (2E)-N-[(butylamino)sulfonyl]-3-[5-(6-methoxy-1H-pyrrolo[2,3-b]pyridin-1-yl)-1,3-dimethyl-1H-pyrazol-4-yl]acrylamide

By a method similar to that in Example 62, the title compound was obtained from (2E)-3-[5-(6-methoxy-1H-pyrrolo[2,3-b]pyridin-1-yl)-1,3-dimethyl-1H-pyrazol-4-yl]acrylic acid obtained in Reference Example 145 and N-butylsulfamide obtained in Reference Example 111.

¹H-NMR (300 MHz, DMSO-d₆) δ:0.80 (t, J=7.3 Hz, 3H), 1.14-1.30 (m, 2H), 1.31-1.47 (m, 2H), 2.38 (s, 3H), 2.75-2.85 (m, 2H), 3.57 (s, 3H), 3.73 (s, 3H), 6.16 (d, J=16.0 Hz, 1H), 6.72 (d, J=8.5 Hz, 1H), 6.77 (d, J=3.6 Hz, 1H), 7.11 (d, J=16.0 Hz, 1H), 7.44 (d, J=3.6 Hz, 1H), 7.50 (t, J=5.5 Hz, 1H), 8.04 (d, J=8.5 Hz, 1H), 11.34 (br s, 1H).

### Example 124

### (2E)-N-[(butylamino)sulfonyl]-3-[3-cyclopropyl-5-(5-fluoro-1H-indol-1-yl)-1-methyl-1H-pyrazol-4-yl]acrylamide

By a method similar to that in Example 62, the title compound was obtained from (2E)-3-[3-cyclopropyl-5-(5-fluoro-1H-indol-1-yl)-1-methyl-1H-pyrazol-4-yl]acrylic acid obtained in Reference Example 155 and N-butylsulfamide obtained in Reference Example 111.

1H NMR (300 MHz, CDCl₃) δ:0.88 (t, J=7.3 Hz, 3H), 0.92-1.08 (m, 4H), 1.25-1.38 (m, 2H), 1.41-1.53 (m, 2H), 1.91-2.02 (m, 1H), 2.92 (q, J=6.6 Hz, 2H), 3.50 (s, 3H), 5.02-5.11 (m, 1H), 5.40 (d, J=15.6 Hz, 1H), 6.79 (d, J=3.4 Hz, 1H), 6.87-6.94 (m, 1H), 6.95-7.04 (m, 1H), 7.11 (d, J=3.2 Hz, 1H), 7.38 (dd, J=9.0, 2.4 Hz, 1H), 7.55 (d, J=15.8 Hz, 1H).

### Example 125

### (2E)-N-{[(cyclopropylmethyl)amino]sulfonyl}-3-[3-cyclopropyl-1-methyl-5-(1H-pyrrolo[2,3-b]pyridin-1-yl)-1H-pyrazol-4-yl]acrylamide

By a method similar to that in Example 62, the title compound was obtained from (2E)-3-[3-cyclopropyl-1-methyl-5-(1H-pyrrolo[2,3-b]pyridin-1-yl)-1H-pyrazol-4-yl]acrylic acid obtained in Reference Example 218 and N-(cyclopropylmethyl)sulfamide obtained in Reference Example 115.

¹H-NMR (300 MHz, CDCl₃) δ:0.16-0.21 (m, 2H), 0.48-0.54 (m, 3H), 0.77-0.81 (m, 2H), 0.90-1.02 (m, 2H), 1.63-1.72 (m, 1H), 2.87-2.93 (m, 2H), 3.54 (s, 3H), 5.27 (t, J=6.0 Hz, 1H), 5.75 (d, J=15.6 Hz, 1H), 6.78 (d, J=3.6 Hz, 1H), 7.18-7.24 (m, 2H), 7.34 (d, J=15.6 Hz, 1H), 8.03-8.07 (m, 1H), 8.29 (dd, J=4.8, 1.5 Hz, 1H).

### Example 126

### (2E)-N-{[(cyclopropylmethyl)amino]sulfonyl}-3-[1-methyl-5-(1H-pyrrolo[2,3-b]pyridin-1-yl)-3-(trifluoromethyl)-1H-pyrazol-4-yl]acrylamide

By a method similar to that in Example 62, the title compound was obtained from (2E)-3-[1-methyl-5-(1H-pyrrolo[2,3-b]pyridin-1-yl)-3-(trifluoromethyl)-1H-pyrazol-4-yl]acrylic acid obtained in Reference Example 230 and N-(cyclopropylmethyl)sulfamide obtained in Reference Example 115.

¹H-NMR (300 MHz, DMSO-d₆) δ:0.07-0.09 (m, 2H), 0.27-0.33 (m, 2H), 0.75-0.87 (m, 1H), 2.70 (t, J=6.6 Hz, 2H), 3.63 (s, 3H), 5.88 (d, J=15.9 Hz, 1H), 6.93 (d, J=3.9 Hz, 1H), 7.15 (d, J=15.9 Hz, 1H), 7.30 (dd, J=7.8, 4.5 Hz, 1H), 7.69-7.71 (m, 1H), 7.77 (d, J=3.6 Hz, 1H), 8.19 (dd, J=7.8, 1.5 Hz, 1H), 8.28 (dd, J=4.8, 1.5 Hz, 1H).

### Example 127

### (2E)-N-[(butylamino)sulfonyl]-3-[1-methyl-5-(1H-pyrrolo[2,3-b]pyridin-1-yl)-3-(trifluoromethyl)-1H-pyrazol-4-yl]acrylamide

By a method similar to that in Example 62, the title compound was obtained from (2E)-3-[1-methyl-5-(1H-pyrrolo[2,3-b]pyridin-1-yl)-3-(trifluoromethyl)-1H-pyrazol-4-yl]acrylic acid obtained in Reference Example 230 and N-butylsulfamide obtained in Reference Example 111.

¹H-NMR (300 MHz, DMSO-d₆) δ:0.78 (t, J=7.2 Hz, 3H), 1.14-1.38 (m, 4H), 2.74-2.80 (m, 2H), 3.63 (s, 3H), 5.90 (d, J=16.2 Hz, 1H), 6.93 (d, J=3.3 Hz, 1H), 7.16 (d, J=15.3 Hz, 1H), 7.31 (dd, J=8.1, 4.8 Hz, 1H), 7.58-7.60 (m, 1H), 7.77 (d, J=3.6 Hz, 1H), 8.19 (dd, J=7.8, 1.5 Hz, 1H), 8.29 (dd, J=4.8, 1.5 Hz, 1H).

### Example 128

### (2E)-N-{[(3-methylbutyl)amino]sulfonyl}-3-[1-methyl-5-(1H-pyrrolo[2,3-b]pyridin-1-yl)-3-(trifluoromethyl)-1H-pyrazol-4-yl]acrylamide

By a method similar to that in Example 62, the title compound was obtained from (2E)-3-[1-methyl-5-(1H-pyrrolo[2,3-b]pyridin-1-yl)-3-(trifluoromethyl)-1H-pyrazol-4-yl]acrylic acid obtained in Reference Example 230 and N-(3-methylbutyl)sulfamide obtained in Reference Example 125.

¹H-NMR (300 MHz, DMSO-d₆) δ:0.76 (d, J=6.6 Hz, 6H), 1.20-1.28 (m, 2H), 1.46-1.58 (m, 1H), 2.7.6-2.82 (m, 2H), 3.63 (s, 3H), 5.90 (d, J=15.9 Hz, 1H), 6.93 (d, J=3.6 Hz, 1H), 7.16 (d, J=15.9 Hz, 1H), 7.31 (dd, J=7.8, 4.8 Hz, 1H), 7.60 (br s, 1H), 7.78 (d, J=3.9 Hz, 1H), 8.19 (dd, J=7.8, 1.8 Hz, 1H), 8.29 (dd, J=4.8, 1.8 Hz, 1H).

### Example 129

### (2E)-3-[1-methyl-5-(1H-pyrrolo[2,3-b]pyridin-1-yl)-3-(trifluoromethyl)-1H-pyrazol-4-yl]-N-[(propylamino)sulfonyl]acrylamide

By a method similar to that in Example 62, the title compound was obtained from (2E)-3-[1-methyl-5-(1H-pyrrolo[2,3-b]pyridin-1-yl)-3-(trifluoromethyl)-1H-pyrazol-4-yl]acrylic acid obtained in Reference Example 230 and N-propylsulfamide obtained in Reference Example 127.

¹H-NMR (300 MHz, DMSO-d₆) δ:0.77 (t, J=7.2 Hz, 3H), 1.31-1.43 (m, 2H), 2.70-2.77 (m, 2H), 3.63 (s, 3H), 5.90 (d, J=15.9 Hz, 1H), 6.93 (d, J=3.9 Hz, 1H), 7.16 (d, J=15.9 Hz, 1H), 7.31 (dd, J=8.1, 5.1 Hz, 1H), 7.63 (t, J=5.7 Hz, 1H), 7.78 (d, J=3.9 Hz, 1H), 8.19 (dd, J=8.1, 1.8 Hz, 1H), 8.29 (dd, J=5.1, 1.8 Hz, 1H).

### Example 130

### (2E)-3-[5-(5-chloro-1H-indol-1-yl)-1,3-dimethyl-1H-pyrazol-4-yl]-N-[(propylamino)sulfonyl]acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-propylsulfamide obtained in Reference Example 127.

¹H-NMR (300 MHz, CDCl₃) δ:0.90 (t, J=7.2 Hz, 3H), 1.46-1.58 (m, 2H), 2.43 (s, 3H), 2.85-2.92 (m, 2H), 3.52 (s, 3H), 5.10 (t, J=6.3 Hz, 1H), 5.25 (d, J=15.9 Hz, 1H), 6.77 (d, J=3.3 Hz, 1H), 6.89 (d, J=8.7 Hz, 1H), 7.09 (d, J=3.3 Hz, 1H), 7.20 (dd, J=8.7, 1.8 Hz, 1H), 7.41 (d, J=15.9 Hz, 1H), 7.70 (d, J=1.8 Hz, 1H), 7.81 (br s, 1H).

### Example 131

### (2E)-3-[3-cyclopropyl-1-methyl-5-(1H-pyrrolo[2,3-b]pyridin-1-yl)-1H-pyrazol-4-yl]-N-[(propylamino)sulfonyl]acrylamide

By a method similar to that in Example 62, the title compound was obtained from (2E)-3-[3-cyclopropyl-1-methyl-5-(1H-pyrrolo[2,3-b]pyridin-1-yl)-1H-pyrazol-4-yl]acrylic acid obtained in Reference Example 218 and N-propylsulfamide obtained in Reference Example 127.

¹H-NMR (300 MHz, CDCl₃) δ:0.60-0.67 (m, 1H), 0.81-0.95 (m, 6H), 1.49-1.62 (m, 2H), 1.68-1.78 (m, 1H), 2.93-3.02 (m, 2H), 3.55 (s, 3H), 5.16 (t, J=6.0 Hz, 1H), 5.75 (d, J=15.6 Hz, 1H), 6.78 (d, J=3.9 Hz, 1H), 6.99-7.23 (m, 2H), 7.38 (d, J=15.6 Hz, 1H), 8.06 (d, J=7.8 Hz, 1H), 8.31 (d, J=4.8 Hz, 1H), 9.14 (br s, 1H).

### Example 132

### (2E)-3-[3-cyclopropyl-1-methyl-5-(1H-pyrrolo[2,3-b]pyridin-1-yl)-1H-pyrazol-4-yl]-N-{[(3-methylbutyl)amino]sulfonyl}acrylamide

By a method similar to that in Example 62, the title compound was obtained from (2E)-3-[3-cyclopropyl-1-methyl-5-(1H-pyrrolo[2,3-b]pyridin-1-yl)-1H-pyrazol-4-yl]acrylic acid obtained in Reference Example 218 and N-(3-methylbutyl)sulfamide obtained in Reference Example 125.

¹H-NMR (300 MHz, CDCl₃) δ:0.50-0.57 (m, 1H), 0.78-0.94 (m, 9H), 1.40 (q, J=7.2 Hz, 2H), 1.60-1.69 (m, 2H), 3.02-3.05 (m, 2H), 3.55 (s, 3H), 5.14 (t, J=6.0 Hz, 1H), 5.79 (d, J=15.6 Hz, 1H), 6.78 (d, J=3.6 Hz, 1H), 7.18-7.23 (m, 2H), 7.34 (d, J=15.6 Hz, 1H), 8.06 (d, J=7.8 Hz, 1H), 8.29 (d, J=4.8 Hz, 1H), 9.56 (br s, 1H).

### Example 133

### (2E)-N-{[(cyclopropylmethyl)amino]sulfonyl}-3-[3-(difluoromethyl)-1-methyl-5-(1H-pyrrolo[2,3-b]pyridin-1-yl)-1H-pyrazol-4-yl]acrylamide

By a method similar to that in Example 62, the title compound was obtained from (2E)-3-[3-(difluoromethyl)-1-methyl-5-(1H-pyrrolo[2,3-b]pyridin-1-yl)-1H-pyrazol-4-yl]acrylic acid obtained in Reference Example 265 and N-(cyclopropylmethyl)sulfamide obtained in Reference Example 115.

¹H-NMR (300 MHz, DMSO-d₆) δ:0.06-0.10 (m, 2H), 0.28-0.35 (m, 2H), 0.78-0.85 (m, 1H), 2.70 (t, J=6.3 Hz, 2H), 3.59 (s, 3H), 5.96 (d, J=15.9 Hz, 1H), 6.91 (d, J=3.6 Hz, 1H), 7.01-7.36 (m, 3H), 7.67 (t, J=6.3 Hz, 1H), 7.77 (d, J=3.6 Hz, 1H), 8.17-8.20 (m, 1H), 8.27 (dd, J=4.8, 1.5 Hz, 1H).

### Example 134

### (2E)-3-[3-cyclopropyl-1-methyl-5-(1H-pyrrolo[2,3-b]pyridin-1-yl)-1H-pyrazol-4-yl]-N-(pentylsulfonyl)acrylamide

By a method similar to that in Example 1, the title compound was obtained from (2E)-3-[3-cyclopropyl-1-methyl-5-(1H-pyrrolo[2,3-b]pyridin-1-yl)-1H-pyrazol-4-yl]acrylic acid obtained in Reference Example 218 and pentane-1-sulfonamide.

¹H-NMR (300 MHz, CDCl₃) δ:0.44-0.50 (m, 1H), 0.73-0.76 (m, 2H), 0.87-0.94 (m, 4H), 1.28-1.42 (m, 4H), 1.58-1.65 (m, 1H), 1.76-1.86 (m, 2H), 3.43 (q, J=7.8 Hz, 2H), 3.52 (s, 3H), 5.83 (d, J=15.6 Hz, 1H), 6.76 (d, J=3.6 Hz, 1H), 7.17-7.25 (m, 2H), 7.34 (d, J=15.6 Hz, 1H), 8.04 (dd, J=7.8, 1.5 Hz, 1H), 8.29 (dd, J=4.8, 1.5 Hz, 1H), 9.87 (br s, 1H).

### Example 135

### (2E)-3-[1-methyl-5-(1H-pyrrolo[2,3-b]pyridin-1-yl)-3-(trifluoromethyl)-1H-pyrazol-4-yl]-N-[(pentylamino)sulfonyl]acrylamide

By a method similar to that in Example 62, the title compound was obtained from (2E)-3-[1-methyl-5-(1H-pyrrolo[2,3-b]pyridin-1-yl)-3-(trifluoromethyl)-1H-pyrazol-4-yl]acrylic acid obtained in Reference Example 230 and N-pentylsulfamide obtained in Reference Example 287.

¹H-NMR (300 MHz, DMSO-d₆) δ:0.70-0.85 (m, 3H), 1.05-1.24 (m, 4H), 1.25-1.44 (m, 2H), 2.68-2.83 (m, 2H), 3.63 (s, 3H), 5.90 (d, J=16.0 Hz, 1H), 6.94 (d, J=3.8 Hz, 1H), 7.17 (d, J=16.0 Hz, 1H), 7.31 (dd, J=7.8, 4.8 Hz, 1H), 7.60 (br s, 1H), 7.78 (d, J=3.6 Hz, 1H), 8.20 (dd, J=7.8, 1.6 Hz, 1H), 8.30 (dd, J=4.7, 1.5 Hz, 1H), 11.53 (s, 1H).

### Example 136

### (2E)-3-[1,3-dimethyl-5-(1H-pyrrolo[2,3-b]pyridin-1-yl)-1H-pyrazol-4-yl]-N-[(pentylamino)sulfonyl]acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-pentylsulfamide obtained in Reference Example 287.

¹H-NMR (300 MHz, DMSO-d₆) δ:0.70-0.83 (m, 3H), 1.09-1.26 (m, 4H), 1.28-1.44 (m, 2H), 2.39 (s, 3H), 2.79 (q, J=6.8 Hz, 2H), 3.49 (s, 3H), 6.11 (d, J=16.2 Hz, 1H), 6.88 (d, J=3.6 Hz, 1H), 7.00 (d, J=16.0 Hz, 1H), 7.27 (dd, J=7.9, 4.7 Hz, 1H), 7.51 (br s, 1H), 7.71 (d, J=3.8 Hz, 1H), 8.16 (dd, J=7.8, 1.6 Hz, 1H), 8.27 (dd, J=4.7, 1.5 Hz, 1H), 11.29 (s, 1H).

### Example 137

### (2E)-3-[5-(5-chloro-1H-indol-1-yl)-1,3-dimethyl-1H-pyrazol-4-yl]-N-[(ethylamino)sulfonyl]acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-ethylsulfamide obtained in Reference Example 152.

¹H-NMR (300 MHz, CDCl₃) δ:1.13 (t, J=7.3 Hz, 3H), 2.42 (s, 3H), 2.92-3.04 (m, 2H), 3.52 (s, 3H), 5.13 (br s, 1H), 5.29 (d, J=15.8 Hz, 1H), 6.77 (d, J=3.2 Hz, 1H), 6.90 (d, J=8.7 Hz, 1H), 7.10 (d, J=3.2 Hz, 1H), 7.20 (dd, J=8.7, 1.9 Hz, 1H), 7.41 (d, J=15.8 Hz, 1H), 7.70 (d, J=1.7 Hz, 1H), 8.04 (br s, 1H).

### Example 138

### (2E)-3-[5-(5-chloro-1H-indol-1-yl)-1,3-dimethyl-1H-pyrazol-4-yl]-N-(1,4-dioxa-8-azaspiro[4.5]dec-8-ylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and 1,4-dioxa-8-azaspiro[4.5]decane-8-sulfonamide obtained in Reference Example 120.

¹H-NMR (300 MHz, CDCl₃) δ:1.66-1.80 (m, 4H), 2.42 (s, 3H), 3.33-3.55 (m, 7H), 3.93 (s, 4H), 5.43 (d, J=15.9 Hz, 1H), 6.73-6.78 (m, 1H), 6.91 (d, J=8.7 Hz, 1H), 7.11 (d, J=3.0 Hz, 1H), 7.21 (d, J=8.3 Hz, 1H), 7.40 (d, J=15.5 Hz, 1H), 7.68 (s, 1H), 8.14 (br s, 1H).

### Example 139

### (2E)-3-[5-(5-chloro-1H-indol-1-yl)-1,3-dimethyl-1H-pyrazol-4-yl]-N-{[(3-methylbutyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(3-methylbutyl)sulfamide obtained in Reference Example 125.

¹H-NMR (300 MHz, CDCl₃) δ:0.85 (d, J=6.6 Hz, 6H), 1.37 (q, J=7.0 Hz, 2H), 1.52-1.68 (m, 1H), 2.42 (s, 3H), 2.92 (q, J=7.1 Hz, 2H), 3.52 (s, 3H), 5.15 (t, J=6.1 Hz, 1H), 5.33 (d, J=15.8 Hz, 1H), 6.77 (d, J=3.2 Hz, 1H), 6.90 (d, J=8.7 Hz, 1H), 7.10 (d, J=3.2 Hz, 1H), 7.19 (dd, J=8.7, 1.9 Hz, 1H), 7.40 (d, J=15.8 Hz, 1H), 7.69 (d, J=1.7 Hz, 1H), 8.19 (br s, 1H).

### Example 140

### (2E)-3-[5-(5-chloro-1H-indol-1-yl)-1,3-dimethyl-1H-pyrazol-4-yl]-N-{[(cyclohexylmethyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(cyclohexylmethyl)sulfamide obtained in Reference Example 123.

¹H-NMR (300 MHz, CDCl₃) δ:0.80-0.94 (m, 2H), 1.09-1.19 (m, 2H), 1.37-1.49 (m, 1H), 1.61-1.74 (m, 6H), 2.42 (s, 3H), 2.74 (t, J=6.6 Hz, 2H), 3.51 (s, 3H), 5.21 (t, J=6.3 Hz, 1H), 5.32 (d, J=15.8 Hz, 1H), 6.77 (d, J=3.2 Hz, 1H), 6.90 (d, J=8.7 Hz, 1H), 7.10 (d, J=3.2 Hz, 1H), 7.19 (dd, J=8.7, 1.9 Hz, 1H), 7.69 (d, J=1.7 Hz, 1H), 8.13 (br s, 1H).

### Example 141

### (2E)-3-[5-(5-chloro-1H-indol-1-yl)-1,3-dimethyl-1H-pyrazol-4-yl]-N-{[(3-isopropoxypropyl)amino]sulfonyl}acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-(3-isopropoxypropyl)sulfamide obtained in Reference Example 121.

¹H-NMR (300 MHz, CDCl₃) δ:1.12 (d, J=6.0 Hz, 6H), 1.70-1.80 (m, 2H), 2.45 (s, 3H), 3.02-3.12 (m, 2H), 3.42-3.60 (m, 6H), 5.31 (d, J=15.8 Hz, 1H), 5.72 (br s, 1H), 6.79 (d, J=2.8 Hz, 1H), 6.91 (d, J=8.7 Hz, 1H), 7.10 (d, J=3.2 Hz, 1H), 7.22 (dd, J=8.7, 1.9 Hz, 1H), 7.41 (d, J=15.8 Hz, 1H), 7.71 (d, J=1.7 Hz, 1H), 7.90 (br s, 1H).

### Example 142

### (2E)-3-[5-(5-chloro-1H-indol-1-yl)-1,3-dimethyl-1H-pyrazol-4-yl]-N-[(4-oxopiperidin-1-yl)sulfonyl]acrylamide

To a solution of (2E)-3-[5-(5-chloro-1H-indol-1-yl)-1,3-dimethyl-1H-pyrazol-4-yl]-N-(1,4-dioxa-8-azaspiro[4.5]dec-8-ylsulfonyl)acrylamide obtained in Example 138 (2.53 g) in tetrahydrofuran (10 mL) was added 1N hydrochloric acid (10 mL), and the mixture was stirred with heating at 70° C. for 3 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 35:65, v/v) to give the title compound (2.23 g, yield 96%) as a colorless amorphous solid.

¹H-NMR (300 MHz, CDCl₃) δ:2.43 (s, 3H), 2.52 (t, J=6.2 Hz, 4H), 3.52 (s, 3H), 3.66 (t, J=6.1 Hz, 4H), 5.28 (d, J=15.6 Hz, 1H), 6.78 (d, J=3.2 Hz, 1H), 6.90 (d, J=8.7 Hz, 1H), 7.10 (d, J=3.4 Hz, 1H), 7.22 (dd, J=8.7, 1.9 Hz, 1H), 7.43 (d, J=15.6 Hz, 1H), 7.71 (d, J=1.9 Hz, 1H), 7.81 (br s, 1H).

### Example 143

### ethyl N-[({(2E)-3-[5-(5-chloro-1H-indol-1-yl)-1,3-dimethyl-1H-pyrazol-4-yl]prop-2-enoyl}amino)sulfonyl]-β-alaninate

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and ethyl N-(aminosulfonyl)-β-alaninate obtained in Reference Example 260.

¹H-NMR (300 MHz, CDCl₃) δ:1.17-1.25 (m, 3H), 2.40 (s, 3H), 2.53 (t, J=6.4 Hz, 2H), 3.23 (q, J=5.7 Hz, 2H), 3.50 (s, 3H), 4.10 (q, J=7.2 Hz, 2H), 5.81 (br s, 1H), 6.75 (d, J=3.4 Hz, 1H), 6.89 (d, J=8.7 Hz, 1H), 7.11 (d, J=3.0 Hz, 1H), 7.18 (dd, J=8.7, 2.3 Hz, 1H), 7.36-7.43 (m, 1H), 7.67 (d, J=1.9 Hz, 1H), 8.60 (br s, 1H).

### Example 144

### (2E)-3-[1,3-dimethyl-5-(1H-pyrrolo[2,3-b]pyridin-1-yl)-1H-pyrazol-4-yl]-N-{[(1-propylbutyl)amino]sulfonyl}acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-(1-propylbutyl)sulfamide obtained in Reference Example 112.

¹H-NMR (300 MHz, CDCl₃) δ:0.82 (t, J=6.8 Hz, 6H), 1.19-1.47 (m, 8H), 2.37 (s, 3H), 3.24-3.37 (m, 1H), 3.59 (s, 3H), 4.95 (d, J=7.6 Hz, 1H), 5.51 (d, J=15.9 Hz, 1H), 6.79 (d, J=3.4 Hz, 1H), 7.18 (d, J=3.8 Hz, 1H), 7.23 (dd, J=7.8, 4.7 Hz, 1H), 7.36 (d, J=15.9 Hz, 1H), 8.05 (dd, J=8.0, 1.5 Hz, 1H), 8.33 (dd, J=4.9, 1.5 Hz, 1H).

### Example 145

### (2E)-N-[(cyclohexylamino)sulfonyl]-3-[1,3-dimethyl-5-(1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-cyclohexylsulfamide obtained in Reference Example 114.

¹H-NMR (300 MHz, CDCl₃) δ:1.17-1.32 (m, 5H), 1.49-1.57 (m, 1H), 1.63-1.73 (m, 2H), 1.79-1.91 (m, 2H), 2.37 (s, 3H), 3.15-3.26 (m, 1H), 3.59 (s, 3H), 5.05 (d, J=6.8 Hz, 1H), 5.47 (d, J=15.9 Hz, 1H), 6.80 (d, J=3.8 Hz, 1H), 7.19 (d, J=3.8 Hz, 1H), 7.21-7.25 (m, 1H), 7.37 (d, J=15.5 Hz, 1H), 8.06 (dd, J=7.6, 1.5 Hz, 1H), 8.32-8.36 (m, 1H).

### Example 146

### (2E)-N-{[(cyclohexylmethyl)amino]sulfonyl}-3-[1,3-dimethyl-5-(1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-(cyclohexylmethyl)sulfamide obtained in Reference Example 123.

¹H-NMR (300 MHz, CDCl₃) δ:0.82-0.96 (m, 2H), 1.08-1.29 (m, 4H), 1.65-1.77 (m, 5H), 2.31 (s, 3H), 2.83 (t, J=6.4 Hz, 2H), 3.56 (s, 3H), 5.26 (t, J=6.2 Hz, 1H), 5.49 (d, J=15.5 Hz, 1H), 6.78 (d, J=3.8 Hz, 1H), 7.18 (d, J=3.8 Hz, 1H), 7.23 (dd, J=8.0, 4.5 Hz, 1H), 7.33 (d, J=15.9 Hz, 1H), 8.03-8.07 (m, 1H), 8.30-8.34 (m, 1H), 8.82 (br s, 1H).

### Example 147

### (2E)-3-[1,3-dimethyl-5-(1H-pyrrolo[2,3-b]pyridin-1-yl)-1H-pyrazol-4-yl]-N-[(propylamino)sulfonyl]acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-propylsulfamide obtained in Reference Example 127.

¹H-NMR (300 MHz, CDCl₃) δ:0.91 (t, J=7.4 Hz, 3H), 1.47-1.58 (m, 2H), 2.31 (s, 3H), 2.97 (q, J=6.3 Hz, 2H), 3.56 (s, 3H), 5.23 (br s, 1H), 5.50 (d, J=15.9 Hz, 1H), 6.78 (d, J=3.8 Hz, 1H), 7.18 (d, J=3.4 Hz, 1H), 7.21-7.27 (m, 1H), 7.33 (d, J=15.5 Hz, 1H), 8.05 (d, J=7.6 Hz, 1H), 8.32 (d, J=4.2 Hz, 1H), 8.86 (br s, 1H).

### Example 148

### (2E)-3-[5-(3-chloro-1H-pyrrolo[2,3-b]pyridin-1-yl)-1,3-dimethyl-1H-pyrazol-4-yl]-N-{[(cyclopropylmethyl)amino]sulfonyl}acrylamide

By a method similar to that in Example 62, the title compound was obtained from (2E)-3-[5-(3-chloro-1H-pyrrolo[2,3-b]pyridin-1-yl)-1,3-dimethyl-1H-pyrazol-4-yl]acrylic acid obtained in Reference Example 133 and N-(cyclopropylmethyl)sulfamide obtained in Reference Example 115.

¹H-NMR (300 MHz, DMSO-d₆) δ:0.10 (d, J=4.5 Hz, 2H), 0.33 (d, J=7.6 Hz, 2H), 0.84 (t, J=7.4 Hz, 1H), 2.38 (s, 3H), 2.72 (t, J=6.1 Hz, 2H), 3.50 (s, 3H), 6.05 (d, J=15.9 Hz, 1H), 7.01 (d, J=16.3 Hz, 1H), 7.40 (dd, J=7.8, 4.7 Hz, 1H), 7.63 (br s, 1H), 8.06 (s, 1H), 8.16 (d, J=7.6 Hz, 1H), 8.37 (d, J=4.5 Hz, 1H), 11.31 (br s, 1H).

### Example 149

### (2E)-3-[1,3-dimethyl-5-(1H-pyrrolo[2,3-b]pyridin-1-yl)-1H-pyrazol-4-yl]-N-{[(3-methylbutyl)amino]sulfonyl}acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-(3-methylbutyl)sulfamide obtained in Reference Example 125.

¹H-NMR (300 MHz, CDCl₃) δ:0.87 (d, J=6.8 Hz, 6H), 1.40 (q, J=6.9 Hz, 2H), 1.56-1.70 (m, 1H), 2.27 (s, 3H), 2.98-3.09 (m, 2H), 3.55 (s, 3H), 5.22 (t, J=6.1 Hz, 1H), 5.53 (d, J=15.5 Hz, 1H), 6.78 (d, J=3.4 Hz, 1H), 7.18 (d, J=3.4 Hz, 1H), 7.23 (dd, J=8.0, 4.5 Hz, 1H), 7.30 (d, J=15.9 Hz, 1H), 8.05 (d, J=8.0 Hz, 1H), 8.30 (d, J=3.4 Hz, 1H), 9.27 (br s, 1H).

### Example 150

### (2E)-N-[(butylamino)sulfonyl]-3-[5-(5-cyano-1H-indol-1-yl)-1,3-dimethyl-1H-pyrazol-4-yl]acrylamide

By a method similar to that in Example 62, the title compound was obtained from (2E)-3-[5-(5-cyano-1H-indol-1-yl)-1,3-dimethyl-1H-pyrazol-4-yl]acrylic acid obtained in Reference Example 81 and N-butylsulfamide obtained in Reference Example 111.

¹H-NMR (300 MHz, CDCl₃) δ:0.87 (t, J=7.4 Hz, 3H), 1.27-1.38 (m, 2H), 1.42-1.51 (m, 2H), 2.46 (s, 3H), 2.93 (q, J=6.8 Hz, 2H), 3.53 (s, 3H), 5.06 (t, J=6.1 Hz, 1H), 5.33 (d, J=15.5 Hz, 1H), 6.93 (d, J=3.4 Hz, 1H), 7.07 (d, J=8.7 Hz, 1H), 7.23 (d, J=3.4 Hz, 1H), 7.38 (d, J=15.5 Hz, 1H), 7.51 (dd, J=8.3, 1.5 Hz, 1H), 8.10 (s, 1H).

### Example 151

### (2E)-N-[(butylamino)sulfonyl]-3-[5-(3-chloro-1H-pyrrolo[2,3-b]pyridin-1-yl)-1,3-dimethyl-1H-pyrazol-4-yl]acrylamide

By a method similar to that in Example 62, the title compound was obtained from (2E)-3-[5-(3-chloro-1H-pyrrolo[2,3-b]pyridin-1-yl)-1,3-dimethyl-1H-pyrazol-4-yl]acrylic acid obtained in Reference Example 133 and N-butylsulfamide obtained in Reference Example 111.

¹H-NMR (300 MHz, DMSO-d₆) δ:0.79 (t, J=7.2 Hz, 3H), 1.16-1.28 (m, 2H), 1.30-1.43 (m, 2H), 2.38 (s, 3H), 2.79 (q, J=6.4 Hz, 2H), 3.50 (s, 3H), 6.07 (d, J=15.9 Hz, 1H), 7.02 (d, J=15.9 Hz, 1H), 7.40 (dd, J=7.6, 4.5 Hz, 1H), 7.48-7.56 (m, 1H), 8.06 (s, 1H), 8.16 (d, J=8.0 Hz, 1H), 8.38 (d, J=4.2 Hz, 1H), 11.30 (br s, 1H).

### Example 152

### (2E)-N-[(butylamino)sulfonyl]-3-[5-(5-fluoro-1H-indol-1-yl)-1,3-dimethyl-1H-pyrazol-4-yl]acrylamide

By a method similar to that in Example 62, the title compound was obtained from (2E)-3-[5-(5-fluoro-1H-indol-1-yl)-1,3-dimethyl-1H-pyrazol-4-yl]acrylic acid obtained in Reference Example 21 and N-butylsulfamide obtained in Reference Example 111.

¹H-NMR (300 MHz, CDCl₃) δ:0.84-0.90 (m, 3H), 1.24-1.38 (m, 2H), 1.41-1.52 (m, 2H), 2.42 (s, 3H), 2.91 (q, J=6.7 Hz, 2H), 3.53 (s, 3H), 5.13 (t, J=6.1 Hz, 1H), 5.29 (d, J=15.5 Hz, 1H), 6.79 (d, J=3.4 Hz, 1H), 6.86-6.93 (m, 1H), 6.94-7.04 (m, 1H), 7.11 (d, J=3.4 Hz, 1H), 7.34-7.45 (m, 2H), 7.97 (br s, 1H).

### Example 153

### (2E)-N-{[(cyclopropylmethyl)amino]sulfonyl}-3-[5-(5-fluoro-1H-indol-1-yl)-1,3-dimethyl-1H-pyrazol-4-yl]acrylamide

By a method similar to that in Example 62, the title compound was obtained from (2E)-3-[5-(5-fluoro-1H-indol-1-yl)-1,3-dimethyl-1H-pyrazol-4-yl]acrylic acid obtained in Reference Example 21 and N-(cyclopropylmethyl)sulfamide obtained in Reference Example 115.

¹H-NMR (300 MHz, CDCl₃) δ:0.10-0.18 (m, 2H), 0.44-0.53 (m, 2H), 0.86-1.00 (m, 1H), 2.43 (s, 3H), 2.81 (t, J=6.1 Hz, 2H), 3.54 (s, 3H), 5.18-5.26 (m, 2H), 6.80 (d, J=3.4 Hz, 1H), 6.86-6.92 (m, 1H), 6.95-7.04 (m, 1H), 7.11 (d, J=3.0 Hz, 1H), 7.35-7.46 (m, 2H).

### Example 154

### (2E)-3-[5-(5-cyano-1H-indol-1-yl)-1,3-dimethyl-1H-pyrazol-4-yl]-N-{[(cyclopropylmethyl)amino]sulfonyl}acrylamide

By a method similar to that in Example 62, the title compound was obtained from (2E)-3-[5-(5-cyano-1H-indol-1-yl)-1,3-dimethyl-1H-pyrazol-4-yl]acrylic acid obtained in Reference Example 81 and N-(cyclopropylmethyl)sulfamide obtained in Reference Example 115.

¹H-NMR (300 MHz, CDCl₃) δ:0.11-0.17 (m, 2H), 0.50 (d, J=7.9 Hz, 2H), 0.86-0.99 (m, 1H), 2.45 (s, 3H), 2.81 (t, J=6.1 Hz, 2H), 3.53 (s, 3H), 5.23 (br s, 1H), 5.31 (d, J=15.8 Hz, 1H), 6.93 (d, J=3.4 Hz, 1H), 7.06 (d, J=8.7 Hz, 1H), 7.22 (d, J=3.4 Hz, 1H), 7.38 (d, J=15.8 Hz, 1H), 7.50 (dd, J=8.6, 1.4 Hz, 1H), 8.10 (s, 1H).

### Example 155

### (2E)-3-[5-(5-chloro-1H-indol-1-yl)-1,3-dimethyl-1H-pyrazol-4-yl]-N-[(4-hydroxypiperidin-1-yl)sulfonyl]acrylamide

To a solution of (2E)-3-[5-(5-chloro-1H-indol-1-yl)-1,3-dimethyl-1H-pyrazol-4-yl]-N-[(4-oxopiperidin-1-yl)sulfonyl]acrylamide obtained in Example 142 (301 mg) in a mixed solvent of tetrahydrofuran (5 mL) and methanol (1 mL) was added sodium borohydride (26.3 mg), and the mixture was stirred at room temperature for 1 hr. Water was added to this reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 20:80, v/v) to give the title compound (176 mg, yield 58%) as a colorless amorphous solid.

¹H-NMR (300 MHz, CDCl₃) δ:1.55-1.67 (m, 2H), 1.83-1.94 (m, 2H), 2.43 (s, 3H), 3.09-3.19 (m, 2H), 3.51 (s, 3H), 3.53-3.62 (m, 2H), 3.83 (br s, 1H), 5.36 (d, J=15.8 Hz, 1H), 6.77 (d, J=3.4 Hz, 1H), 6.90 (d, J=8.9 Hz, 1H), 7.10 (d, J=3.4 Hz, 1H), 7.21 (dd, J=8.8, 2.0 Hz, 1H), 7.41 (d, J=15.8 Hz, 1H), 7.70 (d, J=1.9 Hz, 1H), 7.76 (br s, 1H).

### Example 156

### (2E)-N-[(butylamino)sulfonyl]-3-[5-(3-chloro-1H-indazol-1-yl)-1,3-dimethyl-1H-pyrazol-4-yl]acrylamide

By a method similar to that in Example 62, the title compound was obtained from (2E)-3-[5-(3-chloro-1H-indazol-1-yl)-1,3-dimethyl-1H-pyrazol-4-yl]acrylic acid obtained in Reference Example 40 and N-butylsulfamide obtained in Reference Example 111.

¹H-NMR (300 MHz, CDCl₃) δ:0.88 (t, J=7.2 Hz, 3H), 1.25-1.39 (m, 2H), 1.42-1.54 (m, 2H), 2.45 (s, 3H), 2.93 (q, J=6.4 Hz, 2H), 3.60 (s, 3H), 5.11 (t, J=6.2 Hz, 1H), 5.53 (d, J=15.9 Hz, 1H), 7.12 (d, J=8.3 Hz, 1H), 7.36-7.45 (m, 2H), 7.54 (t, J=7.4 Hz, 1H), 7.83 (d, J=8.3 Hz, 1H).

### Example 157

### (2E)-3-[3-cyclopropyl-5-(5-fluoro-1H-indol-1-yl)-1-methyl-1H-pyrazol-4-yl]-N-{[(cyclopropylmethyl)amino]sulfonyl}acrylamide

By a method similar to that in Example 62, the title compound was obtained from (2E)-3-[3-cyclopropyl-5-(5-fluoro-1H-indol-1-yl)-1-methyl-1H-pyrazol-4-yl]acrylic acid obtained in Reference Example 155 and N-(cyclopropylmethyl)sulfamide obtained in Reference Example 115.

¹H-NMR (300 MHz, CDCl₃) δ:0.10-0.18 (m, 2H), 0.43-0.54 (m, 2H), 0.86-1.07 (m, 5H), 1.90-2.02 (m, 1H), 2.75-2.86 (m, 2H), 3.50 (s, 3H), 5.25 (br s, 1H), 5.40 (d, J=15.8 Hz, 1H), 6.79 (d, J=3.2 Hz, 1H), 6.86-6.93 (m, 1H), 6.94-7.03 (m, 1H), 7.11 (d, J=3.4 Hz, 1H), 7.38 (dd, J=9.0, 2.1 Hz, 1H), 7.54 (d, J=15.8 Hz, 1H).

### Example 158

### (2E)-3-[5-(5-fluoro-1H-indol-1-yl)-1,3-dimethyl-1H-pyrazol-4-yl]-N-[(propylamino)sulfonyl]acrylamide

By a method similar to that in Example 62, the title compound was obtained from (2E)-3-[5-(5-fluoro-1H-indol-1-yl)-1,3-dimethyl-1H-pyrazol-4-yl]acrylic acid obtained in Reference Example 21 and N-propylsulfamide obtained in Reference Example 127.

¹H-NMR (300 MHz, CDCl₃) δ:0.90 (t, J=7.3 Hz, 3H), 1.45-1.56 (m, 2H), 2.44 (s, 3H), 2.85-2.92 (m, 2H), 3.54 (s, 3H), 5.08 (br s, 1H), 5.24 (d, J=15.8 Hz, 1H), 6.80 (d, J=3.2 Hz, 1H), 6.87-6.94 (m, 1H), 6.96-7.04 (m, 1H), 7.12 (d, J=3.2 Hz, 1H), 7.34-7.48 (m, 2H).

### Example 159

### (2E)-N-[(butylamino)sulfonyl]-3-[5-(6-methoxy-1H-indol-1-yl)-1,3-dimethyl-1H-pyrazol-4-yl]acrylamide

By a method similar to that in Example 62, the title compound was obtained from (2E)-3-[5-(6-methoxy-1H-indol-1-yl)-1,3-dimethyl-1H-pyrazol-4-yl]acrylic acid obtained in Reference Example 60 and N-butylsulfamide obtained in Reference Example 111.

¹H-NMR (300 MHz, CDCl₃) δ:0.87 (t, J=7.2 Hz, 3H), 1.24-1.38 (m, 2H), 1.41-1.52 (m, 2H), 2.44 (s, 3H), 2.91 (q, J=6.8 Hz, 2H), 3.55 (s, 3H), 3.77 (s, 3H), 5.00 (br s, 1H), 5.22 (d, J=15.5 Hz, 1H), 6.40 (d, J=1.9 Hz, 1H), 6.75 (d, J=3.0 Hz, 1H), 6.90 (dd, J=8.5, 2.1 Hz, 1H), 6.95 (d, J=3.0 Hz, 1H), 7.47 (d, J=15.5 Hz, 1H), 7.60 (d, J=8.7 Hz, 1H).

### Example 160

### (2E)-3-[5-(5-chloro-1H-indol-1-yl)-1,3-dimethyl-1H-pyrazol-4-yl]-N-[(4-hydroxy-4-methylpiperidin-1-yl)sulfonyl]acrylamide

To a solution of (2E)-3-[5-(5-chloro-1H-indol-1-yl)-1,3-dimethyl-1H-pyrazol-4-yl]-N-[(4-oxopiperidin-1-yl)sulfonyl]acrylamide obtained in Example 142 (308 mg) in tetrahydrofuran (6 mL) was added methylmagnesium bromide (1M diethyl ether solution, 1.4 mL) with stirring, and the mixture was stirred at room temperature for 1 hr. A saturated aqueous ammonium chloride solution was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 25:75, v/v) to give the title compound (155 mg, yield 49%) as a colorless oil.

¹H-NMR (300 MHz, CDCl₃) δ:1.25 (s, 3H), 1.58-1.73 (m, 4H), 2.44 (s, 3H), 3.15-3.32 (m, 2H), 3.43-3.58 (m, 5H), 5.33 (d, J=15.8 Hz, 1H), 6.78 (d, J=3.4 Hz, 1H), 6.91 (d, J=8.5 Hz, 1H), 7.10 (d, J=3.4 Hz, 1H), 7.22 (dd, J=8.8, 1.8 Hz, 1H), 7.38-7.47 (m, 2H), 7.71 (d, J=1.9 Hz, 1H).

### Example 161

### (2E)-N-[(butylamino)sulfonyl]-3-[5-(3-chloro-6-methoxy-1H-indol-1-yl)-1,3-dimethyl-1H-pyrazol-4-yl]acrylamide

To a solution of (2E)-N-[(butylamino)sulfonyl]-3-[5-(6-methoxy-1H-indol-1-yl)-1,3-dimethyl-1H-pyrazol-4-yl]acrylamide obtained in Example 159 (248 mg) in acetonitrile (2.5 mL) was added N-chlorosuccinimide (76 mg), and the mixture was stirred at room temperature for 24 hr. Ethyl acetate was added to the reaction mixture, and the organic layer was washed with water and saturated brine, dried over anhydrous magnesium sulfate, and filtrated. The filtrate was concentrated, and the residue was subjected to silica gel column chromatography (hexane-ethyl acetate 95:5-60:40, v/v). The obtained solid was crystallized from hexane-ethyl acetate, and then water-ethanol. The obtained crystals were purified by preparative HPLC (tool and preparative conditions were the same as those in Reference Example 97), and the eluate was concentrated. The obtained oil was dissolved in ethyl acetate, and the organic layer was washed with saturated brine, dried over anhydrous magnesium sulfate, and filtrated. The filtrate was concentrated, and the residue was crystallized from hexane-ethyl acetate to give the title compound (43 mg, yield 16%) as colorless crystals.

¹H-NMR (300 MHz, DMSO-d₆) δ:0.79 (t, J=7.2 Hz, 3H), 1.16-1.28 (m, 2H), 1.29-1.44 (m, 2H), 2.39 (s, 3H), 2.78 (q, J=6.8 Hz, 2H), 3.50 (s, 3H), 3.70 (s, 3H), 6.10 (d, J=16.3 Hz, 1H), 6.52 (d, J=1.9 Hz, 1H), 6.95 (dd, J=8.7, 1.9 Hz, 1H), 7.03 (d, J=15.9 Hz, 1H), 7.44-7.59 (m, 1H), 7.54 (d, J=8.7 Hz, 1H), 7.72 (s, 1H), 11.33 (s, 1H)

### Example 162

### (2E)-N-[(butylamino)sulfonyl]-3-{1,3-dimethyl-5-[6-(2-oxopropoxy)-1H-indol-1-yl]-1H-pyrazol-4-yl}acrylamide

To a solution of ethyl (2E)-3-{1,3-dimethyl-5-[6-(2-oxopropoxy)-1H-indol-1-yl]-1H-pyrazol-4-yl}acrylate obtained in Reference Example 157 (2.25 g) in a mixed solvent of tetrahydrofuran (10 mL) and ethanol (10 mL) was added a 1N aqueous sodium hydroxide solution (12 mL), and the mixture was stirred with heating at 50° C. for 5 hr. The reaction mixture was allowed to cool to room temperature, and concentrated under reduced pressure. The residue was neutralized with an aqueous solution (10 mL) of potassium hydrogensulfate (1.6 g), and the precipitated crystals were collected by filtration. The obtained crystals were dissolved in ethyl acetate and tetrahydrofuran, and the solution was dried over anhydrous magnesium sulfate, and filtrated. The filtrate was concentrated, and the residue was subjected to silica gel column chromatography (methanol-ethyl acetate 5:95, v/v) to give a pale-yellow amorphous solid.

The obtained amorphous solid was dissolved in acetonitrile (40 mL), 2-methyl-6-nitrobenzoic anhydride (1.68 g), N-butylsulfamide obtained in Reference Example 111 (651 mg), triethylamine (1.23 g) and 4-dimethylaminopyridine (497 mg) were added, and the mixture was stirred at room temperature for 48 hr. A saturated aqueous ammonium chloride solution (20 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 35:65, v/v), and crystallized from hexane-ethyl acetate to give the title compound (245 mg, yield 85%) as colorless crystals.

¹H-NMR (300 MHz, CDCl₃) δ:0.87 (t, J=7.2 Hz, 3H), 1.23-1.37 (m, 2H), 1.40-1.51 (m, 2H), 2.26 (s, 3H), 2.45 (s, 3H), 2.93 (d, J=6.6 Hz, 2H), 3.54 (s, 3H), 4.53 (s, 2H), 5.02 (br s, 1H), 5.27 (d, J=15.8 Hz, 1H), 6.39-6.43 (m, 1H), 6.76 (d, J=3.0 Hz, 1H), 6.91 (dd, J=8.6, 2.2 Hz, 1H), 6.98 (d, J=3.2 Hz, 1H), 7.44 (d, J=15.8 Hz, 1H), 7.62 (d, J=8.7 Hz, 1H).

### Example 163

### (2E)-3-[5-(5-chloro-1H-indol-1-yl)-1,3-dimethyl-1H-pyrazol-4-yl]-N-{[(3-hydroxy-3-methylbutyl)amino]sulfonyl}acrylamide

To a solution of ethyl N-[({(2E)-3-[5-(5-chloro-1H-indol-1-yl)-1,3-dimethyl-1H-pyrazol-4-yl]prop-2-enoyl}amino)sulfonyl]-O-alaninate obtained in Example 143 (380 mg) in tetrahydrofuran (8 mL) was added methylmagnesium bromide (1M diethyl ether solution, 5 mL) with stirring, and the mixture was stirred at room temperature for 16 hr. A saturated aqueous ammonium chloride solution was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 35:65, v/v) to give the title compound (130 mg, yield 35%) as a colorless amorphous solid.

¹H-NMR (300 MHz, CDCl₃) δ:1.19 (d, J=2.7 Hz, 6H), 1.65 (t, J=6.6 Hz, 2H), 2.43 (s, 3H), 3.09-3.18 (m, 2H), 3.52 (s, 3H), 5.28-5.35 (m, 1H), 6.03 (br s, 1H), 6.78 (d, J=2.7 Hz, 1H), 6.90 (d, J=8.7 Hz, 1H), 7.11 (d, J=3.0 Hz, 1H), 7.20 (dd, J=8.7, 1.9 Hz, 1H), 7.38-7.44 (m, 1H), 7.70 (d, J=1.9 Hz, 1H).

### Example 164

### (2E)-N-[(butylamino)sulfonyl]-3-{5-[6-(2-methoxyethoxy)-1H-indol-1-yl]-1,3-dimethyl-1H-pyrazol-4-yl}acrylamide

By a method similar to that in Example 62, the title compound was obtained from (2E)-3-{5-[6-(2-methoxyethoxy)-1H-indol-1-yl]-1,3-dimethyl-1H-pyrazol-4-yl}acrylic acid obtained in Reference Example 159 and N-butylsulfamide obtained in Reference Example 111.

¹H-NMR (300 MHz, CDCl₃) δ:0.87 (t, J=7.3 Hz, 3H), 1.23-1.38 (m, 2H), 1.41-1.52 (m, 2H), 2.42 (s, 3H), 2.91 (q, J=6.7 Hz, 2H), 3.42 (s, 3H), 3.53 (s, 3H), 3.68-3.78 (m, 2H), 4.02-4.10 (m, 2H), 5.05 (br s, 1H), 5.26 (d, J=15.8 Hz, 1H), 6.46 (d, J=1.3 Hz, 1H), 6.74 (d, J=2.8 Hz, 1H), 6.88-6.98 (m, 2H), 7.45 (d, J=15.8 Hz, 1H), 7.58 (d, J=8.7 Hz, 1H), 7.91 (br s, 1H).

### Example 165

### (2E)-3-{5-[6-(2-methoxyethoxy)-1H-indol-1-yl]-1,3-dimethyl-1H-pyrazol-4-yl}-N-[(propylamino)sulfonyl]acrylamide

By a method similar to that in Example 62, the title compound was obtained from (2E)-3-{5-[6-(2-methoxyethoxy)-1H-indol-1-yl]-1,3-dimethyl-1H-pyrazol-4-yl}acrylic acid obtained in Reference Example 159 and N-propylsulfamide obtained in Reference Example 127.

¹H-NMR (300 MHz, CDCl₃) δ:0.90 (t, J=7.3 Hz, 3H), 1.46-1.54 (m, 2H), 2.43 (s, 3H), 2.89 (q, J=6.8 Hz, 2H), 3.43 (s, 3H), 3.54 (s, 3H), 3.70-3.75 (m, 2H), 4.03-4.11 (m, 2H), 5.03 (br s, 1H), 5.20 (d, J=15.8 Hz, 1H), 6.46 (d, J=1.7 Hz, 1H), 6.75 (d, J=3.0 Hz, 1H), 6.91-6.97 (m, 2H), 7.46 (d, J=15.6 Hz, 1H), 7.59 (d, J=8.7 Hz, 1H), 7.70 (br s, 1H).

### Example 166

### (2E)-N-[(butylamino)sulfonyl]-3-[5-(6-hydroxy-1H-indol-1-yl)-1,3-dimethyl-1H-pyrazol-4-yl]acrylamide

To a solution of (2E)-N-[(butylamino)sulfonyl]-3-[5-(6-methoxy-1H-indol-1-yl)-1,3-dimethyl-1H-pyrazol-4-yl]acrylamide obtained in Example 159 (1.65 g) in dichloromethane (30 mL) was added dropwise boron tribromide (1M dichloromethane solution, 7.4 mL) with stirring at −78° C., and the mixture was stirred at −78° C. for 1 hr, and then at room temperature for 17 hr. The reaction mixture was quenched with methanol (10 mL), and concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v), and crystallized from hexane-ethanol to give the title compound (1.44 g, yield 91%) as colorless crystals.

¹H-NMR (300 MHz, DMSO-d₆) δ:0.80 (t, J=7.3 Hz, 3H), 1.16-1.29 (m, 2H), 1.31-1.42 (m, 2H), 2.39 (s, 3H), 2.80 (q, J=6.8 Hz, 2H), 3.46 (s, 3H), 6.15 (d, J=16.0 Hz, 1H), 6.30 (d, J=1.9 Hz, 1H), 6.67-6.72 (m, 1H), 7.02 (d, J=16.0 Hz, 1H), 7.33 (d, J=3.4 Hz, 1H), 7.45-7.55 (m, 2H), 9.22 (s, 1H), 11.32 (s, 1H).

### Example 167

### (2E)-N-[(butylamino)sulfonyl]-3-{5-[6-(cyclopropylmethoxy)-1H-indol-1-yl]-1,3-dimethyl-1H-pyrazol-4-yl}acrylamide

By a method similar to that in Example 62, the title compound was obtained from (2E)-3-{5-[6-(cyclopropylmethoxy)-1H-indol-1-yl]-1,3-dimethyl-1H-pyrazol-4-yl}acrylic acid obtained in Reference Example 161 and N-butylsulfamide obtained in Reference Example 111.

¹H-NMR (300 MHz, CDCl₃) δ:0.33 (d, J=4.7 Hz, 2H), 0.63 (d, J=7.3 Hz, 2H), 0.87 (t, J=7.2 Hz, 3H), 1.20-1.37 (m, 3H), 1.45 (d, J=7.3 Hz, 2H), 2.43 (s, 3H), 2.85-2.97 (m, 2H), 3.54 (s, 3H), 3.74 (d, J=6.8 Hz, 2H), 5.02 (br s, 1H), 5.23 (d, J=15.8 Hz, 1H), 6.40 (s, 1H), 6.74 (d, J=2.8 Hz, 1H), 6.87-6.97 (m, 2H), 7.46 (d, J=15.6 Hz, 1H), 7.58 (d, J=8.5 Hz, 1H), 7.72 (br s, 1H).

### Example 168

### (2E)-N-{[(cyclopropylmethyl)amino]sulfonyl}-3-[5-(6-isopropoxy-1H-indol-1-yl)-1,3-dimethyl-1H-pyrazol-4-yl]acrylamide

By a method similar to that in Example 62, the title compound was obtained from (2E)-3-[5-(6-isopropoxy-1H-indol-1-yl)-1,3-dimethyl-1H-pyrazol-4-yl]acrylic acid obtained in Reference Example 163 and N-(cyclopropylmethyl)sulfamide obtained in Reference Example 115.

¹H-NMR (300 MHz, CDCl₃) δ:0.14 (d, J=4.7 Hz, 2H), 0.45-0.53 (m, 2H), 0.86-0.98 (m, 1H), 1.27-1.32 (m, 6H), 2.43 (s, 3H), 2.80 (t, J=6.2 Hz, 2H), 3.55 (s, 3H), 4.43-4.54 (m, 1H), 5.18-5.25 (m, 2H), 6.42 (d, J=1.9 Hz, 1H), 6.72-6.75 (m, 1H), 6.88 (dd, J=8.7, 2.1 Hz, 1H), 6.94 (d, J=3.4 Hz, 1H), 7.46 (d, J=15.8 Hz, 1H), 7.58 (d, J=8.7 Hz, 1H).

### Example 169

### 1-[4-((1E)-3-{[(butylamino)sulfonyl]amino}-3-oxoprop-1-en-1-yl)-1,3-dimethyl-1H-pyrazol-5-yl]-1H-indol-6-yl methanesulfonate

To a solution of (2E)-N-[(butylamino)sulfonyl]-3-[5-(6-hydroxy-1H-indol-1-yl)-1,3-dimethyl-1H-pyrazol-4-yl]acrylamide obtained in Example 166 (335 mg) in tetrahydrofuran (2 ml) were added triethylamine (118 mg) and methanesulfonyl chloride (124 mg), and the mixture was stirred at room temperature for 4 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v), and crystallized from diethyl ether-ethanol to give the title compound (284 mg, yield 72%) as colorless crystals.

¹H-NMR (300 MHz, CDCl₃) δ:0.88 (t, J=7.2 Hz, 3H), 1.24-1.38 (m, 2H), 1.42-1.54 (m, 2H), 2.42 (s, 3H), 2.94 (q, J=6.4 Hz, 2H), 3.15 (s, 3H), 3.57 (s, 3H), 5.15 (t, J=6.1 Hz, 1H), 5.48 (d, J=15.9 Hz, 1H), 6.85 (d, J=2.7 Hz, 1H), 7.01 (d, J=1.5 Hz, 1H), 7.12-7.18 (m, 2H), 7.34 (d, J=15.9 Hz, 1H), 7.74 (d, J=8.7 Hz, 1H), 8.24 (br s, 1H).

### Example 170

### (2E)-N-[(butylamino)sulfonyl]-3-[5-(6-isopropoxy-1H-indol-1-yl)-1,3-dimethyl-1H-pyrazol-4-yl]acrylamide

By a method similar to that in Example 62, the title compound was obtained from (2E)-3-[5-(6-isopropoxy-1H-indol-1-yl)-1,3-dimethyl-1H-pyrazol-4-yl]acrylic acid obtained in Reference Example 163 and N-butylsulfamide obtained in Reference Example 111.

¹H-NMR (300 MHz, CDCl₃) δ:0.87 (t, J=7.3 Hz, 3H), 1.25-1.38 (m, 8H), 1.42-1.53 (m, 2H), 2.43 (s, 3H), 2.91 (q, J=6.8 Hz, 2H), 3.55 (s, 3H), 4.43-4.54 (m, 1H), 5.00-5.08 (m, 1H), 5.25 (d, J=15.8 Hz, 1H), 6.43 (d, J=1.9 Hz, 1H), 6.74 (d, J=3.2 Hz, 1H), 6.88 (dd, J=8.6, 2.2 Hz, 1H), 6.94 (d, J=3.4 Hz, 1H), 7.46 (d, J=15.8 Hz, 1H).

### Example 171

### (2E)-3-[5-(5-chloro-1H-indol-1-yl)-1,3-dimethyl-1H-pyrazol-4-yl]-N-[(propoxyamino)sulfonyl]acrylamide

By a method similar to that in Example 62, the title compound was obtained from (2E)-3-[5-(5-chloro-1H-indol-1-yl)-1,3-dimethyl-1H-pyrazol-4-yl]acrylic acid obtained in Reference Example 38 and N-propoxysulfamide obtained in Reference Example 165.

¹H-NMR (300 MHz, CDCl₃) δ:0.87 (t, J=7.3 Hz, 3H), 1.58-1.64 (m, 2H), 2.44 (s, 3H), 3.53 (s, 3H), 3.88 (t, J=6.8 Hz, 2H), 5.28 (d, J=15.8 Hz, 1H), 6.79 (d, J=3.0 Hz, 1H), 6.91 (d, J=8.7 Hz, 1H), 7.10 (d, J=3.4 Hz, 1H), 7.20-7.25 (m, 1H), 7.46 (d, J=15.8 Hz, 1H), 7.72 (d, J=1.5 Hz, 1H), 7.81 (br s, 1H).

### Example 172

### (2E)-N-{[(cyclopropylmethyl)amino]sulfonyl}-3-[5-(6-ethoxy-1H-indol-1-yl)-1,3-dimethyl-1H-pyrazol-4-yl]acrylamide

By a method similar to that in Example 62, the title compound was obtained from (2E)-3-[5-(6-ethoxy-1H-indol-1-yl)-1,3-dimethyl-1H-pyrazol-4-yl]acrylic acid obtained in Reference Example 167 and N-(cyclopropylmethyl)sulfamide obtained in Reference Example 115.

¹H-NMR (300 MHz, CDCl₃) δ:0.11-0.17 (m, 2H), 0.45-0.52 (m, 2H), 0.86-0.98 (m, 1H), 1.39 (t, J=7.0 Hz, 3H), 2.44 (s, 3H), 2.80 (t, J=6.4 Hz, 2H), 3.55 (s, 3H), 3.96 (q, J=7.0 Hz, 2H), 5.13-5.24 (m, 2H), 6.40 (d, J=1.7 Hz, 1H), 6.74 (d, J=3.4 Hz, 1H), 6.86-6.96 (m, 2H), 7.46 (d, J=15.6 Hz, 1H), 7.59 (d, J=8.7 Hz, 1H), 7.68 (br s, 1H).

### Example 173

### (2E)-N-[(butylamino)sulfonyl]-3-{5-[6-(2-methoxy-1-methylethoxy)-1H-indol-1-yl]-1,3-dimethyl-1H-pyrazol-4-yl}acrylamide

By a method similar to that in Example 62, the title compound was obtained from (2E)-3-{5-[6-(2-methoxy-1-methylethoxy)-1H-indol-1-yl]-1,3-dimethyl-1H-pyrazol-4-yl}acrylic acid obtained in Reference Example 172 and N-butylsulfamide obtained in Reference Example 111.

¹H-NMR (300 MHz, CDCl₃) δ:0.87 (t, J=7.3 Hz, 3H), 1.23-1.38 (m, 5H), 1.41-1.53 (m, 2H), 2.42 (s, 3H), 2.92 (q, J=6.8 Hz, 2H), 3.37 (d, J=0.9 Hz, 3H), 3.42-3.60 (m, 5H), 4.43-4.54 (m, 1H), 5.04 (br s, 1H), 5.25 (dd, J=15.8, 2.8 Hz, 1H), 6.50 (d, J=1.7 Hz, 1H), 6.73 (d, J=3.0 Hz, 1H), 6.89-6.96 (m, 2H), 7.46 (d, J=15.6 Hz, 1H), 7.58 (d, J=8.7 Hz, 1H), 7.88 (br s, 1H).

### Example 174

### (2E)-N-{[(cyclopropylmethyl)amino]sulfonyl}-3-[1,3-dimethyl-5-(5-methyl-1H-indol-1-yl)-1H-pyrazol-4-yl]acrylamide

By a method similar to that in Example 62, the title compound was obtained from (2E)-3-[1,3-dimethyl-5-(5-methyl-1H-indol-1-yl)-1H-pyrazol-4-yl]acrylic acid obtained in Reference Example 175 and N-(cyclopropylmethyl)sulfamide obtained in Reference Example 115.

¹H-NMR (300 MHz, CDCl₃) δ:0.11-0.17 (m, 2H), 0.45-0.52 (m, 2H), 0.86-0.96 (m, 1H), 2.43 (s, 3H), 2.48 (s, 3H), 2.80 (t, J=6.5 Hz, 2H), 3.54 (s, 3H), 5.10-5.21 (m, 2H), 6.73-6.76 (m, 1H), 6.87 (d, J=8.5 Hz, 1H), 7.02 (d, J=3.4 Hz, 1H), 7.08 (d, J=8.7 Hz, 1H), 7.45 (d, J=15.8 Hz, 1H), 7.52 (s, 1H).

### Example 175

### (2E)-N-[(butylamino)sulfonyl]-3-[1,3-dimethyl-5-(5-methyl-1H-indol-1-yl)-1H-pyrazol-4-yl]acrylamide

By a method similar to that in Example 62, the title compound was obtained from (2E)-3-[1,3-dimethyl-5-(5-methyl-1H-indol-1-yl)-1H-pyrazol-4-yl]acrylic acid obtained in Reference Example 175 and N-butylsulfamide obtained in Reference Example 111.

¹H-NMR (300 MHz, CDCl₃) δ:0.87 (t, J=7.2 Hz, 3H), 1.25-1.37 (m, 2H), 1.45 (d, J=7.2 Hz, 2H), 2.42 (s, 3H), 2.48 (s, 3H), 2.90 (q, J=6.3 Hz, 2H), 3.53 (s, 3H), 5.04 (br s, 1H), 5.22 (d, J=15.8 Hz, 1H), 6.74 (d, J=2.8 Hz, 1H), 6.87 (d, J=8.1 Hz, 1H), 7.02 (d, J=3.0 Hz, 1H), 7.08 (d, J=8.3 Hz, 1H), 7.45 (d, J=15.8 Hz, 1H), 7.51 (s, 1H), 7.75 (br s, 1H).

### Example 176

### (2E)-N-[(butylamino)sulfonyl]-3-[5-(6-ethoxy-1H-indol-1-yl)-1,3-dimethyl-1H-pyrazol-4-yl]acrylamide

By a method similar to that in Example 62, the title compound was obtained from (2E)-3-[5-(6-ethoxy-1H-indol-1-yl)-1,3-dimethyl-1H-pyrazol-4-yl]acrylic acid obtained in Reference Example 167 and N-butylsulfamide obtained in Reference Example 111.

¹H-NMR (300 MHz, CDCl₃) δ:0.87 (t, J=7.3 Hz, 3H), 1.24-1.52 (m, 7H), 2.43 (s, 3H), 2.91 (q, J=6.6 Hz, 2H), 3.54 (s, 3H), 3.96 (q, J=7.0 Hz, 2H), 5.00-5.10 (m, 1H), 5.25 (d, J=15.6 Hz, 1H), 6.40 (s, 1H), 6.74 (d, J=3.0 Hz, 1H), 6.85-6.96 (m, 2H), 7.46 (d, J=15.6 Hz, 1H), 7.58 (d, J=8.7 Hz, 1H), 7.84 (br s, 1H).

### Example 177

### (2E)-3-{1,3-dimethyl-5-[6-(trifluoromethyl)-1H-indol-1-yl]-1H-pyrazol-4-yl}-N-[(propylamino)sulfonyl]acrylamide

By a method similar to that in Example 62, the title compound was obtained from (2E)-3-{1,3-dimethyl-5-[6-(trifluoromethyl)-1H-indol-1-yl]-1H-pyrazol-4-yl}acrylic acid obtained in Reference Example 42 and N-propylsulfamide obtained in Reference Example 127.

¹H-NMR (300 MHz, CDCl₃) δ:0.88 (t, J=7.3 Hz, 3H), 1.44-1.55 (m, 2H), 2.47 (s, 3H), 2.87 (q, J=6.8 Hz, 2H), 3.54 (s, 3H), 5.09-5.17 (m, 1H), 5.30 (d, J=15.8 Hz, 1H), 6.89-6.94 (m, 1H), 7.22-7.27 (m, 2H), 7.40 (d, J=15.8 Hz, 1H), 7.48-7.53 (m, 1H), 7.85 (d, J=8.3 Hz, 1H).

### Example 178

### (2E)-3-[5-(5-chloro-1H-pyrrolo[2,3-b]pyridin-1-yl)-1,3-dimethyl-1H-pyrazol-4-yl]-N-[(cyclohexylamino)sulfonyl]acrylamide

By a method similar to that in Example 62, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and N-cyclohexylsulfamide obtained in Reference Example 114.

¹H-NMR (300 MHz, DMSO-d₆) δ:1.01-1.20 (m, 5H), 1.42-1.52 (m, 1H), 1.57-1.74 (m, 4H), 2.38 (s, 3H), 3.01 (br s, 1H), 3.48 (s, 3H), 6.01 (d, J=16.0 Hz, 1H), 6.88 (d, J=3.6 Hz, 1H), 7.00 (d, J=16.0 Hz, 1H), 7.56 (br s, 1H), 7.83 (d, J=3.6 Hz, 1H), 8.28 (d, J=2.3 Hz, 1H), 8.32 (d, J=2.1 Hz, 1H), 11.27 (s, 1H).

### Example 179

### (2E)-N-[(butylamino)sulfonyl]-3-{1,3-dimethyl-5-[6-(trifluoromethyl)-1H-indol-1-yl]-1H-pyrazol-4-yl}acrylamide

By a method similar to that in Example 62, the title compound was obtained from (2E)-3-{1,3-dimethyl-5-[6-(trifluoromethyl)-1H-indol-1-yl]-1H-pyrazol-4-yl}acrylic acid obtained in Reference Example 42 and N-butylsulfamide obtained in Reference Example 111.

¹H-NMR (300 MHz, CDCl₃) δ:0.86 (t, J=7.3 Hz, 3H), 1.23-1.37 (m, 2H), 1.40-1.52 (m, 2H), 2.46 (s, 3H), 2.90 (q, J=6.7 Hz, 2H), 3.54 (s, 3H), 5.04-5.13 (m, 1H), 5.30 (d, J=15.8 Hz, 1H), 6.91 (d, J=3.2 Hz, 1H), 7.22-7.28 (m, 2H), 7.40 (d, J=15.8 Hz, 1H), 7.50 (d, J=8.3 Hz, 1H), 7.85 (d, J=8.3 Hz, 1H).

### Example 180

### (2E)-N-{[(cyclopropylmethyl)amino]sulfonyl}-3-{1,3-dimethyl-5-[6-(trifluoromethyl)-1H-indol-1-yl]-1H-pyrazol-4-yl}acrylamide

By a method similar to that in Example 62, the title compound was obtained from (2E)-3-{1,3-dimethyl-5-[6-(trifluoromethyl)-1H-indol-1-yl]-1H-pyrazol-4-yl}acrylic acid obtained in Reference Example 42 and N-(cyclopropylmethyl)sulfamide obtained in Reference Example 115.

¹H-NMR (300 MHz, CDCl₃) δ:0.10-0.16 (m, 2H), 0.42-0.52 (m, 2H), 0.83-0.98 (m, 1H), 2.47 (s, 3H), 2.75-2.86 (m, 2H), 3.54 (s, 3H), 5.18-5.29 (m, 2H), 6.91 (d, J=3.4 Hz, 1H), 7.22-7.27 (m, 2H), 7.41 (d, J=15.8 Hz, 1H), 7.51 (d, J=7.9 Hz, 1H), 7.85 (d, J=8.3 Hz, 1H).

### Example 181

### (2E)-3-[5-(6-chloro-1H-indol-1-yl)-1,3-dimethyl-1H-pyrazol-4-yl]-N-[(propylamino)sulfonyl]acrylamide

By a method similar to that in Example 62, the title compound was obtained from (2E)-3-[5-(6-chloro-1H-indol-1-yl)-1,3-dimethyl-1H-pyrazol-4-yl]acrylic acid obtained in Reference Example 25 and N-propylsulfamide obtained in Reference Example 127.

¹H-NMR (300 MHz, CDCl₃) δ:0.87-0.93 (m, 3H), 1.45-1.56 (m, 2H), 2.45 (s, 3H), 2.85-2.94 (m, 2H), 3.54 (s, 3H), 5.09 (br s, 1H), 5.24 (d, J=15.8 Hz, 1H), 6.82 (dd, J=3.3, 0.8 Hz, 1H), 6.97-6.99 (m, 1H), 7.07 (d, J=3.4 Hz, 1H), 7.23 (dd, J=8.5, 1.9 Hz, 1H), 7.42 (d, J=15.6 Hz, 1H), 7.65 (d, J=8.3 Hz, 1H).

### Example 182

### (2E)-3-[5-(5-fluoro-1H-indol-1-yl)-1,3-dimethyl-1H-pyrazol-4-yl]-N-(piperidin-1-ylsulfonyl)acrylamide

By a method similar to that in Example 1, the title compound was obtained from (2E)-3-[5-(5-fluoro-1H-indol-1-yl)-1,3-dimethyl-1H-pyrazol-4-yl]acrylic acid obtained in Reference Example 21 and piperidine-1-sulfonamide obtained in Reference Example 177.

¹H-NMR (300 MHz, CDCl₃) δ:1.50 (br s, 2H), 1.56-1.63 (m, 4H), 2.43 (s, 3H), 3.22-3.30 (m, 4H), 3.52 (s, 3H), 5.37 (d, J=15.6 Hz, 1H), 6.79 (d, J=2.6 Hz, 1H), 6.86-6.93 (m, 1H), 6.95-7.04 (m, 1H), 7.11 (d, J=3.2 Hz, 1H), 7.35-7.46 (m, 2H), 7.68 (br s, 1H).

### Example 183

### (2E)-3-[5-(6-chloro-1H-indol-1-yl)-1,3-dimethyl-1H-pyrazol-4-yl]-N-{[(cyclopropylmethyl)amino]sulfonyl}acrylamide

By a method similar to that in Example 62, the title compound was obtained from (2E)-3-[5-(6-chloro-1H-indol-1-yl)-1,3-dimethyl-1H-pyrazol-4-yl]acrylic acid obtained in Reference Example 25 and N-(cyclopropylmethyl)sulfamide obtained in Reference Example 115.

¹H-NMR (300 MHz, CDCl₃) δ:0.12-0.18 (m, 2H), 0.45-0.52 (m, 2H), 0.84-0.98 (m, 1H), 2.44 (s, 3H), 2.78-2.86 (m, 2H), 3.54 (s, 3H), 5.18-5.27 (m, 2H), 6.81 (d, J=3.4 Hz, 1H), 6.97 (s, 1H), 7.07 (d, J=3.4 Hz, 1H), 7.23 (dd, J=8.5, 1.7 Hz, 1H), 7.42 (d, J=15.8 Hz, 1H), 7.65 (d, J=8.3 Hz, 1H).

### Example 184

### (2E)-N-[(butylamino)sulfonyl]-3-[5-(6-chloro-1H-indol-1-yl)-1,3-dimethyl-1H-pyrazol-4-yl]acrylamide

By a method similar to that in Example 62, the title compound was obtained from (2E)-3-[5-(6-chloro-1H-indol-1-yl)-1,3-dimethyl-1H-pyrazol-4-yl]acrylic acid obtained in Reference Example 25 and N-butylsulfamide obtained in Reference Example 111.

¹H-NMR (300 MHz, CDCl₃) δ:0.84-0.91 (m, 3H), 1.24-1.38 (m, 2H), 1.41-1.53 (m, 2H), 2.44 (s, 3H), 2.92 (q, J=6.8 Hz, 2H), 3.54 (s, 3H), 5.10 (br s, 1H), 5.27 (d, J=15.8 Hz, 1H), 6.81 (dd, J=3.3, 0.8 Hz, 1H), 6.96-6.99 (m, 1H), 7.07 (d, J=3.2 Hz, 1H), 7.23 (dd, J=8.4, 1.8 Hz, 1H), 7.42 (d, J=15.8 Hz, 1H), 7.65 (d, J=8.5 Hz, 1H).

### Example 185

### (2E)-3-[5-(5-chloro-1H-indol-1-yl)-1,3-dimethyl-1H-pyrazol-4-yl]-N-(piperidin-1-ylsulfonyl)acrylamide

By a method similar to that in Example 1, the title compound was obtained from (2E)-3-[5-(5-chloro-1H-indol-1-yl)-1,3-dimethyl-1H-pyrazol-4-yl]acrylic acid obtained in Reference Example 38 and piperidine-1-sulfonamide obtained in Reference Example 177.

¹H-NMR (300 MHz, CDCl₃) δ:1.48-1.55 (m, 2H), 1.56-1.65 (m, 4 H), 2.43 (s, 3H), 3.25 (t, J=5.2 Hz, 4H), 3.51 (s, 3H), 5.38 (d, J=15.8 Hz, 1H), 6.77 (d, J=2.4 Hz, 1H), 6.90 (d, J=8.7 Hz, 1H), 7.10 (d, J=3.2 Hz, 1H), 7.21 (dd, J=8.7, 2.1 Hz, 1H), 7.41 (d, J=15.8 Hz, 1H), 7.66-7.73 (m, 2H).

### Example 186

### butyl ({2-[5-(5-chloro-1H-indol-1-yl)-1,3-dimethyl-1H-pyrazol-4-yl]ethyl}sulfonyl)carbamate

To a solution of butanol (88.2 mg) in N,N-dimethylformamide (10 mL) was added N,N′-carbonyldiimidazole (209 mg), and the mixture was stirred at 60° C. for 1 hr. 2-[5-(5-Chloro-1H-indol-1-yl)-1,3-dimethyl-1H-pyrazol-4-yl]ethanesulfonamide obtained in Reference Example 179 (350 mg), 1,8-diazabicyclo[5.4.0]undec-7-ene (226 mg) and 4-dimethylaminopyridine (181 mg) were added to the reaction mixture, and the mixture was stirred at 60° C. for 20 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 50:50, v/v), and crystallized from hexane-ethyl acetate to give the title compound (299 mg, yield 67%) as colorless crystals.

¹H-NMR (300 MHz, CDCl₃) δ:0.93 (t, J=7.4 Hz, 3H), 1.22-1.41 (m, 2H), 1.51-1.62 (m, 2H), 2.31 (s, 3H), 2.65-2.87 (m, 2H), 3.30 (t, J=8.0 Hz, 2H), 3.46 (s, 3H), 4.05 (t, J=6.8 Hz, 2H), 6.70 (d, J=3.4 Hz, 1H), 6.89-6.94 (m, 2H), 7.12 (d, J=3.4 Hz, 1H), 7.20 (dd, J=8.7, 1.9 Hz, 1H), 7.67 (d, J=1.9 Hz, 1H).

### Example 187

### 3,3,3-trifluoropropyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, 3,3,3-trifluoropropan-1-ol and N,N′-carbonyldiimidazole.

¹H-NMR (300 MHz, DMSO-d₆) δ:2.20 (s, 3H), 2.38-2.47 (m, 1H), 2.54-2.72 (m, 3H), 3.21-3.29 (m, 2H), 3.38 (s, 3H), 4.19 (t, J=5.9 Hz, 2H), 6.77 (d, J=3.2 Hz, 1H), 7.04 (d, J=8.9 Hz, 1H), 7.20 (dd, J=8.8, 2.0 Hz, 1H), 7.59 (d, J=3.4 Hz, 1H), 7.75 (d, J=1.9 Hz, 1H), 11.69 (br s, 1H).

### Example 188

### butyl ({2-[5-(6-chloro-1H-indol-1-yl)-1,3-dimethyl-1H-pyrazol-4-yl]ethyl}sulfonyl)carbamate

By a method similar to that in Example 186, the title compound was obtained from 2-[5-(6-chloro-1H-indol-1-yl)-1,3-dimethyl-1H-pyrazol-4-yl]ethanesulfonamide obtained in Reference Example 181, butanol and N,N′-carbonyldiimidazole.

¹H-NMR (300 MHz, DMSO-d₆) δ:0.93 (t, J=7.3 Hz, 3H), 1.25-1.41 (m, 2H), 1.51-1.62 (m, 2H), 2.31 (s, 3H), 2.65-2.90 (m, 2H), 3.32 (t, J=7.9 Hz, 2H), 3.47 (s, 3H), 4.07 (t, J=6.7 Hz, 2H), 6.73 (dd, J=3.3, 0.8 Hz, 1H), 6.97-6.99 (m, 1H), 7.09 (d, J=3.2 Hz, 1H), 7.19 (dd, J=8.5, 1.9 Hz, 1H), 7.60 (d, J=8.3 Hz, 1H).

### Example 189

### cyclopropylmethyl ({2-[5-(5-chloro-1H-indol-1-yl)-1,3-dimethyl-1H-pyrazol-4-yl]ethyl}sulfonyl)carbamate

To a solution of cyclopropylmethanol (78.4 mg) in N,N-dimethylformamide (9 mL) was added N,N′-carbonyldiimidazole (191 mg), and the mixture was stirred at 60° C. for 1 hr. 2-[5-(5-Chloro-1H-indol-1-yl)-1,3-dimethyl-1H-pyrazol-4-yl]ethanesulfonamide obtained in Reference Example 179 (320 mg), 1,8-diazabicyclo[5.4.0]undec-7-ene (207 mg) and 4-dimethylaminopyridine (166 mg) were added to this reaction mixture, and the mixture was stirred at 60° C. for 20 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 45:55, v/v), and crystallized from hexane-ethyl acetate to give the title compound (182 mg, yield 45%) as colorless crystals. melting point 169.8-170.4° C.

¹H-NMR (300 MHz, CDCl₃) δ:0.24-0.30 (m, 2H), 0.54-0.62 (m, 2H), 0.98-1.11 (m, 1H), 2.32 (s, 3H), 2.66-2.87 (m, 2H), 3.32 (t, J=8.0 Hz, 2H), 3.47 (s, 3H), 3.87 (d, J=7.3 Hz, 2H), 6.70 (d, J=3.2 Hz, 1H), 6.92 (d, J=8.7 Hz, 1H), 7.12 (d, J=3.2 Hz, 1H), 7.18-7.23 (m, 1H), 7.25-7.27 (m, 1H), 7.66 (d, J=2.1 Hz, 1H).

### Example 190

### butyl ({2-[5-(5-chloro-1H-pyrrolo[2,3-b]pyridin-1-yl)-1,3-dimethyl-1H-pyrazol-4-yl]ethyl}sulfonyl)carbamate

By a method similar to that in Example 186, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184, butanol and N,N′-carbonyldiimidazole.

¹H-NMR (300 MHz, CDCl₃) δ:0.93 (t, J=7.3 Hz, 3H), 1.29-1.43 (m, 2H), 1.53-1.64 (m, 2H), 2.31 (s, 3H), 2.78-2.87 (m, 2H), 3.11-3.20 (m, 1H), 3.36 (s, 3H), 3.90-4.04 (m, 2H), 4.06-4.18 (m, 1H), 6.70 (d, J=3.8 Hz, 1H), 7.16 (d, J=3.6 Hz, 1H), 8.04 (d, J=2.1 Hz, 1H), 8.28 (d, J=2.1 Hz, 1H), 10.57 (br s, 1H).

### Example 191

### butyl ({(E)-2-[5-(5-chloro-1H-indol-1-yl)-1,3-dimethyl-1H-pyrazol-4-yl]vinyl}sulfonyl)carbamate

By a method similar to that in Example 186, the title compound was obtained from (E)-2-[5-(5-chloro-1H-indol-1-yl)-1,3-dimethyl-1H-pyrazol-4-yl]ethylenesulfonamide obtained in Reference Example 178, butanol and N,N′-carbonyldiimidazole.

¹H-NMR (300 MHz, CDCl₃) δ:0.91 (t, J=7.4 Hz, 3H), 1.27-1.40 (m, 2H), 1.50-1.62 (m, 2H), 2.45 (s, 3H), 3.54 (s, 3H), 4.00-4.15 (m, 2H), 5.86 (d, J=15.5 Hz, 1H), 6.78 (d, J=3.0 Hz, 1H), 6.91 (d, J=8.7 Hz, 1H), 7.11 (d, J=3.4 Hz, 1H), 7.20 (dd, J=8.7, 1.9 Hz, 1H), 7.33 (d, J=15.5 Hz, 1H), 7.69 (d, J=1.9 Hz, 1H).

### Example 192

### butyl ({2-[5-(5-fluoro-1H-indol-1-yl)-1,3-dimethyl-1H-pyrazol-4-yl]ethyl}sulfonyl)carbamate

By a method similar to that in Example 186, the title compound was obtained from 2-[5-(5-fluoro-1H-indol-1-yl)-1,3-dimethyl-1H-pyrazol-4-yl]ethanesulfonamide obtained in Reference Example 186, butanol and N,N′-carbonyldiimidazole.

¹H-NMR (300 MHz, CDCl₃) δ:0.89-0.96 (m, 3H), 1.25-1.40 (m, 2H), 1.51-1.62 (m, 2H), 2.31 (s, 3H), 2.66-2.87 (m, 2H), 3.30 (t, J=8.0 Hz, 2H), 3.47 (s, 3H), 4.06 (t, J=6.6 Hz, 2H), 6.71 (d, J=2.7 Hz, 1H), 6.88-6.94 (m, 1H), 6.95-7.03 (m, 1H), 7.13 (d, J=3.4 Hz, 1H), 7.34 (dd, J=9.1, 2.3 Hz, 1H).

### Example 193

### propyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, propanol and N,N′-carbonyldiimidazole.

¹H-NMR (300 MHz, CDCl₃) δ:0.91 (t, J=7.4 Hz, 3H), 1.53-1.67 (m, 2H), 2.31 (s, 3H), 2.65-2.87 (m, 2H), 3.30 (t, J=8.0 Hz, 2H), 3.46 (s, 3H), 4.01 (t, J=6.7 Hz, 2H), 6.70 (d, J=3.2 Hz, 1H), 6.92 (d, J=8.7 Hz, 1H), 7.12 (d, J=3.4 Hz, 1H), 7.20 (dd, J=8.7, 1.9 Hz, 1H), 7.67 (d, J=1.9 Hz, 1H).

### Example 194

### butyl ({2-[1,3-dimethyl-5-(1H-pyrrolo[2,3-b]pyridin-1-yl)-1H-pyrazol-4-yl]ethyl}sulfonyl)carbamate

By a method similar to that in Example 186, the title compound was obtained from 2-[1,3-dimethyl-5-(1H-pyrrolo[2,3-b]pyridin-1-yl)-1H-pyrazol-4-yl]ethanesulfonamide obtained in Reference Example 188, butanol and N,N′-carbonyldiimidazole.

¹H-NMR (300 MHz, CDCl₃) δ:0.92 (t, J=7.4 Hz, 3H), 1.29-1.43 (m, 2H), 1.53-1.64 (m, 2H), 2.32 (s, 3H), 2.79-2.87 (m, 2H), 3.08-3.18 (m, 1H), 3.34 (s, 3H), 3.87-3.98 (m, 1H), 4.00-4.17 (m, 2H), 6.74 (d, J=3.8 Hz, 1H), 7.11 (d, J=3.8 Hz, 1H), 7.22-7.28 (m, 1H), 8.07 (dd, J=8.0, 1.5 Hz, 1H), 8.31 (dd, J=4.9, 1.5 Hz, 1H), 11.71 (br s, 1H).

### Example 195

### isobutyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, 2-methylpropan-1-ol and N,N′-carbonyldiimidazole.

¹H-NMR (300 MHz, CDCl₃) δ:0.89 (d, J=6.8 Hz, 6H), 1.79-1.92 (m, 1H), 2.31 (s, 3H), 2.65-2.88 (m, 2H), 3.30 (t, J=7.9 Hz, 2H), 3.46 (s, 3H), 3.83 (d, J=6.8 Hz, 2H), 6.70 (dd, J=3.4, 0.8 Hz, 1H), 6.92 (d, J=8.9 Hz, 1H), 7.11 (d, J=3.2 Hz, 1H), 7.20 (dd, J=8.8, 2.0 Hz, 1H), 7.67 (d, J=1.7 Hz, 1H).

### Example 196

### butyl ({2-[5-(6-methoxy-1H-indol-1-yl)-1,3-dimethyl-1H-pyrazol-4-yl]ethyl}sulfonyl)carbamate

By a method similar to that in Example 186, the title compound was obtained from 2-[5-(6-methoxy-1H-indol-1-yl)-1,3-dimethyl-1H-pyrazol-4-yl]ethanesulfonamide obtained in Reference Example 190, butanol and N,N′-carbonyldiimidazole.

¹H-NMR (300 MHz, CDCl₃) δ:0.92 (t, J=7.3 Hz, 3H), 1.25-1.41 (m, 2H), 1.50-1.62 (m, 2H), 2.32 (s, 3H), 2.67-2.89 (m, 2H), 3.32 (t, J=7.8 Hz, 2H), 3.48 (s, 3H), 3.78 (s, 3H), 4.05 (t, J=6.7 Hz, 2H), 6.43 (d, J=2.3 Hz, 1H), 6.64-6.68 (m, 1H), 6.86 (dd, J=8.7, 2.3 Hz, 1H), 6.96 (d, J=3.2 Hz, 1H), 7.55 (d, J=8.7 Hz, 1H).

### Example 197

### butyl ({2-[5-(5-chloro-1H-pyrrolo[2,3-b]pyridin-1-yl)-1-methyl-3-(trifluoromethyl)-1H-pyrazol-4-yl]ethyl}sulfonyl)carbamate

To a solution of butanol (109 mg) in N,N-dimethylformamide (10 mL) was added N,N′-carbonyldiimidazole (254 mg), and the mixture was stirred at 60° C. for 1 hr. 2-[5-(5-Chloro-1H-pyrrolo[2,3-b]pyridin-1-yl)-1-methyl-3-(trifluoromethyl)-1H-pyrazol-4-yl]ethanesulfonamide obtained in Reference Example 193 (400 mg), 1,8-diazabicyclo[5.4.0]undec-7-ene (209 mg) and 4-dimethylaminopyridine (168 mg) were added to this reaction mixture, and the mixture was stirred at 60° C. for 22 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 60:40, v/v), and crystallized from hexane-ethyl acetate to give the title compound (150 mg, yield 30%) as colorless crystals. melting point 136.1-137.3° C.

¹H-NMR (300 MHz, CDCl₃) δ:0.95 (t, J=7.2 Hz, 3H), 1.30-1.44 (m, 2H), 1.54-1.66 (m, 2H), 2.87-3.08 (m, 2H), 3.21-3.31 (m, 1H), 3.54 (s, 3H), 3.83-3.96 (m, 1H), 4.08 (t, J=6.6 Hz, 2H), 6.76 (d, J=3.4 Hz, 1H), 7.21 (d, J=3.8 Hz, 1H), 8.06 (d, J=2.3 Hz, 1H), 8.30 (d, J=2.3 Hz, 1H), 9.32 (br s, 1H).

### Example 198

### butyl [(2-{1,3-dimethyl-5-[6-(trifluoromethyl)-1H-indol-1-yl]-1H-pyrazol-4-yl}ethyl)sulfonyl]carbamate

By a method similar to that in Example 186, the title compound was obtained from 2-{1,3-dimethyl-5-[6-(trifluoromethyl)-1H-indol-1-yl]-1H-pyrazol-4-yl}ethanesulfonamide obtained in Reference Example 195, butanol and N,N′-carbonyldiimidazole.

¹H-NMR (300 MHz, CDCl₃) δ:0.92 (t, J=7.3 Hz, 3H), 1.25-1.40 (m, 2H), 1.51-1.62 (m, 2H), 2.34 (s, 3H), 2.64-2.76 (m, 1H), 2.79-2.91 (m, 1H), 3.33 (t, J=7.9 Hz, 2H), 3.47 (s, 3H), 4.06 (t, J=6.7 Hz, 2H), 6.83 (dd, J=3.3, 0.8 Hz, 1H), 7.24-7.28 (m, 2H), 7.47 (dd, J=8.3, 0.9 Hz, 1H), 7.80 (d, J=8.3 Hz, 1H).

### Example 199

### N-({(E)-2-[5-(5-chloro-1H-indol-1-yl)-1,3-dimethyl-1H-pyrazol-4-yl]vinyl}sulfonyl)hexanamide

A mixture of (E)-2-[5-(5-chloro-1H-indol-1-yl)-1,3-dimethyl-1H-pyrazol-4-yl]ethylenesulfonamide obtained in Reference Example 178 (213 mg), hexanoic acid (74.1 mg), 2-methyl-6-nitrobenzoic anhydride (251 mg), triethylamine (184 mg), 4-dimethylaminopyridine (74.3 mg) and acetonitrile (6 mL) was stirred at room temperature for 24 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 50:50, v/v) to give the title compound (198 mg, yield 73%) as a colorless amorphous solid.

¹H-NMR (300 MHz, CDCl₃) δ:0.89 (t, J=6.9 Hz, 3H), 1.20-1.35 (m, 4H), 1.49-1.62 (m, 2H), 2.14 (t, J=7.5 Hz, 2H), 2.44 (s, 3H), 3.55 (s, 3H), 5.79 (d, J=15.6 Hz, 1H), 6.77 (d, J=3.2 Hz, 1H), 6.90 (d, J=8.7 Hz, 1H), 7.11 (d, J=3.2 Hz, 1H), 7.19 (dd, J=8.7, 1.9 Hz, 1H), 7.35 (d, J=15.6 Hz, 1H), 7.61 (br s, 1H), 7.69 (d, J=1.9 Hz, 1H).

### Example 200

### N-({2-[5-(5-chloro-1H-indol-1-yl)-1,3-dimethyl-1H-pyrazol-4-yl]ethyl}sulfonyl)hexanamide

By a method similar to that in Example 199, the title compound was obtained from 2-[5-(5-chloro-1H-indol-1-yl)-1,3-dimethyl-1H-pyrazol-4-yl]ethanesulfonamide obtained in Reference Example 179 and hexanoic acid.

¹H-NMR (300 MHz, CDCl₃) δ:0.89 (t, J=6.9 Hz, 3H), 1.17-1.34 (m, 4H), 1.42-1.55 (m, 2H), 1.98-2.06 (m, 2H), 2.30 (s, 3H), 2.58-2.70 (m, 1H), 2.71-2.84 (m, 1H), 3.33 (t, J=7.7 Hz, 2H), 3.46 (s, 3H), 6.70 (d, J=3.2 Hz, 1H), 6.91 (d, J=8.7 Hz, 1H), 7.13 (d, J=3.2 Hz, 1H), 7.20 (dd, J=8.7, 2.1 Hz, 1H), 7.66 (d, J=1.9 Hz, 1H).

### Example 201

### N-({2-[1,3-dimethyl-5-(1H-pyrrolo[2,3-b]pyridin-1-yl)-1H-pyrazol-4-yl]ethyl}sulfonyl)hexanamide

By a method similar to that in Example 199, the title compound was obtained from 2-[1,3-dimethyl-5-(1H-pyrrolo[2,3-b]pyridin-1-yl)-1H-pyrazol-4-yl]ethanesulfonamide obtained in Reference Example 188 and hexanoic acid.

¹H-NMR (300 MHz, CDCl₃) δ:0.87-0.93 (m, 3H), 1.23-1.39 (m, 4H), 1.53-1.66 (m, 2H), 1.81-1.92 (m, 1H), 2.03-2.16 (m, 1H), 2.29 (s, 3H), 2.81-2.87 (m, 2H), 3.07-3.16 (m, 1H), 3.36 (s, 3H), 3.98-4.14 (m, 1H), 6.76 (d, J=3.6 Hz, 1H), 7.13 (d, J=3.6 Hz, 1H), 7.25-7.30 (m, 1H), 8.10 (dd, J=7.9, 1.5 Hz, 1H), 8.29 (dd, J=4.9, 1.5 Hz, 1H), 11.49 (br s, 1H).

### Example 202

### N-({2-[5-(5-chloro-1H-pyrrolo[2,3-b]pyridin-1-yl)-1,3-dimethyl-1H-pyrazol-4-yl]ethyl}sulfonyl)hexanamide

By a method similar to that in Example 199, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184 and hexanoic acid.

¹H-NMR (300 MHz, CDCl₃) δ:0.91 (t, J=6.8 Hz, 3H), 1.23-1.39 (m, 4H), 1.52-1.65 (m, 2H), 1.80-1.92 (m, 1H), 2.00-2.13 (m, 1H), 2.28 (s, 3H), 2.80-2.87 (m, 2H), 3.09-3.19 (m, 1H), 3.38 (s, 3H), 3.95-4.12 (m, 1H), 6.72 (d, J=3.6 Hz, 1H), 7.18 (d, J=3.6 Hz, 1H), 8.07 (d, J=2.1 Hz, 1H), 8.26 (d, J=2.3 Hz, 1H), 10.61 (br s, 1H).

### Example 203

### butyl {(2E)-3-[5-(5-chloro-1H-indol-1-yl)-1,3-dimethyl-1H-pyrazol-4-yl]prop-2-enoyl}sulfamate

A mixture of (2E)-3-[5-(5-chloro-1H-indol-1-yl)-1,3-dimethyl-1H-pyrazol-4-yl]acrylic acid obtained in Reference Example 38 (342 mg), 2-methyl-6-nitrobenzoic anhydride (448 mg), butyl sulfamate obtained in Reference Example 196 (174 mg), triethylamine (329 mg), 4-dimethylaminopyridine (132 mg) and acetonitrile (10 mL) was stirred at room temperature for 16 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 40:60, v/v) to give the title compound (187 mg, yield 38%) as a colorless amorphous solid.

¹H-NMR (300 MHz, CDCl₃) δ:0.90 (t, J=7.4 Hz, 3H), 1.28-1.43 (m, 2H), 1.59-1.70 (m, 2H), 2.44 (s, 3H), 3.52 (s, 3H), 4.26 (t, J=6.6 Hz, 2H), 5.56 (d, J=15.5 Hz, 1H), 6.78 (d, J=3.4 Hz, 1H), 6.90 (d, J=8.7 Hz, 1H), 7.11 (d, J=3.4 Hz, 1H), 7.21 (dd, J=8.7, 1.9 Hz, 1H), 7.47 (d, J=15.9 Hz, 1H), 7.70 (d, J=1.5 Hz, 1H).

### Example 204

### butyl {(2E)-3-[5-(3-chloro-1H-pyrrolo[2,3-b]pyridin-1-yl)-1,3-dimethyl-1H-pyrazol-4-yl]prop-2-enoyl}sulfamate

By a method similar to that in Example 203, the title compound was obtained from (2E)-3-[5-(3-chloro-1H-pyrrolo[2,3-b]pyridin-1-yl)-1,3-dimethyl-1H-pyrazol-4-yl]acrylic acid obtained in Reference Example 133 and butyl sulfamate obtained in Reference Example 196.

¹H-NMR (300 MHz, DMSO-d₆) δ:0.82 (t, J=7.2 Hz, 3H), 1.20-1.33 (m, 2H), 1.50-1.63 (m, 2H), 2.39 (s, 3H), 3.51 (s, 3H), 4.16 (t, J=5.9 Hz, 2H), 6.06 (d, J=16.3 Hz, 1H), 7.08 (d, J=15.9 Hz, 1H), 7.37-7.45 (m, 1H), 8.07 (s, 1H), 8.18 (d, J=7.2 Hz, 1H), 8.38 (d, J=3.0 Hz, 1H), 12.07 (br s, 1H).

### Example 205

### butyl {(2E)-3-[5-(5-cyano-1H-indol-1-yl)-1,3-dimethyl-1H-pyrazol-4-yl]prop-2-enoyl}sulfamate

By a method similar to that in Example 203, the title compound was obtained from (2E)-3-[5-(5-cyano-1H-indol-1-yl)-1,3-dimethyl-1H-pyrazol-4-yl]acrylic acid obtained in Reference Example 81 and butyl sulfamate obtained in Reference Example 196.

¹H-NMR (300 MHz, CDCl₃) δ:0.90 (t, J=7.3 Hz, 3H), 1.31-1.43 (m, 2H), 1.59-1.71 (m, 2H), 2.46 (s, 3H), 3.52 (s, 3H), 4.28 (t, J=6.6 Hz, 2H), 5.68 (d, J=15.8 Hz, 1H), 6.90-6.94 (m, 1H), 7.07 (d, J=8.5 Hz, 1H), 7.24 (d, J=3.4 Hz, 1H), 7.42 (d, J=15.6 Hz, 1H), 7.47-7.52 (m, 1H), 8.07 (d, J=0.8 Hz, 1H), 8.26 (br s, 1H).

### Example 206

### butyl {(2E)-3-[5-(5-fluoro-1H-indol-1-yl)-1,3-dimethyl-1H-pyrazol-4-yl]prop-2-enoyl}sulfamate

By a method similar to that in Example 203, the title compound was obtained from (2E)-3-[5-(5-fluoro-1H-indol-1-yl)-1,3-dimethyl-1H-pyrazol-4-yl]acrylic acid obtained in Reference Example 21 and butyl sulfamate obtained in Reference Example 196.

¹H-NMR (300 MHz, CDCl₃) δ:0.90 (t, J=7.3 Hz, 3H), 1.29-1.43 (m, 2H), 1.59-1.71 (m, 2H), 2.44 (s, 3H), 3.52 (s, 3H), 4.27 (t, J=6.5 Hz, 2H), 5.56 (d, J=15.8 Hz, 1H), 6.77-6.80 (m, 1H), 6.87-6.93 (m, 1H), 6.95-7.04 (m, 1H), 7.12 (d, J=3.4 Hz, 1H), 7.37 (dd, J=9.0, 2.4 Hz, 1H), 7.47 (d, J=15.8 Hz, 1H), 7.93 (br s, 1H).

### Example 207

### N-[({[5-(5-chloro-1H-indol-1-yl)-1,3-dimethyl-1H-pyrazol-4-yl]methyl}amino)sulfonyl]hexanamide

A mixture of N-{[5-(5-chloro-1H-indol-1-yl)-1,3-dimethyl-1H-pyrazol-4-yl]methyl}sulfamide obtained in Reference Example 197 (63.3 mg), 2-methyl-6-nitrobenzoic anhydride (70.4 mg), hexanoic acid (19.7 mg), triethylamine (51.7 mg), 4-dimethylaminopyridine (20.8 mg) and acetonitrile (2 mL) was stirred at room temperature for 48 hr. A saturated aqueous ammonium chloride solution (5 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 50:50, v/v) to give the title compound (53.3 mg, yield 66%) as colorless crystals.

¹H-NMR (300 MHz, CDCl₃) δ:0.88 (t, J=6.8 Hz, 3H), 1.15-1.33 (m, 4H), 1.39-1.51 (m, 2H), 1.96-2.07 (m, 2H), 2.35 (s, 3H), 3.47 (s, 3H), 3.63-3.94 (m, 2H), 5.15 (br s, 1H), 6.69 (d, J=2.7 Hz, 1H), 6.92 (d, J=8.7 Hz, 1H), 7.15-7.24 (m, 2H), 7.62-7.78 (m, 2H).

### Example 208

### 2-[5-(5-chloro-1H-indol-1-yl)-1,3-dimethyl-1H-pyrazol-4-yl]-N-{[(2,2,2-trifluoroethyl)amino]carbonyl}ethanesulfonamide

To a solution of 2,2,2-trifluoroethanamine (134 mg) in N,N-dimethylformamide (11 mL) was added N,N′-carbonyldiimidazole (238 mg), and the mixture was stirred at 60° C. for 1 hr. 2-[5-(5-Chloro-1H-indol-1-yl)-1,3-dimethyl-1H-pyrazol-4-yl]ethanesulfonamide obtained in Reference Example 179 (400 mg), 1,8-diazabicyclo[5.4.0]undec-7-ene (241 mg) and 4-dimethylaminopyridine (193 mg) were added to the reaction mixture, and the mixture was stirred at 60° C. for 16 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 30:70, v/v), and crystallized from hexane-ethyl acetate to give the title compound (207 mg, yield 38%) as colorless crystals.

¹H-NMR (300 MHz, DMSO-d₆) δ:2.18 (s, 3H), 2.37-2.47 (m, 1H), 2.54-2.65 (m, 1H), 3.23-3.30 (m, 2H), 3.37 (s, 3H), 3.73-3.88 (m, 2H), 6.77 (d, J=2.6 Hz, 1H), 6.98-7.07 (m, 2H), 7.20 (dd, J=8.8, 2.0 Hz, 1H), 7.58 (d, J=3.4 Hz, 1H), 7.75 (d, J=1.9 Hz, 1H), 10.48 (br s, 1H).

### Example 209

### N-[(butylamino)carbonyl]-2-[5-(6-chloro-1H-indol-1-yl)-1,3-dimethyl-1H-pyrazol-4-yl]ethanesulfonamide

By a method similar to that in Example 208, the title compound was obtained from 2-[5-(6-chloro-1H-indol-1-yl)-1,3-dimethyl-1H-pyrazol-4-yl]ethanesulfonamide obtained in Reference Example 181, butylamine and N,N′-carbonyldiimidazole.

¹H-NMR (300 MHz, CDCl₃) δ:0.87-0.95 (m, 3H), 1.23-1.34 (m, 2H), 1.35-1.49 (m, 2H), 2.31 (s, 3H), 2.68-2.90 (m, 2H), 2.96-3.17 (m, 4H), 3.48 (s, 3H), 6.17 (br s, 1H), 6.73 (dd, J=3.3, 0.8 Hz, 1H), 6.97 (d, J=0.8 Hz, 1H), 7.08 (d, J=3.4 Hz, 1H), 7.19 (dd, J=8.5, 1.7 Hz, 1H), 7.59-7.63 (m, 1H).

### Example 210

### N-[(butylamino)carbonyl]-2-[5-(5-chloro-1H-indol-1-yl)-1,3-dimethyl-1H-pyrazol-4-yl]ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, butylamine and N,N′-carbonyldiimidazole.

¹H-NMR (300 MHz, CDCl₃) δ:0.92 (t, J=7.2 Hz, 3H), 1.22-1.35 (m, 2H), 1.36-1.48 (m, 2H), 2.30 (s, 3H), 2.69-2.89 (m, 2H), 2.96-3.17 (m, 4H), 3.47 (s, 3H), 6.13 (br s, 1H), 6.71 (d, J=3.2 Hz, 1H), 6.91 (d, J=8.7 Hz, 1H), 7.11 (1H, d, J=3.4 Hz), 7.20 (dd, J=8.8, 1.8 Hz, 1H), 7.67 (d, J=1.5 Hz, 1H).

### Example 211

### 2-[5-(5-chloro-1H-indol-1-yl)-1,3-dimethyl-1H-pyrazol-4-yl]-N-{[(cyclopropylmethyl)amino]carbonyl}ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, cyclopropylmethylamine and N,N′-carbonyldiimidazole.

¹H-NMR (300 MHz, CDCl₃) δ:0.12-0.19 (m, 2H), 0.44-0.52 (m, 2H), 0.79-0.91 (m, 1H), 2.30 (s, 3H), 2.73-2.89 (m, 2H), 2.93-3.10 (m, 4H), 3.47 (s, 3H), 6.21 (br s, 1H), 6.70 (d, J=3.2 Hz, 1H), 6.90 (d, J=8.7 Hz, 1H), 7.12 (d, J=3.2 Hz, 1H), 7.19 (dd, J=8.7, 2.1 Hz, 1H), 7.66 (d, J=1.9 Hz, 1H).

### Example 212

### (E)-N-[(butylamino)carbonyl]-2-[5-(5-chloro-1H-indol-1-yl)-1,3-dimethyl-1H-pyrazol-4-yl]ethylenesulfonamide

By a method similar to that in Example 208, the title compound was obtained from (E)-2-[5-(5-chloro-1H-indol-1-yl)-1,3-dimethyl-1H-pyrazol-4-yl]ethylenesulfonamide obtained in Reference Example 178, butylamine and N,N′-carbonyldiimidazole.

¹H-NMR (300 MHz, CDCl₃) δ:0.89 (t, J=7.2 Hz, 3H), 1.22-1.31 (m, 2H), 1.33-1.43 (m, 2H), 2.43 (s, 3H), 3.03-3.21 (m, 2 H), 3.54 (s, 3H), 5.85 (d, J=15.6 Hz, 1H), 6.18 (br s, 1H), 6.78 (d, J=3.2 Hz, 1H), 6.90 (d, J=8.7 Hz, 1H), 7.08 (d, J=3.2 Hz, 1H), 7.16 (d, J=15.4 Hz, 1H), 7.23 (dd, J=8.9, 1.9 Hz, 1H), 7.69 (d, J=1.9 Hz, 1H).

### Example 213

### N-[(butylamino)carbonyl]-2-[1,3-dimethyl-5-(1H-pyrrolo[2,3-b]pyridin-1-yl)-1H-pyrazol-4-yl]ethanesulfonamide

By a method similar to that in Example 208, the title compound was obtained from 2-[1,3-dimethyl-5-(1H-pyrrolo[2,3-b]pyridin-1-yl)-1H-pyrazol-4-yl]ethanesulfonamide obtained in Reference Example 188, butylamine and N,N′-carbonyldiimidazole.

¹H-NMR (300 MHz, CDCl₃) δ:0.93 (t, J=7.2 Hz, 3H), 1.25-1.37 (m, 2H), 1.39-1.51 (m, 2H), 2.33 (s, 3H), 2.79-2.89 (m, 2H), 2.95-3.07 (m, 1H), 3.11-3.25 (m, 2H), 3.38 (s, 3H), 3.92 (br s, 1H), 5.39-5.49 (m, 1H), 6.76 (d, J=3.6 Hz, 1H), 7.15 (d, J=3.6 Hz, 1H), 7.24-7.30 (m, 1H), 8.10 (dd, J=7.8, 1.4 Hz, 1H), 8.29-8.33 (m, 1H).

### Example 214

### N-[(butylamino)carbonyl]-2-[5-(5-chloro-1H-pyrrolo[2,3-b]pyridin-1-yl)-1,3-dimethyl-1H-pyrazol-4-yl]ethanesulfonamide

By a method similar to that in Example 208, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184, butylamine and N,N′-carbonyldiimidazole.

¹H-NMR (300 MHz, CDCl₃) δ:0.89-0.96 (m, 3H), 1.25-1.37 (m, 2H), 1.40-1.52 (m, 2H), 2.31 (s, 3H), 2.77-2.88 (m, 2H), 3.04-3.25 (m, 3H), 3.42 (s, 3H), 3.75 (br s, 1H), 5.54 (br s, 1H), 6.71 (d, J=3.6 Hz, 1H), 7.20 (d, J=3.6 Hz, 1H), 8.05 (d, J=2.1 Hz, 1H), 8.27 (d, J=2.1 Hz, 1H).

### Example 215

### N-[(butylamino)carbonyl]-2-[5-(5-chloro-1H-pyrrolo[2,3-b]pyridin-1-yl)-1-methyl-3-(trifluoromethyl)-1H-pyrazol-4-yl]ethanesulfonamide

By a method similar to that in Example 208, the title compound was obtained from 2-[5-(5-chloro-1H-pyrrolo[2,3-b]pyridin-1-yl)-1-methyl-3-(trifluoromethyl)-1H-pyrazol-4-yl]ethanesulfonamide obtained in Reference Example 193, butylamine and N,N′-carbonyldiimidazole.

¹H-NMR (300 MHz, CDCl₃) δ:0.92 (t, J=7.2 Hz, 3H), 1.24-1.39 (m, 2H), 1.41-1.52 (m, 2H), 2.84-3.05 (m, 2H), 3.10-3.21 (m, 2H), 3.30 (br s, 1H), 3.49-3.63 (m, 4H), 6.05 (br s, 1H), 6.76 (d, J=3.8 Hz, 1H), 7.23 (d, J=3.8 Hz, 1H), 8.05 (d, J=2.3 Hz, 1H), 8.28 (d, J=2.3 Hz, 1H).

### Example 216

### N-[(butylamino)carbonyl]-2-[5-(6-methoxy-1H-indol-1-yl)-1,3-dimethyl-1H-pyrazol-4-yl]ethanesulfonamide

By a method similar to that in Example 208, the title compound was obtained from 2-[5-(6-methoxy-1H-indol-1-yl)-1,3-dimethyl-1H-pyrazol-4-yl]ethanesulfonamide obtained in Reference Example 190, butylamine and N,N′-carbonyldiimidazole.

¹H-NMR (300 MHz, CDCl₃) δ:0.88-0.94 (m, 3H), 1.24-1.34 (m, 2H), 1.35-1.48 (m, 2H), 2.31 (s, 3H), 2.74-2.89 (m, 2H), 3.00-3.16 (m, 4H), 3.50 (s, 3H), 3.78 (s, 3H), 6.07 (br s, 1H), 6.43 (d, J=2.1 Hz, 1H), 6.67 (dd, J=3.2, 0.8 Hz, 1H), 6.87 (dd, J=8.7, 2.3 Hz, 1H), 6.96 (d, J=3.2 Hz, 1H), 7.56 (d, J=8.5 Hz, 1H).

### Example 217

### 2-[5-(5-chloro-1H-indol-1-yl)-1,3-dimethyl-1H-pyrazol-4-yl]-N-[(propylamino)carbonyl]ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, propylamine and N,N′-carbonyldiimidazole.

¹H-NMR (300 MHz, CDCl₃) δ:0.88 (t, J=7.4 Hz, 3H), 1.38-1.51 (m, 2H), 2.30 (s, 3H), 2.68-2.88 (m, 2H), 2.99-3.12 (m, 4H), 3.48 (s, 3H), 6.15 (br s, 1H), 6.70 (d, J=3.0 Hz, 1H), 6.91 (d, J=8.7 Hz, 1H), 7.11 (d, J=3.0 Hz, 1H), 7.20 (dd, J=8.7, 1.9 Hz, 1H), 7.67 (d, J=1.9 Hz, 1H).

### Example 218

### N-[(butylamino)carbonyl]-2-[5-(5-fluoro-1H-indol-1-yl)-1,3-dimethyl-1H-pyrazol-4-yl]ethanesulfonamide

By a method similar to that in Example 208, the title compound was obtained from 2-[5-(5-fluoro-1H-indol-1-yl)-1,3-dimethyl-1H-pyrazol-4-yl]ethanesulfonamide obtained in Reference Example 186, butylamine and N,N′-carbonyldiimidazole.

¹H-NMR (300 MHz, CDCl₃) δ:0.87-0.95 (m, 3H), 1.23-1.48 (m, 4H), 2.30 (s, 3H), 2.68-2.89 (m, 2H), 2.99-3.16 (m, 4H), 3.48 (s, 3H), 6.13 (br s, 1H), 6.72 (d, J=2.7 Hz, 1H), 6.87-6.93 (m, 1H), 6.94-7.03 (m, 1H), 7.12 (d, J=3.4 Hz, 1H), 7.34 (dd, J=9.1, 2.3 Hz, 1H).

### Example 219

### 2-[5-(5-chloro-1H-indol-1-yl)-1,3-dimethyl-1H-pyrazol-4-yl]-N-[(isobutylamino)carbonyl]ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, isobutylamine and N,N′-carbonyldiimidazole.

¹H-NMR (300 MHz, CDCl₃) δ:0.86 (d, J=6.6 Hz, 6H), 1.67 (dd, J=13.6, 6.6 Hz, 1H), 2.30 (s, 3H), 2.69-2.89 (m, 2H), 2.95 (t, J=6.4 Hz, 2H), 2.99-3.11 (m, 2H), 3.47 (s, 3H), 6.22 (br s, 1H), 6.70 (d, J=3.2 Hz, 1H), 6.90 (d, J=8.9 Hz, 1H), 7.11 (d, J=3.0 Hz, 1H), 7.20 (dd, J=8.7, 1.3 Hz, 1H), 7.67 (d, J=1.3 Hz, 1H).

### Example 220

### N-[(butylamino)carbonyl]-2-{1,3-dimethyl-5-[6-(trifluoromethyl)-1H-indol-1-yl]-1H-pyrazol-4-yl}ethanesulfonamide

By a method similar to that in Example 208, the title compound was obtained from 2-{1,3-dimethyl-5-[6-(trifluoromethyl)-1H-indol-1-yl]-1H-pyrazol-4-yl}ethanesulfonamide obtained in Reference Example 195, butylamine and N,N′-carbonyldiimidazole.

¹H-NMR (300 MHz, CDCl₃) δ:0.87-0.94 (m, 3H), 1.22-1.34 (m, 2H), 1.36-1.47 (m, 2H), 2.33 (s, 3H), 2.66-2.78 (m, 1H), 2.79-2.91 (m, 1H), 2.98-3.17 (m, 4H), 3.48 (s, 3H), 6.16 (br s, 1H), 6.83 (dd, J=3.4, 0.8 Hz, 1H), 7.23-7.28 (m, 2H), 7.47 (dd, J=8.3, 1.1 Hz, 1H), 7.80 (d, J=8.3 Hz, 1H).

### Example 221

### propyl ({2-[5-(6-chloro-1H-indol-1-yl)-1,3-dimethyl-1H-pyrazol-4-yl]ethyl}sulfonyl)carbamate

By a method similar to that in Example 186, the title compound was obtained from 2-[5-(6-chloro-1H-indol-1-yl)-1,3-dimethyl-1H-pyrazol-4-yl]ethanesulfonamide obtained in Reference Example 181, propanol and N,N′-carbonyldiimidazole.

¹H-NMR (300 MHz, CDCl₃) δ:0.91 (t, J=7.4 Hz, 3H), 1.54-1.67 (m, 2H), 2.31 (s, 3H), 2.65-2.90 (m, 2H), 3.32 (t, J=7.8 Hz, 2H), 3.47 (s, 3H), 4.02 (t, J=6.8 Hz, 2H), 6.73 (dd, J=3.3, 0.8 Hz, 1H), 6.97-6.99 (m, 1H), 7.09 (d, J=3.4 Hz, 1H), 7.19 (dd, J=8.4, 1.8 Hz, 1H), 7.60 (d, J=8.3 Hz, 1H).

### Example 222

### 2-[5-(5-chloro-1H-indol-1-yl)-1,3-dimethyl-1H-pyrazol-4-yl]-N-{[(3-methylbutyl)amino]carbonyl}ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, 3-methylbutan-1-amine and N,N′-carbonyldiimidazole.

¹H-NMR (300 MHz, CDCl₃) δ:0.90 (d, J=6.6 Hz, 6H), 1.33 (q, J=7.0 Hz, 2H), 1.50-1.63 (m, 1H), 2.30 (s, 3H), 2.70-2.89 (m, 2H), 2.94-3.08 (m, 2H), 3.09-3.20 (m, 2H), 3.45-3.50 (m, 3H), 6.09 (br s, 1H), 6.71 (d, J=3.4 Hz, 1H), 6.91 (d, J=8.7 Hz, 1H), 7.12 (d, J=3.4 Hz, 1H), 7.20 (dd, J=8.8, 1.8 Hz, 1H), 7.67 (d, J=1.9 Hz, 1H).

### Example 223

### benzyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, benzyl alcohol and N,N′-carbonyldiimidazole.

¹H-NMR (300 MHz, CDCl₃) δ:2.26 (s, 3H), 2.62-2.86 (m, 2H), 3.30 (t, J=7.9 Hz, 2H), 3.45 (s, 3H), 5.07 (s, 2H), 6.66 (dd, J=3.3, 0.8 Hz, 1H), 6.90 (d, J=8.7 Hz, 1H), 7.08 (d, J=3.2 Hz, 1H), 7.19 (dd, J=8.7, 1.9 Hz, 1H), 7.28-7.33 (m, 2H), 7.36-7.40 (m, 3H), 7.64 (d, J=1.7 Hz, 1H).

### Example 224

### 2,2,2-trifluoroethyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, 2,2,2-trifluoroethanol and N,N′-carbonyldiimidazole.

¹H-NMR (300 MHz, CDCl₃) δ:2.29 (s, 3H), 2.67-2.84 (m, 2H), 3.26 (t, J=7.8 Hz, 2H), 3.47 (s, 3H), 4.32-4.43 (m, 2H), 6.70 (dd, J=3.3, 0.8 Hz, 1H), 6.89-6.93 (m, 1H), 7.10 (d, J=3.2 Hz, 1H), 7.20 (dd, J=8.7, 1.9 Hz, 1H), 7.67 (t, J=1.7 Hz, 1H).

### Example 225

### N-[(butylamino)carbonyl]-2-[5-(5-chloro-6-methoxy-1H-indol-1-yl)-1,3-dimethyl-1H-pyrazol-4-yl]ethanesulfonamide

By a method similar to that in Example 208, the title compound was obtained from 2-[5-(5-chloro-6-methoxy-1H-indol-1-yl)-1,3-dimethyl-1H-pyrazol-4-yl]ethanesulfonamide obtained in Reference Example 200, butylamine and N,N′-carbonyldiimidazole.

¹H-NMR (300 MHz, CDCl₃) δ:0.87-0.95 (m, 3H), 1.22-1.48 (m, 4H), 2.32 (s, 3H), 2.68-2.93 (m, 2H), 3.02-3.16 (m, 4H), 3.49 (s, 3H), 3.83 (s, 3H), 6.10 (br s, 1H), 6.46 (s, 1H), 6.63 (d, J=3.2 Hz, 1H), 6.99 (d, J=3.4 Hz, 1H), 7.67 (s, 1H).

### Example 226

### butyl ({2-[5-(5-chloro-6-methoxy-1H-indol-1-yl)-1,3-dimethyl-1H-pyrazol-4-yl]ethyl}sulfonyl)carbamate

By a method similar to that in Example 186, the title compound was obtained from 2-[5-(5-chloro-6-methoxy-1H-indol-1-yl)-1,3-dimethyl-1H-pyrazol-4-yl]ethanesulfonamide obtained in Reference Example 200, butanol and N,N′-carbonyldiimidazole.

¹H-NMR (300 MHz, CDCl₃) δ:0.89-0.96 (m, 3H), 1.27-1.40 (m, 2H), 1.50-1.61 (m, 2H), 2.33 (s, 3H), 2.69-2.89 (m, 2H), 3.32 (t, J=7.8 Hz, 2H), 3.48 (s, 3H), 3.85 (s, 3H), 4.04 (t, J=6.6 Hz, 2H), 6.47 (s, 1H), 6.63 (dd, J=3.4, 0.8 Hz, 1H), 6.99 (d, J=3.2 Hz, 1H), 7.67 (s, 1H).

### Example 227

### (2E)-N-(butylsulfonyl)-3-{1,3-dimethyl-5-[5-(trifluoromethyl)-1H-pyrrolo[2,3-b]pyridin-1-yl]-1H-pyrazol-4-yl}acrylamide

By a method similar to that in Example 1, the title compound was obtained from (2E)-3-{1,3-dimethyl-5-[5-(trifluoromethyl)-1H-pyrrolo[2,3-b]pyridin-1-yl]-1H-pyrazol-4-yl}acrylic acid obtained in Reference Example 203 and butane-1-sulfonamide.

¹H-NMR (300 MHz, CDCl₃) δ:0.91 (t, J=7.3 Hz, 3H), 1.35-1.49 (m, 2H), 1.67-1.79 (m, 2H), 2.43 (s, 3H), 3.34-3.42 (m, 2H), 3.57 (s, 3H), 5.64 (d, J=15.8 Hz, 1H), 6.90 (d, J=3.8 Hz, 1H), 7.32 (d, J=3.8 Hz, 1H), 7.38 (d, J=15.8 Hz, 1H), 8.32 (d, J=1.5 Hz, 1H), 8.60 (d, J=1.5 Hz, 1H).

### Example 228

### 2-[5-(6-chloro-1H-indol-1-yl)-1,3-dimethyl-1H-pyrazol-4-yl]-N-[(propylamino)carbonyl]ethanesulfonamide

By a method similar to that in Example 208, the title compound was obtained from 2-[5-(6-chloro-1H-indol-1-yl)-1,3-dimethyl-1H-pyrazol-4-yl]ethanesulfonamide obtained in Reference Example 181, propylamine and N,N′-carbonyldiimidazole.

¹H-NMR (300 MHz, CDCl₃) δ:0.88 (t, J=7.3 Hz, 3H), 1.38-1.52 (m, 2H), 2.31 (s, 3H), 2.68-2.91 (m, 2H), 2.96-3.17 (m, 4 H), 3.48 (s, 3H), 6.20 (br s, 1H), 6.73 (dd, J=3.4, 0.8 Hz, 1H), 6.97 (d, J=0.9 Hz, 1H), 7.08 (d, J=3.2 Hz, 1H), 7.19 (dd, J=8.4, 1.8 Hz, 1H), 7.58-7.63 (m, 1H).

### Example 229

### cyclopropylmethyl ({2-[5-(6-chloro-1H-indol-1-yl)-1,3-dimethyl-1H-pyrazol-4-yl]ethyl}sulfonyl)carbamate

By a method similar to that in Example 186, the title compound was obtained from 2-[5-(6-chloro-1H-indol-1-yl)-1,3-dimethyl-1H-pyrazol-4-yl]ethanesulfonamide obtained in Reference Example 181, cyclopropylmethanol and N,N′-carbonyldiimidazole.

¹H-NMR (300 MHz, CDCl₃) δ:0.23-0.30 (m, 2H), 0.53-0.61 (m, 2 H), 0.99-1.12 (m, 1H), 2.32 (s, 3H), 2.65-2.91 (m, 2H), 3.34 (t, J=7.9 Hz, 2H), 3.46 (s, 3H), 3.89 (d, J=7.3 Hz, 2H), 6.73 (d, J=3.4 Hz, 1H), 6.98 (s, 1H), 7.09 (d, J=3.2 Hz, 1H), 7.18 (dd, J=8.5, 1.7 Hz, 1H), 7.60 (d, J=8.3 Hz, 1H).

### Example 230

### 2-[5-(5-chloro-1H-indol-1-yl)-1,3-dimethyl-1H-pyrazol-4-yl]-N-{[(2,2-dimethylpropyl)amino]carbonyl}ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, 2,2-dimethylpropan-1-amine and N,N′-carbonyldiimidazole.

¹H-NMR (300 MHz, CDCl₃) δ:0.85 (s, 9H), 2.30 (s, 3H), 2.69-2.86 (m, 2H), 2.91-2.95 (m, 2H), 2.98-3.12 (m, 2H), 3.47 (s, 3H), 6.29 (br s, 1H), 6.70 (dd, J=3.3, 0.8 Hz, 1H), 6.90 (d, J=8.7 Hz, 1H), 7.10 (d, J=3.4 Hz, 1H), 7.19 (dd, J=8.7, 1.9 Hz, 1H), 7.67 (d, J=1.7 Hz, 1H).

### Example 231

### (2E)-3-[5-(5-chloro-1H-pyrrolo[2,3-b]pyridin-1-yl)-1,3-dimethyl-1H-pyrazol-4-yl]-N-(piperidin-1-ylsulfonyl)acrylamide

By a method similar to that in Example 1, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and piperidine-1-sulfonamide obtained in Reference Example 177.

¹H-NMR (300 MHz, CDCl₃) δ:1.21-1.27 (m, 1H), 1.47-1.54 (m, 2H), 1.57-1.65 (m, 3H), 2.43 (s, 3H), 3.23-3.30 (m, 4H), 3.58 (s, 3H), 5.60 (d, J=15.8 Hz, 1H), 6.75 (d, J=3.8 Hz, 1H), 7.22 (d, J=3.6 Hz, 1H), 7.37 (d, J=15.8 Hz, 1H), 8.02 (d, J=2.3 Hz, 1H), 8.28 (d, J=2.1 Hz, 1H).

### Example 232

### (2E)-3-[1,3-dimethyl-5-(5-methyl-1H-pyrrolo[2,3-b]pyridin-1-yl)-1H-pyrazol-4-yl]-N-(pentylsulfonyl)acrylamide

A mixture of (2E)-3-[1,3-dimethyl-5-(5-methyl-1H-pyrrolo[2,3-b]pyridin-1-yl)-1H-pyrazol-4-yl]acrylic acid obtained in Reference Example 206 (346 mg), 2-methyl-6-nitrobenzoic anhydride (483 mg), pentane-1-sulfonamide (185 mg), triethylamine (354 mg), 4-dimethylaminopyridine (142 mg) and acetonitrile (12 mL) was stirred at room temperature for 18 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and crystallized from diisopropyl ether-ethanol to give the title compound (448 mg, yield 89%) as colorless crystals. melting point 211.1-212.9° C.

¹H-NMR (300 MHz, CDCl₃) δ:0.88 (t, J=7.0 Hz, 3H), 1.25-1.44 (m, 4H), 1.73-1.87 (m, 2H), 2.30 (s, 3H), 2.48 (s, 3H), 3.38-3.47 (m, 2H), 3.52 (s, 3H), 5.53 (d, J=15.6 Hz, 1H), 6.67 (d, J=3.6 Hz, 1H), 7.11 (d, J=3.6 Hz, 1H), 7.35 (d, J=15.8 Hz, 1H), 7.81 (s, 1H), 8.13 (d, J=1.5 Hz, 1H).

### Example 233

### 3-methylbutyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, 3-methylbutan-1-ol and N,N′-carbonyldiimidazole.

¹H-NMR (300 MHz, CDCl₃) δ:0.91 (d, J=6.6 Hz, 6H), 1.43-1.51 (m, 2H), 1.58-1.70 (m, 1H), 2.31 (s, 3H), 2.65-2.87 (m, 2 H), 3.30 (t, J=7.9 Hz, 2H), 3.46 (s, 3H), 4.08 (t, J=6.9 Hz, 2H), 6.70 (d, J=3.2 Hz, 1H), 6.92 (d, J=8.7 Hz, 1H), 7.12 (d, J=3.2 Hz, 1H), 7.20 (dd, J=8.9, 1.9 Hz, 1H), 7.67 (d, J=1.7 Hz, 1H).

### Example 234

### 2-[5-(6-chloro-1H-indol-1-yl)-1,3-dimethyl-1H-pyrazol-4-yl]-N-{[(cyclopropylmethyl)amino]carbonyl}ethanesulfonamide

By a method similar to that in Example 208, the title compound was obtained from 2-[5-(6-chloro-1H-indol-1-yl)-1,3-dimethyl-1H-pyrazol-4-yl]ethanesulfonamide obtained in Reference Example 181, cyclopropylmethylamine and N,N′-carbonyldiimidazole.

¹H-NMR (300 MHz, CDCl₃) δ:0.12-0.18 (m, 2H), 0.42-0.51 (m, 2H), 0.79-0.91 (m, 1H), 2.30 (s, 3H), 2.67-2.90 (m, 2H), 2.98 (dd, J=7.1, 5.6 Hz, 2H), 3.03-3.19 (m, 2H), 3.47 (s, 3H), 6.27 (br s, 1H), 6.73 (dd, J=3.3, 0.8 Hz, 1H), 6.97 (d, J=0.9 Hz, 1H), 7.08 (d, J=3.4 Hz, 1H), 7.18 (dd, J=8.5, 1.7 Hz, 1H), 7.60 (d, J=8.5 Hz, 1H).

### Example 235

### cyclopropylmethyl ({2-[5-(5-chloro-1H-pyrrolo[2,3-b]pyridin-1-yl)-1,3-dimethyl-1H-pyrazol-4-yl]ethyl}sulfonyl)carbamate

By a method similar to that in Example 186, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184, cyclopropylmethanol and N,N′-carbonyldiimidazole.

¹H-NMR (300 MHz, CDCl₃) δ:0.30 (t, J=5.6 Hz, 2H), 0.53-0.61 (m, 2H), 1.02-1.15 (m, 1H), 2.30 (s, 3H), 2.78-2.87 (m, 2H), 3.13 (br s, 1H), 3.36 (s, 3H), 3.71-3.82 (m, 1H), 3.90-4.05 (m, 2H), 6.70 (d, J=3.8 Hz, 1H), 7.16 (d, J=3.6 Hz, 1H), 8.04 (d, J=2.3 Hz, 1H), 8.31 (d, J=2.3 Hz, 1H), 10.60 (br s, 1H).

### Example 236

### 4,4,4-trifluorobutyl ({2-[5-(5-chloro-1H-indol-1-yl)-1,3-dimethyl-1H-pyrazol-4-yl]ethyl}sulfonyl)carbamate

By a method similar to that in Example 186, the title compound was obtained from 2-[5-(5-chloro-1H-indol-1-yl)-1,3-dimethyl-1H-pyrazol-4-yl]ethanesulfonamide obtained in Reference Example 179, 4,4,4-trifluorobutan-1-ol and N,N′-carbonyldiimidazole.

¹H-NMR (300 MHz, CDCl₃) δ:1.79-1.92 (m, 2H), 2.04-2.20 (m, 2H), 2.31 (s, 3H), 2.66-2.88 (m, 2H), 3.28 (t, J=7.8 Hz, 2H), 3.47 (s, 3H), 4.08 (t, J=6.4 Hz, 2H), 6.70 (dd, J=3.2, 0.8 Hz, 1H), 6.92 (d, J=8.7 Hz, 1H), 7.12 (d, J=3.4 Hz, 1H), 7.21 (dd, J=8.7, 2.1 Hz, 1H), 7.67 (d, J=1.7 Hz, 1H).

### Example 237

### isobutyl ({2-[5-(5-chloro-1H-pyrrolo[2,3-b]pyridin-1-yl)-1,3-dimethyl-1H-pyrazol-4-yl]ethyl}sulfonyl)carbamate

By a method similar to that in Example 186, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184, isobutanol and N,N′-carbonyldiimidazole.

¹H-NMR (300 MHz, CDCl₃) δ:0.92 (d, J=6.8 Hz, 6H), 1.82-1.97 (m, 1H), 2.31 (s, 3H), 2.79-2.88 (m, 2H), 3.10-3.22 (m, 1H), 3.34 (s, 3H), 3.66-3.77 (m, 1H), 3.85-4.10 (m, 2H), 6.70 (d, J=3.8 Hz, 1H), 7.16 (d, J=3.6 Hz, 1H), 8.04 (d, J=2.3 Hz, 1H), 8.27 (d, J=2.3 Hz, 1H), 10.52 (br s, 1H).

### Example 238

### N-({2-[5-(5-chloro-1H-indol-1-yl)-1,3-dimethyl-1H-pyrazol-4-yl]ethyl}sulfonyl)piperidine-1-carboxamide

To a solution of butyl ({2-[5-(5-chloro-1H-indol-1-yl)-1,3-dimethyl-1H-pyrazol-4-yl]ethyl}sulfonyl)carbamate obtained in Example 186 (347 mg) in toluene (8 mL) was added piperidine (196 mg), and the mixture was stirred at 90° C. for 4 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 30:70, v/v) to give the title compound (118 mg, yield 33%) as a colorless amorphous solid.

¹H-NMR (300 MHz, CDCl₃) δ:1.45-1.65 (m, 6H), 2.31 (s, 3H), 2.61-2.86 (m, 2H), 3.19-3.26 (m, 4H), 3.41-3.53 (m, 5H), 6.68 (dd, J=3.3, 0.8 Hz, 1H), 6.92 (d, J=8.7 Hz, 1H), 7.15-7.21 (m, 2H), 7.65 (d, J=1.7 Hz, 1H).

### Example 239

### propyl ({2-[5-(5-chloro-1H-pyrrolo[2,3-b]pyridin-1-yl)-1,3-dimethyl-1H-pyrazol-4-yl]ethyl}sulfonyl)carbamate

By a method similar to that in Example 186, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184, propanol and N,N′-carbonyldiimidazole.

¹H-NMR (300 MHz, CDCl₃) δ:0.93 (t, J=7.4 Hz, 3H), 1.59-1.70 (m, 2H), 2.31 (s, 3H), 2.78-2.87 (m, 2H), 3.10-3.21 (m, 1H), 3.35 (s, 3H), 3.86-4.11 (m, 3H), 6.70 (d, J=3.6 Hz, 1H), 7.16 (d, J=3.6 Hz, 1H), 8.04 (d, J=2.3 Hz, 1H), 8.28 (d, J=2.3 Hz, 1H), 10.60 (br s, 1H).

### Example 240

### 4,4,4-trifluorobutyl ({2-[5-(5-chloro-1H-pyrrolo[2,3-b]pyridin-1-yl)-1,3-dimethyl-1H-pyrazol-4-yl]ethyl}sulfonyl)carbamate

By a method similar to that in Example 186, the title compound was obtained from 2-[5-(5-chloro-1H-pyrrolo[2,3-b]pyridin-1-yl)-1,3-dimethyl-1H-pyrazol-4-yl]ethanesulfonamide obtained in Reference Example 184, 4,4,4-trifluorobutan-1-ol and N,N′-carbonyldiimidazole.

¹H-NMR (300 MHz, CDCl₃) δ:1.84-1.96 (m, 2H), 2.08-2.24 (m, 2H), 2.30 (s, 3H), 2.79-2.88 (m, 2H), 3.18 (br s, 1H), 3.34 (s, 3H), 3.95-4.21 (m, 3H), 6.71 (d, J=3.6 Hz, 1H), 7.16 (d, J=3.6 Hz, 1H), 8.05 (d, J=2.3 Hz, 1H), 8.27 (d, J=2.1 Hz, 1H), 10.88 (br s, 1H).

### Example 241

### 2-[5-(5-chloro-1H-indol-1-yl)-1,3-dimethyl-1H-pyrazol-4-yl]-N-{[(4,4,4-trifluorobutyl)amino]carbonyl}ethanesulfonamide

By a method similar to that in Example 208, the title compound was obtained from 2-[5-(5-chloro-1H-indol-1-yl)-1,3-dimethyl-1H-pyrazol-4-yl]ethanesulfonamide obtained in Reference Example 179, 4,4,4-trifluorobutan-1-amine and N,N′-carbonyldiimidazole.

¹H-NMR (300 MHz, CDCl₃) δ:1.66-1.79 (m, 2H), 2.00-2.17 (m, 2H), 2.30 (s, 3H), 2.69-2.88 (m, 2H), 2.94-3.09 (m, 2H), 3.17 (q, J=6.8 Hz, 2H), 3.48 (s, 3H), 6.21 (br s, 1H), 6.71 (d, J=2.7 Hz, 1H), 6.89-6.94 (m, 1H), 7.11 (d, J=3.4 Hz, 1H), 7.20 (dd, J=8.7, 1.9 Hz, 1H), 7.67 (d, J=1.9 Hz, 1H).

### Example 242

### (2E)-N-[(butylamino)sulfonyl]-3-[1,3-dimethyl-5-(5-methyl-1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(5-methyl-1H-pyrrolo[2,3-b]pyridin-1-yl)-1H-pyrazol-4-yl]acrylic acid obtained in Reference Example 206 and N-butylsulfamide obtained in Reference Example 111.

¹H-NMR (300 MHz, CDCl₃) δ:0.89 (t, J=7.3 Hz, 3H), 1.27-1.41 (m, 2H), 1.45-1.56 (m, 2H), 2.30 (s, 3H), 2.48 (s, 3H), 3.03 (q, J=6.7 Hz, 2H), 3.51 (s, 3H), 5.22-5.29 (m, 1H), 5.45 (d, J=15.8 Hz, 1H), 6.67 (d, J=3.6 Hz, 1H), 7.10 (d, J=3.8 Hz, 1H), 7.33 (d, J=15.8 Hz, 1H), 7.81 (d, J=1.1 Hz, 1H), 8.12 (d, J=1.3 Hz, 1H).

### Example 243

### (2E)-3-[5-(2,3-dihydro-1H-pyrrolo[2,3-b]pyridin-1-yl)-1,3-dimethyl-1H-pyrazol-4-yl]-N-(pentylsulfonyl)acrylamide

By a method similar to that in Example 1, the title compound was obtained from (2E)-3-[5-(2,3-dihydro-1H-pyrrolo[2,3-b]pyridin-1-yl)-1,3-dimethyl-1H-pyrazol-4-yl]acrylic acid obtained in Reference Example 215 and pentane-1-sulfonamide.

¹H-NMR (300 MHz, CDCl₃) δ:0.90 (t, J=7.2 Hz, 3H), 1.30-1.48 (m, 4H), 1.78-1.92 (m, 2H), 2.13 (s, 3H), 3.16-3.26 (m, 2H), 3.45-3.54 (m, 2H), 3.63 (s, 3H), 3.76-3.94 (m, 2H), 5.84 (d, J=15.9 Hz, 1H), 6.71 (dd, J=7.2, 4.8 Hz, 1H), 7.35-7.42 (m, 2H), 7.88 (d, J=4.8 Hz, 1H), 10.63 (s, 1H).

### Example 244

### (2E)-3-[5-(2,3-dihydro-1H-pyrrolo[2,3-b]pyridin-1-yl)-1,3-dimethyl-1H-pyrazol-4-yl]-N-[(4-methylphenyl)sulfonyl]acrylamide

By a method similar to that in Example 1, the title compound was obtained from (2E)-3-[5-(2,3-dihydro-1H-pyrrolo[2,3-b]pyridin-1-yl)-1,3-dimethyl-1H-pyrazol-4-yl]acrylic acid obtained in Reference Example 215 and 4-methylbenzenesulfonamide.

¹H-NMR (300 MHz, DMSO-d₆) δ:2.24 (s, 3H), 2.37 (s, 3H), 3.20-3.31 (m, 2H), 3.52 (s, 3H), 3.78-3.86 (m, 2H), 6.17 (d, J=15.8 Hz, 1H), 6.67 (dd, J=7.1, 5.2 Hz, 1H), 7.18 (d, J=15.8 Hz, 1H), 7.40 (d, J=8.1 Hz, 2H), 7.50 (dd, J=7.1, 1.2 Hz, 1H), 7.71-7.75 (m, 1H), 7.79 (d, J=8.1 Hz, 2H), 12.01 (s, 1H).

### Example 245

### (2E)-N-[(butylamino)sulfonyl]-3-[5-(2,3-dihydro-1H-pyrrolo[2,3-b]pyridin-1-yl)-1,3-dimethyl-1H-pyrazol-4-yl]acrylamide

By a method similar to that in Example 62, the title compound was obtained from (2E)-3-[5-(2,3-dihydro-1H-pyrrolo[2,3-b]pyridin-1-yl)-1,3-dimethyl-1H-pyrazol-4-yl]acrylic acid obtained in Reference Example 215 and N-butylsulfamide obtained in Reference Example 111.

¹H-NMR (300 MHz, DMSO-d₆) δ:0.81 (t, J=7.3 Hz, 3H), 1.17-1.45 (m, 4H), 2.28 (s, 3H), 2.78-2.87 (m, 2H), 3.22-3.38 (m, 2H), 3.54 (s, 3H), 3.81-3.89 (m, 2H), 6.21 (d, J=16.0 Hz, 1 H), 6.68 (dd, J=7.2, 5.3 Hz, 1H), 7.26 (d, J=16.0 Hz, 1H), 7.47-7.58 (m, 2H), 7.74-7.78 (m, 1H), 11.31 (s, 1H).

### Example 246

### (2E)-N-[(butylamino)sulfonyl]-3-[3-cyclopropyl-1-methyl-5-(1H-pyrrolo[2,3-b]pyridin-1-yl)-1H-pyrazol-4-yl]acrylamide

By a method similar to that in Example 62, the title compound was obtained from (2E)-3-[3-cyclopropyl-1-methyl-5-(1H-pyrrolo[2,3-b]pyridin-1-yl)-1H-pyrazol-4-yl]acrylic acid obtained in Reference Example 218 and N-butylsulfamide obtained in Reference Example 111.

¹H-NMR (300 MHz, CDCl₃) δ:0.57-0.70 (m, 1H), 0.75-0.98 (m, 6H), 1.26-1.40 (m, 2H), 1.42-1.56 (m, 2H), 1.65-1.80 (m, 1H), 2.96-3.03 (m, 2H), 3.55 (s, 3H), 5.11-5.18 (m, 1H), 5.75 (d, J=15.7 Hz, 1H), 6.78 (d, J=3.4 Hz, 1H), 7.19 (d, J=3.4 Hz, 1H), 7.21-7.26 (m, 1H), 7.38 (d, J=15.7 Hz, 1H), 8.06 (dd, J=8.0, 1.5 Hz, 1H), 8.27-8.35 (m, 1H), 9.20 (br s, 1H).

### Example 247

### (2E)-N-[(butylamino)sulfonyl]-3-[3-cyclopropyl-5-(2,3-dihydro-1H-pyrrolo[2,3-b]pyridin-1-yl)-1-methyl-1H-pyrazol-4-yl]acrylamide

By a method similar to that in Example 62, the title compound was obtained from (2E)-3-[3-cyclopropyl-5-(2,3-dihydro-1H-pyrrolo[2,3-b]pyridin-1-yl)-1-methyl-1H-pyrazol-4-yl]acrylic acid obtained in Reference Example 221 and N-butylsulfamide obtained in Reference Example 111.

¹H-NMR (300 MHz, CDCl₃) δ:0.45-0.58 (m, 1H), 0.60-0.80 (m, 1H), 0.82-0.88 (m, 2H), 0.92 (t, J=7.2 Hz, 3H), 1.32-1.46 (m, 2H), 1.50-1.69 (m, 3H), 3.08-3.18 (m, 2H), 3.20-3.30 (m, 2H), 3.64 (s, 3H), 3.82-3.95 (m, 2H), 5.25-5.32 (m, 1H), 6.04 (d, J=15.7 Hz, 1H), 6.67 (dd, J=7.3, 5.0 Hz, 1H), 7.39 (d, J=7.3 Hz, 1H), 7.44 (d, J=15.7 Hz, 1H), 7.83 (d, J=5.0 Hz, 1H), 10.61 (br s, 1H).

### Example 248

### (2E)-N-{[(cyclopropylmethyl)amino]sulfonyl}-3-[1,3-dimethyl-5-(1H-pyrrolo[2,3-b]pyridin-1-yl)-1H-pyrazol-4-yl]acrylamide

By a method similar to that in Example 62, the title compound was obtained from (2E)-3-[1,3-dimethyl-5-(1H-pyrrolo[2,3-b]pyridin-1-yl)-1H-pyrazol-4-yl]acrylic acid obtained in Reference Example 13 and N-(cyclopropylmethyl)sulfamide obtained in Reference Example 115.

¹H-NMR (300 MHz, CDCl₃) δ:0.14-0.20 (m, 2H), 0.49-0.55 (m, 2H), 0.88-1.02 (m, 1H), 2.30 (s, 3H), 2.86-2.91 (m, 2H), 3.56 (s, 3H), 5.32 (t, J=5.9 Hz, 1H), 5.47 (d, J=15.5 Hz, 1H), 6.79 (d, J=3.6 Hz, 1H), 7.18 (d, J=3.6 Hz, 1H), 7.23 (dd, J=8.0, 4.9 Hz, 1H), 7.33 (d, J=15.5 Hz, 1H), 8.05 (dd, J=8.0, 1.5 Hz, 1H), 8.27-8.34 (m, 1H), 8.77 (s, 1H).

### Example 249

### (2E)-N-{[(cyclopropylmethyl)amino]sulfonyl}-3-[5-(6-methoxy-1H-indol-1-yl)-1,3-dimethyl-1H-pyrazol-4-yl]acrylamide

By a method similar to that in Example 62, the title compound was obtained from (2E)-3-[5-(6-methoxy-1H-indol-1-yl)-1,3-dimethyl-1H-pyrazol-4-yl]acrylic acid obtained in Reference Example 60 and N-(cyclopropylmethyl)sulfamide obtained in Reference Example 115.

¹H-NMR (300 MHz, CDCl₃) δ:0.10-0.17 (m, 2H), 0.44-0.53 (m, 2H), 0.83-0.92 (m, 1H), 2.44 (s, 3H), 2.80 (d, J=6.5 Hz, 2H), 3.55 (s, 3H), 3.76 (s, 3H), 5.14-5.20 (m, 2H), 6.40 (d, J=2.2 Hz, 1H), 6.75 (dd, J=3.4, 0.8 Hz, 1H), 6.90 (dd, J=8.7, 2.2 Hz, 1H), 6.94 (d, J=3.4 Hz, 1H), 7.47 (d, J=15.8 Hz, 1H), 7.58-7.63 (m, 2H).

### Example 250

### (2E)-3-[1-methyl-5-(1H-pyrrolo[2,3-b]pyridin-1-yl)-3-(trifluoromethyl)-1H-pyrazol-4-yl]-N-(pentylsulfonyl)acrylamide

By a method similar to that in Example 1, the title compound was obtained from (2E)-3-[1-methyl-5-(1H-pyrrolo[2,3-b]pyridin-1-yl)-3-(trifluoromethyl)-1H-pyrazol-4-yl]acrylic acid obtained in Example 230 and pentane-1-sulfonamide.

¹H-NMR (300 MHz, CDCl₃) δ:0.88 (t, J=7.0 Hz, 3H), 1.25-1.42 (m, 4H), 1.71-1.84 (m, 2H), 3.34-3.40 (m, 2H), 3.70 (s, 3H), 5.61 (d, J=15.9 Hz, 1H), 6.84 (d, J=3.8 Hz, 1H), 7.18 (d, J=3.8 Hz, 1H), 7.26-7.31 (m, 1H), 7.41 (d, J=15.9 Hz, 1H), 8.06-8.10 (m, 1H), 8.33-8.35 (m, 1H), 8.40 (s, 1H).

### Example 251

### (2E)-3-[5-(3-chloro-1H-pyrrolo[2,3-b]pyridin-1-yl)-1,3-dimethyl-1H-pyrazol-4-yl]-N-[(4-methylphenyl)sulfonyl]acrylamide

By a method similar to that in Example 1, the title compound was obtained from (2E)-3-[5-(3-chloro-1H-pyrrolo[2,3-b]pyridin-1-yl)-1,3-dimethyl-1H-pyrazol-4-yl]acrylic acid obtained in Reference Example 133 and 4-methylbenzenesulfonamide.

¹H-NMR (300 MHz, CDCl₃) δ:2.36 (s, 3H), 2.40 (s, 3H), 3.55 (s, 3H), 5.67 (d, J=15.8 Hz, 1H), 7.15 (s, 1H), 7.23-7.34 (m, 4H), 7.85 (d, J=8.5 Hz, 2H), 8.06 (dd, J=7.9, 1.5 Hz, 1H), 8.20 (br s, 1H), 8.38 (dd, J=4.7, 1.5 Hz, 1H).

### Example 252

### (2E)-3-[5-(3-chloro-1H-pyrrolo[2,3-b]pyridin-1-yl)-1,3-dimethyl-1H-pyrazol-4-yl]-N-(pentylsulfonyl)acrylamide

By a method similar to that in Example 1, the title compound was obtained from (2E)-3-[5-(3-chloro-1H-pyrrolo[2,3-b]pyridin-1-yl)-1,3-dimethyl-1H-pyrazol-4-yl]acrylic acid obtained in Reference Example 133 and pentane-1-sulfonamide.

¹H-NMR (300 MHz, CDCl₃) δ:0.88 (t, J=7.1 Hz, 3H), 1.25-1.46 (m, 4H), 1.72-1.86 (m, 2H), 2.36 (s, 3H), 3.35-3.46 (m, 2H), 3.54 (s, 3H), 5.66 (d, J=15.8 Hz, 1H), 7.19 (s, 1H), 7.29-7.40 (m, 2H), 8.08 (dd, J=7.9, 1.5 Hz, 1H), 8.39 (dd, J=4.8, 1.5 Hz, 1H), 8.49 (s, 1H).

### Example 253

### (2E)-3-[1,3-dimethyl-5-(1H-pyrrolo[3,2-c]pyridin-1-yl)-1H-pyrazol-4-yl]-N-(pentylsulfonyl)acrylamide

By a method similar to that in Example 1, the title compound was obtained from (2E)-3-[1,3-dimethyl-5-(1H-pyrrolo[3,2-c]pyridin-1-yl)-1H-pyrazol-4-yl]acrylic acid obtained in Reference Example 224 and pentane-1-sulfonamide.

¹H-NMR (300 MHz, CDCl₃) δ:0.83 (t, J=7.2 Hz, 3H), 1.24-1.35 (m, 4H), 1.65-1.75 (m, 2H), 2.46 (s, 3H), 3.34-3.41 (m, 2H), 3.44 (s, 3H), 5.54 (d, J=16.1 Hz, 1H), 6.85 (d, J=3.4 Hz, 1H), 6.95 (d, J=6.1 Hz, 1H), 7.16 (d, J=3.4 Hz, 1H), 7.68 (d, J=16.1 Hz, 1H), 8.06 (d, J=6.1 Hz, 1H), 8.24 (s, 1H).

### Example 254

### (2E)-3-[1,3-dimethyl-5-(1H-pyrrolo[2,3-c]pyridin-1-yl)-1H-pyrazol-4-yl]-N-(pentylsulfonyl)acrylamide

By a method similar to that in Example 1, the title compound was obtained from (2E)-3-[1,3-dimethyl-5-(1H-pyrrolo[2,3-c]pyridin-1-yl)-1H-pyrazol-4-yl]acrylic acid obtained in Reference Example 227 and pentane-1-sulfonamide.

¹H-NMR (300 MHz, CDCl₃) δ:0.86 (t, J=7.2 Hz, 3H), 1.26-1.42 (m, 4H), 1.66-1.78 (m, 2H), 2.43 (s, 3H), 3.32-3.40 (m, 2H), 3.45 (s, 3H), 5.51 (d, J=15.9 Hz, 1H), 6.87 (d, J=3.2 Hz, 1H), 7.25 (d, J=3.2 Hz, 1H), 7.49 (d, J=15.9 Hz, 1H), 7.55 (d, J=4.9 Hz, 1H), 8.11 (d, J=4.9 Hz, 1H), 8.21 (s, 1H).

### Example 255

### potassium {(2E)-3-[1,3-dimethyl-5-(1H-pyrrolo[2,3-c]pyridin-1-yl)-1H-pyrazol-4-yl]prop-2-enoyl}(pentylsulfonyl)azanide

By a method similar to that in Example 7, the title compound was obtained from (2E)-3-[1,3-dimethyl-5-(1H-pyrrolo[2,3-c]pyridin-1-yl)-1H-pyrazol-4-yl]-N-(pentylsulfonyl)acrylamide obtained in Example 254.

¹H-NMR (300 MHz, DMSO-d₆) δ:0.77-0.83 (m, 3H), 1.18-1.23 (m, 4H), 1.37-1.46 (m, 2H), 2.35 (s, 3H), 2.81-2.88 (m, 2H), 3.46 (s, 3H), 5.52 (d, J=16.2 Hz, 1H), 6.76 (d, J=16.2 Hz, 1H), 6.91 (d, J=2.9 Hz, 1H), 7.71 (dd, J=5.4, 0.9 Hz, 1H), 7.80 (d, J=2.9 Hz, 1H), 8.28 (d, J=5.4 Hz, 1H), 8.37 (s, 1H).

### Example 256

### (2E)-N-[(butylamino)sulfonyl]-3-[1,3-dimethyl-5-(1H-pyrrolo[2,3-c]pyridin-1-yl)-1H-pyrazol-4-yl]acrylamide

By a method similar to that in Example 62, the title compound was obtained from (2E)-3-[1,3-dimethyl-5-(1H-pyrrolo[2,3-c]pyridin-1-yl)-1H-pyrazol-4-yl]acrylic acid obtained in Reference Example 227 and N-butylsulfamide obtained in Reference Example 111.

¹H-NMR (300 MHz, DMSO-d₆) δ:0.79 (t, J=7.4 Hz, 3H), 1.15-1.40 (m, 4H), 2.40 (s, 3H), 2.74-2.81 (m, 2H), 3.51 (s, 3H), 6.05 (d, J=16.1 Hz, 1H), 6.94 (d, J=3.2 Hz, 1H), 7.00 (d, J=16.1 Hz, 1H), 7.53 (t, J=5.7 Hz, 1H), 7.74 (d, J=5.5 Hz, 1H), 7.85 (d, J=3.2 Hz, 1H), 8.31 (d, J=5.5 Hz, 1H), 8.41 (s, 1H), 11.31 (s, 1H).

### Example 257

### sodium {(2E)-3-[5-(6-methoxy-1H-indol-1-yl)-1,3-dimethyl-1H-pyrazol-4-yl]prop-2-enoyl}(pentylsulfonyl)azanide

By a method similar to that in Example 28, the title compound was obtained from (2E)-3-[5-(6-methoxy-1H-indol-1-yl)-1,3-dimethyl-1H-pyrazol-4-yl]-N-(pentylsulfonyl)acrylamide obtained in Example 38.

¹H-NMR (300 MHz, DMSO-d₆) δ:0.77-0.84 (m, 3H), 1.18-1.24 (m, 4H), 1.42-1.48 (m, 2H), 2.34 (s, 3H), 2.82-2.88 (m, 2H), 3.42 (s, 3H), 3.68 (s, 3H), 5.61 (d, J=15.9 Hz, 1H), 6.43 (d, J=1.9 Hz, 1H), 6.71 (d, J=3.4 Hz, 1H), 6.75-6.84 (m, 2H), 7.33 (d, J=3.4 Hz, 1H), 7.57 (d, J=8.7 Hz, 1H).

### Example 258

### (2E)-3-[5-(3-chloro-1H-pyrrolo[2,3-b]pyridin-1-yl)-1,3-dimethyl-1H-pyrazol-4-yl]-N-[(1-methyl-1H-imidazol-4-yl)sulfonyl]acrylamide

By a method similar to that in Example 1, the title compound was obtained from (2E)-3-[5-(3-chloro-1H-pyrrolo[2,3-b]pyridin-1-yl)-1,3-dimethyl-1H-pyrazol-4-yl]acrylic acid obtained in Reference Example 133 and 1-methyl-1H-imidazole-4-sulfonamide.

¹H-NMR (300 MHz, DMSO-d₆) δ:2.36 (s, 3H), 3.48 (s, 3H), 3.67 (s, 3H), 6.06 (d, J=16.0 Hz, 1H), 6.93 (d, J=16.0 Hz, 1H), 7.40 (dd, J=7.9, 4.7 Hz, 1H), 7.73 (d, J=1.1 Hz, 1H), 7.87 (d, J=1.1 Hz, 1H), 8.04 (s, 1H), 8.16 (dd, J=7.9, 1.5 Hz, 1H), 8.36 (dd, J=4.7, 1.5 Hz, 1H), 11.87 (s, 1H).

### Example 259

### (2E)-3-[5-(5-fluoro-1H-indol-1-yl)-1,3-dimethyl-1H-pyrazol-4-yl]-N-[(2-methoxy-4-methylphenyl)sulfonyl]acrylamide

By a method similar to that in Example 1, the title compound was obtained from (2E)-3-[5-(5-fluoro-1H-indol-1-yl)-1,3-dimethyl-1H-pyrazol-4-yl]acrylic acid obtained in Reference Example 21 and 2-methoxy-4-methylbenzenesulfonamide.

¹H-NMR (300 MHz, CDCl₃) δ:2.35 (s, 3H), 2.39 (s, 3H), 3.48 (s, 3H), 3.77 (s, 3H), 5.59 (d, J=15.8 Hz, 1H), 6.70 (d, J=3.4 Hz, 1H), 6.74 (s, 1H), 6.79-6.87 (m, 2H), 6.90-7.00 (m, 1H), 7.07 (d, J=3.4 Hz, 1H), 7.22-7.37 (m, 2H), 7.76 (d, J=8.1 Hz, 1H), 8.26 (s, 1H).

### Example 260

### (2E)-3-[5-(5-fluoro-1H-indol-1-yl)-1,3-dimethyl-1H-pyrazol-4-yl]-N-[(2-hydroxy-4-methylphenyl)sulfonyl]acrylamide

To a solution of (2E)-3-[5-(5-fluoro-1H-indol-1-yl)-1,3-dimethyl-1H-pyrazol-4-yl]-N-[(2-methoxy-4-methylphenyl)sulfonyl]acrylamide obtained in Example 259 (312 mg) in dichloromethane (30 mL) was added dropwise boron tribromide (1M dichloromethane solution, 1.3 mL) with stirring at −78° C., and the mixture was stirred at −78° C. for 1 hr. The mixture was allowed to warm to room temperature, and stirred for 30 hr, and then heated under reflux for 2 hr. The reaction mixture was concentrated under reduced pressure, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 35:65, v/v), and crystallized from hexane-ethanol to give the title compound (220 mg, yield 72%) as colorless crystals.

¹H-NMR (300 MHz, CDCl₃) δ:2.32 (s, 3H), 2.39 (s, 3H), 3.50 (s, 3H), 5.19 (d, J=15.8 Hz, 1H), 6.73-6.88 (m, 4H), 6.94-7.02 (m, 1H), 7.07 (d, J=3.4 Hz, 1H), 7.36 (dd, J=9.0, 2.4 Hz, 1H), 7.43 (d, J=15.8 Hz, 1H), 7.50 (d, J=8.3 Hz, 1H), 7.89 (s, 1H), 8.71 (s, 1H).

### Example 261

### (2E)-3-[3-(1-naphthyl)-2-thienyl]-N-(pentylsulfonyl)acrylamide

By a method similar to that in Example 1, the title compound was obtained from (2E)-3-[3-(1-naphthyl)-2-thienyl]acrylic acid obtained in Reference Example 233 and pentane-1-sulfonamide.

¹H-NMR (300 MHz, CDCl₃) δ:0.83-0.90 (m, 3H), 1.25-1.41 (m, 4H), 1.72-1.82 (m, 2H), 3.35-3.42 (m, 2H), 6.18 (d, J=15.1 Hz, 1H), 7.18 (d, J=4.9 Hz, 1H), 7.33 (d, J=5.7 Hz, 1H), 7.40-7.65 (m, 6H), 7.73 (s, 1H), 7.88-7.98 (m, 2H).

### Example 262

### (2E)-N-[(butylamino)sulfonyl]-3-[3-(1-naphthyl)-2-thienyl]acrylamide

By a method similar to that in Example 62, the title compound was obtained from (2E)-3-[3-(1-naphthyl)-2-thienyl]acrylic acid obtained in Reference Example 233 and N-butylsulfamide obtained in Reference Example 111.

¹H-NMR (300 MHz, CDCl₃) δ:0.86 (t, J=7.3 Hz, 3H), 1.23-1.38 (m, 2H), 1.41-1.54 (m, 2H), 2.91-3.00 (m, 2H), 5.11 (t, J=6.0 Hz, 1H), 6.13 (d, J=15.3 Hz, 1H), 7.18 (d, J=5.1 Hz, 1H), 7.33 (dd, J=7.0, 1.1 Hz, 1H), 7.40-7.67 (m, 6H), 7.84 (s, 1H), 7.89-7.94 (m, 2H).

### Example 263

### (2E)-N-[(butylamino)sulfonyl]-3-[5-(5-chloro-1H-pyrrolo[2,3-b]pyridin-1-yl)-1,3-dimethyl-1H-pyrazol-4-yl]acrylamide

By a method similar to that in Example 62, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and N-butylsulfamide obtained in Reference Example 111.

¹H-NMR (300 MHz, CDCl₃) δ:0.89 (t, J=7.3 Hz, 3H), 1.27-1.40 (m, 2H), 1.44-1.52 (m, 2H), 2.39 (s, 3H), 2.96-3.03 (m, 2H), 3.53 (s, 3H), 5.20 (t, J=6.1 Hz, 1H), 5.44 (d, J=15.6 Hz, 1H), 6.75 (d, J=3.6 Hz, 1H), 7.20 (d, J=3.6 Hz, 1H), 7.35 (d, J=15.6 Hz, 1H), 8.02 (d, J=2.3 Hz, 1H), 8.23 (d, J=2.3 Hz, 1H), 8.42 (s, 1H).

### Example 264

### (2E)-3-[5-(5-chloro-1H-pyrrolo[2,3-b]pyridin-1-yl)-1,3-dimethyl-1H-pyrazol-4-yl]-N-{[(cyclopropylmethyl)amino]sulfonyl}acrylamide

A mixture of (2E)-3-[5-(5-chloro-1H-pyrrolo[2,3-b]pyridin-1-yl)-1,3-dimethyl-1H-pyrazol-4-yl]acrylic acid obtained in Reference Example 235 (380 mg), 2-methyl-6-nitrobenzoic anhydride (494 mg), N-(cyclopropylmethyl)sulfamide obtained in Reference Example 115 (186 mg), triethylamine (372 mg), 4-dimethylaminopyridine (151 mg) and acetonitrile (8 mL) was stirred at room temperature for 18 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 35:65, v/v), and crystallized from hexane-ethanol to give the title compound (290 mg, yield 53%) as colorless crystals. melting point 209.3-210.0° C.

¹H-NMR (300 MHz, CDCl₃) δ:0.13-0.19 (m, 2H), 0.48-0.56 (m, 2H), 0.89-1.03 (m, 1H), 2.40 (s, 3H), 2.86 (dd, J=7.2, 6.0 Hz, 2H), 3.55 (s, 3H), 5.28 (t, J=6.0 Hz, 1H), 5.42 (d, J=15.8 Hz, 1H), 6.75 (d, J=3.6 Hz, 1H), 7.20 (d, J=3.6 Hz, 1H), 7.36 (d, J=15.8 Hz, 1H), 8.03 (d, J=2.3 Hz, 1H), 8.12 (s, 1H), 8.25 (d, J=2.3 Hz, 1H).

### Example 265

### (2E)-3-[5-(5-chloro-1H-pyrrolo[2,3-b]pyridin-1-yl)-1,3-dimethyl-1H-pyrazol-4-yl]-N-(pentylsulfonyl)acrylamide

By a method similar to that in Example 1, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and pentane-1-sulfonamide.

¹H-NMR (300 MHz, CDCl₃) δ:0.88 (t, J=7.1 Hz, 3H), 1.25-1.45 (m, 4H), 1.70-1.82 (m, 2H), 2.40 (s, 3H), 3.36-3.43 (m, 2H), 3.54 (s, 3H), 5.53 (d, J=15.8 Hz, 1H), 6.75 (d, J=3.8 Hz, 1H), 7.20 (d, J=3.8 Hz, 1H), 7.38 (d, J=15.8 Hz, 1H), 8.02 (d, J=2.3 Hz, 1H), 8.19 (s, 1H), 8.25 (d, J=2.3 Hz, 1H).

### Example 266

### (2E)-3-[5-(5-chloro-1H-pyrrolo[2,3-b]pyridin-1-yl)-1,3-dimethyl-1H-pyrazol-4-yl]-N-[(4-methylphenyl)sulfonyl]acrylamide

By a method similar to that in Example 1, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and 4-methylbenzenesulfonamide.

¹H-NMR (300 MHz, CDCl₃) δ:2.36 (s, 3H), 2.41 (s, 3H), 3.54 (s, 3 H), 5.55 (d, J=15.8 Hz, 1H), 6.71 (d, J=3.8 Hz, 1H), 7.17 (d, J=3.8 Hz, 1H), 7.26-7.37 (m, 3H), 7.79-7.86 (m, 2H), 7.94 (d, J=2.2 Hz, 1H), 8.17 (s, 1H), 8.24 (d, J=2.2 Hz, 1H).

### Example 267

### (2E)-3-[5-(5-chloro-1H-pyrrolo[2,3-b]pyridin-1-yl)-1,3-dimethyl-1H-pyrazol-4-yl]-N-[(2-methoxy-4-methylphenyl)sulfonyl]acrylamide

By a method similar to that in Example 1, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and 2-methoxy-4-methylbenzenesulfonamide.

¹H-NMR (300 MHz, CDCl₃) δ:2.35 (s, 3H), 2.39 (s, 3H), 3.53 (s, 3H), 3.83 (s, 3H), 5.83 (d, J=15.8 Hz, 1H), 6.67 (d, J=3.6 Hz, 1H), 6.76 (s, 1H), 6.85 (d, J=8.1 Hz, 1H), 7.17 (d, J=3.6 Hz, 1H), 7.30 (d, J=15.8 Hz, 1H), 7.76 (d, J=8.1 Hz, 1H), 7.92 (d, J=2.3 Hz, 1H), 8.24 (d, J=2.3 Hz, 1H).

### Example 268

### (2E)-3-[5-(5-chloro-1H-pyrrolo[2,3-b]pyridin-1-yl)-1,3-dimethyl-1H-pyrazol-4-yl]-N-[(2-hydroxy-4-methylphenyl)sulfonyl]acrylamide

To a solution of (2E)-3-[5-(5-chloro-1H-pyrrolo[2,3-b]pyridin-1-yl)-1,3-dimethyl-1H-pyrazol-4-yl]-N-[(2-methoxy-4-methylphenyl)sulfonyl]acrylamide obtained in Example 267 (396 mg) in dichloromethane (15 mL) was added dropwise boron tribromide (1M dichloromethane solution, 2.4 mL) with stirring at 0° C., and the mixture was stirred at 0° C. for 4 hr. The reaction mixture was quenched with methanol, and concentrated under reduced pressure. Water was added, and the mixture was extracted with ethyl acetate. The organic layer was washed with saturated brine, dried over anhydrous magnesium sulfate, and filtrated. The filtrate was concentrated, and the residue was crystallized from hexane-ethanol to give the title compound (190 mg, yield 49%) as colorless crystals.

¹H-NMR (300 MHz, DMSO-d₆) δ:2.24 (s, 3H), 2.35 (s, 3H), 3.46 (s, 3H), 6.11 (d, J=16.1 Hz, 1H), 6.69-6.74 (m, 2H), 6.84 (d, J=3.8 Hz, 1H), 6.89 (d, J=16.1 Hz, 1H), 7.55 (d, J=8.3 Hz, 1H), 7.77 (d, J=3.8 Hz, 1H), 8.25-8.30 (m, 2H), 10.64 (s, 1H), 11.80 (s, 1H).

### Example 269

### (2E)-3-[5-(5-bromo-1H-pyrrolo[2,3-b]pyridin-1-yl)-1,3-dimethyl-1H-pyrazol-4-yl]-N-[(butylamino)sulfonyl]acrylamide

By a method similar to that in Example 62, the title compound was obtained from (2E)-3-[5-(5-bromo-1H-pyrrolo[2,3-b]pyridin-1-yl)-1,3-dimethyl-1H-pyrazol-4-yl]acrylic acid obtained in Reference Example 238 and N-butylsulfamide obtained in Reference Example 111.

¹H-NMR (300 MHz, CDCl₃) δ:0.88 (t, J=7.3 Hz, 3H), 1.25-1.40 (m, 2H), 1.44-1.55 (m, 2H), 2.41 (s, 3H), 2.95-3.03 (m, 2H), 3.54 (s, 3H), 5.14 (t, J=6.2 Hz, 1H), 5.43 (d, J=15.8 Hz, 1H), 6.75 (d, J=3.8 Hz, 1H), 7.18 (d, J=3.8 Hz, 1H), 7.37 (d, J=15.8 Hz, 1H), 8.10 (s, 1H), 8.18 (d, J=2.2 Hz, 1H), 8.34 (d, J=2.2 Hz, 1H).

### Example 270

### (2E)-3-[1,3-dimethyl-5-(3-methyl-1H-pyrrolo[2,3-b]pyridin-1-yl)-1H-pyrazol-4-yl]-N-[(2-methoxy-4-methylphenyl)sulfonyl]acrylamide

By a method similar to that in Example 1, the title compound was obtained from (2E)-3-[1,3-dimethyl-5-(3-methyl-1H-pyrrolo[2,3-b]pyridin-1-yl)-1H-pyrazol-4-yl]acrylic acid obtained in Reference Example 136 and 2-methoxy-4-methylbenzenesulfonamide.

¹H-NMR (300 MHz, CDCl₃) δ:2.33 (s, 3H), 2.36 (s, 3H), 2.37 (s, 3H), 3.54 (s, 3H), 3.82 (s, 3H), 5.89 (d, J=15.9 Hz, 1H), 6.73 (s, 1H) 6.78 (d, J=8.3 Hz, 1H), 6.90 (s, 1H), 7.16-7.19 (m, 1H), 7.31 (d, J=15.9 Hz, 1H), 7.68 (d, J=8.0 Hz, 1H), 7.93-7.96 (m, 1H), 8.26-8.32 (m, 1H), 8.40 (s, 1H).

### Example 271

### 3-{2-[5-(5-chloro-1H-indol-1-yl)-1,3-dimethyl-1H-pyrazol-4-yl]ethyl}imidazolidine-2,4-dione

To a solution of imidazolidine-2,4-dione (116 mg) in N,N-dimethylformamide (4 mL), which was cooled at 0° C. in an ice bath, was added 60% sodium hydride (in oil, 49 mg) with stirring, and the mixture was stirred at 0° C. for 20 min. 2-[5-(5-Chloro-1H-indol-1-yl)-1,3-dimethyl-1H-pyrazol-4-yl]ethyl methanesulfonate obtained in Reference Example 239 (329 mg) was added to this reaction mixture at 0° C., and the reaction mixture was stirred at 80° C. for 18 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (ethyl acetate), and crystallized from hexane-ethanol to give the title compound (195 mg, yield 58%) as colorless crystals.

¹H-NMR (300 MHz, DMSO-d₆) δ:2.18 (s, 3H), 2.29-2.46 (m, 2H), 3.26 (t, J=7.0 Hz, 2H), 3.33 (s, 3H), 3.64-3.79 (m, 2H), 6.75 (d, J=3.0 Hz, 1H), 7.06 (d, J=8.7 Hz, 1H), 7.19 (dd, J=8.7, 2.0 Hz, 1H), 7.53 (d, J=3.0 Hz, 1H), 7.74 (d, J=2.0 Hz, 1H), 7.92 (s, 1H).

### Example 272

### 3-{2-[5-(5-chloro-1H-indol-1-yl)-1,3-dimethyl-1H-pyrazol-4-yl]ethyl}-5-propylimidazolidine-2,4-dione

By a method similar to that in Example 271, the title compound was obtained from 2-[5-(5-chloro-1H-indol-1-yl)-1,3-dimethyl-1H-pyrazol-4-yl]ethyl methanesulfonate obtained in Reference Example 239 and 5-propylimidazolidine-2,4-dione.

¹H-NMR (300 MHz, CDCl₃) δ:0.92 (t, J=7.4 Hz, 3H), 1.24-1.43 (m, 2H), 1.46-1.58 (m, 1H), 1.68-1.84 (m, 1H), 2.27 (s, 3 H), 2.38-2.51 (m, 1H), 2.61-2.74 (m, 1H), 3.35-3.47 (m, 5H), 3.70-3.89 (m, 1H), 5.27-5.29 (m, 1H), 6.69 (d, J=3.4 Hz, 1H), 6.99 (d, J=8.7 Hz, 1H), 7.18 (dd, J=8.7, 1.9 Hz, 1H), 7.29 (dd, J=5.7, 3.4 Hz, 1H), 7.66 (d, J=1.9 Hz, 1H).

### Example 273

### 1-{2-[5-(5-chloro-1H-indol-1-yl)-1,3-dimethyl-1H-pyrazol-4-yl]ethyl}piperazin-2-one hydrochloride

To a solution of tert-butyl 4-{2-[5-(5-chloro-1H-indol-1-yl)-1,3-dimethyl-1H-pyrazol-4-yl]ethyl}-3-oxopiperazine-1-carboxylate obtained in Reference Example 240 (223 mg) in ethyl acetate (4 mL) was added a 4M hydrogen chloride-ethyl acetate solution (2 mL), and the mixture was stirred at room temperature for 1 hr. The reaction mixture was concentrated under reduced pressure, and the residue was purified by preparative HPLC (tool and preparative conditions were the same as those in Reference Example 97). The obtained amorphous solid was neutralized with aqueous sodium hydrogencarbonate solution, and the mixture was extracted with ethyl acetate. The organic layer was washed with saturated brine, and dried over anhydrous magnesium sulfate. A 4M hydrogen chloride-ethyl acetate solution (1 mL) was added to the obtained residue, and the mixture was concentrated under reduced pressure, and dried to give the title compound (132 mg, yield 68%) as a colorless amorphous solid.

¹H-NMR (300 MHz, DMSO-d₆) δ:2.24 (s, 3H), 2.25-2.46 (m, 2H), 3.15-3.34 (m, 6H), 3.38 (s, 3H), 3.52-3.60 (m, 2H), 6.77 (d, J=3.3 Hz, 1H), 7.05 (d, J=8.7 Hz, 1H), 7.20 (dd, J=8.7, 1.7 Hz, 1H), 7.64 (d, J=3.3 Hz, 1H), 7.75 (d, J=1.7 Hz, 1H), 9.54 (s, 2H).

### Example 274

### butyl {2-[5-(5-chloro-1H-indol-1-yl)-1,3-dimethyl-1H-pyrazol-4-yl]ethoxy}carbamate

To a solution of 1-{4-[2-(aminooxy)ethyl]-1,3-dimethyl-1H-pyrazol-5-yl}-5-chloro-1H-indole obtained in Reference Example 242 (308 mg) in tetrahydrofuran (8 mL) was added triethylamine (330 mg), and then a solution of butyl chloroformate (172 mg) in tetrahydrofuran (6 mL), and the mixture was stirred at room temperature for 1 hr. The reaction mixture was concentrated under reduced pressure, water was added to the residue, and the mixture was extracted with ethyl acetate. The organic layer was washed with saturated brine, and dried over anhydrous magnesium sulfate. The residue was subjected to silica gel column chromatography (hexane-ethyl acetate 25:75, v/v) to give the title compound (146 mg, yield 35%) as a colorless oil.

¹H-NMR (300 MHz, CDCl₃) δ: 0.91 (t, J=7.4 Hz, 3H), 1.25-1.40 (m, 2H), 1.49-1.62 (m, 2H), 2.29 (s, 3H), 2.45-2.68 (m, 2H), 3.45 (s, 3H), 3.71 (t, J=6.6 Hz, 2H), 4.07 (t, J=6.6 Hz, 2H), 6.68 (d, J=3.4 Hz, 1H), 6.79 (s, 1H), 6.95 (d, J=8.7 Hz, 1H), 7.13 (d, J=3.4 Hz, 1H), 7.18 (dd, J=8.7, 1.7 Hz, 1H), 7.66 (d, J=1.7 Hz, 1H).

### Example 275

### N-{2-[5-(5-chloro-1H-indol-1-yl)-1,3-dimethyl-1H-pyrazol-4-yl]ethoxy}-N′-pentylurea

To a solution of 1-{4-[2-(aminooxy)ethyl]-1,3-dimethyl-1H-pyrazol-5-yl}-5-chloro-1H-indole obtained in Reference Example 242 (300 mg) in tetrahydrofuran (8 mL) was added pentyl isocyanate (175 mg), and the mixture was stirred at room temperature for 4 hr. The reaction mixture was concentrated under reduced pressure, and the residue was subjected to silica gel column chromatography (hexane-ethyl acetate 25:75, v/v), and then purified by preparative HPLC (tool and preparative conditions were the same as those in Reference Example 97) to give the title compound (171 mg, yield 41%) as a colorless oil.

¹H-NMR (300 MHz, CDCl₃) δ: 0.89 (t, J=7.0 Hz, 3H), 1.18-1.47 (m, 6H), 2.30 (s, 3H), 2.45-2.66 (m, 2H), 3.10-3.20 (m, 2H), 3.46 (s, 3H), 3.66 (t, J=6.7 Hz, 2H), 5.29-5.38 (m, 1H), 6.66-6.71 (m, 2H), 6.92 (d, J=8.9 Hz, 1H), 7.10 (d, J=3.2 Hz, 1H), 7.19 (dd, J=8.9, 1.7 Hz, 1H), 7.67 (d, J=1.7 Hz, 1H).

### Example 276

### (4R)-5-{2-[5-(5-chloro-1H-indol-1-yl)-1,3-dimethyl-1H-pyrazol-4-yl]ethyl}-4-isopropyl-1,2,5-thiadiazolidin-3-one 1,1-dioxide

(4R)-5-{2-[5-(5-Chloro-1H-indol-1-yl)-1,3-dimethyl-1H-pyrazol-4-yl]ethyl}-4-isopropyl-2-(4-methoxybenzyl)-1,2,5-thiadiazolidin-3-one 1,1-dioxide obtained in Reference Example 243 (205 mg) was dissolved in trifluoroacetic acid (4 mL), and the solution was stirred with heating at 65° C. for 7 hr. The reaction mixture was concentrated under reduced pressure, water was added to the residue, and the mixture was extracted with ethyl acetate. The organic layer was washed with saturated brine, and dried over anhydrous magnesium sulfate. The residue was subjected to silica gel column chromatography (ethyl acetate-methanol 95:5, v/v), and crystallized from hexane-ethanol to give the title compound (41 mg, yield 25%) as colorless crystals.

¹H-NMR (300 MHz, CDCl₃) δ:0.67 (dd, J=14.0, 7.0 Hz, 3H), 0.88 (d, J=7.0 Hz, 3H), 1.64-1.80 (m, 1H), 2.32 (s, 3H), 2.58-2.85 (m, 4H), 3.06-3.30 (m, 1H), 3.48-3.53 (m, 4H), 6.70 (d, J=2.9 Hz, 1H), 6.93 (dd, J=8.6, 4.8 Hz, 1H), 7.11 (d, J=2.9 Hz, 1H), 7.20-7.24 (m, 1H), 7.66 (s, 1H).

### Example 277

### N-{2-[5-(5-chloro-1H-indol-1-yl)-1,3-dimethyl-1H-pyrazol-4-yl]ethoxy}-4-methylbenzamide

By a method similar to that in Example 274, the title compound was obtained from 1-{4-[2-(aminooxy)ethyl]-1,3-dimethyl-1H-pyrazol-5-yl}-5-chloro-1H-indole obtained in Reference Example 242 and 4-methylbenzoyl chloride.

¹H-NMR (300 MHz, CDCl₃) δ: 2.31 (s, 3H), 2.39 (s, 3H), 2.64 (t, J=6.2 Hz, 2H), 3.48 (s, 3H), 3.78-3.95 (m, 2H), 6.66 (d, J=3.4 Hz, 1H), 7.02 (d, J=8.7 Hz, 1H), 7.14-7.27 (m, 6 H), 7.64-7.66 (m, 2H).

### Example 278

### potassium {(2E)-3-[1,3-dimethyl-5-(1H-pyrrolo[2,3-b]pyridin-1-yl)-1H-pyrazol-4-yl]prop-2-enoyl}(pentylsulfonyl)azanide

By a method similar to that in Example 7, the title compound was obtained from (2E)-3-[1,3-dimethyl-5-(1H-pyrrolo[2,3-b]pyridin-1-yl)-1H-pyrazol-4-yl]-N-(pentylsulfonyl)acrylamide obtained in Example 9.

¹H-NMR (300 MHz, DMSO-d₆) δ:0.77-0.84 (m, 3H), 1.16-1.25 (m, 4H), 1.40-1.48 (m, 2H), 2.33 (s, 3H), 2.82-2.90 (m, 2H), 3.43 (s, 3H), 5.59 (d, J=16.1 Hz, 1H), 6.76 (d, J=16.1 Hz, 1H), 6.84 (d, J=3.6 Hz, 1H), 7.25 (dd, J=8.0, 4.7 Hz, 1H), 7.64 (d, J=3.6 Hz, 1H), 8.14 (dd, J=8.0, 1.5 Hz, 1H), 8.24-8.28 (m, 1H).

### Example 279

### N-[(butylamino)carbonyl]-2-[5-(5-chloro-1H-pyrrolo[2,3-b]pyridin-1-yl)-3-cyclopropyl-1-methyl-1H-pyrazol-4-yl]ethanesulfonamide

To a solution of butylamine (110 mg) in N,N-dimethylformamide (8 mL) was added N,N′-carbonyldiimidazole (265 mg), and the mixture was stirred at 60° C. for 1 hr. 2-[5-(5-Chloro-1H-pyrrolo[2,3-b]pyridin-1-yl)-3-cyclopropyl-1-methyl-1H-pyrazol-4-yl]ethanesulfonamide obtained in Reference Example 246 (350 mg), 1,8-diazabicyclo[5.4.0]undec-7-ene (259 mg) and 4-dimethylaminopyridine (209 mg) were added to the reaction mixture, and the mixture was stirred at 60° C. for 16 hr. Water was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 60:40, v/v), and crystallized from hexane-ethyl acetate to give the title compound (200 mg, yield 45%) as colorless crystals. melting point 166.8-167.6° C.

¹H-NMR (300 MHz, CDCl₃) δ:0.90-0.96 (m, 7H), 1.23-1.52 (m, 4H), 1.76-1.84 (m, 1H), 2.75-3.02 (m, 2H), 3.08-3.20 (m, 2H), 3.21-3.34 (m, 1H), 3.39 (s, 3H), 3.75-3.90 (m, 1H), 5.77 (s, 1H), 6.70 (d, J=3.6 Hz, 1H), 7.19 (d, J=3.6 Hz, 1H), 8.04 (d, J=2.3 Hz, 1H), 8.26 (d, J=2.3 Hz, 1H), 8.68 (s, 1H).

### Example 280

### butyl ({2-[5-(5-chloro-1H-pyrrolo[2,3-b]pyridin-1-yl)-3-cyclopropyl-1-methyl-1H-pyrazol-4-yl]ethyl}sulfonyl)carbamate

By a method similar to that in Example 186, the title compound was obtained from 2-[5-(5-chloro-1H-pyrrolo[2,3-b]pyridin-1-yl)-3-cyclopropyl-1-methyl-1H-pyrazol-4-yl]ethanesulfonamide obtained in Reference Example 246, butanol and N,N′-carbonyldiimidazole.

¹H-NMR (300 MHz, CDCl₃) δ:0.78-1.00 (m, 7H), 1.30-1.41 (m, 2H), 1.53-1.65 (m, 2H), 1.76-1.86 (m, 1H), 2.80-3.03 (m, 2H), 3.18-3.30 (m, 1H), 3.33 (s, 3H), 3.94-4.18 (m, 3H), 6.69 (d, J=3.6 Hz, 1H), 7.16 (d, J=3.6 Hz, 1H), 8.03 (d, J=2.3 Hz, 1H), 8.27 (d, J=2.3 Hz, 1H), 10.37 (s, 1H).

### Example 281

### 5-{2-[5-(5-chloro-1H-indol-1-yl)-1,3-dimethyl-1H-pyrazol-4-yl]ethyl}-1,2,5-thiadiazolidin-3-one 1,1-dioxide

By a method similar to that in Example 276, the title compound was obtained from 5-{2-[5-(5-chloro-1H-indol-1-yl)-1,3-dimethyl-1H-pyrazol-4-yl]ethyl}-2-(4-methoxybenzyl)-1,2,5-thiadiazolidin-3-one 1,1-dioxide obtained in Reference Example 247.

¹H-NMR (300 MHz, DMSO-d₆) δ:2.22 (s, 3H), 2.30-2.47 (m, 2H), 2.90 (t, J=7.1 Hz, 2H), 3.37 (s, 3H), 3.43-3.70 (m, 2H), 6.76 (d, J=3.4 Hz, 1H), 7.03 (d, J=8.7 Hz, 1H), 7.18 (dd, J=8.7, 2.0 Hz, 1H), 7.56 (d, J=3.4 Hz, 1H), 7.74 (d, J=2.0 Hz, 1H).

### Example 282

### (2E)-3-[1-benzyl-3-methyl-5-(1H-pyrrolo[2,3-b]pyridin-1-yl)-1H-pyrazol-4-yl]-N-(pentylsulfonyl)acrylamide

By a method similar to that in Example 1, the title compound was obtained from (2E)-3-[1-benzyl-3-methyl-5-(1H-pyrrolo[2,3-b]pyridin-1-yl)-1H-pyrazol-4-yl]acrylic acid obtained in Reference Example 251 and pentane-1-sulfonamide.

¹H-NMR (300 MHz, CDCl₃) δ:0.84-0.90 (m, 3H), 1.23-1.40 (m, 4H), 1.70-1.82 (m, 2H), 2.39 (s, 3H), 3.35-3.40 (m, 2H), 4.91 (d, J=15.6 Hz, 1H), 5.16 (d, J=15.6 Hz, 1H), 5.51 (d, J=15.9 Hz, 1H), 6.65 (d, J=3.8 Hz, 1H), 6.81-6.91 (m, 3H), 7.12-7.25 (m, 4H), 7.38 (d, J=15.9 Hz, 1H), 8.02 (dd, J=7.9, 1.5 Hz, 1H), 8.06-8.17 (m, 1H), 8.32 (dd, J=4.7, 1.5 Hz, 1H).

### Example 283

### (2E)-N-(butylsulfonyl)-3-[5-(5-chloro-1H-pyrrolo[2,3-b]pyridin-1-yl)-1,3-dimethyl-1H-pyrazol-4-yl]acrylamide

A mixture of (2E)-3-[5-(5-chloro-1H-pyrrolo[2,3-b]pyridin-1-yl)-1,3-dimethyl-1H-pyrazol-4-yl]acrylic acid obtained in Reference Example 235 (414 mg), 2-methyl-6-nitrobenzoic anhydride (547 mg), butane-1-sulfonamide (188 mg), triethylamine (412 mg), 4-dimethylaminopyridine (165 mg) and acetonitrile (12 mL) was stirred at room temperature for 20 hr. The reaction mixture was concentrated under reduced pressure, a saturated aqueous ammonium chloride solution (10 mL) was added to the residue, and the mixture was extracted with ethyl acetate. The organic layer was washed with saturated brine, dried over anhydrous magnesium sulfate, and filtrated. The filtrate was concentrated, and the residue was subjected to silica gel chromatography (hexane-ethyl acetate 30:70, v/v), and crystallized from hexane-ethanol to give the title compound (505 mg, yield 88%) as colorless crystals. melting point 245.3-248.1° C.

¹H-NMR (300 MHz, CDCl₃) δ:0.92 (t, J=7.4 Hz, 3H), 1.35-1.50 (m, 2H), 1.70-1.81 (m, 2H), 2.39 (s, 3H), 3.37-3.43 (m, 2H), 3.53 (s, 3H), 5.53 (d, J=15.7 Hz, 1H), 6.75 (d, J=3.4 Hz, 1H), 7.20 (d, J=3.4 Hz, 1H), 7.38 (d, J=15.7 Hz, 1H), 8.02 (d, J=2.3 Hz, 1H), 8.24 (d, J=2.3 Hz, 1H), 8.35 (s, 1H).

### Example 284

### (2E)-3-[5-(5-chloro-1H-pyrrolo[2,3-b]pyridin-1-yl)-1,3-dimethyl-1H-pyrazol-4-yl]-N-(propylsulfonyl)acrylamide

By a method similar to that in Example 1, the title compound was obtained from (2E)-3-[5-(5-chloro-1H-pyrrolo[2,3-b]pyridin-1-yl)-1,3-dimethyl-1H-pyrazol-4-yl]acrylic acid obtained in Reference Example 235 and propane-1-sulfonamide.

¹H-NMR (300 MHz, CDCl₃) δ:1.04 (t, J=7.4 Hz, 3H), 1.75-1.90 (m, 2H), 2.40 (s, 3H), 3.35-3.41 (m, 2H), 3.54 (s, 3H), 5.53 (d, J=15.8 Hz, 1H), 6.75 (d, J=3.6 Hz, 1H), 7.21 (d, J=3.6 Hz, 1H), 7.39 (d, J=15.8 Hz, 1H), 8.02 (d, J=2.3 Hz, 1H), 8.14 (s, 1H), 8.26 (d, J=2.3 Hz, 1H).

### Example 285

### potassium {(2E)-3-[5-(5-chloro-1H-pyrrolo[2,3-b]pyridin-1-yl)-1,3-dimethyl-1H-pyrazol-4-yl]prop-2-enoyl}(pentylsulfonyl)azanide

By a method similar to that in Example 7, the title compound was obtained from (2E)-3-[5-(5-chloro-1H-pyrrolo[2,3-b]pyridin-1-yl)-1,3-dimethyl-1H-pyrazol-4-yl]-N-(pentylsulfonyl)acrylamide obtained in Example 265.

¹H-NMR (300 MHz, DMSO-d₆) δ:0.78-0.83 (m, 3H), 1.17-1.24 (m, 4H), 1.38-1.50 (m, 2H), 2.32 (s, 3H), 2.80-2.89 (m, 2H), 3.43 (s, 3H), 5.56 (d, J=16.3 Hz, 1H), 6.75 (d, J=16.3 Hz, 1H), 6.84 (d, J=3.8 Hz, 1H), 7.77 (d, J=3.8 Hz, 1H), 8.26-8.29 (m, 2H).

### Example 286

### (2E)-3-[5-(5-bromo-1H-pyrrolo[2,3-b]pyridin-1-yl)-1,3-dimethyl-1H-pyrazol-4-yl]-N-(pentylsulfonyl)acrylamide

By a method similar to that in Example 1, the title compound was obtained from (2E)-3-[5-(5-bromo-1H-pyrrolo[2,3-b]pyridin-1-yl)-1,3-dimethyl-1H-pyrazol-4-yl]acrylic acid obtained in Reference Example 238 and pentane-1-sulfonamide.

¹H-NMR (300 MHz, CDCl₃) δ:0.88 (t, J=7.0 Hz, 3H), 1.30-1.45 (m, 4H), 1.72-1.85 (m, 2H), 2.41 (s, 3H), 3.34-3.45 (m, 2H), 3.54 (s, 3H), 5.52 (d, J=15.6 Hz, 1H), 6.75 (d, J=3.6 Hz, 1H), 7.16 (d, J=3.6 Hz, 1H), 7.39 (d, J=15.6 Hz, 1H), 8.15 (br s, 1H), 8.18 (d, J=2.1 Hz, 1H), 8.34 (d, J=2.1 Hz, 1H).

### Example 287

### (2E)-3-[5-(5-bromo-1H-pyrrolo[2,3-b]pyridin-1-yl)-1,3-dimethyl-1H-pyrazol-4-yl]-N-(butylsulfonyl)acrylamide

By a method similar to that in Example 1, the title compound was obtained from (2E)-3-[5-(5-bromo-1H-pyrrolo[2,3-b]pyridin-1-yl)-1,3-dimethyl-1H-pyrazol-4-yl]acrylic acid obtained in Reference Example 238 and butane-1-sulfonamide.

¹H-NMR (300 MHz, CDCl₃) δ:0.92 (t, J=7.3 Hz, 3H), 1.36-1.49 (m, 2H), 1.70-1.82 (m, 2H), 2.41 (s, 3H), 3.35-3.43 (m, 2H), 3.54 (s, 3H), 5.52 (d, J=15.8 Hz, 1H), 6.75 (d, J=3.8 Hz, 1H), 7.18 (d, J=3.8 Hz, 1H), 7.39 (d, J=15.8 Hz, 1H), 8.03 (br s, 1H), 8.18 (d, J=2.1 Hz, 1H), 8.34 (d, J=2.1 Hz, 1H).

### Example 288

### (2E)-N-[(butylamino)sulfonyl]-3-[5-(5-cyclopropyl-1H-pyrrolo[2,3-b]pyridin-1-yl)-1,3-dimethyl-1H-pyrazol-4-yl]acrylamide

To a mixture of (2E)-3-[5-(5-bromo-1H-pyrrolo[2,3-b]pyridin-1-yl)-1,3-dimethyl-1H-pyrazol-4-yl]-N-[(butylamino)sulfonyl]acrylamide obtained in Example 269 (395 mg), cyclopropylboronic acid (408 mg), a 2.0M aqueous sodium carbonate solution (1.6 mL) and 1,2-dimethoxyethane (10 mL) was added tetrakis(triphenylphosphine)palladium(0) (92 mg), and the reaction mixture was heated under reflux under nitrogen atmosphere for 40 hr. After the reaction mixture was allowed to cool to room temperature, water was added, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 45:55, v/v), and crystallized from hexane-ethyl acetate to give the title compound (125 mg, yield 34%) as colorless crystals.

¹H-NMR (300 MHz, CDCl₃) δ:0.74-0.80 (m, 2H), 0.89 (t, J=7.3 Hz, 3H), 1.05-1.10 (m, 2H), 1.24-1.41 (m, 2H), 1.44-1.55 (m, 2H), 1.99-2.10 (m, 1H), 2.32 (s, 3H), 2.98-3.05 (m, 2H), 3.53 (s, 3H), 5.09-5.25 (m, 1H), 5.44 (d, J=15.8 Hz, 1H), 6.67 (d, J=3.6 Hz, 1H), 7.10 (d, J=3.6 Hz, 1H), 7.34 (d, J=15.8 Hz, 1H), 7.66 (d, J=2.1 Hz, 1H), 8.15 (d, J=2.1 Hz, 1H), 8.73 (br s, 1H).

### Example 289

### (2E)-3-[5-(5-cyclopropyl-1H-pyrrolo[2,3-b]pyridin-1-yl)-1,3-dimethyl-1H-pyrazol-4-yl]-N-(pentylsulfonyl)acrylamide

By a method similar to that in Example 288, the title compound was obtained from (2E)-3-[5-(5-bromo-1H-pyrrolo[2,3-b]pyridin-1-yl)-1,3-dimethyl-1H-pyrazol-4-yl]-N-(pentylsulfonyl)acrylamide obtained in Example 286 and cyclopropylboronic acid.

¹H-NMR (300 MHz, CDCl₃) δ:0.74-0.80 (m, 2H), 0.88 (t, J=7.1 Hz, 3H), 1.07 (dd, J=8.3, 1.3 Hz, 2H), 1.26-1.47 (m, 4H), 1.72-1.86 (m, 2H), 2.00-2.12 (m, 1H), 2.30 (s, 3H), 3.39-3.45 (m, 2H), 3.52 (s, 3H), 5.51 (d, J=15.8 Hz, 1H), 6.67 (d, J=3.8 Hz, 1H), 7.10 (d, J=3.8 Hz, 1H), 7.35 (d, J=15.8 Hz, 1H), 7.66 (d, J=1.9 Hz, 1H), 8.16 (d, J=1.9 Hz, 1H), 8.81 (s, 1H).

### Example 290

### tert-butyl 5-methyl-4-{(1E)-3-oxo-3-[(pentylsulfonyl)amino]prop-1-en-1-yl}-3-(1H-pyrrolo[2,3-b]pyridin-1-yl)-1H-pyrazole-1-carboxylate

By a method similar to that in Example 1, the title compound was obtained from (2E)-3-[1-(tert-butoxycarbonyl)-5-methyl-3-(1H-pyrrolo[2,3-b]pyridin-1-yl)-1H-pyrazol-4-yl]acrylic acid obtained in Reference Example 255 and pentane-1-sulfonamide.

¹H-NMR (300 MHz, CDCl₃) δ:0.88 (t, J=7.0 Hz, 3H), 1.24-1.42 (m, 4H), 1.65 (s, 9H), 1.66-1.76 (m, 2H), 2.72 (s, 3H), 3.28-3.33 (m, 2H), 5.18 (d, J=15.7 Hz, 1H), 6.69 (d, J=3.8 Hz, 1H), 7.16 (dd, J=8.0, 4.7 Hz, 1H), 7.31-7.37 (m, 1H), 7.39 (d, J=3.8 Hz, 1H), 7.64 (d, J=15.7 Hz, 1H), 7.99 (dd, J=8.0, 1.5 Hz, 1H), 8.31 (dd, J=4.7, 1.5 Hz, 1H).

### Example 291

### (2E)-3-[5-methyl-3-(1H-pyrrolo[2,3-b]pyridin-1-yl)-1H-pyrazol-4-yl]-N-(pentylsulfonyl)acrylamide

To tert-butyl 5-methyl-4-{(1E)-3-oxo-3-[(pentylsulfonyl)amino]prop-1-en-1-yl}-3-(1H-pyrrolo[2,3-b]pyridin-1-yl)-1H-pyrazole-1-carboxylate obtained in Example 290 (321 mg) was added trifluoroacetic acid (6 mL), and the mixture was stirred at 0° C. for 90 min. The reaction mixture was concentrated under reduced pressure, an aqueous sodium hydrogencarbonate solution was added to the residue, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 15:85, v/v), and crystallized from hexane-ethanol to give the title compound (146 mg, yield 57%) as colorless crystals.

¹H-NMR (300 MHz, CDCl₃) δ:0.88 (t, J=7.0 Hz, 3H), 1.25-1.45 (m, 4H), 1.71-1.89 (m, 2H), 2.12 (s, 3H), 3.35-3.42 (m, 2H), 5.51 (d, J=15.8 Hz, 1H), 6.59 (d, J=3.6 Hz, 1H), 7.10-7.41 (m, 3H), 7.98 (d, J=7.8 Hz, 1H), 8.23 (d, J=3.6 Hz, 1H), 10.23 (s, 1H), 11.99 (s, 1H).

### Example 292

### trans-2-[5-(5-chloro-1H-indol-1-yl)-1,3-dimethyl-1H-pyrazol-4-yl]-N-(pentylsulfonyl)cyclopropanecarboxamide

To a solution of trans-2-[5-(5-chloro-1H-indol-1-yl)-1,3-dimethyl-1H-pyrazol-4-yl]cyclopropanecarboxylic acid obtained in Reference Example 148 (312 mg) in acetonitrile (5 mL) were added 1-pentanesulfonamide (144 mg), 4-dimethylaminopyridine (231 mg) and N-ethyl-N′-(3-dimethylaminopropyl)carbodiimide hydrochloride (363 mg) with stirring at room temperature, and the mixture was stirred at room temperature for 15 hr. 1N Hydrochloric acid was added to the reaction mixture, and the mixture was extracted with ethyl acetate. The organic layer was washed with 1N hydrochloric acid and saturated brine, dried over anhydrous magnesium sulfate, and filtrated. The filtrate was concentrated, and the residue was subjected to silica gel column chromatography (hexane-ethyl acetate 90:10-80:20, v/v) to give the title compound (210 mg, yield 48%) as a colorless amorphous solid.

¹H-NMR (300 MHz, CDCl₃) δ:0.70-0.84 (m, 2H), 0.86-0.95 (m, 3H), 1.14-1.52 (m, 4H), 1.60-1.84 (m, 3H), 1.95-2.21 (m, 1H), 2.31 (s, 1.5H), 2.32 (s, 1.5H), 2.86-3.01 (m, 0.5H), 3.10-3.24 (m, 0.5H), 3.26-3.37 (m, 1H), 3.54 (s, 1.5H), 3.58 (s, 1.5H), 6.74-6.84 (m, 1H), 6.86-7.05 (m, 1H), 7.14 (t, J=3.5 Hz, 1H), 7.21-7.26 (m, 1H), 7.75 (d, J=1.9 Hz, 1H).

### Example 293

### trans-N-[(butylamino)sulfonyl]-2-[5-(5-chloro-1H-indol-1-yl)-1,3-dimethyl-1H-pyrazol-4-yl]cyclopropanecarboxamide

By a method similar to that in Example 62, the title compound was obtained from trans-2-[5-(5-chloro-1H-indol-1-yl)-1,3-dimethyl-1H-pyrazol-4-yl]cyclopropanecarboxylic acid obtained in Reference Example 148 and N-butylsulfamide obtained in Reference Example 111.

¹H-NMR (300 MHz, DMSO-d₆) δ:0.31-0.60 (m, 1H), 0.73-0.90 (m, 3H), 0.83-1.00 (m, 1H), 1.14-1.49 (m, 3H), 1.60-1.75 (m, 1H), 1.81-2.04 (m, 2H), 2.19-2.25 (m, 3H), 2.57-2.93 (m, 2H), 3.34-3.43 (m, 3H), 6.76 (d, J=3.4 Hz, 1H), 6.94-7.12 (m, 1H), 7.13-7.27 (m, 1H), 7.44-7.57 (m, 1H), 7.58 (dd, J=3.2, 1.7 Hz, 1H), 7.74 (d, J=1.9 Hz, 1H), 11.24-11.66 (m, 1H).

### Example 294

### N-[(butylamino)carbonyl]-2-{1,3-dimethyl-5-[5-(trifluoromethyl)-1H-pyrrolo[2,3-b]pyridin-1-yl]-1H-pyrazol-4-yl}ethanesulfonamide

To a solution of butylamine (90.6 mg) in N,N-dimethylformamide (10 mL) was added N,N′-carbonyldiimidazole (218 mg), and the mixture was stirred at 60° C. for 1 hr. 2-{1,3-Dimethyl-5-[5-(trifluoromethyl)-1H-pyrrolo[2,3-b]pyridin-1-yl]-1H-pyrazol-4-yl}ethanesulfonamide obtained in Reference Example 208 (400 mg), 1,8-diazabicyclo[5.4.0]undec-7-ene (220 mg) and 4-dimethylaminopyridine (176 mg) were added to the reaction mixture, and the mixture was stirred at 60° C. for 18 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v), and crystallized from hexane-ethyl acetate to give the title compound (323 g, yield 64%) as colorless crystals. melting point 162.7-164.0° C.

¹H-NMR (300 MHz, CDCl₃) δ:0.89-0.96 (m, 3H), 1.26-1.40 (m, 2H), 1.41-1.52 (m, 2H), 2.32 (s, 3H), 2.78-2.92 (m, 2H), 3.07-3.24 (m, 3H), 3.43 (s, 3H), 3.64-3.80 (m, 1H), 5.59 (br s, 1H), 6.87 (d, J=3.6 Hz, 1H), 7.31 (d, J=3.6 Hz, 1H), 8.34 (d, J=1.5 Hz, 1H), 8.60 (d, J=1.3 Hz, 1H).

### Example 295

### butyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate

To a solution of butanol (91.8 mg) in N,N-dimethylformamide (10 mL) was added N,N′-carbonyldiimidazole (217 mg), and the mixture was stirred at 60° C. for 1 hr. 2-{1,3-Dimethyl-5-[5-(trifluoromethyl)-1H-pyrrolo[2,3-b]pyridin-1-yl]-1H-pyrazol-4-yl}ethanesulfonamide obtained in Reference Example 208 (400 mg), 1,8-diazabicyclo[5.4.0]undec-7-ene (220 mg) and 4-dimethylaminopyridine (176 mg) were added to this reaction mixture, and the mixture was stirred at 60° C. for 18 hr. A saturated aqueous ammonium chloride solution (10 mL) was added to the reaction mixture, and the mixture was extracted with ethyl acetate. The organic layer was washed with saturated brine, dried over anhydrous magnesium sulfate, and filtrated. The filtrate was concentrated, and the residue was subjected to silica gel column chromatography (hexane-ethyl acetate 40:60, v/v), and crystallized from hexane-ethyl acetate to give the title compound (214 mg, yield 43%) as colorless crystals. melting point 122.3-123.2° C.

¹H-NMR (300 MHz, CDCl₃) δ:0.94 (t, J=7.3 Hz, 3H), 1.30-1.44 (m, 2H), 1.54-1.66 (m, 2H), 2.32 (s, 3H), 2.74-2.90 (m, 2H), 3.11-3.23 (m, 1H), 3.37 (s, 3H), 3.91-4.18 (m, 3H), 6.85 (d, J=3.6 Hz, 1H), 7.25-7.27 (m, 1H), 8.34 (d, J=1.5 Hz, 1H), 8.61 (d, J=1.3 Hz, 1H), 10.41 (br s, 1H).

### Example 296

### ethyl ({2-[5-(5-chloro-1H-indol-1-yl)-1,3-dimethyl-1H-pyrazol-4-yl]ethyl}sulfonyl)carbamate

To a solution of 2-[5-(5-chloro-1H-indol-1-yl)-1,3-dimethyl-1H-pyrazol-4-yl]ethanesulfonamide obtained in Reference Example 179 (520 mg) in pyridine (20 mL) was added ethyl chlorocarbonate (10 mL), and the mixture was heated under reflux for 2 hr. 1N Hydrochloric acid (20 mL) was added to the reaction mixture, and the mixture was extracted with ethyl acetate. The organic layer was washed with saturated brine, dried over anhydrous magnesium sulfate, and filtrated. The filtrate was concentrated, and the residue was crystallized from hexane-ethyl acetate to give the title compound (426 mg, yield 68%) as colorless crystals.

¹H-NMR (300 MHz, CDCl₃) δ:1.23 (t, J=7.2 Hz, 3H), 2.31 (s, 3H), 2.65-2.87 (m, 2H), 3.30 (t, J=8.0 Hz, 2H), 3.47 (s, 3H), 4.11 (q, J=7.2 Hz, 2H), 6.70 (d, J=3.0 Hz, 1H), 6.92 (d, J=8.7 Hz, 1H), 7.12 (d, J=3.4 Hz, 1H), 7.20 (dd, J=8.7, 1.9 Hz, 1H), 7.67 (d, J=1.9 Hz, 1H).

### Example 297

### butyl ({2-[1,3-dimethyl-5-(5-methyl-1H-pyrrolo[2,3-b]pyridin-1-yl)-1H-pyrazol-4-yl]ethyl}sulfonyl)carbamate

By a method similar to that in Example 186, the title compound was obtained from 2-[1,3-dimethyl-5-(5-methyl-1H-pyrrolo[2,3-b]pyridin-1-yl)-1H-pyrazol-4-yl]ethanesulfonamide obtained in Reference Example 210, butanol and N,N′-carbonyldiimidazole.

¹H-NMR (300 MHz, CDCl₃) δ:0.92 (t, J=7.3 Hz, 3H), 1.25-1.42 (m, 2H), 1.52-1.64 (m, 2H), 2.31 (s, 3H), 2.47 (s, 3H), 2.81-2.87 (m, 2H), 3.07-3.18 (m, 1H), 3.31 (s, 3H), 3.85-3.96 (m, 1H), 4.00-4.17 (m, 2H), 6.65 (d, J=3.8 Hz, 1H), 7.05 (d, J=3.6 Hz, 1H), 7.85 (d, J=1.1 Hz, 1H), 8.14 (d, J=1.9 Hz, 1H), 11.91 (br s, 1H).

### Example 298

### N-[(butylamino)carbonyl]-2-[1,3-dimethyl-5-(5-methyl-1H-pyrrolo[2,3-b]pyridin-1-yl)-1H-pyrazol-4-yl]ethanesulfonamide

By a method similar to that in Example 208, the title compound was obtained from 2-[1,3-dimethyl-5-(5-methyl-1H-pyrrolo[2,3-b]pyridin-1-yl)-1H-pyrazol-4-yl]ethanesulfonamide obtained in Reference Example 210, butylamine and N,N′-carbonyldiimidazole.

¹H-NMR (300 MHz, CDCl₃) δ:0.89-0.96 (m, 3H), 1.25-1.37 (m, 2H), 1.38-1.51 (m, 2H), 2.32 (s, 3H), 2.48 (s, 3H), 2.80-2.89 (m, 2H), 3.00 (br s, 1H), 3.10-3.26 (m, 2H), 3.35 (s, 3H), 3.95 (br s, 1H), 6.67 (d, J=3.6 Hz, 1H), 7.10 (d, J=3.6 Hz, 1H), 7.89 (d, J=1.1 Hz, 1H), 8.13 (d, J=1.9 Hz, 1H).

### Example 299

### (2E)-3-{1,3-dimethyl-5-[5-(trifluoromethyl)-1H-pyrrolo[2,3-b]pyridin-1-yl]-1H-pyrazol-4-yl}-N-(propylsulfonyl)acrylamide

By a method similar to that in Example 1, the title compound was obtained from (2E)-3-{1,3-dimethyl-5-[5-(trifluoromethyl)-1H-pyrrolo[2,3-b]pyridin-1-yl]-1H-pyrazol-4-yl}acrylic acid obtained in Reference Example 203 and propane-1-sulfonamide.

¹H-NMR (300 MHz, CDCl₃) δ:1.02 (t, J=7.6 Hz, 3H), 1.72-1.86 (m, 2H), 2.42 (s, 3H), 3.33-3.39 (m, 2H), 3.57 (s, 3H), 5.65 (d, J=15.5 Hz, 1H), 6.90 (d, J=3.8 Hz, 1H), 7.32 (d, J=3.8 Hz, 1H), 7.37 (d, J=15.9 Hz, 1H), 8.32 (d, J=1.5 Hz, 1H), 8.60 (d, J=1.9 Hz, 1H).

### Example 300

### (2E)-3-{1,3-dimethyl-5-[5-(trifluoromethyl)-1H-pyrrolo[2,3-b]pyridin-1-yl]-1H-pyrazol-4-yl}-N-(pentylsulfonyl)acrylamide

By a method similar to that in Example 1, the title compound was obtained from (2E)-3-{1,3-dimethyl-5-[5-(trifluoromethyl)-1H-pyrrolo[2,3-b]pyridin-1-yl]-1H-pyrazol-4-yl}acrylic acid obtained in Reference Example 203 and pentane-1-sulfonamide.

¹H-NMR (300 MHz, CDCl₃) δ:0.83-0.91 (m, 3H), 1.24-1.43 (m, 4H), 1.69-1.82 (m, 2H), 2.42 (s, 3H), 3.33-3.42 (m, 2H), 3.57 (s, 3H), 5.63 (d, J=15.8 Hz, 1H), 6.90 (d, J=3.8 Hz, 1H), 7.32 (d, J=3.8 Hz, 1H), 7.37 (d, J=15.8 Hz, 1H), 8.32 (d, J=1.5 Hz, 1H), 8.60 (d, J=1.5 Hz, 1H).

### Example 301

### (2E)-N-[(butylamino)sulfonyl]-3-{1,3-dimethyl-5-[5-(trifluoromethyl)-1H-pyrrolo[2,3-b]pyridin-1-yl]-1H-pyrazol-4-yl}acrylamide

By a method similar to that in Example 62, the title compound was obtained from (2E)-3-{1,3-dimethyl-5-[5-(trifluoromethyl)-1H-pyrrolo[2,3-b]pyridin-1-yl]-1H-pyrazol-4-yl}acrylic acid obtained in Reference Example 203 and N-butylsulfamide obtained in Reference Example 111.

¹H-NMR (300 MHz, CDCl₃) δ:0.83-0.90 (m, 3H), 1.25-1.38 (m, 2H), 1.42-1.54 (m, 2H), 2.43 (s, 3H), 2.95 (q, J=6.8 Hz, 2H), 3.58 (s, 3H), 5.17 (t, J=5.9 Hz, 1H), 5.52 (d, J=15.9 Hz, 1H), 6.90 (d, J=3.8 Hz, 1H), 7.31-7.39 (m, 2H), 8.32 (d, J=1.9 Hz, 1H), 8.60 (d, J=1.5 Hz, 1H).

### Example 302

### (2E)-3-{1,3-dimethyl-5-[5-(trifluoromethyl)-1H-pyrrolo[2,3-b]pyridin-1-yl]-1H-pyrazol-4-yl}-N-(piperidin-1-ylsulfonyl)acrylamide

By a method similar to that in Example 1, the title compound was obtained from (2E)-3-{1,3-dimethyl-5-[5-(trifluoromethyl)-1H-pyrrolo[2,3-b]pyridin-1-yl]-1H-pyrazol-4-yl}acrylic acid obtained in Reference Example 203 and piperidine-1-sulfonamide obtained in Reference Example 177.

¹H-NMR (300 MHz, CDCl₃) δ:1.46-1.65 (m, 6H), 2.45 (s, 3H), 3.22-3.29 (m, 4H), 3.59 (s, 3H), 5.65 (d, J=15.9 Hz, 1H), 6.90 (d, J=3.4 Hz, 1H), 7.31-7.40 (m, 2H), 7.91 (br s, 1H), 8.32 (d, J=1.9 Hz, 1H), 8.61 (d, J=1.9 Hz, 1H).

### Example 303

### 3-{1,3-dimethyl-5-[5-(trifluoromethyl)-1H-pyrrolo[2,3-b]pyridin-1-yl]-1H-pyrazol-4-yl}propyl [(butylamino)sulfonyl]carbamate

By a method similar to that in Example 71, the title compound was obtained from 3-{1,3-dimethyl-5-[5-(trifluoromethyl)-1H-pyrrolo[2,3-b]pyridin-1-yl]-1H-pyrazol-4-yl}propan-1-ol obtained in Reference Example 212, chlorosulfonyl isocyanate and butylamine.

¹H-NMR (300 MHz, CDCl₃) δ:0.94 (t, J=7.3 Hz, 3H), 1.33-1.47 (m, 2H), 1.51-1.62 (m, 3H), 1.78-1.91 (m, 1H), 2.29 (s, 3H), 2.35-2.57 (m, 2H), 3.11 (q, J=6.7 Hz, 2H), 3.53 (s, 3H), 3.67-3.78 (m, 1H), 4.16-4.26 (m, 1H), 5.13 (t, J=6.0 Hz, 1H), 6.86 (d, J=3.6 Hz, 1H), 7.35 (d, J=3.6 Hz, 1H), 8.33 (d, J=1.5 Hz, 1H), 8.64 (d, J=1.3 Hz, 1H), 9.27 (s, 1H).

### Example 304

### (2E)-3-[1-benzyl-2-butyl-4-(1-naphthyl)-1H-imidazol-5-yl]-N-(pentylsulfonyl)acrylamide

By a method similar to that in Example 1, the title compound was obtained from (2E)-3-[1-benzyl-2-butyl-4-(1-naphthyl)-1H-imidazol-5-yl]acrylic acid obtained in Reference Example 258 and pentane-1-sulfonamide.

¹H-NMR (300 MHz, CDCl₃) δ:0.80-0.94 (m, 6H), 1.23-1.30 (m, 4H), 1.33-1.48 (m, 2H), 1.55-1.65 (m, 2H), 1.68-1.83 (m, 2H), 2.73-2.80 (m, 2H), 3.11-3.18 (m, 2H), 5.33 (s, 2H), 5.39 (d, J=15.5 Hz, 1H), 7.09 (d, J=7.2 Hz, 2H), 7.30-7.57 (m, 9H), 7.78 (d, J=8.0 Hz, 1H), 7.87-7.96 (m, 2H).

### Example 305

### 3-[1-benzyl-2-butyl-4-(1-naphthyl)-1H-imidazol-5-yl]-N-(pentylsulfonyl)propanamide

By a method similar to that in Example 2, the title compound was obtained from (2E)-3-[1-benzyl-2-butyl-4-(1-naphthyl)-1H-imidazol-5-yl]-N-(pentylsulfonyl)acrylamide obtained in Example 304.

¹H-NMR (300 MHz, CDCl₃) δ:0.84-0.89 (m, 6H), 1.24-1.42 (m, 6H), 1.55-1.75 (m, 4H), 1.95 (t, J=7.6 Hz, 2H), 2.63-2.76 (m, 4H), 3.04-3.12 (m, 2H), 5.18 (s, 2H), 7.04 (d, J=7.2 Hz, 2H), 7.28-7.53 (m, 7H), 7.82-7.89 (m, 3H).

### Example 306

### 3-[5-(5-chloro-1H-indol-1-yl)-1,3-dimethyl-1H-pyrazol-4-yl]propyl ethylcarbamate

To a solution of 3-[5-(5-chloro-1H-indol-1-yl)-1,3-dimethyl-1H-pyrazol-4-yl]propan-1-ol obtained in Reference Example 66 (374 mg) in pyridine (4 mL) was added ethyl isocyanate (525 mg), and the mixture was stirred at room temperature for 14 hr, and then stirred with heating at 50° C. for 5 hr. The reaction mixture was concentrated under reduced pressure, and the residue was subjected to silica gel column chromatography (hexane-ethyl acetate 30:70, v/v) to give the title compound (398 mg, yield 86%) as a colorless oil.

¹H-NMR (300 MHz, CDCl₃) δ: 1.08 (t, J=7.0 Hz, 3H), 1.53-1.63 (m, 2H), 2.19-2.38 (m, 5H), 2.90-3.20 (m, 2H), 3.45 (s, 3H), 3.70-3.92 (m, 2H), 4.28 (s, 1H), 6.67 (d, J=3.4 Hz, 1H), 6.94 (d, J=8.9 Hz, 1H), 7.10 (d, J=3.4 Hz, 1H), 7.18 (dd, J=8.9, 2.0 Hz, 1H), 7.65 (d, J=2.0 Hz, 1H).

### Example 307

### (2E)-3-(2,5-dimethyl-4-(1-naphthyl)-1-{[2-(trimethylsilyl)ethoxy]methyl}-1H-pyrrol-3-yl)-N-(pentylsulfonyl)acrylamide

By a method similar to that in Example 1, the title compound was obtained from (2E)-3-(2,5-dimethyl-4-(1-naphthyl)-1-{[2-(trimethylsilyl)ethoxy]methyl}-1H-pyrrol-3-yl)acrylic acid obtained in Reference Example 277 and pentane-1-sulfonamide.

¹H-NMR (300 MHz, CDCl₃) δ:0.00 (s, 9H), 0.79-0.87 (m, 3H), 0.90-0.97 (m, 2H), 1.20-1.29 (m, 4H), 1.54-1.65 (m, 2H), 1.96 (s, 3H), 2.45-2.48 (m, 3H), 3.11-3.20 (m, 2H), 3.53-3.60 (m, 2H), 4.83 (d, J=15.3 Hz, 1H), 5.18-5.28 (m, 2 H), 7.29-7.38 (m, 2H), 7.42-7.57 (m, 3H), 7.70-7.76 (m, 1H), 7.84-7.90 (m, 2H).

### Example 308

### cyclopropylmethyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate

By a method similar to that in Example 186, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, cyclopropylmethanol and N,N′-carbonyldiimidazole.

¹H-NMR (300 MHz, CDCl₃) δ:0.31 (t, J=5.8 Hz, 2H), 0.55-0.61 (m, 2H), 1.03-1.16 (m, 1H), 2.31 (s, 3H), 2.75-2.88 (m, 2H), 3.12-3.21 (m, 1H), 3.39 (s, 3H), 3.74-3.82 (m, 1H), 3.91-4.08 (m, 2H), 6.85 (d, J=3.6 Hz, 1H), 7.25-7.27 (m, 1H), 8.34 (d, J=1.5 Hz, 1H), 8.64 (d, J=1.5 Hz, 1H), 10.46 (br s, 1H).

### Example 309

### N-{[(cyclopropylmethyl)amino]carbonyl}-2-{1,3-dimethyl-5-[5-(trifluoromethyl)-1H-pyrrolo[2,3-b]pyridin-1-yl]-1H-pyrazol-4-yl}ethanesulfonamide

By a method similar to that in Example 208, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, cyclopropylmethylamine and N,N′-carbonyldiimidazole.

¹H-NMR (300 MHz, CDCl₃) δ:0.16-0.23 (m, 2H), 0.47-0.54 (m, 2H), 0.86-1.00 (m, 1H), 2.32 (s, 3H), 2.76-2.89 (m, 2H), 2.99 (br s, 2H), 3.19 (br s, 1H), 3.43 (s, 3H), 3.68-3.87 (m, 1H), 5.61 (br s, 1H), 6.87 (d, J=3.6 Hz, 1H), 7.31 (d, J=3.8 Hz, 1H), 8.35 (d, J=1.5 Hz, 1H), 8.61 (d, J=1.5 Hz, 1H).

### Example 310

### 3-{1,3-dimethyl-5-[5-(trifluoromethyl)-1H-pyrrolo[2,3-b]pyridin-1-yl]-1H-pyrazol-4-yl}propyl {[(2-isopropoxyethyl)amino]sulfonyl}carbamate

By a method similar to that in Example 71, the title compound was obtained from 3-{1,3-dimethyl-5-[5-(trifluoromethyl)-1H-pyrrolo[2,3-b]pyridin-1-yl]-1H-pyrazol-4-yl}propan-1-ol obtained in Reference Example 212, chlorosulfonyl isocyanate and 2-aminoethylisopropyl ether.

¹H-NMR (300 MHz, CDCl₃) δ:1.15 (d, J=6.0 Hz, 6H), 1.56 (dd, J=12.6, 6.4 Hz, 1H), 1.74-1.88 (m, 1H), 2.30 (s, 3H), 2.35-2.55 (m, 2H), 3.21-3.30 (m, 2H), 3.50-3.64 (m, 6H), 3.71-3.81 (m, 1H), 4.13-4.25 (m, 1H), 5.52 (t, J=5.9 Hz, 1H), 6.86 (d, J=3.8 Hz, 1H), 7.36 (d, J=3.6 Hz, 1H), 8.32 (d, J=1.5 Hz, 1H), 8.63 (d, J=1.3 Hz, 1H), 9.19 (s, 1H).

### Example 311

### butyl ({2-[5-(5-cyclopropyl-1H-pyrrolo[2,3-b]pyridin-1-yl)-1,3-dimethyl-1H-pyrazol-4-yl]ethyl}sulfonyl)carbamate

By a method similar to that in Example 186, the title compound was obtained from 2-[5-(5-cyclopropyl-1H-pyrrolo[2,3-b]pyridin-1-yl)-1,3-dimethyl-1H-pyrazol-4-yl]ethanesulfonamide obtained in Reference Example 282, butanol and N,N′-carbonyldiimidazole.

¹H-NMR (300 MHz, CDCl₃) δ:0.73-0.80 (m, 2H), 0.92 (t, J=7.3 Hz, 3H), 1.00-1.08 (m, 2H), 1.28-1.42 (m, 2H), 1.52-1.64 (m, 2H), 1.99-2.09 (m, 1H), 2.31 (s, 3H), 2.81-2.87 (m, 2H), 3.09-3.18 (m, 1H), 3.31 (s, 3H), 3.86-3.96 (m, 1H), 4.00-4.17 (m, 2H), 6.64 (d, J=3.6 Hz, 1H), 7.05 (d, J=3.6 Hz, 1H), 7.69 (d, J=1.9 Hz, 1H), 8.16 (d, J=2.1 Hz, 1H), 11.93 (br s, 1H).

### Example 312

### N-[(butylamino)carbonyl]-2-[5-(5-cyclopropyl-1H-pyrrolo[2,3-b]pyridin-1-yl)-1,3-dimethyl-1H-pyrazol-4-yl]ethanesulfonamide

By a method similar to that in Example 208, the title compound was obtained from 2-[5-(5-cyclopropyl-1H-pyrrolo[2,3-b]pyridin-1-yl)-1,3-dimethyl-1H-pyrazol-4-yl]ethanesulfonamide obtained in Reference Example 282, butylamine and N,N′-carbonyldiimidazole.

¹H-NMR (300 MHz, CDCl₃) δ:0.73-0.81 (m, 2H), 0.89-0.96 (m, 3H), 1.02-1.09 (m, 2H), 1.24-1.52 (m, 4H), 1.99-2.11 (m, 1H), 2.32 (s, 3H), 2.80-2.88 (m, 2H), 3.00 (br s, 1H), 3.09-3.24 (m, 2H), 3.35 (s, 3H), 3.94 (br s, 1H), 5.43 (br s, 1H), 6.66 (d, J=3.6 Hz, 1H), 7.09 (d, J=3.8 Hz, 1H), 7.72 (d, J=2.1 Hz, 1H), 8.15 (d, J=1.9 Hz, 1H).

### Example 313

### 3-methylbutyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate

By a method similar to that in Example 186, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, 3-methylbutan-1-ol and N,N′-carbonyldiimidazole.

¹H-NMR (300 MHz, CDCl₃) δ:0.93 (d, J=6.4 Hz, 6H), 1.46-1.55 (m, 2H), 1.62-1.73 (m, 1H), 2.32 (s, 3H), 2.78-2.91 (m, 2H), 3.11-3.21 (m, 1H), 3.37 (s, 3H), 3.93-4.08 (m, 2H), 4.10-4.22 (m, 1H), 6.86 (d, J=3.6 Hz, 1H), 7.25-7.28 (m, 1H), 8.34 (d, J=1.5 Hz, 1H), 8.60 (d, J=1.5 Hz, 1H), 10.46 (br s, 1H).

### Example 314

### 2-{1,3-dimethyl-5-[5-(trifluoromethyl)-1H-pyrrolo[2,3-b]pyridin-1-yl]-1H-pyrazol-4-yl}-N-{[(3-methylbutyl)amino]carbonyl}ethanesulfonamide

By a method similar to that in Example 208, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, 3-methylbutan-1-amine and N,N′-carbonyldiimidazole.

¹H-NMR (300 MHz, CDCl₃) δ:0.91 (d, J=6.6 Hz, 6H), 1.32-1.41 (m, 2H), 1.51-1.66 (m, 1H), 2.32 (s, 3H), 2.75-2.88 (m, 2H), 3.09-3.25 (m, 3H), 3.44 (s, 3H), 3.64-3.80 (m, 1H), 5.58 (br s, 1H), 6.87 (d, J=3.6 Hz, 1H), 7.31 (d, J=3.8 Hz, 1H), 8.34 (d, J=1.5 Hz, 1H), 8.59 (d, J=1.3 Hz, 1H).

### Example 315

### 2-cyclopropylethyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate

By a method similar to that in Example 186, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, 2-cyclopropylethanol and N,N′-carbonyldiimidazole.

¹H-NMR (300 MHz, CDCl₃) δ:0.05-0.11 (m, 2H), 0.43-0.50 (m, 2H), 0.62-0.76 (m, 1H), 1.46-1.55 (m, 2H), 2.32 (s, 3H), 2.77-2.90 (m, 2H), 3.12-3.23 (m, 1H), 3.37 (s, 3H), 3.96-4.09 (m, 2H), 4.14-4.26 (m, 1H), 6.86 (d, J=3.6 Hz, 1H), 7.25-7.28 (m, 1H), 8.34 (d, J=1.5 Hz, 1H), 8.60 (d, J=1.3 Hz, 1H), 10.50 (br s, 1H).

### Example 316

### (2E)-3-[5-(5-fluoro-1H-pyrrolo[2,3-b]pyridin-1-yl)-1,3-dimethyl-1H-pyrazol-4-yl]-N-(pentylsulfonyl)acrylamide

By a method similar to that in Example 1, the title compound was obtained from (2E)-3-[5-(5-fluoro-1H-pyrrolo[2,3-b]pyridin-1-yl)-1,3-dimethyl-1H-pyrazol-4-yl]acrylic acid obtained in Reference Example 285 and pentane-1-sulfonamide.

¹H-NMR (300 MHz, DMSO-d₆) δ:0.78-0.85 (m, 3H), 1.22-1.36 (m, 4H), 1.52-1.66 (m, 2H), 2.39 (s, 3H), 3.28-3.35 (m, 2H), 3.50 (s, 3H), 6.06 (d, J=16.0 Hz, 1H), 6.90 (d, J=3.6 Hz, 1H), 7.05 (d, J=16.0 Hz, 1H), 7.83 (d, J=3.8 Hz, 1H), 8.10 (dd, J=9.2, 2.8 Hz, 1H), 8.26-8.30 (m, 1H), 11.65 (br S, 1H).

### Example 317

### potassium 1-[(3-{1,3-dimethyl-5-[5-(trifluoromethyl)-1H-pyrrolo[2,3-b]pyridin-1-yl]-1H-pyrazol-4-yl}propoxy)carbonyl]-3-(2-isopropoxyethyl)diazathian-1-ide 2,2-dioxide

To a solution of 3-{1,3-dimethyl-5-[5-(trifluoromethyl)-1H-pyrrolo[2,3-b]pyridin-1-yl]-1H-pyrazol-4-yl}propyl {[(2-isopropoxyethyl)amino]sulfonyl}carbamate obtained in Example 310 (352 mg) in methanol (10 mL) was added an aqueous solution (2 mL) of potassium hydrogencarbonate (64 mg), and the mixture was stirred at room temperature for 2 hr. The reaction mixture was concentrated under reduced pressure, and gave the title compound (343 mg, yield 91%) as a colorless amorphous solid.

¹H-NMR (300 MHz, DMSO-d₆) δ:1.03 (d, J=6.0 Hz, 6H), 1.33-1.45 (m, 2H), 2.07-2.30 (m, 5H), 2.71-2.79 (m, 2H), 3.35-3.51 (m, 5H), 3.53-3.60 (m, 2H), 5.22 (t, J=6.6 Hz, 1H), 6.95 (d, J=3.6 Hz, 1H), 7.88 (d, J=3.6 Hz, 1H), 8.57 (d, J=1.5 Hz, 1H), 8.62 (s, 1H).

### Example 318

### isobutyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate

By a method similar to that in Example 186, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, 2-methylpropan-1-ol and N,N′-carbonyldiimidazole.

¹H-NMR (300 MHz, CDCl₃) δ:0.93 (d, J=6.8 Hz, 6H), 1.83-1.98 (m, 1H), 2.32 (s, 3H), 2.78-2.90 (m, 2H), 3.11-3.22 (m, 1H), 3.36 (s, 3H), 3.69-3.79 (m, 1H), 3.89-4.14 (m, 2H), 6.86 (d, J=3.8 Hz, 1H), 7.27 (s, 1H), 8.34 (d, J=1.5 Hz, 1H), 8.60 (d, J=1.5 Hz, 1H), 10.35 (s, 1H).

### Example 319

### 2-{1,3-dimethyl-5-[5-(trifluoromethyl)-1H-pyrrolo[2,3-b]pyridin-1-yl]-1H-pyrazol-4-yl}-N-[(isobutylamino)carbonyl]ethanesulfonamide

By a method similar to that in Example 208, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, isobutylamine and N,N′-carbonyldiimidazole.

¹H-NMR (300 MHz, CDCl₃) δ:0.89 (d, J=6.6 Hz, 6H), 1.67-1.81 (m, 1H), 2.32 (s, 3H), 2.76-3.05 (m, 4H), 3.20 (br s, 1H), 3.43 (s, 3H), 3.67 (br s, 1H), 5.74 (br s, 1H), 6.87 (d, J=3.8 Hz, 1H), 7.31 (d, J=3.8 Hz, 1H), 8.34 (d, J=1.5 Hz, 1H), 8.59 (d, J=1.3 Hz, 1H).

### Example 320

### propyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate

By a method similar to that in Example 186, the title compound was obtained from 2-{1,3-dimethyl-5-[5-(trifluoromethyl)-1H-pyrrolo[2,3-b]pyridin-1-yl]-1H-pyrazol-4-yl}ethanesulfonamide obtained in Reference Example 208, propanol and N,N′-carbonyldiimidazole.

¹H-NMR (300 MHz, CDCl₃) δ:0.94 (t, J=7.4 Hz, 3H), 1.60-1.71 (m, 2H), 2.32 (s, 3H), 2.77-2.89 (m, 2H), 3.11-3.21 (m, 1H), 3.37 (s, 3H), 3.89-4.13 (m, 3H), 6.86 (d, J=3.6 Hz, 1H), 7.24-7.27 (m, 1H), 8.34 (d, J=1.7 Hz, 1H), 8.61 (d, J=1.5 Hz, 1H), 10.46 (s, 1H).

### Example 321

### 3-[1,3-dimethyl-5-(5-methyl-1H-pyrrolo[2,3-b]pyridin-1-yl)-1H-pyrazol-4-yl]propyl {[(2-isopropoxyethyl)amino]sulfonyl}carbamate

By a method similar to that in Example 71, the title compound was obtained from 3-[1,3-dimethyl-5-(5-methyl-1H-pyrrolo[2,3-b]pyridin-1-yl)-1H-pyrazol-4-yl]propan-1-ol obtained in Reference Example 289, chlorosulfonyl isocyanate and 2-aminoethylisopropyl ether.

¹H-NMR (300 MHz, CDCl₃) δ:1.16 (d, J=2.4 Hz, 3H), 1.18 (d, J=2.4 Hz, 3H), 1.40-1.54 (m, 1H), 1.83-1.96 (m, 1H), 2.28 (s, 3H), 2.41-2.64 (m, 5H), 3.23-3.31 (m, 2H), 3.51 (s, 3H), 3.55-3.66 (m, 4H), 4.25-4.35 (m, 1H), 5.54 (t, J=5.8 Hz, 1H), 6.65 (d, J=3.6 Hz, 1H), 7.16 (d, J=3.6 Hz, 1H), 7.84-7.86 (m, 1H), 8.22 (d, J=1.9 Hz, 1H), 10.85 (br s, 1H).

### Experimental Example 1

### PPARγ-RXRα Heterodimer Ligand Activity

The PPARγ:RXRα:4ERPP/CHO-K1 cells described in WO03/099793 were cultured in a Ham F12 medium [manufactured by Life Technologies, Inc., US] containing 10% calf fetal serum [manufactured by Life Technologies, Inc., US], sown in a 96-well white plate [manufactured by Corning Coster Corporation, US] at 1×10⁴ cells/well, and incubated overnight in a carbon dioxide gas incubator at 37° C.

Then, the medium was removed from the 96 well white plate, 45 μl of Ham F12 medium containing 0.1% fatty acid-free bovine serum albumin (BSA) and a test compound (5 μl) were added, and the cells were incubated for one day in a carbon dioxide gas incubator at 37° C. The medium was removed, 20 μl of PicaGene 7.5 (manufactured by Wako Pure Chemical Industries, Ltd.) 2-fold diluted with HBSS (HANKS' BALANCED SALT SOLUTION) [manufactured by BIO WHITTAKER, US] was added. After stirring, the luciferase activity was determined using the 1420 ARVO Multilabel Counter [manufactured by PerkinElmer, US].

The induction rate was calculated from the luciferase activity of each test compound based on the luciferase activity of the test compound non-administration group as 1. The test compound concentration and the induction rate were analyzed by PRISM [manufactured by GraphPad Software, Inc., US] to calculate EC₅₀ value (compound concentration showing 50% of the maximum value of induction rate) of the test compound. The results are shown in Table 1.

TABLE 1

| Test compound | EC₅₀ |
|---|---|
| (Example No.) | (nM) |
| 6 | 19 |
| 7 | 39 |
| 9 | 64 |
| 24 | 54 |
| 27 | 6.1 |
| 31 | 5.5 |
| 33 | 88 |
| 40 | 12 |
| 55 | 44 |
| 62 | 37 |
| 66 | 22 |
| 82 | 67 |
| 84 | 220 |
| 99 | 24 |
| 189 | 54 |
| 197 | 86 |
| 232 | 15 |
| 264 | 26 |
| 279 | 60 |
| 283 | 14 |
| 294 | 32 |
| 295 | 22 |

As shown above, the compound of the present invention has been shown to have a superior PPARγ-RXRα heterodimer ligand activity.

### Formulation Example 1

### Production of Capsule

| | |
|---|---|
| 1) compound of Example 1 | 30 mg |
| 2) finely divided powder cellulose | 10 mg |
| 3) lactose | 19 mg |
| 4) magnesium stearate | 1 mg |
| total | 60 mg |

1), 2), 3) and 4) are mixed and filled in a gelatin capsule.

### Formulation Example 2

### Production of Tablet

| | |
|---|---|
| 1) compound of Example 1 | 30 g |
| 2) lactose | 50 g |
| 3) cornstarch | 15 g |
| 4) calcium carboxymethylcellulose | 44 g |
| 5) magnesium stearate | 1 g |
| 1000 tablets total | 140 g |

The total amount of 1), 2), 3) and 30 g of 4) are kneaded with water, vacuum dried and sized. The sized powder is mixed with 14 g of 4) and 1 g of 5) and the mixture is punched out with a tableting machine. In this way, 1000 tablets containing 30 mg of the compound of Example 1 per tablet are obtained.

### INDUSTRIAL APPLICABILITY

The compound of the present invention is useful for an agent for the prophylaxis or treatment of diabetes, which has a superior hypoglycemic action, and is associated with a fewer side effects such as body weight gain and the like.

This application is based on patent application No. 31221/2007 filed in Japan, the contents of which are hereby incorporated by reference.

1. A compound represented by the formula (I′): 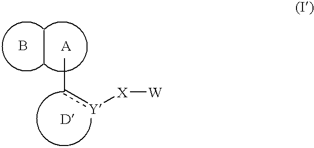
wherein
ring A and ring B are the same or different and each is an optionally substituted 5- to 7-membered monocycle;
ring D′ is an optionally substituted 5-membered monocyclic aromatic heterocycle wherein Y′ is N or C;
X is a spacer having 1 to 4 atoms in the main chain; and
W is a group represented by
—CONR^{1a}S(O)ₘR²,
—CONR^{1a}S(O)ₘOR²,
—CONR^{1a}CONR^{1c}R²,
—CONR^{1a}S(O)ₘNR^{1c}R²,
—NR^{1b}CONR^{1a}S(O)ₘR²,
—NR^{1b}S(O)ₘNR^{1a}COₙR²,
—S(O)ₘNR^{1a}COₙR²,
—S(O)ₘNR^{1a}CONR^{1c}R²,
—OCONR^{1a}S(O)ₘR²,
—OCONR^{1a}S(O)ₘNR^{1c}R²,
—ONR^{1a}COₙR²,
—OCONR^{1c}R², or
—ONR^{1a}CONR^{1c}R²
wherein
R^{1a} and R^{1b} are the same or different and each is a hydrogen atom or a C₁₋₆ alkyl group;
R^{1c} is a hydrogen atom, a C₁₋₆ alkyl group or a C₁₋₆ alkoxy group;
R² is a hydrogen atom, an optionally substituted hydrocarbon group or an optionally substituted heterocyclic group; and
m and n are the same or different and each is an integer of 1 or 2, or
a 5- or 6-membered heterocyclic group containing NH, which is optionally substituted, provided that
1) when ring D′ is a substituted imidazole, then W should not be 2-amino-1H-imidazol-5-yl, 1H-imidazol-2-yl, 3,5-dimethyl-1H-pyrazol-4-yl and piperazin-1-yl;
2) when ring D′ is a substituted pyrazole, and X is —CH═, then W should not be 4-oxo-2-thioxo-1,3-thiazolidin-5-ylidene, 5-oxo-2-thioxoimidazolidin-4-ylidene optionally substituted by phenyl group(s), 3-methyl-5-oxo-1,5-dihydro-4H-pyrazol-4-ylidene, 2,4,6-trioxotetrahydropyrimidin-5(2H)-ylidene and 4,6-dioxo-2-thioxotetrahydropyrimidin-5(2H)-ylidene; and
3) 5-(6-methoxy-2-naphthyl)-1-(pyrrolidin-2-ylmethyl)-1H-1,2,3-triazole is excluded, or a salt thereof.

2. A compound represented by the formula (I): 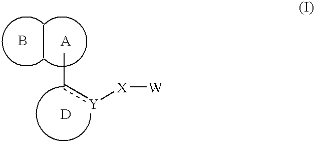
wherein
ring A and ring B are the same or different and each is an optionally substituted 5- to 7-membered monocycle;
ring D is an optionally substituted 5-membered monocycle wherein Y is N, C or CH;
X is a spacer having 1 to 4 atoms in the main chain; and
W is a group represented by
—CONR^{1a}S(O)ₘR²,
—CONR^{1a}CONR^{1c}R²,
—CONR^{1a}S(O)ₘNR^{1c}R²,
—NR^{1b}CONR^{1a}S(O)ₘR²,
—S(O)ₘNR^{1a}COₙR²,
—OCONR^{1a}S(O)ₘR²,
—OCONR^{1a}S(O)ₘNR^{1c}R²,
—ONR^{1a}COₙR²,
—OCONR^{1c}R², or
—ONR^{1a}CONR^{1c}R²
wherein
R^{1a} and R^{1b} are the same or different and each is a hydrogen atom or a C₁₋₆ alkyl group;
R^{1c} is a hydrogen atom, a C₁₋₆ alkyl group or a C₁₋₆ alkoxy group;
R² is a hydrogen atom, an optionally substituted hydrocarbon group or an optionally substituted heterocyclic group; and
m and n are the same or different and each is an integer of 1 or 2, or
a 5- or 6-membered heterocyclic group containing NH, which is optionally substituted, provided that
1) when ring D is a substituted imidazole, then W should not be an aminoimidazole; and
2) when ring D is a substituted pyrazole, and X is —CH═, then W should not be an oxothioxothiazolidinyl and an oxothioxoimidazolidinyl, or a salt thereof.

3. The compound of claim 1, wherein ring D′ is an optionally substituted pyrazole.

4. The compound of claim 2, wherein ring D is an optionally substituted pyrazole.

5. The compound of claim 1, wherein X is a C₁₋₄ alkylene group or a C₂₋₄ alkenylene group.

6. The compound of claim 1, wherein W is a group represented by —CONR^{1a}S(O)ₘR² wherein each symbol is as defined in claim 1.

7. (2E)-3-[1,3-dimethyl-5-(1H-pyrrolo[2,3-b]pyridin-1-yl)-1H-pyrazol-4-yl]-N-(pentylsulfonyl)acrylamide (Example 9),
(2E)-3-[5-(5-chloro-1H-indol-1-yl)-1,3-dimethyl-1H-pyrazol-4-yl]-N-(pentylsulfonyl)acrylamide (Example 27),
(2E)-3-[1,3-dimethyl-5-(1H-pyrrolo[2,3-b]pyridin-1-yl)-1H-pyrazol-4-yl]-N-[(4-methylphenyl)sulfonyl]acrylamide (Example 33),
(2E)-3-[5-(5-chloro-1H-indol-1-yl)-1,3-dimethyl-1H-pyrazol-4-yl]-N-[(pentylamino)sulfonyl]acrylamide (Example 62),
cyclopropylmethyl ({2-[5-(5-chloro-1H-indol-1-yl)-1,3-dimethyl-1H-pyrazol-4-yl]ethyl}sulfonyl)carbamate (Example 189),
butyl ({2-[5-(5-chloro-1H-pyrrolo[2,3-b]pyridin-1-yl)-1-methyl-3-(trifluoromethyl)-1H-pyrazol-4-yl]ethyl}sulfonyl)carbamate (Example 197),
(2E)-3-[1,3-dimethyl-5-(5-methyl-1H-pyrrolo[2,3-b]pyridin-1-yl)-1H-pyrazol-4-yl]-N-(pentylsulfonyl)acrylamide (Example 232),
(2E)-3-[5-(5-chloro-1H-pyrrolo[2,3-b]pyridin-1-yl)-1,3-dimethyl-1H-pyrazol-4-yl]-N-{[(cyclopropylmethyl)amino]sulfonyl}acrylamide (Example 264),
N-[(butylamino)carbonyl]-2-[5-(5-chloro-1H-pyrrolo[2,3-b]pyridin-1-yl)-3-cyclopropyl-1-methyl-1H-pyrazol-4-yl]ethanesulfonamide (Example 279),
(2E)-N-(butylsulfonyl)-3-[5-(5-chloro-1H-pyrrolo[2,3-b]pyridin-1-yl)-1,3-dimethyl-1H-pyrazol-4-yl]acrylamide (Example 283),
N-[(butylamino)carbonyl]-2-{1,3-dimethyl-5-[5-(trifluoromethyl)-1H-pyrrolo[2,3-b]pyridin-1-yl]-1H-pyrazol-4-yl}ethanesulfonamide (Example 294), or
butyl [(2-{1,3-dimethyl-5-[5-(trifluoromethyl)-1H-pyrrolo[2,3-b]pyridin-1-yl]-1H-pyrazol-4-yl}ethyl)sulfonyl]carbamate (Example 295),
or a salt thereof.

8. A prodrug of a compound of claim 1.

9. A pharmaceutical agent comprising a compound of claim 1 or a prodrug thereof.

10. The pharmaceutical agent of claim 9, which is an insulin sensitizer.

11. The pharmaceutical agent of claim 9, which is an agent for the prophylaxis or treatment of diabetes.

12. A method of improving insulin resistance in a mammal, which comprises administering a compound of claim 1 or a prodrug thereof to the mammal.

13. A method for the prophylaxis or treatment of diabetes in a mammal, which comprises administering a compound of claim 1 or a prodrug thereof to the mammal.

14-15. (canceled)

16. The compound of claim 2, wherein X is a C₁₋₄ alkylene group or a C₂₋₄ alkenylene group.

17. The compound of claim 2, wherein W is a group represented by —CONR^{1a}S(O)ₘR² wherein each symbol is as defined in claim 2.
